(19) United States
(12) Patent Application Publication
(10) Pub. No.: US 2011/0008347 A1
(43) Pub. Date: Jan. 13, 2011

(54) CANCER-RELATED PROTEIN KINASES

(75) Inventors: Axel Ullrich, Munich (DE); Jens Ruhe, Planegg (DE); Stefan Hart, Singapore (SG); Sylvia Street, Planegg (DE); Chee Hong Wong, Singapore (SG); Boon Tin Chua, Singapore (SG); Kiat Han Ho, Singapore (SG)

Correspondence Address: FOLEY & LARDNER LLP, P.O. BOX 80278, SAN DIEGO, CA, 92138-0278, US

(73) Assignee: Agency for Science ,Technology and Research

(21) Appl. No.: 12/517,050

(22) PCT Filed: Dec. 3, 2007
(86) PCT No.: PCT/SG2007/000412

§ 371 Date: Sep. 23, 2010

Related U.S. Application Data
(60) Provisional application No. 60/868,173, filed on Dec. 1, 2006.

(51) Int. Cl. A61K 39/395; C07H 21/00; C12Q 1/68; G01N 33/53; C07H 21/04; C12N 5/10; C12N 9/12; G01N 33/573; A61P 43/00

(52) U.S. Cl. 424/139.1; 536/23.2; 435/6; 435/7.21; 536/24.3; 435/325; 435/194; 435/7.4

(57) ABSTRACT

The present invention relates to mutant kinase polypeptides and kinase variants selected from the group consisting of AATYK (AATK), ABL1, ACK1, ALK, ARG, AXL, BMX, BRK, BTK, CCK4, CSFR1, CSK, DDR1, DDR2, EGFR, EPHA1, EPHA2, EPHA3, EPHA4, EPHA5, EPHA6, EPHA7, EPHA10, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MATK, MER, MET, NTRK1, NTRK2, NTRK3, PDGRFA, PDGFRB, PTK-9, PYK2, RET, RON, ROR1, ROR2, ROS, RYK, STYK, SYK, TEC, TEK, TIE, TNK1, TXK, TYK2, TYRO3, VEGFR1, VEGFR2, VEGFR3, YES1, and ZAP70, nucleotide sequences encoding the mutant kinase polypeptides and kinase variants, as well as various products and methods useful for the diagnosis and treatment of various kinase-related diseases and conditions, including the screening for and identification of novel protein kinase modulators.

### CROSS-REFERENCE TO RELATED APPLICATIONS

This application makes reference to and claims the benefit of priority of an application for “Cancer-Related Protein Kinases” filed on Dec. 1, 2006 with the United States Patent and Trademark Office and there duly assigned the U.S. Ser. No. 60/868,173. The contents of said application filed on Dec. 1, 2006 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to mutant protein kinases, nucleotide sequences encoding the mutated protein kinases, their use for the diagnosis and treatment of various kinase-related diseases and conditions and the design and identification of novel protein kinase inhibitors.

### BACKGROUND OF THE INVENTION

The following description of the background of the invention is provided to aid in understanding the invention, but is not admitted to be or to describe prior art to the invention.

Cancer is the second most common cause of death in developed countries and is a rising health problem in less developed parts of the world. The diagnosis of cancer is connected to great physical and mental suffering for affected individuals and poses a significant burden on the health care system. For many tumors, conventional management strategies, such as surgery, radiation therapy and chemotherapy, have high toxicity with limited efficacy. Thus, an in-depth understanding of the molecular genetics underlying individual malignancies will greatly facilitate the cancer therapeutic problem is now commonly accepted among investigators in the field.

Autonomous cell growth resulting in tissue invasion and metastasis is the defining feature of all malignant neoplasms. Cancers do not necessarily arise solely as a result of an accelerated rate of cell proliferation. Rather they are the consequence of an imbalance between the rate of cell-cycle progression (cell division) and cell growth (cell mass) on one hand and programmed cell death (apoptosis) on the other. Researchers now recognize that aberrant cellular signal transduction pathways play a vital role in driving this imbalance and hence in malignant transformation.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function.

Thus, one of the most critical groups of signaling molecules involved in normal and abnormal cellular regulation are the protein kinases, a family of enzymes that catalyze the phosphorylation of amino acid residues of various target molecules. This process controls fundamental cellular processes including cell cycle, migration, metabolism, proliferation, differentiation, and survival.

Protein kinases are one of the largest families of eukaryotic proteins with several hundred known members. These proteins share a 250-300 amino acid long kinase domain that can be subdivided into 12 distinct subdomains that includes the common catalytic core structure. These conserved protein motifs have recently been exploited using PCR-based cloning strategies leading to a significant expansion of the known kinases.

The best-characterized protein kinases in eukaryotes phosphorylate proteins on the hydroxyl moiety of serine, threonine and/or tyrosine residues. These kinases largely fall into two groups, those specific for phosphorylating serines and threonines, and those specific for phosphorylating tyrosines. Some kinases, referred to as “dual specificity” kinases, are able to phosphorylate on tyrosine as well as serine/threonine residues.

Protein kinases can also be characterized by their location within the cell. Some kinases are transmembrane receptor-type proteins capable of directly altering their catalytic activity in response to the external environment such as the binding of a ligand. Others are non-receptor-type proteins lacking any transmembrane domain. They can be found in a variety of cellular compartments from the inner surface of the cell membrane to the nucleus.

Many kinases are involved in regulatory cascades wherein their substrates may include other kinases whose activities are regulated by their phosphorylation state. Ultimately the activity of some downstream effector is modulated by phosphorylation resulting from activation of such a pathway.

Protein tyrosine phosphorylation, mediated by protein tyrosine kinases, is a key mechanism underlying signal transduction pathways that regulate fundamental cellular processes such as proliferation, differentiation, motility and cell survival. Deregulation of kinase activity, caused by genetic alterations, modulated expression levels, or the loss of negative regulatory control mechanisms has been described for various members of the tyrosine kinase family, and in many cases has been implicated in the development of human cancer (Blume-Jensen, P., & Hunter, T., Nature 411, 355-365 (2001)). Consequently, tyrosine kinases have become rational targets for therapeutic intervention using both monoclonal antibodies and small molecule drugs.

First hints for genetically modified tyrosine kinases to be involved in the development of cancer came from viral oncogenes which in several cases have been shown to represent altered versions of cellular receptor tyrosine kinases. The avian erythroblastosis gene v-erbB for example has been identified as a truncated and mutated version of the human epidermal growth factor receptor EGFR (e.g. Downward, J., et al. Nature 311, 483-485, (1984)), which for the first time connected an animal oncogene with a human gene that encoded a cell growth controlling membrane protein, Furthermore, v-fms was found to represent a deleted form of the macrophage CSF-1 receptor (Coussens, L., et al. Nature 320, 277-280 (1986); Sherr, C. J., et al., Cell 41, 665-676 (1985)), and the identified truncations, deletions and mutations were speculated to form the genetic basis for the conversion of a proto-oncogene into an oncogene that can cause malignant cancer in animals.

These observations stimulated a massive search for genetic alterations of tyrosine kinases in human cancer. Several deletions and point mutations were described that result in increased catalytic activity of the EGFR. The most prevalent in tumors was found to be EGFRvIII, an EGFR deletion mutant that lacks exons 2-7, which can arise from gene rearrangement or alternative mRNA splicing (Malden, L. T., et al., Cancer Res 48, 2711-2714 (1988)). Amplification of the HER2 gene (Coussens, L., et al., Science 230, 1132-1139 (1985))—another member of the EGFR family—was discovered as a genetic abnormality occurring in 30% of invasive human breast cancer, and a significant correlation between HER2 overexpression in tumors and reduced patient survival could be demonstrated (Slamon, D. J., et al., Science 235, 177-182 (1987)). These findings established HER2 as a prognostic marker and led to evaluate the concept of target-specific cancer therapy. In 1998, it culminated in the FDA-approval of Herceptin, a humanized monoclonal against HER2 and the first targeted anti-kinase therapeutic agent based on genomic research.

Since then it became more and more obvious that even single genetic changes were able to mediate oncogenic potential to a given kinase. This was first shown for neu, the rat homologue of the human HER2 gene, where the replacement of a single valine within the transmembrane domain by glutamic acid resulted in activation of p185 and tumorigenic activity of the modified neu proto-oncogene (Bargmann, C. I., et al., Cell 45, 649-657 (1986)). Other very well known genetic variations in tyrosine kinase genes that are associated with cancer are the BCR-ABL oncogene, a reciprocal translocation between chromosomes 9 and 22 in chronic myelogenous leukaemia (CML), and KIT receptor point mutations in gastrointestinal stromal tumors (GISTs) (Corless, C. L., et al., J Mol. Diagn. 6, 366-370 (2004))—both being targeted by the small molecule imatinib (Gleevec, Novartis) (Demetri, G. D., Eur. J. Cancer 38 Suppl 5, S52-S59 (2002); Peggs, K. & Mackinnon, S., N. Engl. J. Med. 348, 1048-1050 (2003)).

In addition to their significant role in disease initiation or progression, specific mutations could be shown to mediate and thus predict sensitivity towards specific small molecule inhibitors such as imatinib or gefitinib (Iressa, AstraZeneca). Two independent groups recently described the identification of mutations clustered around the ATP binding pocket of the EGFR kinase domain and demonstrated their occurrence primarily in patients with Iressa-responsive lung cancer (Lynch, T. J., et al., N. Engl. J. Med. 350, 2129-2139 (2004); Paez, J. G., et al., Science 304, 1497-1500 (2004)).

Therefore, significant efforts have been undertaken to screen for mutations in tyrosine kinase encoding genes on the level of genomic DNA, and comprehensive studies focusing on the kinase domain in colorectal cancer (Bardelli, A., et al., Science 300, 949 (2003)) or selected tumor types (Bignell, G., et al., Genes, Chromosomes & Cancer 45:42-46 (2006) (2006); Davies, H., et al., et al., Cancer Res. 65:17, 7591-7595 (2005); Stephens, P., et al., Nature 431:525-526 (2004)) have recently been reported.

For decades the traditional approach to identify cancer genes was to hand-pick likely candidates and then search for mutations within them. But since the completion of the draft human genome sequence in 2000, scientists have had the tools to go after cancer mutations in a more global and systematic way.

Scientists of the post-genome era can examine the sequences of thousands of genes in cancer cells. But because of the expense and technical limitations of current sequencing technologies, groups have for now been focusing on specific sets of genes rather than attempting to go after all of them at once.

DNA sequencing, although a major component, is only one part of the process of finding cancer mutations. Indeed cancer can arise from small mutations in the DNA sequences, changes in the number of copies of specific genes, rearrangements affecting entire chromosomes, and even from tumor viruses that land inside or next to human genes. In addition, epigenetic changes are also thought to play a role in cancer.

However, the large variation in molecular changes from one tumor to another, and even within the same tumor from one cell to another, has long been an obstacle for the development of effective therapies. Recent studies provided the first clear demonstration of the vast array of mutations present in cancer cells and identified close to 200 mutations in protein kinase genes in lung tumors, indicating that many mutated protein kinases may be contributing to lung cancer development but that mutations in any one gene are infrequent.

For some tumors one will thus find high frequency mutations that will make good drug targets, but many will be a mixture of different low frequency mutations.

Beyond the challenge of obtaining accurate DNA sequence on such large scale, researches have to sift through the hundreds of changes identified in any one set of genes and determine which are specific to cancer and which are normal changes that occur in DNA of any individual (polymorphisms).

This problem can be addressed by comparing each cancer sequence to that of DNA taken from normal cells of the same patient. Polymorphisms should be present in both samples whereas cancer-specific changes should be present only in the cancer DNA.

The present invention relates to the identification of protein kinase mutations prevalent in human malignancies as well as methods of use of such mutated protein kinases. The invention further relates to germline variations in kinase genes that are related to tumor development and progression.

### SUMMARY OF THE INVENTION

Thus, in a first aspect the invention provides an isolated, enriched, or purified nucleic acid molecule. The nucleic acid molecule encodes a mutant of a protein kinase polypeptide. The protein kinase is one of FGFR4, FGFR1, Tyro3, TEC, CSK and Ack1. Further, the mutant of the protein kinase polypeptide encoded by the nucleic acid molecule includes at least one mutation of FGFR4 Y367C, FGFR1 P252S, Tyro3 S531L, Tyro3 P822L, TEC L89R, TEC W531R, TEC P587L, CSK Q26X and ACK1 S985N.

In a second aspect invention provides a method of identifying a cell that is resistant to apoptosis inducing reagents, i.e. a cell that is chemoresistant. The method includes measuring in the cell the expression of the protein kinase Tyro3, or identifying the amino acid at position 531 and/or 822 of the expressed protein kinase Tyro3, or identifying the amino acid at position 89, 531 and/or 587 of the expressed protein kinase TEC. Where the method includes measuring in the cell the expression of the protein kinase Tyro3 the result of the measurement obtained is further compared with that of a control measurement. An increased expression of protein kinase Tyro3 indicates resistance of the cell to apoptosis inducing reagents. Where the method includes identifying the amino acid at position 531 and/or 822 of the expressed protein kinase Tyro3, the presence of Leucine at position 531 instead of Serine and/or the presence of Leucine at position 822 instead of Proline indicates increased resistance of the cell to apoptosis inducing reagents. Where the method includes identifying the amino acid at position 89, 531 and/or 587 of the expressed protein kinase Tec, the presence of Arginine at position 89 instead of Leucine, the presence of Arginine at position 531 instead of Tryptophan, and/or the presence of Leucine at position 587 instead of Proline indicates increased resistance of the cell to apoptosis inducing reagents.

In a third aspect invention provides a method of identifying a cell that has a predisposition to transform into a cancer cell. The method includes either identifying the amino acid at position 367 of the expressed protein kinase FGFR4 and/or the amino acid at position 252 of the expressed protein kinase FGFR1, or identifying the amino acid at position 362 of the expressed protein kinase TYK2, or identifying the amino acid at position 26 of the expressed protein kinase C-terminal Src kinase (CSK), and/or identifying the amino acid at position 985 of the expressed protein kinase Ack1. Where the method includes identifying the amino acid at position 367 of the expressed protein kinase FGFR4 and/or the amino acid at position 252 of the expressed protein kinase FGFR1, the presence of Cysteine at position 367 of the expressed protein kinase FGFR4 instead of Tyrosine and/or the presence of Serine at position 252 of the expressed protein kinase FGFR1 instead of Proline indicates an increased predisposition to transform into a cancer cell. Where the method includes identifying the amino acid at position 362 of the expressed protein kinase TYK2, the presence of Phenylalanine at position 362 of the expressed protein kinase TYK2 instead of Valine indicates an increased predisposition to transform into a cancer cell. Where the method includes identifying the amino acid at position 26 of the expressed protein kinase C-terminal Src kinase (CSK), the presence of an amino acid different from Glutamine at position 26 of the expressed protein kinase CSK indicates an increased predisposition to transform into a cancer cell. Where the method includes identifying the amino acid at position 985 of the expressed protein kinase Ack1, the presence of Asparagine at position 985 of the expressed protein kinase Ack1 instead of Serine indicates an increased predisposition to transform into a cancer cell.

In a fourth aspect the invention provides an isolated, enriched, or purified nucleic acid molecule encoding a mutant kinase polypeptide selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70.

The mutant kinase polypeptides encoded by the nucleic acid molecules according to the invention include at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N2671, BRK W78fsX58, BTK M4891, BTK W588C, CCK4 D106N, CCK4 T410S, CCK4 M746L, CCK4 Q913H, CSK Q26X, DDR1R60C, DDR1 V100A, DDR1 R248W, DDR2 M117I, DDR2 R478c, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR2 I526T, FGFR4Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L9911, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V6221, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2D390fsX46, ROR2 P548S, ROS R187M, ROS D709fsX16, ROS Q865fsX90, ROS A1443S, RYK H250R, RYK R504H, RYK A559T, SYK M34fsX3, SYK 1262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V.

In some embodiments, the invention features isolated, enriched, or purified nucleic acid molecules encoding a mutant kinase polypeptide comprising, consisting essentially of or consisting of a nucleotide sequence that: (a) encodes a polypeptide having the amino acid sequence set forth in SEQ ID Nos: 1-256 or any variant, isoform or fragment thereof, with the proviso that the mutated position or region is retained; (b) is the complement of the nucleotide sequence of (a).

In another embodiment, the invention is directed to an isolated, enriched, or purified nucleic acid molecule encoding a kinase polypeptide variant selected from the group consisting of AATYK (AATK), ACK1, AXL, CCK4, EPHA1, EPHA2, EPHA3, EPHB3, FAK, FES, HER2, LMTK2 (AATYK2/BREK), MATK, MER, NTRK3, PDGRFA, PDGFRB, PTK-9, PYK2, RON, ROS, RYK, TEK, TNK1, TXK, TYK2, VEGFR1, VEGFR2, VEGFR3, and ZAP70.

The kinase variant encoded by the nucleic acid molecules according to the invention includes at least one of the germline alterations AATYK G600C, AATYK G641S, AATYK F1163S, AATYK T1227M, ACK1 P725L, AXL G517S, CCK4 P693L, CCK4 A777V, CCK4 S795R, EPHA1 S936L, EPHA2 R876H, EPHA3 I564V, EPHB3 R514Q, FAK L926delins-PWRL, FES P397R, FES S72_K129del, FES E413fsX131, HER2 R1161Q, LMTK2 S910I, MATK A496T, MER E823Q, NTRK3 E402_F410delinsV, NTRK3 G466_Y529delinsD, NTRK3 R711_V712ins16, PDGFRA L221F, PDGFRA S478P, PDGFRB T464M, PTK-9 E195_V196insRPEDHIG, PYK2 G414V, RON Q473_D515del, RON R627fsX23, RON R813_C814insQ, ROS C76fsX, RYK F516L, TEK V600L, TNK1 D472_R473del, TNK1 M598fsX5, TXK R63C, TXK Y414fsX15, TYK2 E971fsX67, VEGFR1 Y642H, VEGFR1 E982A, VEGFR1 P1201L, VEGFR2 C482R, VEGFR3 R1321Q and ZAP70 K186fsX,

In some embodiments, the invention features isolated, enriched, or purified nucleic acid molecules encoding a kinase variant comprising, consisting essentially of or consisting of a nucleotide sequence that: (a) encodes a polypeptide having the amino acid sequence set forth in SEQ ID Nos: 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641 or any variant, isoform or fragment thereof, with the proviso that the altered position or region is retained; (b) is the complement of the nucleotide sequence of (a).

The nucleic acid may be isolated from a natural source by cDNA cloning or by subtractive hybridization. The natural source may be mammalian, for example, of murine, human, porcine, canine, or bovine origin. The polypeptide can be isolated from every suitable sample, including cultured cells, a biopsy, blood, semen, or any tissue derived from an organ, for example, skin, liver, pancreas to name only a few illustrative examples. In another aspect, the nucleic acid may be synthesized by the triester method or by using an automated DNA synthesizer.

In other embodiments, the invention features isolated, enriched, or purified nucleic acid molecules encoding mutant kinase polypeptides, further comprising a vector or promoter effective to initiate transcription in a host cell.

In a fifth aspect the invention also features a recombinant nucleic acid, for instance in a cell or an organism. The recombinant nucleic acid may include, consist essentially of or consist of a sequence set forth in SEQ ID Nos:257-512, 643-646, 649, 654-655, 657-658, 663, 667-668, 673, 677-680, 692, 701-703, 713-717, 719-721, 728, 730-731, 737, 746, 750-751, 753-754, 756, 760, 762-764, 767, and 770-771, or a functional derivative thereof and, optionally, a vector or a promoter effective to initiate transcription in a host cell. The recombinant nucleic acid can alternatively contain a transcriptional initiation region functional in a cell, a sequence complementary to an RNA sequence encoding a kinase polypeptide and a transcriptional termination region functional in a cell. Specific vectors and host cell combinations are discussed herein.

In yet other embodiments, the nucleic acid is useful for the design of hybridization probes to facilitate identification and cloning of mutated kinase polypeptides or kinase variants, the design of PCR probes to facilitate cloning of mutated kinase polypeptides or kinase variants, obtaining antibodies to mutated kinase polypeptide or kinase variants, and designing antisense oligonucleotides.

In a sixth aspect, the invention provides a nucleic acid probe for the detection of a nucleic acid that encodes a mutant kinase polypeptide in a sample. The mutant kinase polypeptide is selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, comprising at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M5691, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR 1646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V6221, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S91, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V. The nucleic acid probe may include, consist essentially of or consist of a nucleotide base sequence that will hybridize to the mutated region of the nucleic acid sequence set forth in any of SEQ ID Nos: 257-512 or a functional derivative thereof.

In a seventh aspect, the present invention also features a nucleic acid probe for the detection of nucleic acid that encodes a kinase variant in a sample. The altered kinase polypeptide is one of AATYK (AATK), ACK1, AXL, CCK4, EPHA1, EPHA2, EPHA3, EPHB3, FAK, FES, HER2, LMTK2 (AATYK2/BREK), MATK, MER, NTRK3, PDGRFA, PDGFRB, PTK-9, PYK2, RON, ROS, RYK, TEK, TNK1, TXK, TYK2, VEGFR1, VEGFR2, VEGFR3, and ZAP70 comprising at least one of the alterations AATYK G600C, AATYK G641S, AATYK F1163S, AATYK T1227M, ACK1 P725L, AXL G517S, CCK4 P693L, CCK4 A777V, CCK4 S795R, EPHA1 S936L, EPHA2 R876H, EPHA3 I564V, EPHB3 R514Q, FAK L926delinsPWRL, FES P397R, FES S72_K129del, FES E413fsX131, HER2 R1161Q, LMTK2 S910I, MATK A496T, MER E823Q, NTRK3 E402_F410delinsV, NTRK3 G466_Y529delinsD, NTRK3 R711_V712ins16, PDGFRA L221F, PDGFRA S478P, PDGFRB T464M, PTK-9 E195_V196insRPEDHIG, PYK2 G414V, RON Q473_D515del, RON R627fsX23, RON R813_C814insQ, ROS C76fsX, RYK F516L, TEK V600L, TNK1 D472_R473del, TNK1 M598fsX5, TXK R63C, TXK Y414fsX15, TYK2 E971fsX67, VEGFR1 Y642H, VEGFR1 E982A, VEGFR1 P1201L, VEGFR2 C482R, VEGFR3 R1321Q and ZAP70 K186fsX. In some embodiments, the nucleic acid probe includes, consists essentially of or consists of a nucleotide base sequence that will hybridize to the mutated region of the nucleic acid sequence set forth in any of SEQ ID Nos: 643-646, 649, 654-655, 657-658, 663, 667-668, 673, 677-680, 692, 701-703, 713-717, 719-721, 728, 730-731, 737, 746, 750-751, 753-754, 756, 760, 762-764, 767, and 770-771, or a functional derivative thereof.

Methods for using the probes of the invention include detecting the presence or amount of mutated or altered kinase RNA in a sample by contacting the sample with a nucleic acid probe under conditions such that hybridization occurs and detecting the presence or amount of the probe bound to kinase RNA. The nucleic acid duplex formed between the probe and a nucleic acid sequence coding for a kinase polypeptide may be used in the identification of the sequence of the nucleic acid detected. In certain embodiment, kits for performing such methods may be constructed to include a container means having disposed therein a nucleic acid probe.

The present invention also relates to the use of a set of the mutant kinase polypeptides, the nucleic acids encoding the mutant kinase polypeptides, and the nucleic acid probes of the invention as molecular markers for the diagnosis of proliferative diseases or disorders in a subject. Such a method may also be useful to predict the risk of cancer with high predictive accuracy and/or to choose an adequate therapy. Moreover, such a method may also be useful to monitor the course of a treatment regimen and/or to predict the risk or cancer recurrence.

Thus, the present invention also encompasses a method that allows predicting or diagnosing proliferative diseases or disorders, such as cancer, in a subject comprising the steps of (a) obtaining a biological sample from the subject; and (b) detecting the expression of one or more nucleic acid molecules encoding the mutant kinase polypeptides of the invention in said sample.

In one embodiment of the invention, these two or more nucleic acid molecules the expression of which is to be detected includes, consist essentially of or consist of at least one of the nucleotide sequences set forth in SEQ ID Nos: 257-512, or complements and fragments thereof. Such a combination of two ore more of these molecular markers may be utilized for the risk prediction or diagnosis of cancer in a subject. Any combination of at least two of the above nucleic acid molecules may be used for this analysis. For example, in some embodiments, a combination of at least 2, 3, 4, 5, 6, 8, 10, 12, 14, 16, 18, 20, 25, 30, 40, 50, 60, 70, 80, 90 or more of the nucleotide sequences set forth in SEQ ID Nos: 257-512 may be used for said diagnostic purpose.

In another aspect, the invention is also directed to the use of a set of kinase variants, i.e. kinases that include germline alterations such as single nucleotide polymorphisms, the nucleic acids encoding these kinase variants, and nucleic acid probes for the nucleic acids encoding these kinase variants as molecular markers for the diagnosis of proliferative diseases or disorders in a subject. Such a method may also be useful to predict the risk of cancer development and/or metastasis with high predictive accuracy. Further, such method may also allow to choose an adequate therapy, monitor the course of a treatment regimen and/or to predict the risk or cancer recurrence.

Suitable kinase variants include, but are not limited to AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V6451, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2H199_Q247del, FGFR3 T311_Q422del, FGFR4 V10I, FGFR4 L136P, FGFR4 G388R, FLT3 M227T, FRK G122R, FYN D506E, HER2 I655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V870I, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, Styk G2045, TEK P346Q, TEK, V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del

Thus, the present invention also encompasses a method that allows predicting or diagnosing proliferative diseases or disorders, such as cancer, in a subject. The method includes the steps of (a) obtaining a biological sample from the subject; and (b) detecting the expression of one or more nucleic acid molecules encoding the kinase variants of the invention in said sample.

In one embodiment of the invention, these one or more nucleic acid molecules the expression of which is to be detected include, consist essentially of or consist of at least one of the nucleotide sequences set forth in SEQ ID Nos: 643-772 or complements and fragments thereof. Such a combination of two or more of these molecular markers may be utilized for the risk prediction or diagnosis of cancer in a subject. Any combination of at least two of the above nucleic acid molecules may be used for this analysis. For example, in some embodiments, a combination of at least 5, 7, 10, 15, 20, 30, 40, 50, 60, 70, 80, 90 or all 91 of the nucleotide sequences set forth in SEQ ID Nos: 643-772 may be used for said diagnostic purpose.

In still another aspect, the invention provides a recombinant cell or tissue comprising a nucleic acid molecule encoding a kinase polypeptide selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S91, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2P1280S, YES1 K113Q, ZAP70 T155M, and ZAP70 M549V. In such cells, the nucleic acid may be under the control of the genomic regulatory elements, or may be under the control of one or more heterologous regulatory elements including a heterologous promoter. In certain embodiments, the kinase polypeptide is a fragment of the protein encoded by the amino acid sequence set forth in SEQ ID Nos: 1-256, or the corresponding full-length amino acid sequence, wherein said fragment includes the mutated region.

Alternatively, the present invention provides a recombinant cell or tissue comprising a nucleic acid molecule encoding a kinase polypeptide selected from the group consisting of AATYK (AATK), ABL1, ACK1, ALK, ARG, AXL, CCK4, CSFR1, EGFR, EPHA1, EPHA10, EPHA2, EPHA3, EPHA7, EPHB2, EPHB3, EPHB4, EPHB6, FAK, FES, FGFR1, FGFR2, FGFR3, FGFR4, FLT3, FRK, FYN, HER2, HER3, JAK2, JAK3, LMTK2 (AATYK2/BREK), MATK, MER, MET, NTRK1, NTRK2, NTRK3, PDGRFA, PDGFRB, PTK-9, PYK2, RET, RON, ROR1, ROR2, ROS, RYK, STYK, TEK, TNK1, TXK, TYK2, TYRO3, VEGFR1, VEGFR2, VEGFR3 and ZAP70 including at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2H199_Q247del, FGFR3 T311_Q422del, FGFR4 V10I, FGFR4 L136P, FGFR4 G388R, FLT3 M227T, FRK G122R, FYN D506E, HER21655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S9101, MATK A496T, MER E823Q, MER V870I, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del. In such cells, the nucleic acid may be under the control of the genomic regulatory elements, or may be under the control of one or more heterologous regulatory elements including a heterologous promoter. In certain embodiments, the kinase polypeptide is a fragment of the protein encoded by the amino acid sequence set forth in SEQ ID Nos: 513-642, or the corresponding full-length amino acid sequence, wherein said fragment includes the mutated region.

In still another aspect, the invention provides an isolated, enriched, or purified mutant kinase polypeptide selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR 1646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901 S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V.

In some embodiments, the mutant kinase polypeptide is a fragment of the protein with the amino acid sequence set forth in SEQ ID Nos: 1-256, or the corresponding full-length amino acid sequences, with the proviso that the mutation is included in said fragment. Also included are variants and isoforms of the mutant kinases of the invention.

The invention further provides an isolated, enriched, or purified kinase variant selected from the group consisting of AATYK (AATK), ACK1, AXL, CCK4, EPHA1, EPHA2, EPHA3, EPHB3, FAK, FES, HER2, LMTK2 (AATYK2/BREK), MATK, MER, NTRK3, PDGRFA, PDGFRB, PTK-9, PYK2, RON, ROS, RYK, TEK, TNK1, TXK, TYK2, VEGFR1, VEGFR2, VEGFR3, and ZAP70 including at least one of the alterations AATYK G600C, AATYK G641S, AATYK F1163S, AATYK T1227M, ACK1 P725L, AXL G517S, CCK4 P693L, CCK4 A777V, CCK4 S795R, EPHA1 S936L, EPHA2 R876H, EPHA3 I564V, EPHB3 R514Q, FAK L926delinsPWRL, FES P397R, FES S72_K129del, FES E413fsX131, HER2 R1161Q, LMTK2 S910I, MATK A496T, MER E823Q, NTRK3 E402_F410delinsV, NTRK3 G466_Y529delinsD, NTRK3 R711_V712ins16, PDGFRA L221F, PDGFRA S478P, PDGFRB T464M, PTK-9 E195_V196insRPEDHIG, PYK2 G414V, RON Q473_D515del, RON R627fsX23, RON R813_C814insQ, ROS C76fsX, RYK F516L, TEK V600L, TNK1 D472_R473del, TNK1 M598fsX5, TXK R63C, TXK Y414fsX15, TYK2 E971fsX67, VEGFR1 Y642H, VEGFR1 E982A, VEGFR1 P1201L, VEGFR2 C482R, VEGFR3 R1321Q and ZAP70 K186fsX.

In certain embodiments, the kinase variant is a fragment of the protein with the amino acid sequence set forth in SEQ ID Nos.: 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641, or the corresponding full-length amino acid sequences as well as isoforms thereof, with the proviso that the mutation is included in said fragment.

The polypeptide can be isolated from a natural source by methods well-known in the art. The natural source may be mammalian, for example, of murine, human, porcine, canine, or bovine origin. The polypeptide can be isolated from every suitable sample, including cultured cells, a biopsy, blood, semen, or any tissue derived from an organ, for example, skin, liver, pancreas to name only a few illustrative examples. In another embodiment the polypeptide may be synthesized using an automated polypeptide synthesizer.

In certain embodiments the invention includes the above mutant kinases and kinase variants, wherein the mutant kinases or kinase variants are of recombinant origin. For example, the mutant kinases and kinase variants of the invention may be expressed in a heterologous expression system.

In a further aspect, the invention provides an antibody (e.g., a monoclonal or polyclonal antibody) having specific binding affinity only for a mutant kinase polypeptide or a mutant kinase polypeptide domain or fragment, where the polypeptide is selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR2 I526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V.

Also included in the present invention are antibodies (e.g., monoclonal or polyclonal antibodies) having specific binding affinity only for a kinase variant or a kinase variant domain or fragment, where the polypeptide is selected from the group consisting of the or the group AATYK (AATK), ABL1, ACK1, ALK, ARG, AXL, CCK4, CSFR1, EGFR, EPHA1, EPHA10, EPHA2, EPHA3, EPHA7, EPHB2, EPHB3, EPHB4, EPHB6, FAK, FES, FGFR1, FGFR2, FGFR3, FGFR4, FLT3, FRK, FYN, HER2, HER3, JAK2, JAK3, LMTK2 (AATYK2/BREK), MATK, MER, MET, NTRK1, NTRK2, NTRK3, PDGRFA, PDGFRB, PTK-9, PYK2, RET, RON, ROR1, ROR2, ROS, RYK, STYK, TEK, TNK1, TXK, TYK2, TYRO3, VEGFR1, VEGFR2, VEGFR3 and ZAP70 including at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R 14362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2H199_Q247del, FGFR3 T311_Q422del, FGFR4 V10I, FGFR4 L136P, FGFR4 G388R, FLT3 M227T, FRK G122R, FYN D506E, HER21655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V870I, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del.

Antibodies or antibody fragments having specific binding affinity only for a mutant kinase polypeptide or a kinase variant of the invention may be used in methods for detecting the presence and/or amount of mutant kinase polypeptide or kinase variant in a sample by probing the sample with the antibody under conditions suitable for kinase-antibody immuno-complex formation and detecting the presence and/or amount of the antibody conjugated to the kinase polypeptide. The antibodies of the invention are thus capable of differentiating between the mutant/variant and the native form of a kinase polypeptide.

Diagnostic kits for performing such methods may be constructed to include antibodies or antibody fragments specific for the kinase as well as a conjugate of a binding partner of the antibodies or the antibodies themselves. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

An antibody or antibody fragment with specific binding affinity only for a mutant kinase polypeptide or a kinase variant of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

In a further aspect, the invention relates to methods for identifying a compound that modulates kinase activity comprising: (a) contacting a kinase polypeptide selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V7471, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S3291, FAK Q440R, FAK A472V, FAK P901S, FER 1240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T17I, MET P366S, MET S691L, NTRK1 P453fsX15, NTRK1 L585fsX73, NTRK1 G595E, NTRK1 R748W, NTRK2 A586V, NTRK2 V6221, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V, with a test substance; (b) measuring the activity of said polypeptide; and (c) determining whether said substance modulates the activity of said polypeptide.

In addition to applying the above method to the mutant kinase polypeptides of the invention, such a method may also be suitable to test compounds for their activity for the kinase variants of the invention. These kinase variants include, but are not limited to AATYK (AATK), ABL1, ACK1, ALK, ARG, AXL, CCK4, CSFR1, EGFR, EPHA1, EPHA10, EPHA2, EPHA3, EPHA7, EPHB2, EPHB3, EPHB4, EPHB6, FAK, FES, FGFR1, FGFR2, FGFR3, FGFR4, FLT3, FRK, FYN, HER2, HER3, JAK2, JAK3, LMTK2 (AATYK2/BREK), MATK, MER, MET, NTRK1, NTRK2, NTRK3, PDGRFA, PDGFRB, PTK-9, PYK2, RET, RON, ROR1, ROR2, ROS, RYK, STYK, TEK, TNK1, TXK, TYK2, TYRO3, VEGFR1, VEGFR2, VEGFR3 and ZAP70. These kinases include at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2 H199_Q247del, FGFR3 T311_Q422del, FGFR4 V10I, FGFR4 L136P, FGFR4 G388R, FLT3 M227T, FRK G122R, FYN D506E, HER2 I655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V870I, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del.

In another aspect, the invention refers to methods for identifying a substance that modulates kinase activity in a cell comprising the steps of: (a) expressing a kinase polypeptide in a cell, wherein said polypeptide is selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S91, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V, (b) adding a test substance to said cell; and (c) monitoring a change in cell phenotype or the interaction between said polypeptide and a natural binding partner.

Alternatively, kinase variants may be used in such a method, wherein the kinase variants include at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2H199_Q247del, FGFR3 T311_Q422del, FGFR4 V10I, FGFR4 L136P, FGFR4 G388R, FLT3 M227T, FRK G122R, FYN D506E, HER21655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V870I, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del.

In yet another aspect, the invention provides methods for treating or preventing a proliferative disease or disorder by administering to a patient in need of such treatment a substance that modulates the activity of a mutant kinase selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70. The mutant kinase includes at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V. In some embodiments the disease is cancer.

In another embodiment, the method for treating or preventing a proliferative disease or disorder includes administering to a patient in need of such treatment a substance that modulates the activity of a kinase variant associated with such a disease or disorder, the kinase variant being selected from the group consisting of AATYK (AATK), ABL1, ACK1, ALK, ARG, AXL, CCK4, CSFR1, EGFR, EPHA1, EPHA10, EPHA2, EPHA3, EPHA7, EPHB2, EPHB3, EPHB4, EPHB6, FAK, FES, FGFR1, FGFR2, FGFR3, FGFR4, FLT3, FRK, FYN, HER2, HER3, JAK2, JAK3, LMTK2 (AATYK2/BREK), MATK, MER, MET, NTRK1, NTRK2, NTRK3, PDGRFA, PDGFRB, PTK-9, PYK2, RET, RON, ROR1, ROR2, ROS, RYK, STYK, TEK, TNK1, TXK, TYK2, TYRO3, VEGFR1, VEGFR2, VEGFR3 and ZAP70. These kinases include at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2M71T, FGFR2H199_Q247del, FGFR3 T311_Q422del, FGFR4 V10I, FGFR4 L136P, FGFR4 G388R, FLT3 M227T, FRK G122R, FYN D506E, HER2 I655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V8701, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V4861, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del.

The present invention also provides a method for screening for human cells containing a mutant kinase polypeptide of the invention or an equivalent sequence. The method involves identifying the mutant polypeptide in human cells using techniques that are routine and standard in the art, such as those described herein for identifying the mutant kinases of the invention (e.g., cloning, Southern or Northern blot analysis, in situ hybridization, PCR amplification, etc.).

Thus, in a further aspect, the invention encompasses methods for the detection of a nucleic acid encoding a mutant kinase polypeptide or a kinase variant in a sample as a diagnostic tool for diseases or disorders, wherein the method includes the steps of (a) contacting the sample with a nucleic acid probe which hybridizes under hybridization assay conditions to

(i) a nucleic acid target region of a mutant kinase polypeptide selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, with these mutant kinases including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M6571, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR2 I526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T17I, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S91, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V; or

(ii) a nucleic acid target region of a kinase variant selected from the group consisting of AATYK (AATK), ABL1, ACK1, ALK, ARG, AXL, CCK4, CSFR1, EGFR, EPHA1, EPHA10, EPHA2, EPHA3, EPHA7, EPHB2, EPHB3, EPHB4, EPHB6, FAK, FES, FGFR1, FGFR2, FGFR3, FGFR4, FLT3, FRK, FYN, HER2, HER3, JAK2, JAK3, LMTK2 (AATYK2/BREK), MATK, MER, MET, NTRK1, NTRK2, NTRK3, PDGRFA, PDGFRB, PTK-9, PYK2, RET, RON, ROR1, ROR2, ROS, RYK, STYK, TEK, TNK1, TXK, TYK2, TYRO3, VEGFR1, VEGFR2, VEGFR3 and ZAP70, with these kinases including at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2H199_Q247del, FGFR3 T311_Q422del, FGFR4 V10I, FGFR4 L136P, FGFR4 G388R, FLT3 M227T, FRK G122R, FYN D506E, HER21655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V870I, MET N375S, MET R988C, MET T1010I, MET V1238I, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del. The probe includes the nucleic acid sequence that encodes the mutant kinase polypeptide or the kinase variant, fragments thereof, or the complements of the sequences and fragments; and (b) detecting the presence or amount of the probe: target region hybrid as an indication of the disease.

In certain embodiments of the invention, the disease or disorder is a proliferative disease or disorder, for example, cancer.

In certain embodiments the nucleic acid probes of the invention hybridizes to a kinase target region encoding at least 6, 12, 75, 90, 105, 120, 150, 200, 250, 300 or 350 contiguous amino acids of the sequence set forth in SEQ ID Nos: 1-256 and 513-642, or the corresponding full-length amino acid sequence, or a functional derivative thereof, with the proviso that the mutated region is included. Hybridization conditions should be such that hybridization occurs only with the kinase genes in the presence of other nucleic acid molecules. Under stringent hybridization conditions only highly complementary nucleic acid sequences hybridize. Typically, such conditions prevent hybridization of nucleic acids having one or more mismatches in 20 contiguous nucleotides.

The diseases that could be diagnosed by detection of mutated or altered kinase nucleic acid in a sample may include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well-known in the art and can be readily adapted in order to obtain a sample that is compatible with the method utilized.

The summary of the invention described above is not limiting and other features and advantages of the invention will be apparent from the following detailed description of the invention, and from the claims.

### BRIEF DESCRIPTION OF THE DRAWINGS

The invention will be better understood with reference to the detailed description when considered in conjunction with the non-limiting examples and the accompanying drawings, in which:

FIG. 1A shows the origin of tissue samples and number of tumor cell lines derived thereof, FIG. 1B patterns of genetic alterations for 5 skin-derived tumor cell lines and FIG. 1C the number of somatic (bright) or germline (dark) alterations in the characterization of the tyrosine kinase transcriptome of tumor cell lines.

FIG. 2 illustrates the genetic alterations of the FGFR4 gene detected in tumor cell lines and control samples.

FIG. 3A depicts the rates of germline alterations of non-synonymous polymorphisms identified in the TKT of 276 cancer cell lines and control samples (MS=missense substitution, NS=nonsense substitution, DEL=deletion, INS=insertions). FIG. 3B shows the domain localization of identified polymorphisms. FIG. 3C depicts the tissue distribution of germline variations (BL=bladder; BS=bone and soft tissue; BA=brain; BE=breast; CV=cervix and vulva; CO=colon; EP=endometrium and placenta; HN=head and neck; HL=hematopoietic and lymphoid system; KI=kidney; LI=liver; LU=lung; OV=ovary; PA=pancreas; PR=prostate; SK=skin; ST=stomach; TE=testes; TY=thyroid, NO=normal control samples).

FIG. 4 depicts the diverging occurrence rates of polymorphisms in different tumor types and/or control samples. The frequency of homozygous (HO; dark bar) and heterozygous (HE; light bar) carriers of (A) EGFR R521K, (B) TYK2 V362F and (C) TNK1 M598delinsEVRSHX was determined. Only tissue origins (for abbreviations see legend to FIG. 1, supra) with an expression of the corresponding gene in at least 10 samples have been selected for this analysis.

FIG. 5 depicts the distributions of non-synonymous somatic mutations identified in all transcribed PTK genes from 254 tumor cell lines. A, rates of somatic mutations. The allocation to missense (MS) or nonsense (NS) substitutions, deletions (DEL) and insertions (INS) as well as frequency categories (1, 2-5, 6-10 or more than 10 affected samples) is shown. B, domain localization of identified mutations. The localization within defined domains or other protein regions is indicated. C, tissue distribution of sporadic alterations. For each somatic mutation, the tissue distribution (see legend to FIG. 3 for abbreviations) was determined (FIG. 35) and presented here for text-related examples. Paired numerals indicate the number of mutated and expression-positive cell lines within a given tumor type. Novel somatic mutations are highlighted in bold type.

FIG. 6 is an illustration of known and novel genetic alterations in selected genes. A, SYK. The domain organization and location of genetic alterations is displayed. B, sequence comparison of FGFR1-4. For FGFR1-4, the general domain organization (middle) and sequence comparisons of the linker region connecting the IG-D2- and IG-D3-domain (top) as well as a part of the extracellular juxtamembrane region (bottom) are illustrated. Genetic alterations identified in the cell line screen are illustrated below, known sequence variants are depicted above the graphical representation of the domain structure. Polymorphisms are underlined and somatic mutations are not highlighted. Numbers in parenthesis indicate the number of affected non-related cell lines. (SH2: Src Homolgy 2 Domain; TK: Tyrosine Kinase Domain; S: Signal Peptide; TM: Transmembrane Domain; IG: Immunoglobulin-Like Domain).

FIG. 7 shows overexpression of FGFR4 in hepatocellular carcinoma (HCC) patients (n=57) in the tumor vs. the adjacent normal tissue as determined by real-time PCR.

FIG. 8 shows the corresponding Ct values of the detected FGFR4 overexpression shown in FIG. 7.

FIG. 9 shows that a single nucleotide polymorphism, G388R is highly represented in the Asian population, including HCC patients.

FIG. 10 depicts alpha-fetoprotein (AFP) levels in HCC patients, an oncofetal protein serving as a diagnostic marker for hepatocellular carcinoma. In patients with hepatoma, the incidence of elevated AFP levels correlates with tumor burden (60-70% of HCC patients exhibit AFP elevation). FIG. 10 shows that the homozygous 388Arg genotype correlates with an increased AFP secretion at the point of tumor resection.

FIG. 11 shows elevated AFP production caused by stimulation of FGFR4 in the HCC cell line HuH7 using 50 and 100 ng/ml of the specific ligand FGF19.

FIG. 12 shows elevated AFP production caused by stimulation of FGFR4 in the HCC cell line HepG2 using 50 and 100 ng/ml of the specific ligand FGF19.

FIG. 13 depicts gene silencing of FGFR4 by siRNA. A=control siRNA, B=FGFR4 siRNA.

FIG. 14 shows AFP production in HuH7 cells after gene silencing of FGFR4 by siRNA.

FIG. 15 shows AFP production in HuH7 cells after exposure to 0, 1, 5 and 10 μM of the commercially available non-selective FGFR inhibitor PD173074. SF=serum free

FIG. 16 depicts the viability of HuH7 exposed to the FGFR inhibitor PD173074. An exquisite anti-proliferative effect can be observed.

FIG. 17 depicts a decreased Tyrosinphosphorylation of TEC mutants. Reduced tyrosine phosphorylation was observed for TEC L89R, TEC W531R and TEC P587L, but not TEC R563K (IP=immunoprecipitation, IB=immunoblot, α-HA=anti Hemagglutinin antibody, α-PY=anti phosphor tyrosin antibody).

FIG. 18 illustrates the genetic alterations of TEC-kinase identified by the present inventors (TEC L89R, TEC W531R and TEC P587L).

FIG. 19 shows a decreased MAPK signaling of TEC mutants indicated by decreased MAPK phosphorylation. No activation of the MAPK pathway was observed for TEC L89R, TEC W531R and TEC P587L.

FIG. 20 depicts a c-fos gene reporter assay (HEK293 cells, 18 h), performed to compare TEC wt with TEC mutants. TEC kinase is involved in B-cell receptor induced c-fos promoter activity (Aoki, N., et al., J Biol. Chem. 2004 Mar. 12; 279(11):10765-10775 (2004), Epub 2003 Dec. 16). Overexpression of TEC wt and TEC R563K showed enhanced luciferase expression but not TEC L89R, TEC W531R, TEC P589L and TEC KM.

FIG. 21 depicts a decreased Stat3 activation of the TEC mutants.

FIG. 22 shows an in vitro Ubiquitination assay Hek293 transfected with myc-Ubiquitin and Flag-protein of interest. An exchange of amino acid 985 from Serine to Asparagine resulted in a protein of higher stability that is less sensitive to ubiquitination.

FIG. 23 shows the overexpression of TYK2 mutants in HEK293 cells.

FIG. 24 depicts tumor cell lines, control cell lines, and tissues from healthy individuals. The name, origin, reference number, and supplier/source of the tumor cell lines, normal cell lines, and tissues from healthy individuals analyzed in the screen are specified. Related tumor cell lines are indicated by parenthesized asterisks and identical numerals. (ATCC: The American Type Culture Collection, Manassas (VA), USA; DKFZ: Tumorbank Deutsches Krebsforschungszentrum, Heidelberg, Germany; DSMZ: German Collection of Microorganisms and Cell Cultures, Braunschweig, Germany; ECACC: The European Collection of Cell Cultures, Porton Down, Salisbury, UK)

FIG. 25 lists the primary tumor samples used, identifiers for the 55 primary kidney, prostate and breast cancer samples are listed.

FIG. 26 shows the primers used for PCR amplification and sequencing of PTK genes.

FIG. 27 shows the primers used for PCR amplification and sequencing of PTK gene fragments using genomic DNA as template.

FIG. 28 indicates the NCBI accession numbers of PTK reference sequences. Accession numbers of NCBI (http://www.ncbi.nlm.nih.gov) sequence files that served as references for sequences alignments are listed.

FIG. 29 lists the genetic alterations identified in all cDNA samples. Sequence differences occurring in all cDNA samples analyzed are shown. References for previously described alterations are provided. Homozygocity (HO) or heterozygocity (HE) is indicated.

FIG. 30 shows the characterization of each tumor- and control cell line with regard to somatic and germline alterations in the transcripts of PTK genes. Somatic mutations are underlined.

FIG. 31 depicts the characterization of PTK genes with regard to identified somatic and germline alterations in the transcripts of 276 tumor cell lines and control samples. For each tyrosine kinase gene, the spectrum of identified genetic alterations and the corresponding patterns of affected tumor cell lines or control samples are presented. Receptor- and non-receptor tyrosine kinase genes reflect the categorization into subfamilies, cancer cell lines are subdivided according to their tissue origin. The total sample number carrying a given sequence variant is indicated. Heterozygocity is indicated by a hash.

FIG. 32 lists the SEQ ID Nos for both the amino acid sequences (“protein”) and the nucleic acid sequences (“nt”) of the identified somatic alterations.

FIG. 33 lists the SEQ ID Nos for both the amino acid sequences (“protein”) and the nucleic acid sequences (“nt”) of the identified germline alterations.

FIG. 34 specifies the frameshift alterations and insertions. Detailed amino acid sequence information is provided for selected frameshift (fs) alterations and insertions. Nomenclature of sequence alterations is based on suggestions by the Human Genome Variation Society (HGVS; Kong-Beltran, M., et al., Cancer Res, 66: 283-289 (2006)).

FIG. 35 depicts the domain localization and tissue distribution of identified polymorphisms (abbreviations for the Ig-like domain: [A]=H199_Q247delins48; [B]=T311_Q422del (Bounacer, et al, 2002, supra)*).

FIG. 36 shows the genetic alterations analyzed in primary tumor samples.

FIG. 37 depicts the domain localization and tissue distribution of identified somatic mutations.

FIG. 38 depicts the absolute numbers of somatic mutations in transcribed PTK genes from 254 tumor cell lines.

FIG. 39 depicts the normalized mutational frequencies of transcribed PTK genes. Somatic mutation frequencies—normalized with respect to the expression status among the 254 tumor cell lines—are provided for each PTK gene and expressed as number of mutations per 1 Mb of cDNA.

### DETAILED DESCRIPTION OF THE INVENTION

The present invention relates to altered kinase polypeptides, nucleic acids encoding such polypeptides, cells containing such nucleic acids, antibodies to such polypeptides, assays utilizing such polypeptides, and methods relating to all of the foregoing. The present invention is based upon the identification of mutant kinase polypeptides and kinase variants involved in human malignancies. The polypeptides and nucleic acids of the invention may be produced using well-known and standard synthesis techniques when given the sequences presented herein.

The term “nucleic acid molecule” as used herein refers to any nucleic acid in any possible configuration, such as single stranded, double stranded or a combination thereof. Nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA) and tecto-RNA molecules (e.g. Liu, B., et al., J. Am. Chem. Soc. 126, 4076-4077 (2004)). LNA has a modified RNA backbone with a methylene bridge between C4′ and O2′, providing the respective molecule with a higher duplex stability and nuclease resistance. DNA or RNA may be of genomic or synthetic origin. A respective nucleic acid may furthermore contain non-natural nucleotide analogues and/or be linked to an affinity tag or a label.

Many nucleotide analogues are known and can be used in nucleic acids used in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

### I. The Nucleic Acids of the Invention

As mentioned above, the invention also relates to nucleic acid molecules that encode a mutant protein kinase polypeptide. In some embodiments the mutant protein kinase polypeptide is one of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70. These mutant kinases include one or more of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T17I, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V.

In another aspect, the invention is directed to a nucleic acid molecule encoding a protein kinase polypeptide variant. In some embodiments the protein kinase polypeptide variant is one of AATYK (AATK), ACK1, AXL, CCK4, EPHA1, EPHA2, EPHA3, EPHB3, FAK, FES, HER2, LMTK2 (AATYK2/BREK), MATK, MER, NTRK3, PDGRFA, PDGFRB, PTK-9, PYK2, RON, ROS, RYK, TEK, TNK1, TXK, TYK2, VEGFR1, VEGFR2, VEGFR3, and ZAP70 and includes at least one of the germline alterations AATYK G600C, AATYK G641S, AATYK F1163S, AATYK T1227M, ACK1 P725L, AXL G517S, CCK4 P693L, CCK4 A777V, CCK4 S795R, EPHA1 S936L, EPHA2 R876H, EPHA3 I564V, EPHB3 R514Q, FAK L926delinsPWRL, FES P397R, FES S72_K129del, FES E413fsX131, HER2 R1161Q, LMTK2 59101, MATK A496T, MER E823Q, NTRK3 E402_F410delinsV, NTRK3 G466_Y529delinsD, NTRK3 R711_V712ins16, PDGFRA L221F, PDGFRA S478P, PDGFRB T464M, PTK-9 E195_V196insRPEDHIG, PYK2 G414V, RON Q473_D515del, RON R627fsX23, RON R813_C814insQ, ROS C76fsX, RYK F516L, TEK V600L, TNK1 D472_R473del, TNK1 M598fsX5, TXK R63C, TXK Y414fsX15, TYK2 E971fsX67, VEGFR1 Y642H, VEGFR1 E982A, VEGFR1 P1201L, VEGFR2 C482R, VEGFR3 R1321Q and ZAP70 K186fsX.

In certain aspects of the invention, the nucleic acid molecules may be isolated, enriched, or purified. The mutant kinase polypeptide encoded by said nucleic acid molecules may include, consist essentially of or consist of the amino acid sequence set forth in SEQ ID Nos: 1-256, 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641. Also included are nucleic acids encoding mutant kinase polypeptide fragments of said amino acid sequences set forth in SEQ ID Nos: 1-256, 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641, as long as the mutation or mutated region is retained. In some embodiments, these fragments are at least 10, at least 15, at least 20, at least 30 or at least 35 amino acids long.

By “isolated” in reference to a nucleic acid is meant a polymer of nucleotides conjugated to each other, including DNA and RNA, that is isolated from a natural source or that is synthesized. The isolated nucleic acids of the present invention are not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2-5 fold) of the total DNA or RNA present in the cells or solution of interest than in normal or diseased cells or in the cells from which the sequence was taken. This could be achieved by reducing the amount of other DNA or RNA present, or by increasing the amount of the specific DNA or RNA sequence, or by a combination of the two. However, it should be noted that being enriched does not imply that there are no other DNA or RNA sequences present, it merely defines that the relative amount of the sequence of interest has been significantly increased.

The term “significant” is used to indicate that the level of increase is useful to the person making such an increase, and generally means an increase relative to other nucleic acids of about at least about 2 fold, such as at least about 5 to about 10 fold or even more. The term does also not imply that there is no DNA or RNA from other sources present. As an illustrative example, another source of DNA may, for example, include a yeast or bacterial genome, or a cloning vector. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be present in purified form. The term “purified” in reference to nucleic acid does not require absolute purity (such as a homogeneous preparation). Instead, it represents an indication that the sequence is relatively more pure than in the natural environment (compared to the natural level this level should be at least 2-5 fold greater, e.g., in terms of mg/ml). Individual clones isolated from a cDNA library may be purified to electrophoretic homogeneity. The claimed DNA molecules obtained from these clones could be obtained directly from total DNA or from total RNA. The cDNA clones are not naturally occurring, rather they are typically obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, including two or three orders, and more, such as four or five orders of magnitude is expressly contemplated.

By a “mutant kinase polypeptide” as used herein is meant a protein kinase polypeptide including a somatic mutation. Such a mutation may be a deletion, insertion or substitution of one or more amino acids. In some embodiments, the term refers to a contiguous sequence of at least about 50, such as about 100, about 200, or about 300 amino acids set forth in the amino acid sequence of SEQ ID Nos: 1-256, or the corresponding full-length amino acid sequence, with the proviso that the desired mutation is included in said amino acid sequence. In case the mutation leads to a premature stop codon in the nucleotide sequence encoding the mutant kinase polypeptide, the sequence may even be shorter than the above 50 amino acids. The kinase polypeptide can be encoded by a full-length nucleic acid sequence, i.e. the complete coding sequence of the respective gene, or any portion of the full-length nucleic acid sequence, as long as the mutation of the polypeptide is retained.

The amino acid sequences will be substantially similar to the sequences shown in SEQ ID Nos: 1-256, or to fragments thereof. A sequence that is substantially similar to any one of the sequences of SEQ ID Nos: 1-256 or fragment thereof will in some embodiments have at least about 80, such as at least about 90% identity (in some embodiments at least about 95% or 99-100%) to the sequence of SEQ ID Nos: 1-256, with the proviso that the desired mutation is retained.

The term “kinase variant” or “protein kinase polypeptide variant” relates to a kinase polypeptide that includes a germline alteration. Such an alteration may be a deletion, insertion or substitution of one or more amino acids, and may include single nucleotide polymorphisms (SNPs). While such alterations may themselves not be pathological, they may play a role in the predisposition, development and progression of proliferative diseases and disorders, for example human malignancies. In the context of the present invention, the term ‘kinase variant’ in some embodiments refers to a contiguous sequence of at least about 50, such as about 100, about 200, or about 300 amino acids set forth in the amino acid sequence of SEQ ID Nos: 513-642, or the corresponding full-length amino acid sequence, with the proviso that said alteration is included in said amino acid sequence. In case the mutation leads to a premature stop codon in the nucleotide sequence encoding the kinase variant, the sequence may even be shorter than the above 50 amino acids. The kinase polypeptide can be encoded by a full-length nucleic acid sequence, i.e. the complete coding sequence of the respective gene, or any portion of the full-length nucleic acid sequence, as long as the alteration of the polypeptide is retained.

The amino acid sequences will be substantially similar to the sequences shown in SEQ ID Nos: 513-642 or to fragments thereof. A sequence that is substantially similar to any one of the sequences of SEQ ID Nos: 513-642 or fragment thereof will in some embodiments have at least 80, such as at least 90% identity (in some embodiments at least 95% or 99-100%) to the sequence of SEQ ID Nos: 513-642, with the proviso that the altered position or sequence is retained.

By “identity” is meant a property of sequences that measures their similarity or relationship. Identity is measured by dividing the number of identical residues by the total number of residues and gaps and multiplying the product by 100.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of identity. Those skilled in the art will recognize that several computer programs are available for determining sequence identity using standard parameters, for example Blast (Altschul, et al. (1997) Nucleic Acids Res. 25:3389-3402), Blast2 (Altschul, et al. (1990) J. Mol. Biol. 215:403-410), and Smith-Waterman (Smith, et al. (1981) J. Mol. Biol. 147:195-197).

The term “mutated” or “mutant” in reference to a nucleic acid or a polypeptide refers to the exchange, deletion, or insertion of one or more nucleotides or amino acids, respectively, compared to the naturally occurring nucleic acid or polypeptide.

The term “altered” or “variant” in reference to a nucleic acid or polypeptide refers to polymorphisms, i.e. the exchange, deletion, or insertion of one or more nucleotides or amino acids, respectively, compared to the predominant form of the respective nucleic acid or polypeptide.

Also encompassed by the present invention are nucleic acid molecules substantially complementary to the above nucleic acid molecules. “Substantially complementary” as used herein refers to the fact that a given nucleic acid molecule is at least 90, at least 95, or 99 or 100% complementary to another nucleic acid. The term “complementary” or “complement” refers to two nucleotides that can form multiple favorable interactions with one another. Such favorable interactions include Watson-Crick base pairing. A nucleotide sequence is the complement of another nucleotide sequence if all of the nucleotides of the first sequence are complementary to all of the nucleotides of the second sequence.

The nucleic acids according to the invention may be isolated from a natural source by cDNA cloning or subtractive hybridization or other routine techniques known to a person skilled in the art. The natural source may be any organism. As an illustrative example, the nucleic acids may be isolated from a mammalian source. It may for example be of human origin. The natural source can include blood, semen, or tissue.

The term “mammalian” refers to any mammal, for example, species such as mice, rats, rabbits, guinea pigs, sheep, and goats, cats, dogs, monkeys, apes, and humans.

The nucleic acids of the invention may also be synthetic, meaning being synthesized by the triester method or by using an automated DNA synthesizer.

The above nucleic acid molecules of the invention encoding mutant kinase polypeptides, may further include a vector or promoter effective to initiate transcription in a host cell. The recombinant nucleic acid can alternatively contain a transcriptional initiation region functional in a cell, a sequence complementary to an RNA sequence encoding a kinase polypeptide and a transcriptional termination region functional in a cell. Specific vectors and host cell combinations are discussed herein. Thus, the present invention also encompasses nucleic acids of recombinant origin, such as a cell or an organism.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a kinase can be inserted into a vector by cutting the vector with restriction enzymes and ligating the two pieces together.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

The nucleic acids according to the invention may include, consist essentially of or consist of the nucleotide sequence set forth in any one of SEQ ID Nos: 257-512. Alternatively, the nucleic acids of the invention may also include, consists essentially of or consist of the nucleotide sequence set forth in any one of SEQ ID Nos: 643-646, 649, 654-655, 657-658, 663, 667-668, 673, 677-680, 692, 701-703, 713-717, 719-721, 728, 730-731, 737, 746, 750-751, 753-754, 756, 760, 762-764, 767, and 770-771.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the kinase genes of the invention could be synthesized to give a nucleic acid sequence significantly different from that shown in SEQ ID Nos: 257-512, 643-646, 649, 654-655, 657-658, 663, 667-668, 673, 677-680, 692, 701-703, 713-717, 719-721, 728, 730-731, 737, 746, 750-751, 753-754, 756, 760, 762-764, 767, and 770-771. The encoded amino acid sequence thereof would, however, be preserved.

In addition, the nucleic acid sequence may include a nucleotide sequence which results from the addition, deletion or substitution of at least one nucleotide to the 5′-end and/or the 3′-end of the nucleic acid formula shown in SEQ ID Nos: 257-512, 643-646, 649, 654-655, 657-658, 663, 667-668, 673, 677-680, 692, 701-703, 713-717, 719-721, 728, 730-731, 737, 746, 750-751, 753-754, 756, 760, 762-764, 767, and 770-771, or a derivative thereof. Any nucleotide or polynucleotide may be used in this regard, provided that its addition, deletion or substitution does not alter the amino acid sequence of SEQ ID Nos: 1-256, 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641, which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the kinase genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

### II. Nucleic Acid Probes, Methods, and Kits for the Detection of Mutant Kinases.

The nucleic acid molecules of the invention are also useful for the design of hybridization probes to facilitate identification and cloning of mutated kinase polypeptides or kinase variants, the design of PCR probes to facilitate cloning of mutated kinase polypeptides or kinase variants, obtaining antibodies directed against mutated kinase polypeptides and kinase variants, and designing antisense oligonucleotides.

Therefore, the invention is also directed to nucleic acid probes for the detection of nucleic acid molecules encoding a mutant kinase polypeptide or a kinase polypeptide variant in a sample.

The mutant kinase polypeptide may in some embodiments be selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, and may include at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N2671, BRK W78fsX58, BTK M489I, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V.

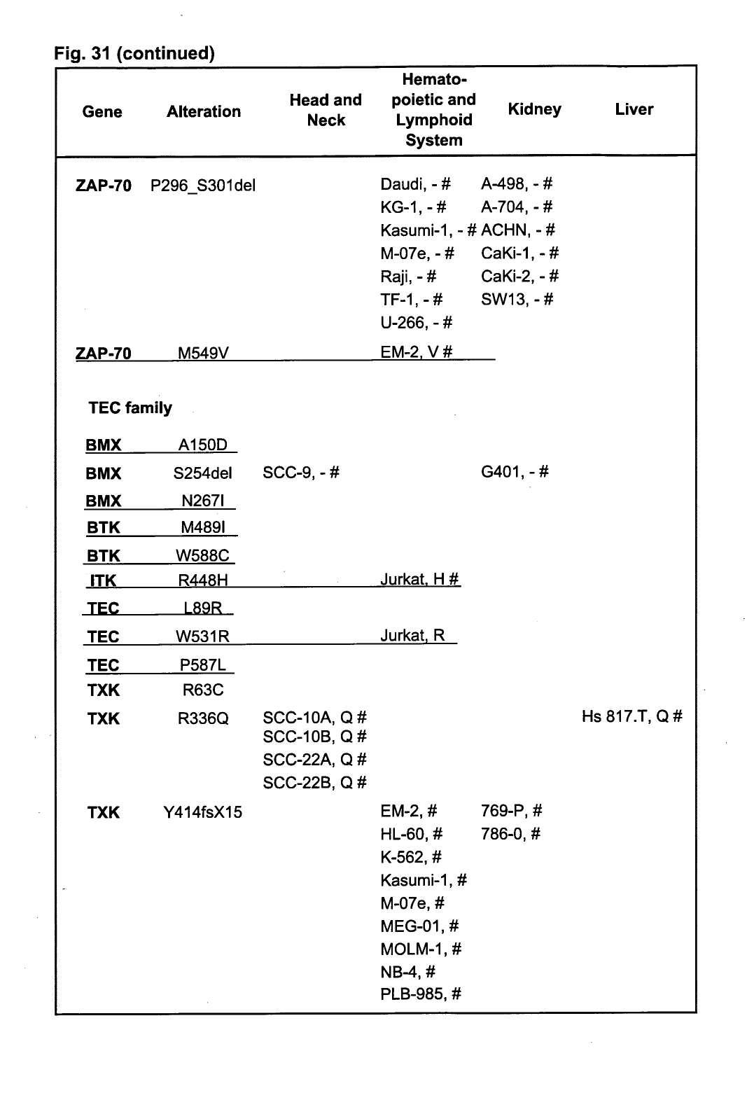

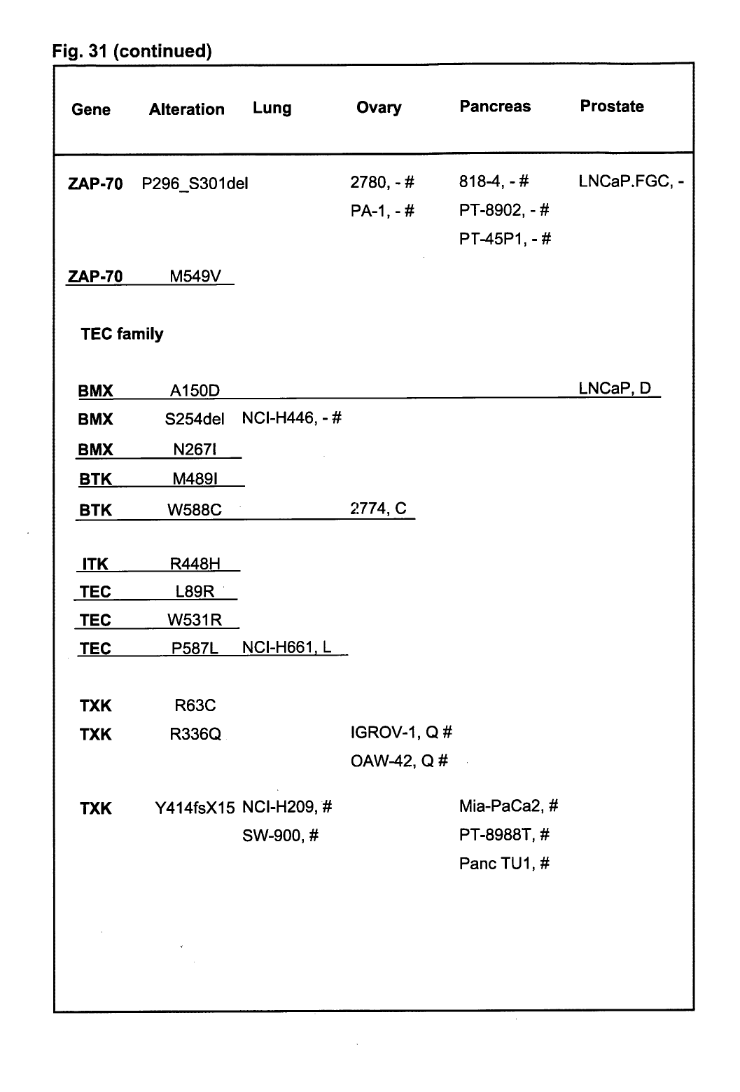

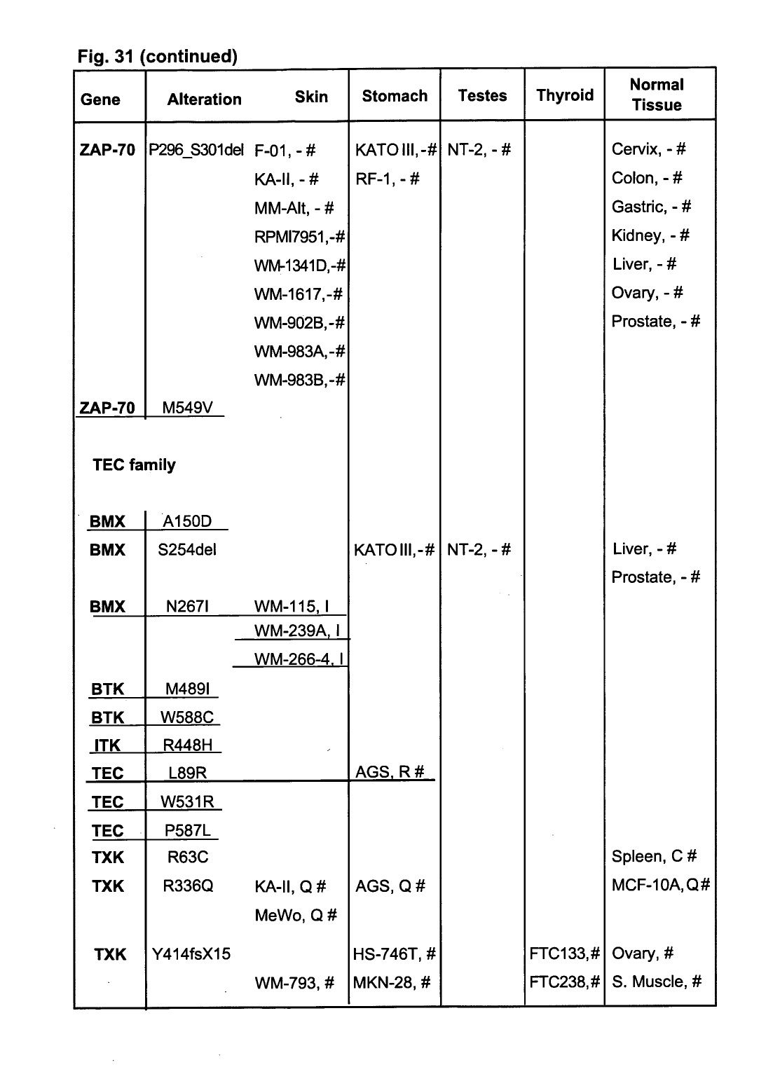

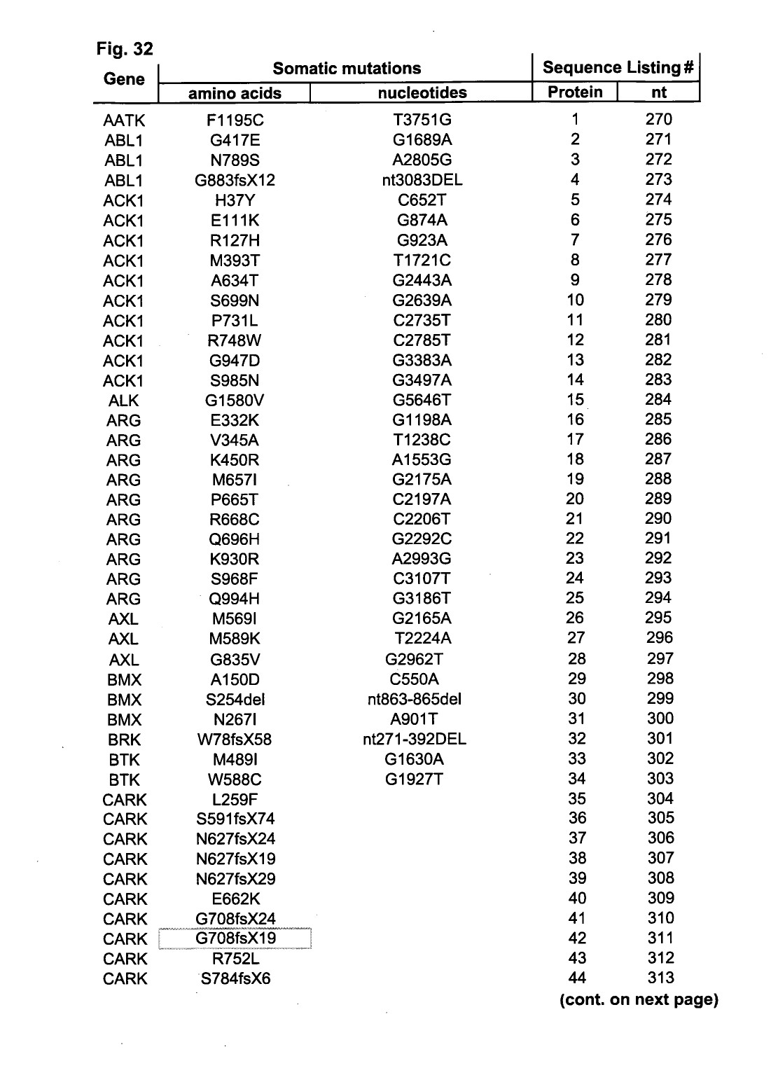

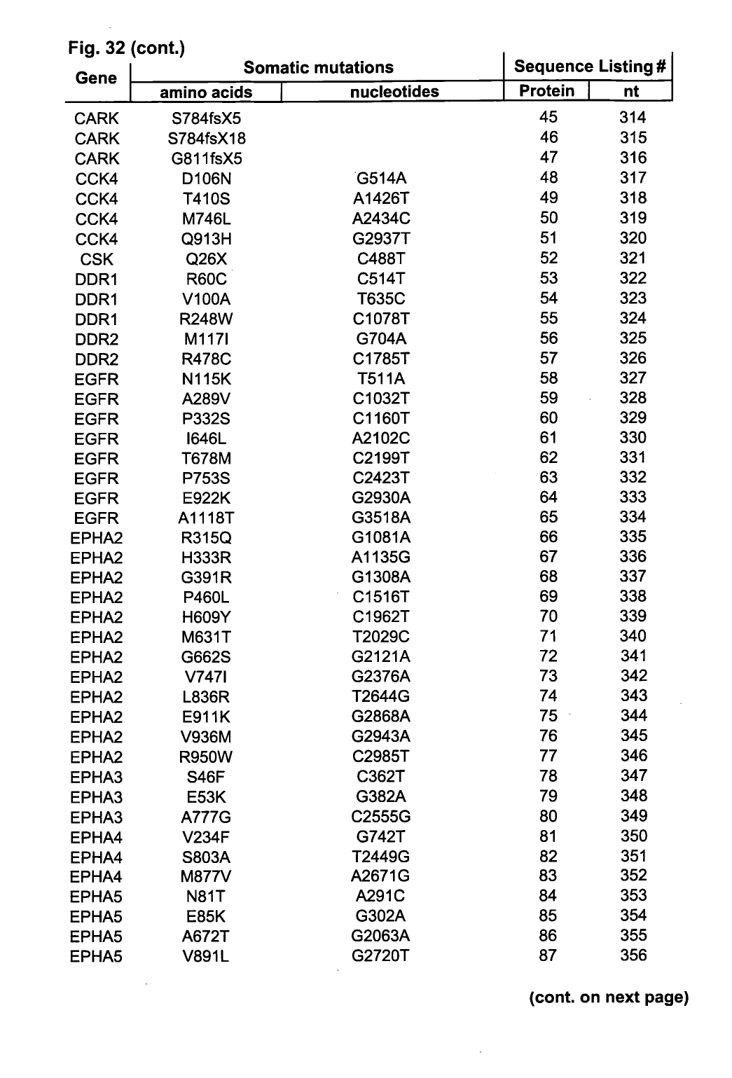

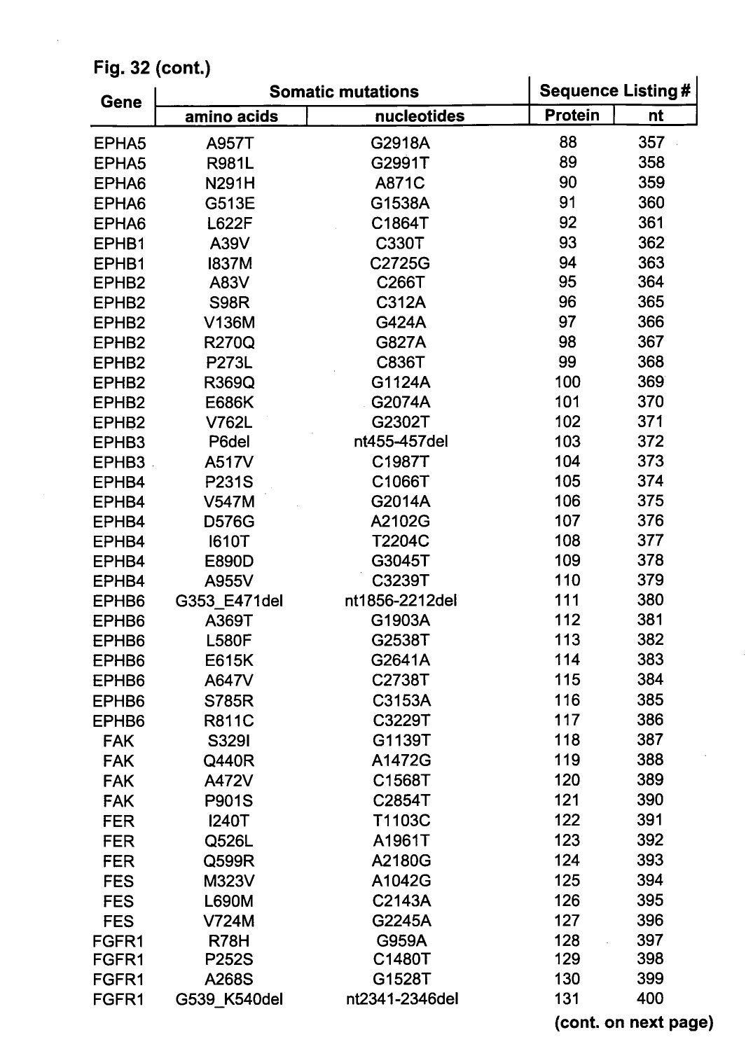

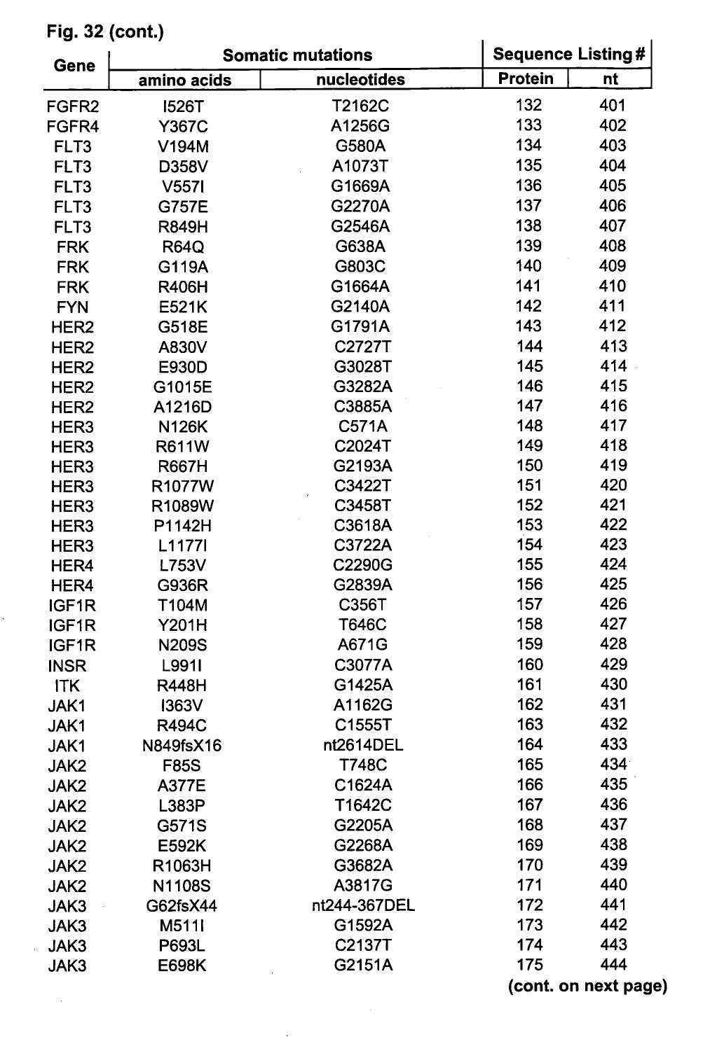

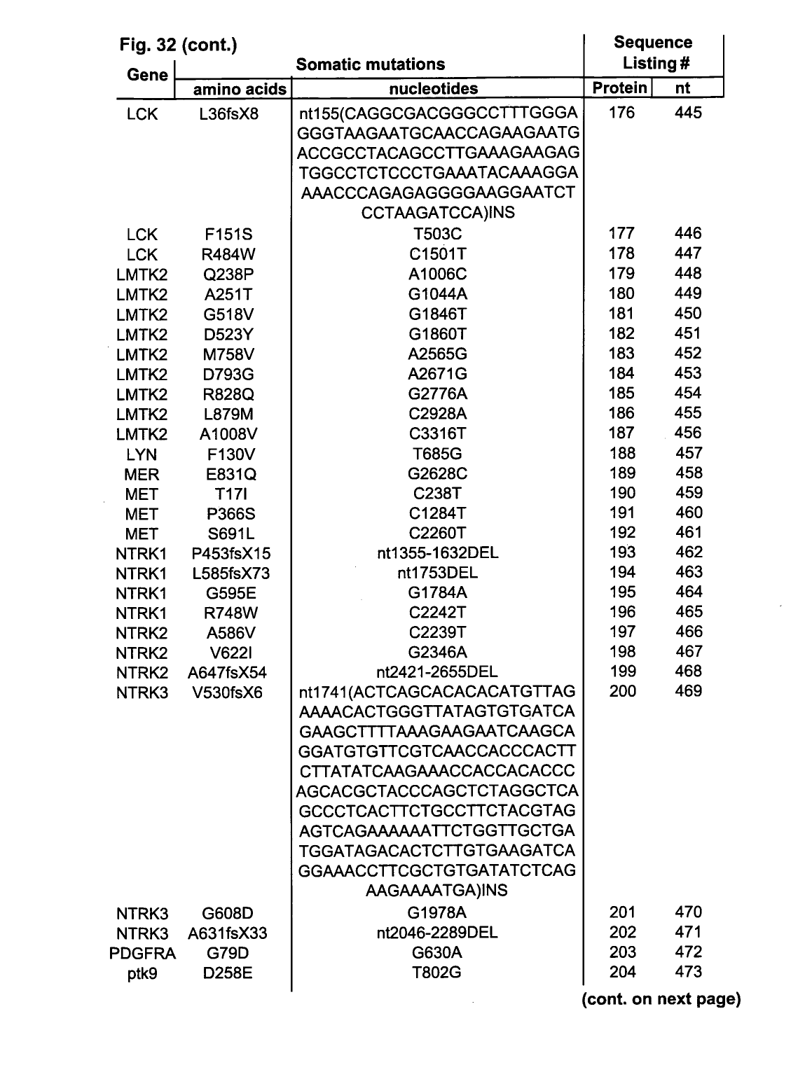

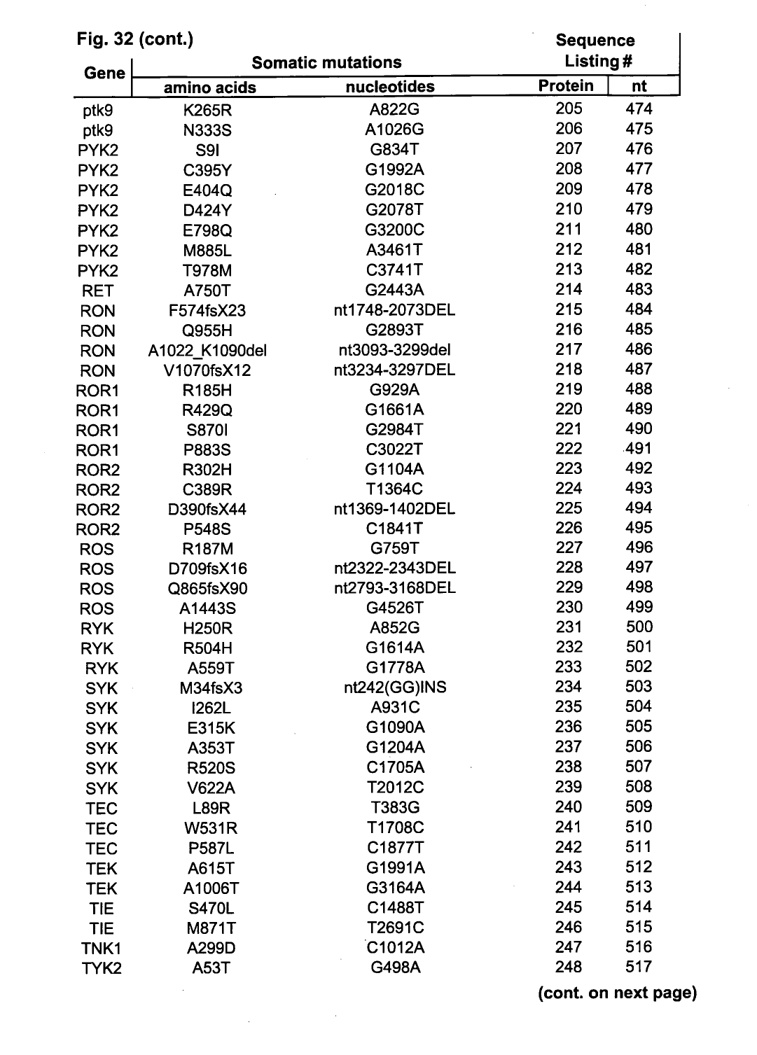

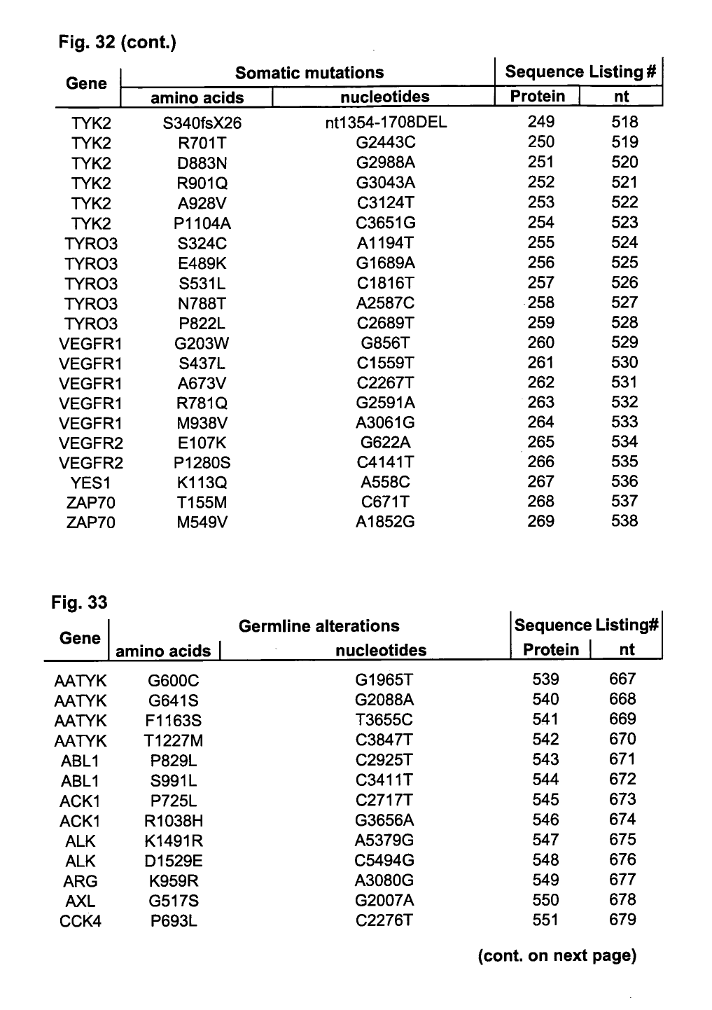

The nucleic acid probes of the invention may include, consist essentially of or consist of nucleotide sequences that will hybridize to a target region in the nucleic acid sequence set forth in any of SEQ ID Nos: 257-512, or a functional equivalent thereof. The target region the nucleic acid probes of the invention are binding to include the mutation or mutated region, indicated in FIG. 32.

In another embodiment, the nucleic acid probe may be suitable for the detection of kinase variants selected from the group of AATYK (AATK), ACK1, AXL, CCK4, EPHA1, EPHA2, EPHA3, EPHB3, FAK, FES, HER2, LMTK2 (AATYK2/BREK), MATK, MER, NTRK3, PDGRFA, PDGFRB, PTK-9, PYK2, RON, ROS, RYK, TEK, TNK1, TXK, TYK2, VEGFR1, VEGFR2, VEGFR3, and ZAP70 and including at least one of the germline alterations AATYK G600C, AATYK G641S, AATYK F1163S, AATYK T1227M, ACK1 P725L, AXL G517S, CCK4 P693L, CCK4 A777V, CCK4 S795R, EPHA1 S936L, EPHA2 R876H, EPHA3 I564V, EPHB3 R514Q, FAK L926delinsPWRL, FES P397R, FES S72_K129del, FES E413fsX131, HER2 R1161Q, LMTK2 S910I, MATK A496T, MER E823Q, NTRK3 E402_F410delinsV, NTRK3 G466_Y529delinsD, NTRK3 R711_V712ins16, PDGFRA L221F, PDGFRA S478P, PDGFRB T464M, PTK-9 E195_V196insRPEDHIG, PYK2 G414V, RON Q473_D515del, RON R627fsX23, RON R813_C814insQ, ROS C76fsX, RYK F516L, TEK V600L, TNK1 D472_R473del, TNK1 M598fsX5, TXK R63C, TXK Y414fsX15, TYK2 E971fsX67, VEGFR1 Y642H, VEGFR1 E982A, VEGFR1 P1201L, VEGFR2 C482R, VEGFR3 R1321Q and ZAP70 K186fsX.

The kinase “target region” is the nucleotide base sequence set forth in SEQ ID Nos: 257-512, 643-646, 649, 654-655, 657-658, 663, 667-668, 673, 677-680, 692, 701-703, 713-717, 719-721, 728, 730-731, 737, 746, 750-751, 753-754, 756, 760, 762-764, 767, and 770-771 or the corresponding full-length sequences, a functional derivative thereof, or a fragment thereof to which the nucleic acid probe will specifically hybridize, as long as said nucleotide base sequence includes any one of the above indicated mutations or alterations. Specific hybridization indicates that in the presence of other nucleic acids the probe only hybridizes detectably with the target region of the mutant kinase or kinase variant of the invention.

A nucleic acid probe of the present invention may be used to probe a sample or a chromosomal/cDNA library by usual hybridization methods to detect the presence of nucleic acid molecules of the present invention. A chromosomal DNA or cDNA library may be prepared from appropriate cells according to methods well established in the art.

In order to obtain nucleic acid probes having nucleotide sequences which correspond to altered portions of the amino acid sequence of the polypeptide of interest, chemical synthesis can be carried out. The synthesized nucleic acid probes may be first used as primers in a polymerase chain reaction (PCR) carried out in accordance with recognized PCR techniques, essentially according to standard PCR Protocols utilizing the appropriate template, in order to obtain the probes of the present invention.

One skilled in the art will readily be able to design such probes based on the sequence disclosed herein using methods of computer alignment and sequence analysis well known in the art. The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

One method of detecting the presence of nucleic acids of the invention in a sample includes (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

The above method may also utilize a set of the nucleic acid probes of the invention to simultaneously detect the presence of the nucleic acids of the invention in a sample. Such a method may be useful for the diagnosis of proliferative diseases or disorders in a subject and may also be useful to predict the risk of cancer with high predictive accuracy and/or to choose an adequate therapy.

The set of nucleic acid probes utilized in such a method of the invention may be chosen in view of the condition to be detected and may include nucleic acid probes for all or any subset of the nucleic acid molecules that are implicated by the present invention in the predisposition, development and progression of cancer, including the nucleotide sequences set forth in SEQ ID Nos: 257-512 and 643-772. Such a subset may include at least 2, for example at least 5, 7, 10, 12, 16, 20, 30, 40, 50, 60, 70, 80, 90, 100, 110, 120, 150, 200, 250, 300, 350 or any other number, for example all of the above sequences.

For the above method one or more nucleic acid probes may be bound to or immobilized on a solid support. Said solid support may be a chip, for example a DNA microchip.

A kit for detecting the presence of nucleic acids of the invention in a sample includes at least one container means having disposed therein the above-described nucleic acid probe. The kit may further include other containers that include one or more of the following: wash reagents and reagents capable of detecting the presence of bound nucleic acid probe. Examples of detection reagents include, but are not limited to radiolabeled probes, enzymatic labeled probes (horseradish peroxidase, alkaline phosphatase), and affinity labeled probes (biotin, avidin, or steptavidin).

In detail, a compartmentalized kit includes any kit in which reagents are included in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

III. Dna Constructs Including a Nucleic Acid Molecule of the Invention and Cells Containing these Constructs.

The invention further describes a recombinant cell or tissue including a nucleic acid molecule according to the invention, as detailed above.

In such cells, the nucleic acid may be under the control of the genomic regulatory elements, or may be under the control of heterologous regulatory elements including a heterologous promoter.

The term “heterologous” refers to the relationship between two or more nucleic acid or protein sequences that are derived from different sources. For example, a promoter is heterologous with respect to a transcribable polynucleotide sequence if such a combination is not normally found in nature. In addition, a particular sequence may be “heterologous” with respect to a cell or organism in that it encodes a protein or is included in a protein, for example a recombinant protein, that is not normally expressed by the host cell, tissue, or species. Such a heterologous protein accordingly generally is or has been inserted into the respective host cell, tissue, or species. Accordingly, a heterologous promoter is not normally coupled in vivo transcriptionally to the coding sequence for the kinase polypeptides.

Therefore, the present invention also relates to a recombinant DNA molecule including, 5′ to 3′, a promoter effective to initiate transcription in a host cell and the above-described nucleic acid molecules. In addition, the present invention relates to a recombinant DNA molecule including a vector and an above-described nucleic acid molecule. The present invention also relates to a nucleic acid molecule including a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention further relates to a cell or organism that contains an above-described nucleic acid molecule and thereby is capable of expressing a polypeptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a mutant kinase of the invention may be obtained by the above-described methods. This region may be retained for its transcriptional termination regulatory sequences, such as termination and polyadenylation. Thus, by retaining the 3′-region naturally contiguous to the DNA sequence encoding a kinase of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a mutant kinase of the invention) are said to be operably linked if the nature of the linkage between the two DNA sequences does not (1) result in the introduction of a frame-shift mutation, (2) interfere with the ability of the promoter region sequence to direct the transcription of a gene sequence encoding a kinase of the invention, or (3) interfere with the ability of the gene sequence of a kinase of the invention to be transcribed by the promoter region sequence.

Thus, a promoter region would be operably linked to a DNA sequence if the promoter were capable of effecting transcription of that DNA sequence. Thus, to express a gene encoding a mutant kinase of the invention, transcriptional and translational signals recognized by an appropriate host are necessary.

The present invention encompasses the expression of a gene encoding a kinase of the invention (or a functional derivative thereof) in either prokaryotic or eukaryotic cells. Prokaryotic hosts are, generally, very efficient and convenient for the production of recombinant proteins and are, therefore, one type of expression system for mutant kinases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include γgt10, γgt11 and the like; and suitable virus vectors may include pMAM-neo, pKRC and the like. In some embodiments the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a kinase of the invention (or a functional derivative thereof) in a prokaryotic cell, it is necessary to operably link the sequence encoding the kinase of the invention to a functional prokaryotic promoter. Such promoters may be either constitutive or regulatable (i.e., inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, recA, λacZ, λacI, and gal promoters of E. coli, the α-amylase (Ulmanen et al., J. Bacteriol. 162:176-182, 1985) and the ι-28-specific promoters of B. subtilis (Gilman et al., Gene Sequence 32:11-20, 1984), and the promoters of the bacteriophages of Bacillus, and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the kinase polypeptide of interest. Suitable hosts may often include eukaryotic cells. Examples of eukaryotic hosts include, but are not limited to, yeast, fungi, insect cells, mammalian cells either in vivo, or in tissue culture. Mammalian cells which may be useful as hosts include for example HeLa cells, cells of fibroblast origin such as VERO or CHO-K1, or cells of lymphoid origin and their derivatives. In some embodiments the mammalian host cells include any, including all human cancer cell lines.

Another suitable host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of kinases of the invention in insect cells (Jasny, Science 238:1653, 1987).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids, which can be utilized for production of the desired proteins in yeast. Yeast recognizes leader sequences on cloned mammalian genes and secretes peptides bearing leader sequences (i.e., pre-peptides). Several possible vector systems are available for the expression of kinases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of mutant kinases of the invention in eukaryotic hosts requires the use of eukaryotic regulatory regions. Such regions will, in general, include a promoter region sufficient to direct the initiation of RNA synthesis. Examples of a suitable eukaryotic promoter include, but are not limited to, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it may be desired to ensure that the linkage between a eukaryotic promoter and a DNA sequence which encodes a kinase of the invention (or a functional derivative thereof) does not contain any intervening codons which are capable of encoding a methionine (i.e., AUG). The presence of such codons results either in the formation of a fusion protein (if the AUG codon is in the same reading frame as the kinase of the invention coding sequence) or a frame-shift mutation (if the AUG codon is not in the same reading frame as the kinase of the invention coding sequence).

A nucleic acid molecule encoding a kinase of the invention and an operably linked promoter may be introduced into a recipient prokaryotic or eukaryotic cell either as a nonreplicating DNA or RNA molecule, which may either be a linear molecule ora closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

An illustrative example of a prokaryotic vector is a plasmid, such as a plasmid capable of replication in E. coli (such as, for example, pBR322, ColE1, pSC101, pACYC 184, πVX). Bacillus plasmids include pC194, pC221, pT127, and the like. Suitable Streptomyces plasmids include p1J101 (Kendall et al., J. Bacteriol. 169:4177-4183, 1987), and streptomyces bacteriophages such as φC31. Pseudomonas plasmids are reviewed by John et al. (Rev. Infect. Dis. 8:693-704, 1986), and Izaki (Jpn. J. Bacteriol. 33:729-742, 1978).

Examples of an eukaryotic plasmid include, but are not limited to, BPV, vaccinia, SV40, 2-micron circle, and the like, or their derivatives. Such plasmids are well known in the art (e.g. Broach, Cell 28:203-204, 1982; Bollon et al., J. Clin. Hematol. Oncol. 10:39-48, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a kinase of the invention, or fragments thereof. This can take place in the transformed cells as such, or following the induction of these cells to differentiate. A variety of incubation conditions can be used to form the peptide of the present invention. It may be desired to use conditions thatmimic physiological conditions.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

### IV. Proteins of the Invention

The mutant kinase polypeptides of the invention are selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70, and include at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M6571, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M4891, BTK W588C, CCK4 D106N, CCK4 T410S, CCK4 M746L, CCK4 Q913H, CSK Q26X, DDR1 R60C, DDR1 V100A, DDR1 R248W, DDR2 M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 5803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S3291, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V557I, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L9911, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S9I, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V and may be isolated, enriched or purified.

Also included are kinase variants that are selected from the group consisting of AATYK (AATK), ACK1, AXL, CCK4, EPHA1, EPHA2, EPHA3, EPHB3, FAK, FES, HER2, LMTK2 (AATYK2/BREK), MATK, MER, NTRK3, PDGRFA, PDGFRB, PTK-9, PYK2, RON, ROS, RYK, TEK, TNK1, TXK, TYK2, VEGFR1, VEGFR2, VEGFR3, and ZAP70 and including at least one of the germline alterations AATYK G600C, AATYK G641S, AATYK F1163S, AATYK T1227M, ACK1 P725L, AXL G517S, CCK4 P693L, CCK4 A777V, CCK4 S795R, EPHA1 S936L, EPHA2 R876H, EPHA3 I564V, EPHB3 R514Q, FAK L926delinsPWRL, FES P397R, FES S72_K129del, FES E413fsX131, HER2 R1161Q, LMTK2 S910I, MATK A496T, MER E823Q, NTRK3 E402_F410delinsV, NTRK3 G466_Y529delinsD, NTRK3 R711_V712ins16, PDGFRA L221F, PDGFRA S478P, PDGFRB T464M, PTK-9 E195_V196insRPEDHIG, PYK2 G414V, RON Q473_D515del, RON R627fsX23, RON R813_C814insQ, ROS C76fsX, RYK F516L, TEK V600L, TNK1 D472_R473del, TNK1 M598fsX5, TXK R63C, TXK Y414fsX15, TYK2 E971fsX67, VEGFR1 Y642H, VEGFR1 E982A, VEGFR1 P1201L, VEGFR2 C482R, VEGFR3 R1321Q and ZAP70 K186fsX. These kinase variants may also be isolated, enriched or purified.

By “isolated” in reference to a polypeptide is meant a polymer of amino acids (2 or more amino acids) conjugated to each other, including polypeptides that are isolated from a natural source or that are synthesized. The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid material naturally associated with it.

By the use of the term “enriched” in reference to a polypeptide is meant that the specific amino acid sequence constitutes a significantly higher fraction (2-5 fold) of the total amino acid sequences present in the cells or solution of interest than in normal or diseased cells or in the cells from which the sequence was taken. This could be caused by preferential reduction in the amount of other amino acid sequences present, or by a preferential increase in the amount of the specific amino acid sequence of interest, or by a combination of the two. However, it should be noted that enriched does not imply that there are no other amino acid sequences present. The term merely defines that the relative amount of the sequence of interest has been significantly increased. The term significant here is used to indicate that the level of increase is useful to the person making such an increase, and generally means an increase relative to other amino acid sequences of about at least 2-fold, for example at least about 5- to 10-fold or even more. The term also does not imply that there is no amino acid sequence from other sources. The other source of amino acid sequences may, for example, include amino acid sequence encoded by a yeast or bacterial genome, or a cloning vector. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-5 fold greater (e.g., in terms of mg/ml). Purification of at least one order of magnitude, such as about two or three orders, including for example about four or five orders of magnitude is expressly contemplated. It may be desired to obtain the substance at least essentially free of contamination at a functionally significant level, for example about 90%, about 95%, or 99% pure.

Explicitly falling within the scope of the present invention are fragments of mutant kinase polypeptides with any one of the amino acid sequences set forth in SEQ ID Nos: 1-256, or the corresponding full-length amino acid sequences thereof, as long as said fragments include one of the mutations set forth in FIG. 32. The mutant kinase polypeptide fragments contain at least 30, 35, 40, 45, 50, 60, 100, 200, or 300 contiguous amino acids of SEQ ID Nos: 1-256, provided that the mutation of interest is included in said protein fragment.

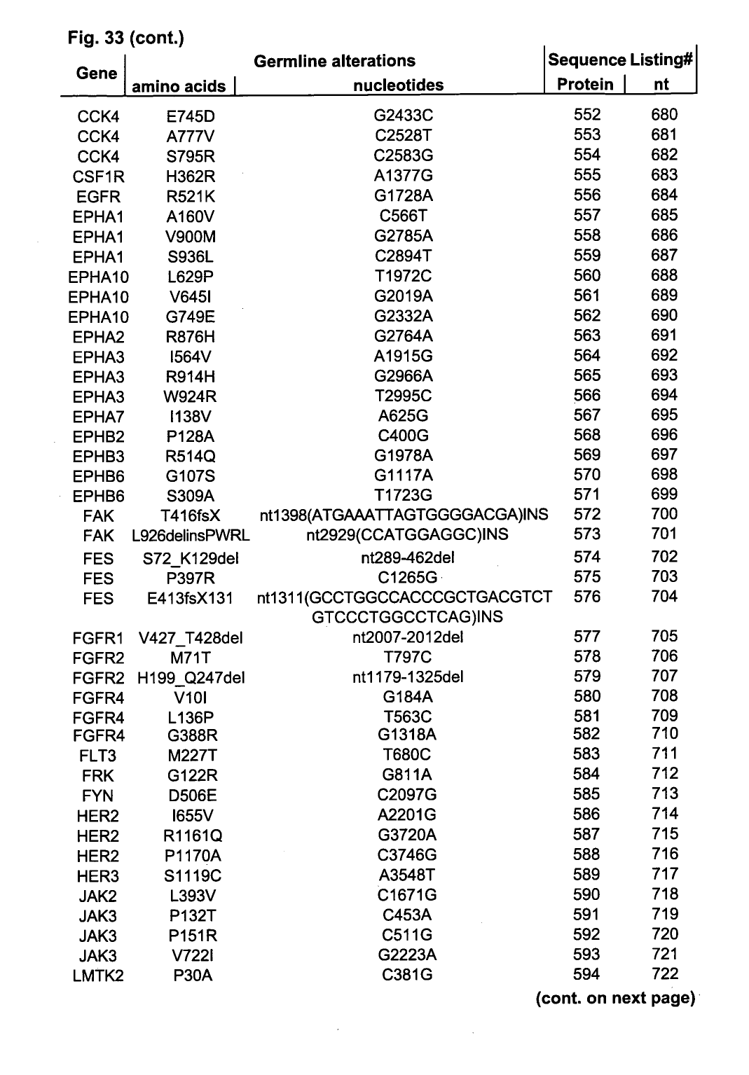

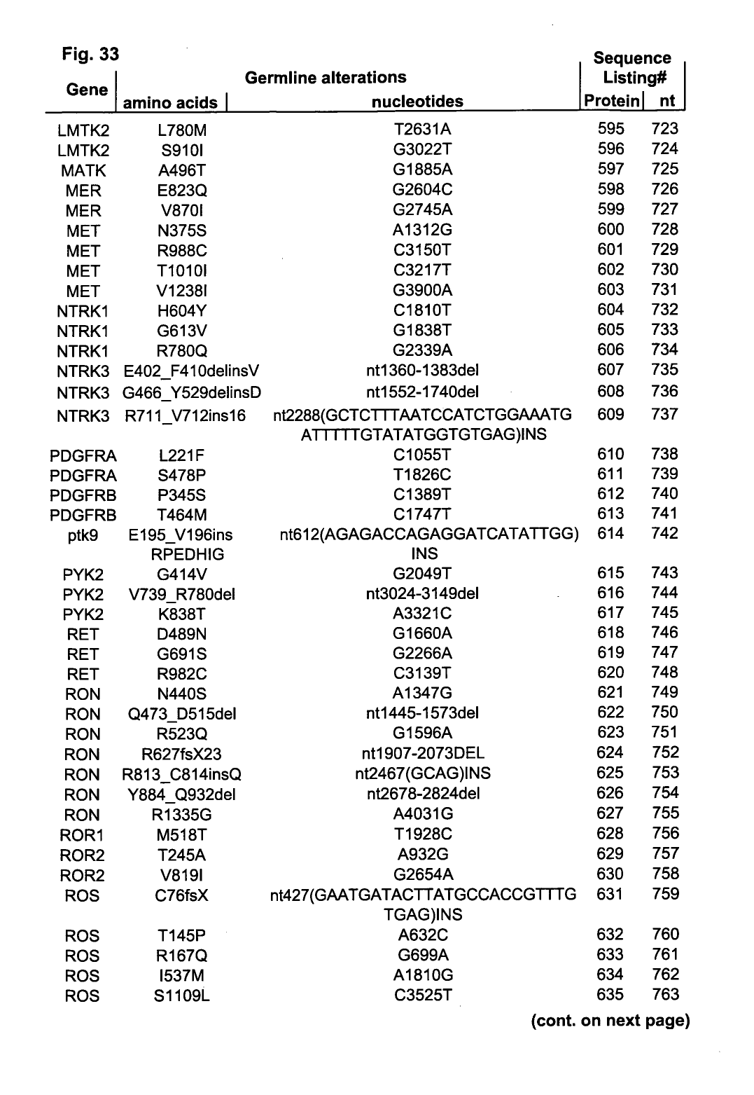

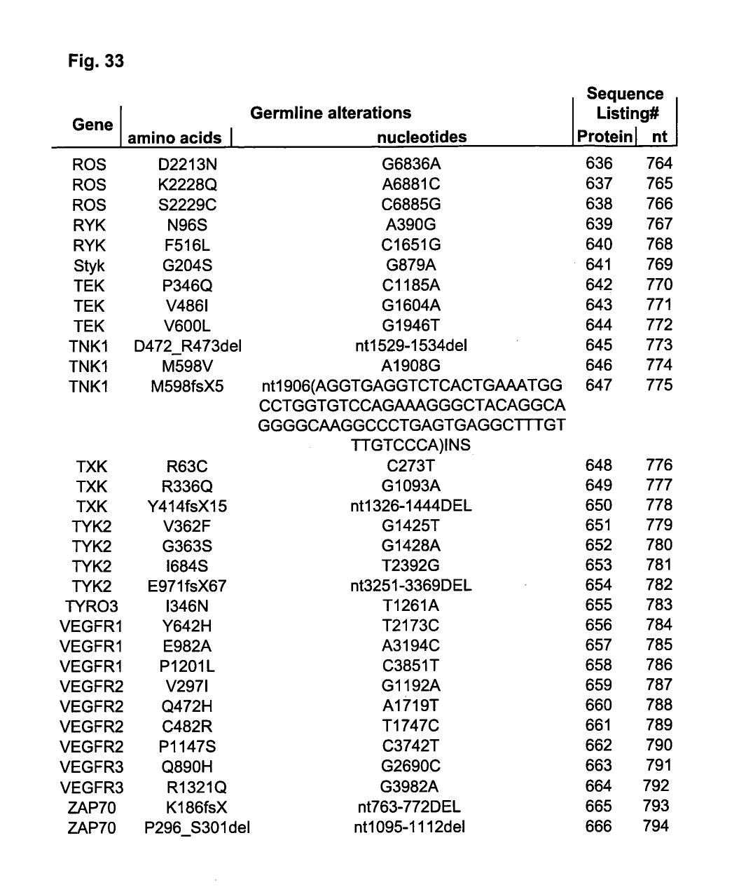

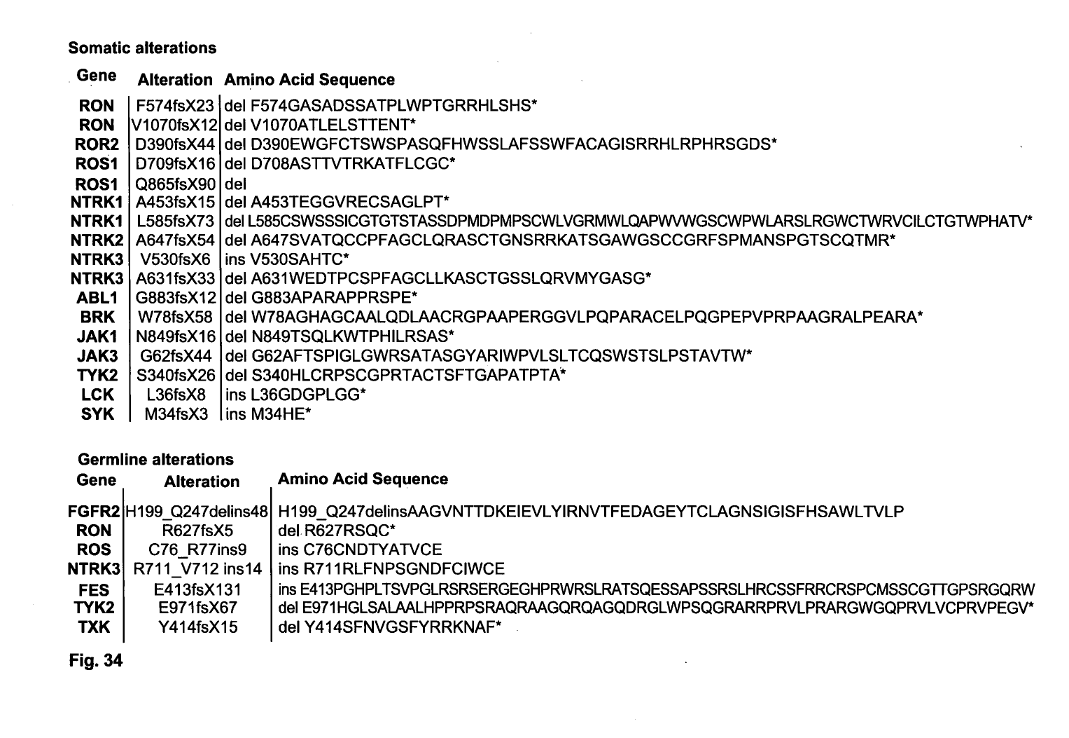

Also encompassed by the present invention are kinase variants with any one of the amino acid sequences set forth in SEQ ID Nos: 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641 and fragments thereof, as long as said fragment include the alteration indicated in FIG. 33. Such fragments may have a length of at least 30, 35, 40, 45, 50, 60, 100, 200, or 300 contiguous amino acids of SEQ ID Nos: 513-516, 519, 524-525, 527-528, 533, 537-538, 543, 547-550, 562, 571-573, 583-587, 589-591, 598, 600-601, 607, 616, 620-621, 623-624, 626, 630, 632-634, 637, and 640-641 with the proviso that said fragment includes the altered sequence position or region.

In case a mutation or polymorphism leads to a premature stop codon, the mutated or altered kinase may be even shorter than 30 amino acids. However, such mutants and variants are also considered to fall within the scope of the present invention.

Also intended to fall within the scope of the present invention, are splice variants of the above mutant kinase polypeptides. Said splice variants may significantly differ from the above amino acid sequences, however, the functional domain architecture, for example the kinase domain, as well as the mutated region have to be retained in such variants. Such splice variants may lead to isoforms of the mutated kinase or kinase variant and may differ from the known form, for example, by an extended or shortened C- or N-terminus or the insertion or deletion of an amino acid sequence stretch. However, the sequence homology and sequence identity between the splice variants/isoforms is sufficiently high so that the skilled person is readily aware that the kinase in question is a mere isoform and not another kinase of the same family. Due to differing lengths of the isoforms, the mutated or altered position may be conserved but the numbering may be changed.

By “fragment” in reference to a polypeptide is meant any amino acid sequence present in a kinase polypeptide, as long as it is shorter than the full length sequence and includes the alteration to be detected.

A variety of methodologies known in the art can be utilized to obtain the polypeptides of the present invention. The polypeptides may be purified from tissues or cells that naturally produce the polypeptides. Alternatively, the above-described isolated nucleic acid fragments could be used to express the recombinant kinase polypeptides of the invention in any organism.

By “recombinant kinase polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

Any eukaryotic organism can be used as a source for the polypeptides of the invention, as long as the source organism naturally contains such polypeptides. As used herein, “source organism” refers to the original organism from which the amino acid sequence of the subunit is derived, regardless of the organism the subunit is expressed in and ultimately isolated from.

As a further alternative the polypeptides of the invention may be synthesized using an automated polypeptide synthesizer.

One skilled in the art can readily follow known methods for isolating proteins in order to obtain the polypeptides free of natural contaminants. These include, but are not limited to: size-exclusion chromatography, HPLC, ion-exchange chromatography, and immuno-affinity chromatography.

V. Antibodies, Methods of their Use and Kits for the Detection of Mutant Kinase Polypeptides

Also encompassed by the invention are antibodies having specific binding affinity only for a mutant kinase polypeptide, or domain or fragment thereof, with the mutant kinase polypeptide being selected from the group consisting of AATYK (AATK), ABL1, ACK1, ALK, ARG, AXL, BMX, BRK, BTK, CCK4, CSK, DDR1, DDR2, EGFR, EPHA2, EPHA3, EPHA4, EPHA5, EPHA6, EPHB1, EPHB2, EPHB3, EPHB4, EPHB6, FAK, FER, FES, FGFR1, FGFR2, FGFR4, FLT3, FRK, FYN, HER2, HER3, HER4, IGF1R, INSR, ITK, JAK1, JAK2, JAK3, LCK, LMTK2 (AATYK2/BREK), LYN, MER, MET, NTRK1, NTRK2, NTRK3, PDGRFA, PTK-9, PYK2, RET, RON, ROR1, ROR2, ROS, RYK, SYK, TEC, TEK, TIE, TNK1, TYK2, TYRO3, VEGFR1, VEGFR2, YES1, and ZAP70 and including at least one of the mutations AATYK F1195C, ABL1 G417E, ABL1 N789S, ABL1 G883fsX12, ACK1 H37Y, ACK1 E111K, ACK1 R127H, ACK1 M393T, ACK1 A634T, ACK1 S699N, ACK1 P731L, ACK1 R748W, ACK1 G947D, ACK1 S985N, ALK G1580V, ARG E332K, ARG V345A, ARG K450R, ARG M657I, ARG P665T, ARG R668C, ARG Q696H, ARG K930R, ARG S968F, ARG Q994H, AXL M569I, AXL M589K, AXL G835V, BMX A150D, BMX S254del, BMX N267I, BRK W78fsX58, BTK M4891, BTK W588C, CCK4 D106N, CCK4 T410S, CCK4 M746L, CCK4 Q913H, CSK Q26X, DDR1 R60C, DDR1 V100A, DDR1 R248W, DDR2M117I, DDR2 R478C, EGFR N115K, EGFR A289V, EGFR P332S, EGFR I646L, EGFR T678M, EGFR P753S, EGFR E922K, EGFR A1118T, EPHA2 R315Q, EPHA2 H333R, EPHA2 G391R, EPHA2 P460L, EPHA2 H609Y, EPHA2 M631T, EPHA2 G662S, EPHA2 V747I, EPHA2 L836R, EPHA2 E911K, EPHA2 V936M, EPHA2 R950 W, EPHA3 S46F, EPHA3 E53K, EPHA3 A777G, EPHA4 V234F, EPHA4 S803A, EPHA4 M877V, EPHA5 N81T, EPHA5 E85K, EPHA5 A672T, EPHA5 V891L, EPHA5 A957T, EPHA5 R981L, EPHA6 N291H, EPHA6 G513E, EPHA6 L622F, EPHB1 A39V, EPHB1 I837M, EPHB2 A83V, EPHB2 S98R, EPHB2 V136M, EPHB2 R270Q, EPHB2 P273L, EPHB2 R369Q, EPHB2 E686K, EPHB2 V762L, EPHB3 P6del, EPHB3 A517V, EPHB4 P231S, EPHB4 V547M, EPHB4 D576G, EPHB4 I610T, EPHB4 E890D, EPHB4 A955V, EPHB6 G353_E471del, EPHB6 A369T, EPHB6 L580F, EPHB6 E615K, EPHB6 A647V, EPHB6 S785R, EPHB6 R811C, FAK S329I, FAK Q440R, FAK A472V, FAK P901S, FER I240T, FER Q526L, FER Q599R, FES M323V, FES L690M, FES V724M, FGFR1 R78H, FGFR1 P252S, FGFR1 A268S, FGFR1 G539_K540del, FGFR21526T, FGFR4 Y367C, FLT3 V194M, FLT3 D358V, FLT3 V5571, FLT3 G757E, FLT3 R849H, FRK R64Q, FRK G119A, FRK R406H, FYN E521K, HER2 G518V, HER2 A830V, HER2 E930D, HER2 G1015E, HER2 A1216D, HER3 N126K, HER3 R611W, HER3 R667H, HER3 R1077W, HER3 R1089W, HER3 P1142H, HER3 L1177I, HER4 L753V, HER4 G936R, IGF1R T104M, IGF1R Y201H, IGF1R N209S, INSR L991I, ITK R448H, JAK1 I363V, JAK1 R494C, JAK1 N849fsX16, JAK2 F85S, JAK2 A377E, JAK2 L383P, JAK2 G571S, JAK2 E592K, JAK2 R1063H, JAK2 N1108S, JAK3 G62fsX47, JAK3 M511I, JAK3 P693L, JAK3 E698K, LCK L36fsX8, LCK F151S, LCK R484W, LMTK2 Q238P, LMTK2 A251T, LMTK2 G518V, LMTK2 D523Y, LMTK2 M758V, LMTK2 D793G, LMTK2 R828Q, LMTK2 L879M, LMTK2 A1008V, LYN F130V, MER E831Q, MET T171, MET P366S, MET S691L, NTRK1 P453fsX15, NTRK1 L585fsX73, NTRK1 G595E, NTRK1 R748W, NTRK2 A586V, NTRK2 V622I, NTRK2 A647fsX54, NTRK3 V530fsX6, NTRK3 G608D, NTRK3 A631fsX33, PDGFRA G79D, PTK-9 D258E, PTK-9 K265R, PTK-9 N333S, PYK2 S91, PYK2 C395Y, PYK2 E404Q, PYK2 D424Y, PYK2 E798Q, PYK2 M885L, PYK2 T978M, RET A750T, RON F574fsX23, RON Q955H, RON A1022_K1090del, RON V1070fsX12, ROR1 R185H, ROR1 R429Q, ROR1 S870I, ROR1 P883S, ROR2 R302H, ROR2 C389R, ROR2 D390fsX46, ROR2 P548S, ROS R187M, ROS D709fsX16, ROS Q865fsX90, ROS A1443S, RYK H250R, RYK R504H, RYK A559T, SYK M34fsX3, SYK I262L, SYK E315K, SYK A353T, SYK R520S, SYK V622A, TEC L89R, TEC W531R, TEC P587L, TEK A615T, TEK A1006T, TIE S470L, TIE M871T, TNK1 A299D, TYK2 A53T, TYK2 S340fsX26, TYK2 R701T, TYK2 D883N, TYK2 R901Q, TYK2 A928V, TYK2 P1104A, TYRO3 S324C, TYRO3 E489K, TYRO3 S531L, TYRO3 N788T, TYRO3 P822L, VEGFR1 G203W, VEGFR1 S437L, VEGFR1 A673V, VEGFR1 R781Q, VEGFR1 M938V, VEGFR2 E107K, VEGFR2 P1280S, YES1 K113Q, ZAP70 T155M, and ZAP70 M549V.

Furthermore, also included are antibodies having specific binding affinity only for a kinase variant, or domain or fragment thereof, with the kinase variant being selected from the group consisting of AATYK (AATK), ABL1, ACK1, ALK, ARG, AXL, CCK4, CSFR1, EGFR, EPHA1, EPHA10, EPHA2, EPHA3, EPHA7, EPHB2, EPHB3, EPHB4, EPHB6, FAK, FES, FGFR1, FGFR2, FGFR3, FGFR4, FLT3, FRK, FYN, HER2, HER3, JAK2, JAK3, LMTK2 (AATYK2/BREK), MATK, MER, MET, NTRK1, NTRK2, NTRK3, PDGRFA, PDGFRB, PTK-9, PYK2, RET, RON, ROR1, ROR2, ROS, RYK, STYK, TEK, TNK1, TXK, TYK2, TYRO3, VEGFR1, VEGFR2, VEGFR3 and ZAP70 and including at least one of the alterations AATYK G600C, AATYK G641S, AATYK F1163S, AATYK T1227M, ABL1 P829L, ABL1 S991L, ACK1 P725L, ACK1 R1038H, ALK K1491R, ALK D1529E, ARG K959R, AXL G517S, CCK4 P693L, CCK4 E745D, CCK4 A777V, CCK4 S795R, CSF1R H362R, EGFR R521K, EPHA1 A160V, EPHA1 V900M, EPHA1 S936L, EPHA10 L629P, EPHA10 V645I, EPHA10 G749E, EPHA2 R876H, EPHA3 I564V, EPHA3 R914H, EPHA3 W924R, EPHA7 I138V, EPHB2 P128A, EPHB3 R514Q, EPHB4 P231S, EPHB6 G107S, EPHB6 S309A, FAK T416fsX, FAK L926delinsPWRL, FES P397R, FES S72_K129del, FES E413fsX131, FGFR1 V427_T428del, FGFR2 M71T, FGFR2H199_Q247del, FGFR3 T311_Q422del, FGFR4 V10I, FGFR4 L136P, FGFR4 G388R, FLT3 M227T, FRK G122R, FYN D506E, HER2 I655V, HER2 R1161Q, HER2 P1170A, HER3 S1119C, JAK2 L393V, JAK3 P132T, JAK3 P151R, JAK3 V722I, LMTK2 P30A, LMTK2 L780M, LMTK2 S910I, MATK A496T, MER E823Q, MER V8701, MET N375S, MET R988C, MET T1010I, MET V12381, NTRK1 H604Y, NTRK1 G613V, NTRK1 R780Q, NTRK2 D466fsX14, NTRK3 E402_F410delinsV, NTRK3 G466_Y529delinsD, NTRK3 R711_V712ins16, PDGFRA L221F, PDGFRA S478P, PDGFRB P345S, PDGFRB T464M, PTK-9 E195_V196insRPEDHIG, PYK2 G414V, PYK2 K838T, PYK2 V739_R780del, RET D489N, RET G691S, RET R982C, RON N440S, RON R523Q, RON Q473_D515del, RON R627fsX23, RON Y884_Q932del, RON R813_C814insQ, RON R1335G, ROR1 M518T, ROR2 T245A, ROR2 V819I, ROS T145P, ROS R167Q, ROS I537M, ROS S1109L, ROS D2213N, ROS K2228Q, ROS S2229C, ROS C76fsX, RYK N96S, RYK F516L, STYK G204S, TEK P346Q, TEK V486I, TEK V600L, TNK1 D472_R473del, TNK1 M598V, TNK1 M598fsX5, TXK R63C, TXK R336Q, TXK Y414fsX15, TYK2 V362F, TYK2 G363S, TYK2 I684S, TYK2 E971fsX67, TYRO3 I346N, VEGFR1 Y642H, VEGFR1 E982A, VEGFR1 P1201L, VEGFR2 V297I, VEGFR2 Q472H, VEGFR2 C482R, VEGFR2 P1147S, VEGFR3 Q890H, VEGFR3 R1321Q, ZAP70 K186fsX, and ZAP70 P296_S301del.

By “specific binding affinity” is meant that the antibody binds to the target kinase polypeptide with greater affinity than it binds to other polypeptides under specified conditions. Antibodies or antibody fragments are polypeptides that contain regions that can bind other polypeptides. The term “specific binding affinity” describes an antibody that binds to a mutant kinase polypeptide with significantly greater affinity than it binds to other polypeptides, e.g. the native kinase, under specified conditions.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods well known to those skilled in the art (see for example, Köhler et al., Nature 256:495-497 (1975), and U.S. Pat. No. 4,376,110, both of which are hereby incorporated by reference herein in their entirety including any figures, tables, or drawings).

The term “antibody fragment” refers to a portion of an antibody, often the hypervariable region and portions of the surrounding heavy and light chains that displays specific binding affinity for a particular molecule. A hypervariable region is a portion of an antibody that physically binds to the polypeptide target.

The term “domain” refers to a region of a polypeptide which contains a particular function. For instance, N-terminal or C-terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

An antibody of the invention may be isolated by comparing its binding affinity to a mutant kinase or kinase variant of the invention with its binding affinity to other polypeptides. Those which bind selectively to a mutant kinase or kinase variant of the invention would be chosen for use in methods requiring a distinction between a kinase of the invention and other polypeptides. Such methods could include, but should not be limited to, the analysis of altered kinase expression in tissue containing other polypeptides.

The mutant kinases and kinase variants of the present invention can be used in a variety of procedures and methods, such as for the generation of antibodies and for use in identifying pharmaceutical compositions. One skilled in the art will recognize that if an antibody is desired, a mutant kinase or kinase variant according to the invention could be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting.

In general, techniques for preparing monoclonal antibodies and hybridomas are well known in the art. Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art.

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horse radish peroxidase, alkaline phosphatase, and the like) fluorescent labels (such as FITC or rhodamine, and the like), paramagnetic atoms, and the like. Procedures for accomplishing such labeling are well-known in the art, for example, see Sternberger et al., J. Histochem. Cytochem. 18:315, 1970; Bayer et al., Meth. Enzym. 62:308-, 1979; Engval et al., Immunol. 109:129-, 1972; Goding, J. Immunol. Meth. 13:215-, 1976. The labeled antibodies of the present invention can be used for in vitro, in vivo, and in situ assays to identify cells or tissues which express a specific peptide.

The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art. The immobilized antibodies of the present invention can be used for in vitro, in vivo, and in situ assays as well as in immunochromatography.

The present invention also relates to a method of detecting a mutant kinase polypeptide or kinase variant in a sample, including: (a) contacting the sample with an above-described antibody, under conditions such that immunocomplexes form, and (b) detecting the presence of said antibody bound to the polypeptide. In detail, the methods include incubating a test sample with one or more of the antibodies of the present invention and assaying whether the antibody binds to the test sample. The presence of a mutant kinase or kinase variant of the invention in a sample may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluo-rescent assays) can readily be adapted to employ the antibodies of the present invention.

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed.

Methods for preparing protein extracts or membrane extracts of cells are well known in the art and can be readily be adapted in order to obtain a sample which is testable with the system utilized.

Diagnostic kits for performing such methods may contain all the necessary reagents to carry out the previously described methods of detection. The kit may include antibodies or antibody fragments specific for the mutant kinase or kinase variant as well as a conjugate of a binding partner of the antibodies or the antibodies themselves. Diagnostic kits for performing such methods may be constructed to include a first container containing the antibody and a second container having a conjugate of a binding partner of the antibody and a label, such as, for example, a radioisotope. In another embodiment, the kit further includes one or more other containers including one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

VI. Exemplary Protein Kinase Mutants and of the Invention and their Use

In one aspect the present invention relates to an isolated, enriched, or purified nucleic acid molecule that encodes a mutant of a protein kinase polypeptide with the protein kinase polypeptide being one of FGFR4, FGFR1, Tyro3, TEC, CSK and Ack1.

FGFR1 and FGFR4 are members of the fibroblast growth factor transmembrane receptor family. FGF-receptors stimulate growth of many cell types and are inter alia involved in tissue repair, wound healing and angiogenesis. The FGF receptors include a vatiety of splice variants. Each receptor and receptor splice variant is activated by a unique set of fibroblast growth factors (see Powers, C. M., et al., Endocr. Relat. Cancer 7:165-197 (2000), incorporated herein by reference in its entirety). Cellular signaling pathways by FGF receptors have recently been reviewed by Eswarakumar et al (Cytokine & Growth Factor Reviews 16, 139-149 (2005), incorporated herein by reference in its entirety). Fibroblast growth factor receptors are known to activate the Ras-MAPK, the PLCγ-PKC, the PI3K-Akt and the p38 MAPK pathways. They are also known to play a role in tumor development and progression. FGFR1 and FGFR4 are overexpressed in clinical prostate cancer and suppression of FGFR4 expression blocks prostate cancer proliferation (Sahadevan, K., et al. J. Pathology 213: 82-90 (2007), incorporated herein by reference in its entirety).

In one aspect the invention relates to a nucleic acid molecule that encodes FGFR4 Y367C. This nucleic acid accordingly encodes an FGFR protein that has at position 367 Cysteine rather than Tyrosine, as would be the case for the wild type protein. This amino acid is highly conserved throughout the FGFR family and located within the extracellular domain of the receptor. The amino acid exchange may facilitate receptor dimerization, thereby augmenting receptor activation and resulting in a basal receptor activation.

The present inventors have further identified an amino acid exchange at position 388 of FGFR4 as a single nucleotide polymorphism that is highly represented in Asian HCC patients compared to the Caucasian population. A respective protein has Arginine instead of Glycine at position 388. Furthermore, the homozygous genotype encoding Arginine at position 388 correlates with an increased secretion of a diagnostic marker for hepatocellular carcinoma.

In another aspect the invention also relates to a nucleic acid molecule that encodes FGFR1 P252S. Amino acid position 252 is highly conserved. The present inventors found a heterozygous exchange in the melanoma cell line MeWo in this position. This amino acid change may lead to receptor activation by influencing ligand binding. Without being bound by theory it is believed that the presence of a serine residue, providing a hydroxyl group, is likely to induce the formation of additional hydrogen bonds.

Tyro3, another receptor protein-tyrosine kinase, is a member of the Axl family, the members of which play an important role in spermatogenesis, immunoregulation, and phagocytosis. Tyro3 proteins are also known to be essential for mammalian development. The crystal structure of the N-terminal Ig domain pair of Tyro3 has been reported by Heiring et al. (J. Biol. Chem., 279, 8, 6952-6958, (2004)). In one aspect the invention relates to a nucleic acid molecule that encodes Tyro3 P822L. This nucleic acid accordingly encodes a Tyro 3 protein that has at position 822 Leucine rather than Proline, as would be the case for the wild type protein.

The present inventors observed that overexpression of Tyro3 in HEK293 cells confers resistance to apoptosis upon treatment with TNFα/actinomycin-D. Furthermore, overexpression of mutants S531L and P822L instead of wildtype Tyro3 enhanced anti-apoptotic effects. Tyro3 may perturb mitochondrial apoptotic signaling through the modulation of BCL2 family members. The present invention thus relates to the detection of Tyro3 expression, and particularly the occurrence of mutants S531L and P822L as genetic markers for chemoresistance. Inhibitors of Tyro3 signaling may be a promising adjuvant with other Chemotherapeutic agents.

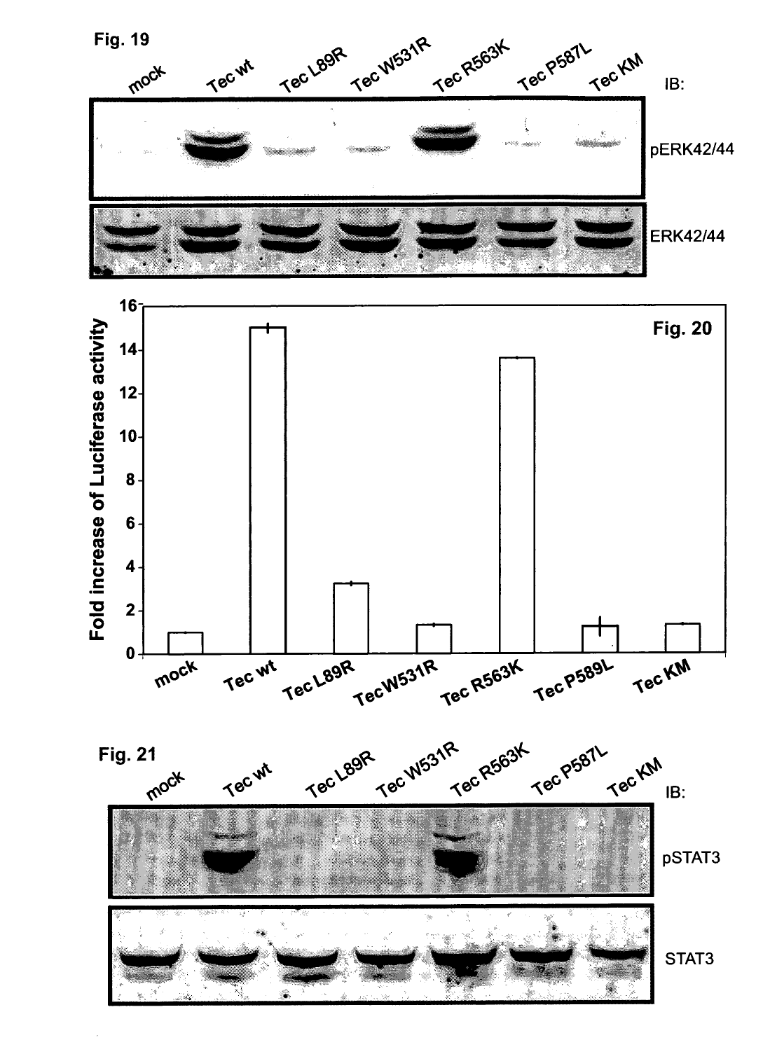

In a further aspect the invention also relates to a nucleic acid molecule that encodes TEC L89R, TEC W531R or TEC P587L. The first of these nucleic acids accordingly encodes a TEC protein that has at position 89 Arginine rather than Leucine, as would be the case for the wild type protein. This exchange is located in the pleckstrin homology domain of TEC. The second of these nucleic acids accordingly encodes a TEC protein that has at position 531 Arginine rather than Tryptophan, as would be the case for the wild type protein. This exchange is located in the kinase domain of TEC. The third of these nucleic acids encodes a TEC protein that has at position 587 Leucine rather than Proline, as would be the case for the wild type protein. This exchange is located in the kinase domain of TEC, too. The inventors have identified the corresponding proteins, encoded by these nucleic acids, as showing a decreased tyrosine phosphorylation compared to the wild type protein (see FIG. 17). Furthermore, the corresponding proteins are not able to activate MAPK signaling (FIG. 19), c-fos (FIG. 20) and Stat3 (FIG. 21).

TEC is a member of a family of intracellular tyrosine kinases of the same name, that includes Txk, Bmx, Itk, and Btk. TEC is involved in cell groth and differentiation and performs an essential role in antigen receptor signaling of T and B lymphocytes. It is important in phospholipase Cγ activation following antigen receptor stimulation. TEC is activated via phosphatidylinositol 3,4,5-trisphosphate generation by phosphatidylinositol 3-kinase (PI 3-kinase), and trans-phosphorylation by a Src family PTK, which activates the kinase domain of the protein. TEC is also involved in cytoskeleton reorganization by increasing actin polymerization and formation of stress fibers. The solution structure of the TEC Src homology 3 domain, which mediates interactions with proline-rich sequences (including in an intramolecular manner), was determined using NMR spectroscopy by Pursglove et al. (J. Biol. Chem., 277, 1, 755-762 (2002)). A tyrosine within this domain is phosphorylated during T cell signaling, a mechanism that depends on SH2-mediated interactions with the kinase domain (Joseph, R. E., Biochemistry, 46, 18, 5595-5603 (2007)). TEC is known to to signal constitutively when over-expressed in lymphocyte cell lines.

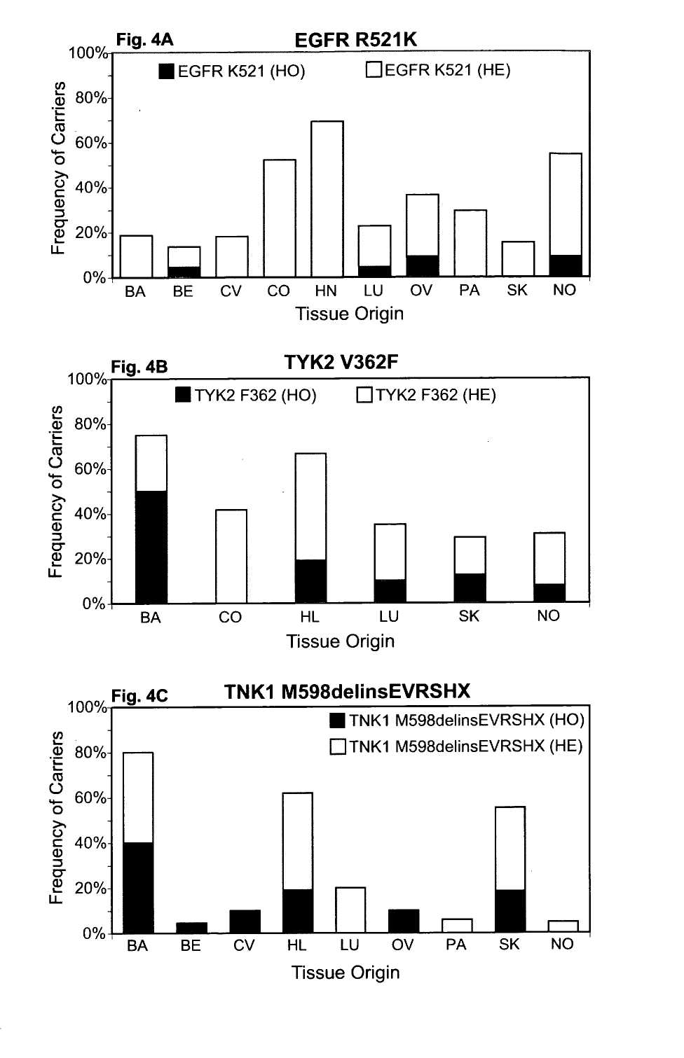

Tyrosine kinase 2 (Tyk2) is a Janus kinase transducing signals of cytokines. Tyk2 is known to play an important role in IFN-induced apoptosis of pro-B cells. Tyk2 is constitutively tyrosine phosphorylated in the leukemogenic cell line RH/K34 (Samaana, A., & Mahana, W., Immunology Letters 109, 2, 113-119 (2007)). Tyk2 has been shown to be play an important role in urokinase-type plasminogen activator-induced prostate cancer cell invasion Ode, H., et al., Biochem. Biophys. Res. Commun. doi:10.1016/j.bbrc.2007.08.160 (2007), incorporated herein by reference in its entirety). The present inventors identified a differential occurrence of TYK2 F362 allele carriers in tumor cell lines (FIG. 4B) with an under-representation of the TYK2 F362 allele in control samples. These data indicate a tumor-promoting function, including a cancer-promoting function, of TYK2 F362. Illustrative examples of a respective tumor include, but are not limited to, leukemia, melanoma, and glioma. The present invention thus also relates to the detection of TYK2 F362.

Ack1 is a nonreceptor tyrosine kinase that binds exclusively to activated Cdc42-GTP, a Rho family small G protein, but not to Rac or Rho (for an investigation on its biochemical properties see e.g. Yokoyama, N. et al., J. Biol. Chem., 278, 48, 47713-47723). Ack1 hass a kinase domain, an SH3 domain, a Cdc42/Rac-interactive binding (CRIB) domain, and a proline-rich C terminus. The C terminus has been shown to be involved in the interaction with the epidermal growth factor receptor (Shen, F., et al., Molecular Biology of the Cell, 18, 732-742 (2007), incorporated herein by reference in its entirety). Ack1 is Tyrosine phosphorylated by signaling via growth factors, cell adhesion, and muscarinic receptors. Ack1 has been shown to play an important role in cancer cell survival, as well as in tumor formation and metastasis (Mahajan, N. P., et al., Cancer Research 65, 10514-10523 (2005); van der Horst, E. H., Proc. Natl. Acad. Sci. U.S.A. 102, 44, 15901-15906 (2005)). MacKeigan et al. (Nat. Cell Biol. 7, 591-600 (2005)) identified Ack1 as an anti-apoptotic gene in an RNAi screen. Sustained activation of Ack-1 has been reported to be tumorigenic (Mahajan, 2005, supra; Mahajan, N. P., et al., Proc. Natl. Acad. Sci. U.S.A. 104, 20, 8438-8443 (2007), incorporated herein by reference in its entirety). Ack1 has also been shown to include an ubiquitin association domain at its C-terminus where it is ubiquitinylated and leading to its proteosomal degradation (Shen et al., 2007, supra). The present inventors show that the somatic variant of Ack1, that has at position 985 Serine rather than Asparagin, as would be the case for the wild type protein, is less sensitive to ubiquitination, suggesting the stabilization of this oncogenic kinase.

In this regard the invention also provides a method of identifying a cell having a predisposition to turn tumorigenic, including to transform into a cancer cell. The cell may in some embodiments be derived from an organism such as a mammal, a fish, an amphibian, or a bird. Examples of a mammal include, but are not limited to, a rat, a mouse, a rabbit, a Guinea pig, an opossum, a dog, a cat, a chimpanzee, a rhesus monkey, a cattle (cow), a marmoset and a human. The cell may for example be cultured. The cell may also be included in an organism such as a mammal (see above for examples), a fish, an amphibian, or a bird. In such embodiments the method may be or may be included in diagnosing the risk of developing a neoplasm in a subject. It may be or be included in diagnosis of a tumor such as cancer.

A respective method may include identifying the amino acid at position 367 of the expressed protein kinase FGFR4. The presence of Cysteine at position 367 indicates an increased predisposition to turn tumorigenic, including to transform into a cancer cell. A respective method may also include identifying the amino acid at position 388 of the expressed protein kinase FGFR4. The presence of Arginine at position 388 instead of Glycine indicates an increased predisposition to turn tumorigenic, including to transform into a cancer cell. In some embodiments such a cell is a liver cell. In some embodiments a method where the amino acid at position 388 of the expressed protein kinase FGFR4 is identified, the genotype of the gene encoding the FGFR4 receptor in the cell is further determined. A homozygous genotype FGFR4 388Arg, i.e. where the cell is homozygously encoding FGFR4 with Arginine at position 388, indicates an increased predisposition to transform into a cancer cell.

A respective method may also include identifying the amino acid at position 252 of the expressed protein kinase FGFR1. The presence of Serine at position 252 instead of Proline indicates an increased predisposition to turn tumorigenic, including to transform into a cancer cell. A respective method may also include identifying the amino acid at position 388 of the expressed protein kinase FGFR4. Such a cell is typically a hepatocyte. The presence of Arginine at position 388 instead of Glycine indicates an increased predisposition to transform into a hepatocellular carcinoma cell. A respective method may also include identifying the amino acid at position 531 and/or 822 of the expressed protein kinase Tyro3. The presence of Leucine at position 531 instead of Serine and/or the presence of Leucine at position 822 instead of Proline indicates an increased predisposition to turn tumorigenic, including to transform into a cancer cell. A respective method may also include identifying the amino acid at position 89 of the expressed protein kinase TEC. The presence of Arginine at position 89 instead of Leucine indicates an increased predisposition to turn tumorigenic, including to transform into a cancer cell. In some embodiments such a cell is a stomach cell. A respective method may also include identifying the amino acid at position 531 of the expressed protein kinase TEC. The presence of Arginine at position 531 instead of Tryptophan indicates an increased predisposition to turn tumorigenic, including to transform into a cancer cell. In some embodiments such a cell is a T cell. A respective method may also include identifying the amino acid at position 587 of the expressed protein kinase TEC. The presence of Leucine at position 587 instead of Proline indicates an increased predisposition to turn tumorigenic, including to transform into a cancer cell. In some embodiments such a cell is a lung cell. Such a method may also include identifying the amino acid at position 362 of the expressed protein kinase TYK2. The presence of Phenylalanine at position 362 instead of Valine indicates an increased predisposition to turn tumorigenic, including to transform into a cancer cell. In some embodiments such a cell is a brain cell or a cell of the hematopoietic/lymphoid system. A respective method may also include identifying the amino acid at position 26 of the expressed protein kinase C-terminal Src kinase (CSK). The presence of an amino acid different from Glutamine at position 26 indicates an increased predisposition to turn tumorigenic, including to transform into a cancer cell. In some embodiments such a cell is a colon cell. A respective method may also include identifying the amino acid at position 985 of the expressed protein kinase Ack1. The presence of Asparagine at position 985 instead of Serine indicates an increased predisposition to turn tumorigenic, including to transform into a cancer cell. In some embodiments such a cell is a kidney cell.

A method of identifying a cell that has a predisposition to turn tumorigenic, including to transform into a cancer cell, may be used in combination with any other diagnostic or prognostic method, e.g. a method of cancer prognosis or diagnosis. As an illustrative example, where the cell of interest is a liver cell, the level of the marker protein alpha-fetoprotein may be determined (see above and below). The method of the invention may also be combined with any other desired method. In some embodiments a method according to the present invention may be combined with detecting the expression of one or more marker genes of the respective tissue type of the cell in question. Any such combination may also be carried out with a respective method of identifying a cell that is resistant to apoptosis inducing reagents (see below).

In this regard the invention further provides a method of identifying a cell that is resistant to apoptosis inducing reagents, i.e. a cell that is chemoresistant. A respective method may include measuring in the cell the expression of the protein kinase Tyro3. Such a method further includes comparing the result of the measurement obtained with the result of a control measurement. An increased expression of protein kinase Tyro3 indicates resistance of the cell to apoptosis inducing reagents. A respective method may include identifying the amino acid at position 531 of the expressed protein kinase Tyro3. The presence of Leucine at position 531 instead of Serine indicates increased resistance of the cell to apoptosis inducing reagents. A respective method may include identifying the amino acid at position at position 822 of the expressed protein kinase Tyro3. The presence of Leucine at position 822 instead of Proline indicates increased resistance of the cell to apoptosis inducing reagents. Furthermore, a respective method may include identifying the amino acid at position 89 of the expressed protein kinase TEC. The presence of Arginine at position 89 instead of Leucine indicates increased resistance of the cell to apoptosis inducing reagents. In some embodiments the respective cell is a T cell. A respective method may also include identifying the amino acid at position 531 of the expressed protein kinase TEC. The presence of Arginine at position 531 instead of Tryptophan indicates increased resistance of the cell to apoptosis inducing reagents. In some embodiments the respective cell is a T cell. A respective method may include identifying the amino acid at position 587 of the expressed protein kinase TEC. The presence of Leucine at position 587 instead of Proline indicates increased resistance of the cell to apoptosis inducing reagents. In some embodiments the respective cell is a T cell. One of the amino acid exchanges as described above may for instance confer chemoresistance through enhancing antiapoptotic effects in a cell, including a cancer cell. It is understood that any of the methods described above may be combined.

### VII. Identification of Compounds Modulating Mutant Kinase Activity

Encompassed by the instant disclosure are also methods for the identification of a compound capable of modulating the activity of a mutant protein kinase polypeptide or protein kinase polypeptide variant of the invention. Said mutant kinase polypeptide or protein kinase variant is selected from the ones detailed above.

The term “kinase activity”, as used herein, may relate to the catalytic activity of a kinase and thus define the rate at which a kinase catalytic domain phosphorylates a substrate. Catalytic activity can be measured, for example, by determining the amount of a substrate converted to a phosphorylated product as a function of time. Catalytic activity can be measured by methods of the invention by holding time constant and determining the concentration of a phosphorylated substrate after a fixed period of time. Phosphorylation of a substrate occurs at the active site of a protein kinase. The active site is normally a cavity in which the substrate binds to the protein kinase and is phosphorylated. The term “kinase activity” may also relate to the binding of a kinase to a natural binding partner that may, but must not include phosphorylation.

The term “kinase catalytic domain” refers to a region of the protein kinase that is typically 25-300 amino acids long and is responsible for carrying out the phosphate transfer reaction from a high-energy phosphate donor molecule such as ATP or GTP to itself (autophosphorylation) or to other proteins (heterologous phosphorylation). The catalytic domain of protein kinases is made up of 12 subdomains that contain highly conserved amino acid residues, and are responsible for proper polypeptide folding and for catalysis. The catalytic domain can be identified following, for example, a Smith-Waterman alignment of the protein sequence against the non-redundant protein database.

The term “substrate” as used herein refers to a molecule phosphorylated by a kinase of the invention. Kinases phosphorylate substrates on serine/threonine or tyrosine amino acids. The molecule may be another protein or a polypeptide.

By “functional” domain is meant any region of the polypeptide that may play a regulatory or catalytic role as predicted from amino acid sequence homology to other proteins or by the presence of amino acid sequences that may give rise to specific structural conformations (i.e. coiled-coils).

The term “modulates” refers to the ability of a compound to alter the function of a mutated kinase or kinase variant of the invention. A modulator typically activates or inhibits the activity of a mutated kinase or kinase variant of the invention depending on the concentration of the compound exposed to the kinase. In some embodiments the modulator inhibits the activity of a mutated kinase or kinase variant of the invention. The compound may be capable of differentiating between a native and mutant form and/or between the distinct variants of said kinase.

The term “activates” refers to increasing the cellular activity of the kinase. The term “inhibits” refers to decreasing the cellular activity of the kinase. Kinase activity may be the interaction with a natural binding partner, including phosphorylation.

The term “modulates” also refers to altering the function of mutant kinases of the invention by increasing or decreasing the probability that a complex forms between the kinase and a natural binding partner. A modulator may increase or decrease the probability that such a complex forms between the kinase and the natural binding partner depending on the concentration of the compound exposed to the kinase. In some embodiments the modulator decreases the probability that a complex forms between the kinase and the natural binding partner.

The term “complex” refers to an assembly of at least two molecules bound to one another. Signal transduction complexes often contain at least two protein molecules bound to one another. For instance, a protein tyrosine receptor protein kinase, GRB2, SOS, RAF, and RAS assemble to form a signal transduction complex in response to a mitogenic ligand.

The term “natural binding partner” refers to polypeptides, lipids, small molecules, or nucleic acids that bind to kinases in cells. The natural binding partner may be a nucleotide, such as ATP or GTP or an analogue thereof, or a protein. In some embodiments it is a protein that is involved in signal transduction pathways. A change in the interaction between a kinase and a natural binding partner can manifest itself as an increased or decreased probability that the interaction forms, or an increased or decreased concentration of kinase/natural binding partner complex.

Such a method includes the steps of: (a) contacting a mutant kinase polypeptide or kinase variant of the invention with a test substance; (b) measuring the activity of said polypeptide; and (c) determining whether said substance modulates the activity of said polypeptide.

Also within the scope of the invention are methods for the identification of mutant kinase polypeptide or kinase variant modulating compounds in a cell. Said method includes (a) expressing a mutant kinase polypeptide or kinase variant in a cell; (b) adding a test substance to said cell; and (c) monitoring a change in cell phenotype or the interaction between said polypeptide and a natural binding partner.

In one embodiment, the cells used for such a method are cancer cells, for instance a cancer cell line. Examples for cells that may be suitable for such a method are those identified in the Examples section of the present invention, i.e. the cells in which the mutant kinases or kinase variants were first identified.

The term “expressing” as used herein refers to the production of mutant kinases or kinase variants of the invention from a nucleic acid vector containing kinase genes within a cell. The nucleic acid vector is transfected into cells using well-known techniques in the art as described herein.

The invented methods thus also relate to the detection of an agonist or antagonist of mutant kinase or kinase variant activity including incubating cells that produce a mutant kinase of the invention in the presence of a compound and detecting changes in the level of kinase activity. The compounds thus identified would produce a change in activity indicative of the presence of the compound. The compound may be present within a complex mixture, for example, serum, body fluid, or cell extracts. Once the compound is identified it can be isolated using techniques well known in the art.

The present invention also encompasses a method of agonizing (stimulating) or antagonizing kinase associated activity in a cell and/or in an organism such as a mammal (see below for examples) including administering to said cell and/or organism an agonist or antagonist to a kinase of the invention in an amount sufficient to effect said agonism or antagonism. As an illustrative example a protein kinase inhibitor (see also below) or protein kinase acrivator in form of a synthetic small organic compound may be used for this purpose. Recent overviews on protein kinase inhibitors have for instance been given by Dancey & Sausville (Nature Reviews Drug Discovery 2, 4, 296-313 (2007)), Thaimattam et al. (Current Pharmaceutical Design 13, 2751-2765 (2007)) and Liao (J. Med. Chem. 50, 3, 409-424 (2007)).

In some embodiments agonizing (stimulating) or antagonizing kinase associated activity in a mammal includes stimulating or reducing the expression or amplification of the respective protein kinase. Methods of stimulating or reducing the expression or amplification of a protein are well known in the art. As an illustrative example, agonizing kinase associated activity may in some embodiments be achieved by expression of a corresponding heterologous kinase. Tissue selective expression of such a heterologous kinase, for example expression only in the liver, may be achieved by using the microRNA present in the respective cell or organism in controlling expression, as described by Brown et al. (Nature Biotechnology doi:10.1038/nbt1372 (2007)).

As a further illustrative example, in some embodiments of the present method of the invention the expression of the respective protein kinase is reduced by means of a non-coding nucleic acid molecule, such as for example an aptamer or a Spiegelmer® (described in WO 01/92655). A non-coding nucleic acid molecule may also be an nc-RNA molecule (see e.g. Costa, F F, Gene (2005), 357, 83-94 for an introduction on natural nc-RNA molecules). Examples of nc-RNA molecules include, but are not limited to, an anti-sense-RNA molecule, an L-RNA Spiegelmer®, a silencer-RNA molecule (such as the double-stranded Neuron Restrictive Silencer Element), a micro RNA (miRNA) molecule, a short hairpin RNA (shRNA) molecule, a small interfering RNA (siRNA) molecule, a repeat-associated small interfering RNA (rasiRNA) molecule or an RNA that interacts with Piwi proteins (piRNA) (for a brief review see e.g. Lin, H., Science (2007) 316, 397).

The use of small interfering RNAs has become a tool to “knock down” specific genes. An overview on the differences between the use of synthetic small organic compounds and RNAi has been given by Weiss et al. (Nature Chem. Biol. 3, 12, 739-744 (2007)). Small interfering RNA makes use of gene silencing or gene suppression through RNA interference (RNAi), which occurs at the posttranscriptional level and involves mRNA degradation. RNA interference represents a cellular mechanism that protects the genome. SiRNA molecules mediate the degradation of their complementary RNA by association of the siRNA with a multiple enzyme complex to form what is called the RNA-induced silencing Complex (RISC). The siRNA becomes part of RISC and is targeted to the complementary RNA species which is then cleaved. This leads to the loss of expression of the respective gene (for a brief overview see Zamore, P D, & Haley, B, Science 309, 1519-1524 [2005]). This technique has for example been applied to silencing parasitic DNA sequences, such as the cleavage of HIV RNA, as disclosed in US patent application 2005/0191618.

A typical embodiment of such a siRNA for the current invention includes an in vitro or in vivo synthesized molecule of 10 to 35 nucleotides, in some embodiments 15 to 25 nucleotides. A respective si-RNA molecule may be directly synthesized within a cell of interest (including a cell that is part of a microorganism and an animal). It may also be introduced into a respective cell and/or delivered thereto. An illustrative example of delivering a siRNA molecule into selected cells in vivo is its non-covalent binding to a fusion protein of a heavy-chain antibody fragment (Fab) and the nucleic acid binding protein protamin (Song, E. et al., Nature Biotech. 23, 6, 709-717 [2005]). In an embodiment of the present invention siRNA molecules are used to induce a degradation of mRNA molecules encoding one or more protein kinases of interest.

A method of treating diseases in a mammal with a modulator of mutant kinase or kinase variant activity including administering the compound to a mammal in an amount sufficient to modulate mutant kinase or kinase variant associated functions is also encompassed in the present application.

In an effort to discover novel treatments for diseases, biomedical researchers and chemists have designed, synthesized, and tested molecules that inhibit the function of protein kinases. Some small organic molecules form a class of compounds that modulate the function of protein kinases. Examples of molecules that have been reported to inhibit the function of protein kinases include, but are not limited to, bis monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al).

Compounds that can traverse cell membranes and are resistant to acid hydrolysis are potentially advantageous as therapeutics as they can become highly bioavailable after being administered orally to patients. However, many of these protein kinase inhibitors only weakly inhibit the function of protein kinases. In addition, many inhibit a variety of protein kinases and will cause multiple side-effects as therapeutics for diseases.

Other examples of substances capable of modulating kinase activity include, but are not limited to, indolinones, tyrphostins, quinazolines, quinoxolines, and quinolines. The indolinones, quinazolines, tyrphostins, quinolines, and quinoxolines referred to above include well known compounds such as those described in the literature.

For example, representative publications describing indolinone compounds include WO 96/22976 (published Aug. 1, 1996 by Ballinari et al.), U.S. patent application Ser. Nos. 08/702,232 and 08/485,323 by Tang et al., and International Patent Publication WO 96/22976 by Ballinari et al.; all of which are incorporated herein by reference in their entirety, including any drawings.

Publications relating to the use of quinazolines as kinase function modulators include Barker et al., EPO Publication No. 0 520 722 A1; Jones et al., U.S. Pat. No. 4,447,608; Kabbe et al., U.S. Pat. No. 4,757,072; Kaul and Vougioukas, U.S. Pat. No. 5,316,553; Kreighbaum and Corner, U.S. Pat. No. 4,343,940; Pegg and Wardleworth, EPO Publication No. 0 562 734 A1; Barker et al., Proc. of Am. Assoc. for Cancer Research 32:327 (1991); Bertino, J. R., Cancer Research 3:293-304 (1979); Bertino, J. R., Cancer Research 9(2 part 1):293-304 (1979); Curtin et al., Br. J. Cancer 53:361-368 (1986); Fernandes et al., Cancer Research 43:1117-1123 (1983); Ferris et al. J. Org. Chem. 44(2):173-178; Fry et al., Science 265:1093-1095 (1994); Jackman et al., Cancer Research 51:5579-5586 (1981); Jones et al. J. Med. Chem. 29(6):1114-1118; Lee and Skibo, Biochemistry 26(23):7355-7362 (1987); Lemus et al., J. Org. Chem. 54:3511-3518 (1989); Ley and Seng, Synthesis 1975:415-522 (1975); Maxwell et al., Magnetic Resonance in Medicine 17:189-196 (1991); Mini et al., Cancer Research 45:325-330 (1985); Phillips and Castle, J. Heterocyclic Chem. 17(19):1489-1596 (1980); Reece et al., Cancer Research 47(11):2996-2999 (1977); Sculier et al., Cancer Immunol. and Immunother. 23:A65 (1986); Sikora et al., Cancer Letters 23:289-295 (1984); Sikora et al., Analytical Biochem. 172:344-355 (1988); all of which are incorporated herein by reference in their entirety, including any drawings.

Quinoxalines are, for example, described in Kaul and Vougioukas, U.S. Pat. No. 5,316,553, incorporated herein by reference in its entirety, including any drawings.

Quinolines are described in Dolle et al., J. Med. Chem. 37:2627-2629 (1994); MaGuire, J. Med. Chem. 37:2129-2131 (1994); Burke et al., J. Med. Chem. 36:425-432 (1993); and Burke et al. BioOrganic Med. Chem. Letters 2:1771-1774 (1992), all of which are incorporated by reference in their entirety, including any drawings.

Tyrphostins are described in Allen et al., Clin. Exp. Immunol. 91:141-156 (1993); Anafi et al., Blood 82:12:3524-3529 (1993); Baker et al., J. Cell Sci. 102:543-555 (1992); Bilder et al., Amer. Physiol. Soc. pp. 6363-6143:C721-C730 (1991); Brunton et al., Proceedings of Amer. Assoc. Cancer Rsch. 33:558 (1992); Bryckaert et al., Experimental Cell Research 199:255-261 (1992); Dong et al., J. Leukocyte Biology 53:53-60 (1993); Dong et al., J. Immunol. 151(5):2717-2724 (1993); Gazit et al., J. Med. Chem. 32:2344-2352 (1989); Gazit et al., J. Med. Chem. 36:3556-3564 (1993); Kaur et al., Anti-Cancer Drugs 5:213-222 (1994); Kaur et al., King et al., Biochem. J. 275:413-418 (1991); Kuo et al., Cancer Letters 74:197-202 (1993); Levitzki, A., The FASEB J. 6:3275-3282 (1992); Lyall et al., J. Biol. Chem. 264:14503-14509 (1989); Peterson et al., The Prostate 22:335-345 (1993); Pillemer et al., Int. J. Cancer 50:80-85 (1992); Posner et al., Molecular Pharmacology 45:673-683 (1993); Rendu et al., Biol. Pharmacology 44(5):881-888 (1992); Sauro and Thomas, Life Sciences 53:371-376 (1993); Sauro and Thomas, J. Pharm. and Experimental Therapeutics 267(3):119-1125 (1993); Wolbring et al., J. Biol. Chem. 269(36):22470-22472 (1994); and Yoneda et al., Cancer Research 51:4430-4435 (1991); all of which are incorporated herein by reference in their entirety, including any drawings.

Other compounds that could be used as modulators include oxindolinones such as those described in U.S. patent application Ser. No. 08/702,232 filed Aug. 23, 1996, incorporated herein by reference in its entirety, including any drawings.

Other substances that modulate the activity of the mutant kinases may include antisense oligonucleotides and antibodies.

### VIII. Methods of Use of the Molecules of the Invention

The present invention also includes a method for screening for human cells containing a mutant kinase polypeptide or kinase polypeptide variant of the invention or an equivalent sequence. The method involves identifying the mutant kinase polypeptide or kinase variant in human cells using techniques that are routine and standard in the art (e.g., cloning, Southern or Northern blot analysis, in situ hybridization, PCR amplification, etc.).

Also provided are methods for treating or preventing a disease or disorder by administering to a patient in need of such treatment a substance that modulates the activity of a mutant kinase or kinase variant of the invention. Methods of identifying such compounds have been discussed above. In some embodiments the disease or disorder to be treated or prevented involves an aberrant signal transduction pathway, for example an aberrant kinase function due to a mutation or germline alteration. The disease or disorder to be treated or prevented with the methods of the invention may for example be cancer.

If the aberrant kinase function is due to a mutation, the mutation can be an activating mutation, i.e. a mutation that leads to the constitutive activation of the kinase. Such a mutation may for example impair the intermolecular or intramolecular regulation of the kinase.

Alternatively, a germline alteration in a kinase gene, as disclosed and discussed above, may also lead to an altered function of a kinase. This altered function may include deregulation of the kinase, enhanced activity, and increased or decreased sensitivity against natural binding partners or drugs, such as known kinase modulating compounds. Such changes of the function of the kinase by germline alterations may inter alia lead to a predisposition for the development of a proliferative disease, such as cancer, as tumor development and progression naturally depend on an accumulation of a number of aberrations, of which alteration of kinase function may be only one aspect.

The term “preventing” refers to decreasing the probability that an organism contracts or develops an abnormal condition.

The term “treating” refers to having a therapeutic effect and at least partially alleviating or abrogating an abnormal condition in the organism.

The term “administering” relates to a method of incorporating a compound into cells or tissues of an organism.

The term “signal transduction pathway” refers to the molecules that propagate an extracellular signal through the cell membrane to become an intracellular signal. This signal can then stimulate a cellular response. The polypeptide molecules involved in signal transduction processes are typically protein kinases, more specifically receptor and non-receptor protein tyrosine kinases, serine/threonine kinases and dual specificity kinases. Also involved are typically receptor and non-receptor protein phosphatases, nucleotide exchange factors, and transcription factors. Signal transduction may be mediated via a variety of signaling domains, including but not limited to SRC homology 2 and 3 domains (SH2 and SH3), phosphotyrosine binding domains (PTB), pleckstrin homology domains (PH), proline-rich regions, coiled-coil structures, WW domains, etc., all of which are known to the person skilled in the art.

The term “therapeutic effect” refers to the inhibition or activation of factors causing or contributing to the abnormal condition. A therapeutic effect relieves to some extent one or more of the symptoms of the abnormal condition.

The term “aberration” or “aberrant”, in conjunction with the function of a kinase in a signal transduction process, refers to a kinase that is over- or under-expressed in an organism, altered such that its catalytic activity is lower or higher than wild-type protein kinase activity, altered such that it can no longer interact with a natural binding partner, is no longer modified by another protein kinase or protein phosphatase, or no longer interacts with a natural binding partner.

The abnormal condition caused by a mutant kinase polypeptide or kinase variant of the invention may be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then be monitored. The organism may or instance be a mammal, such as a mouse, a rat, a rabbit, a guinea pig, a goat, a monkey or an ape. In some embodiments the organism is a human.

The term “abnormal condition” refers to a function in the cells or tissues of an organism that deviates from their normal functions in that organism. An abnormal condition can relate to cell proliferation, cell differentiation, or cell survival.

Abnormal cell proliferative conditions include cancer, fibrotic and mesangial disorders, abnormal angiogenesis and vasculogenesis, wound healing, psoriasis, diabetes mellitus, and inflammation. Furthermore, said proliferative disorders can relate to conditions in which programmed cell death (apoptosis) pathways are abrogated. As a number of protein kinases are associated with the apoptosis pathways, aberrations in the function of any one of the protein kinases could lead to cell immortality.

Other methods included in the invention are useful for the detection of a mutant kinase polypeptide or kinase variant in a sample as a diagnostic tool for diseases or disorders.

Such a method may include the steps of: (a) contacting the sample with a nucleic acid probe which hybridizes under hybridization assay conditions to a target region of a nucleic acid encoding a mutant kinase polypeptide or kinase variant of the invention or the complement thereof; and (b) detecting the presence or amount of the probe:target region hybrid as an indication of the disease or disorder.

The disease or disorder involving a kinase mutant or kinase variant may be a proliferative disease or disorder, for example cancer.

In some embodiments the nucleic acid probe hybridizes to a mutant kinase target region that encodes at least about 10, about 15, about 20, about 30, about 40, about 50, about 75, about 100, about 150, about 200, about 250, about 300 or about 350 contiguous amino acids of the sequence set forth in SEQ ID NO: 1-256 or 513-642, or the corresponding full-length amino acid sequence, or a functional derivative thereof, with the proviso that the target region includes one of the mutations or alterations set forth in FIGS. 32 and 33. Hybridization conditions should be such that hybridization occurs only with the kinase genes in the presence of other nucleic acid molecules. Under stringent hybridization conditions only highly complementary nucleic acid sequences hybridize. It may be desired to use conditions that prevent hybridization of nucleic acids that have one or more mismatches in a sequence of about 20 contiguous nucleotides.

The diseases that may be diagnosed by detection of a kinase nucleic acid in a sample may include a cancer. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well-known in the art and can be readily adapted in order to obtain a sample that is compatible with the method utilized.

### IX. Pharmaceutical Formulations and Routes of Administration

The compounds described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences, Mack Publishing Co., Easton, Pa., latest edition”. Exemplary routes include, but are not limited to, oral, transdermal, and parenteral delivery.

Suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Pharmaceutical compositions that include the compounds of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the active compounds into preparations that can be used pharmaceutically. Proper formulation is dependent upon the route of administration chosen.

For injection, the agents of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system including benzyl alcohol, a non-polar surfactant, a water-miscible organic polymer, and an aqueous phase. The co-solvent system may be the VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: D5W) consists of VPD diluted 1:1 with a 5% dextrose in water solution.

This co-solvent system dissolves hydrophobic compounds well, and itself produces low toxicity upon systemic administration. Naturally, the proportions of a co-solvent system may be varied considerably without destroying its solubility and toxicity characteristics.

Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may include suitable solid or gel phase carriers or excipients.

Examples of such carriers or excipients include but are not limited to calcium carbonate, calcium phosphate, various sugars, starches, cellulose derivatives, gelatin, and polymers such as polyethylene glycols.

Many of the kinase modulating compounds of the invention may be provided as salts with pharmaceutically compatible counter-ions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the kinase activity). Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. It may be desired to use compounds that exhibit high therapeutic indices. The data obtained from these cell culture assays and animal studies can be used in formulating a range of dosage for use in humans. The dosage of such compounds lies preferably within a range of circulating concentrations that include the EDₛₒ with little or no toxicity. The dosage may vary within this range depending upon the dosage form employed and the route of administration utilized. The exact formulation, route of administration and dosage can be chosen by the individual physician in view of the patient's condition. (See e.g., Fingl, et al. (1975) The Pharmacological Basis of Therapeutics Chapter 1 page 1).

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, for example from about 30 to about 90%, such as from about 50 to about 90%.

In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labeling approved by the U.S. Food and Drug Administration or other government agency for prescription drugs, or the approved product insert.

Compositions including a compound of the invention formulated in a compatible pharmaceutical carrier may also be prepared, placed in an appropriate container, and labeled for treatment of an indicated condition. Suitable conditions indicated on the label may include, for example, treatment of cancer.

In order that the invention may be readily understood and put into practical effect, particular embodiments will now be described by way of the following non-limiting examples.

### Exemplary Embodiments of the Invention

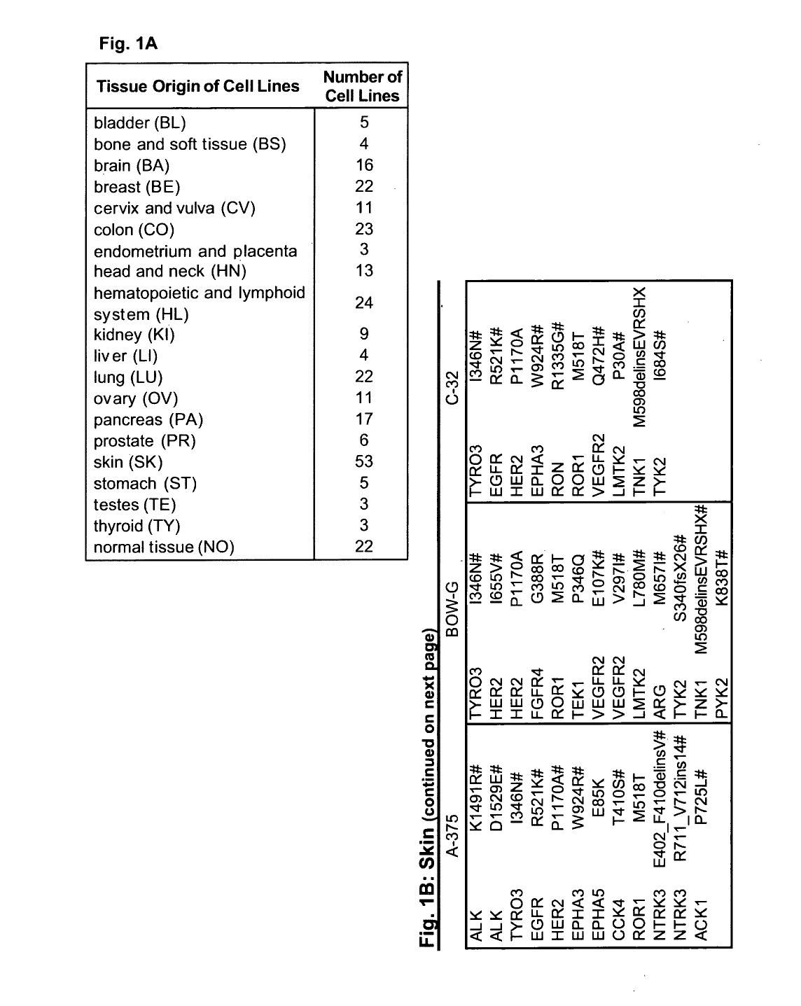

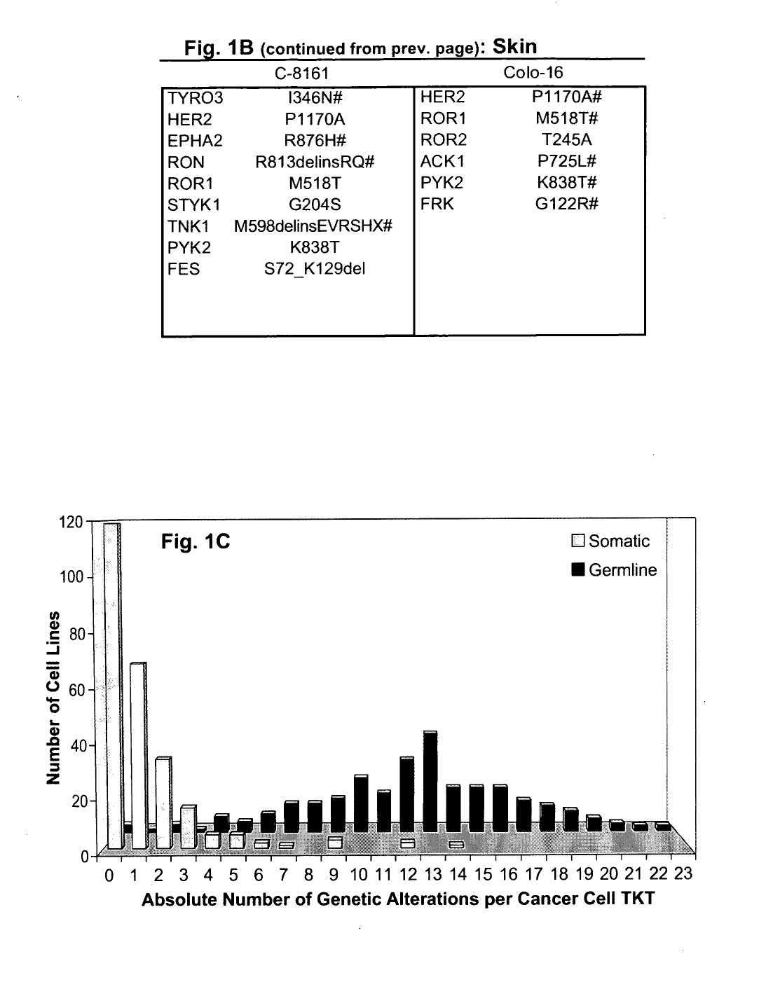

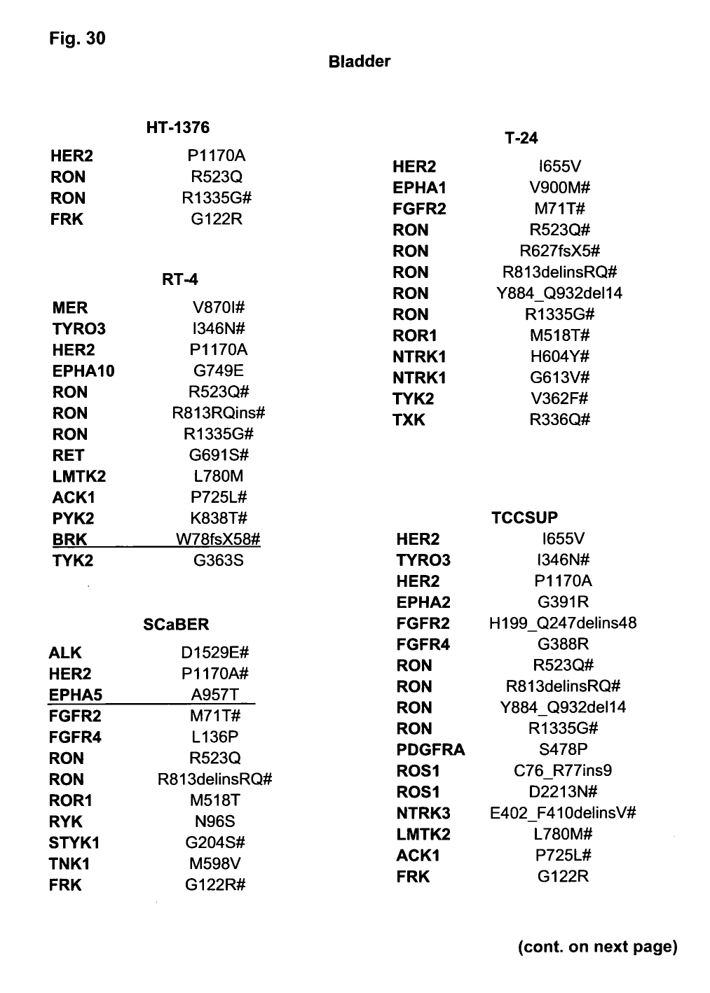

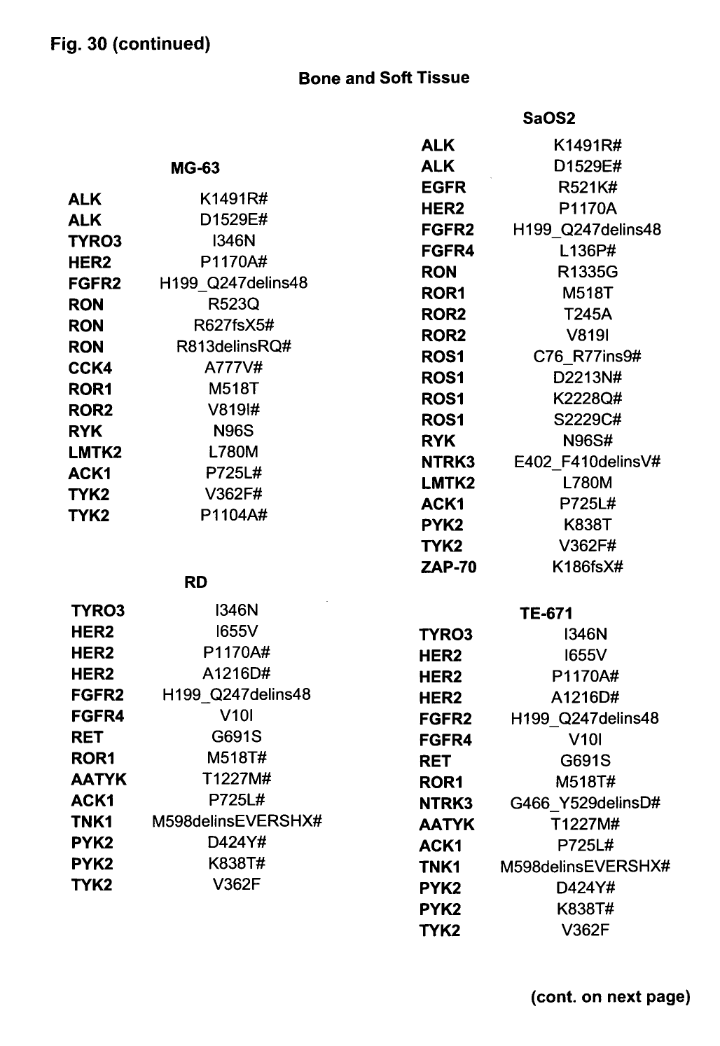

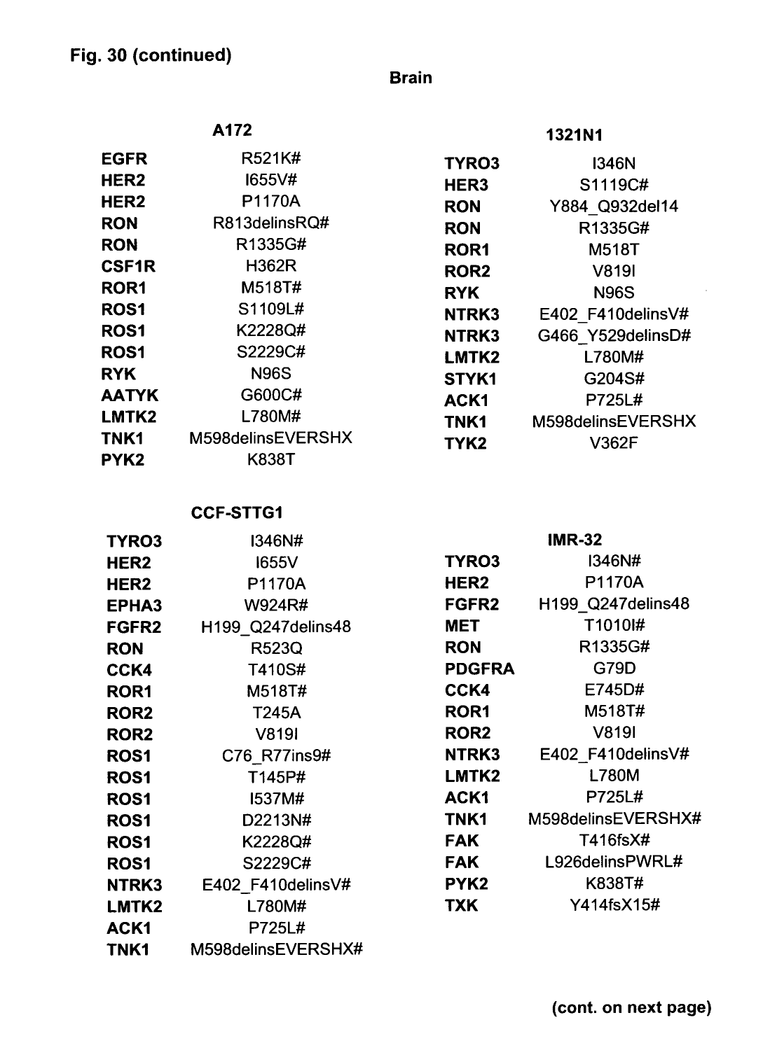

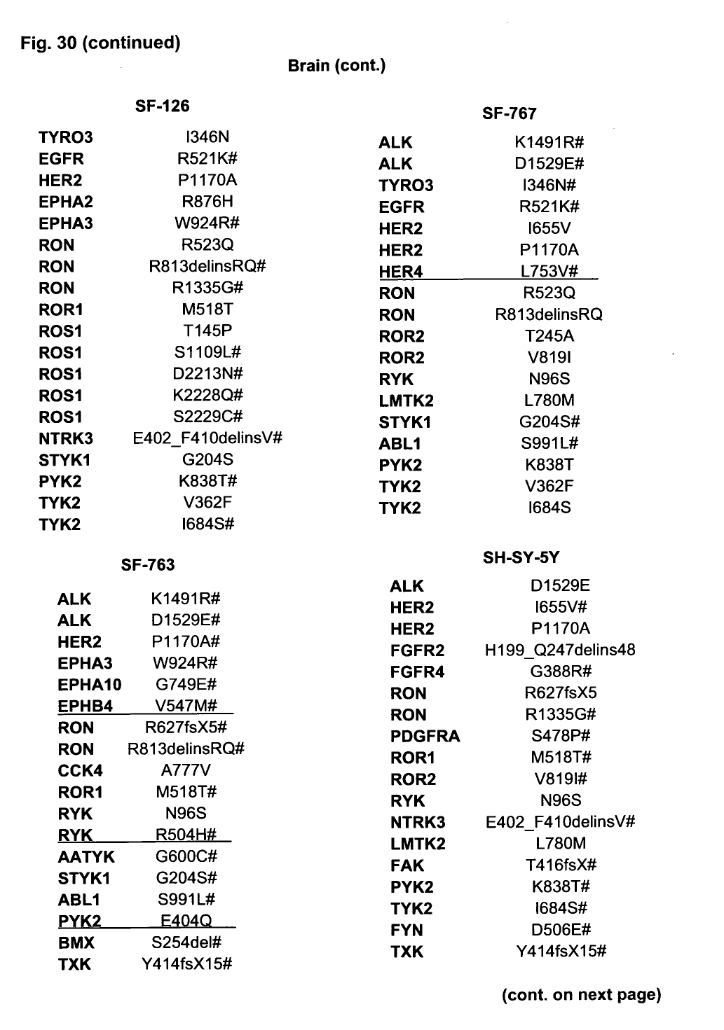

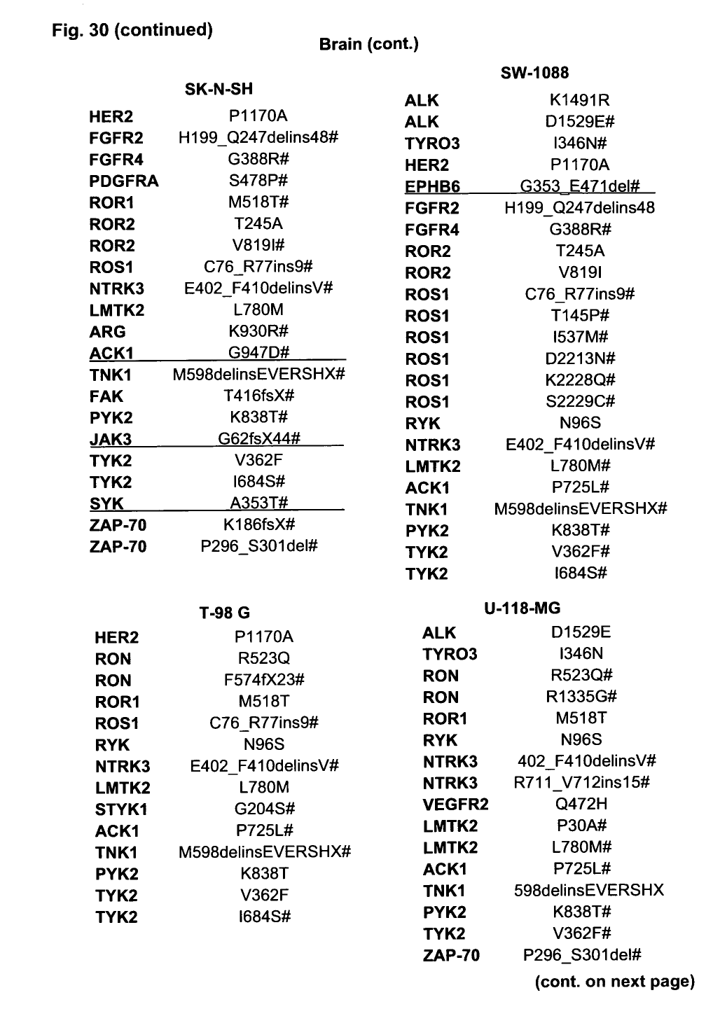

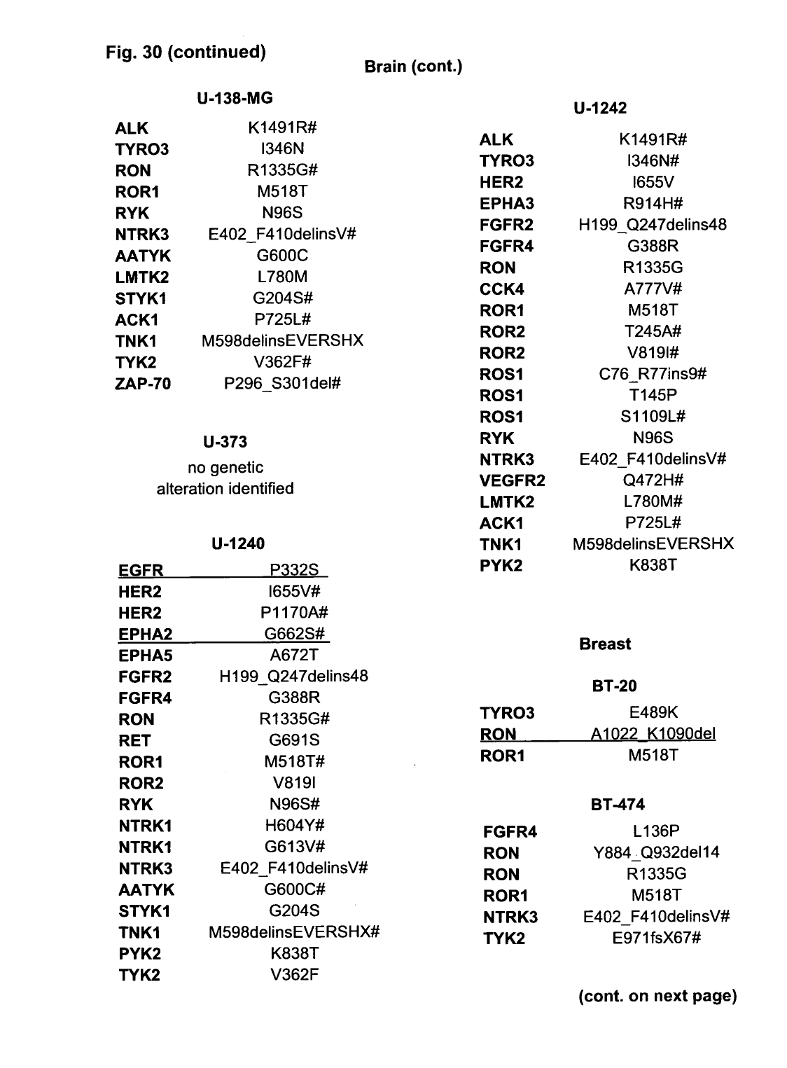

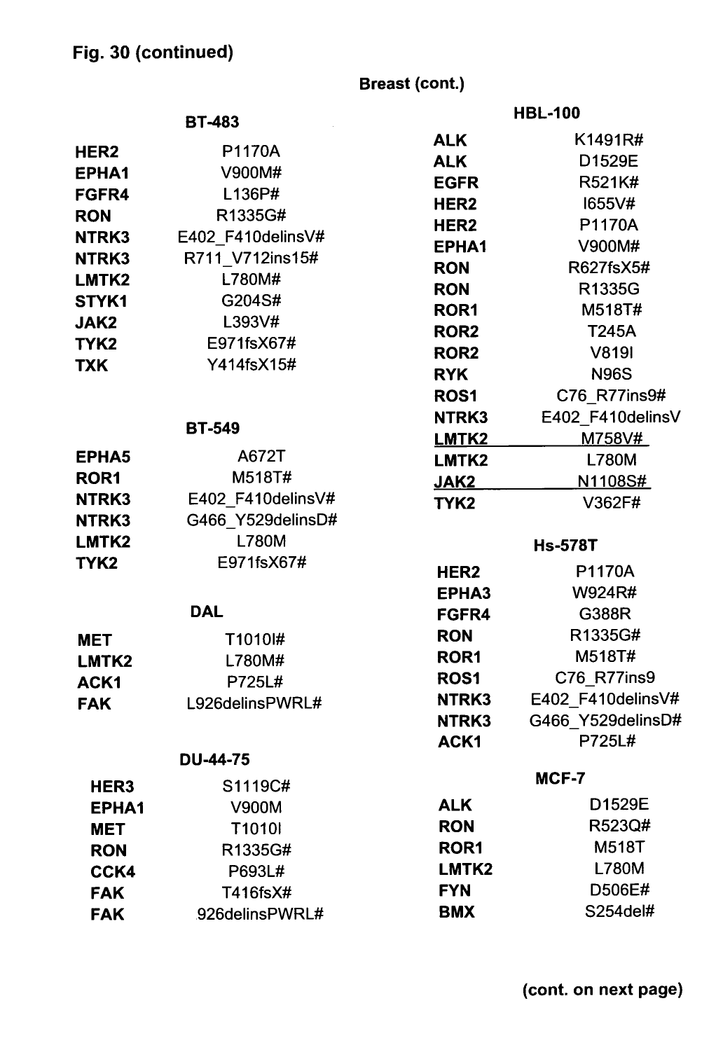

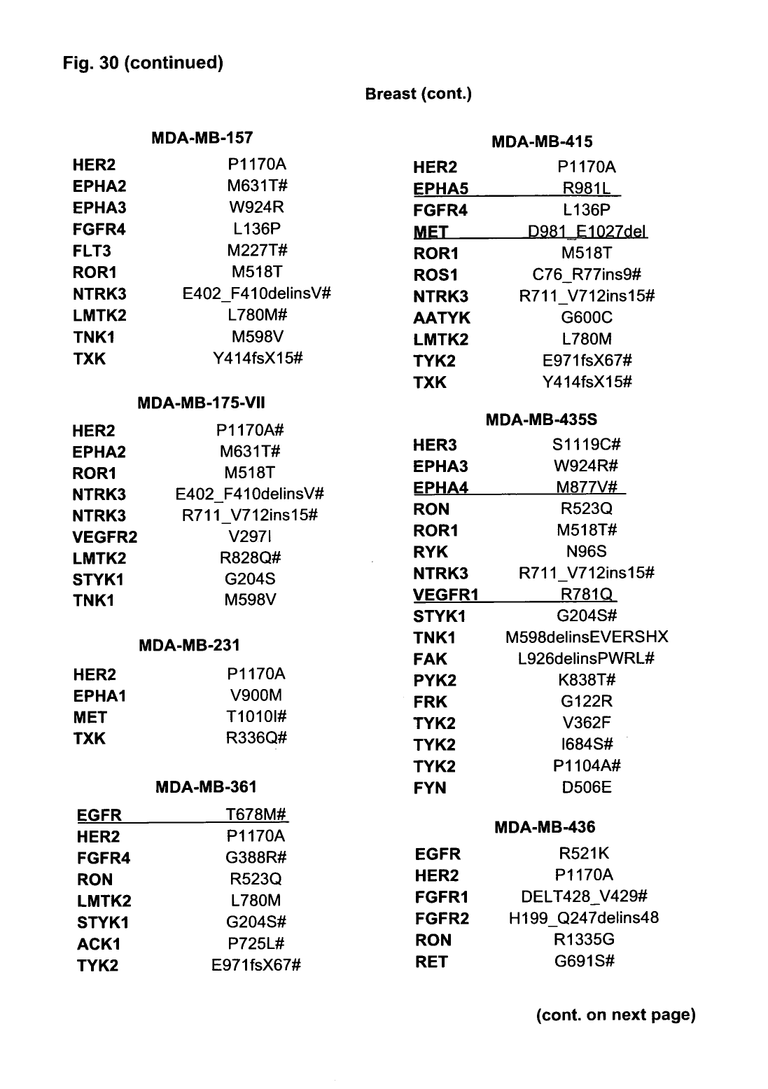

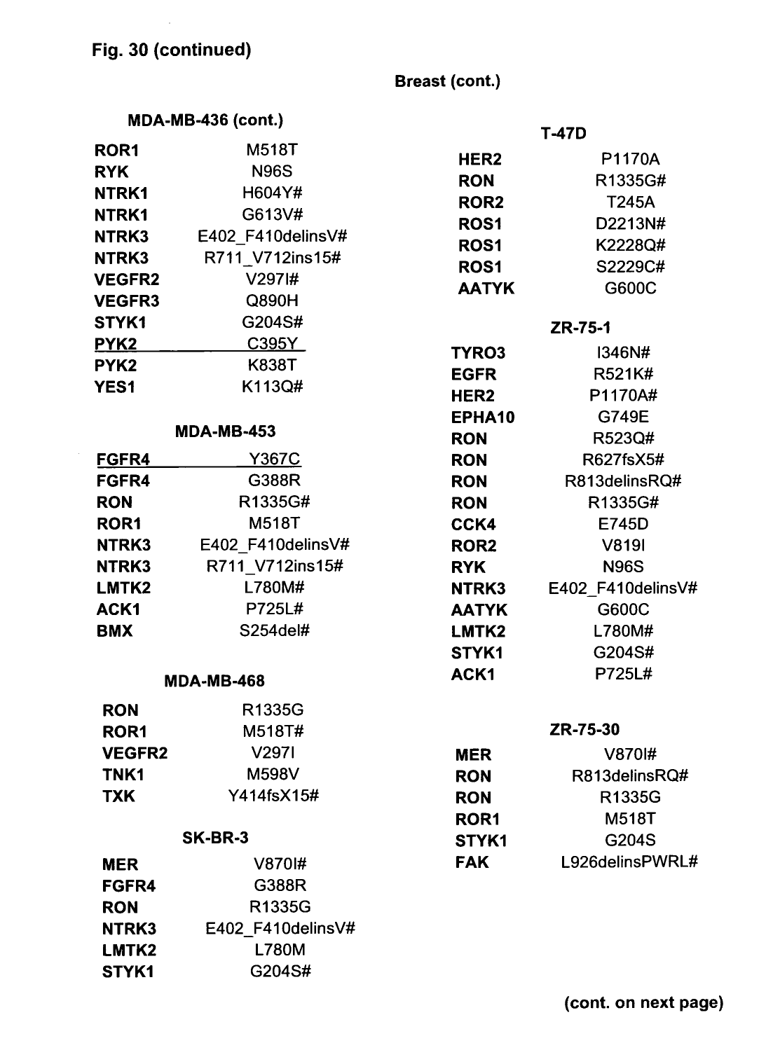

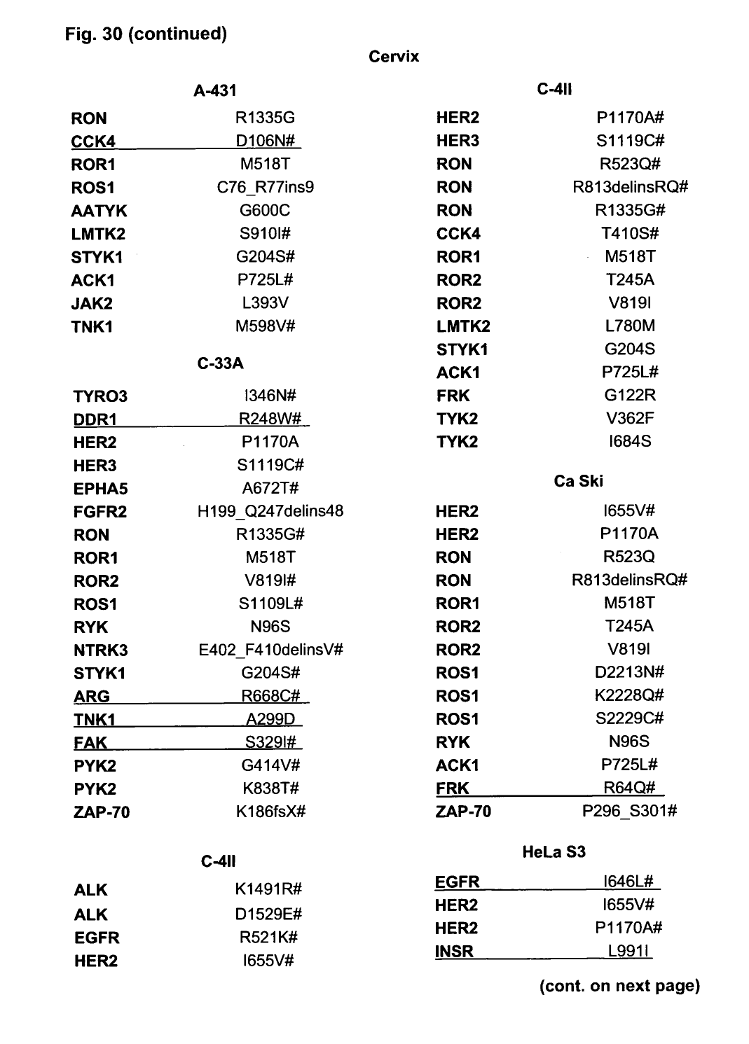

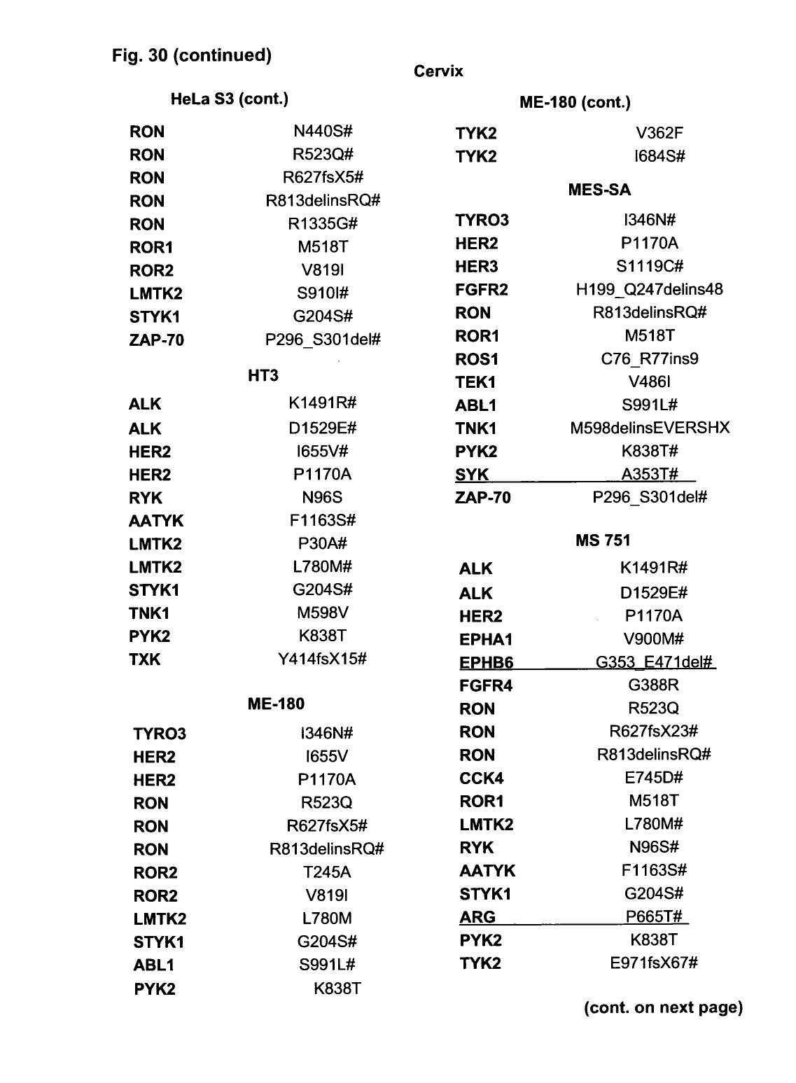

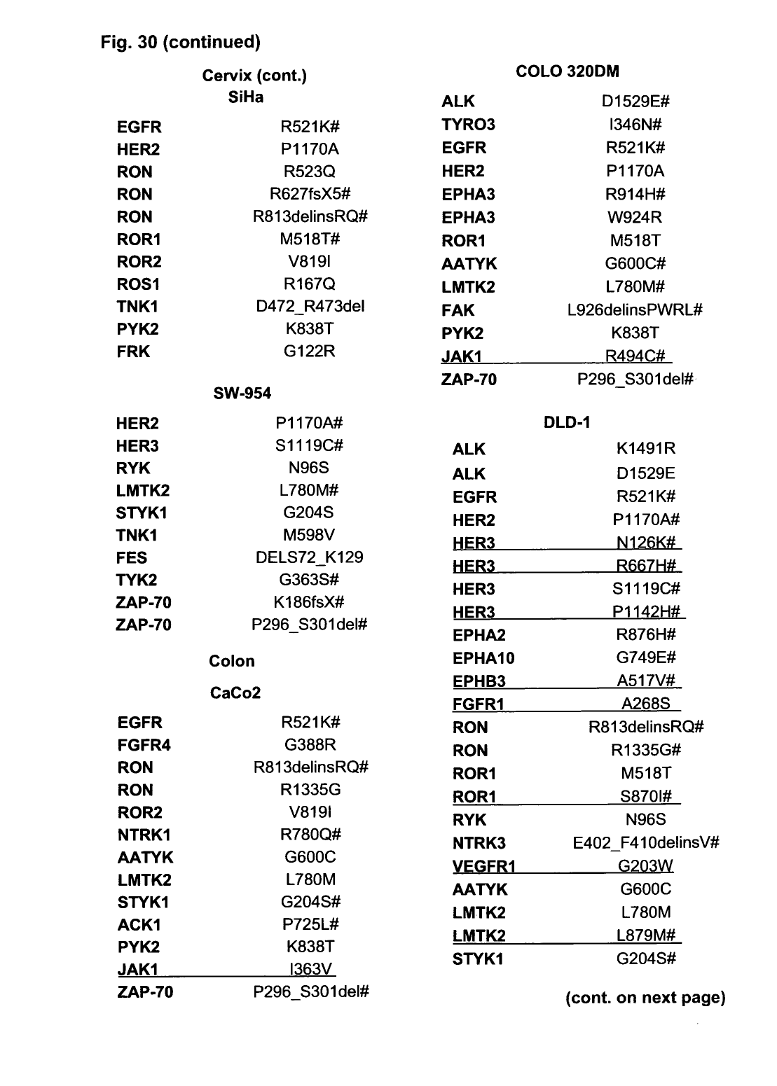

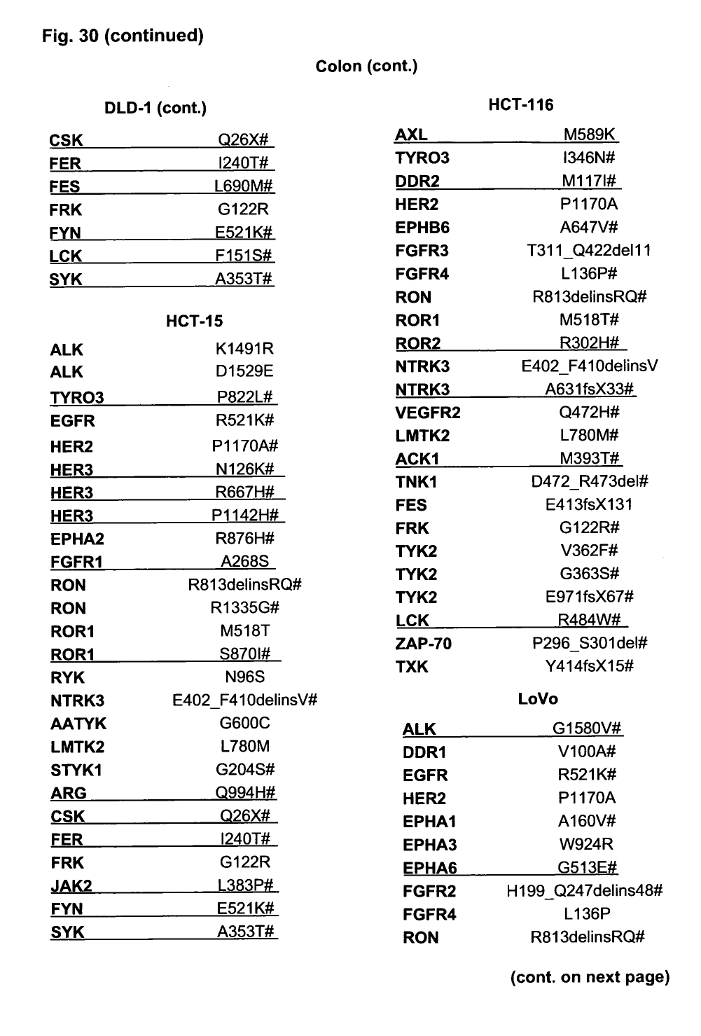

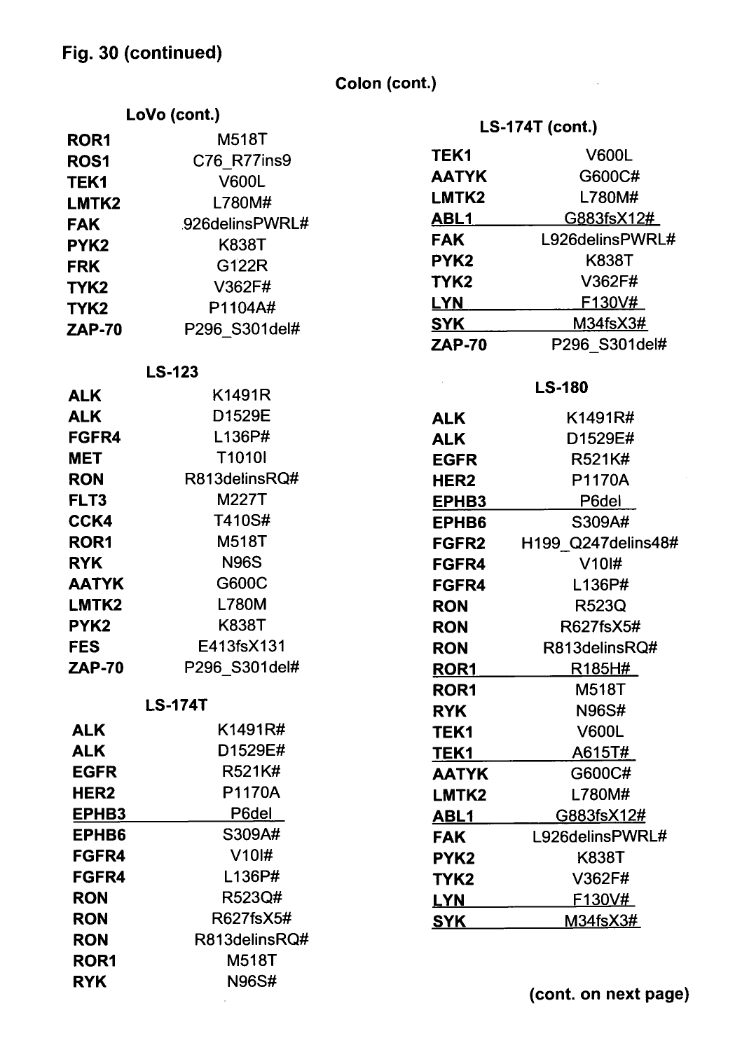

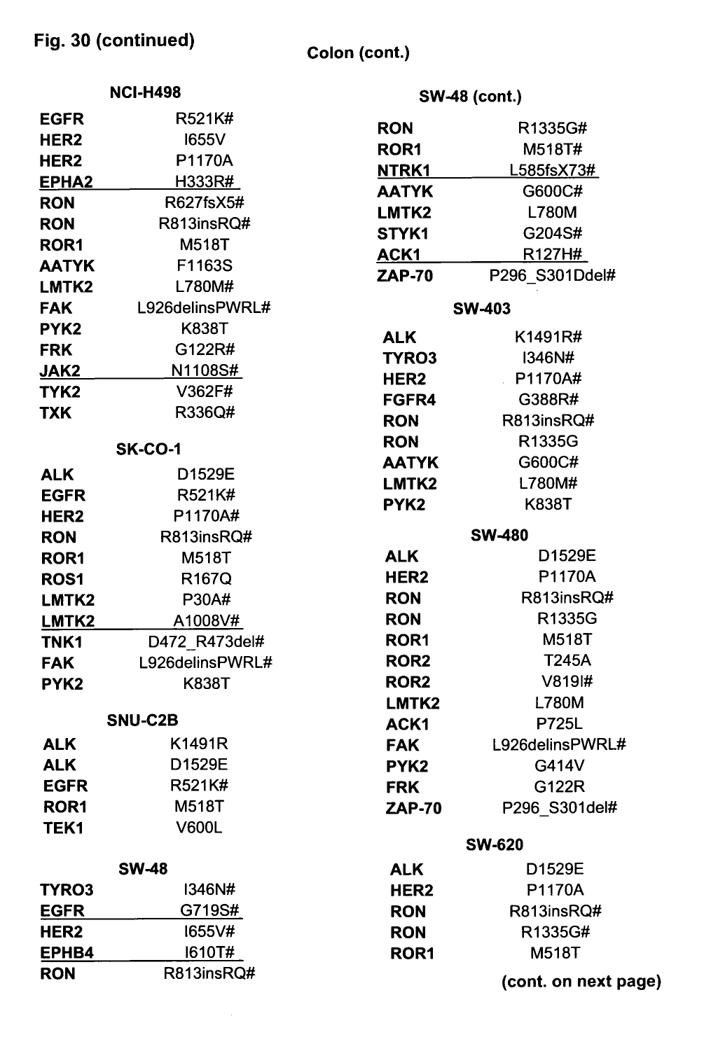

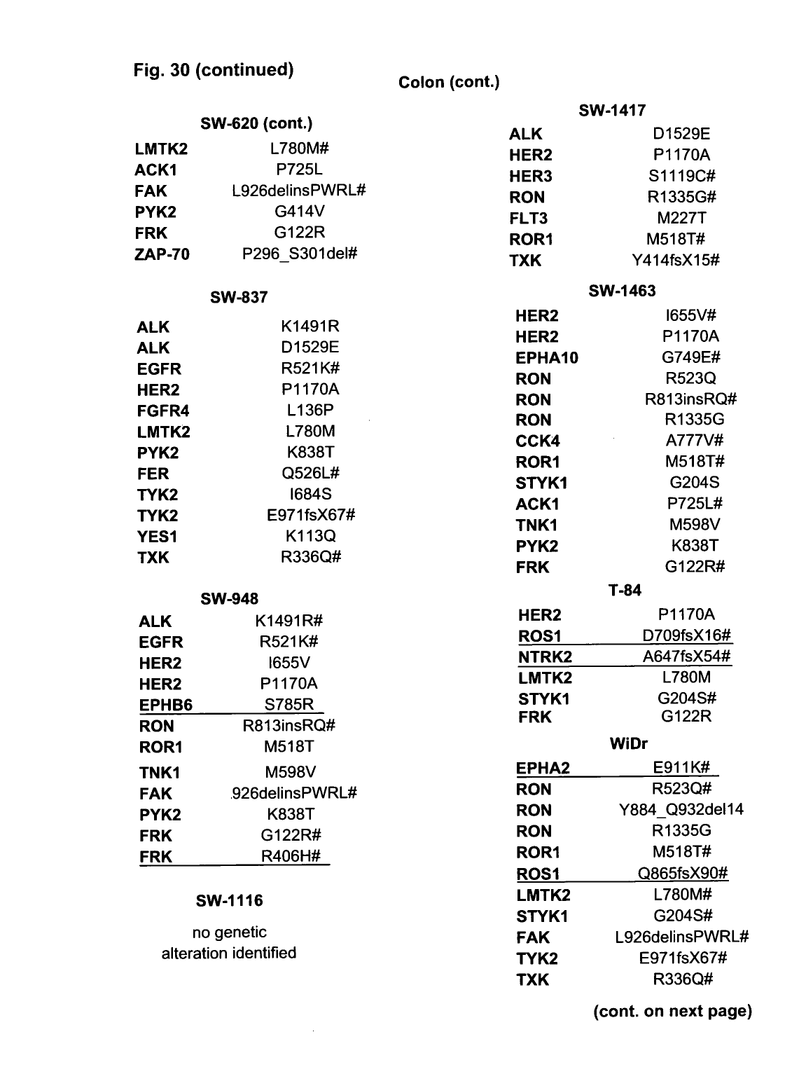

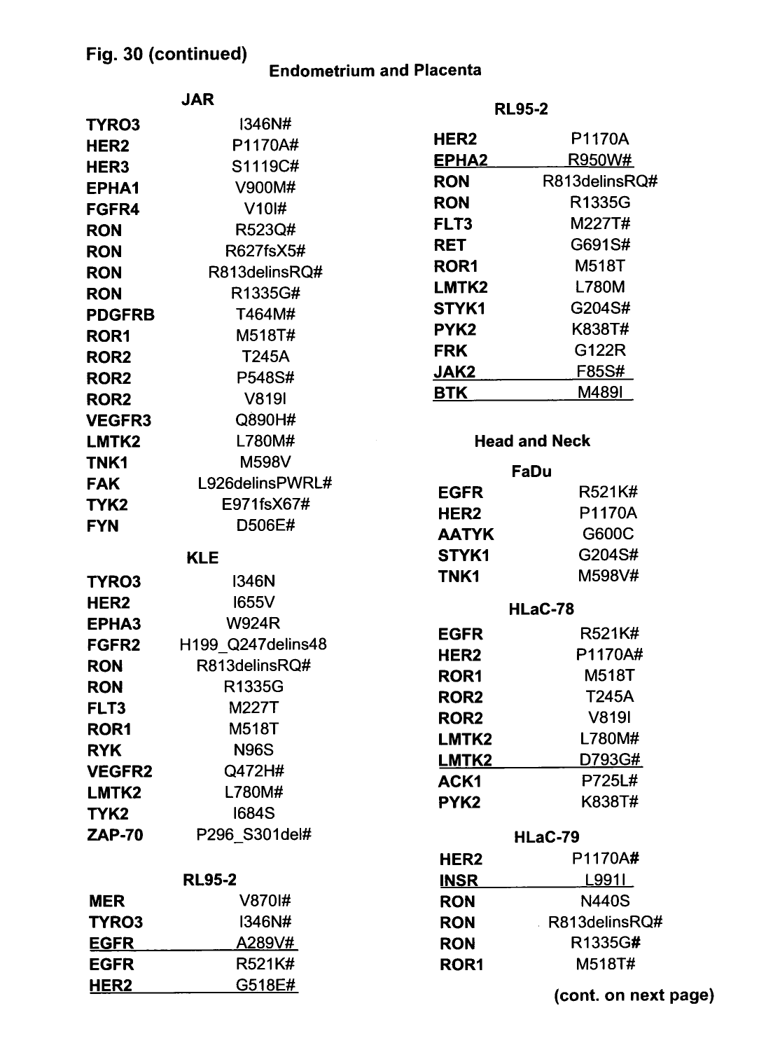

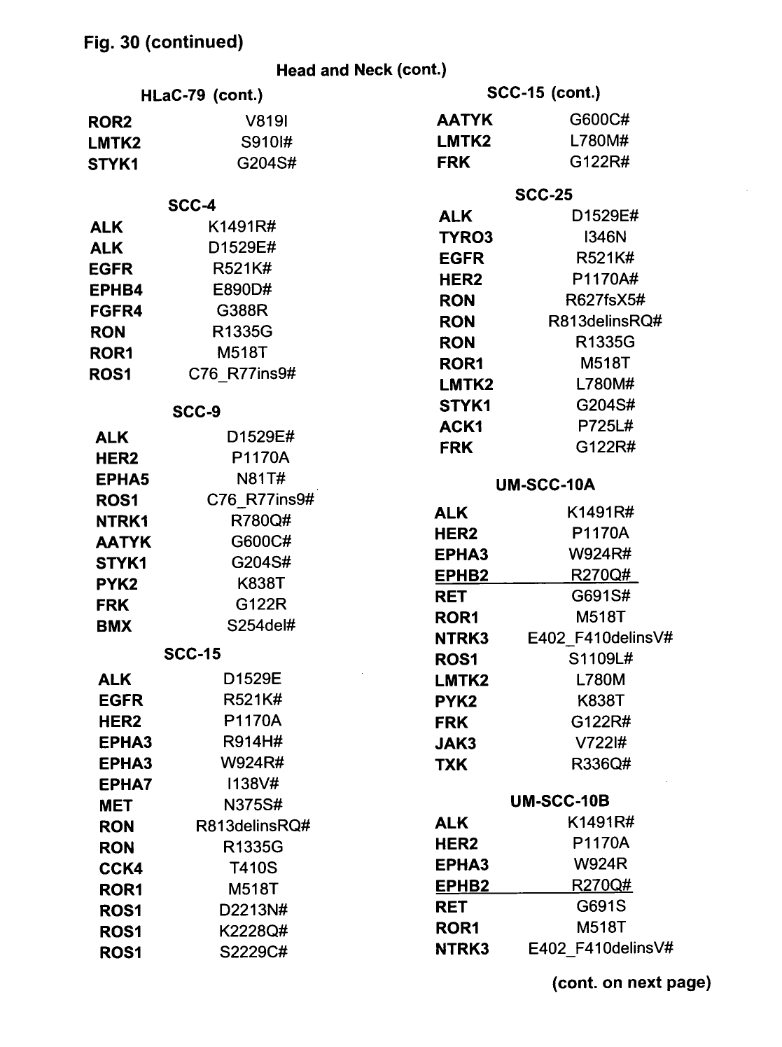

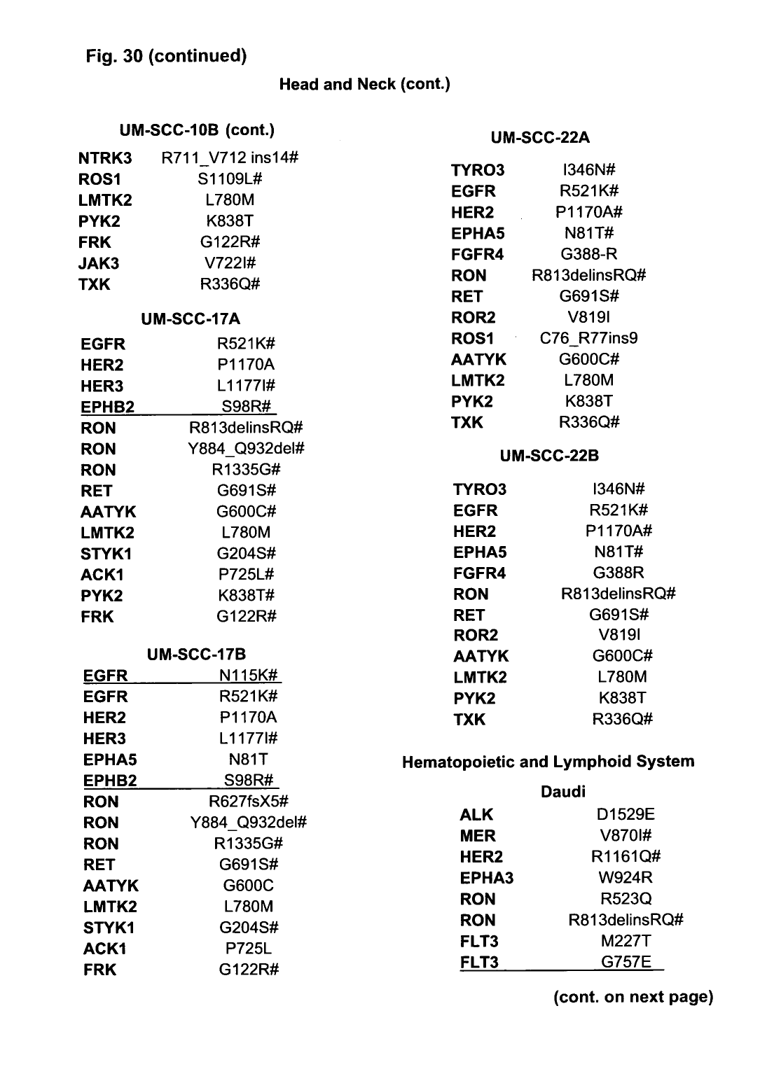

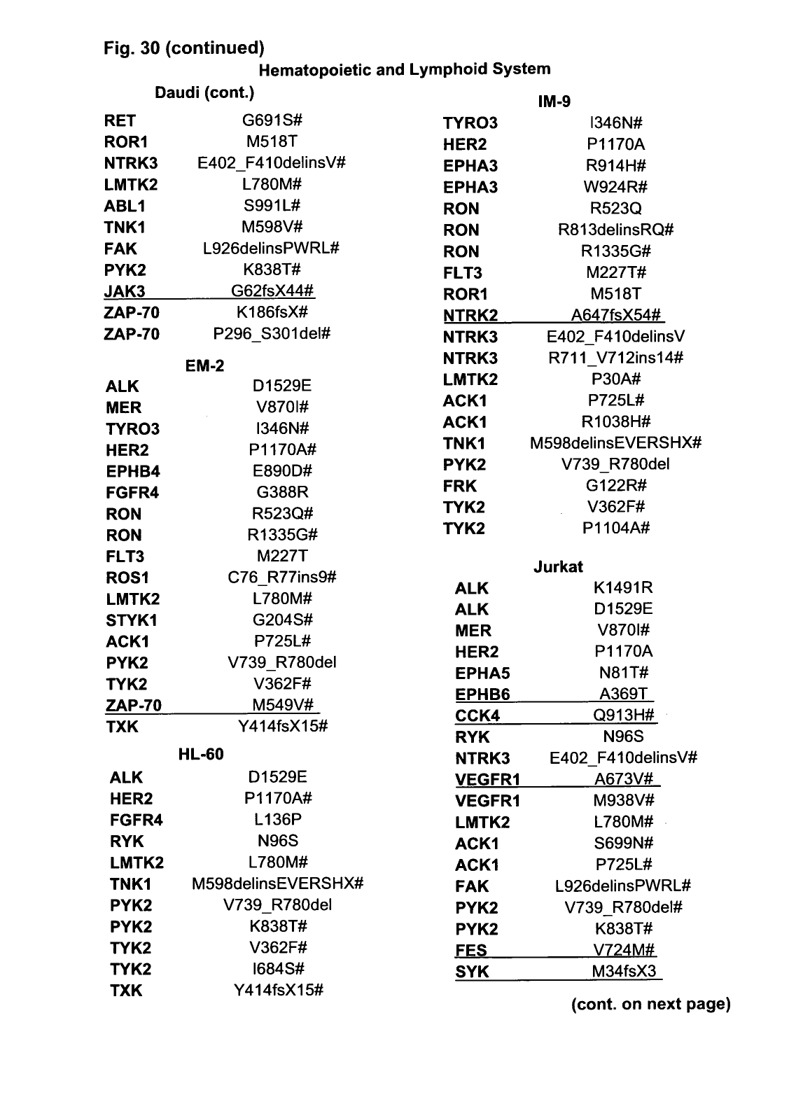

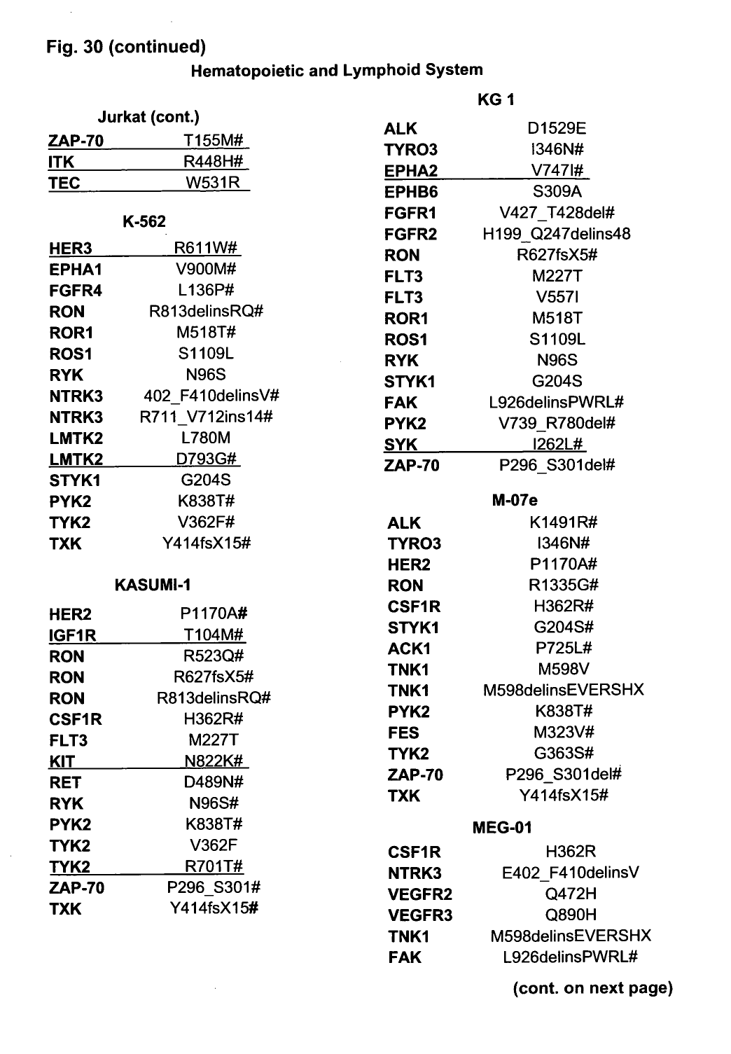

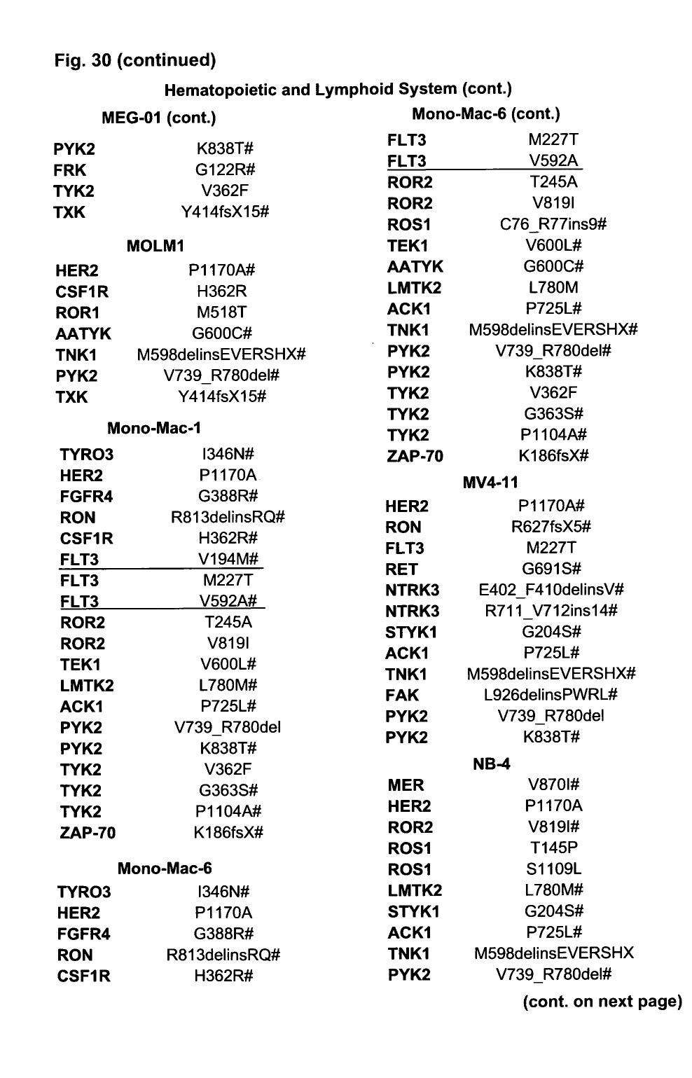

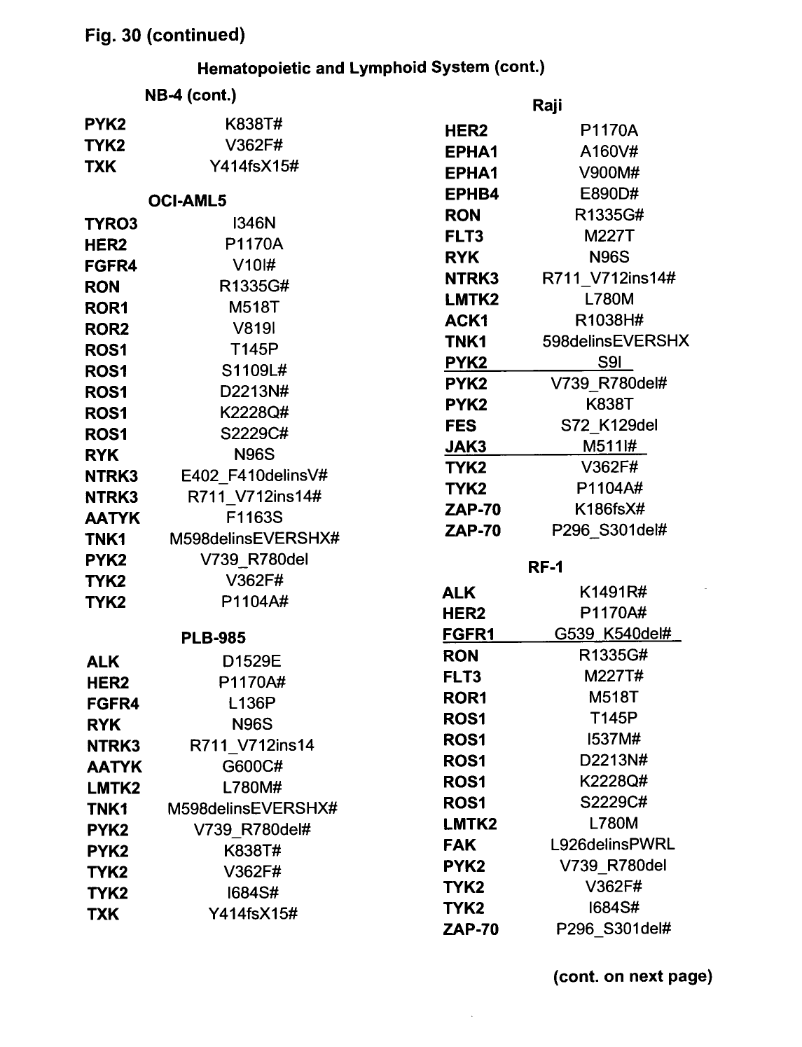

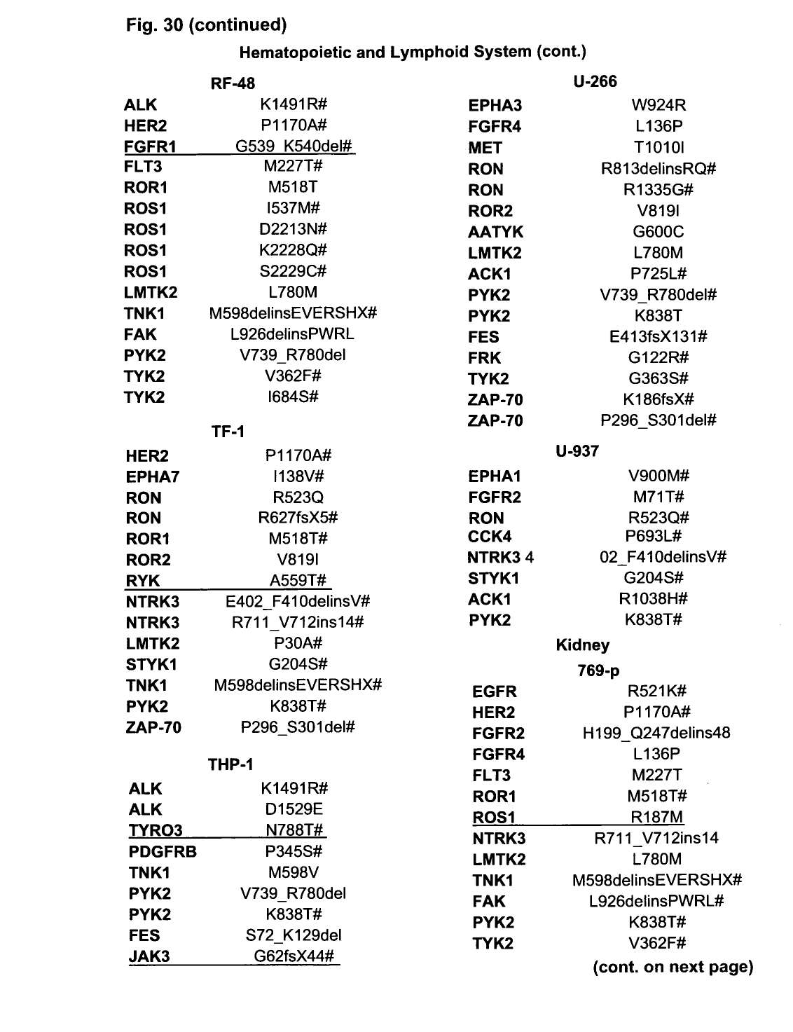

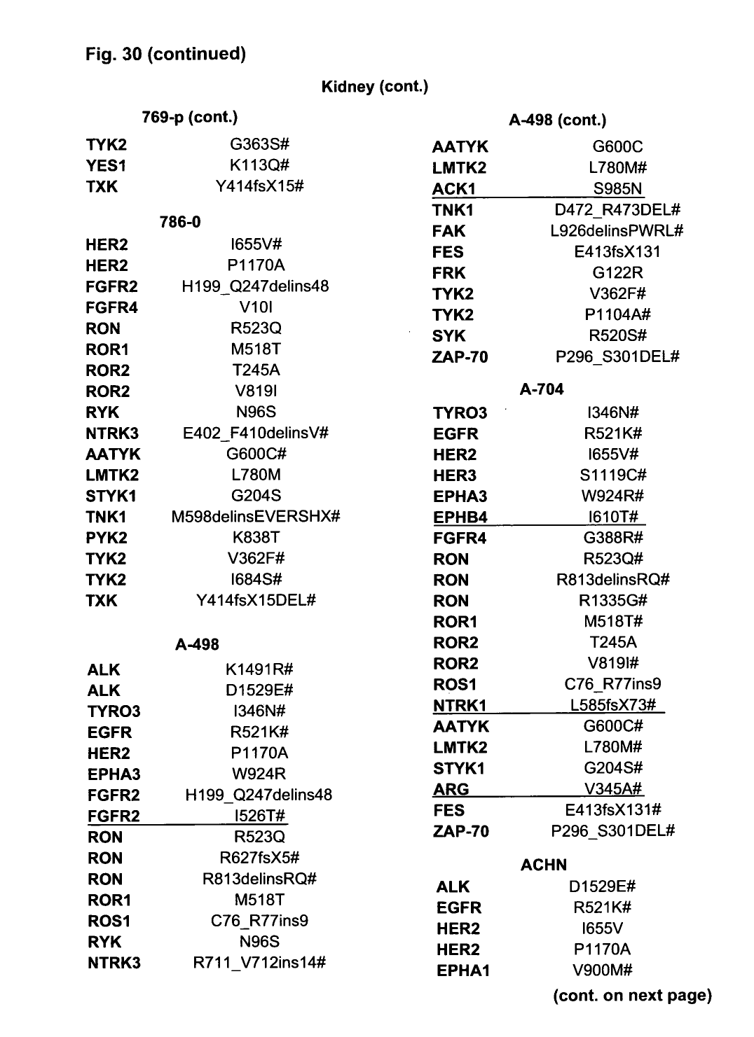

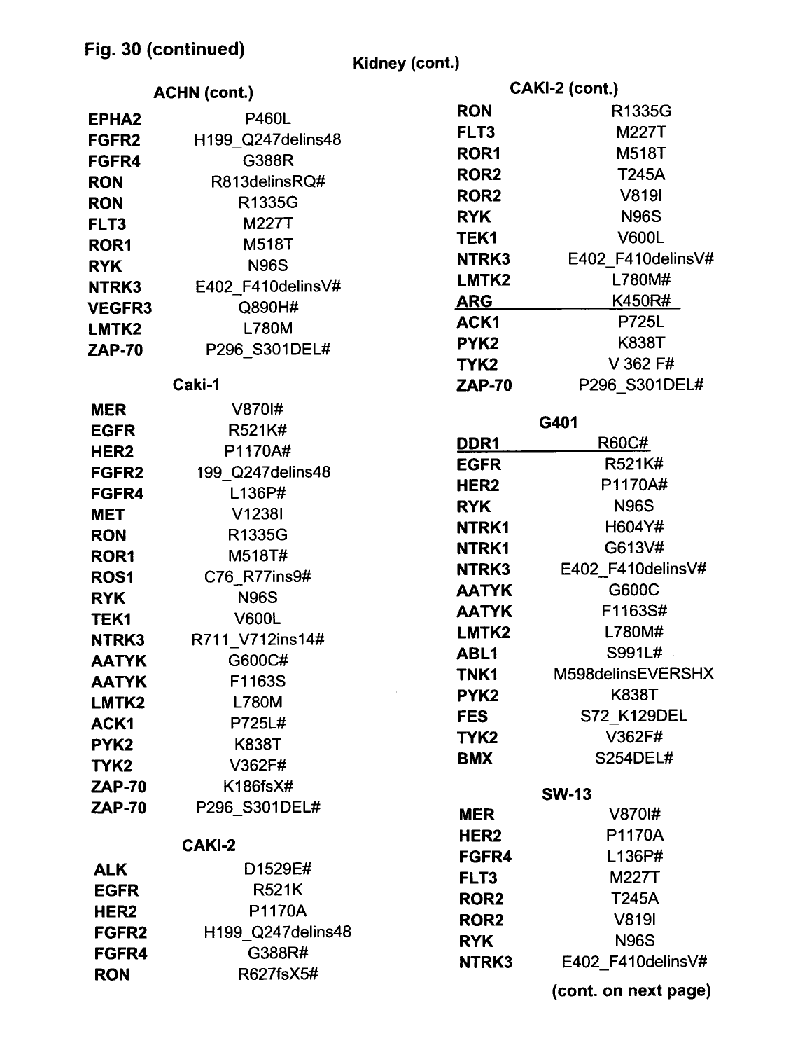

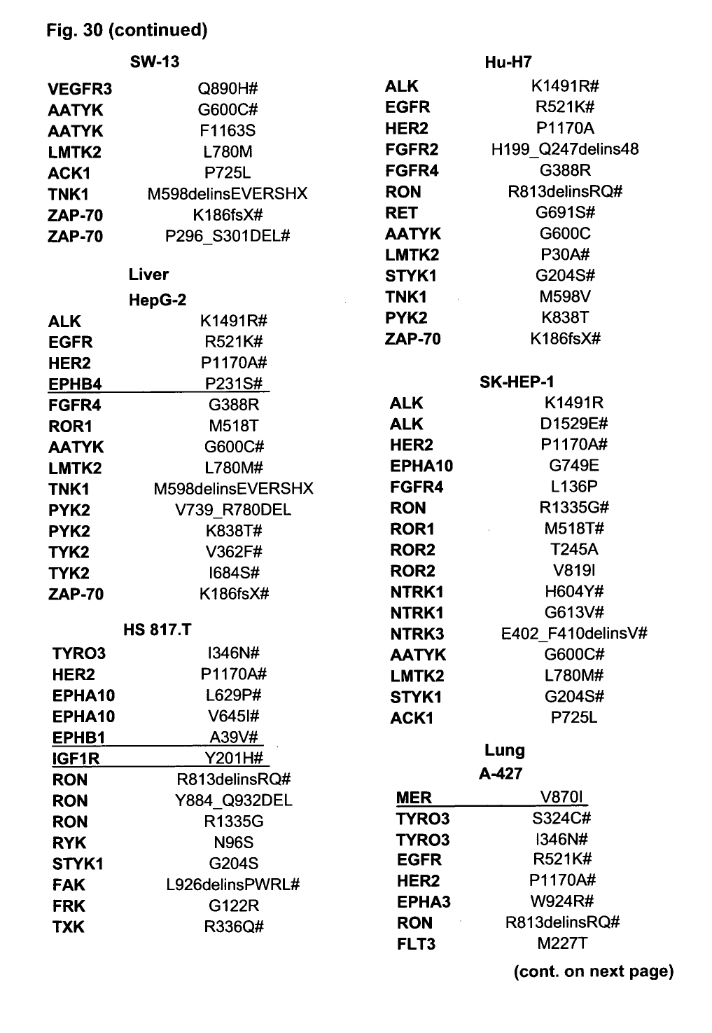

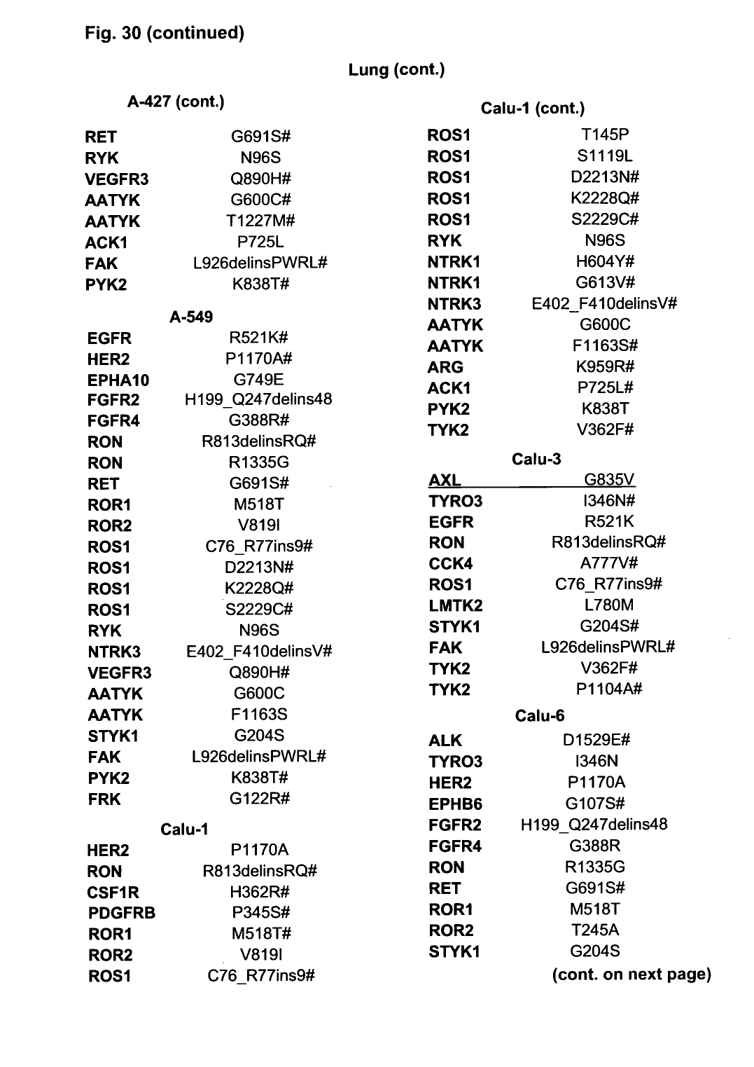

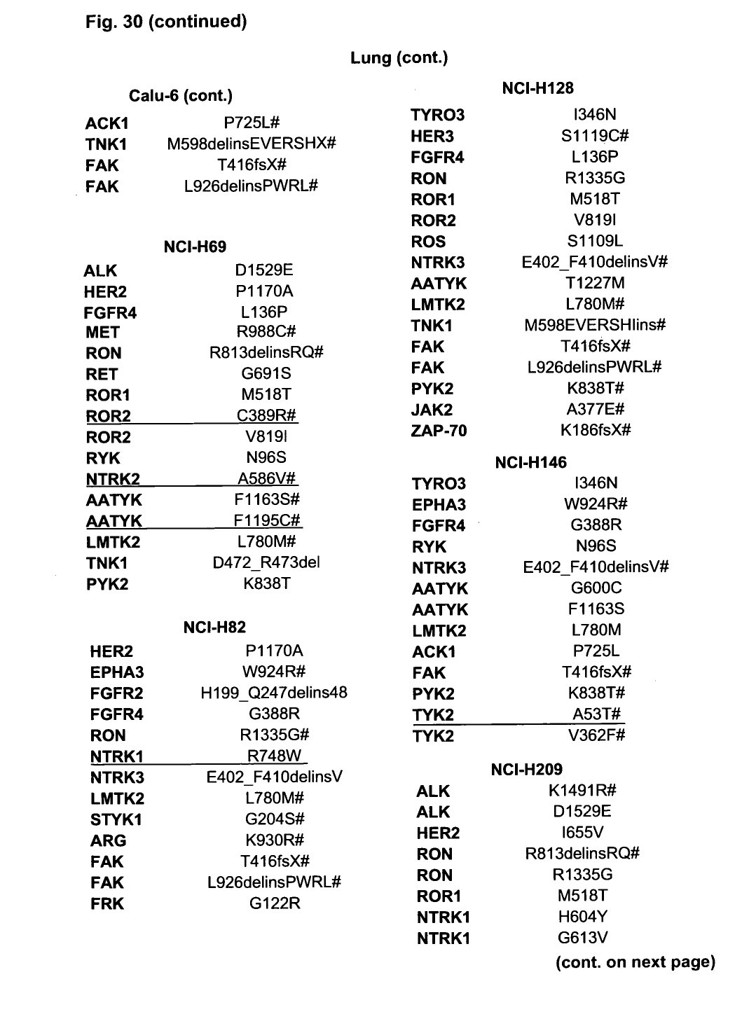

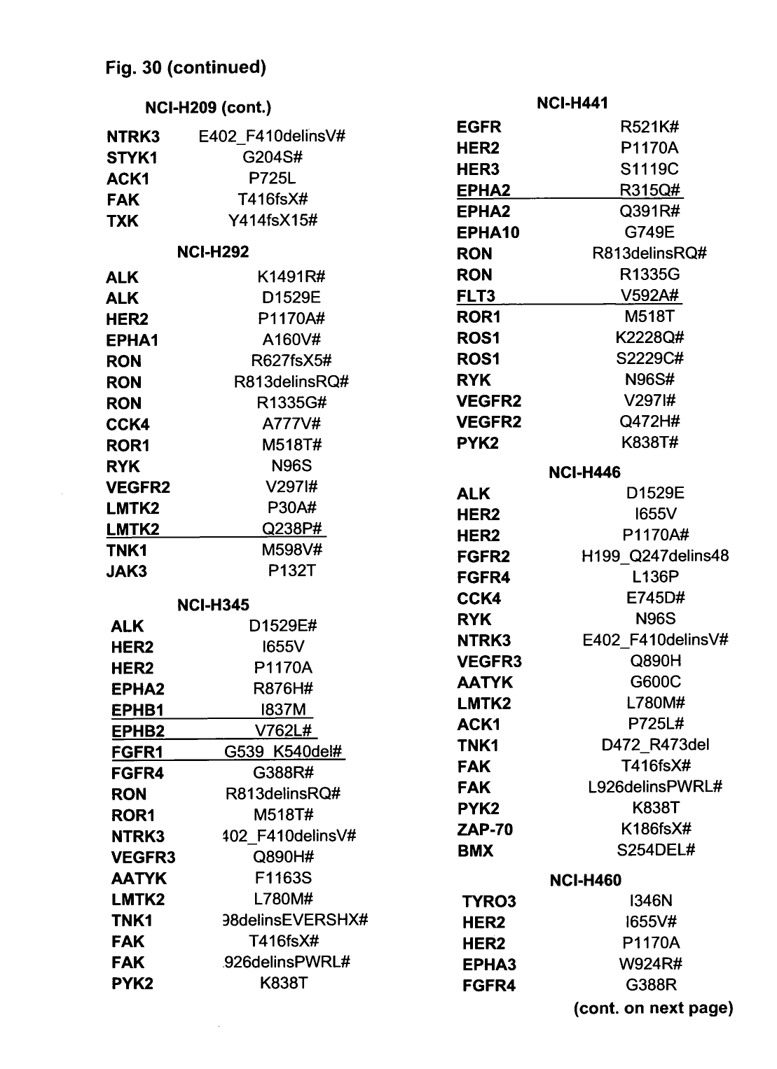

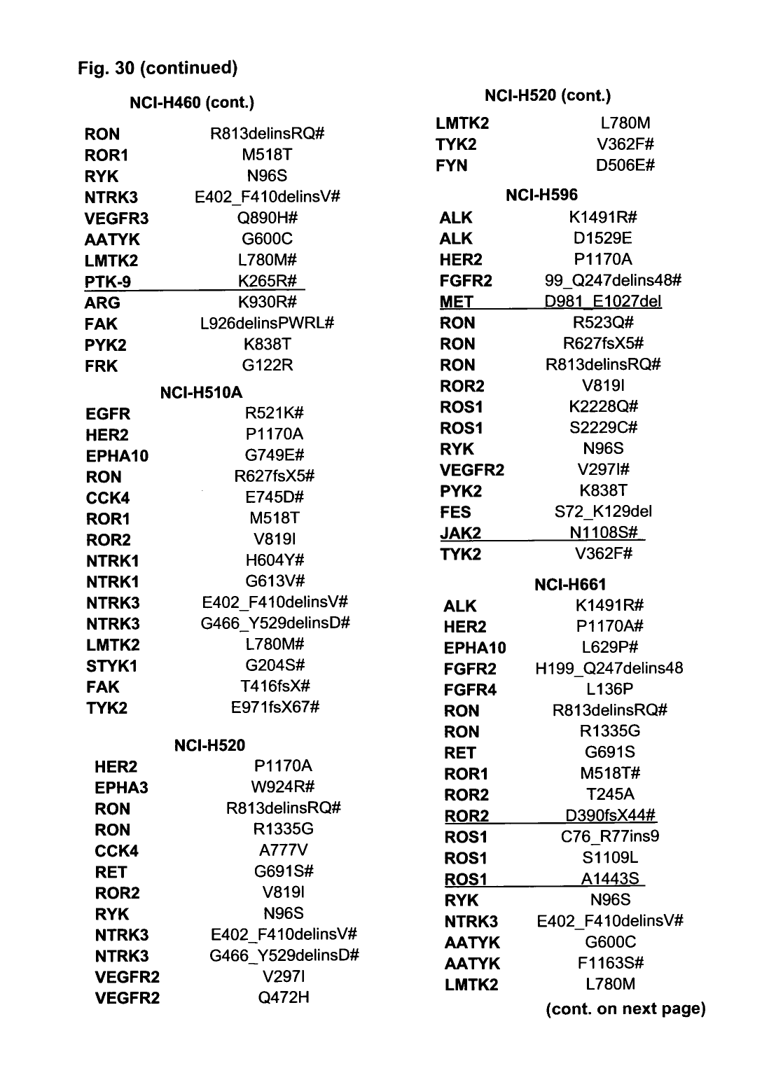

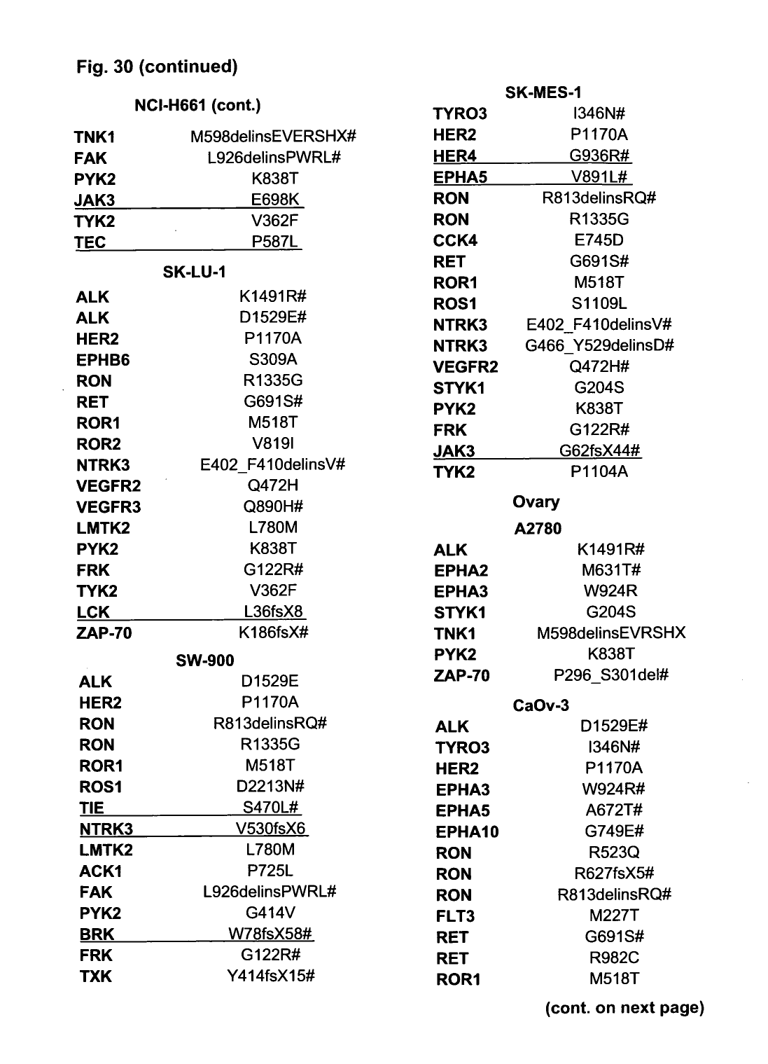

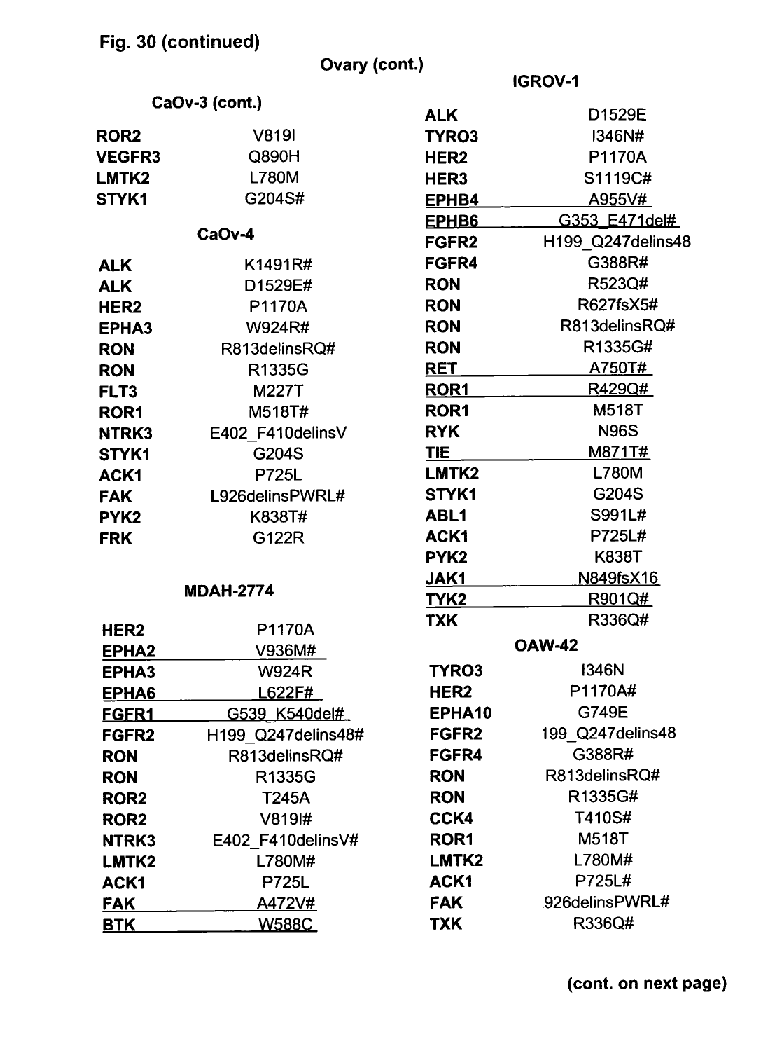

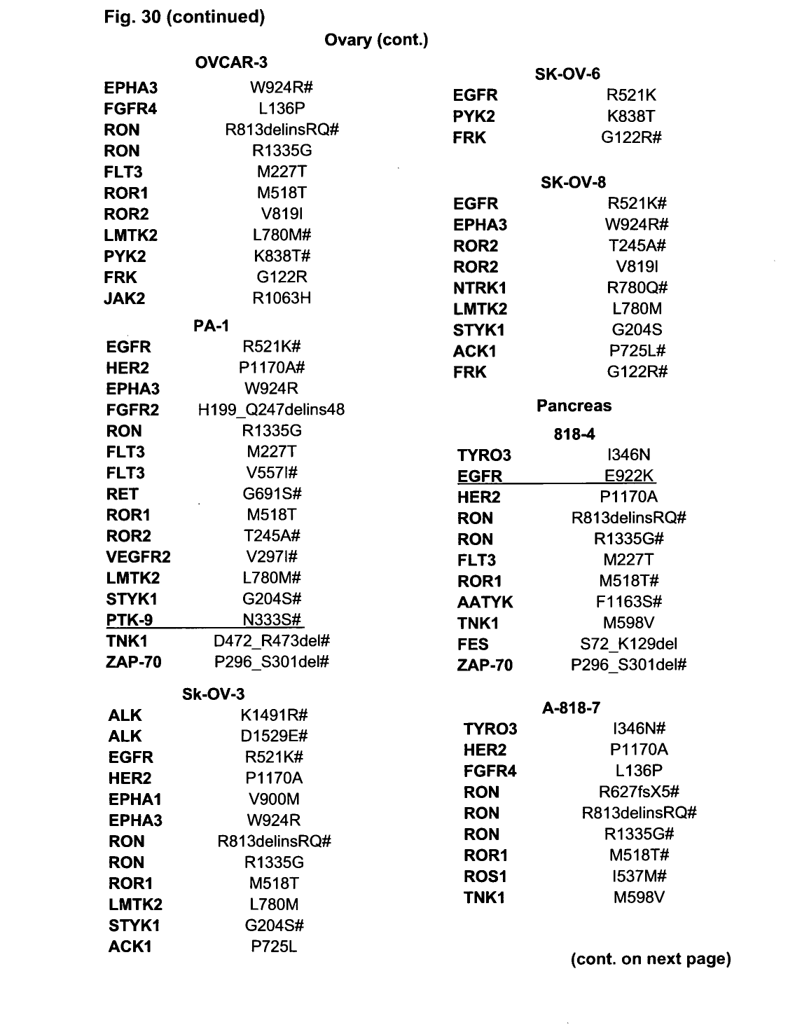

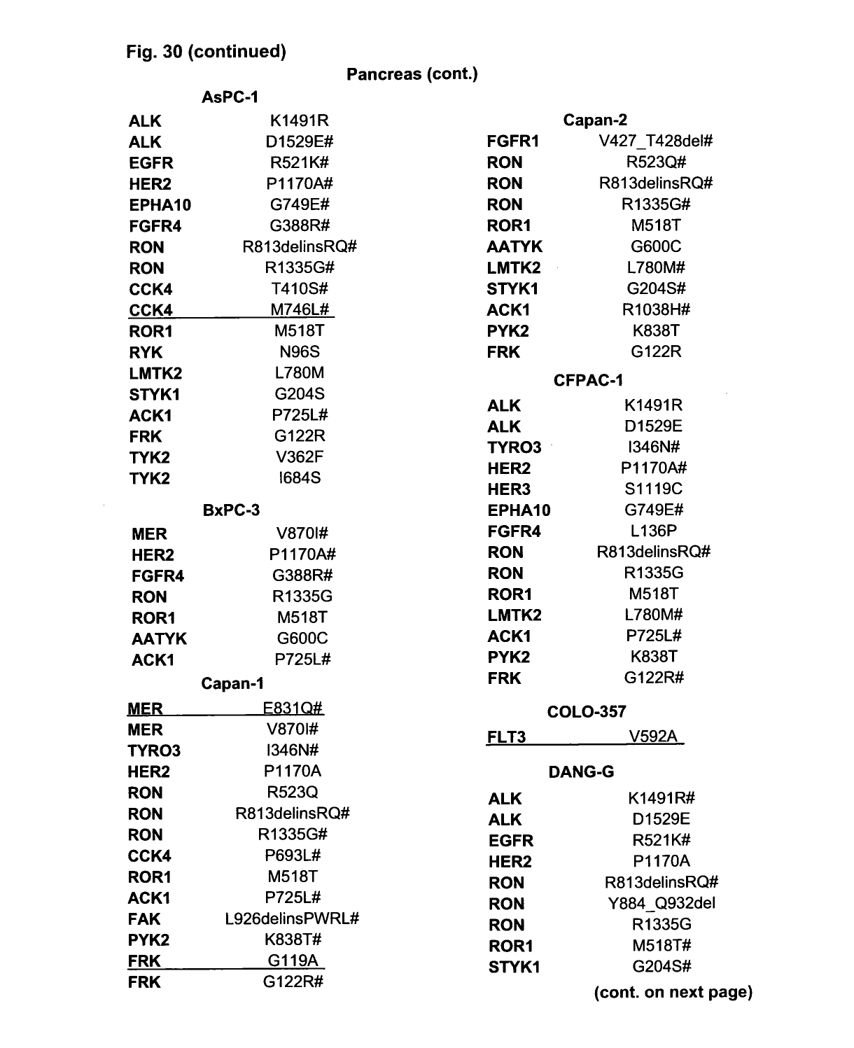

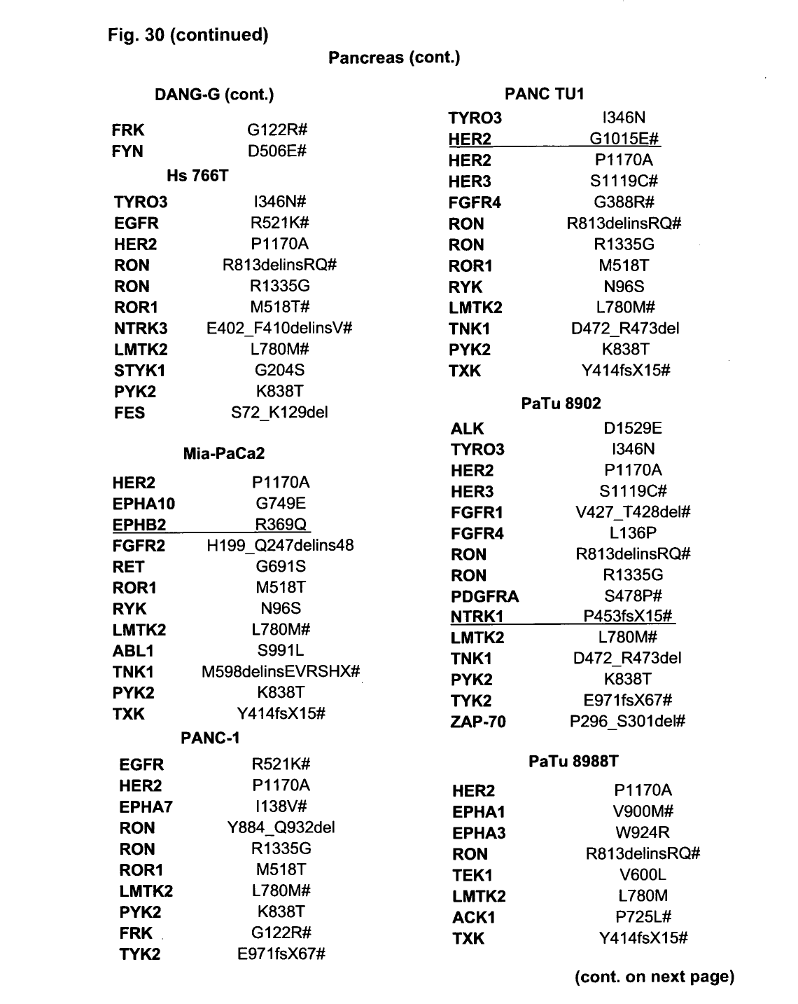

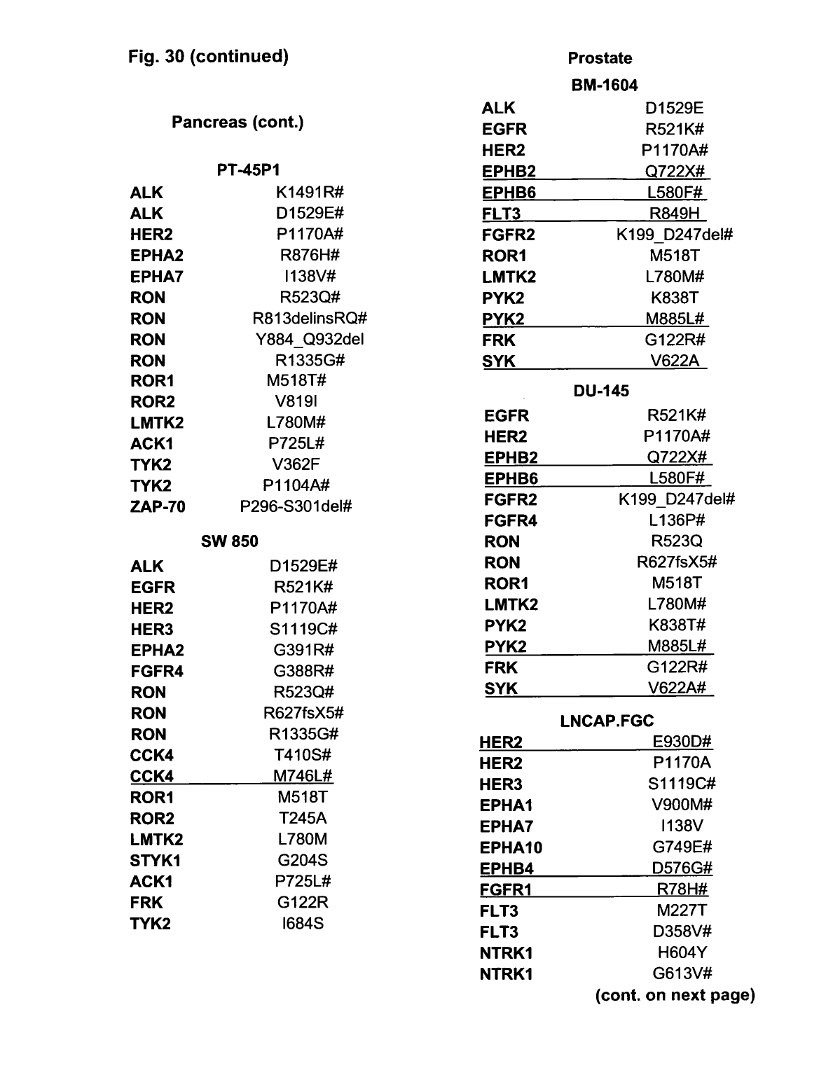

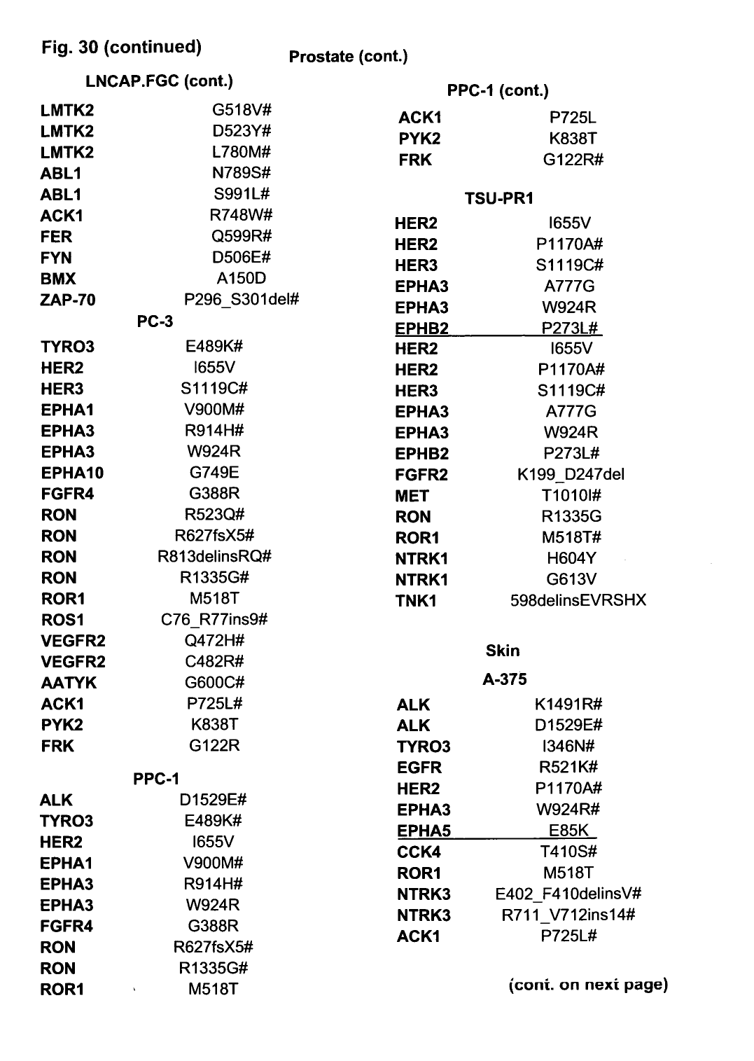

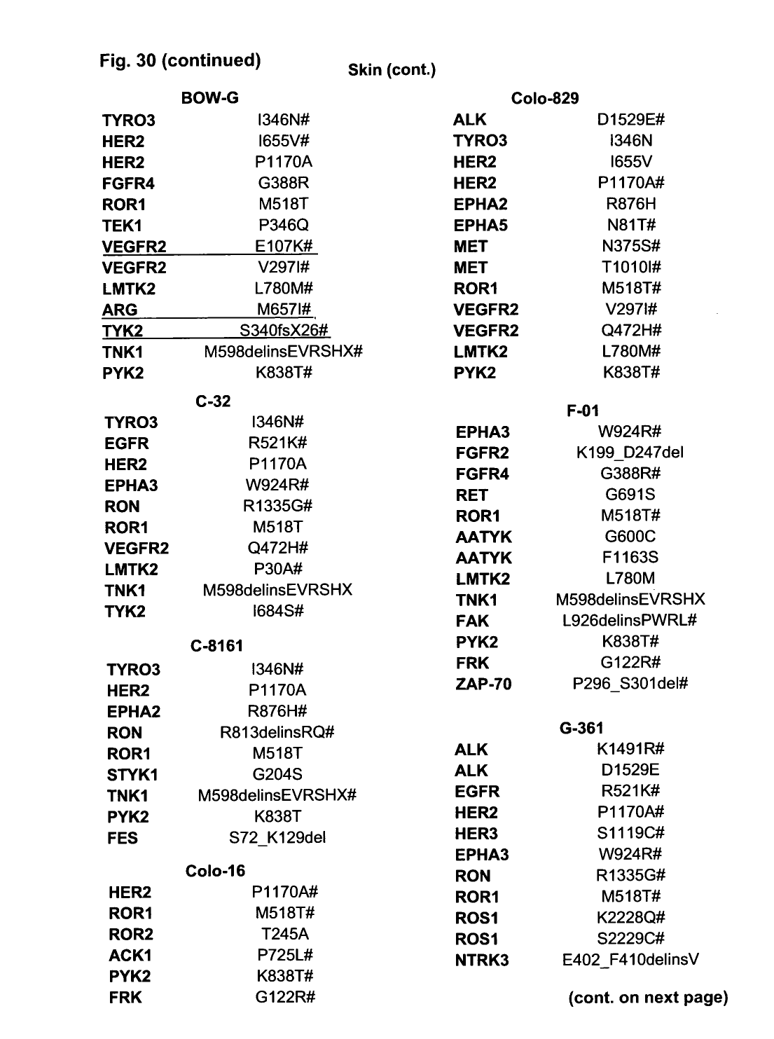

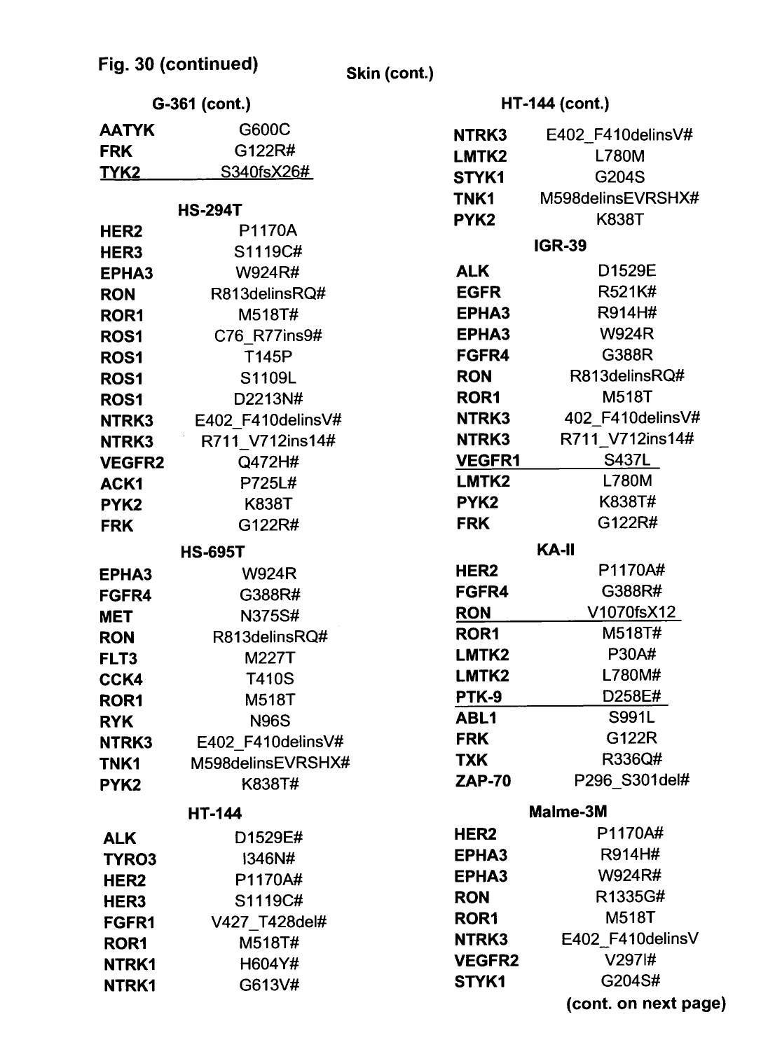

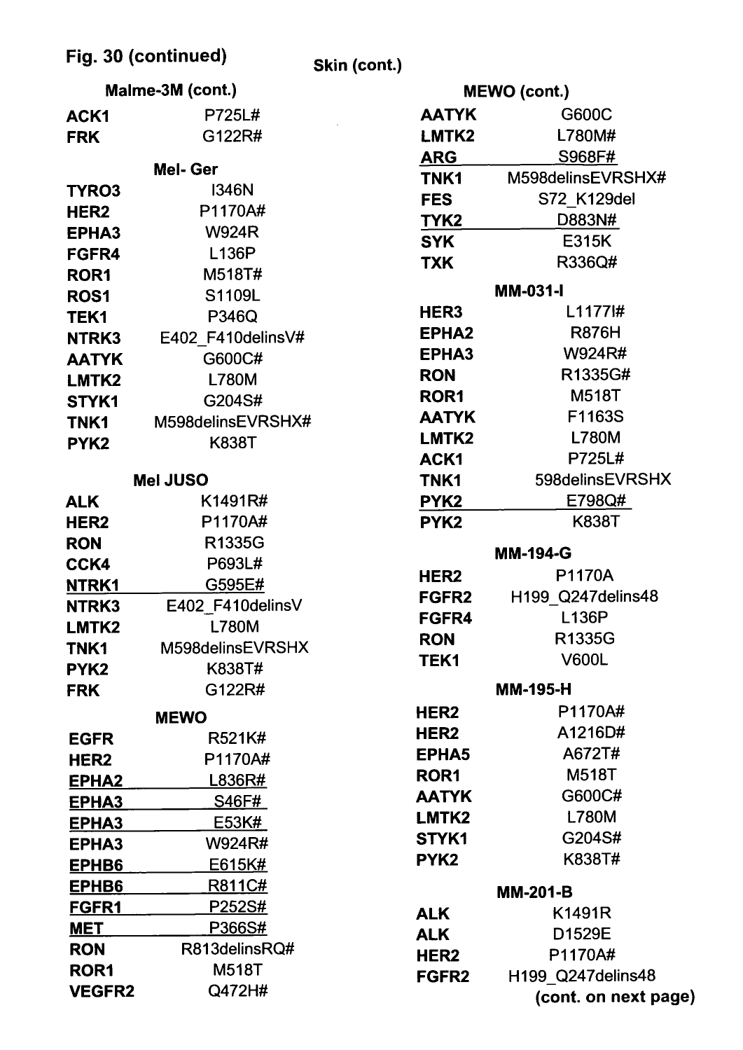

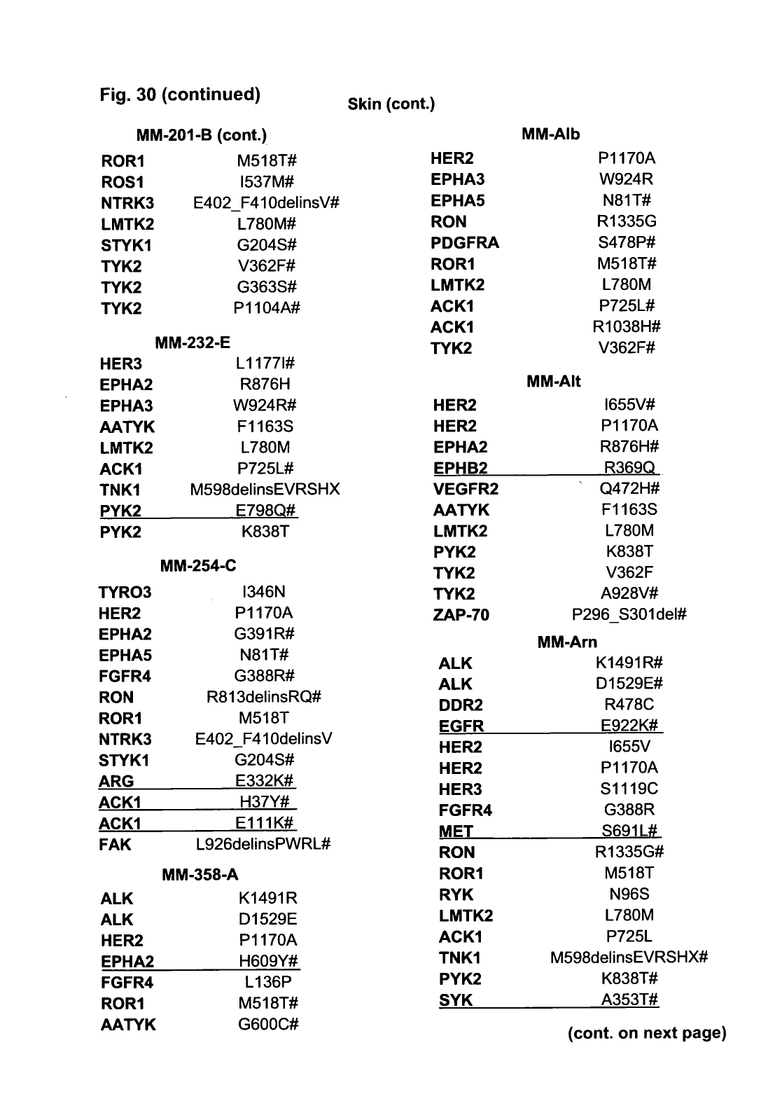

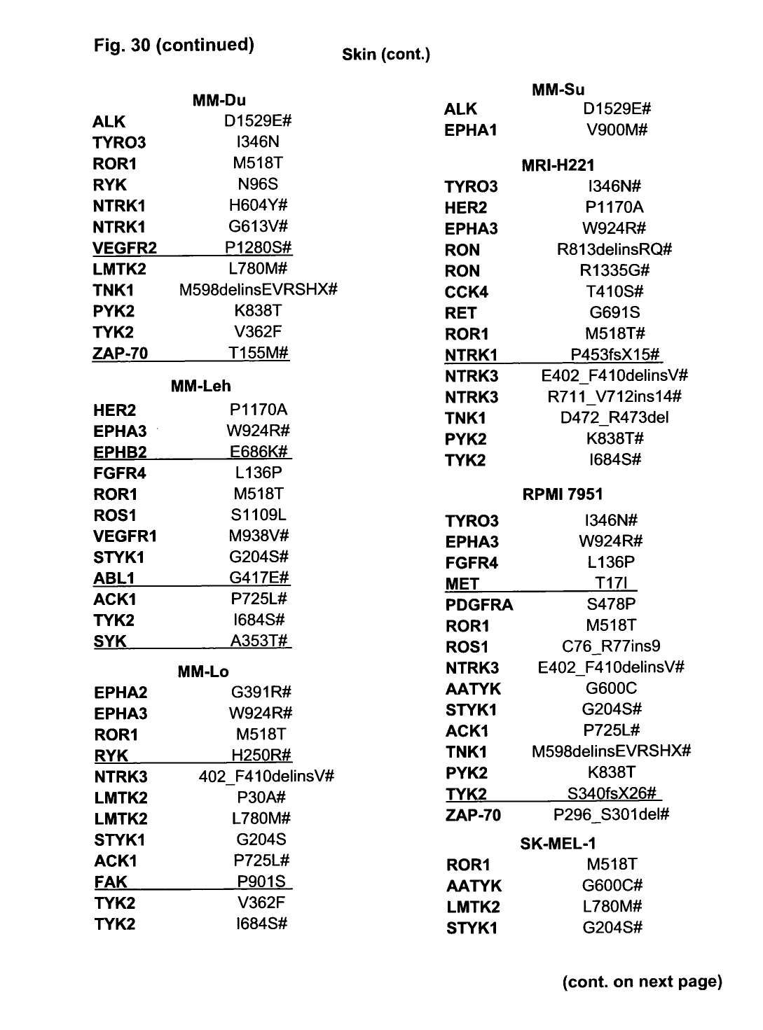

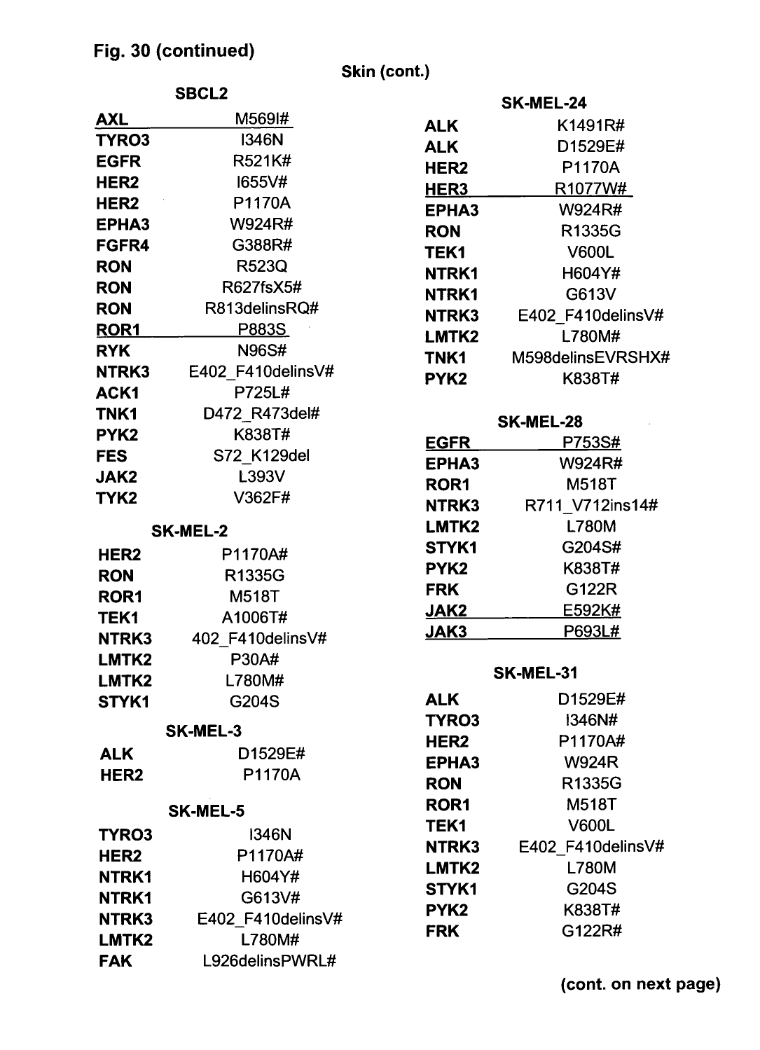

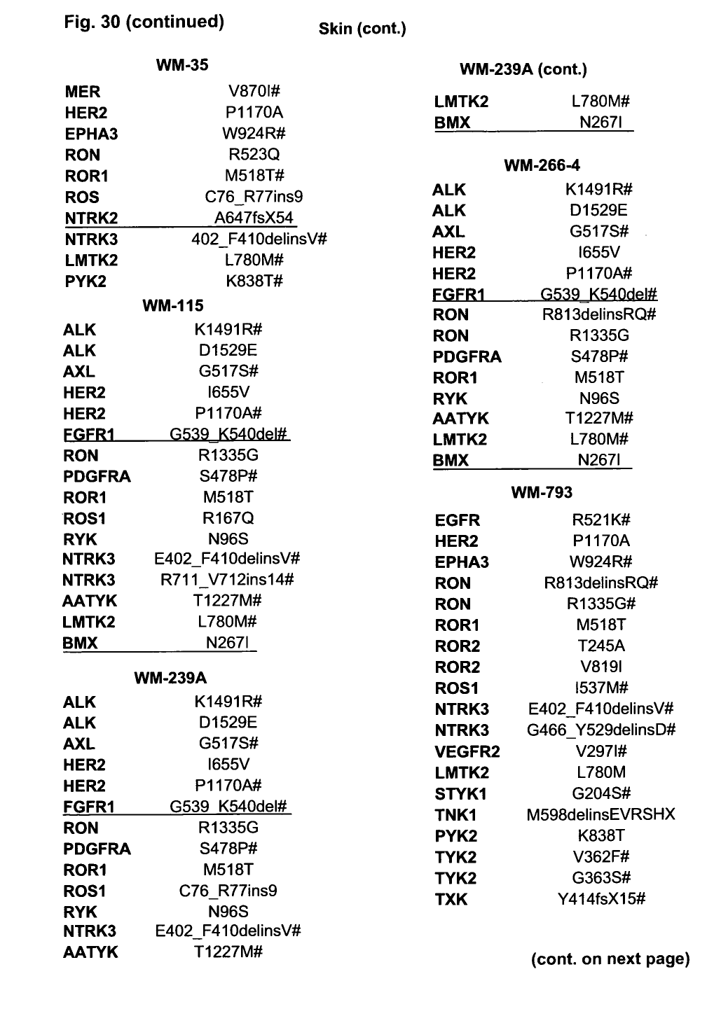

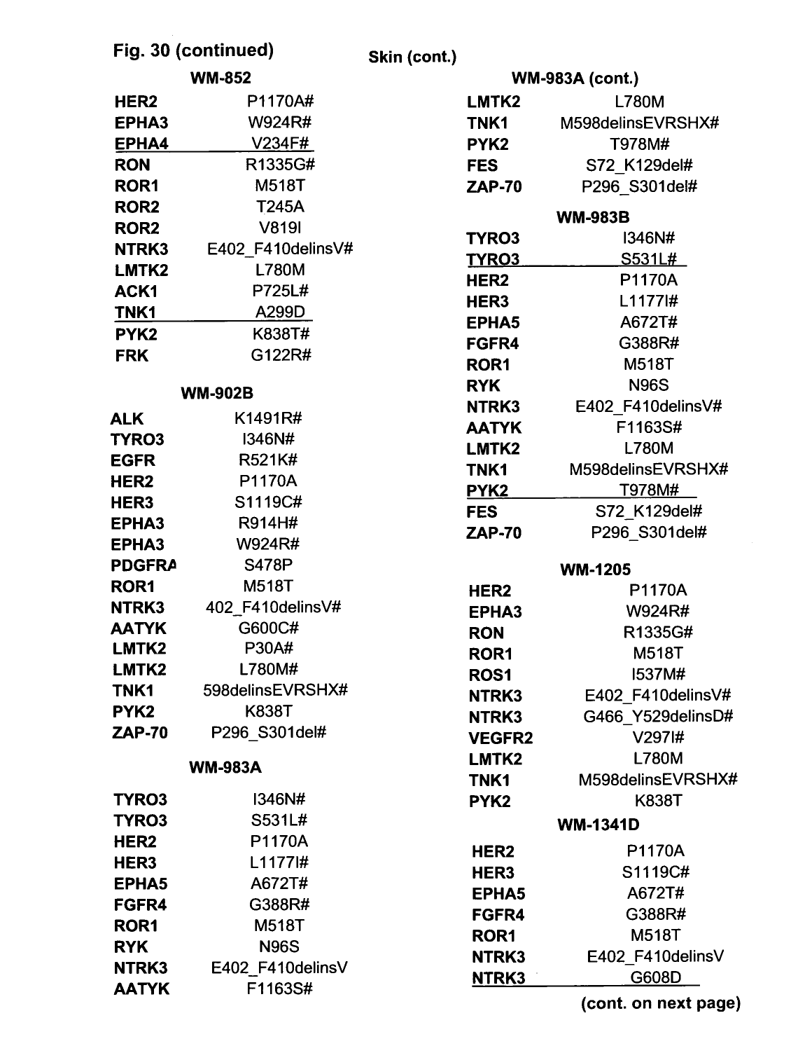

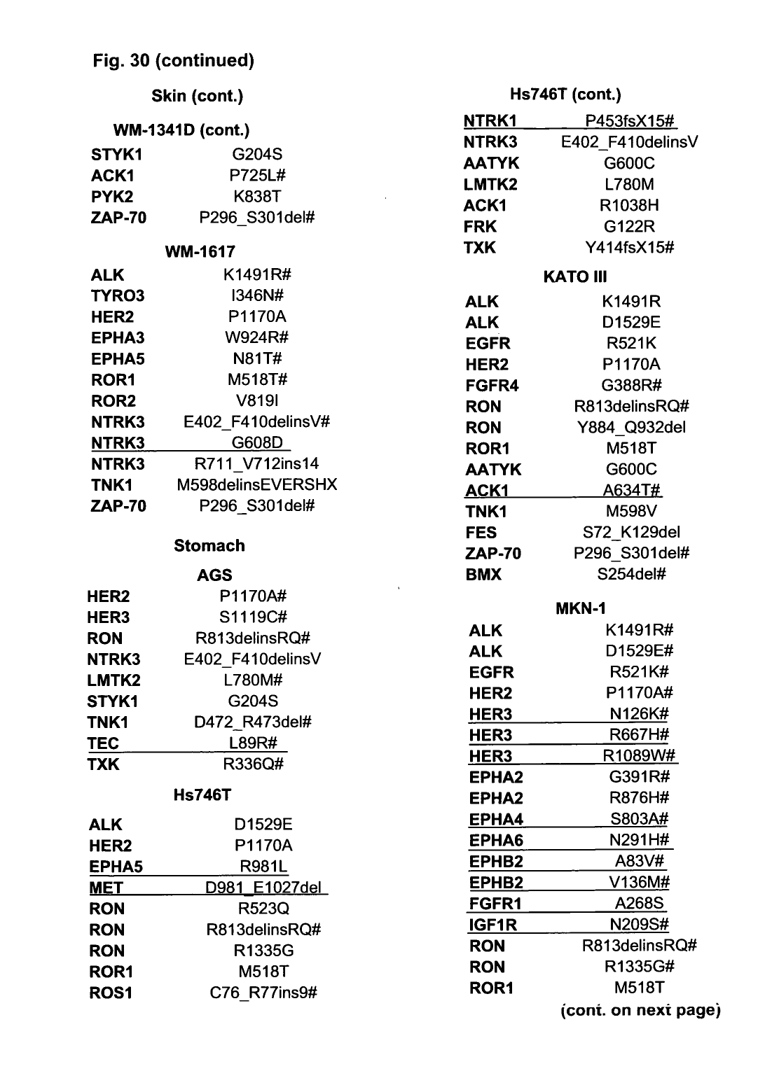

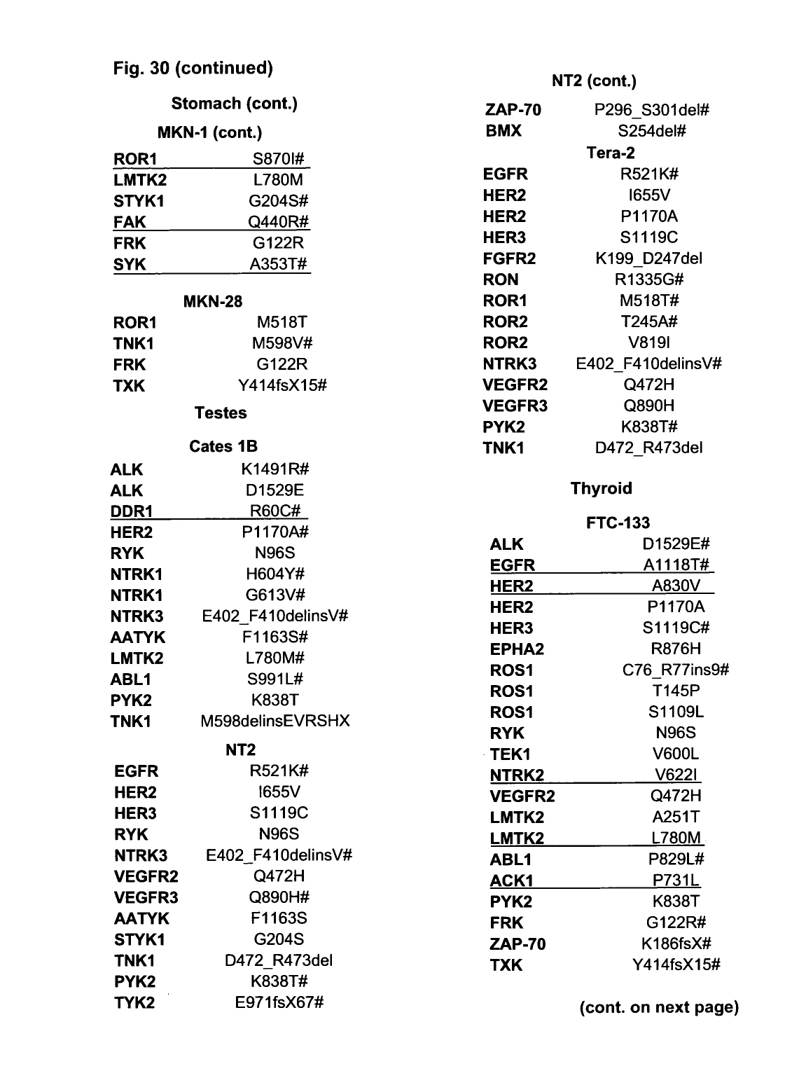

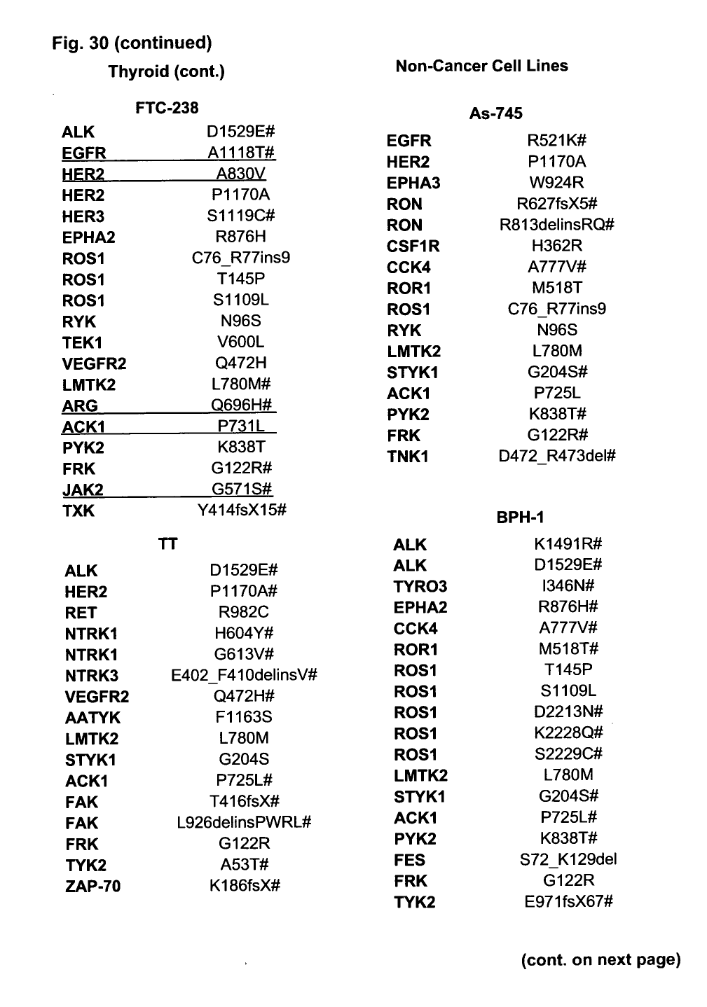

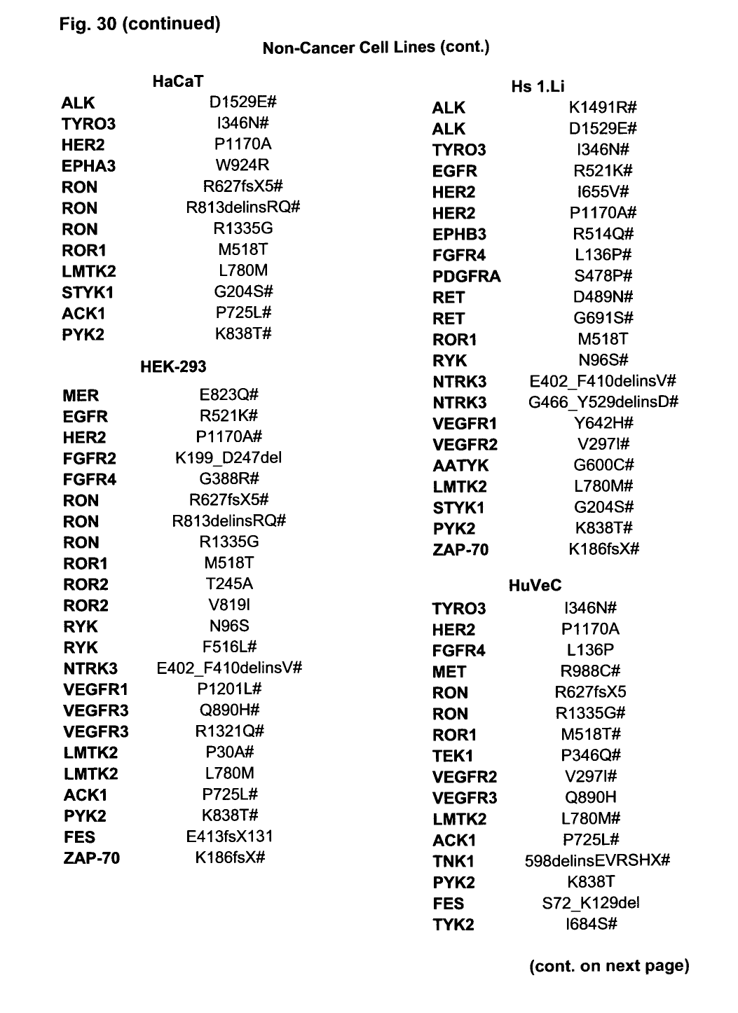

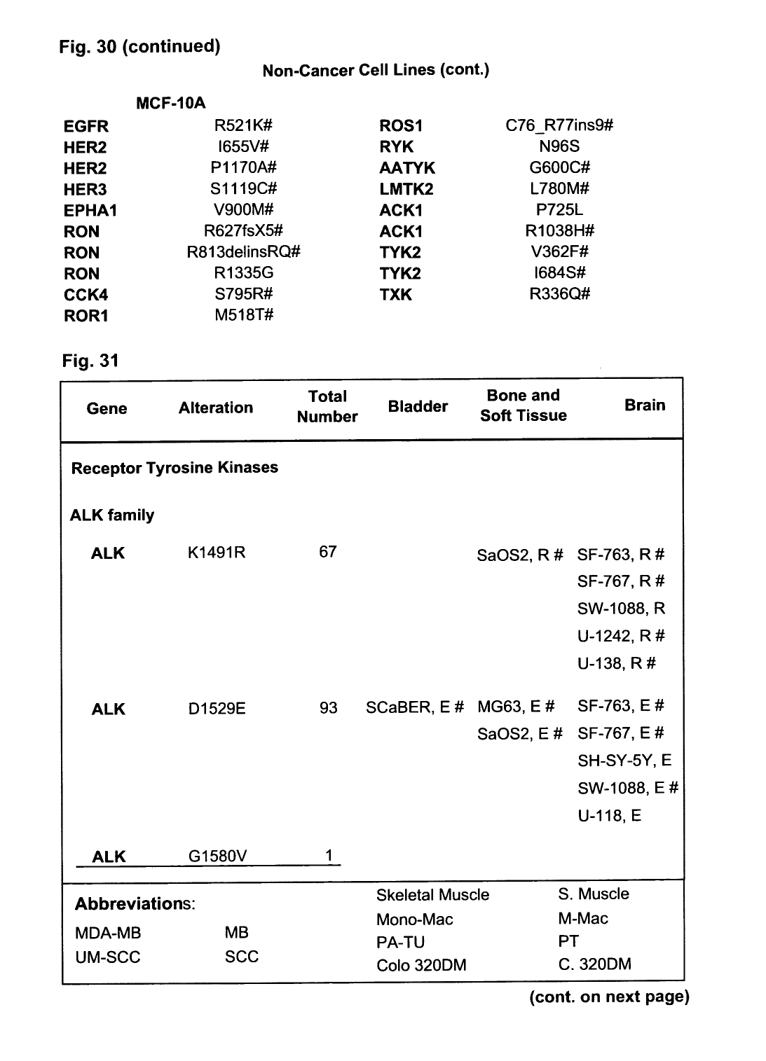

FIG. 1 depicts a characterization of tumor cell lines with regard to genetic alterations in the tyrosine kinase transcriptome (TKT). FIG. 1A: samples. The tissue origins and number of tumor cell lines derived thereof are summarized. FIG. 1B: patterns of genetic alterations. For each of the 254 tumor and 7 control cell lines, the specific pattern of non-synonymous genetic alterations within the tyrosine kinase transcriptome is provided (FIG. 30) and exemplarily shown for 5 skin-derived tumor cell lines. Germline polymorphisms and somatic mutations are highlighted in blue and yellow, respectively. FIG. 1C: genetic alterations per TKT. The number of tumor cell lines with the indicated number of somatic (light) or germline (dark) alterations detected therein is illustrated.

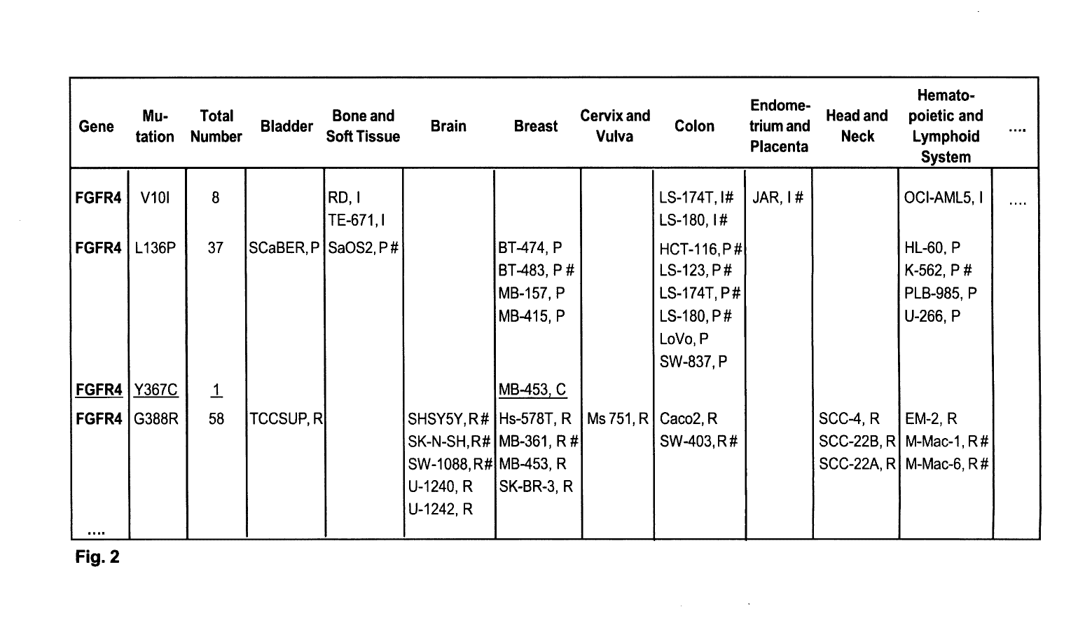

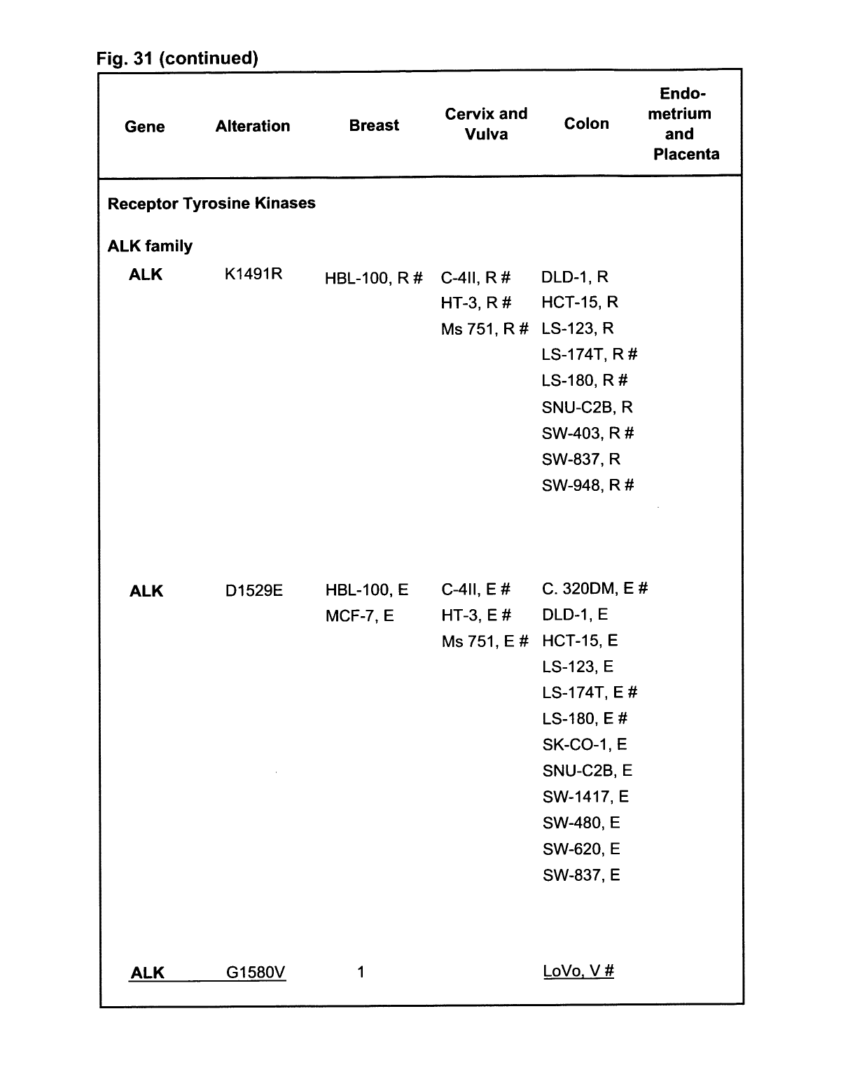

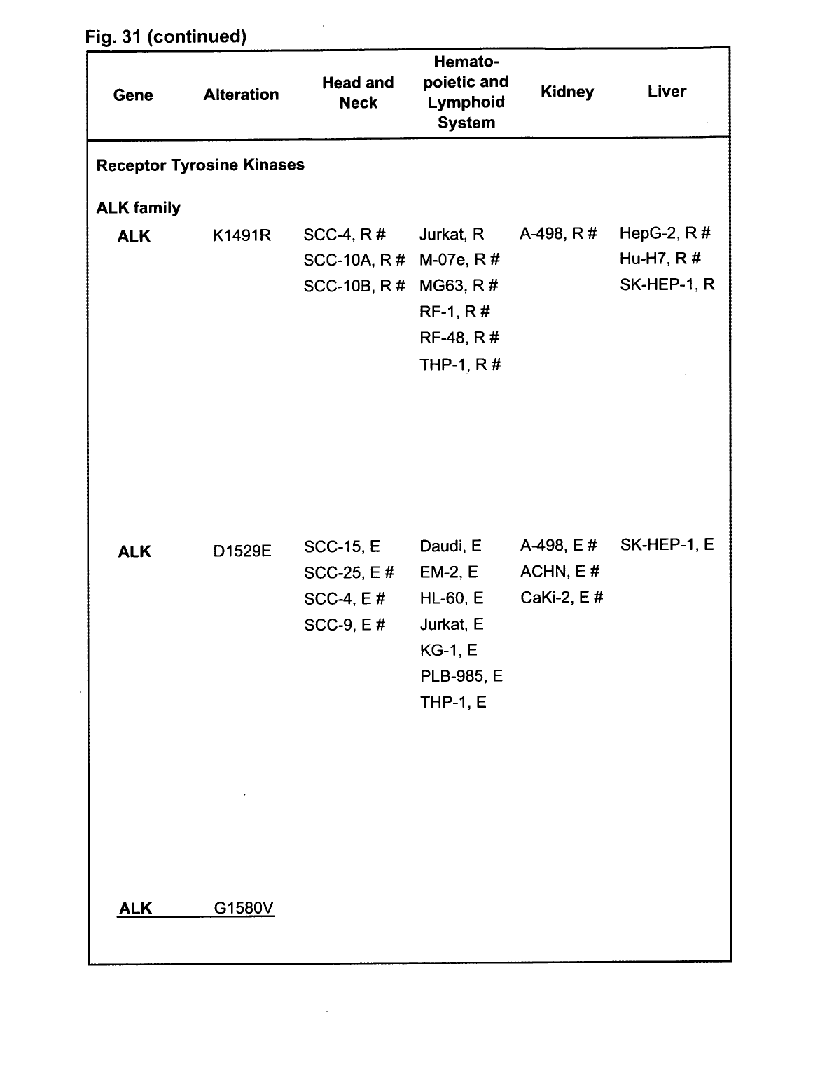

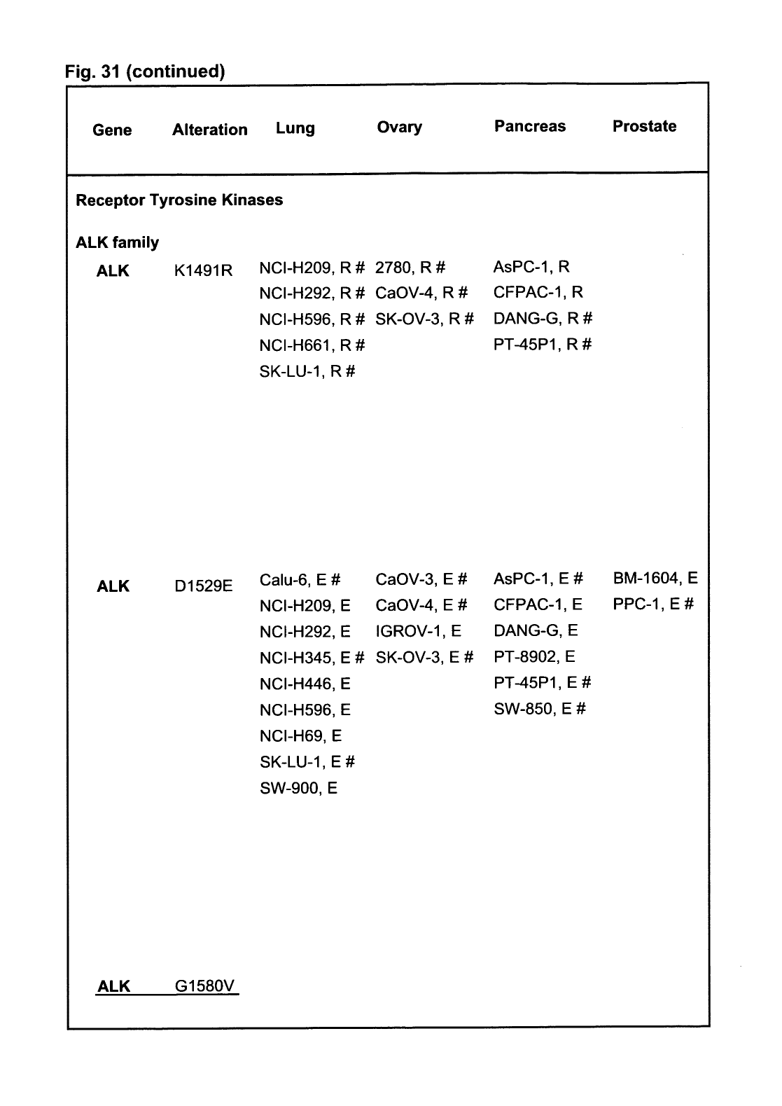

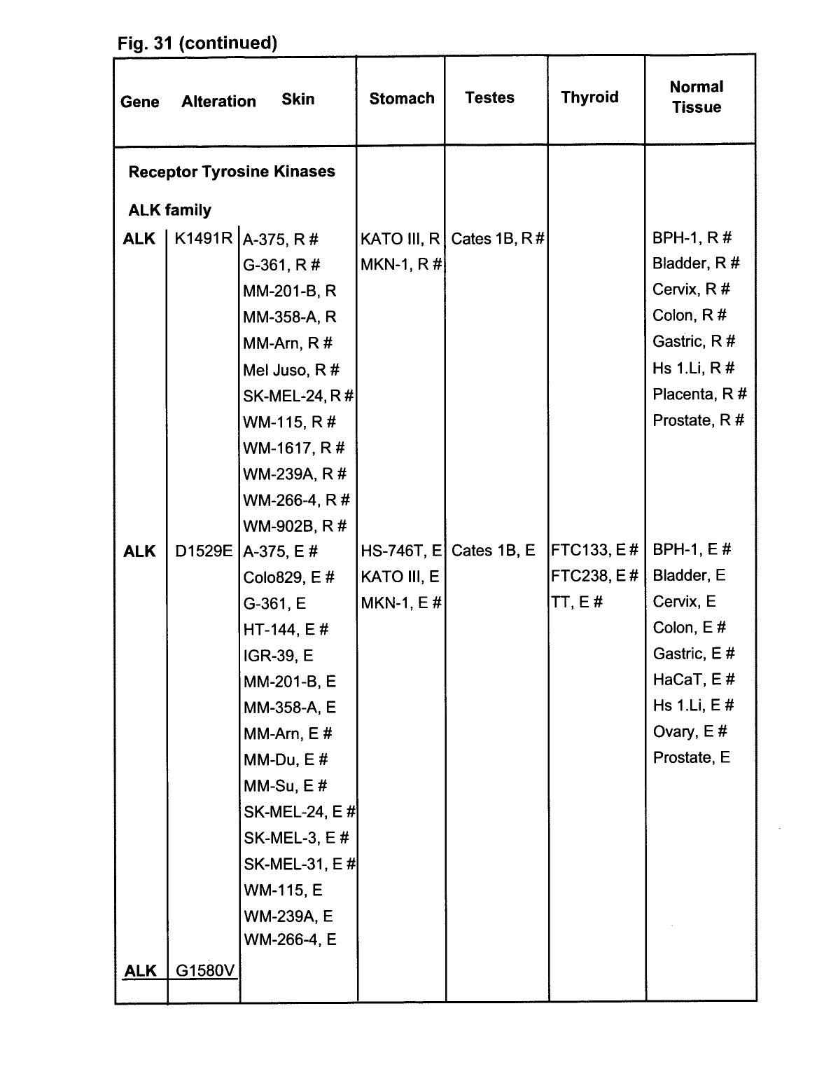

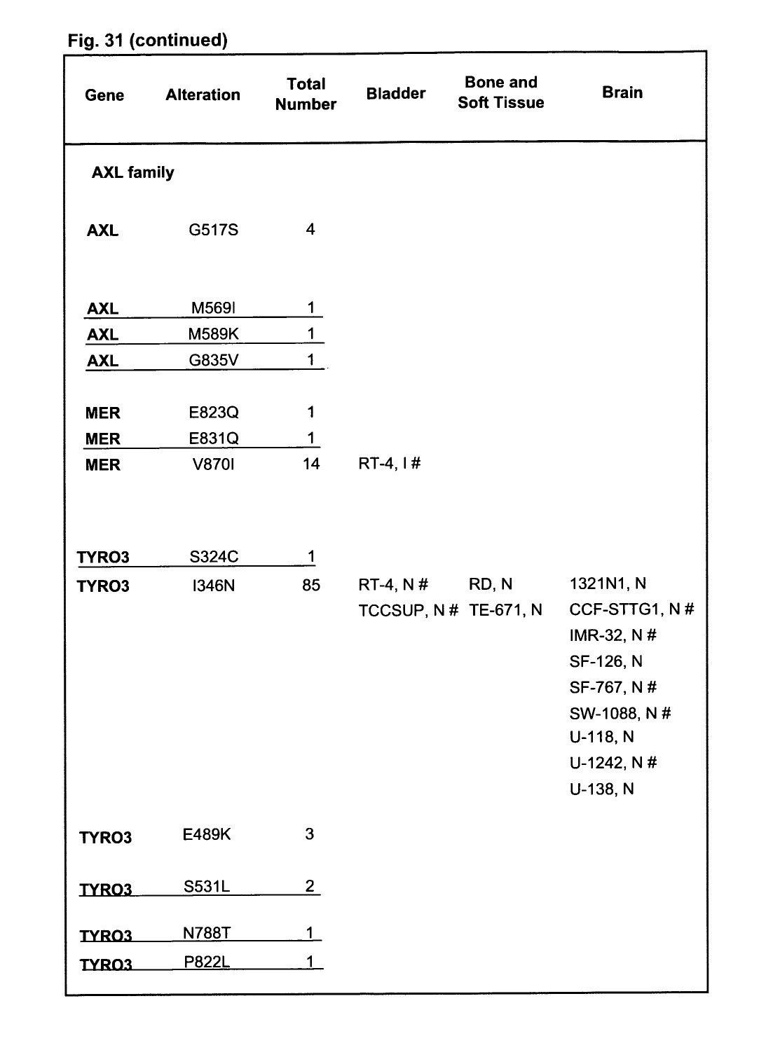

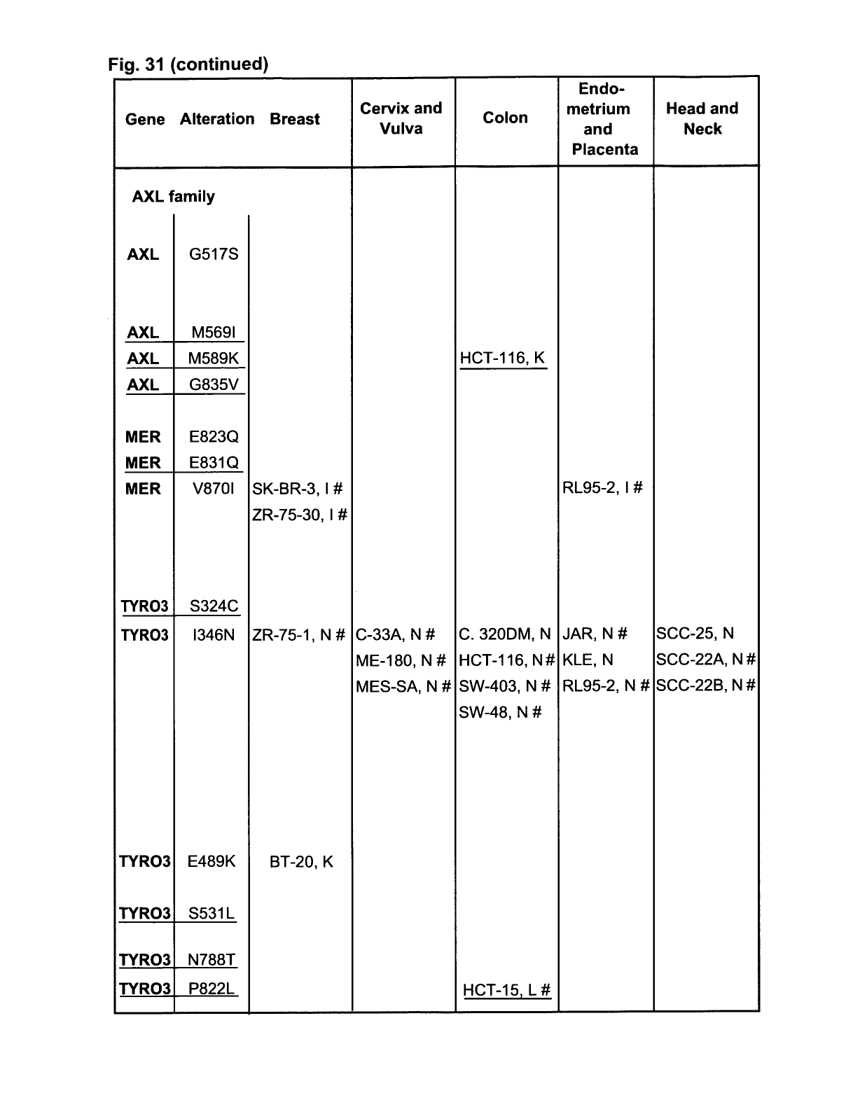

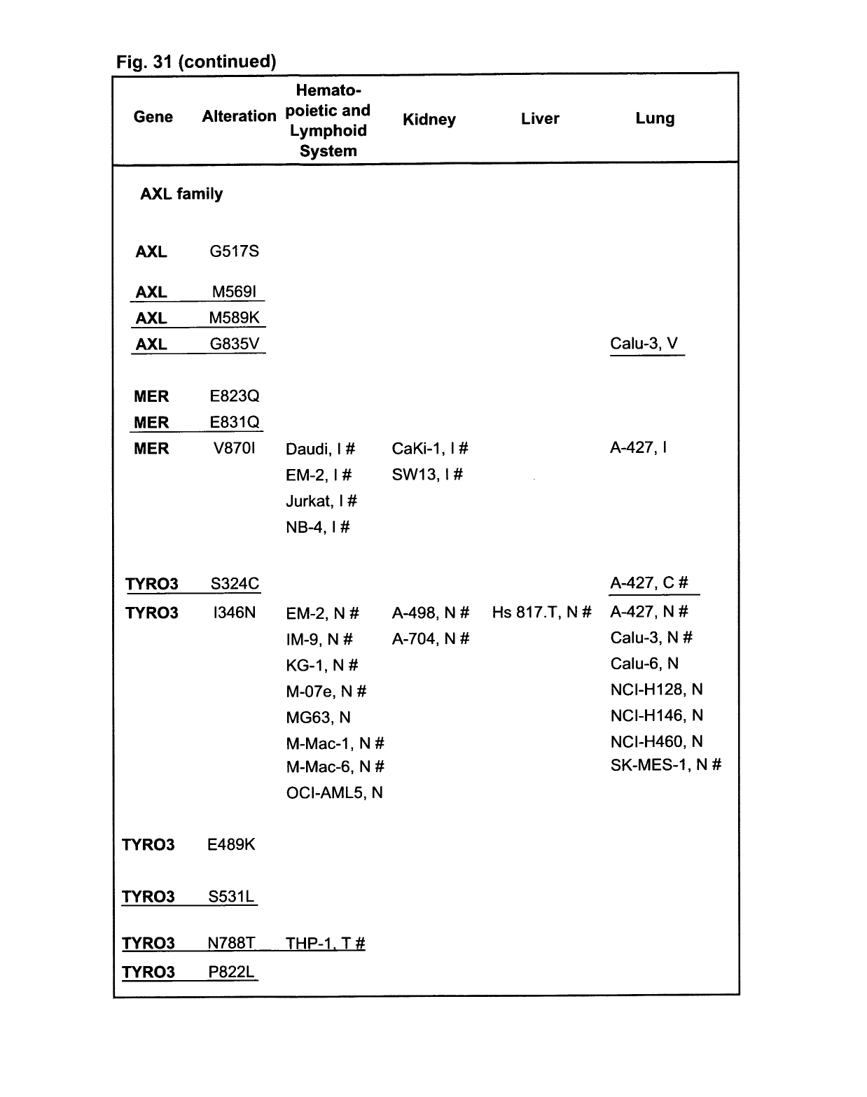

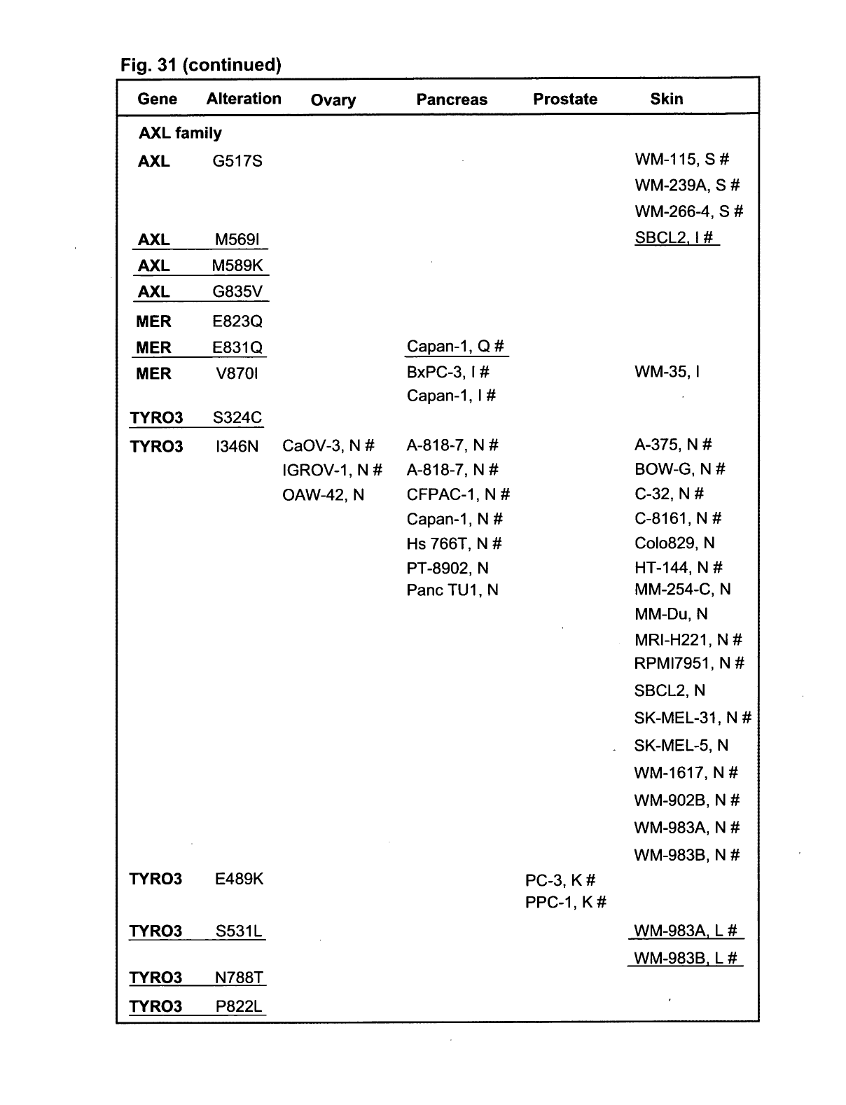

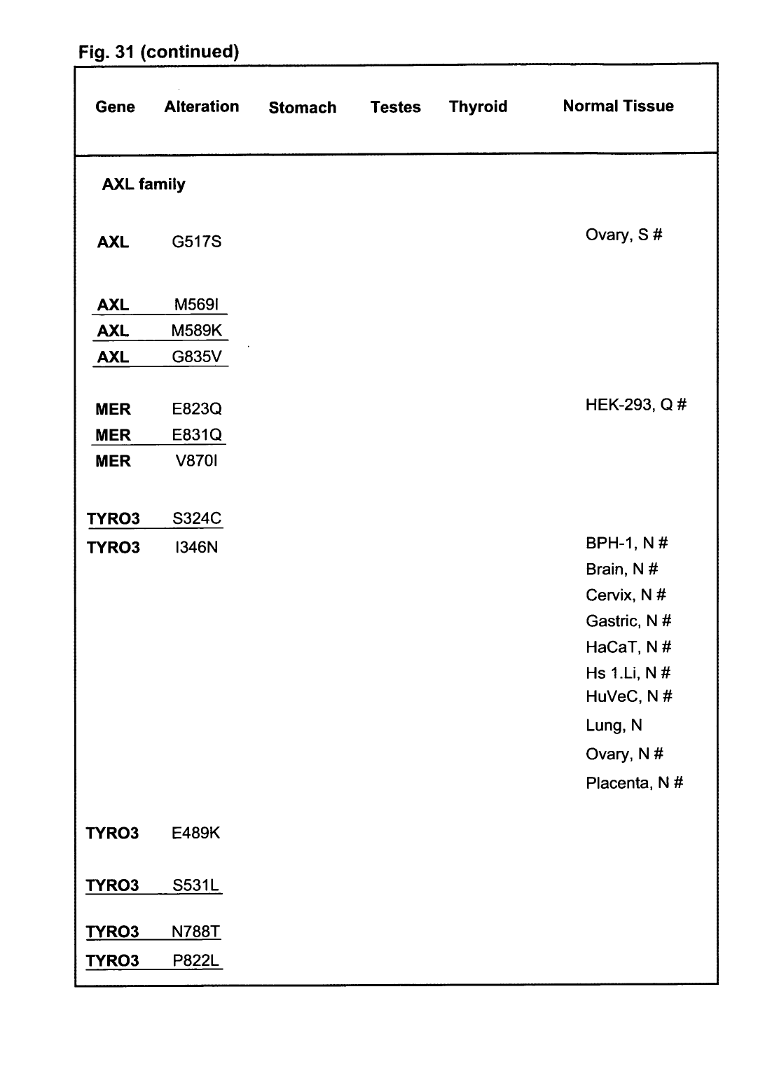

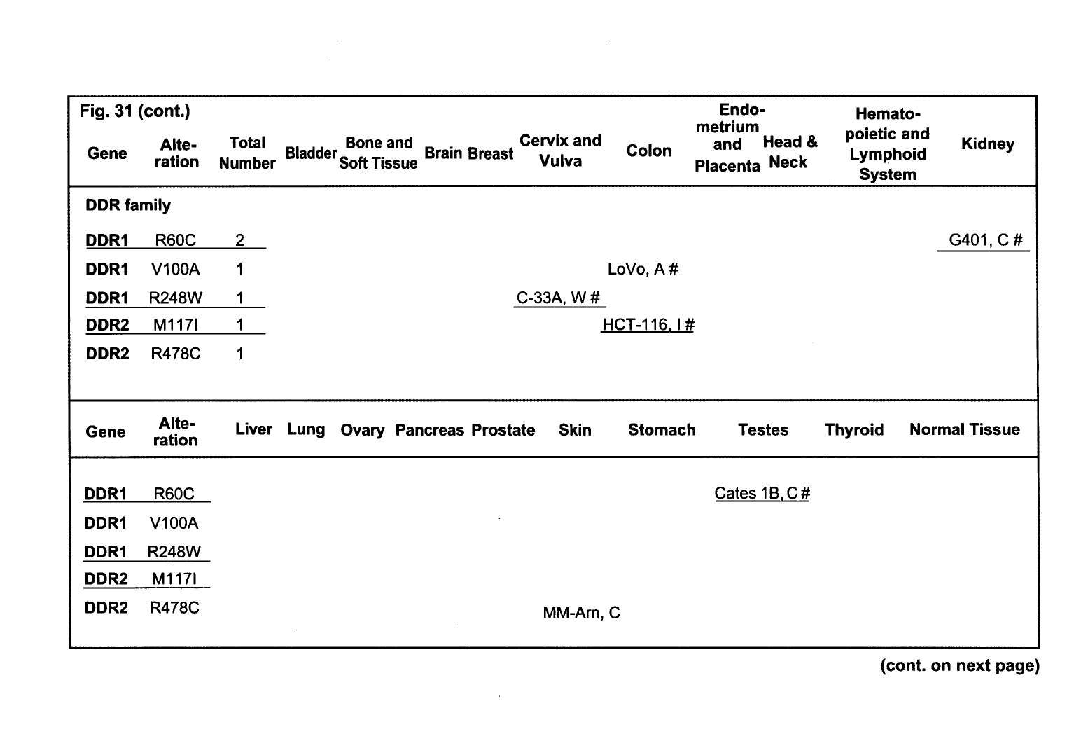

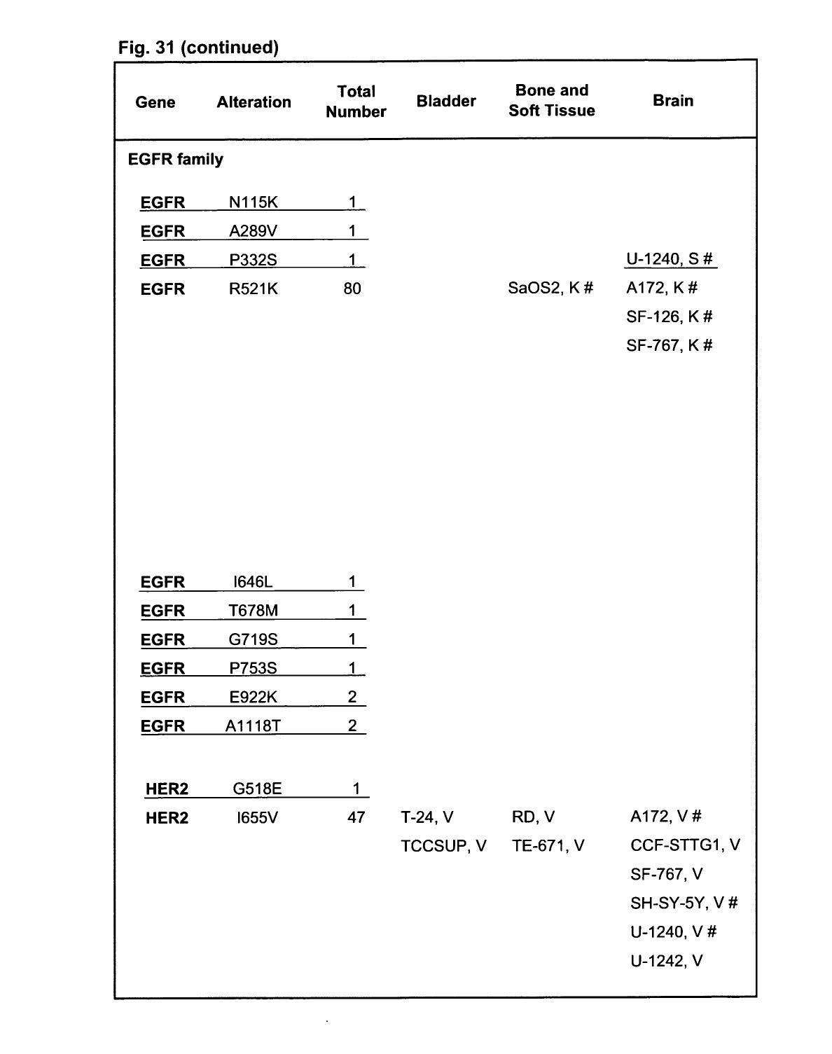

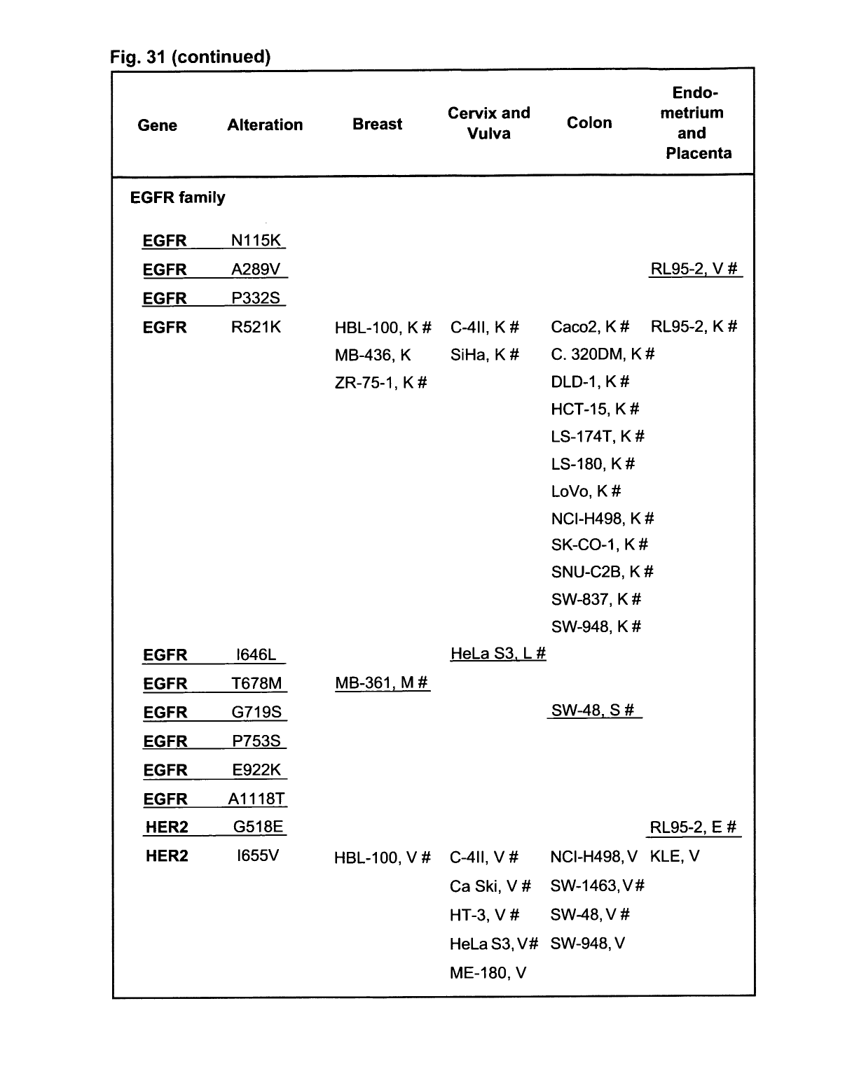

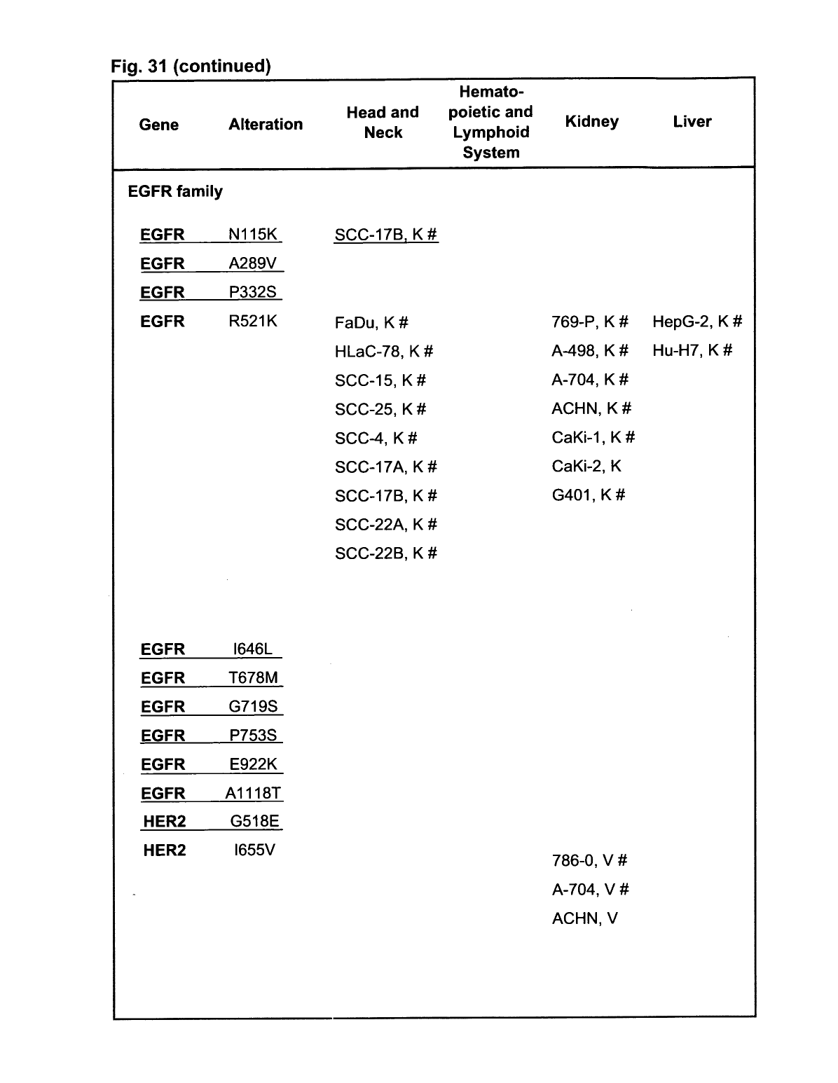

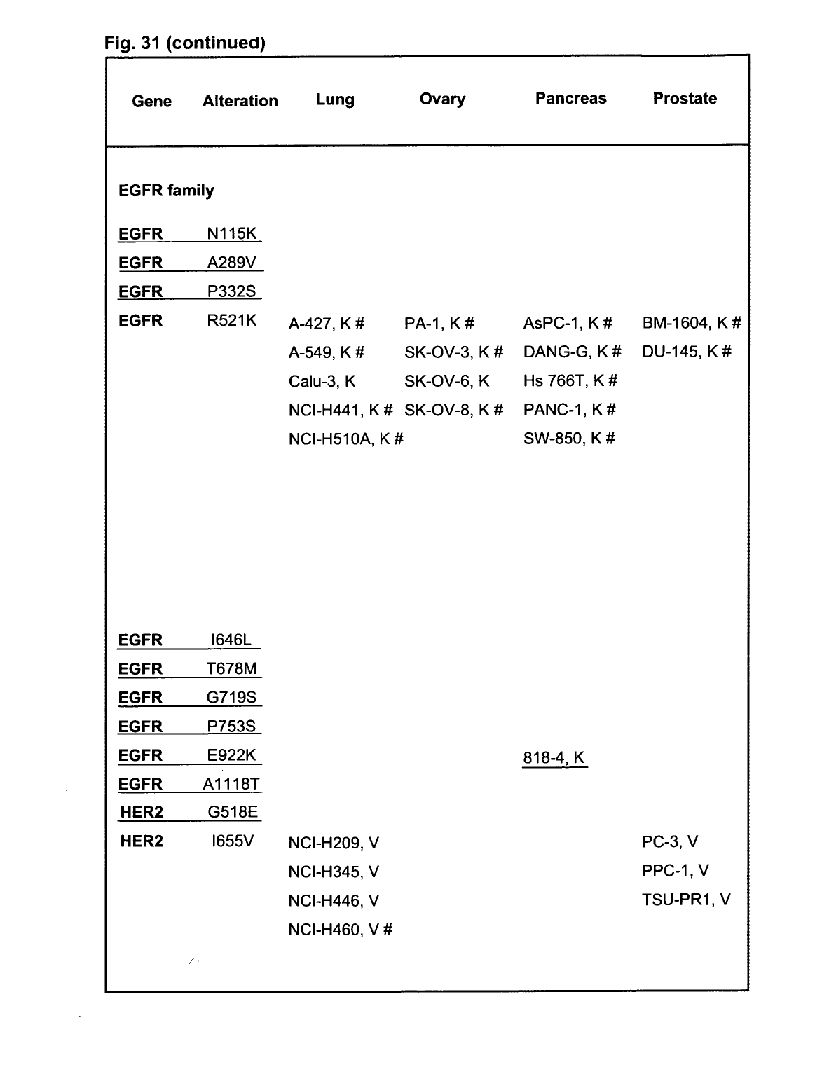

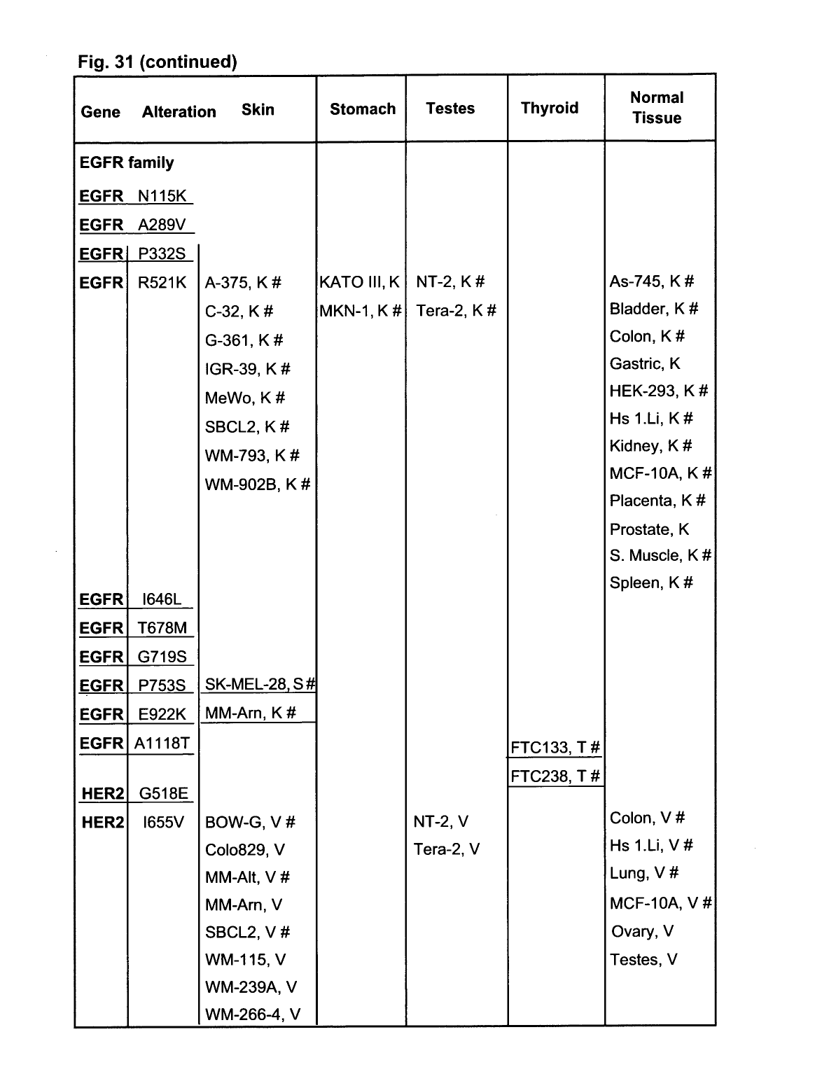

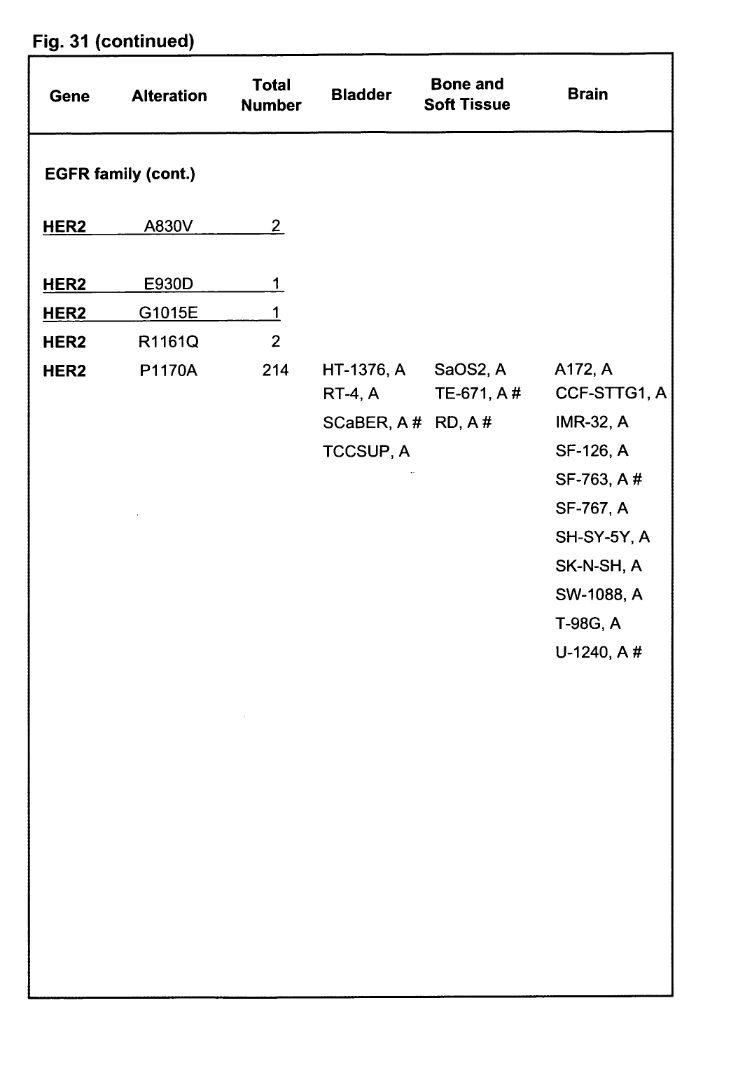

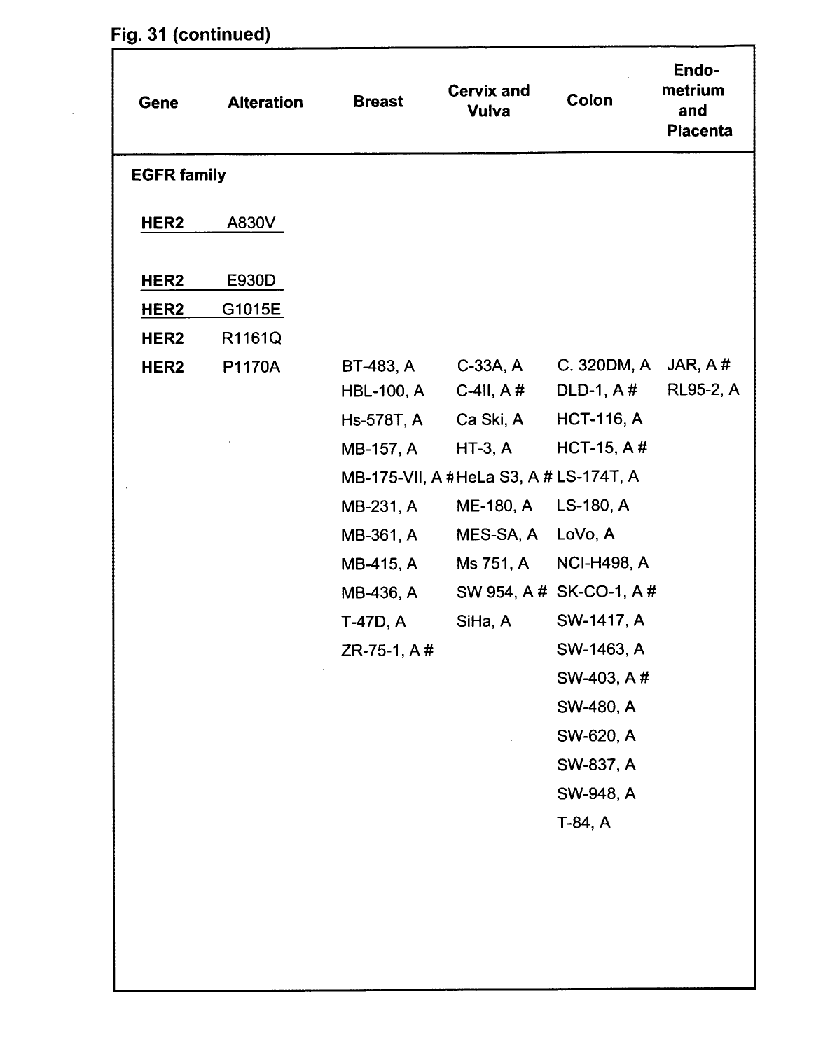

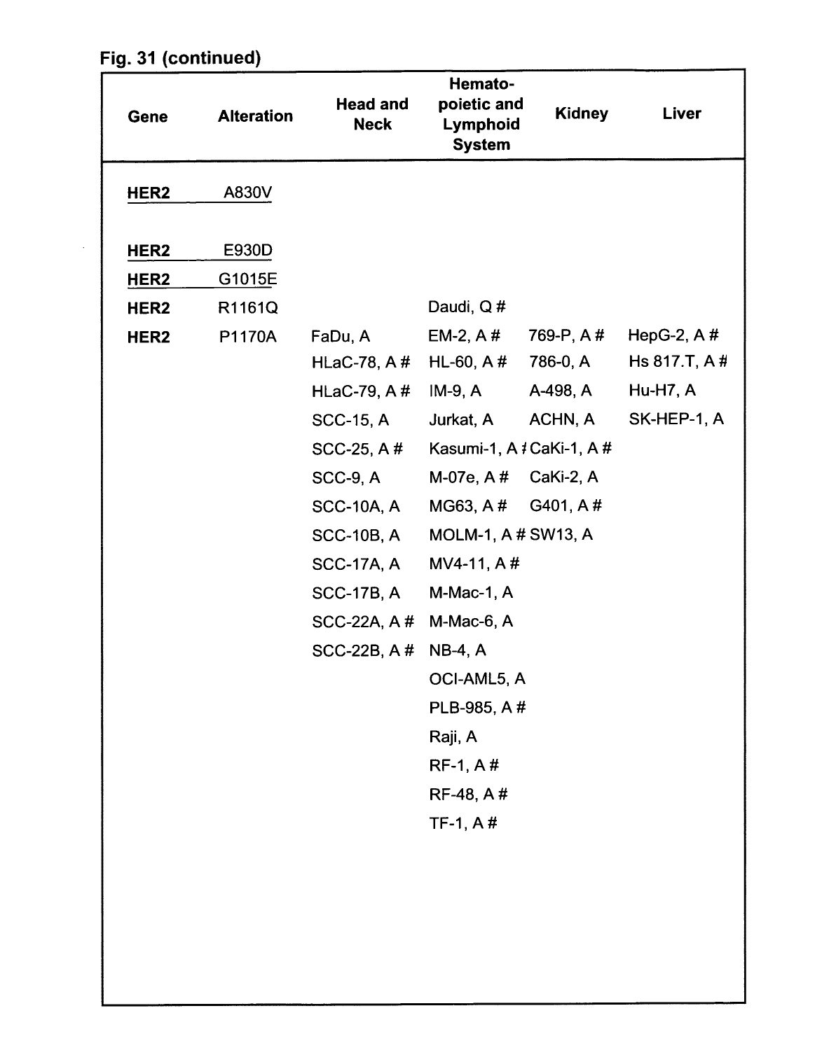

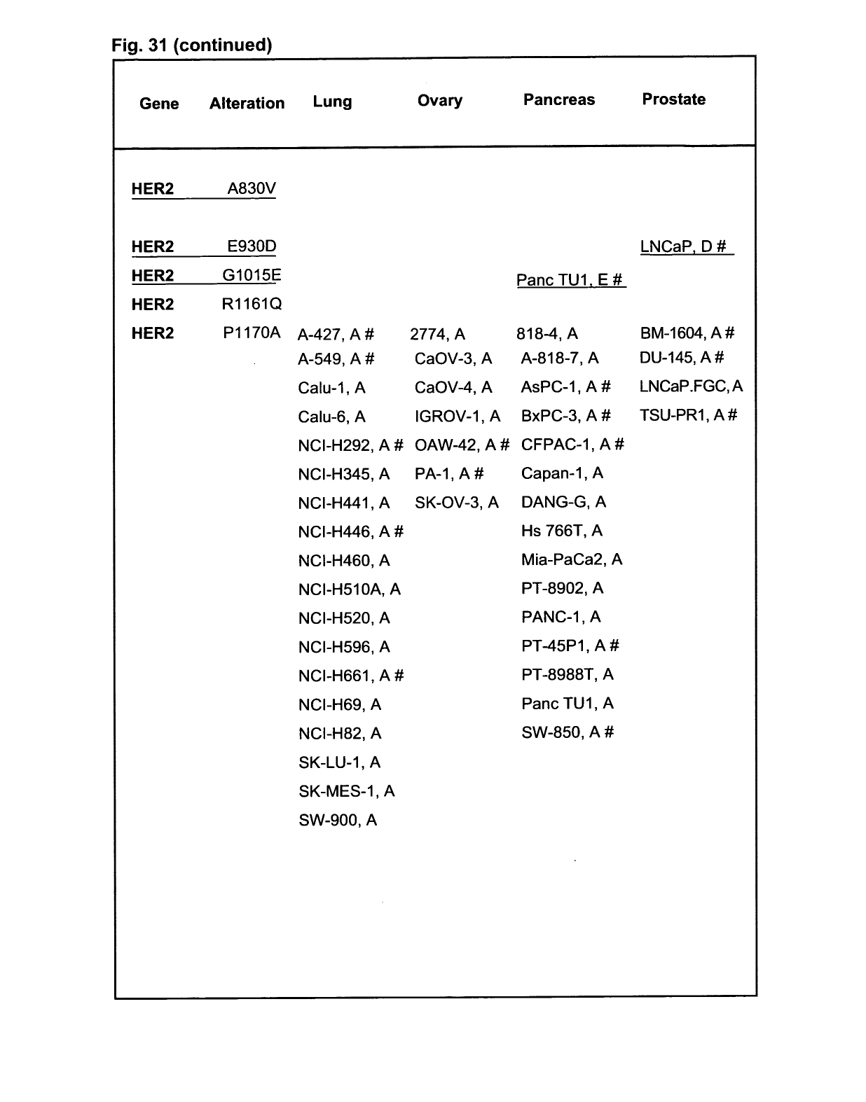

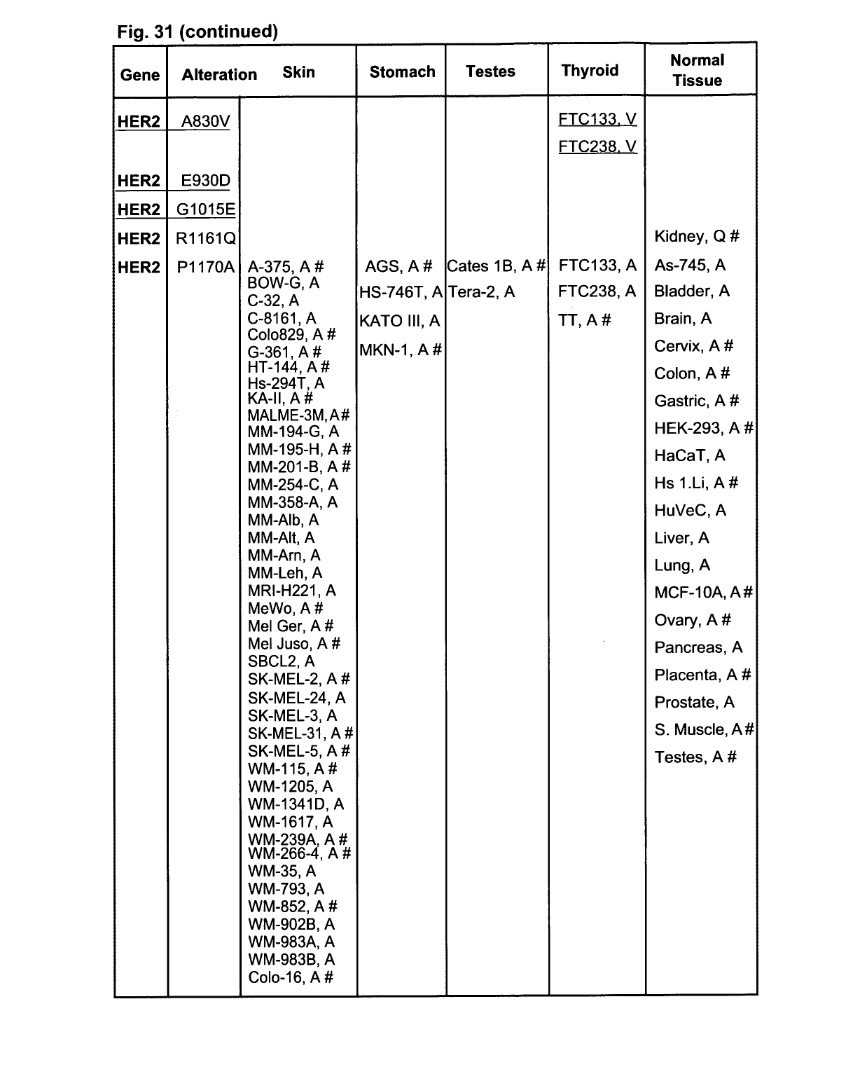

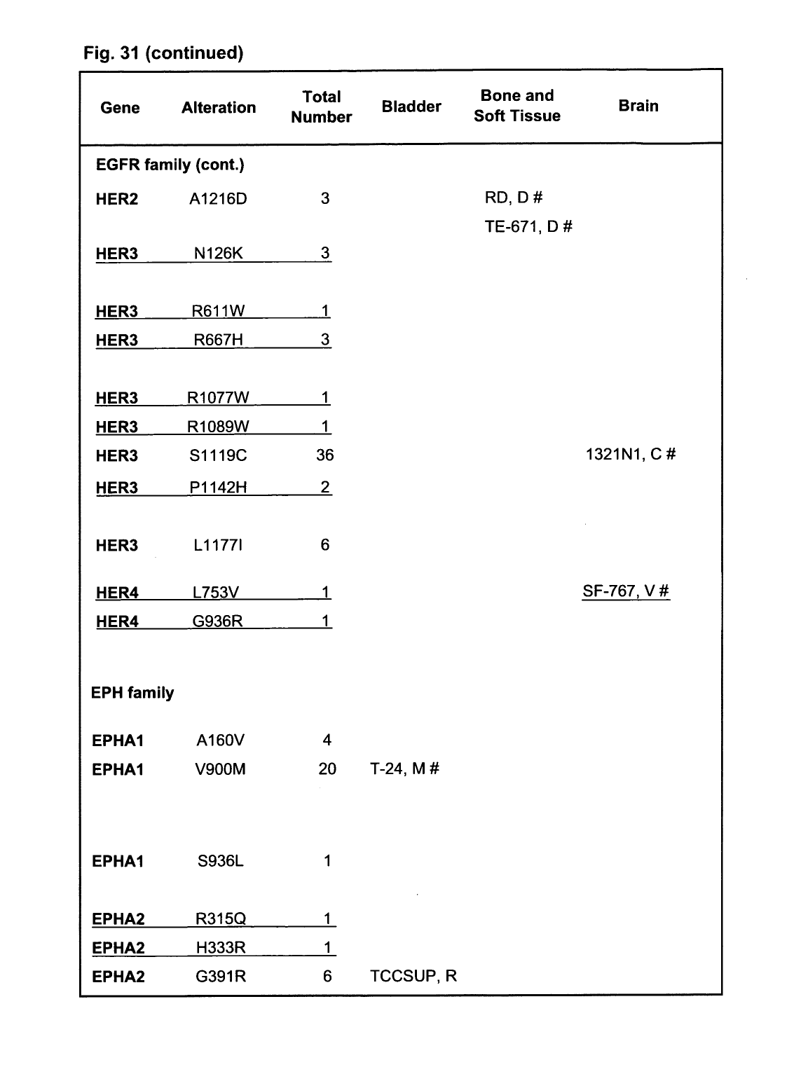

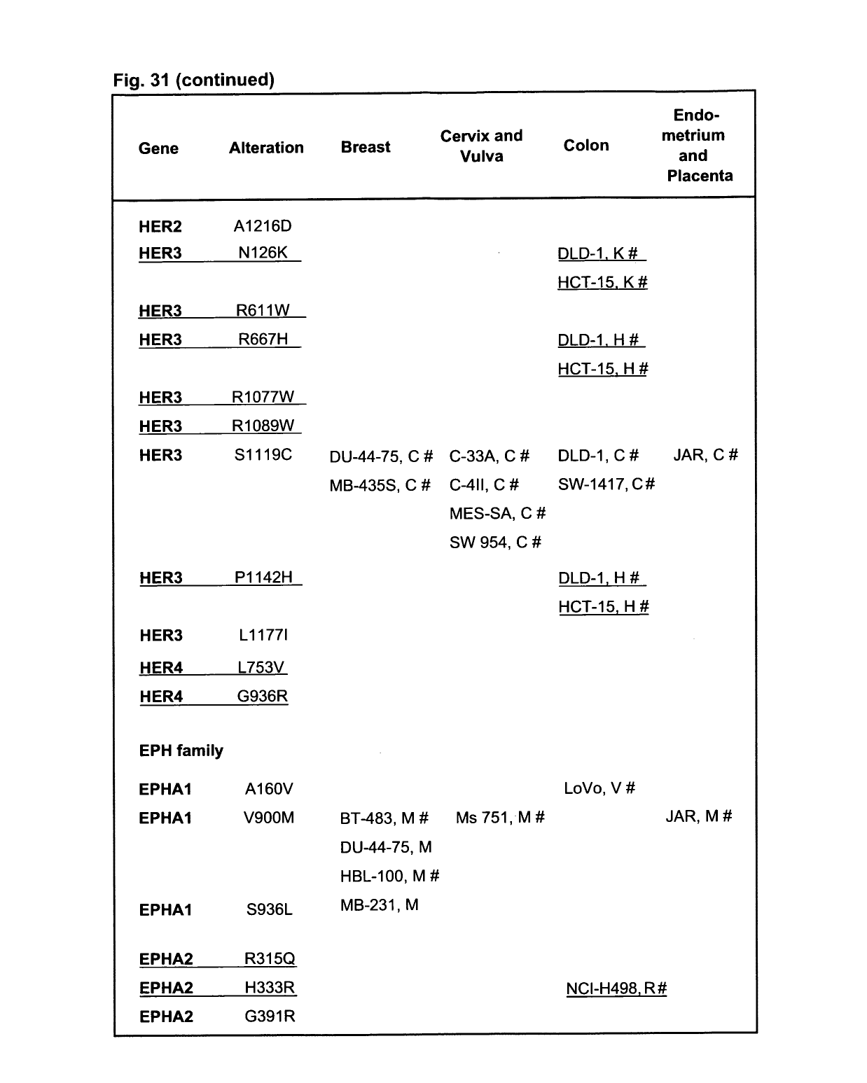

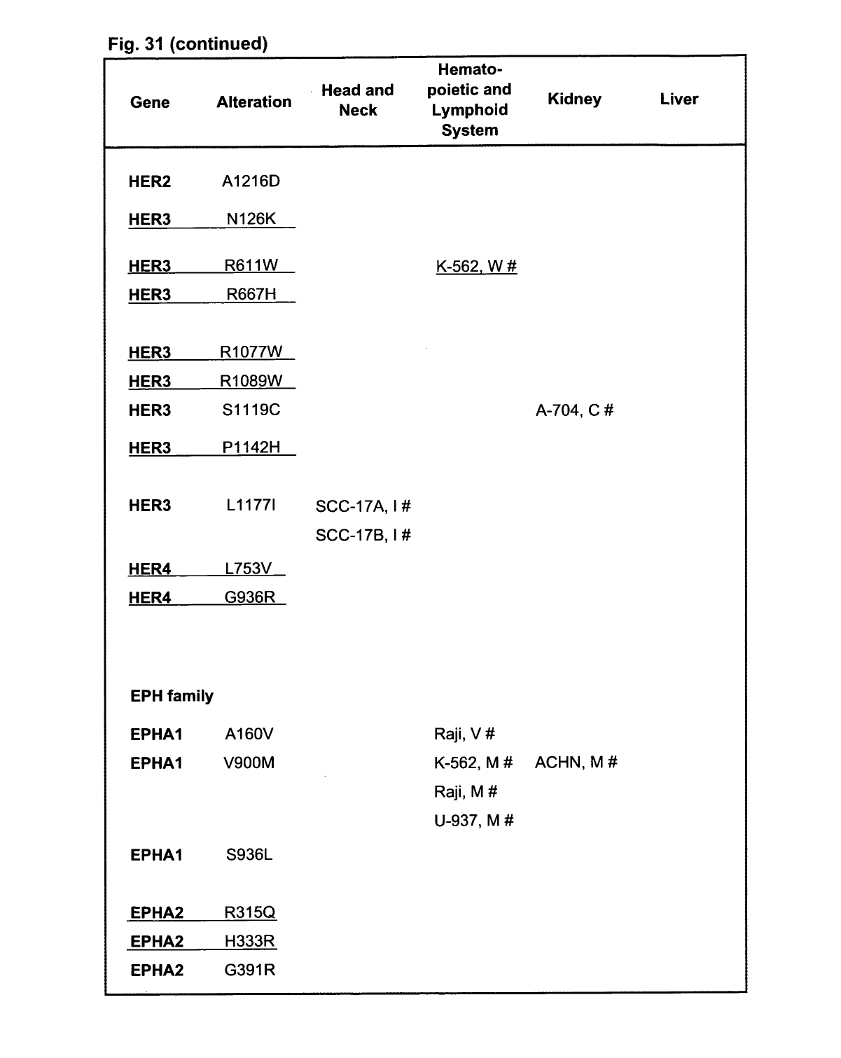

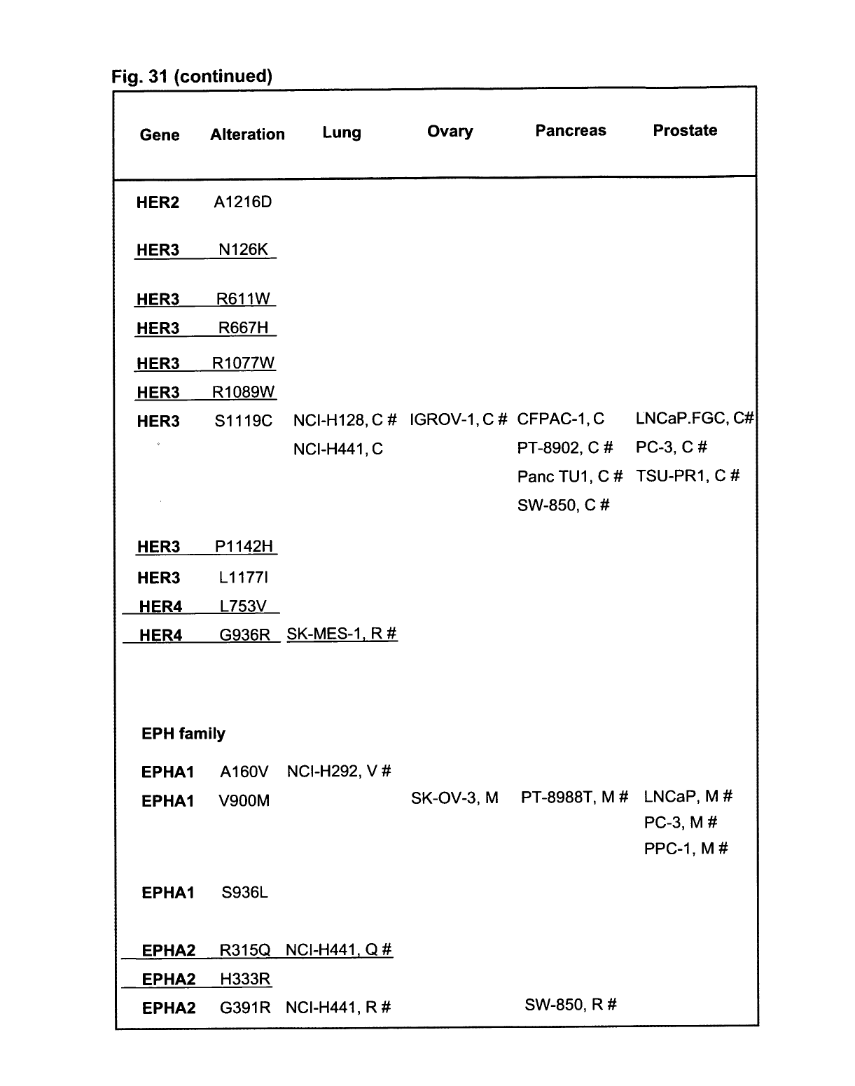

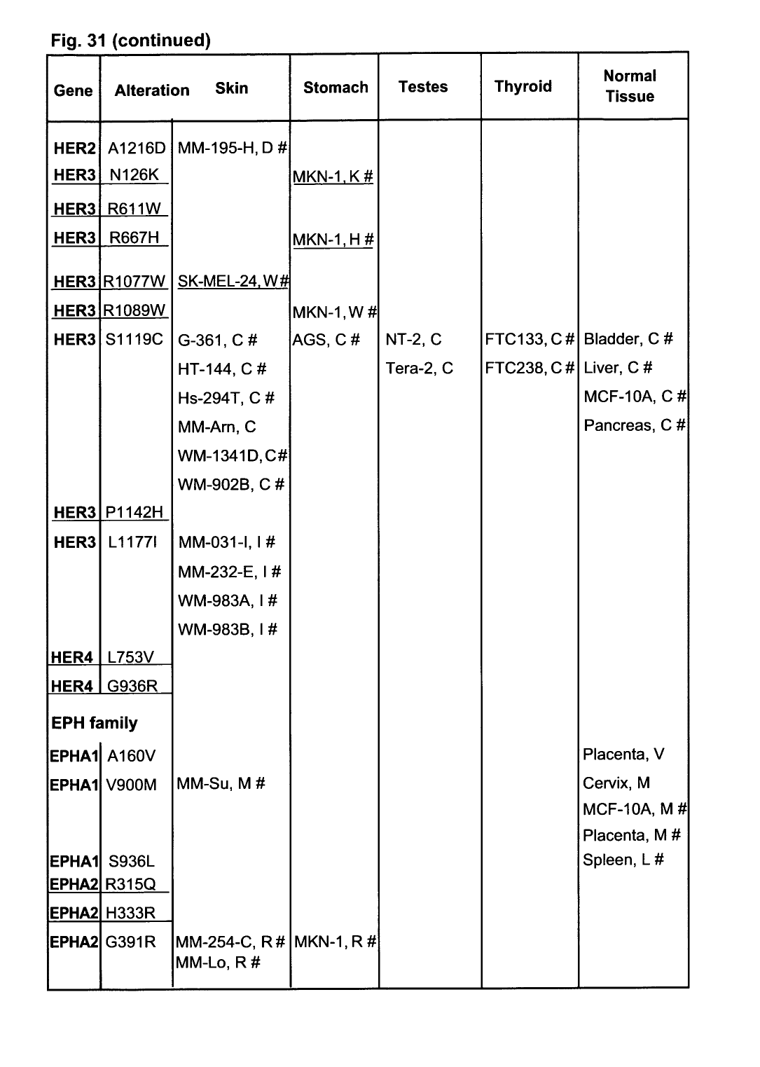

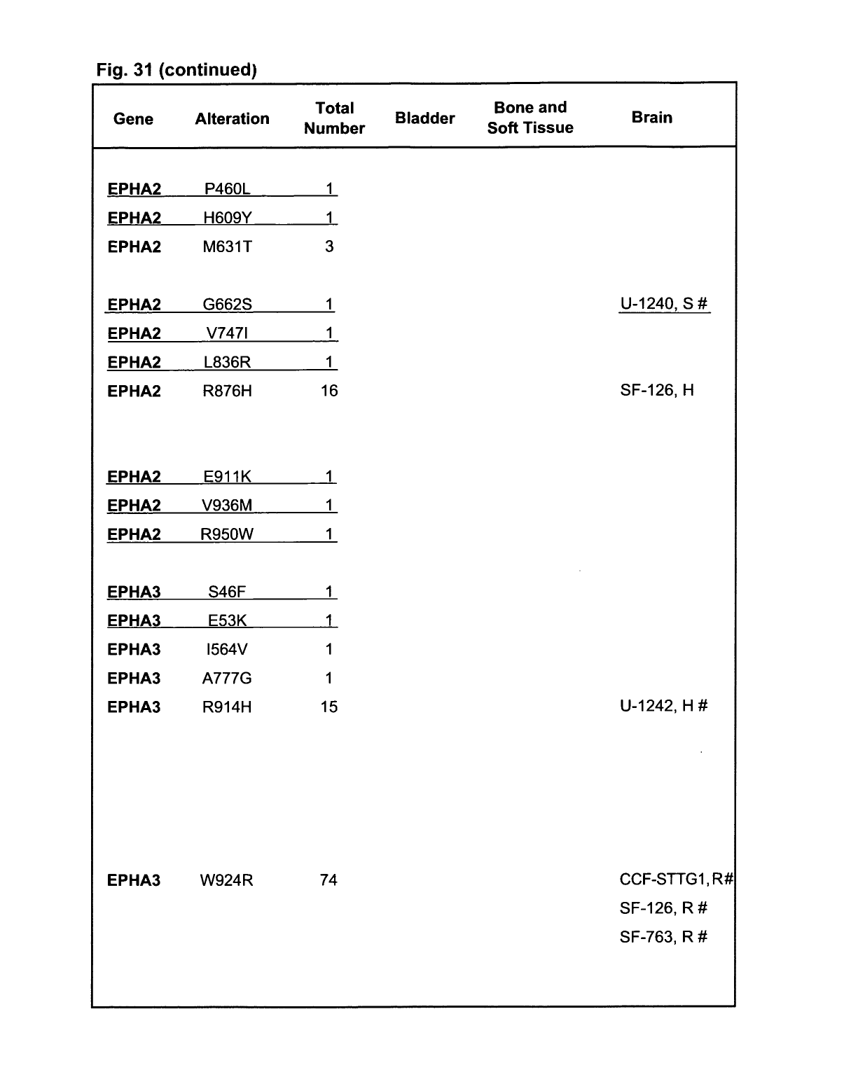

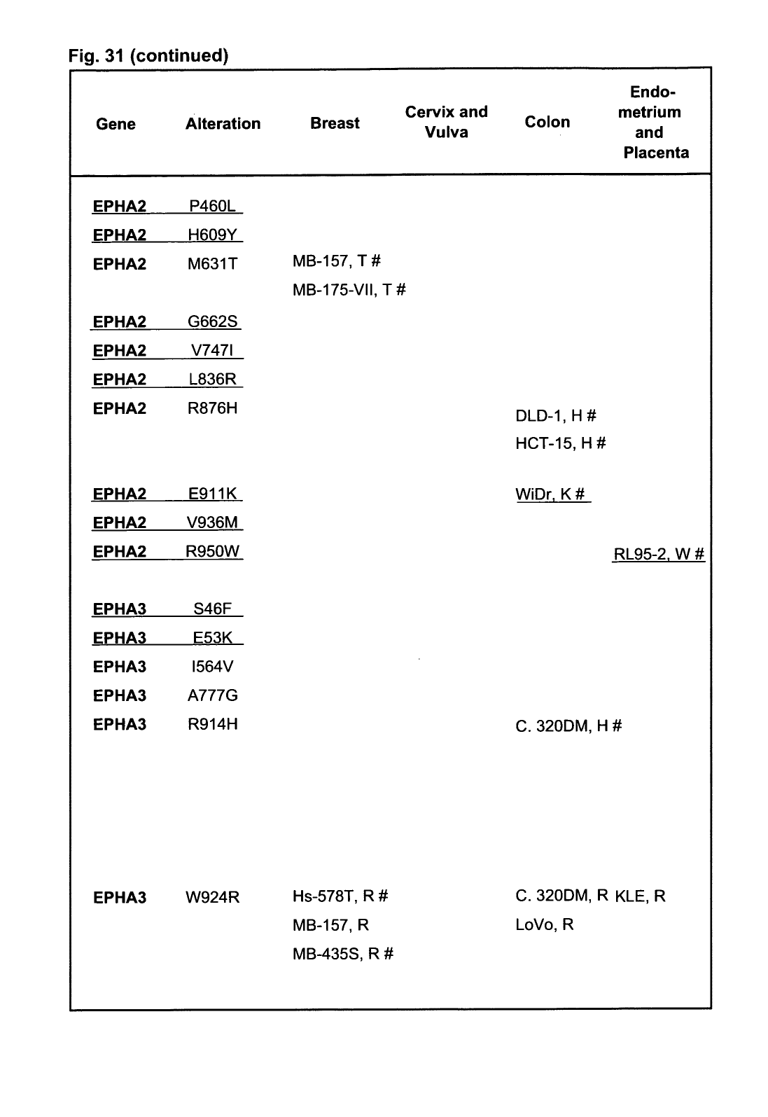

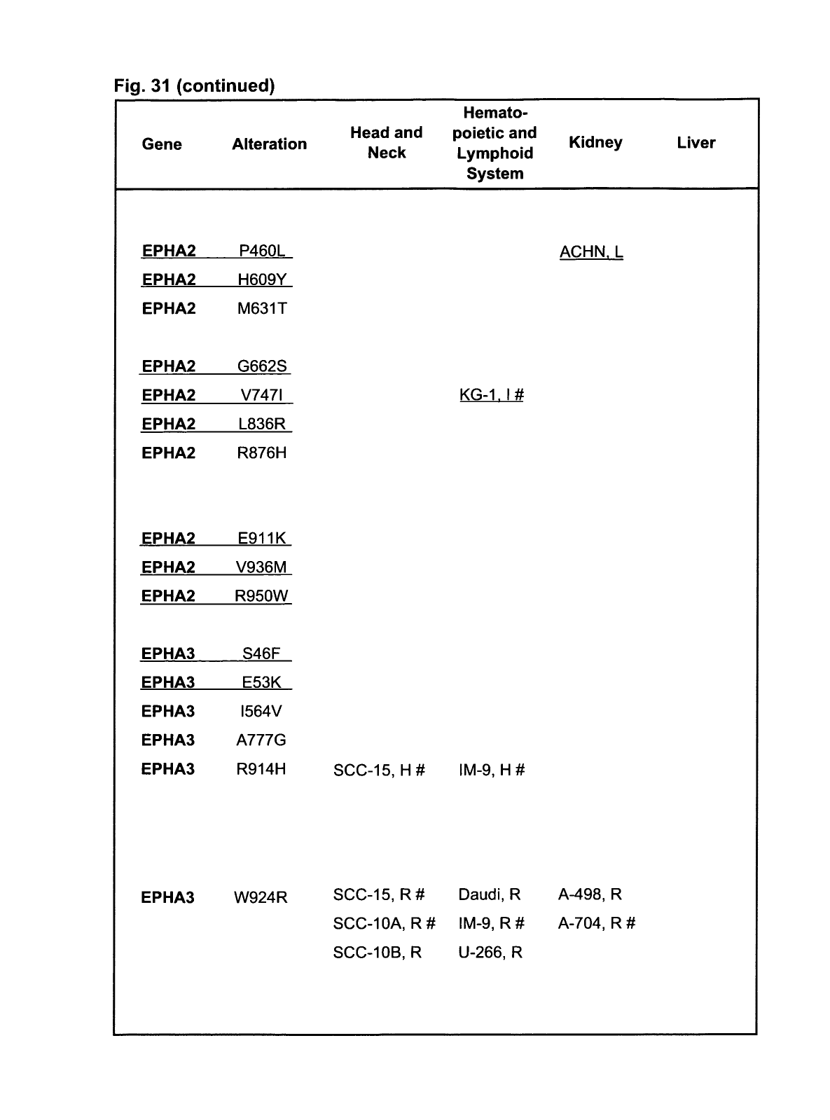

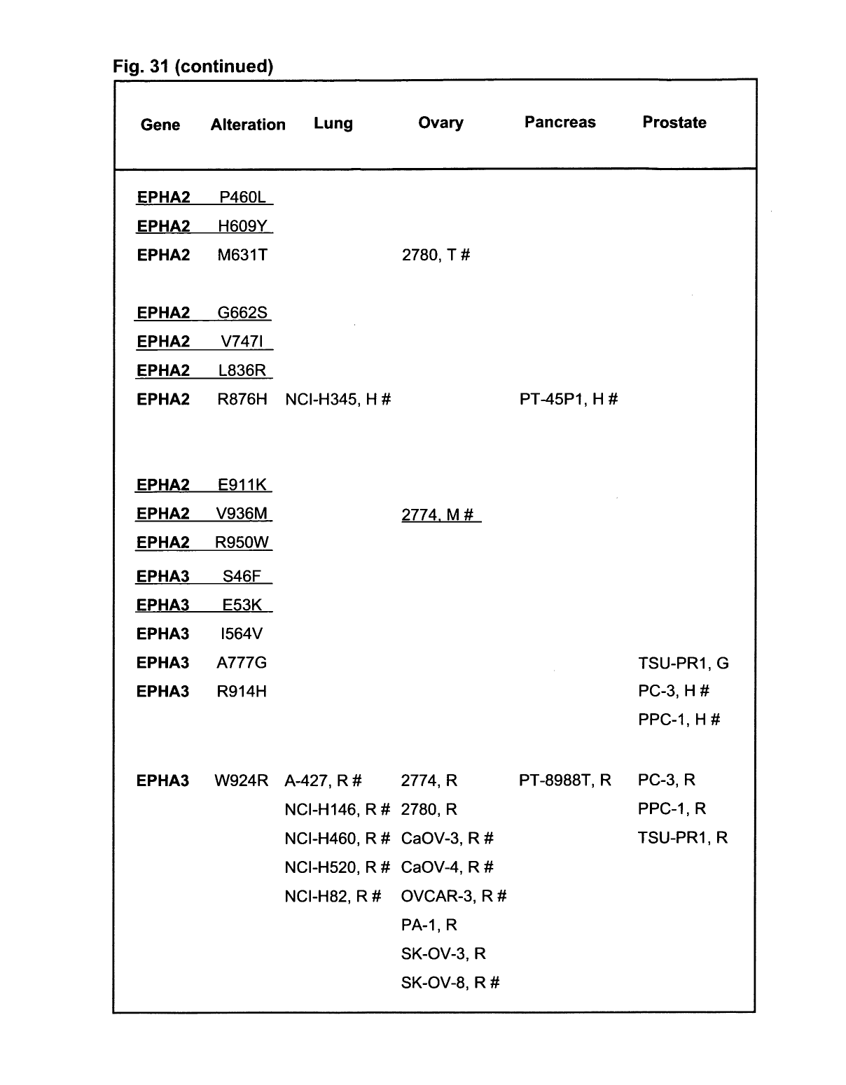

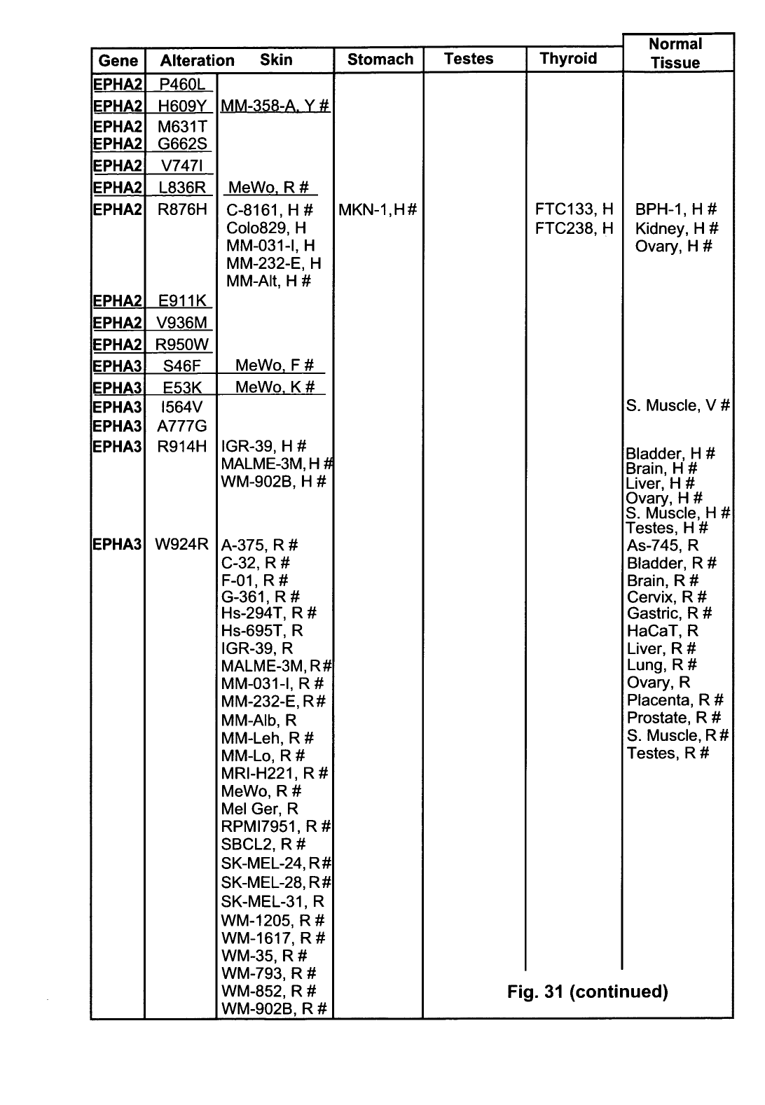

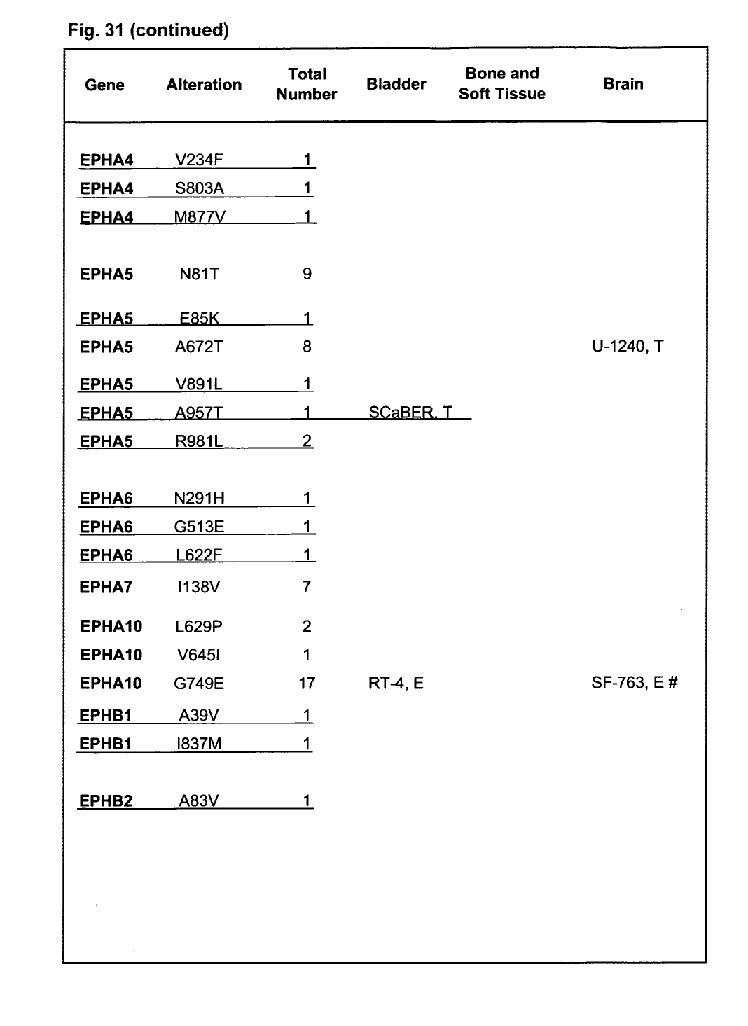

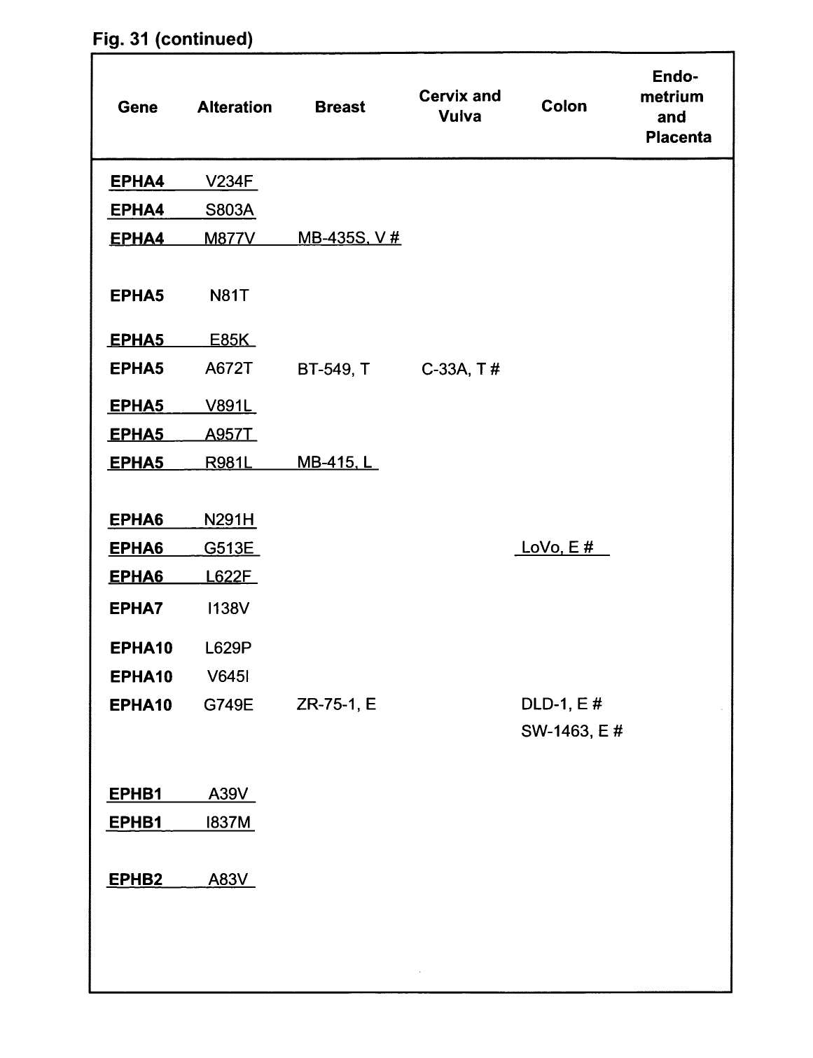

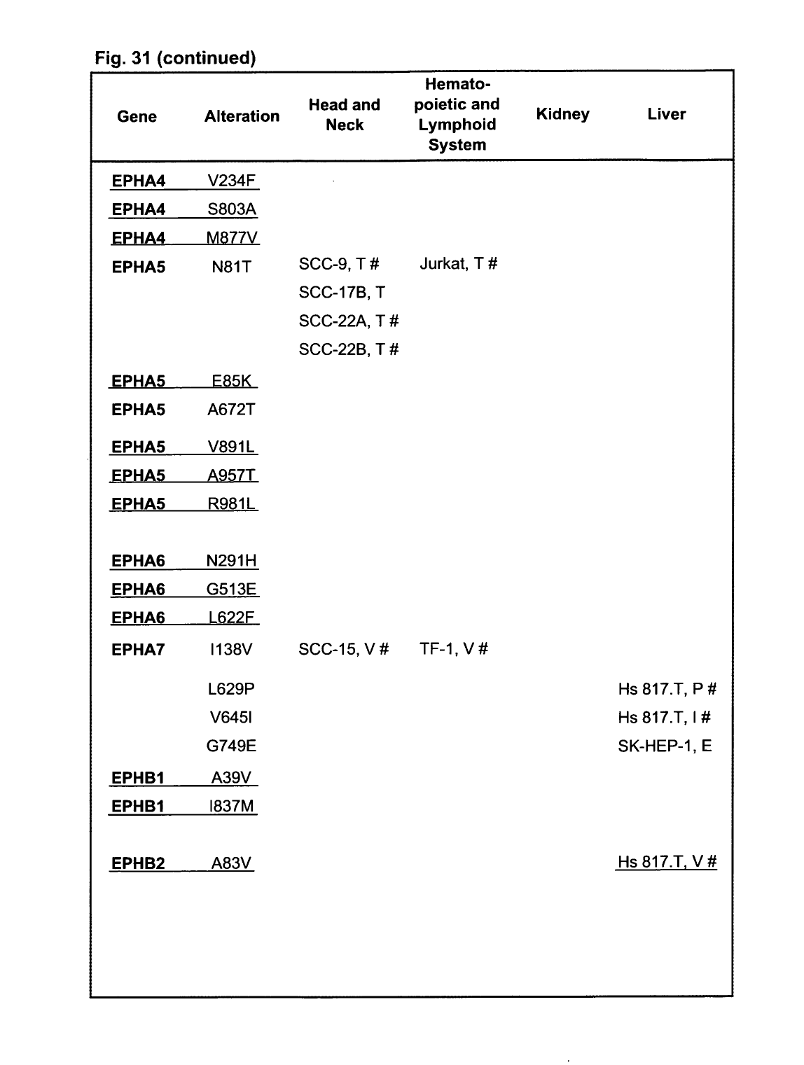

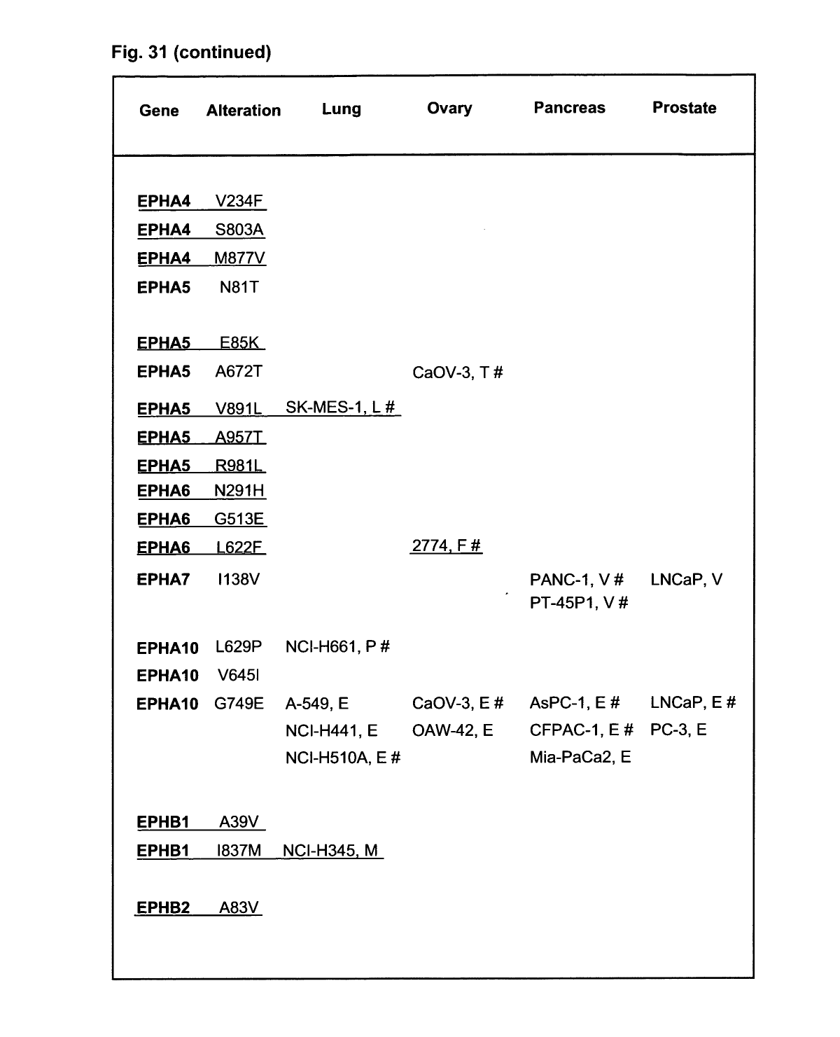

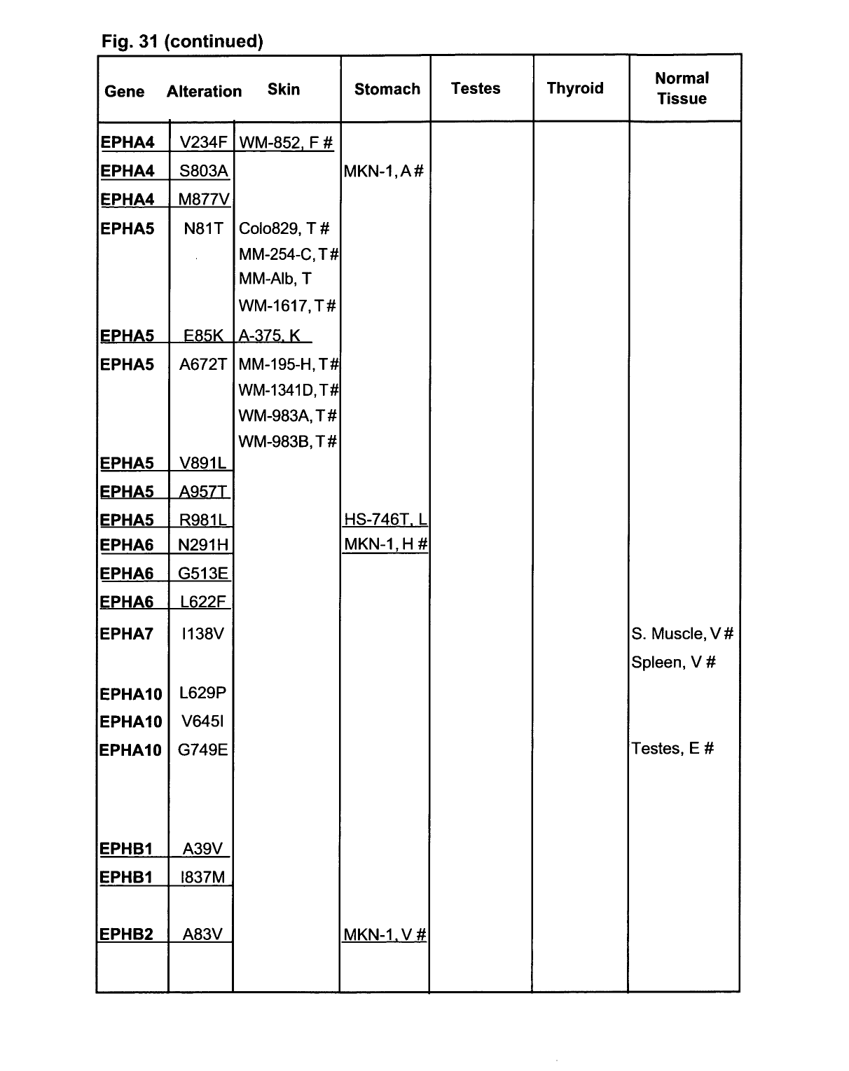

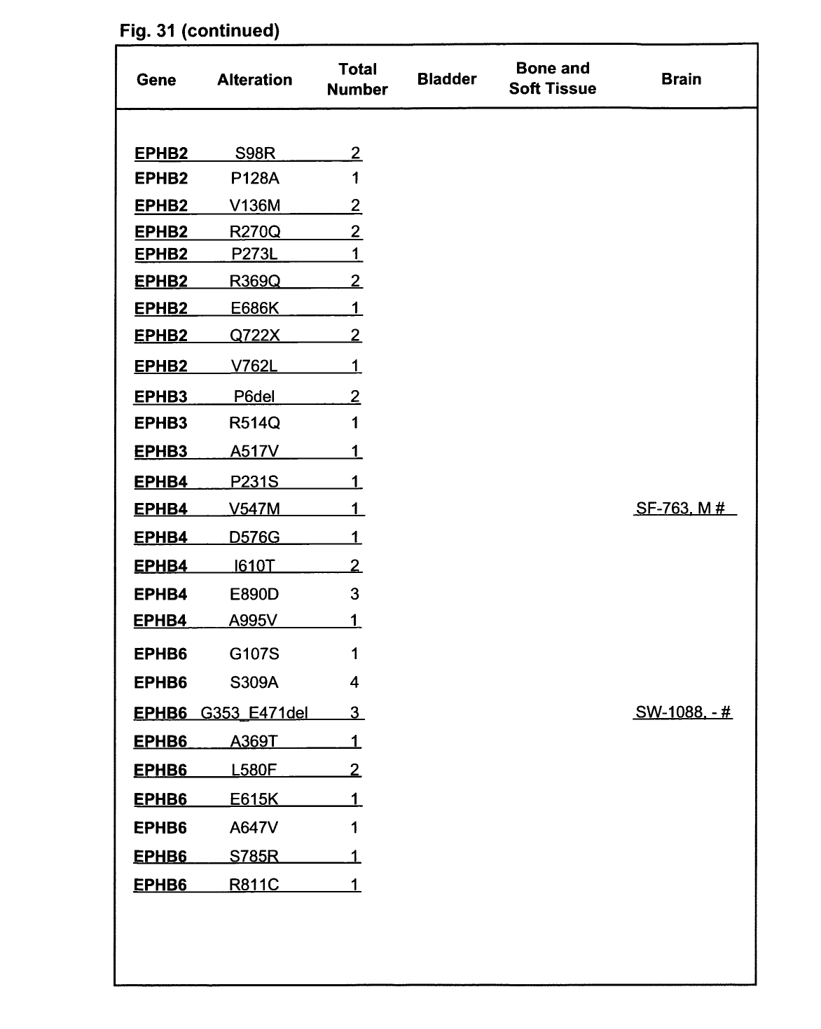

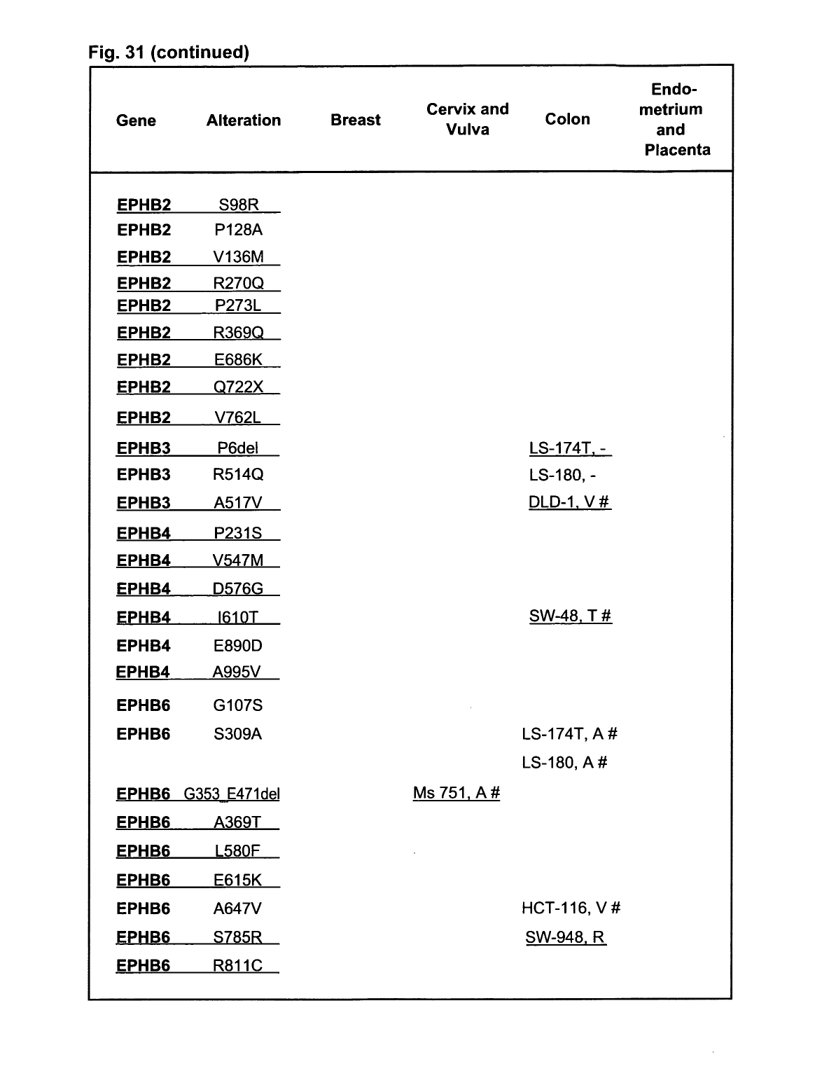

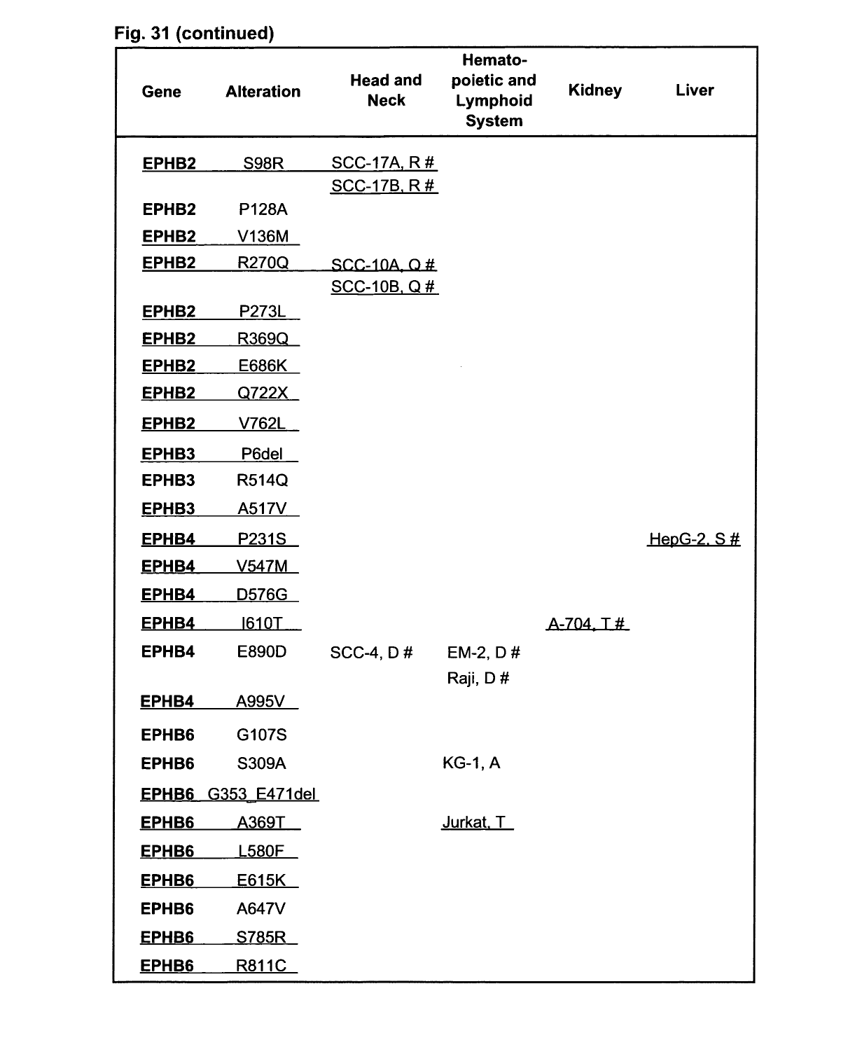

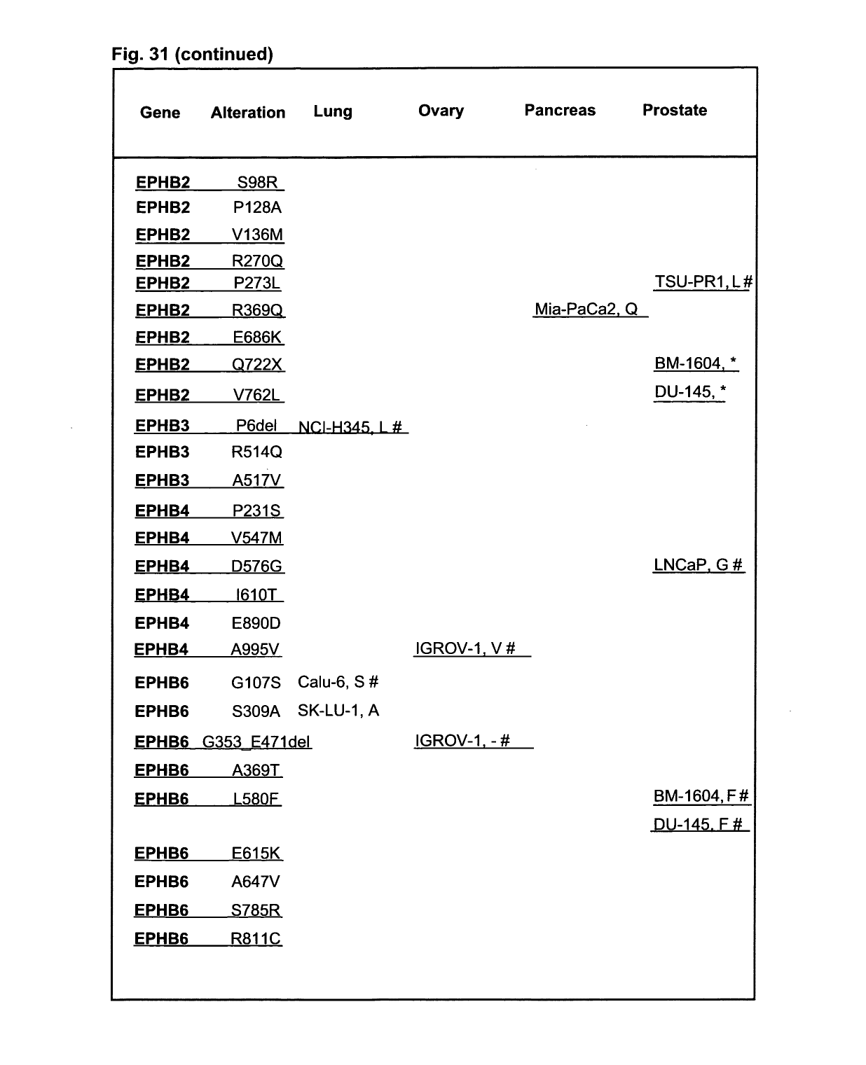

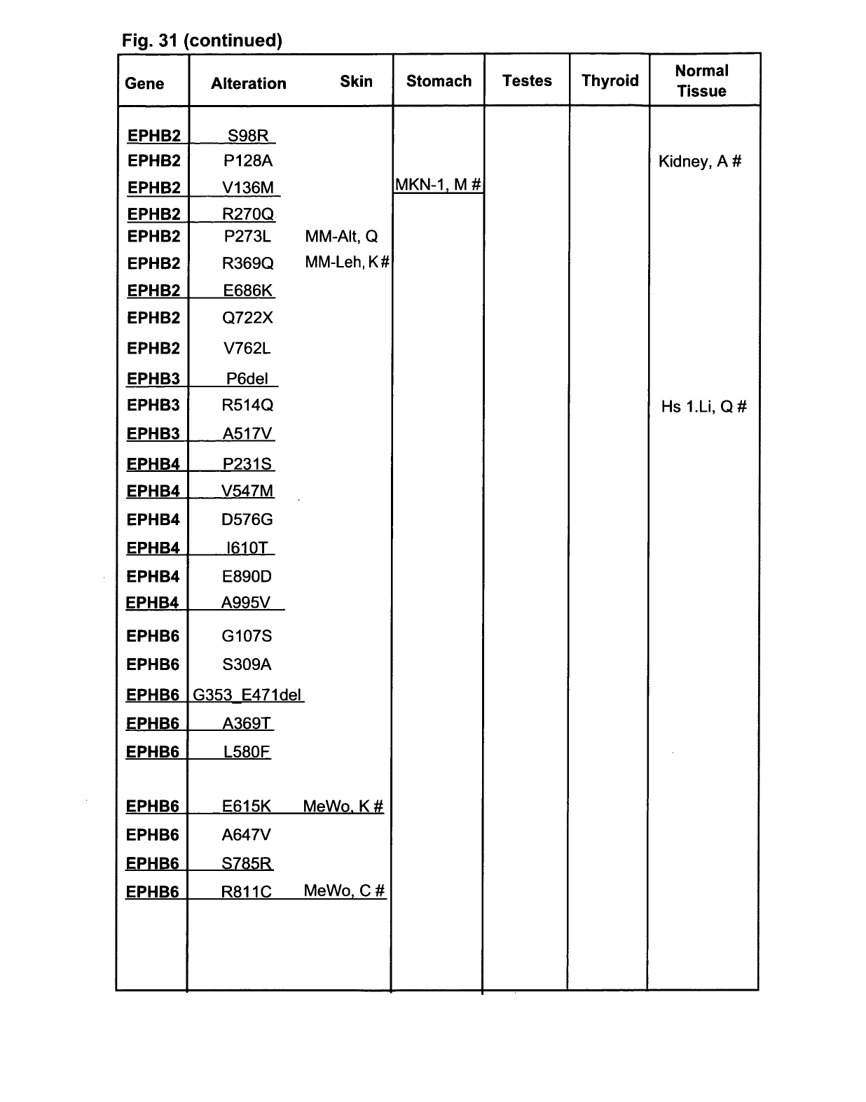

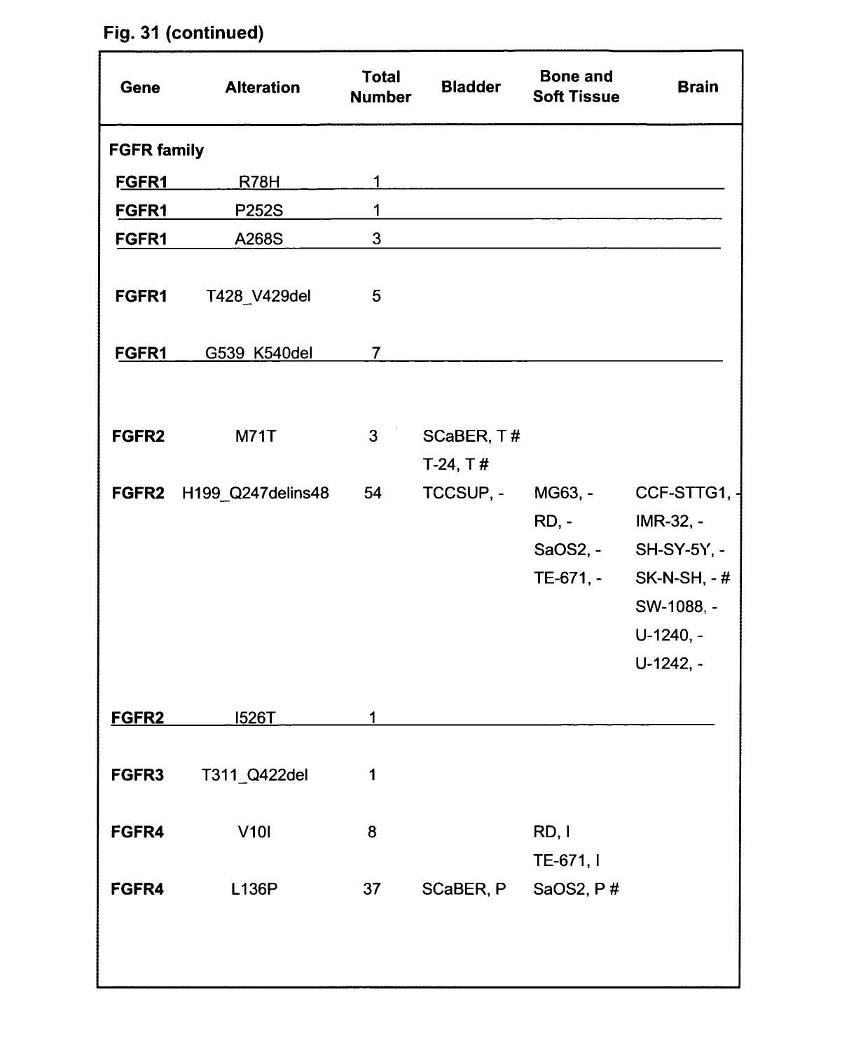

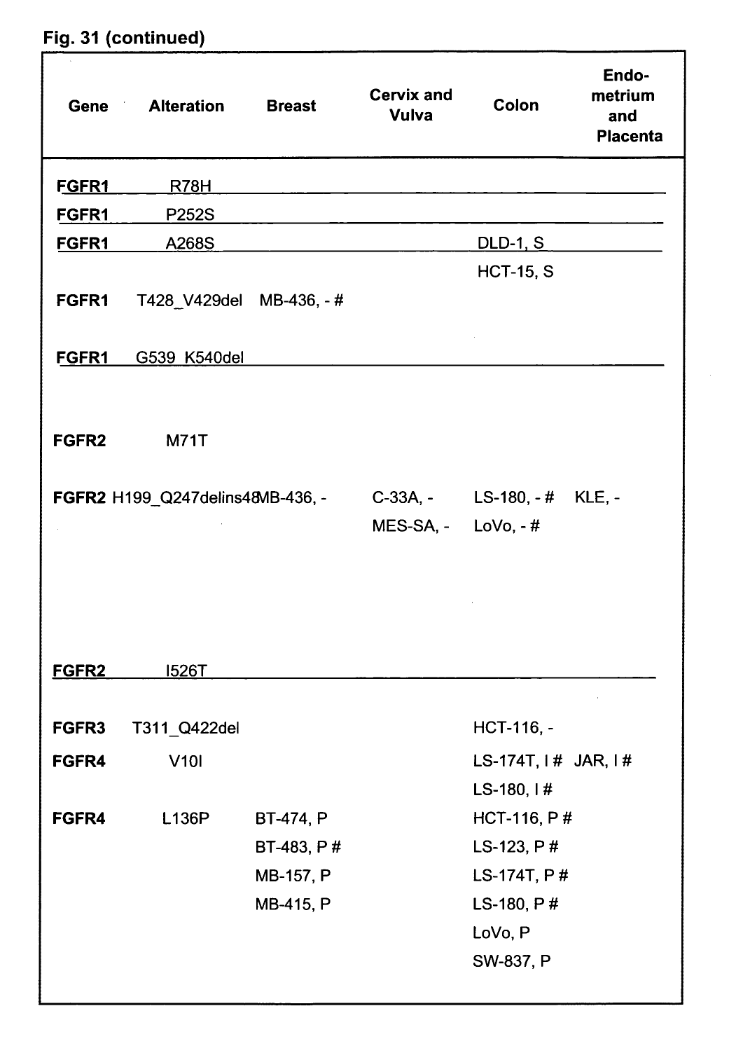

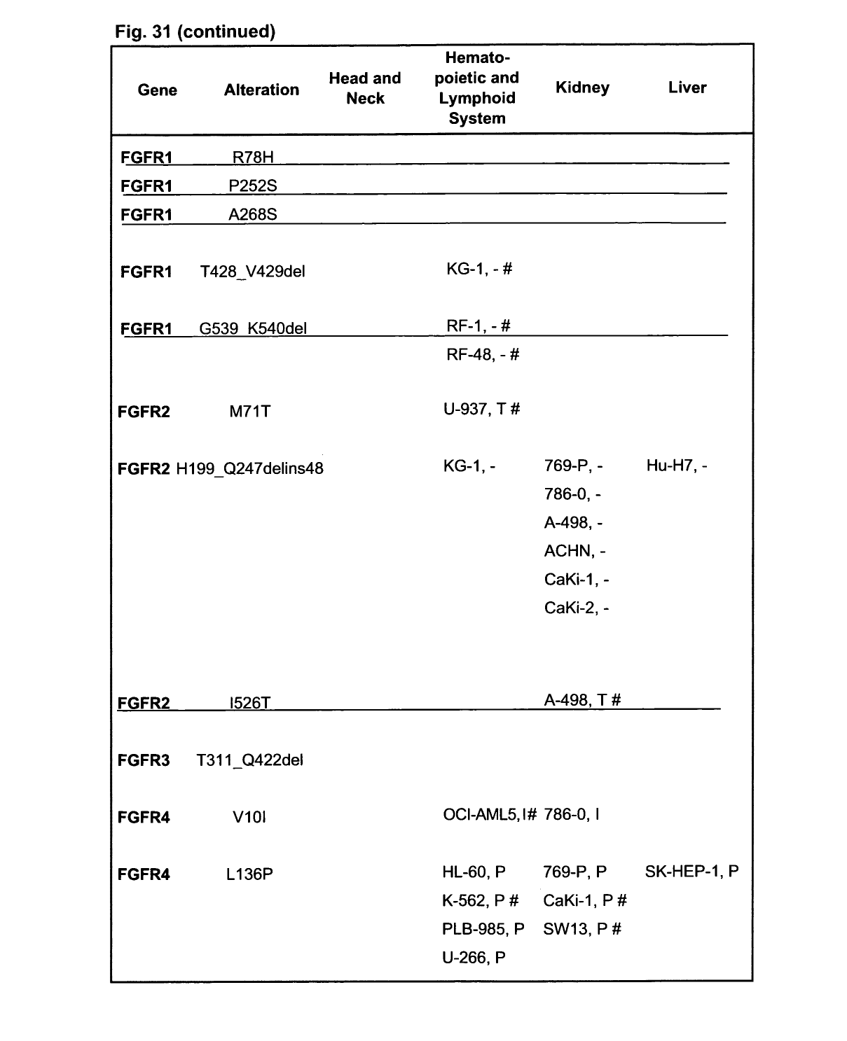

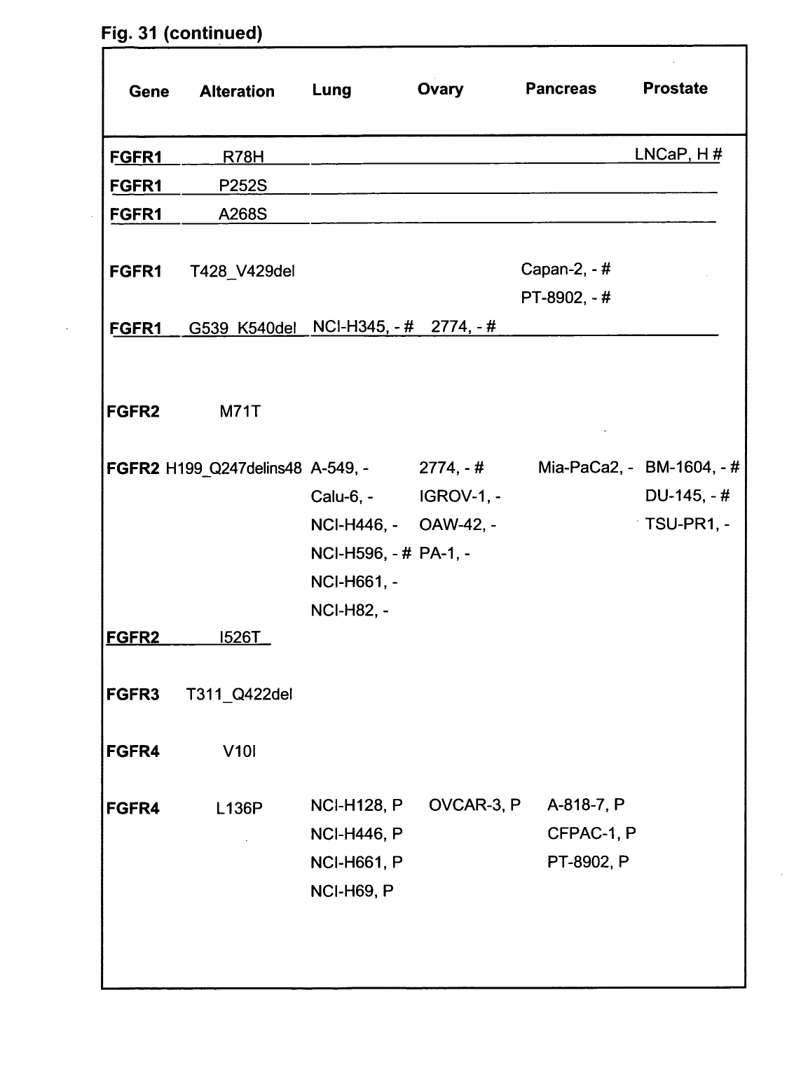

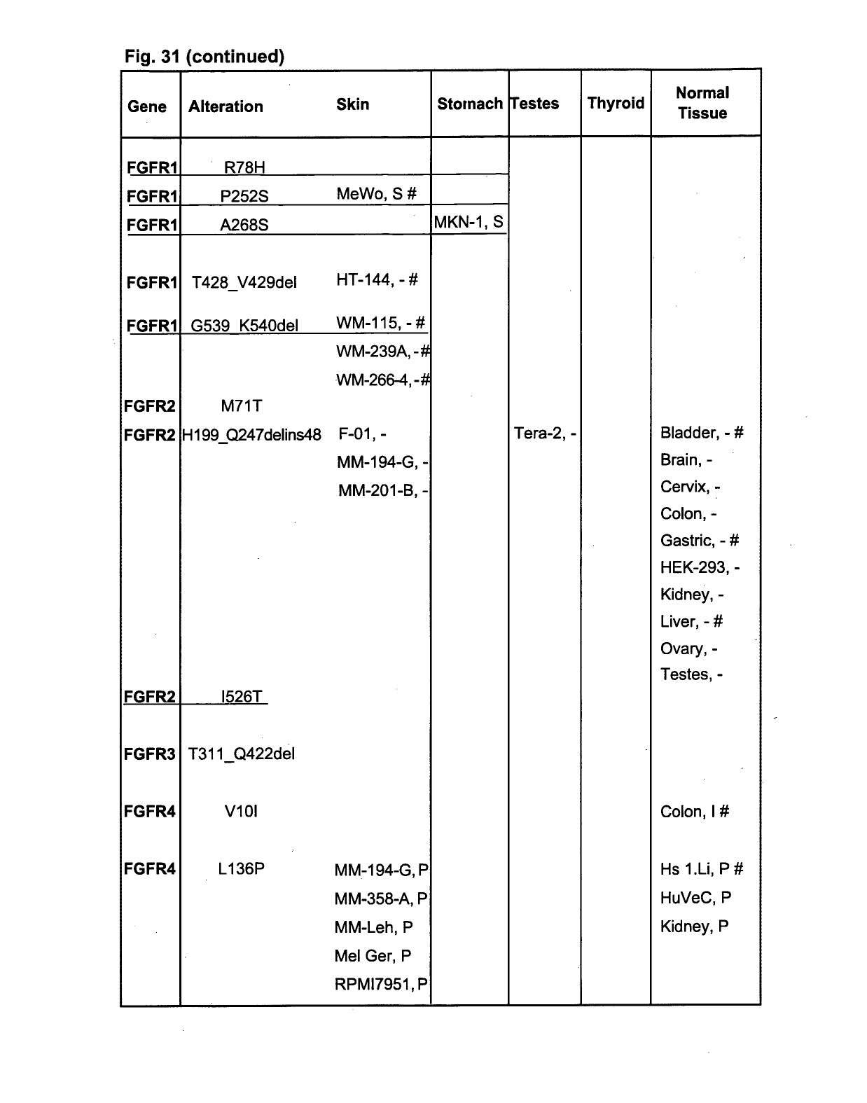

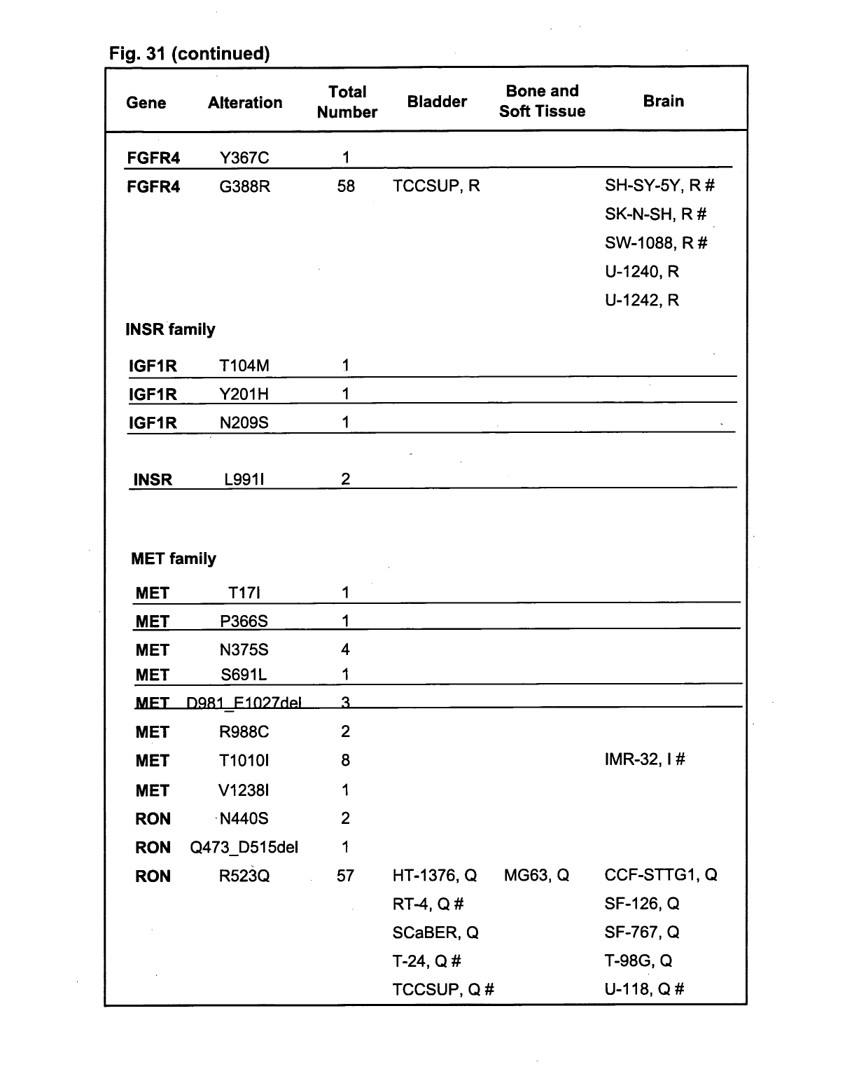

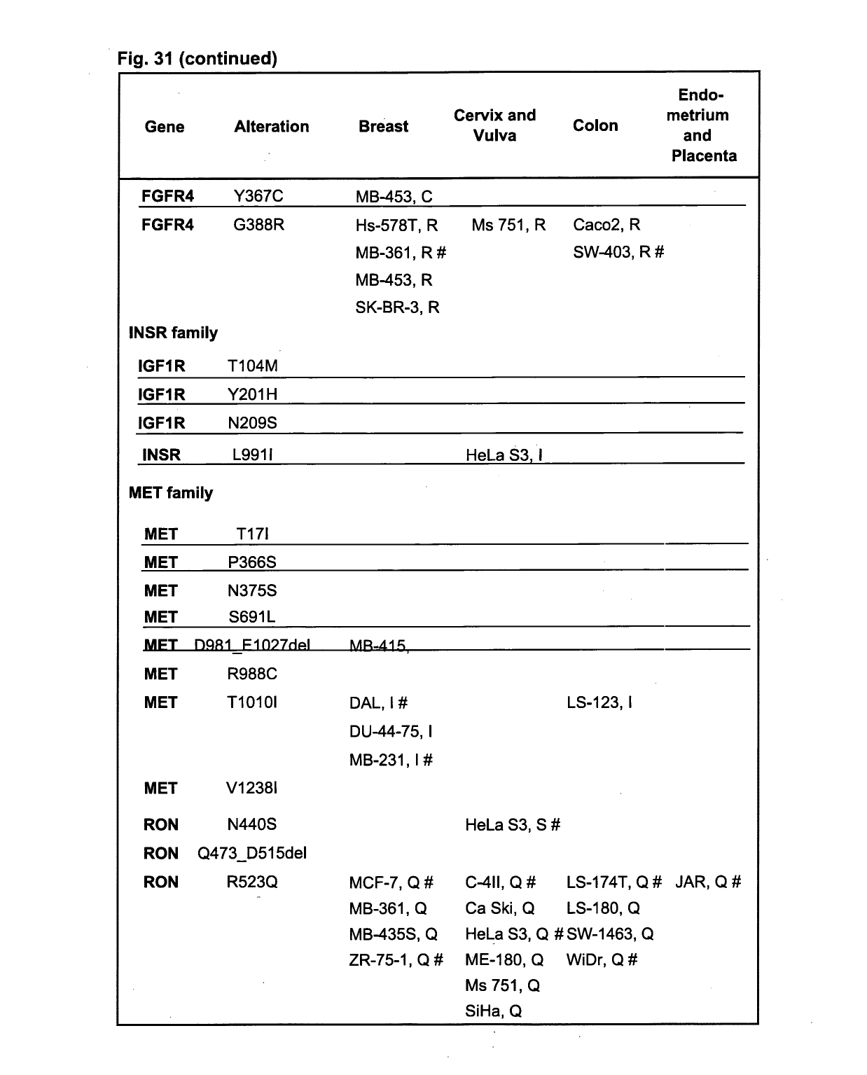

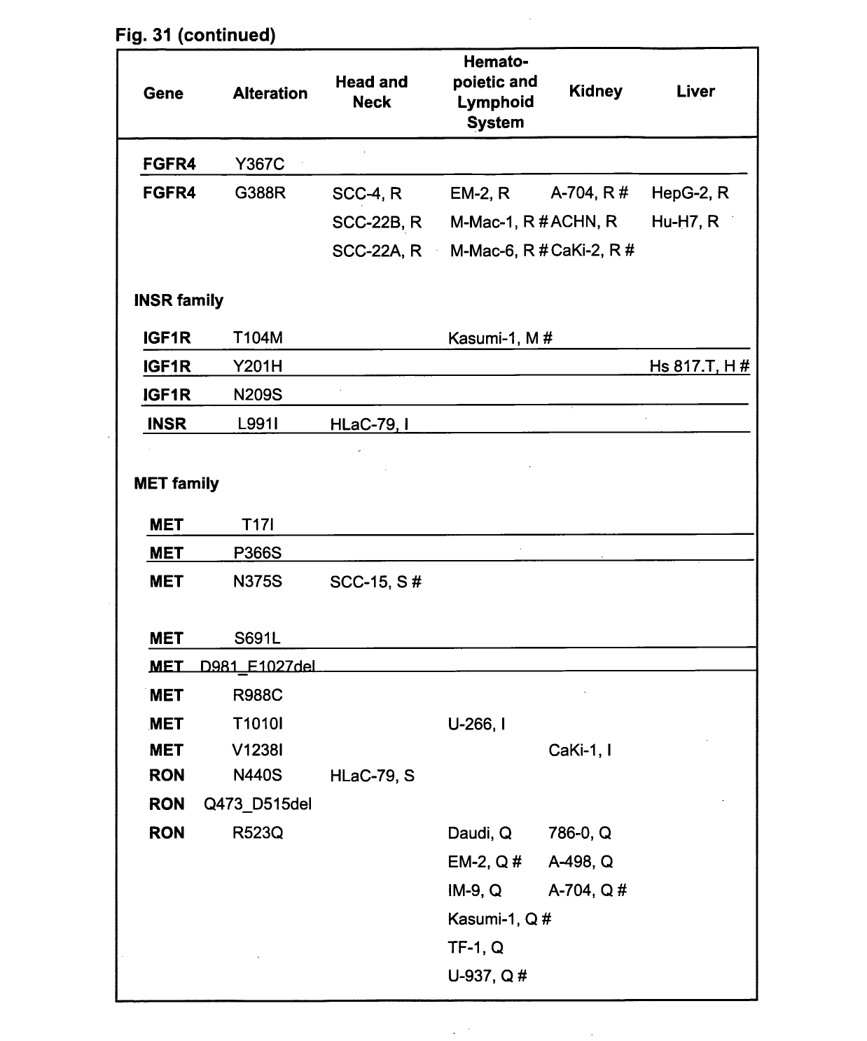

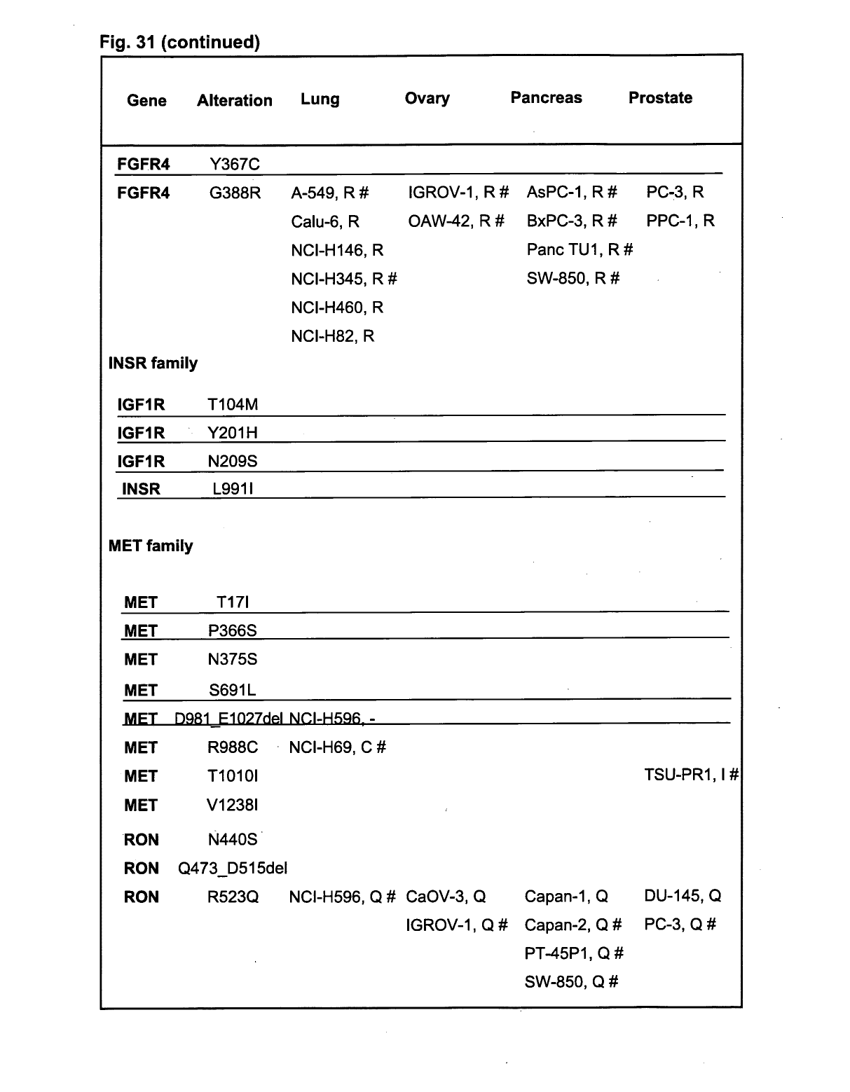

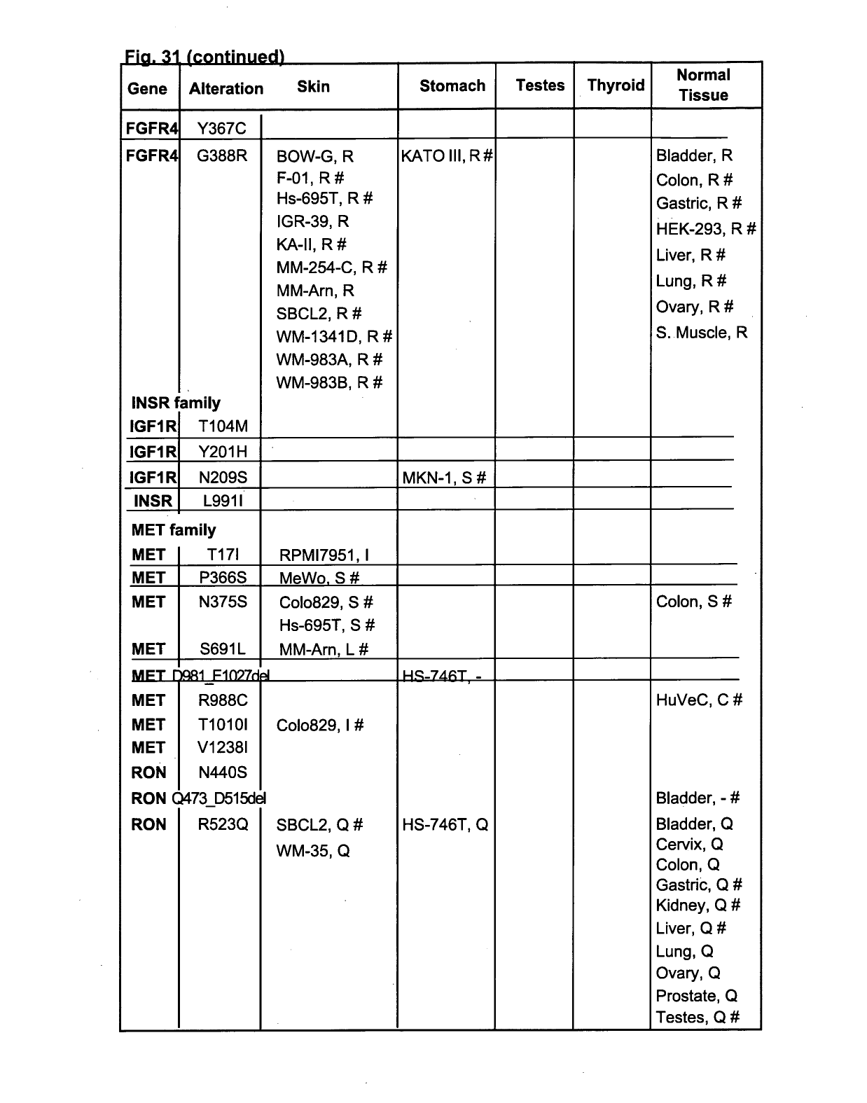

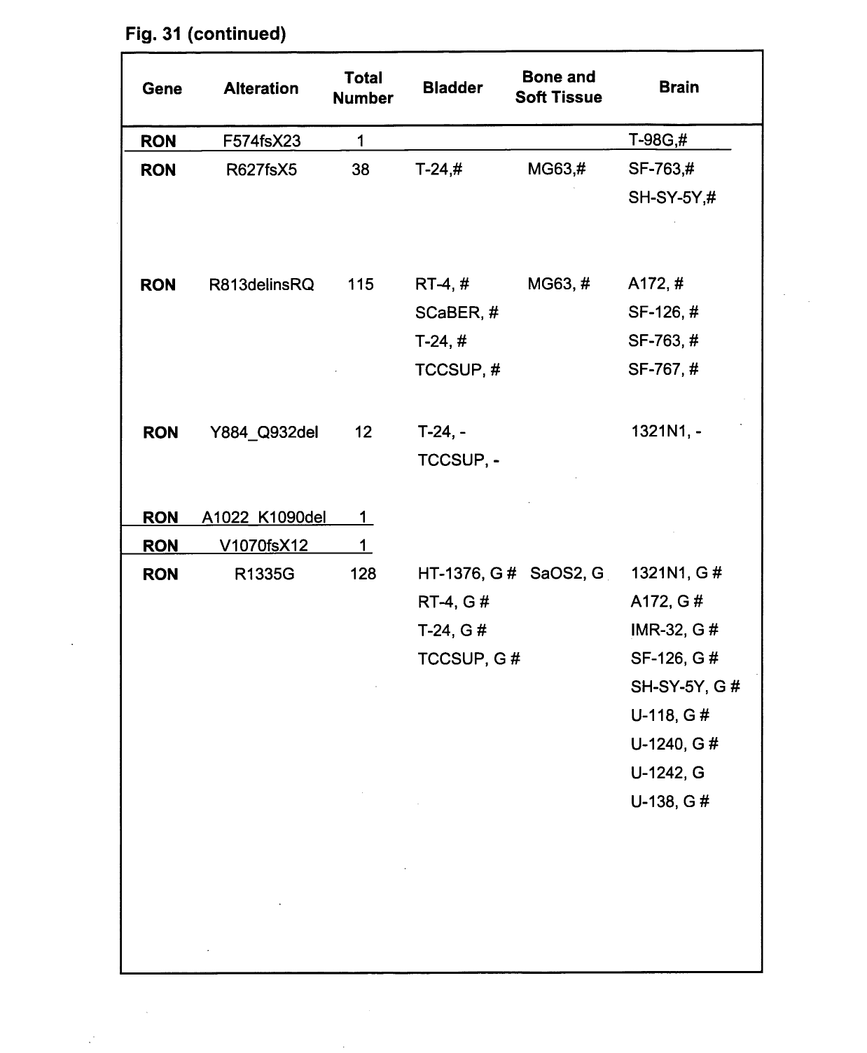

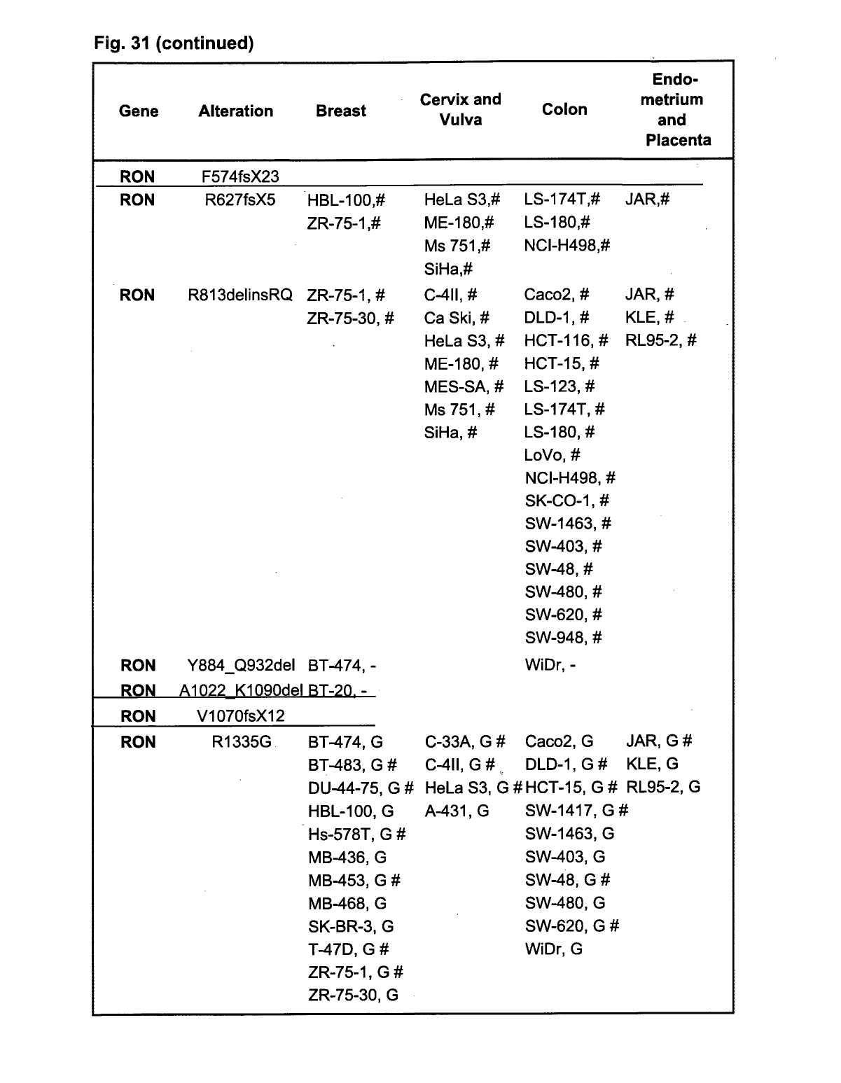

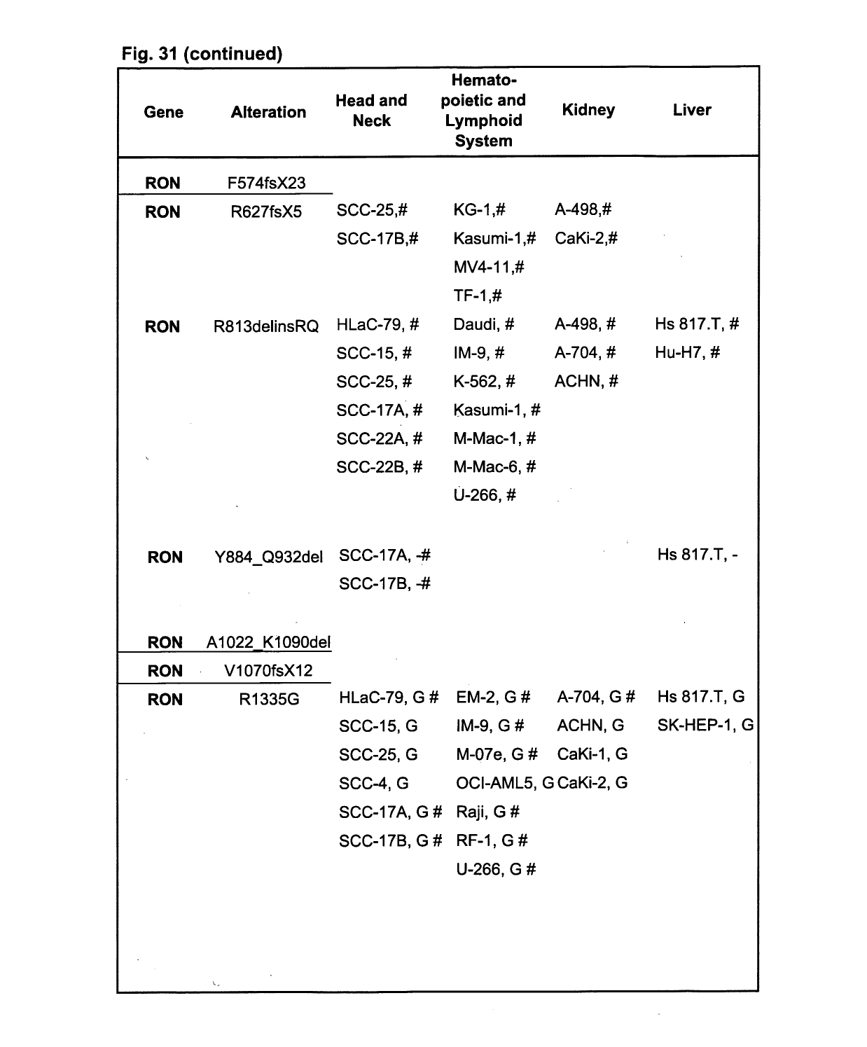

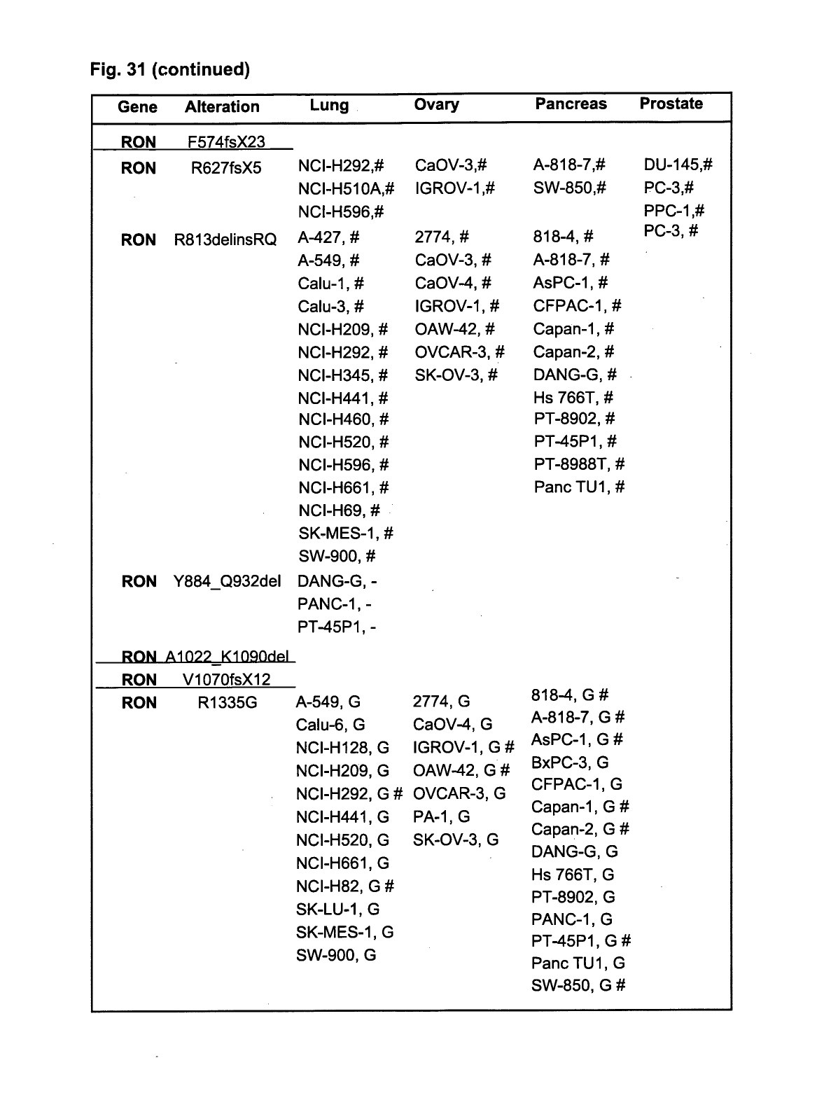

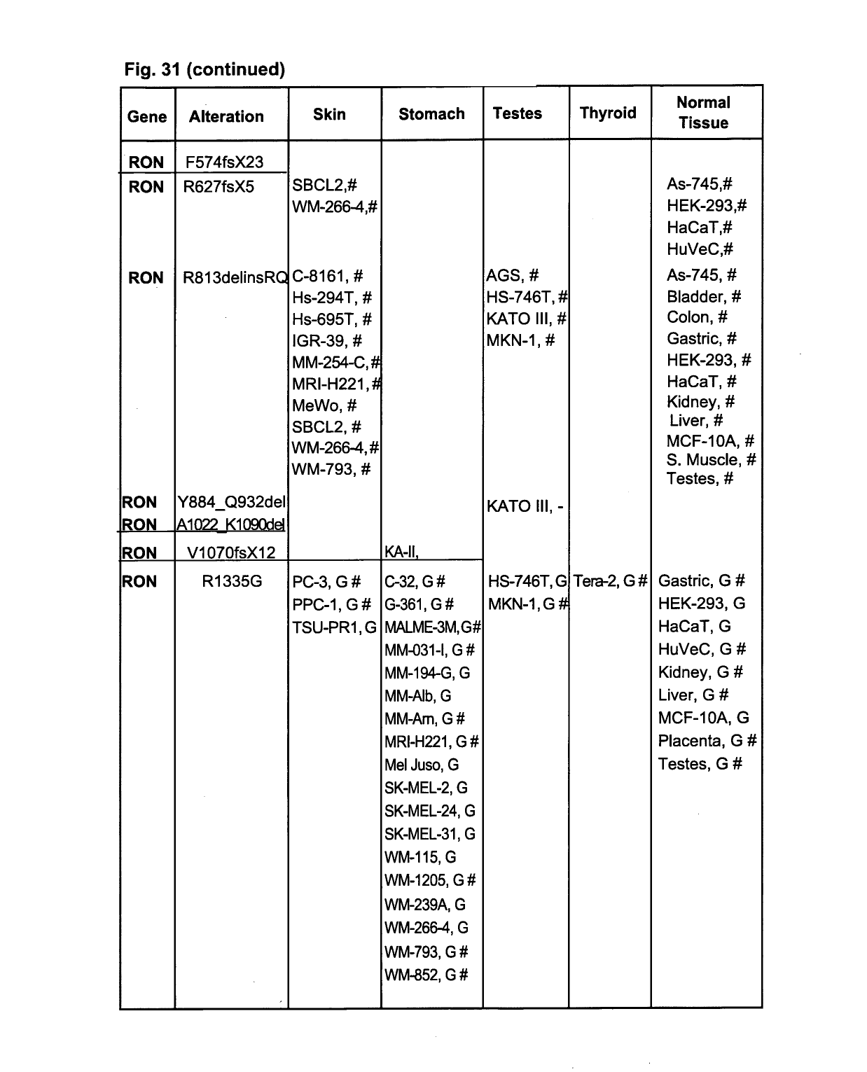

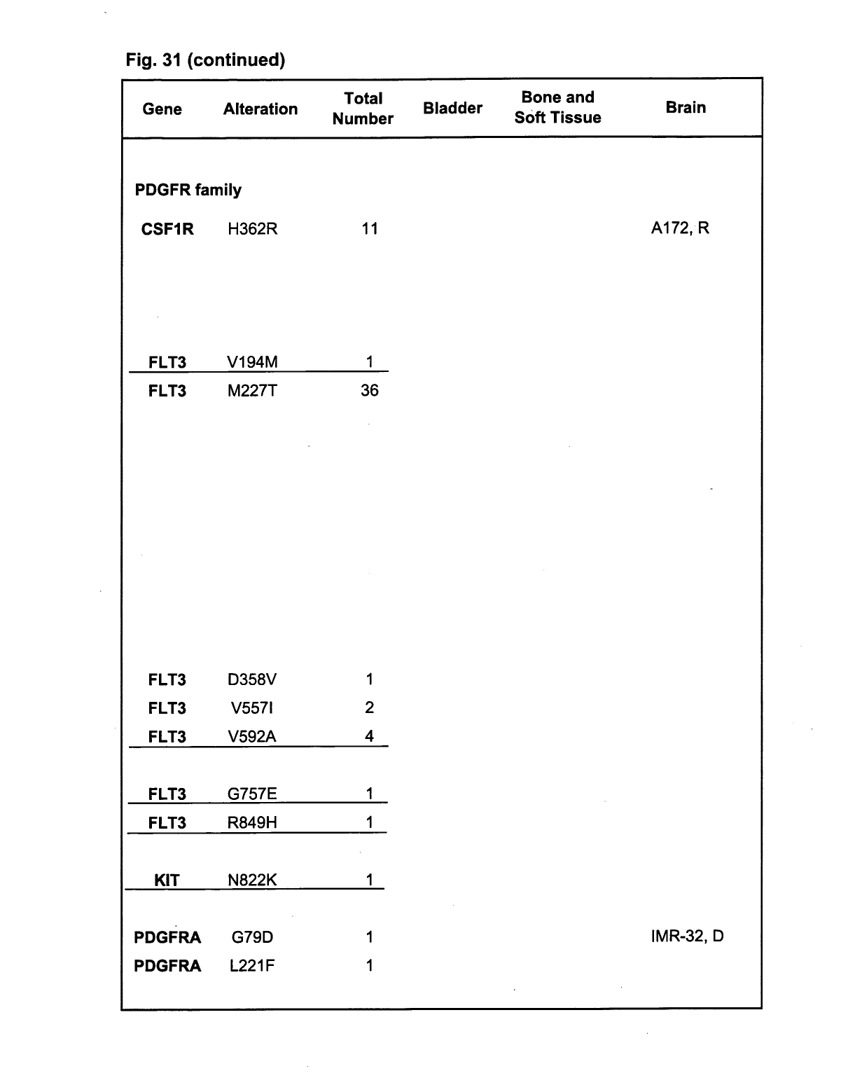

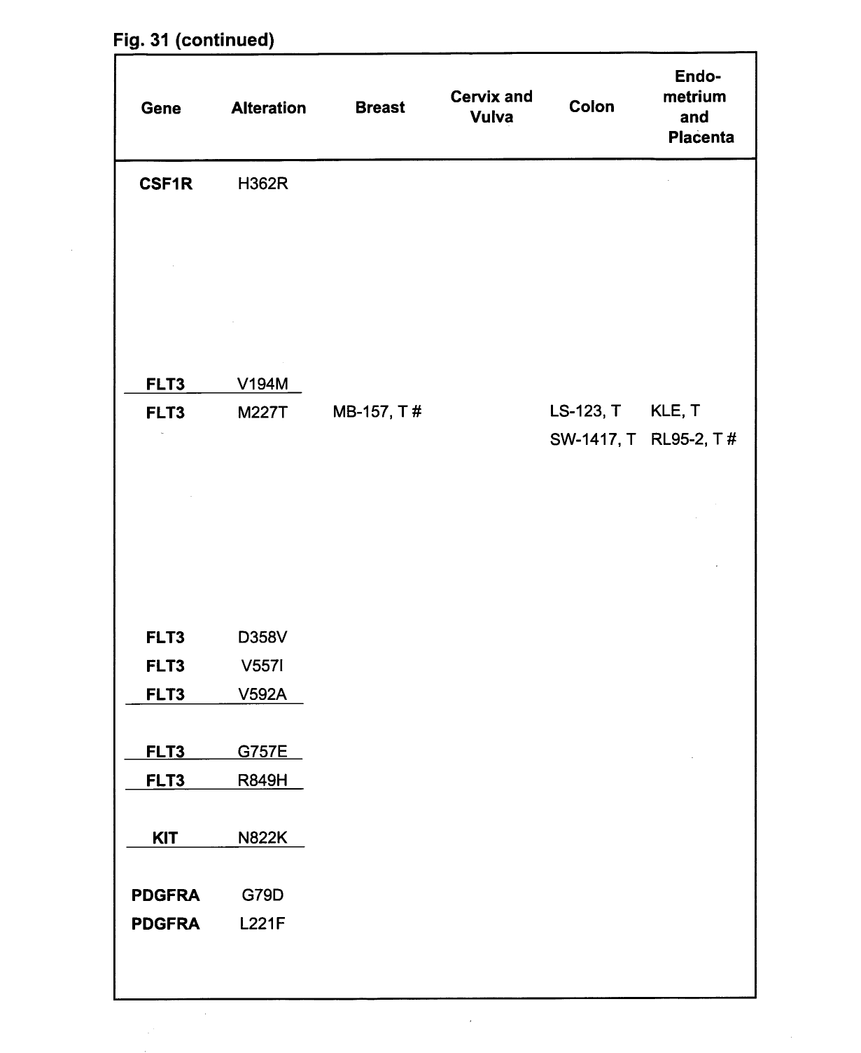

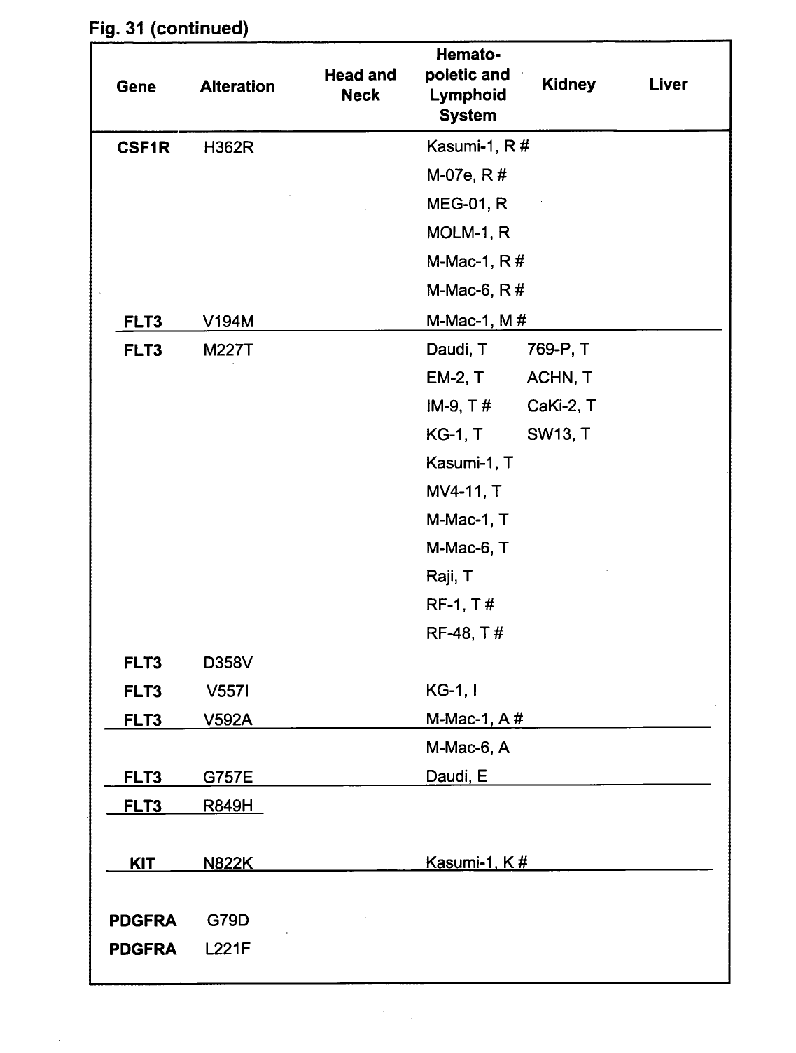

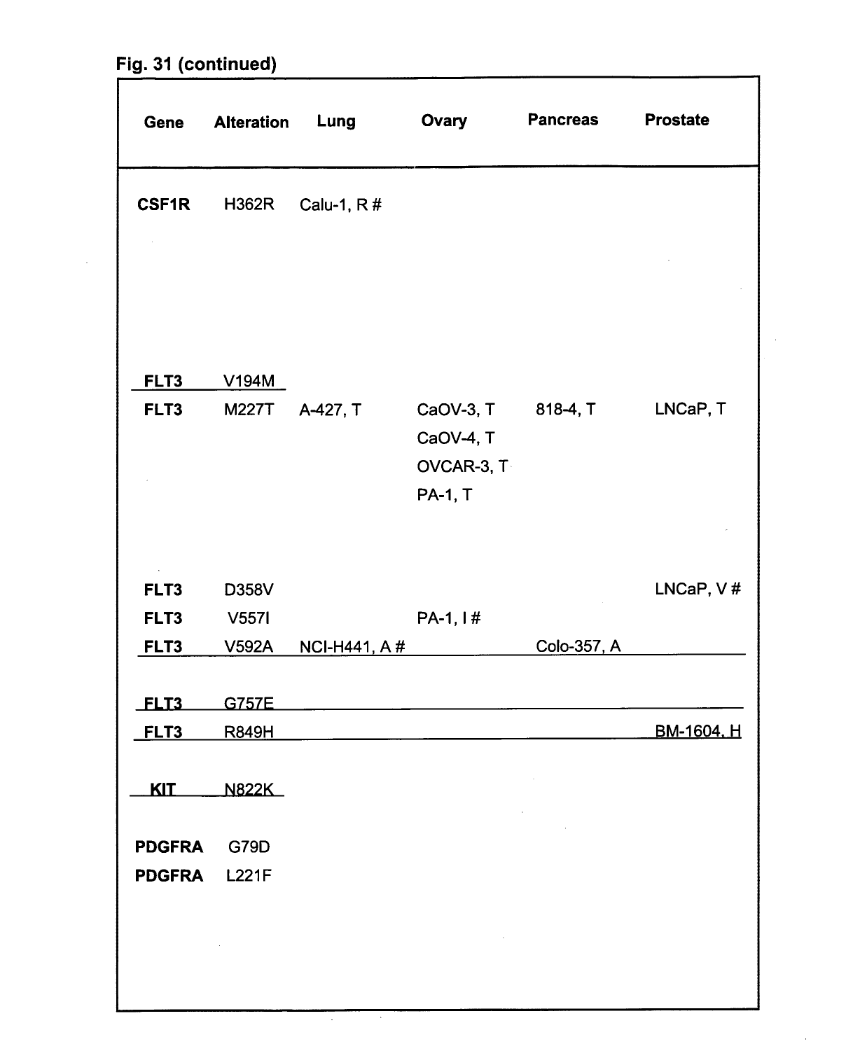

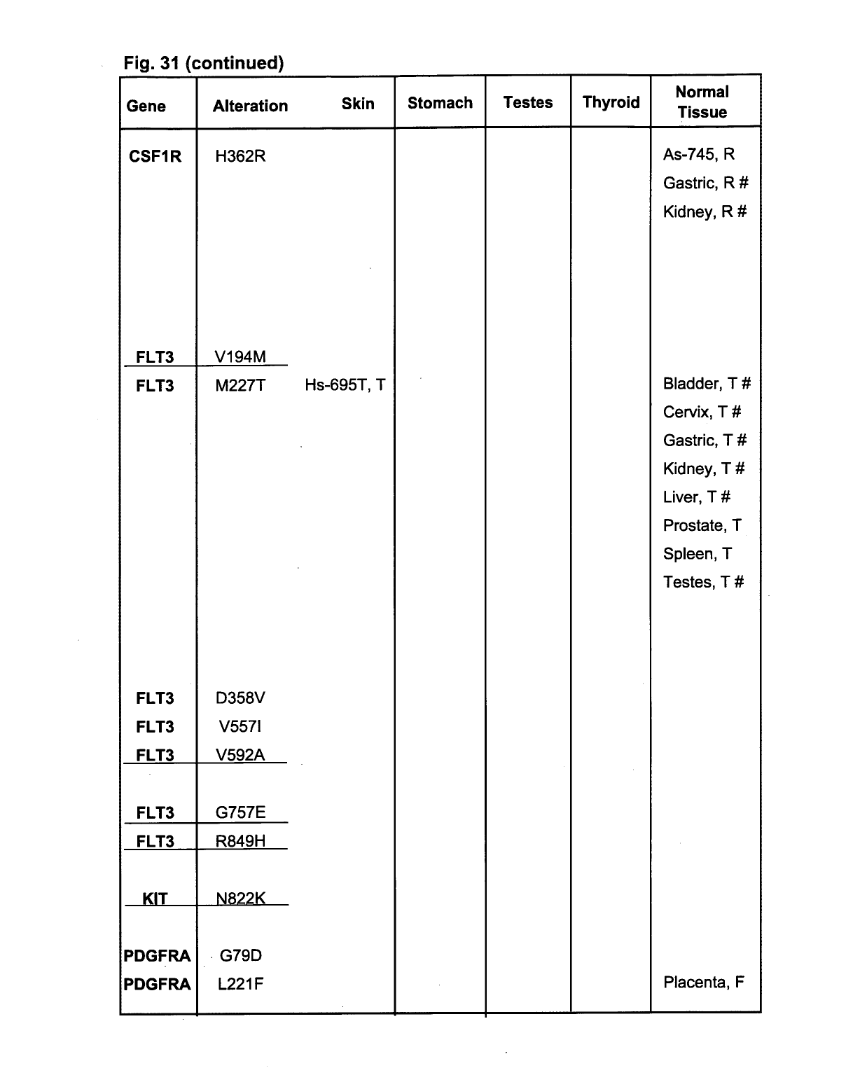

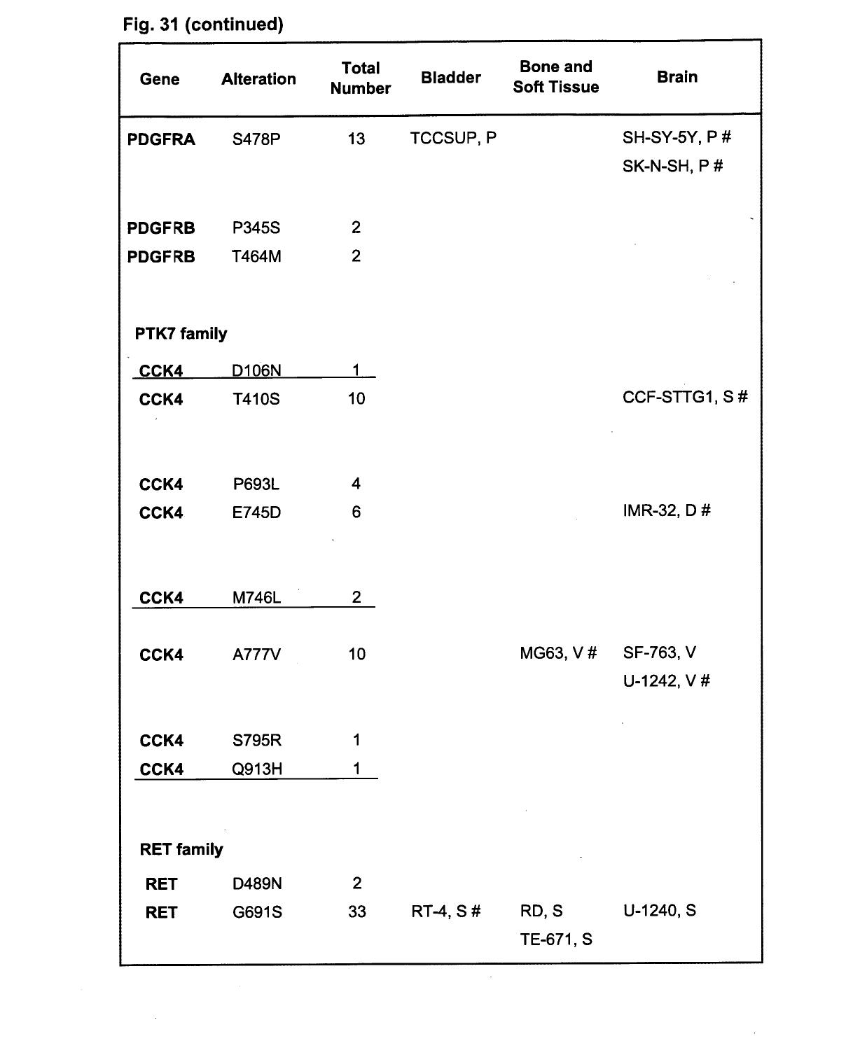

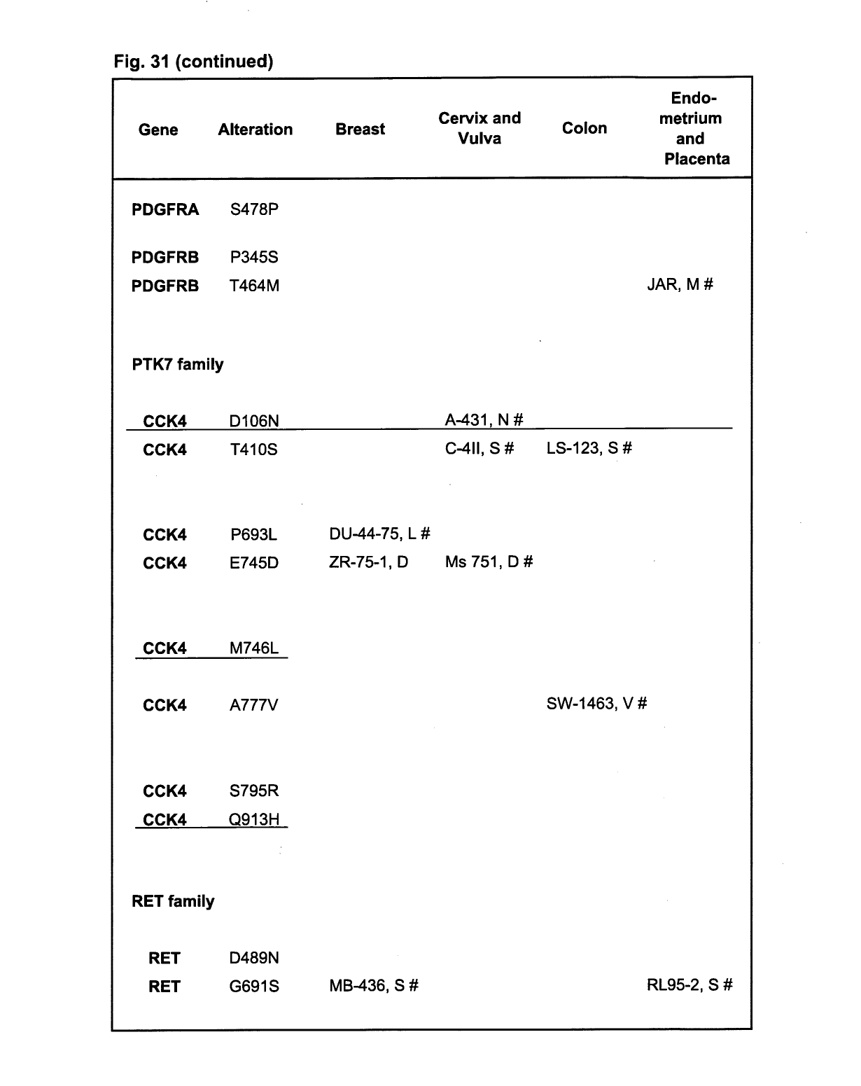

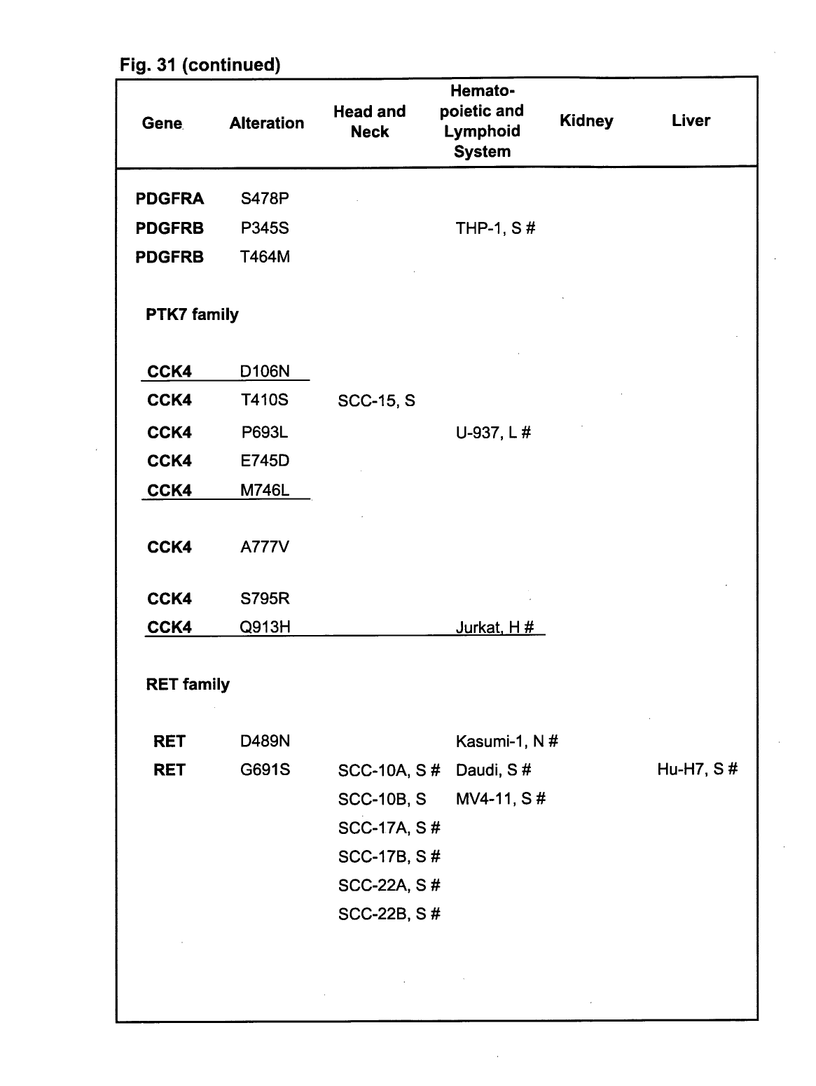

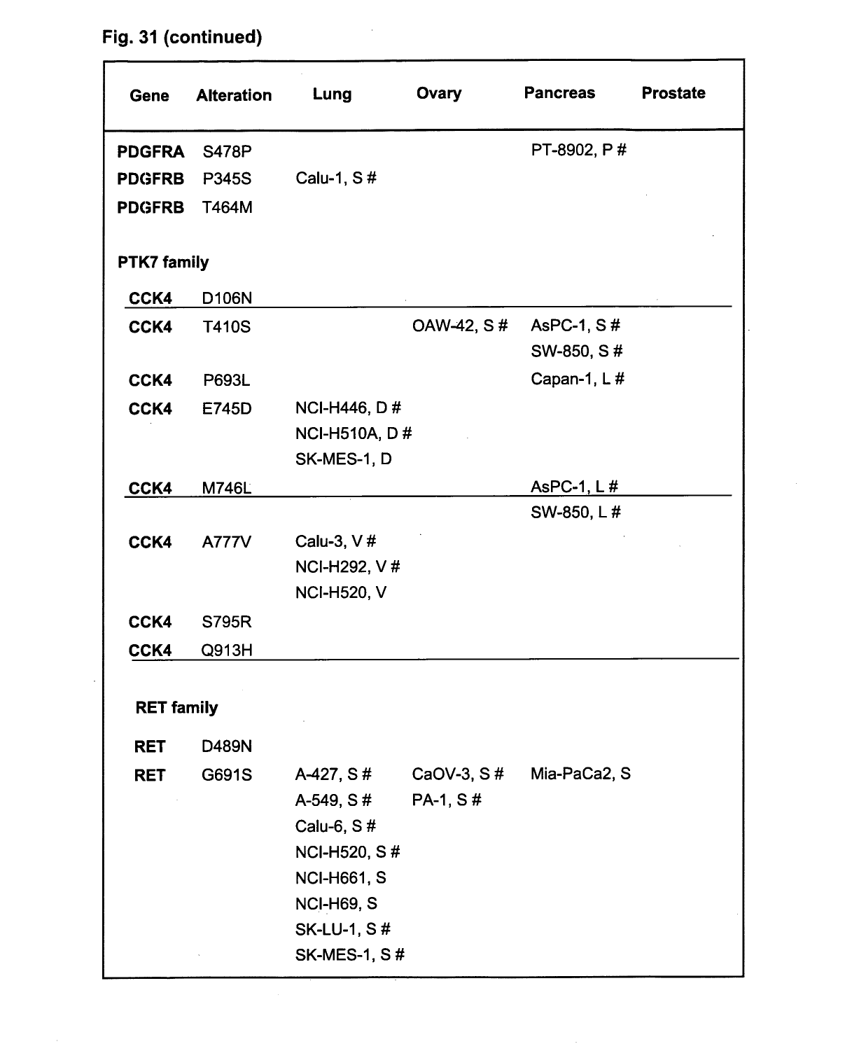

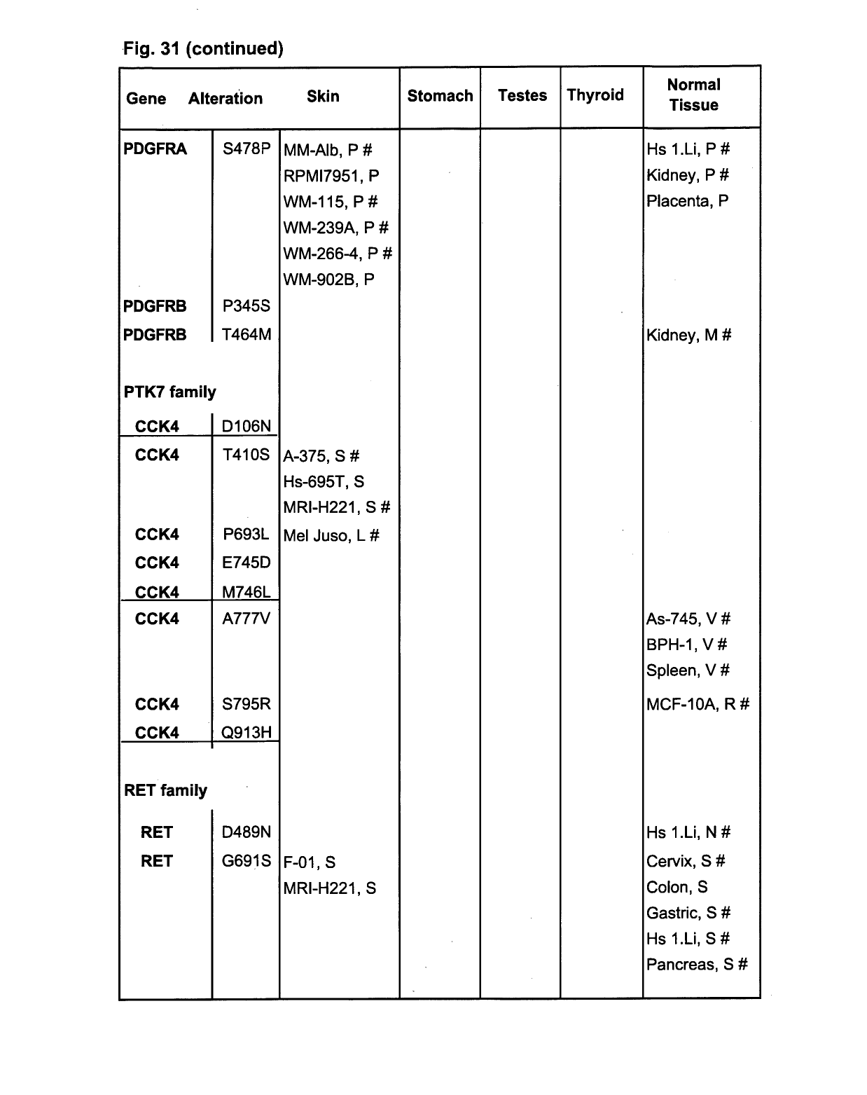

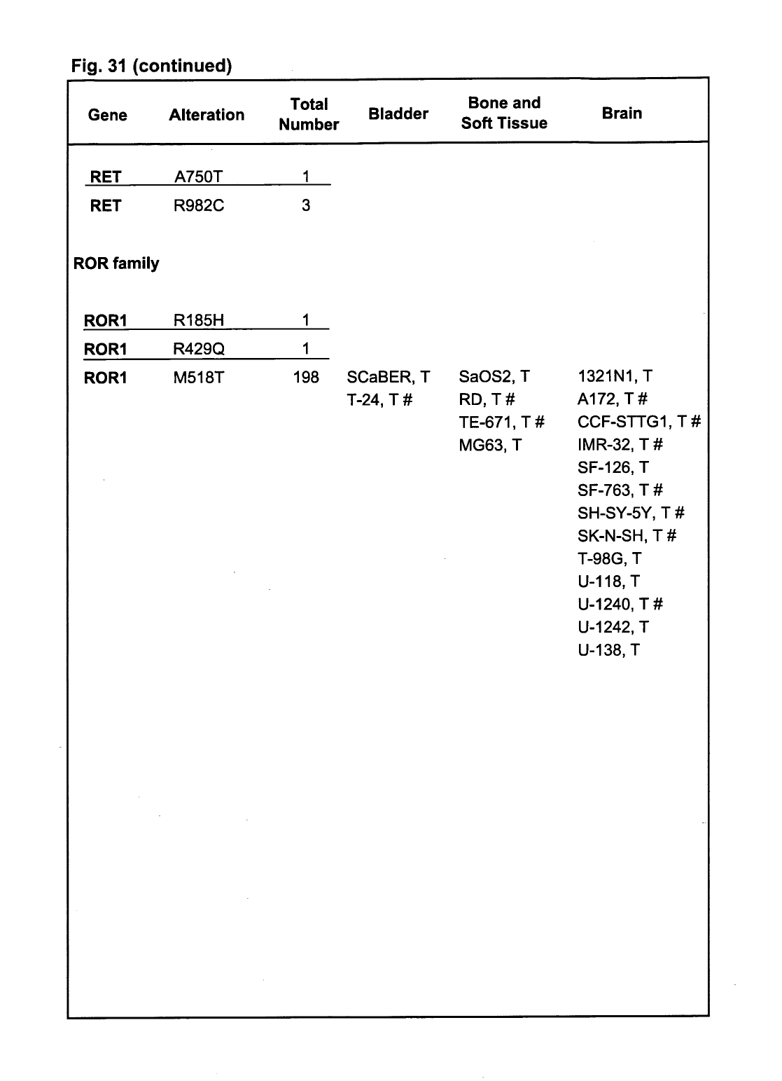

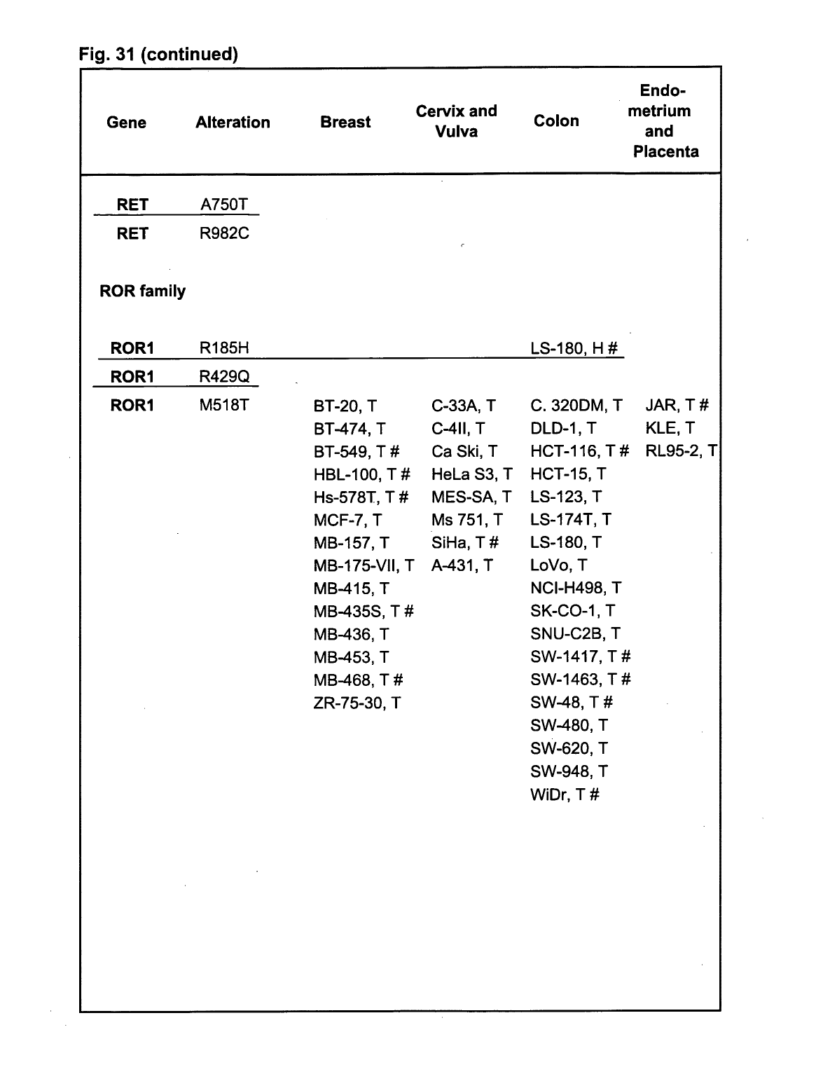

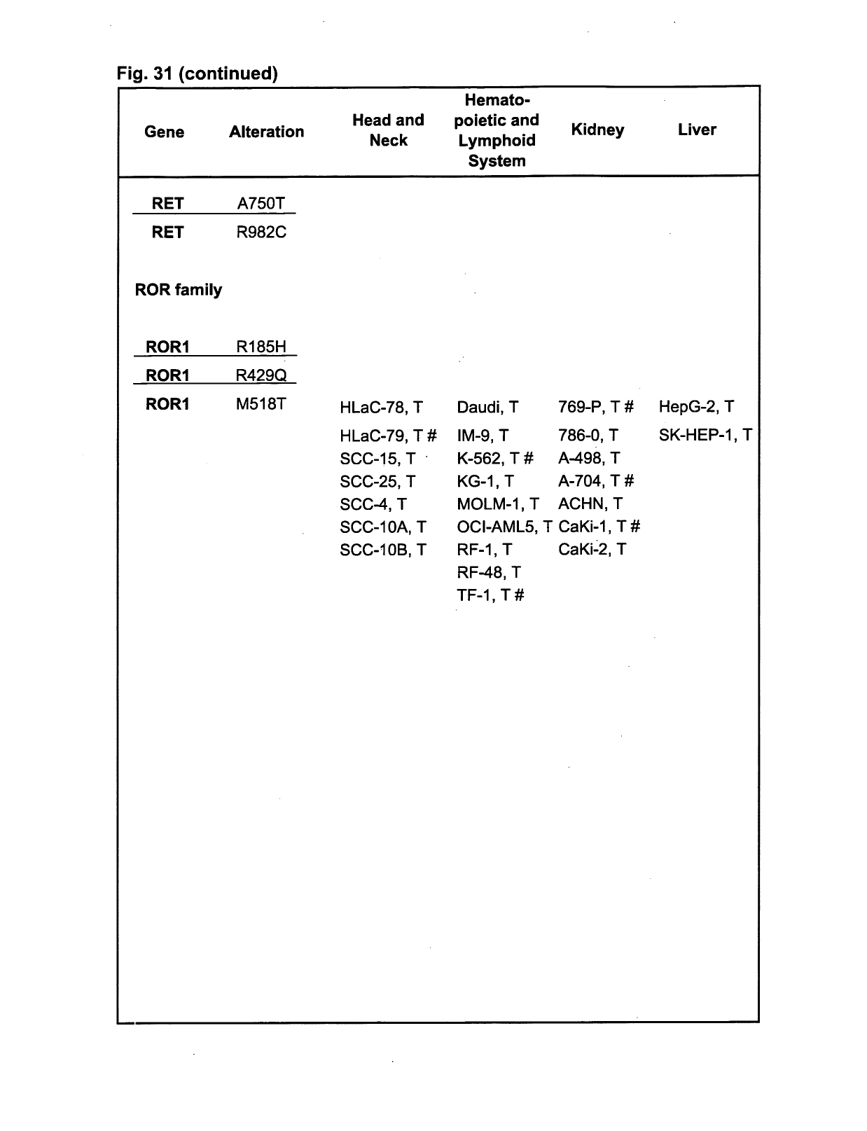

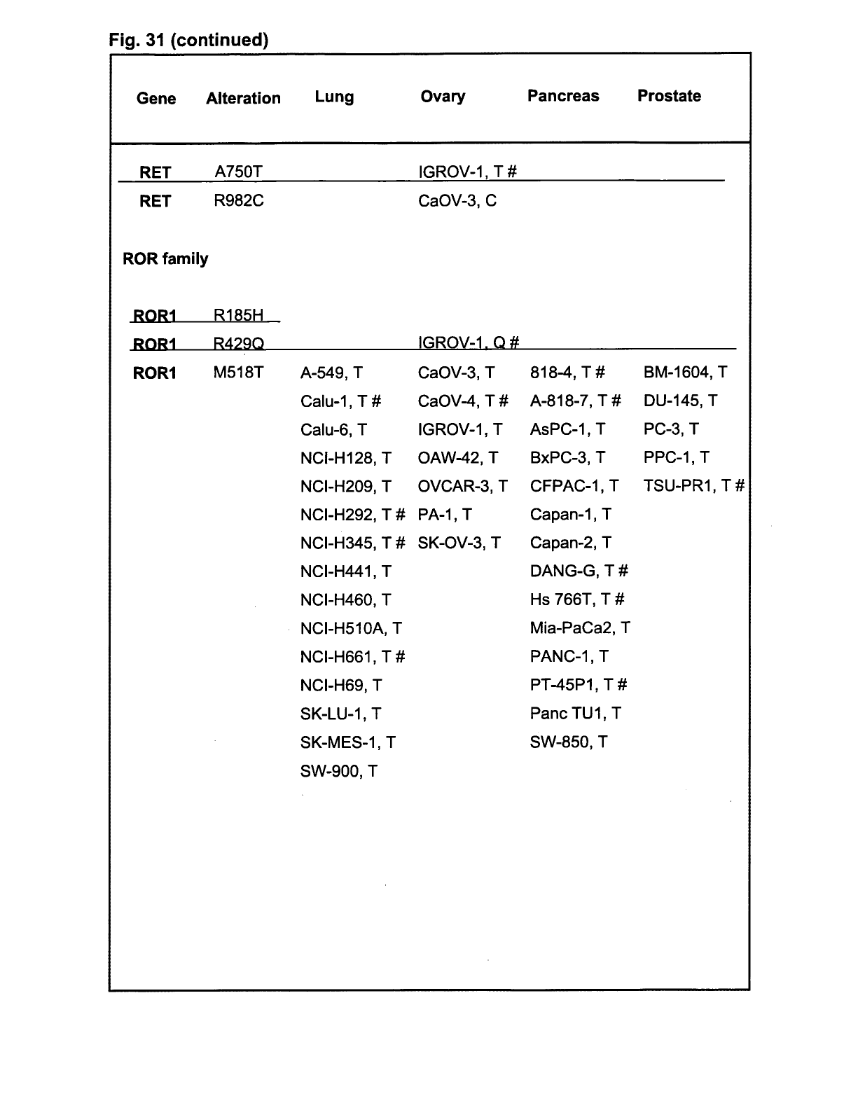

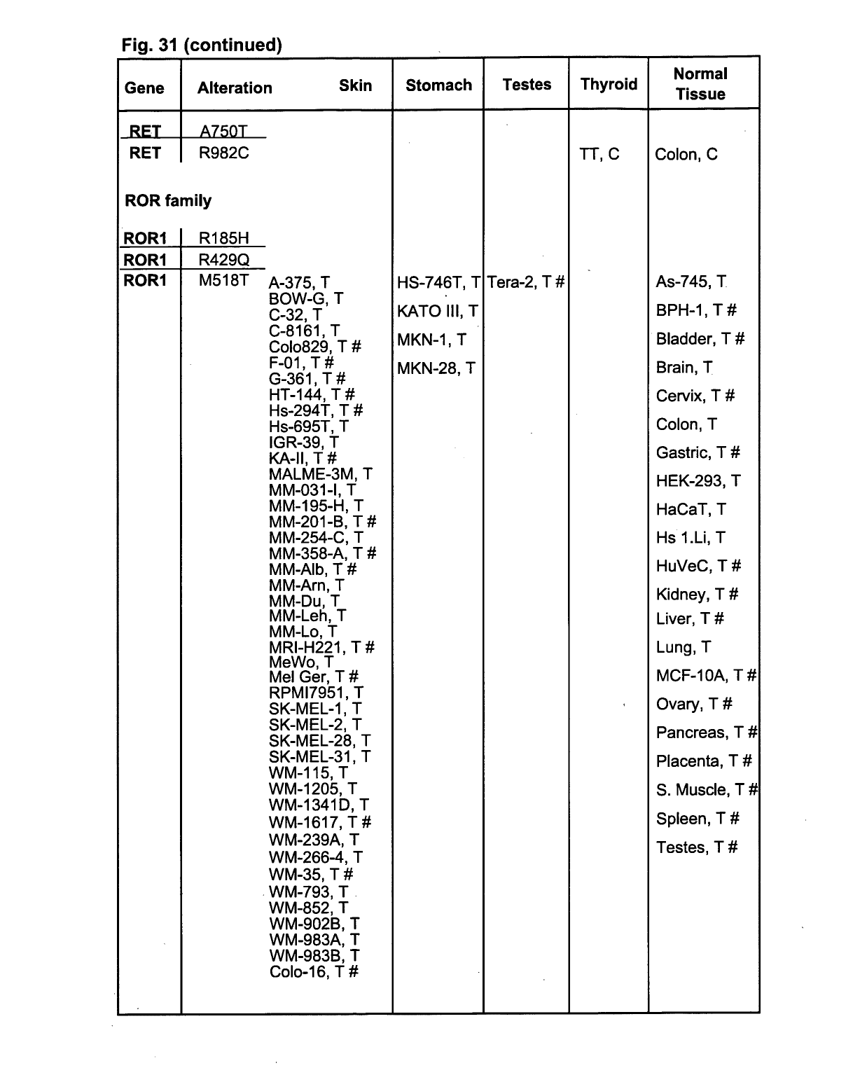

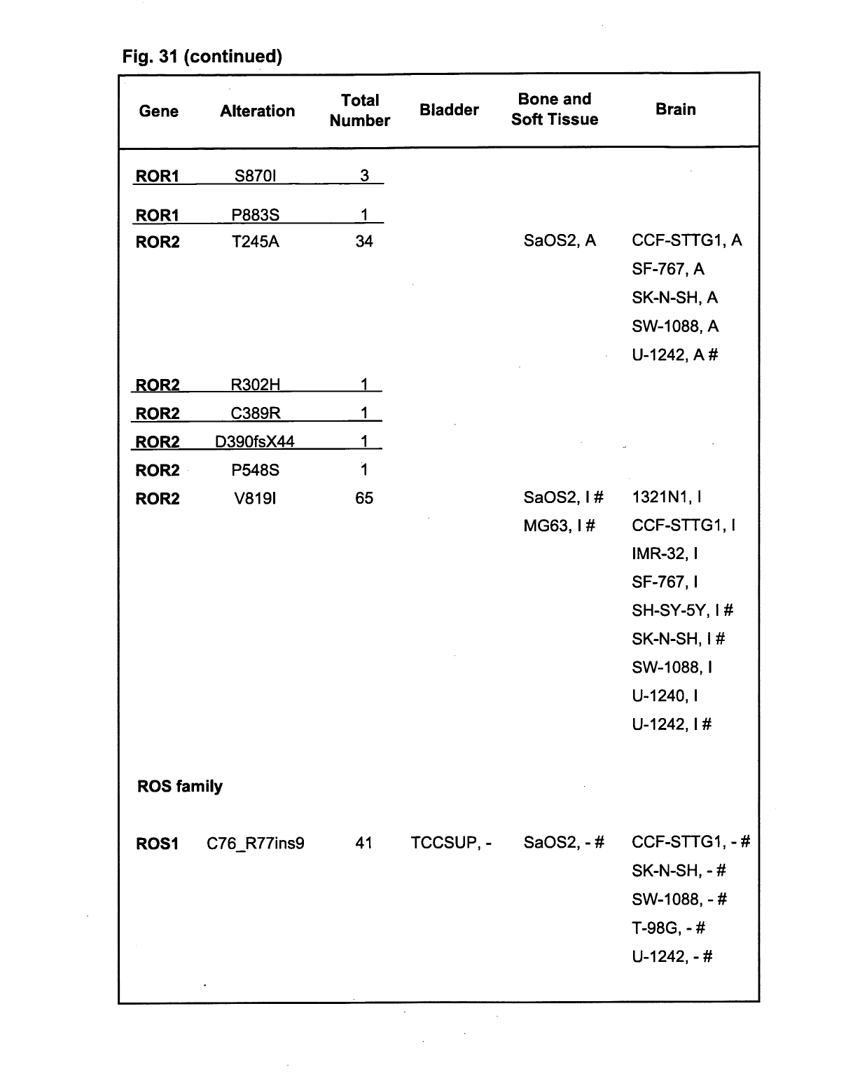

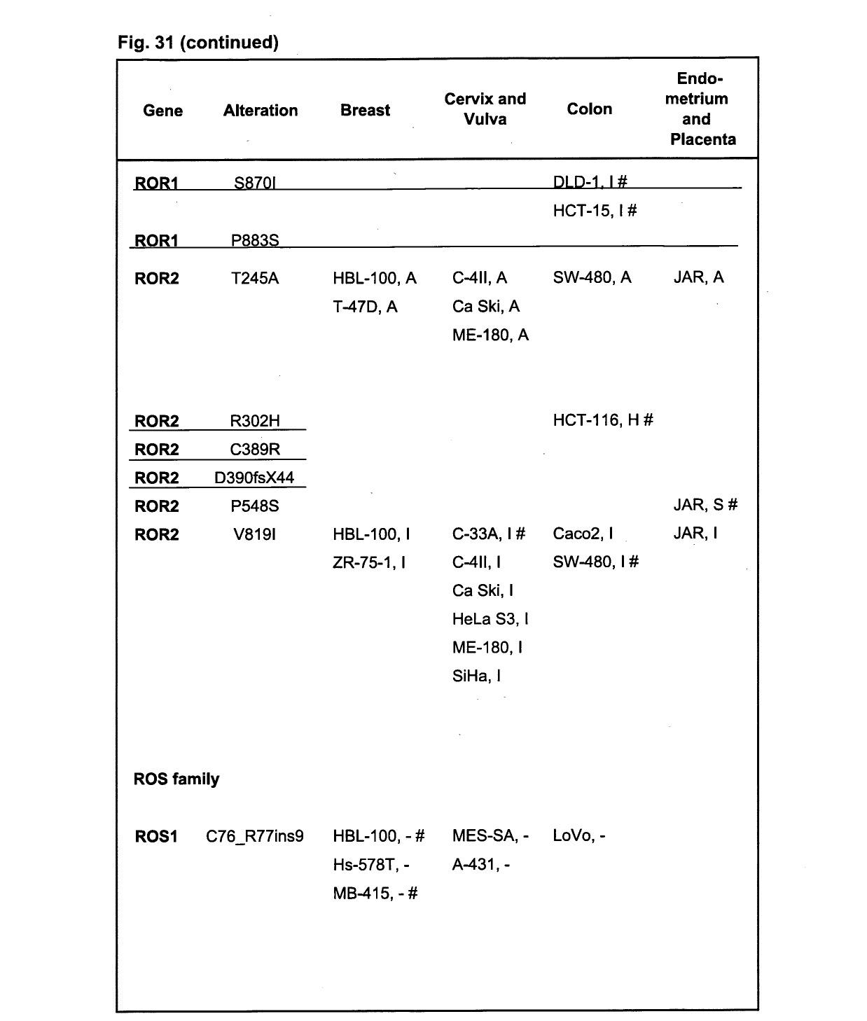

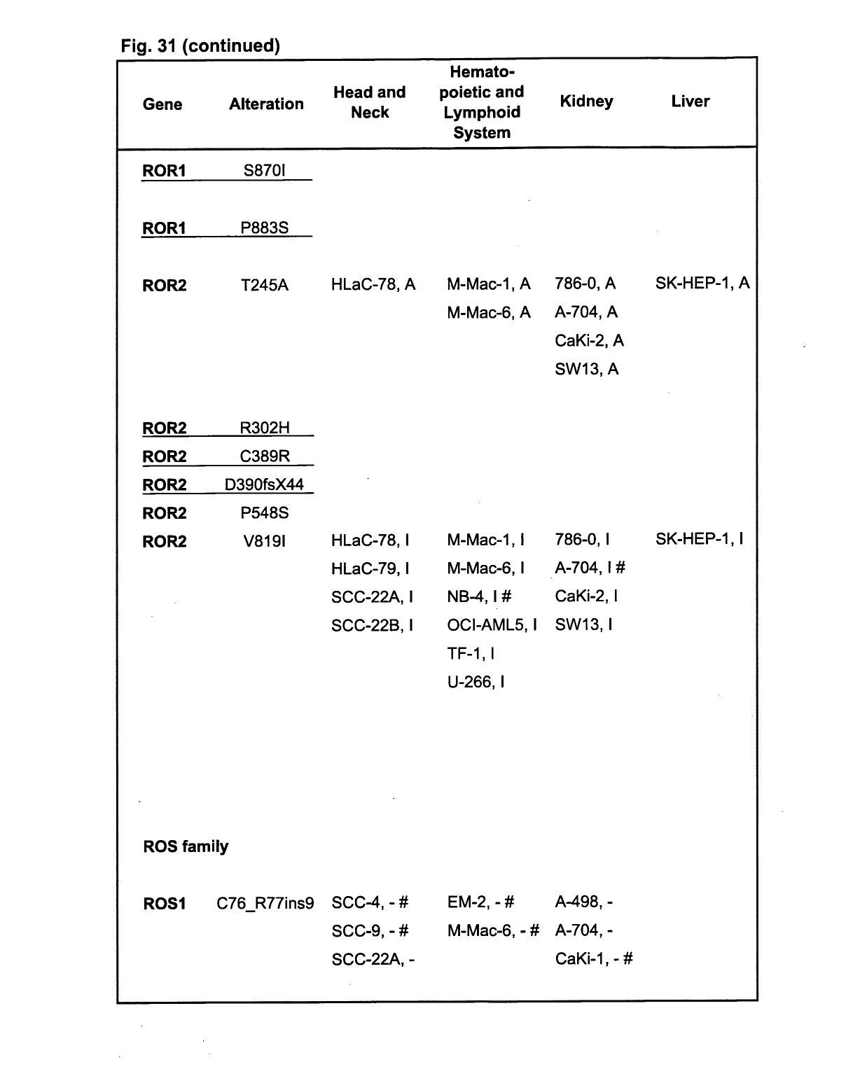

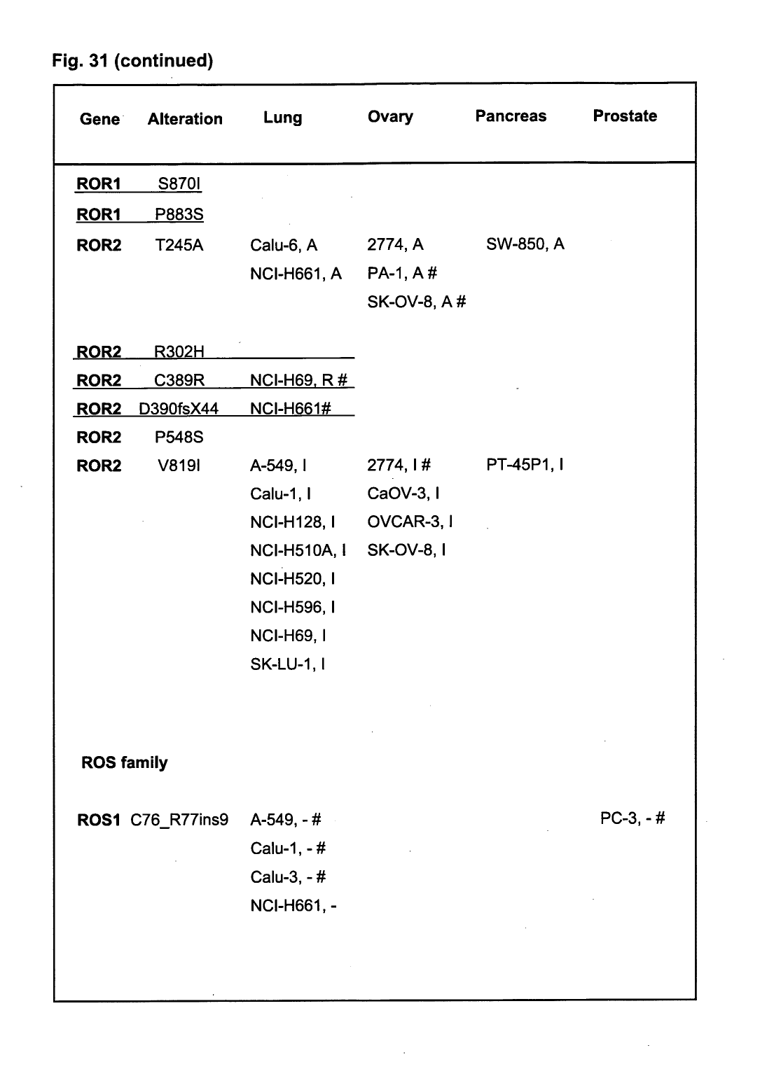

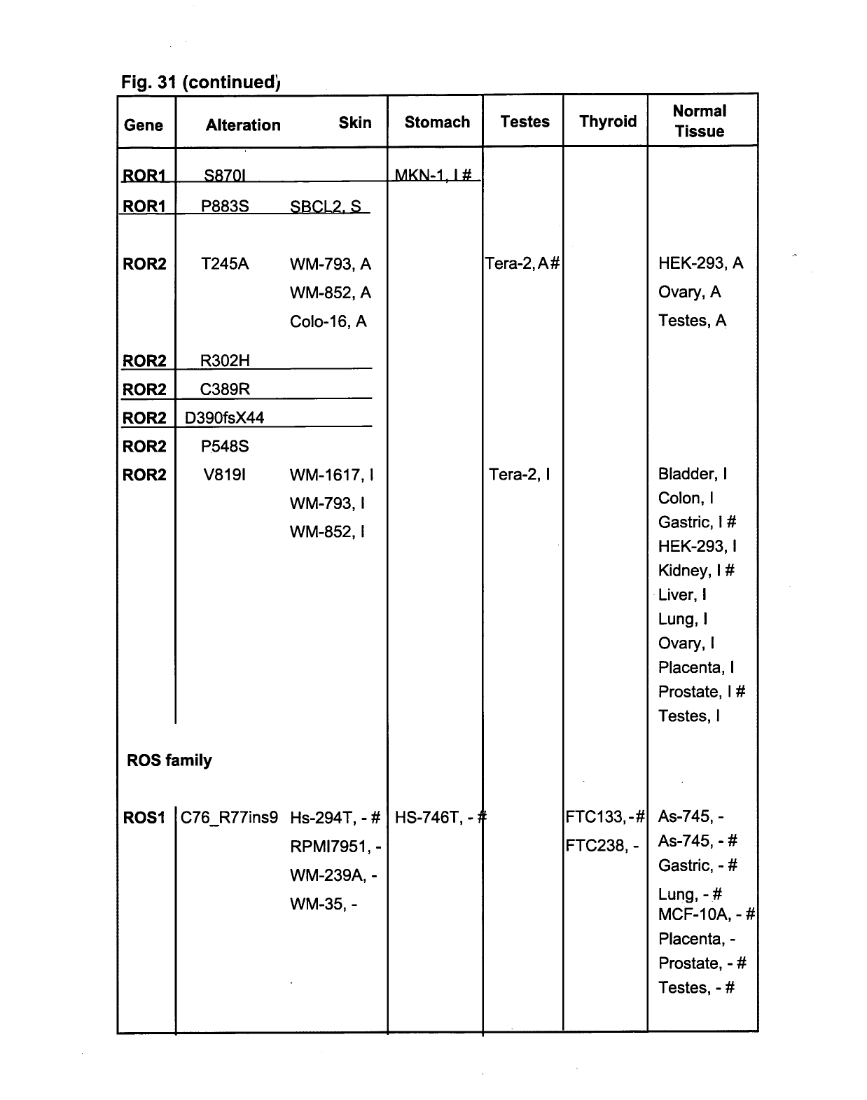

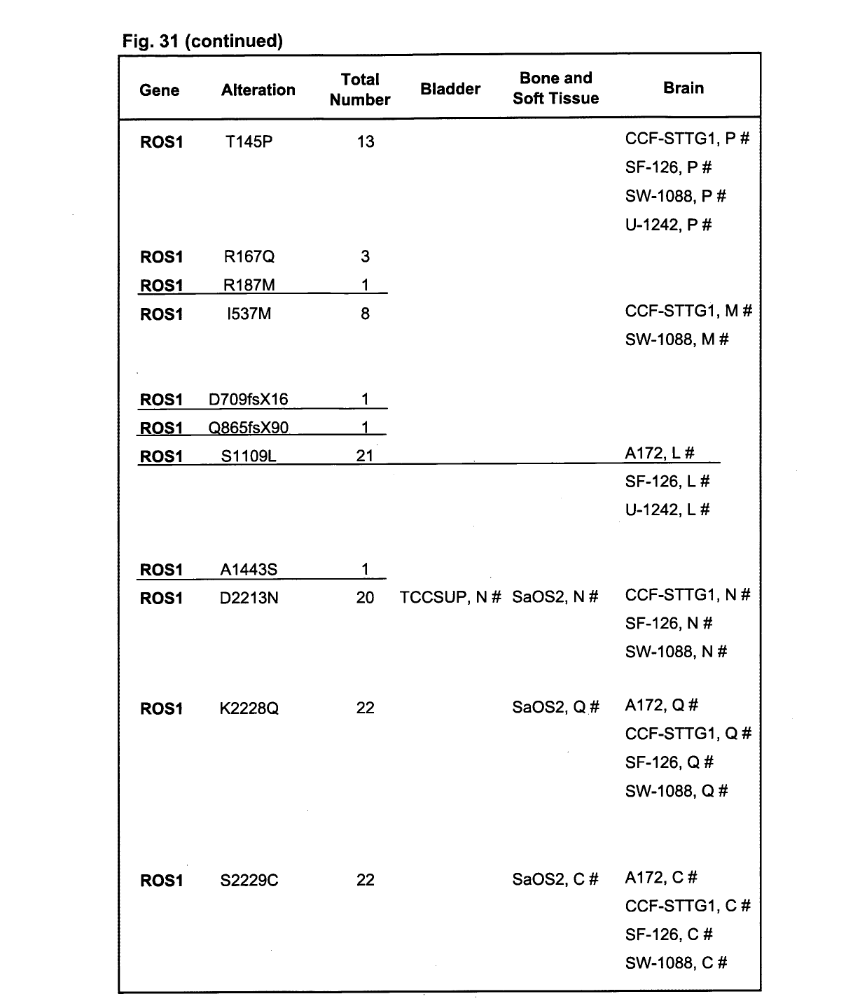

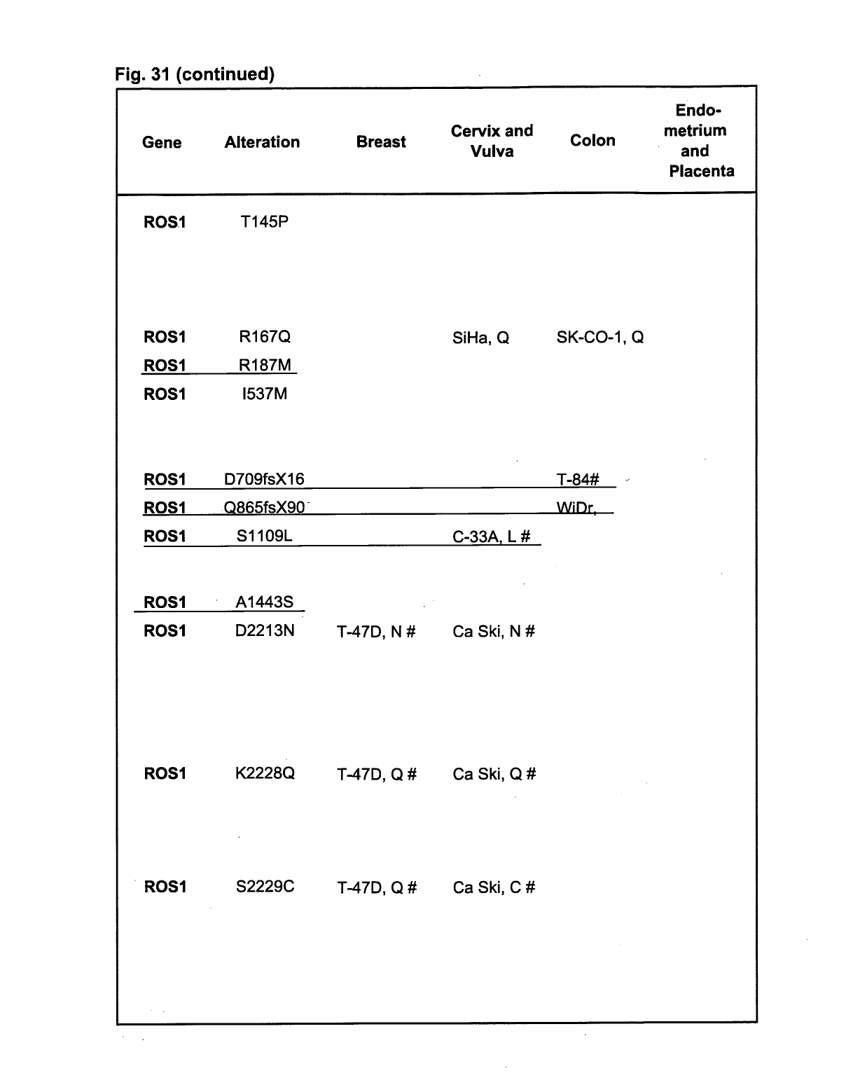

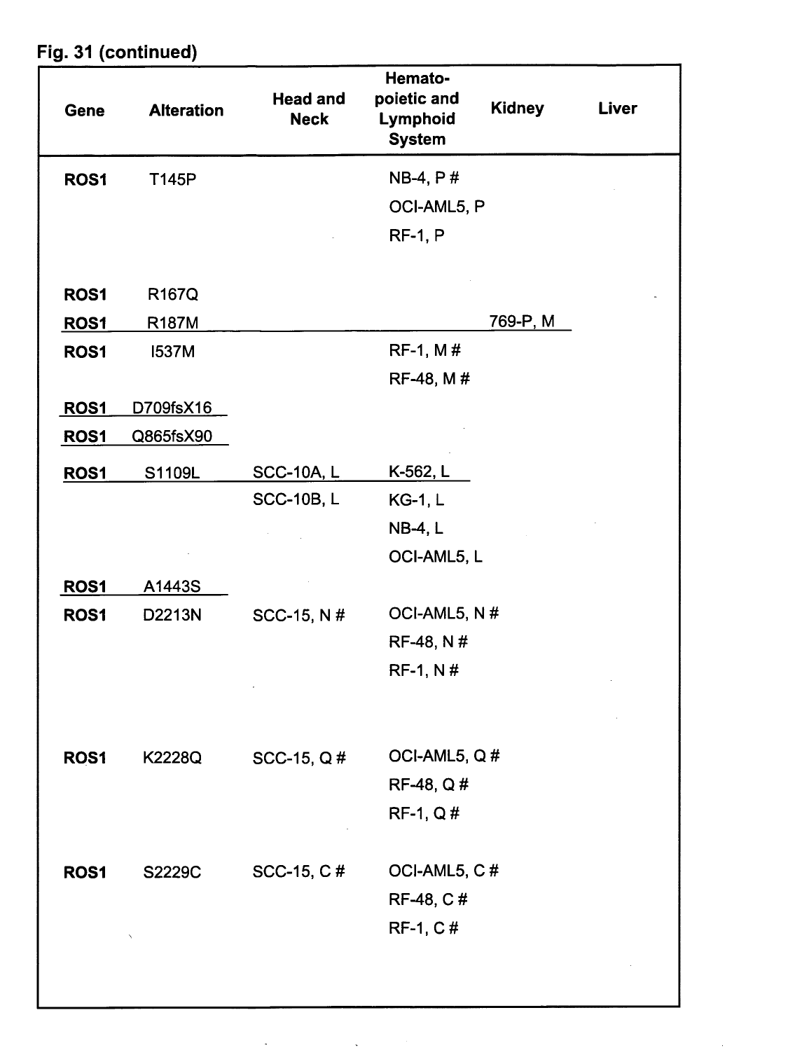

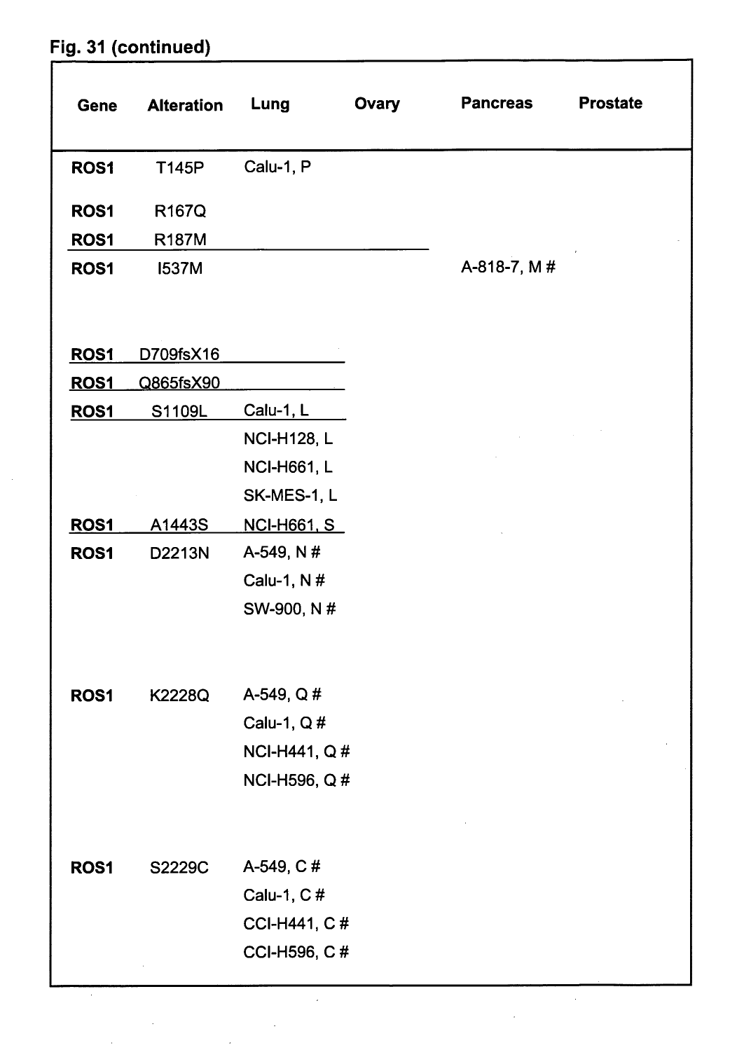

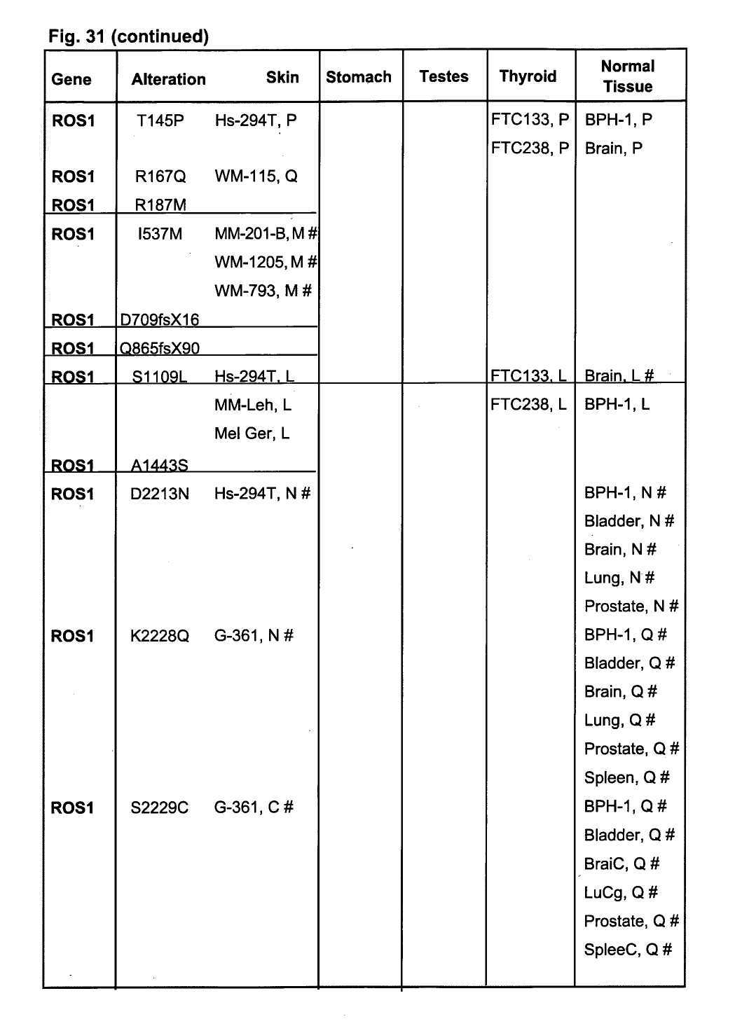

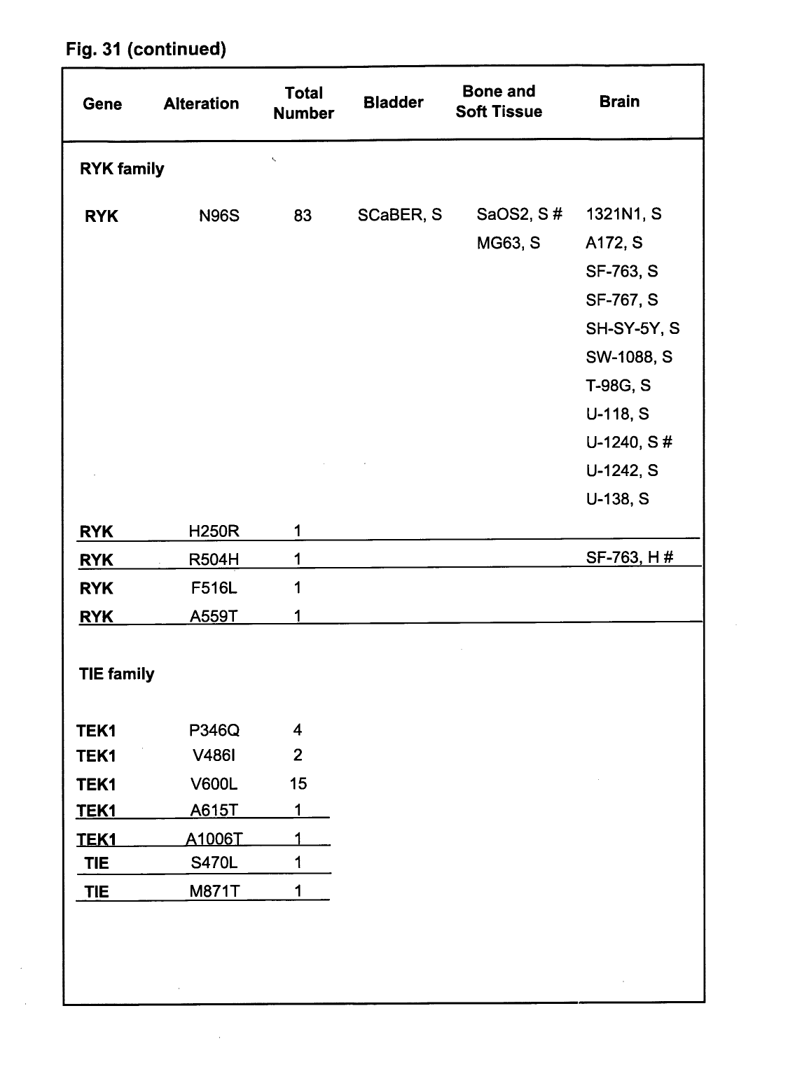

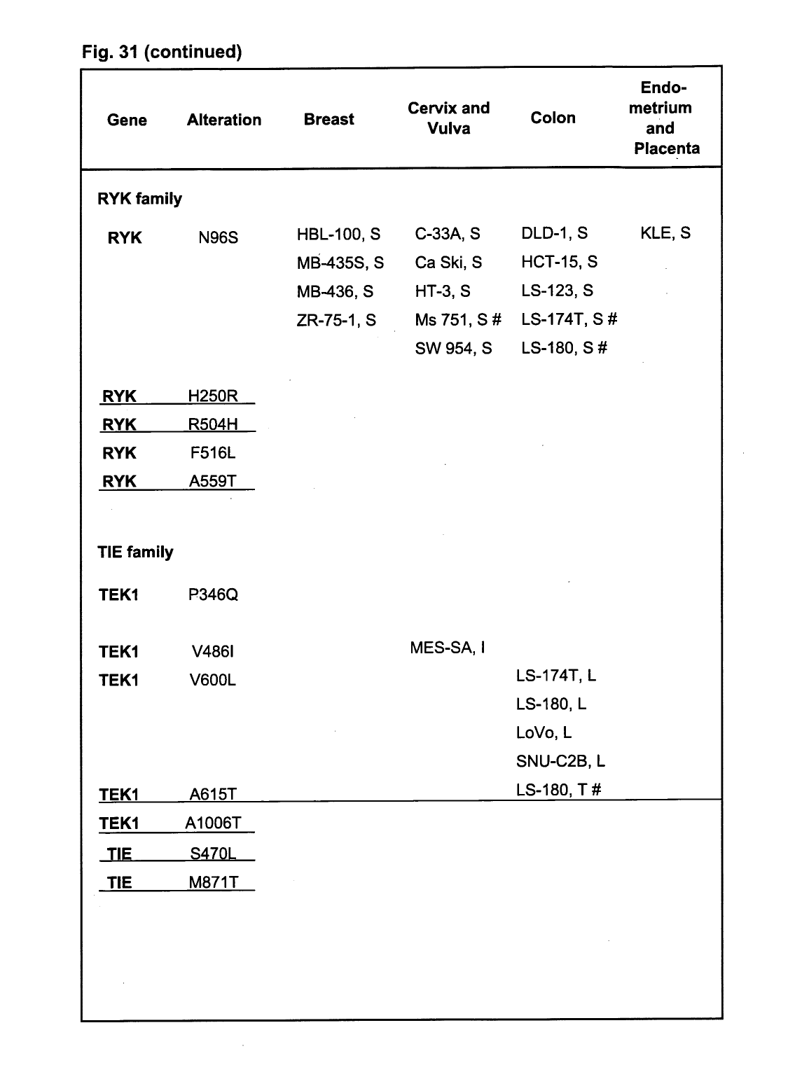

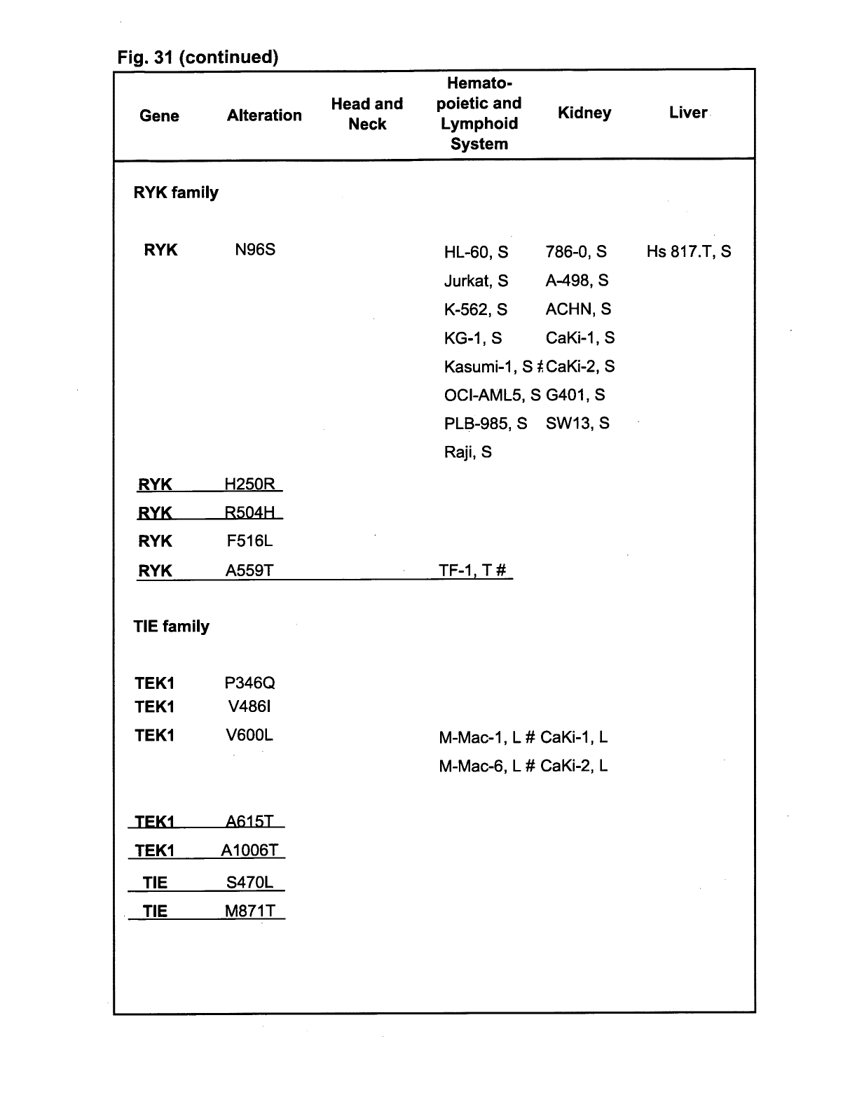

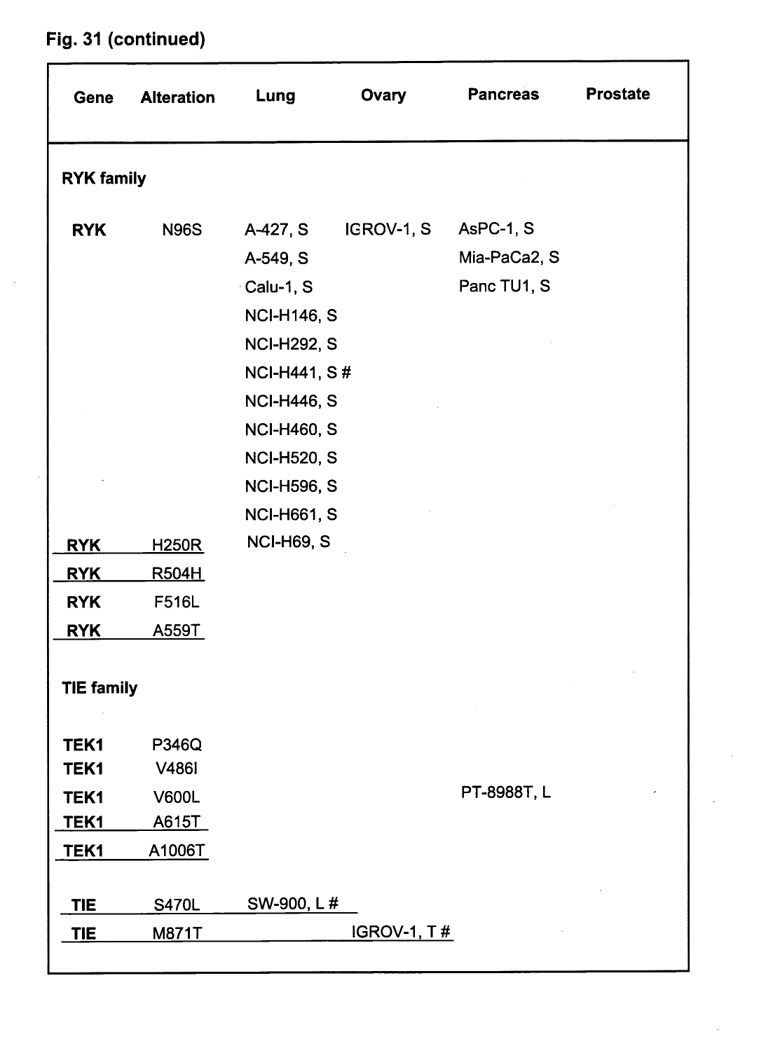

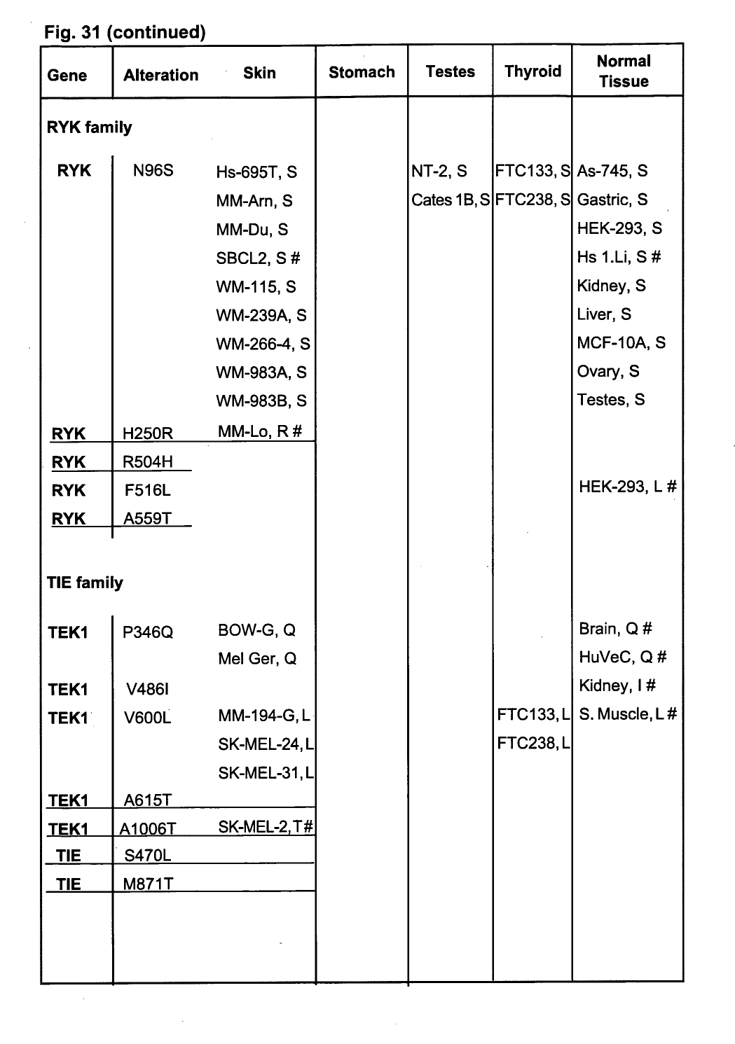

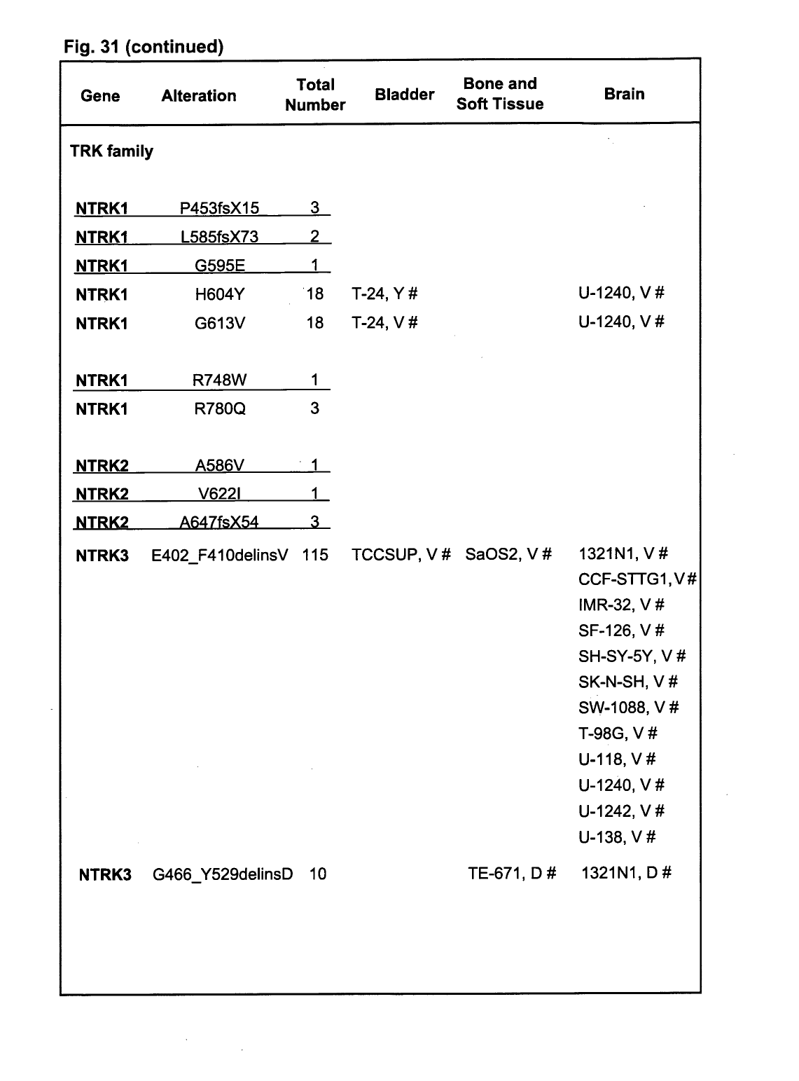

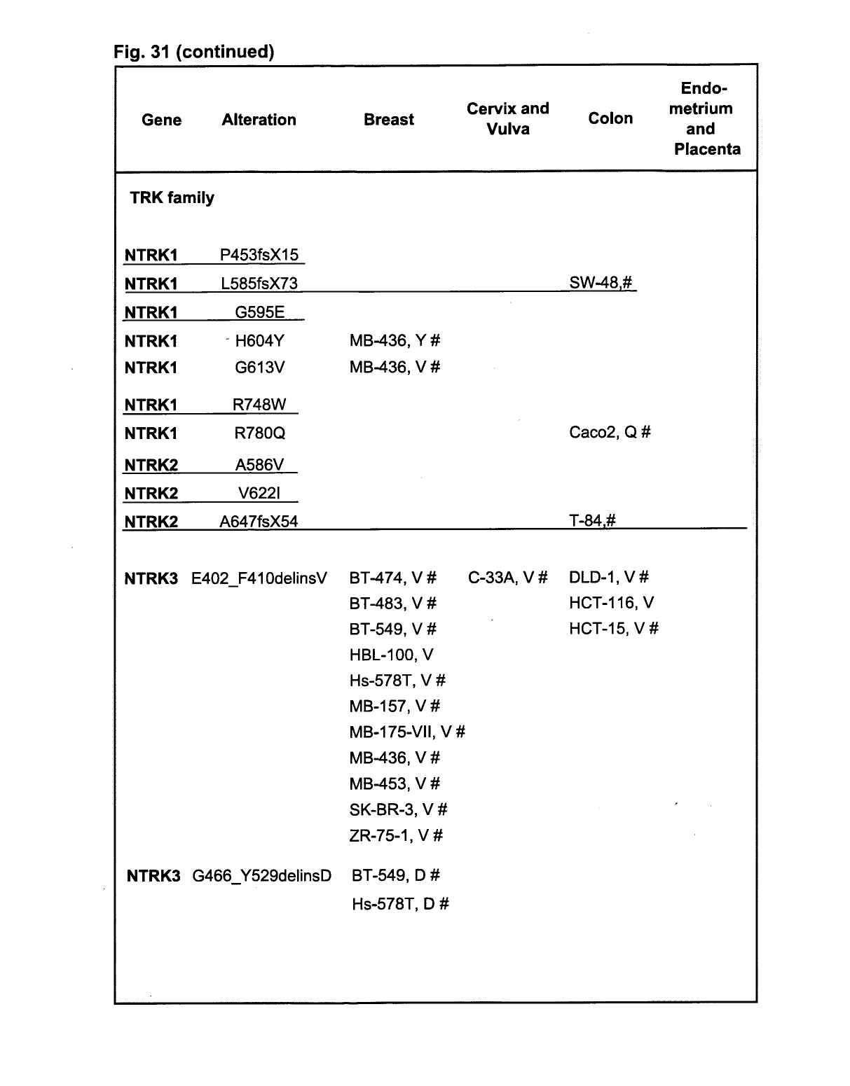

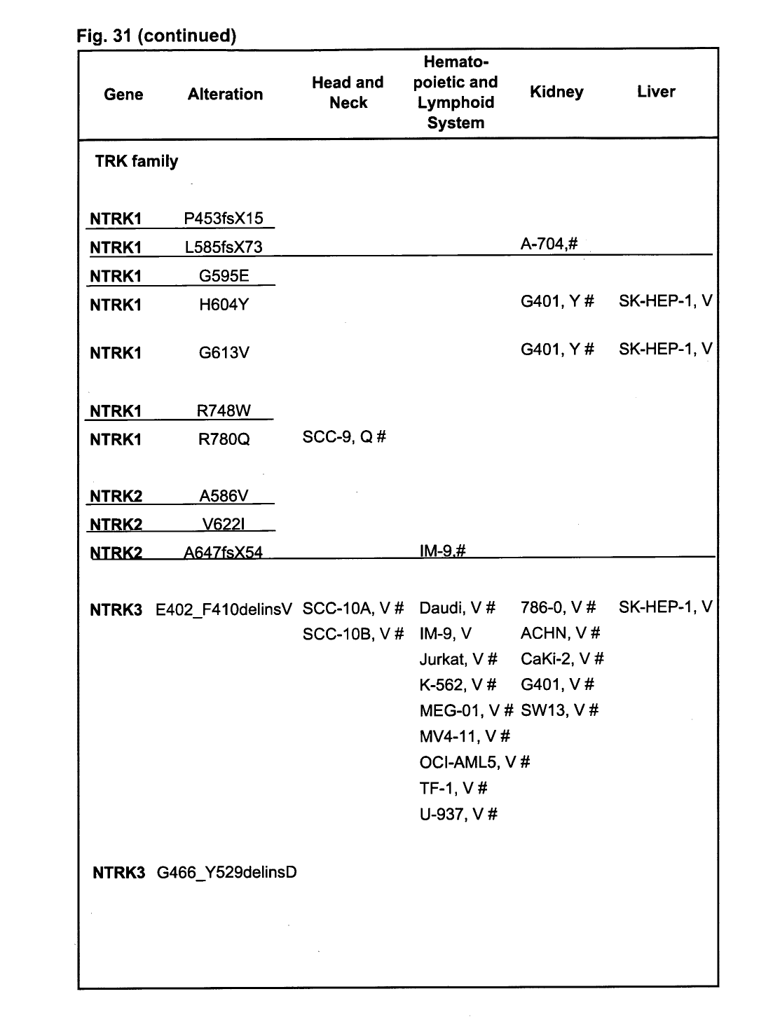

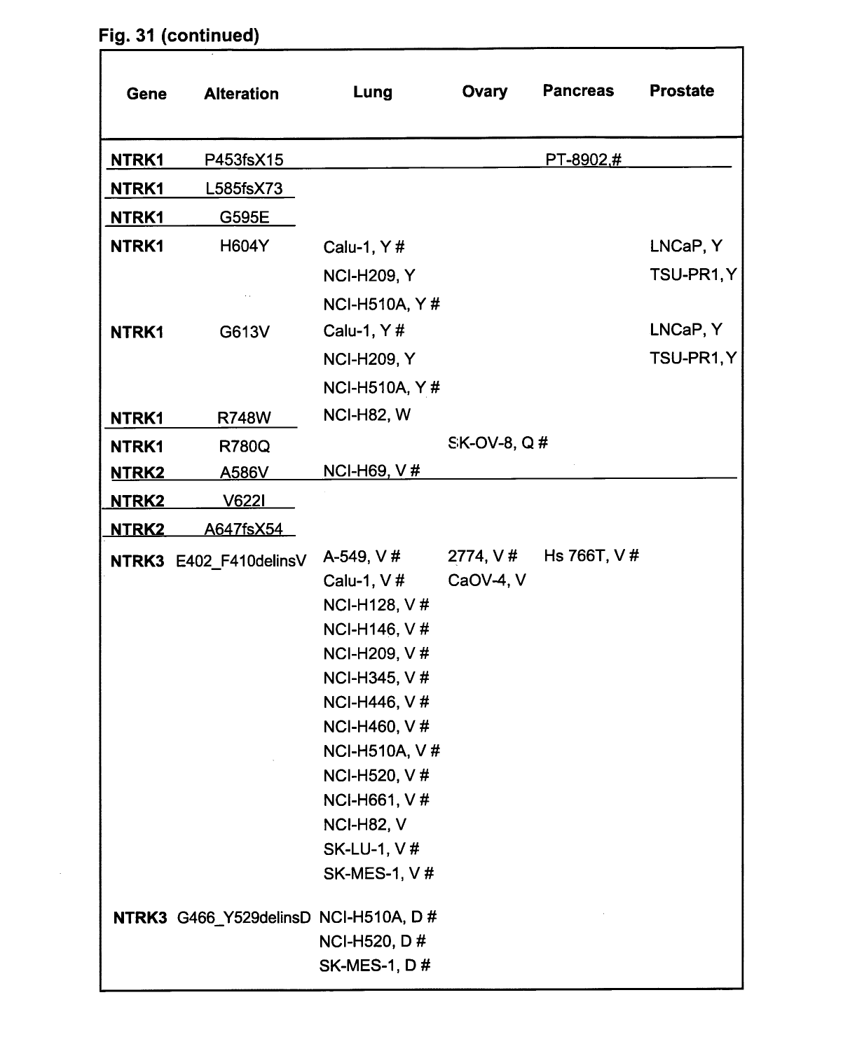

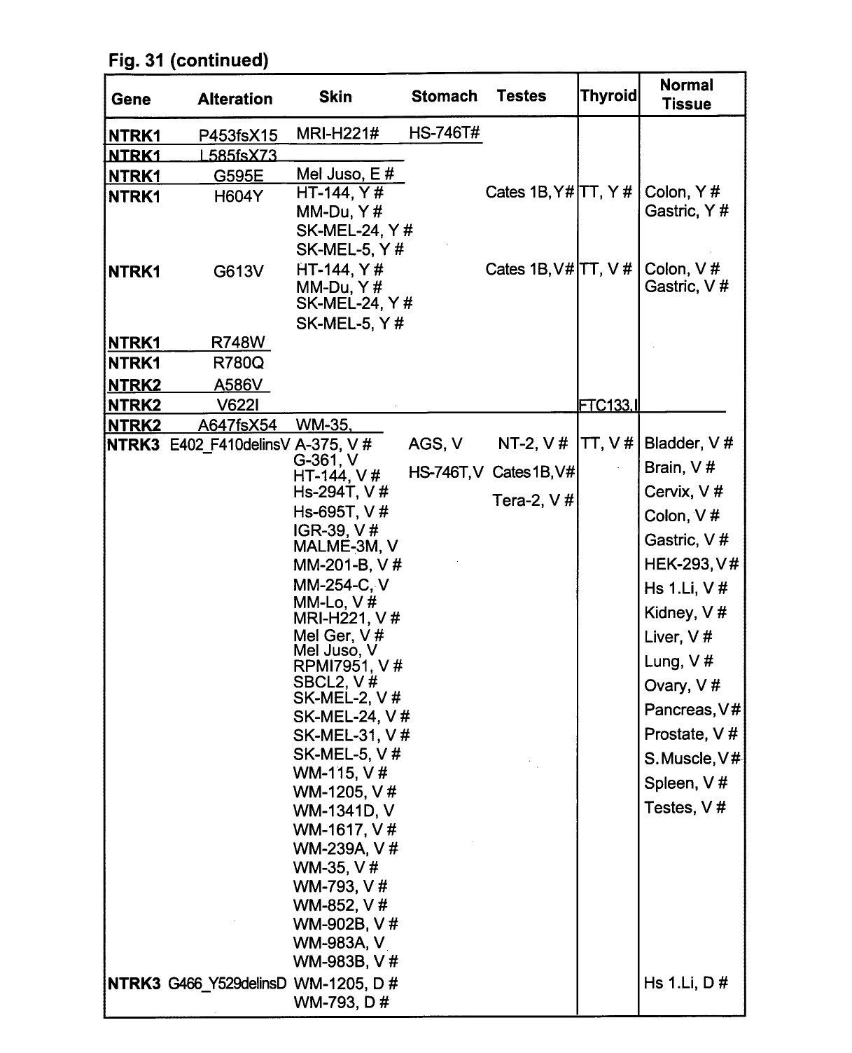

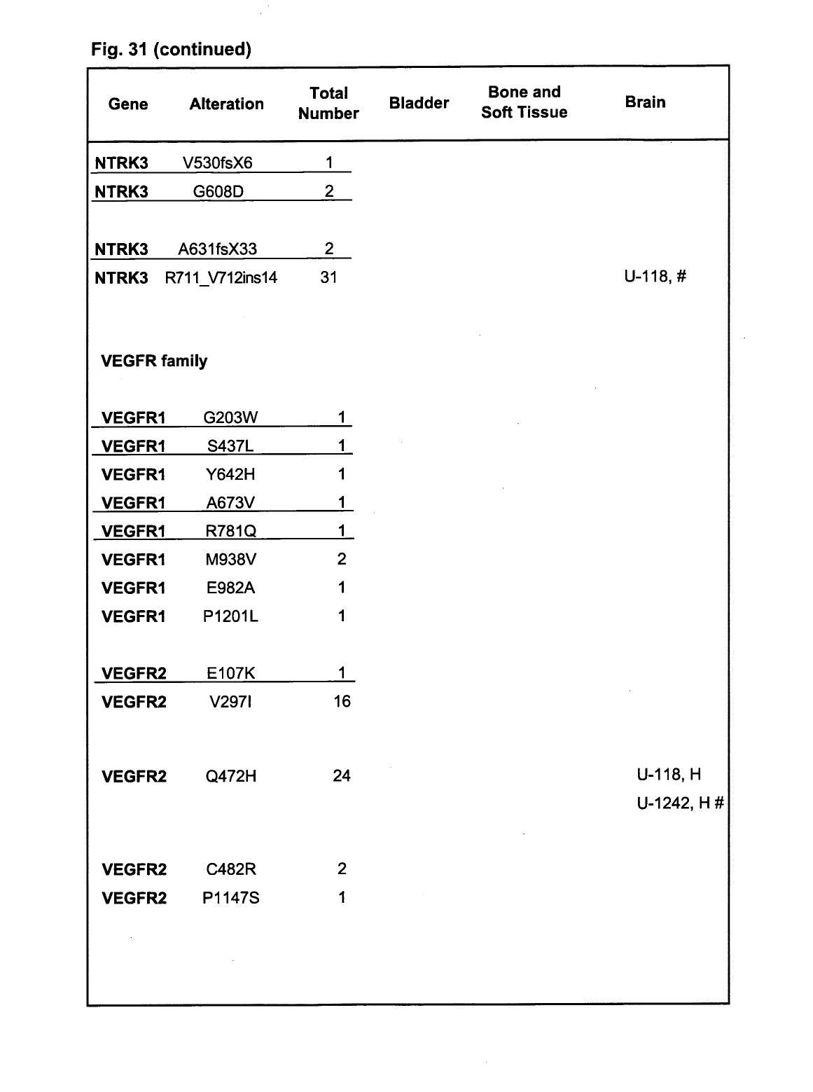

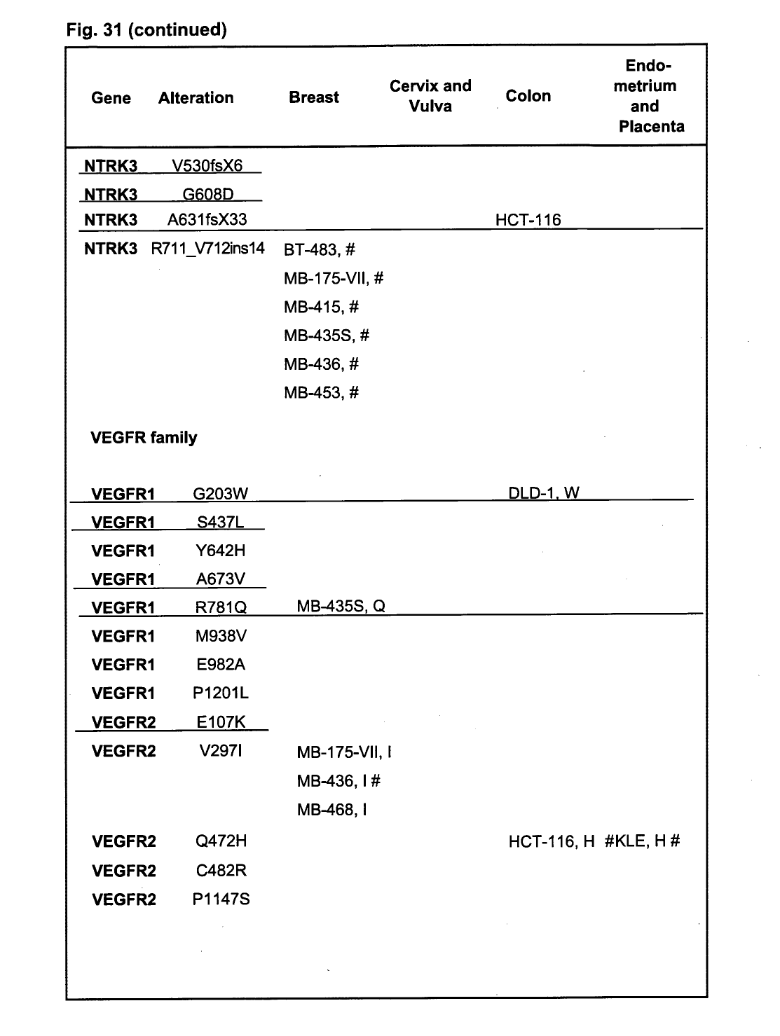

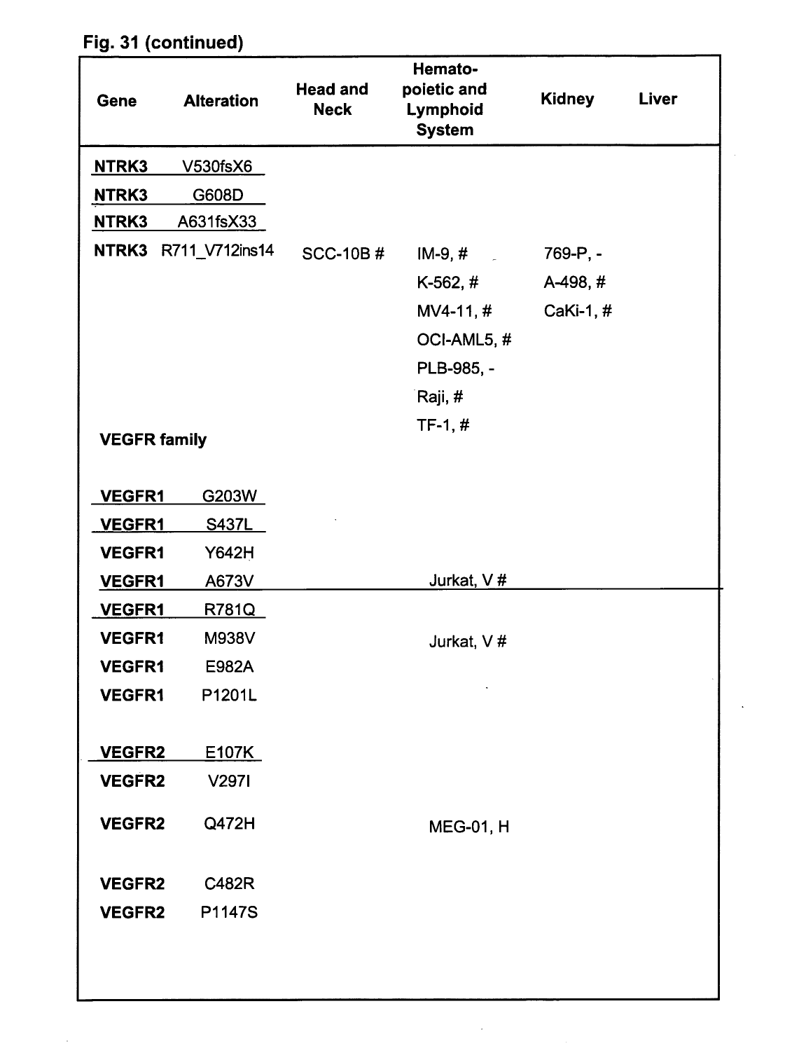

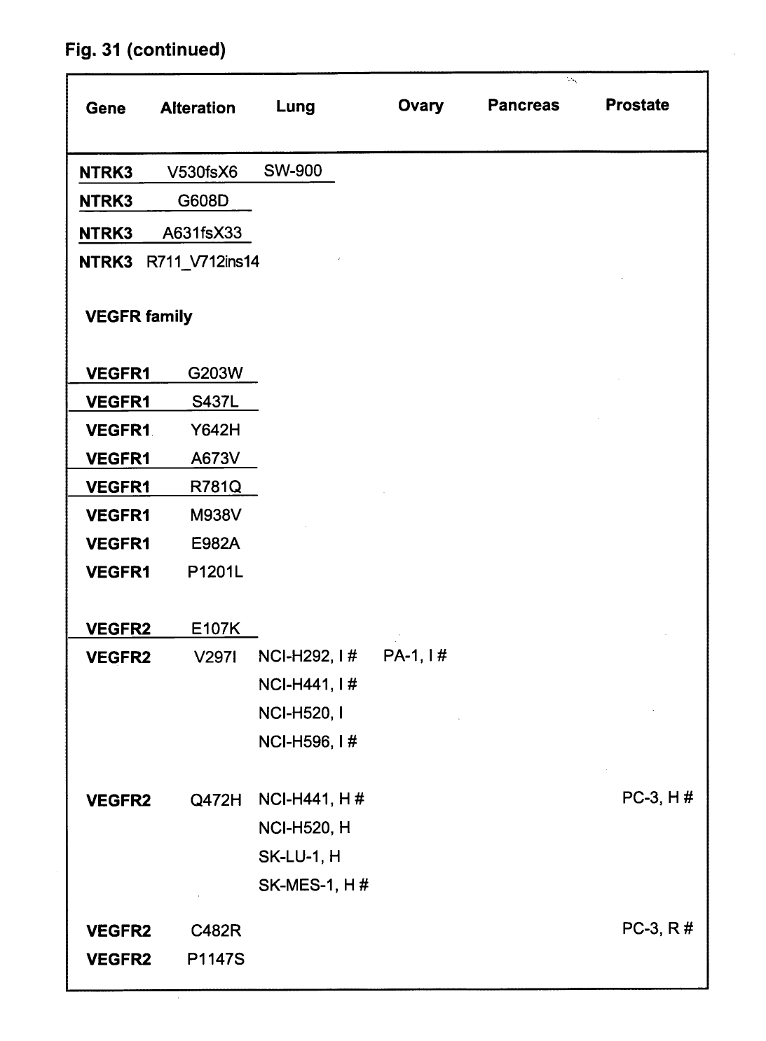

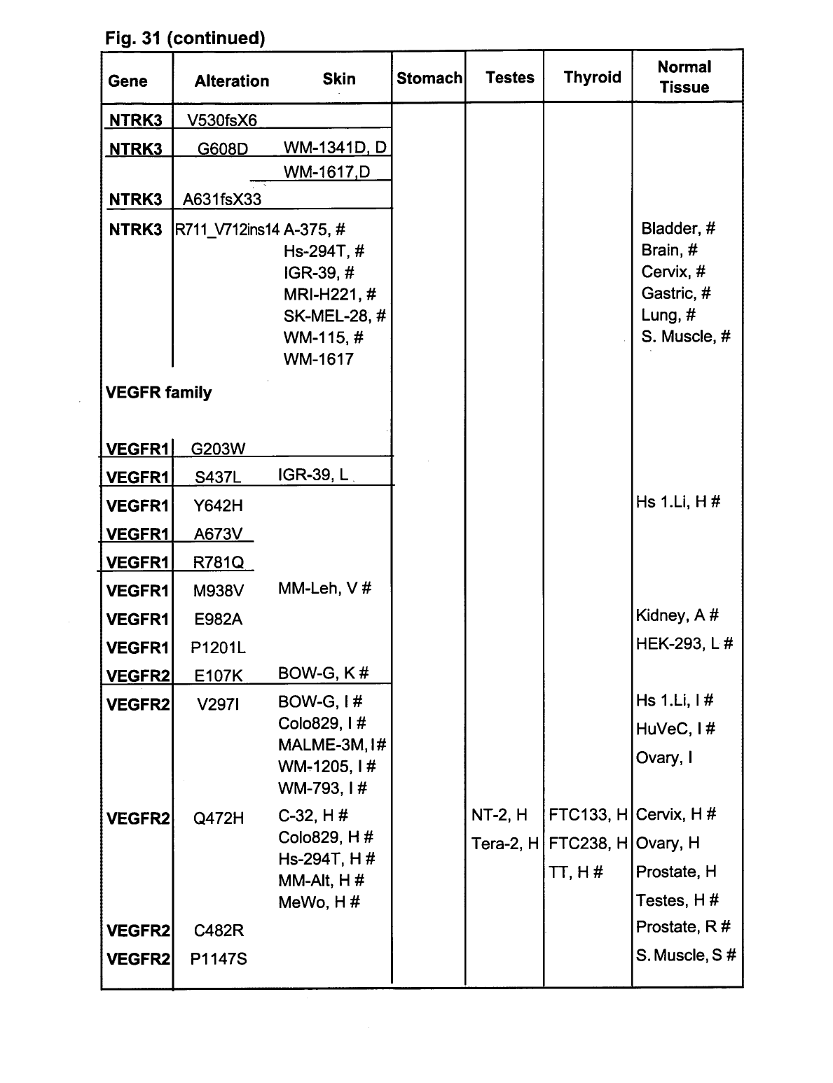

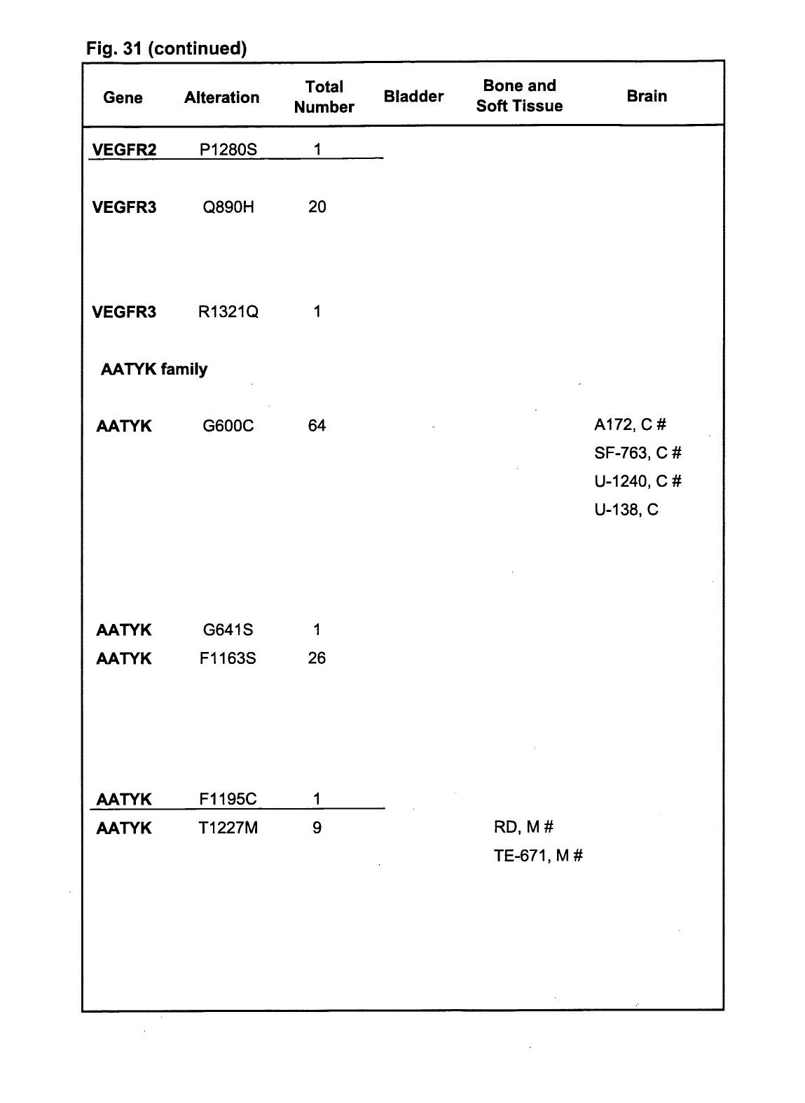

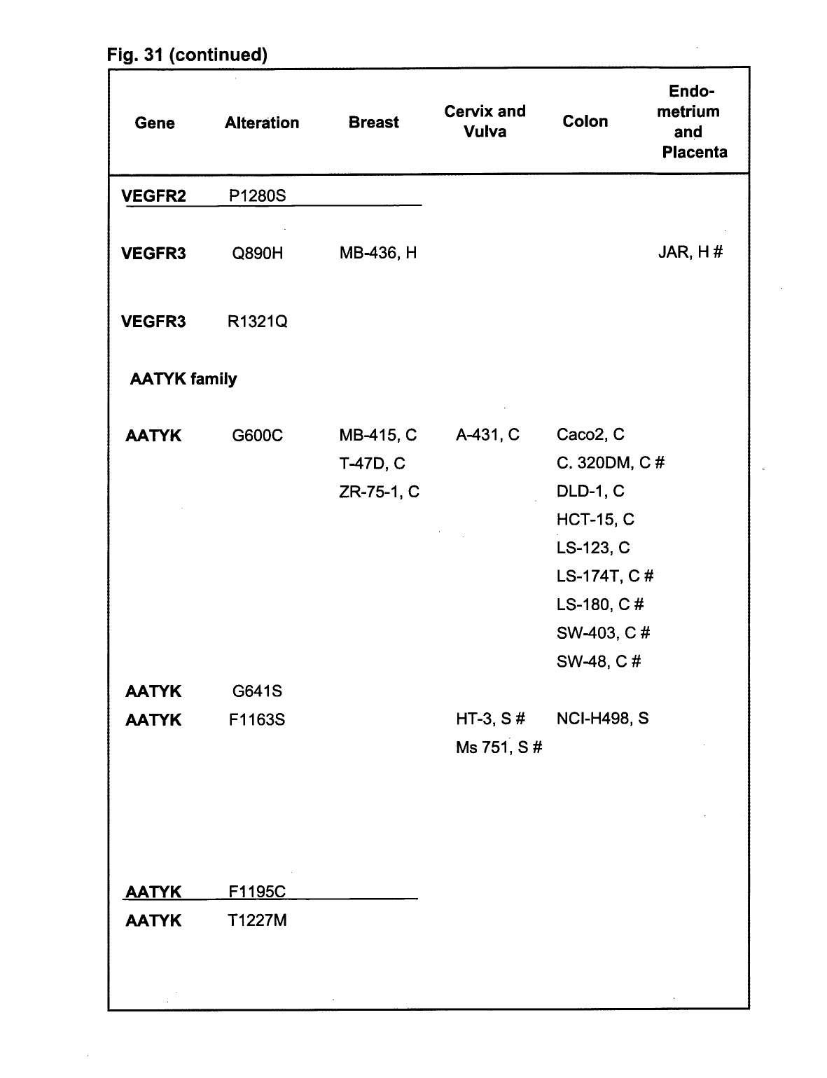

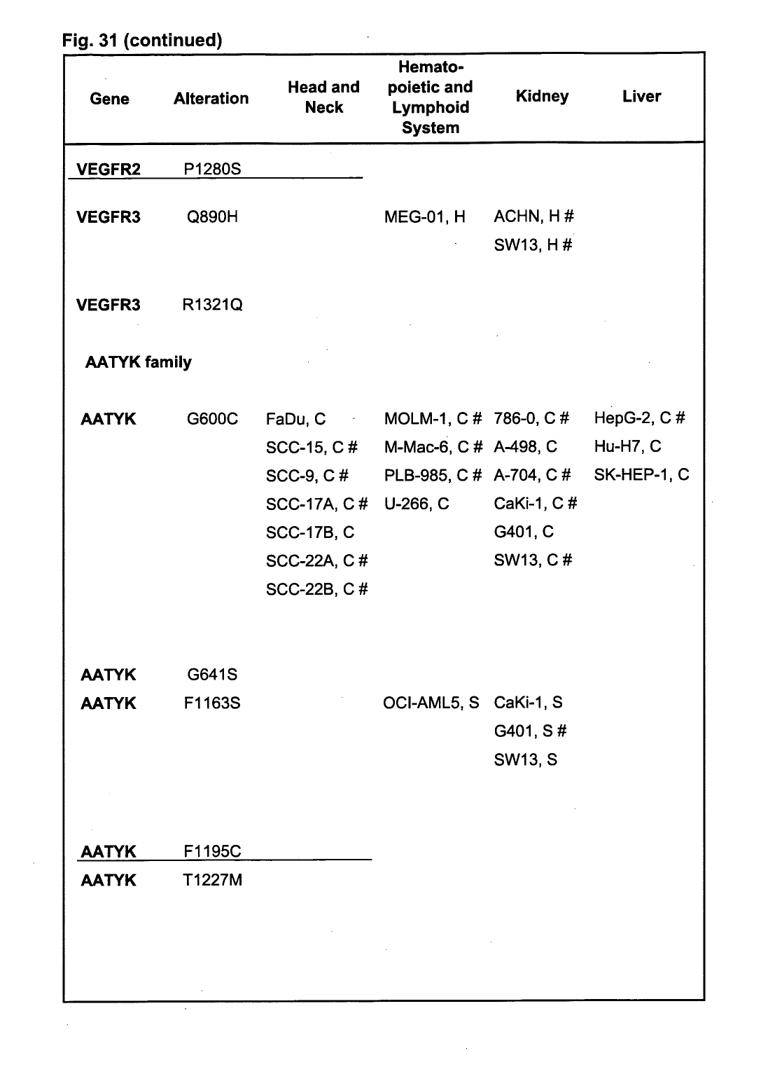

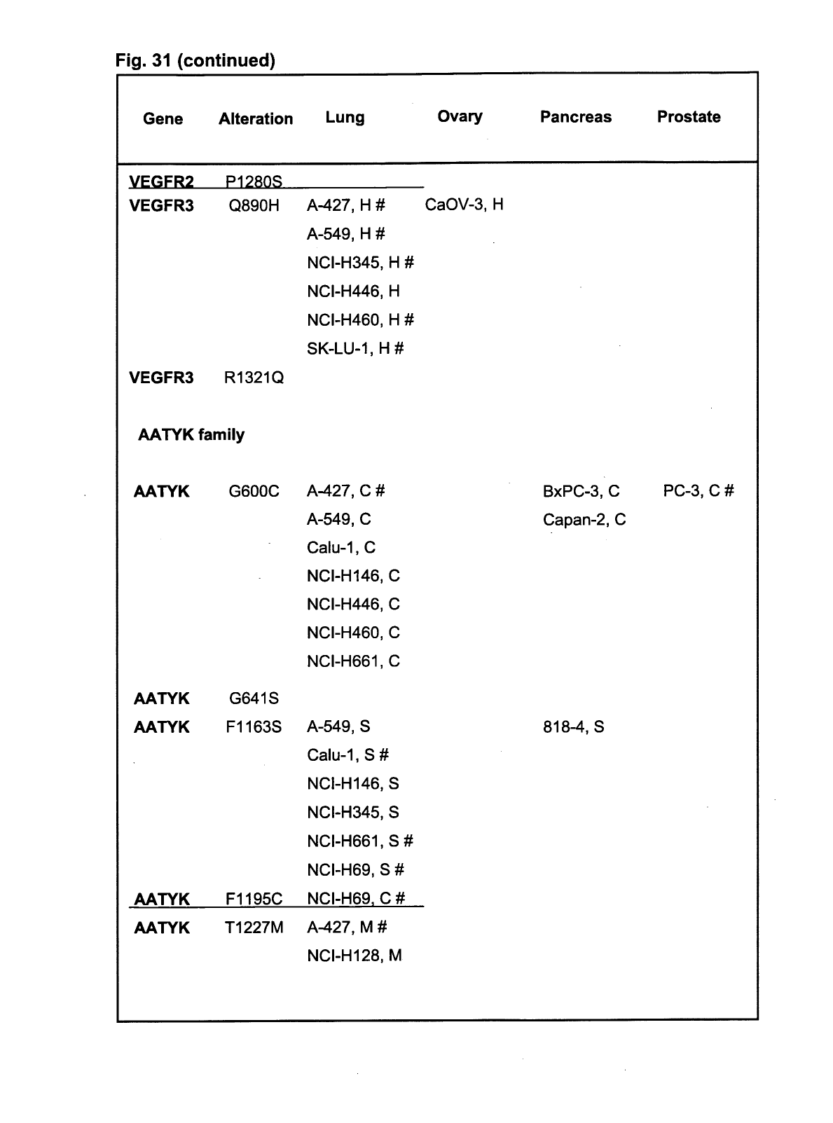

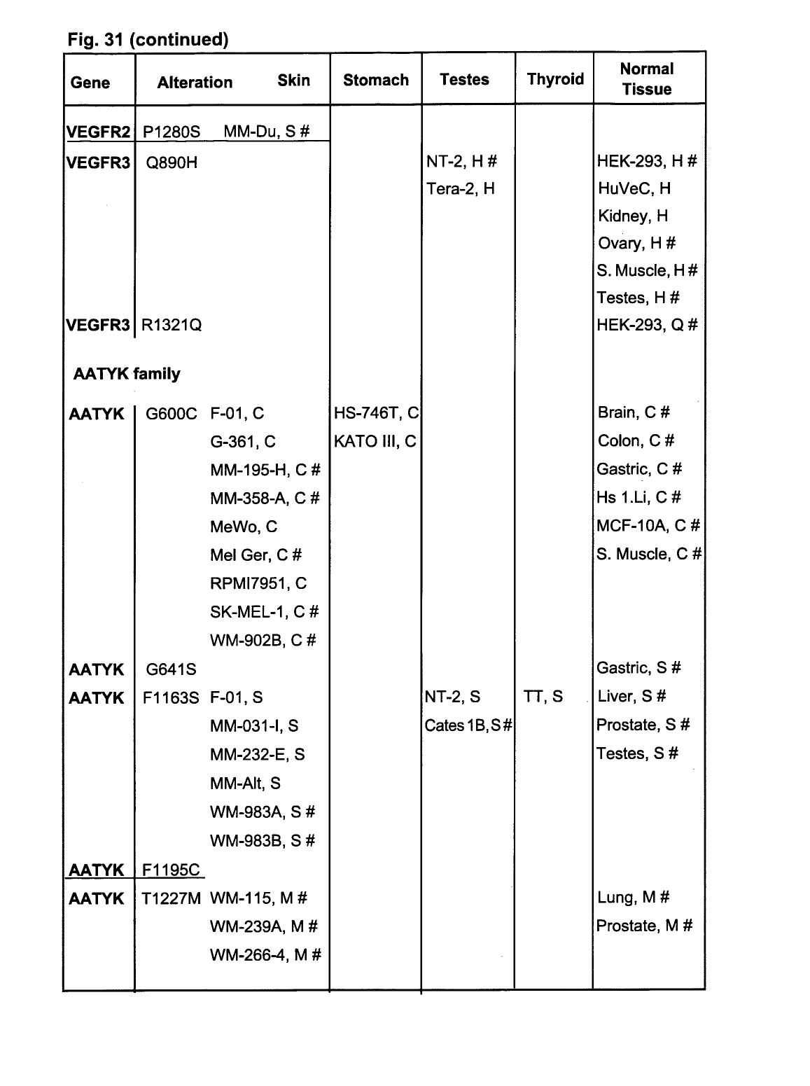

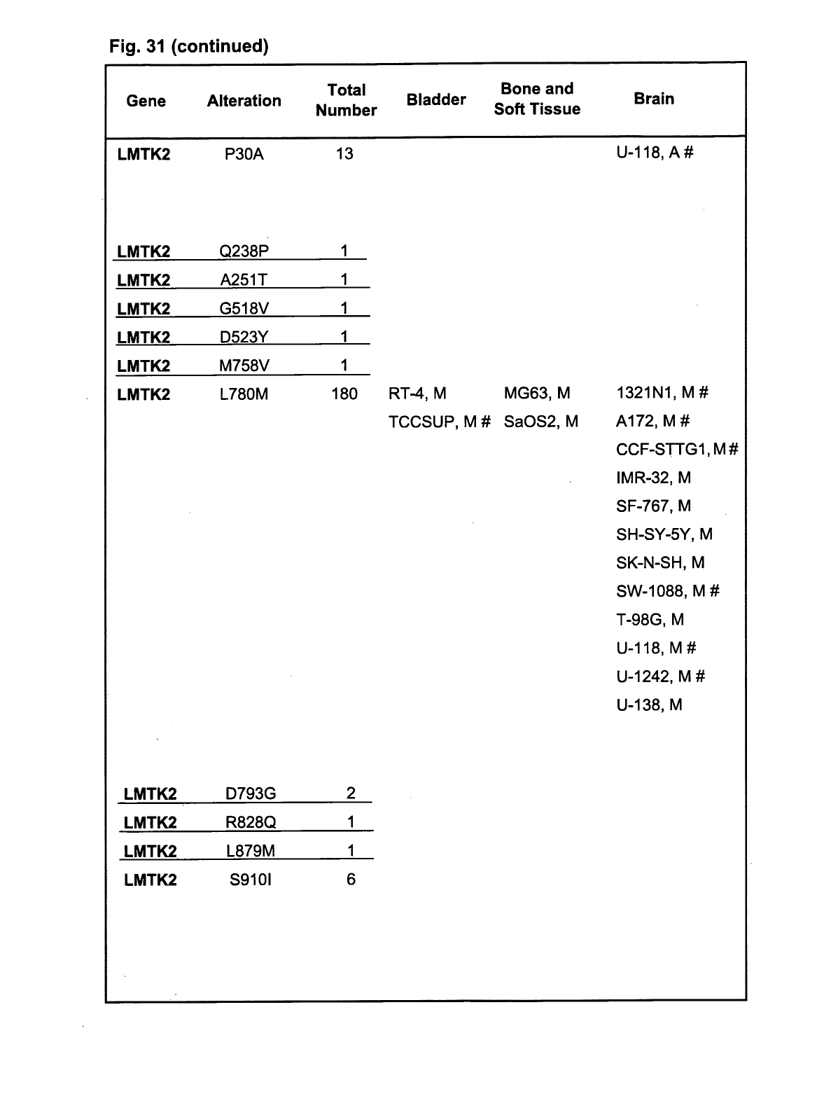

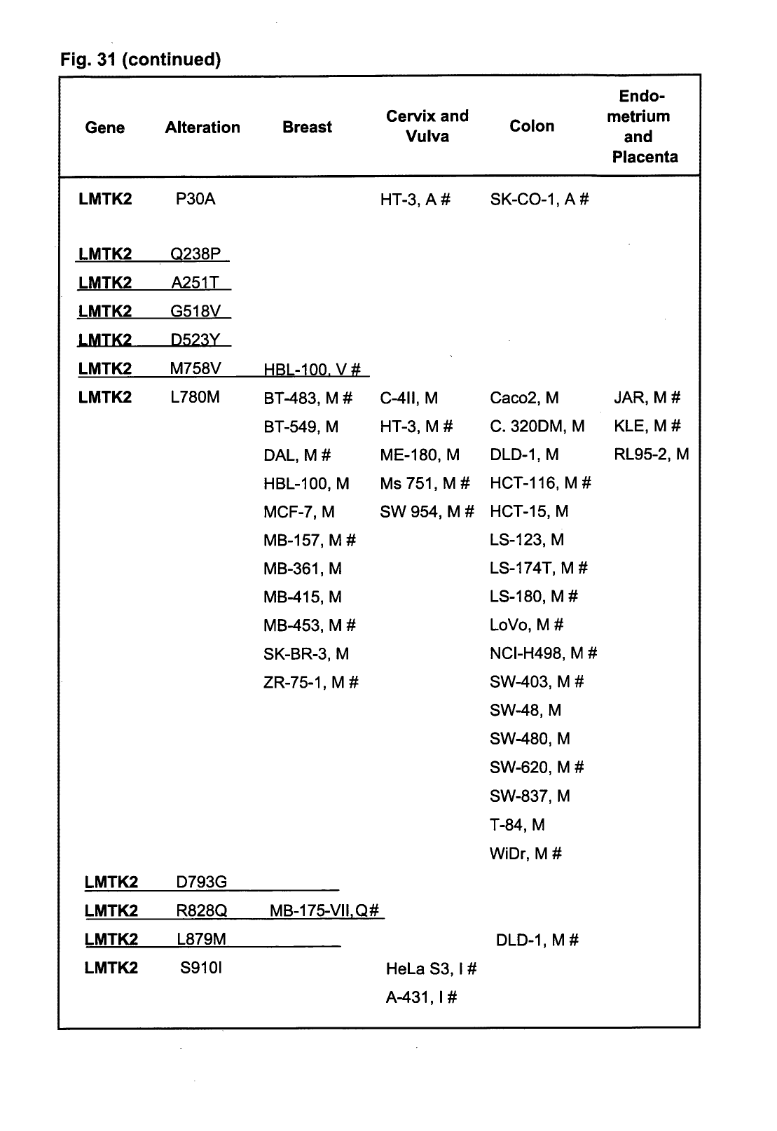

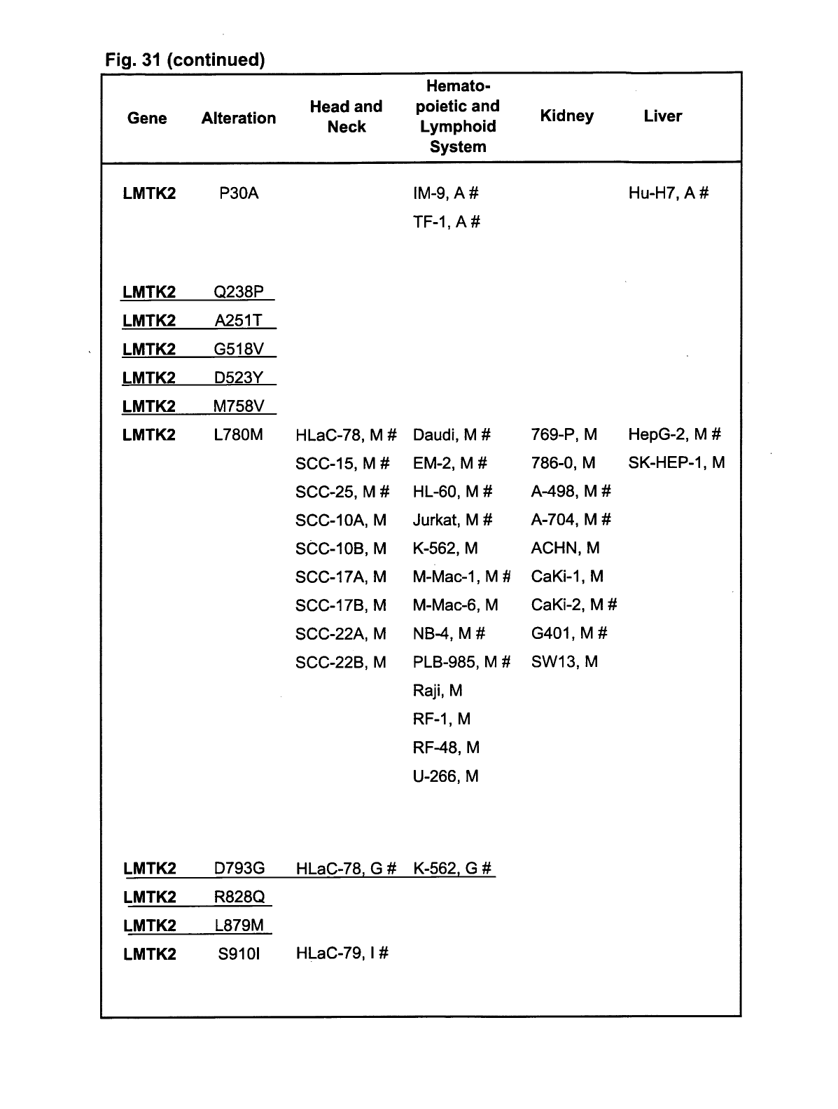

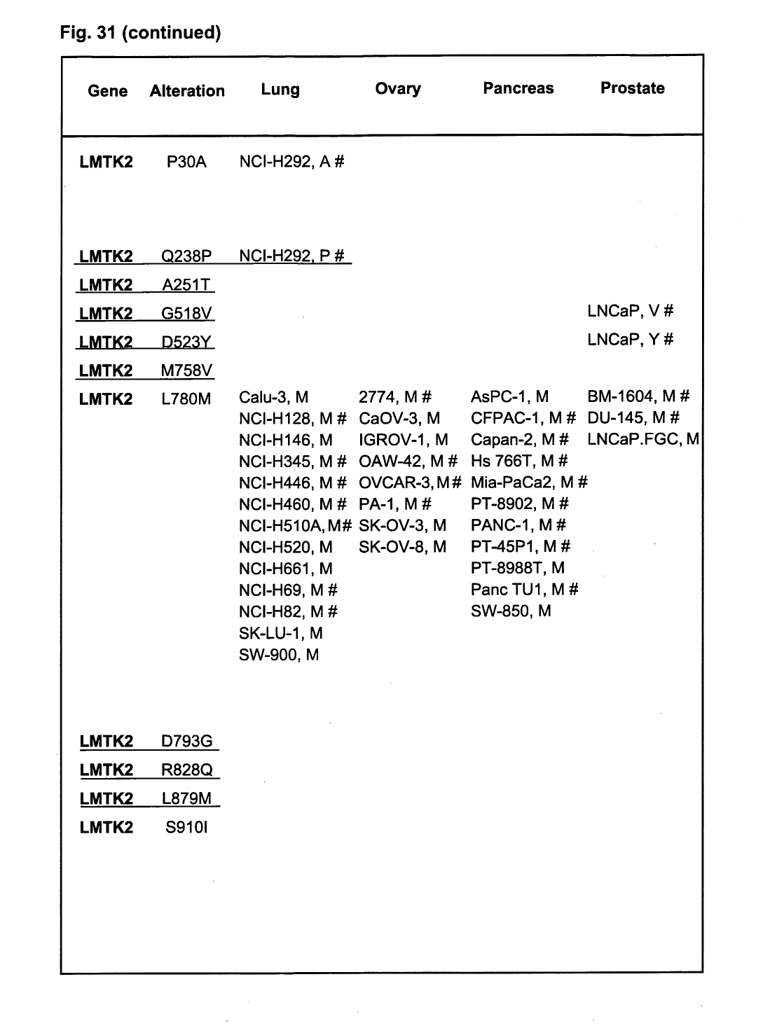

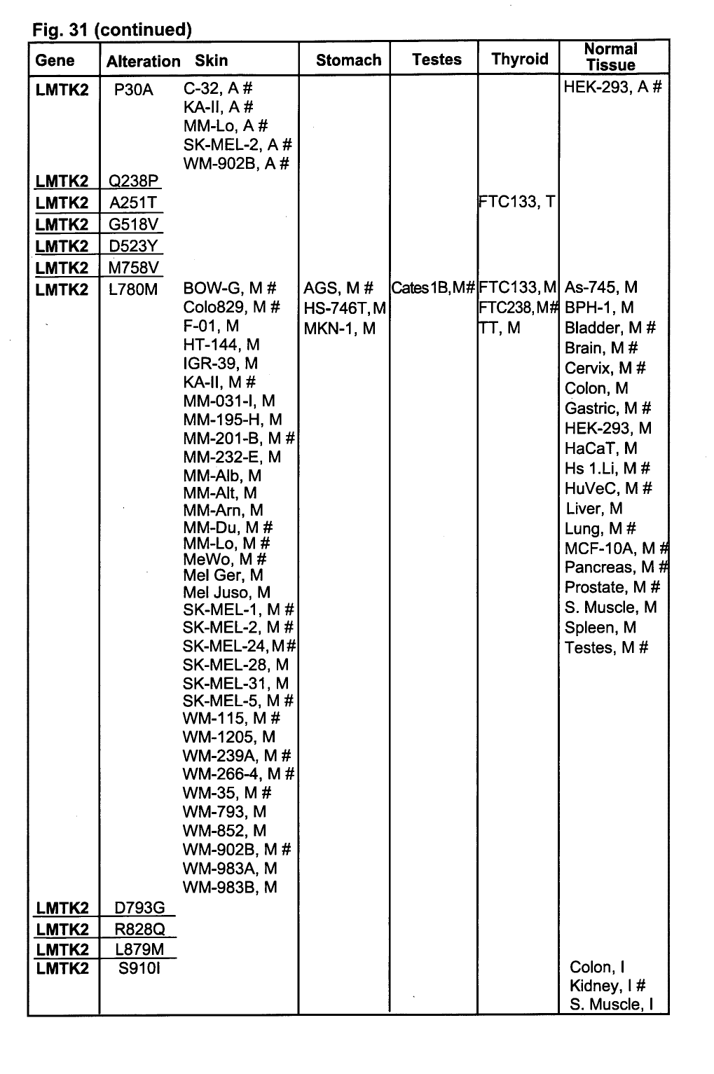

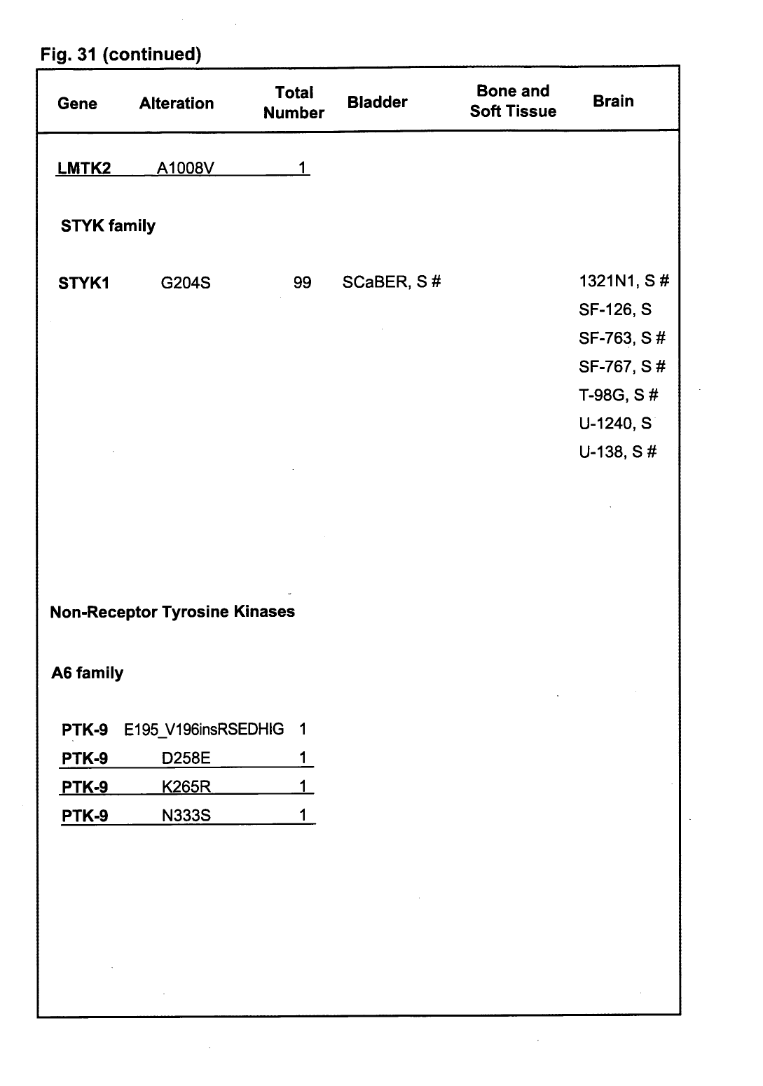

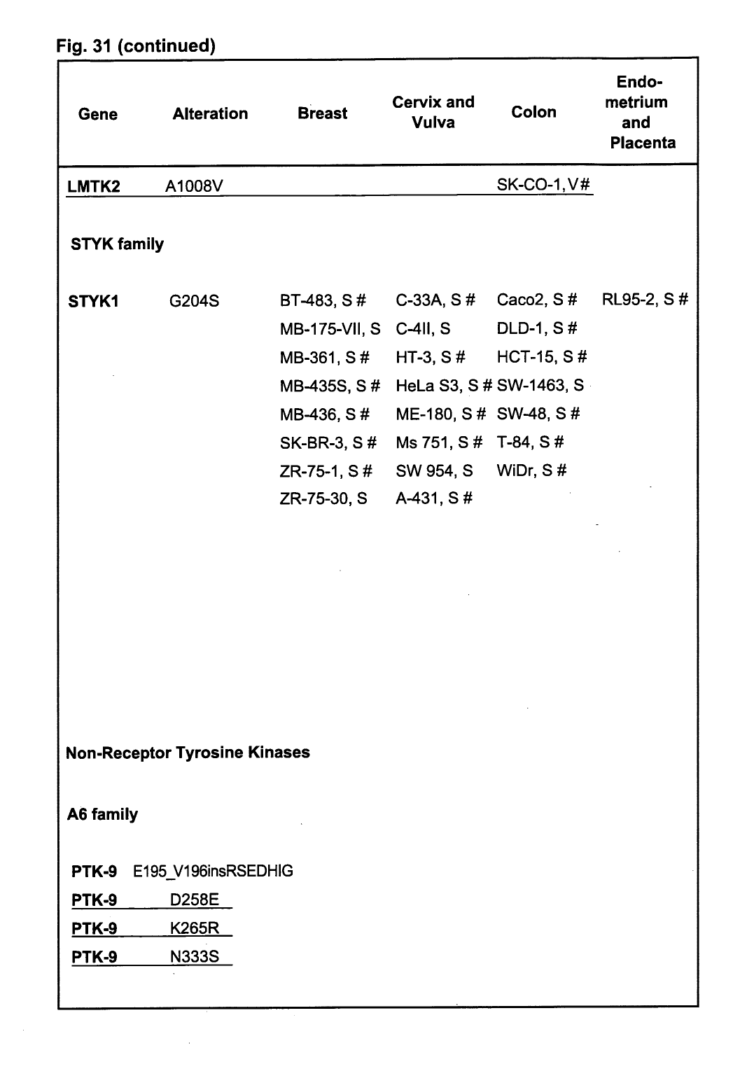

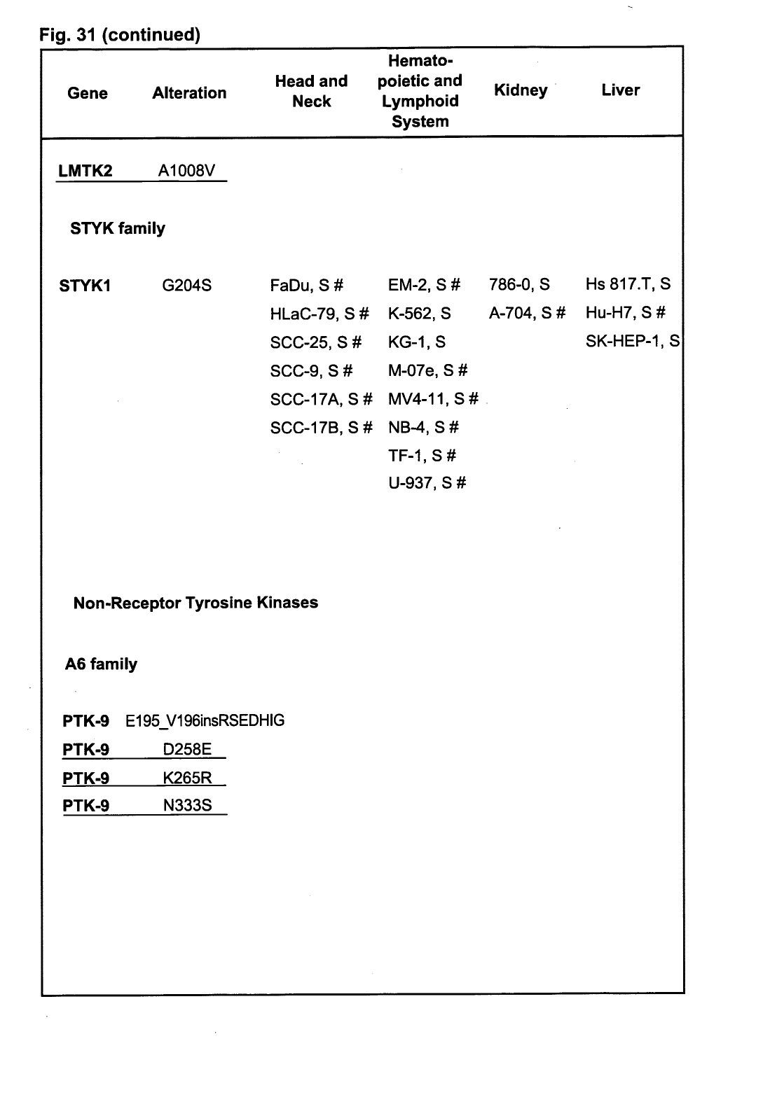

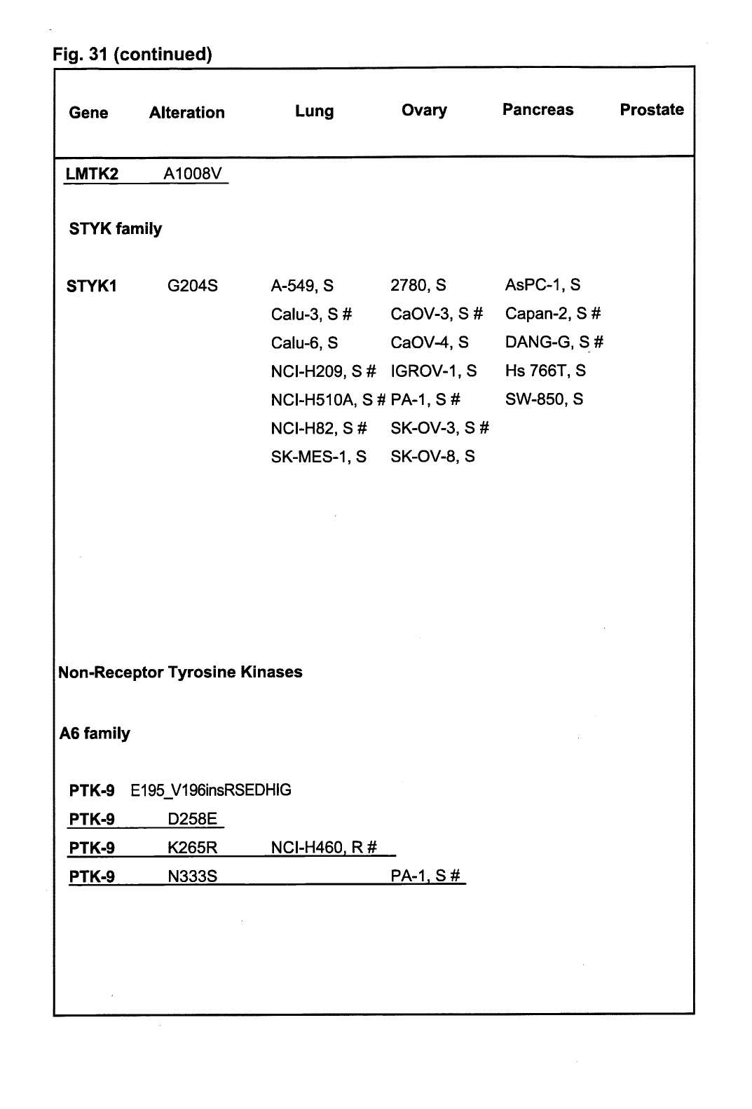

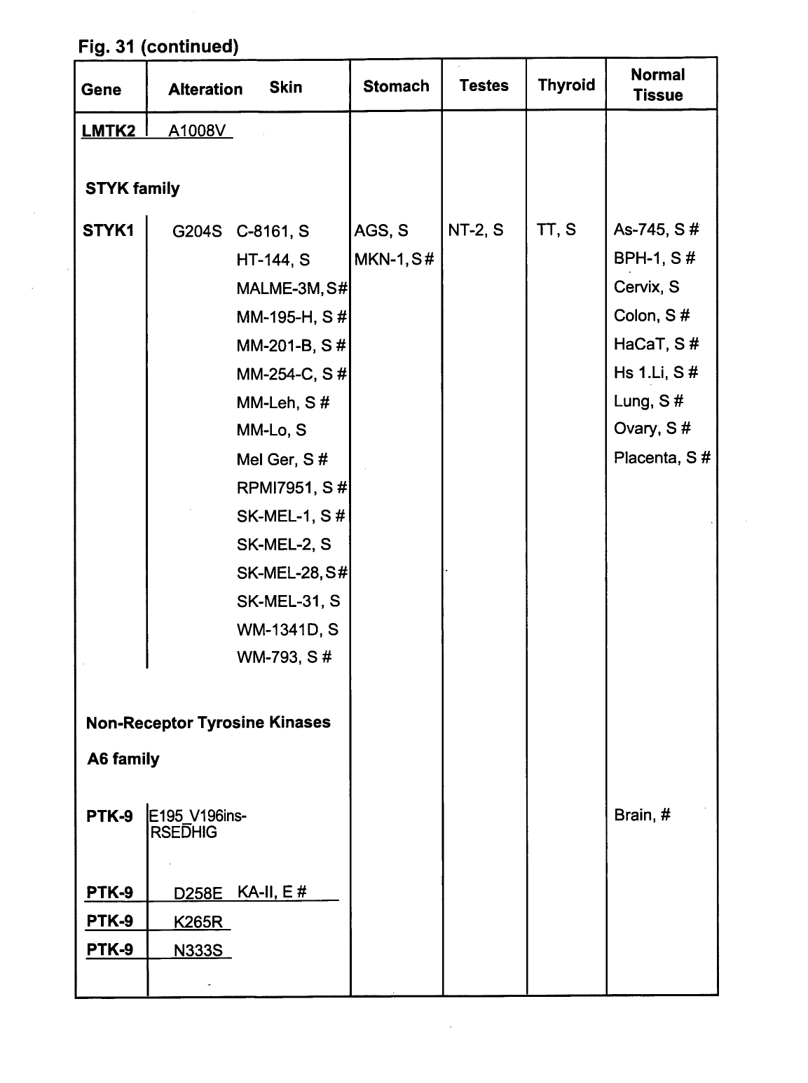

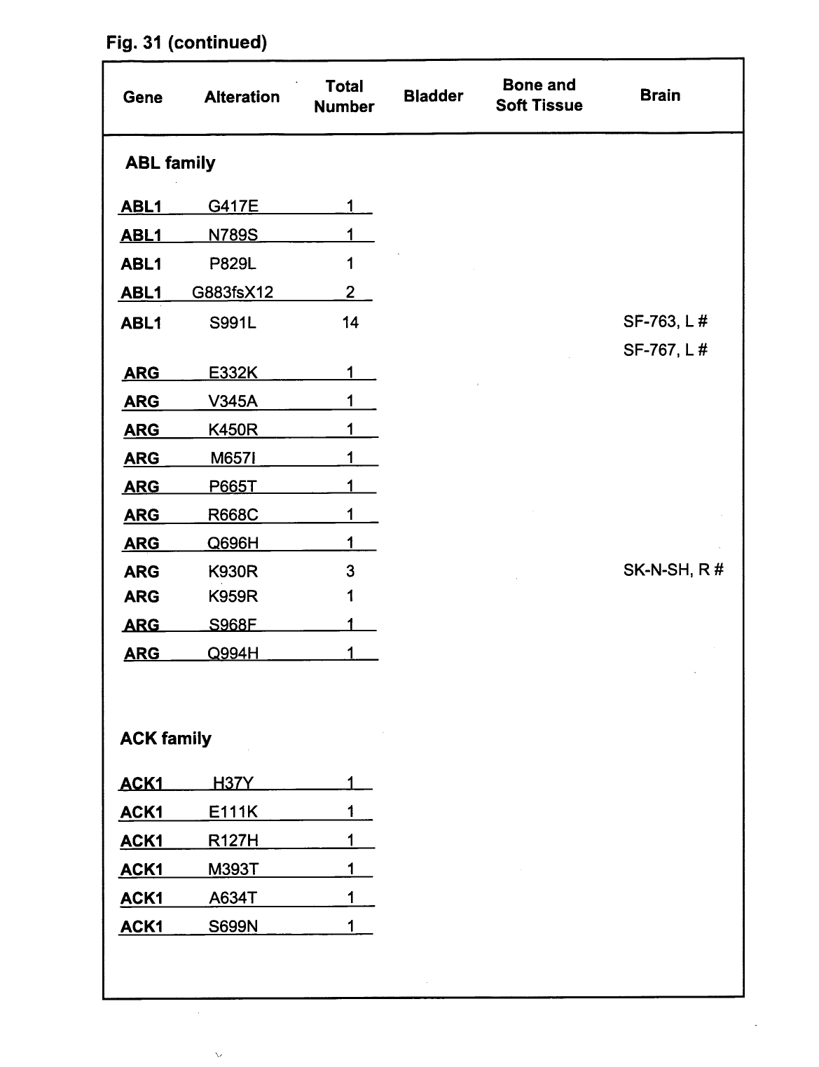

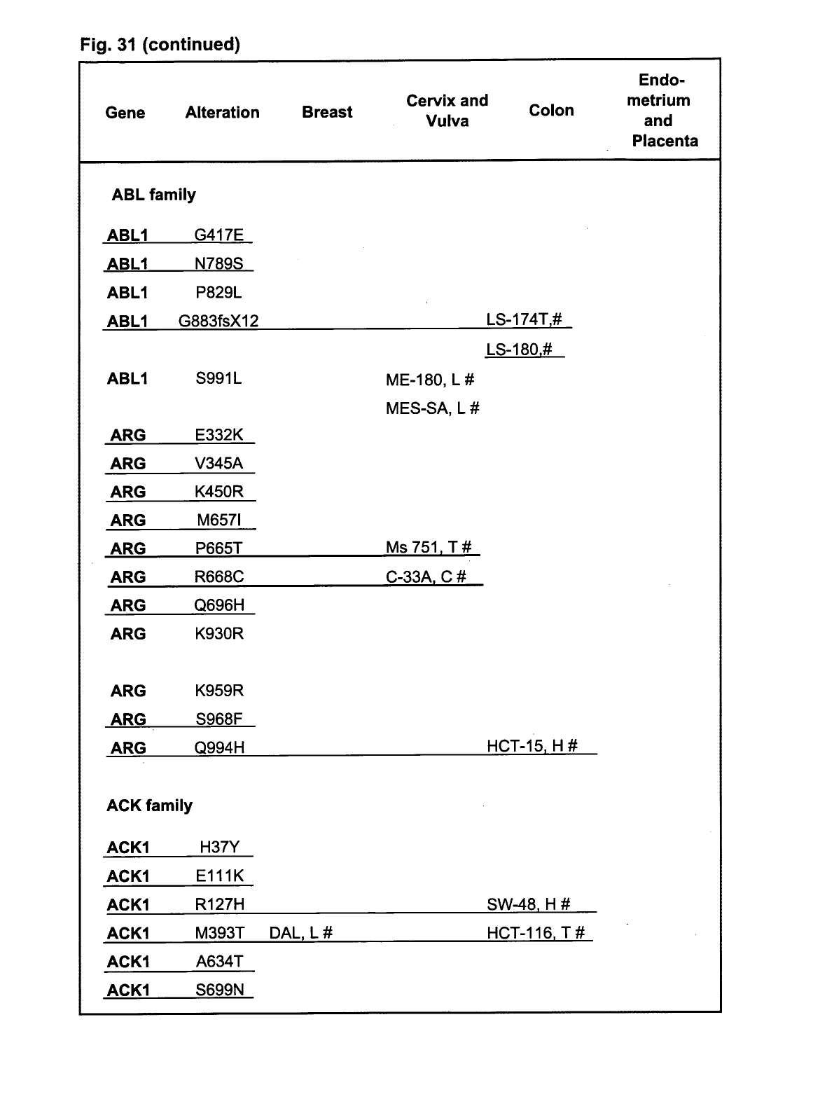

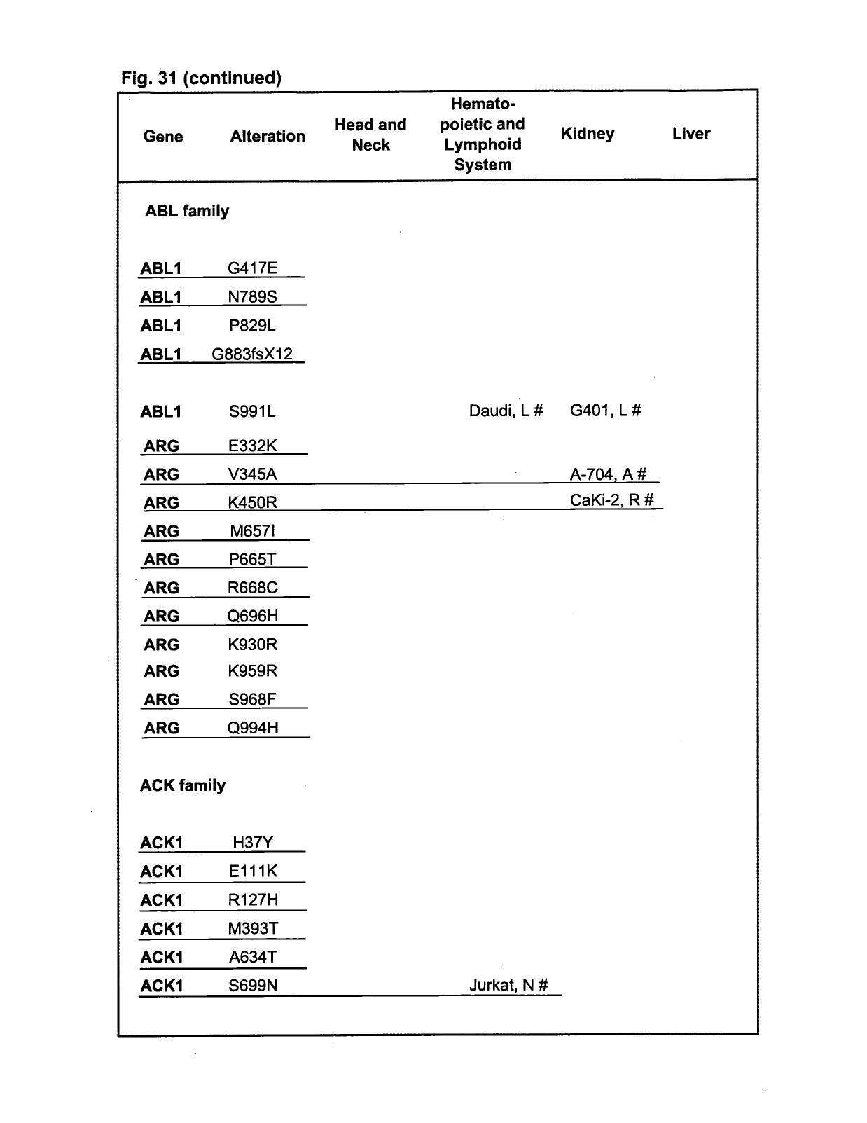

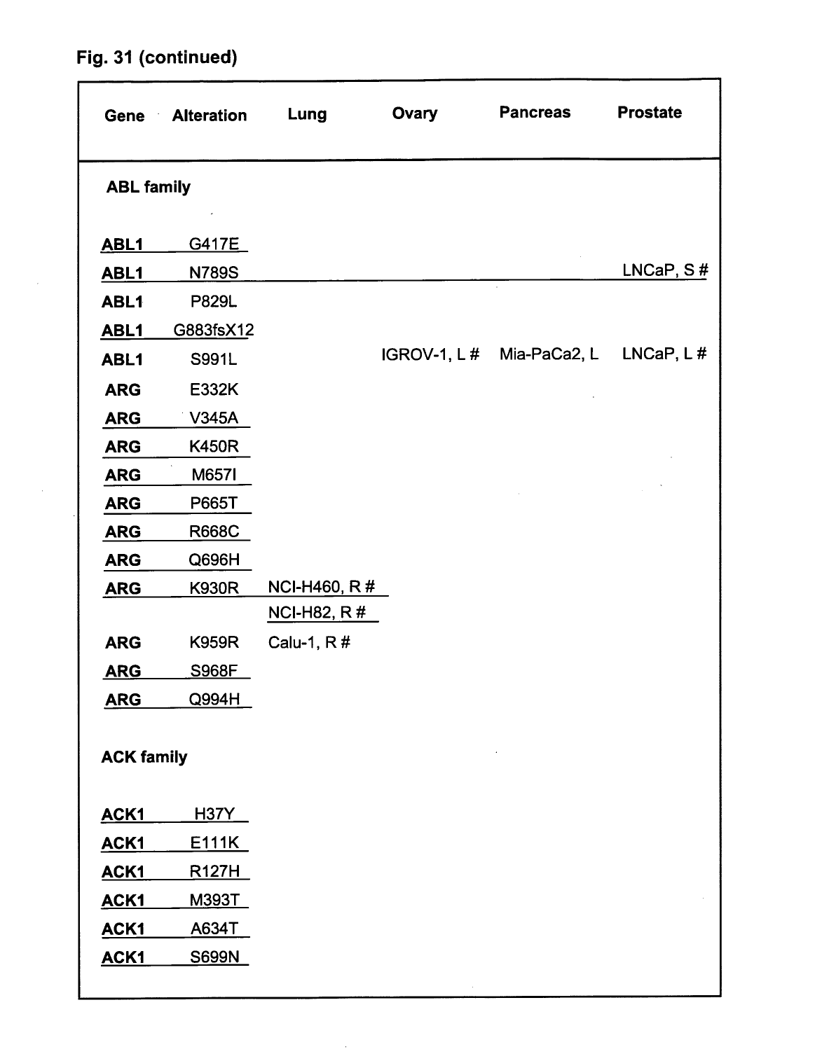

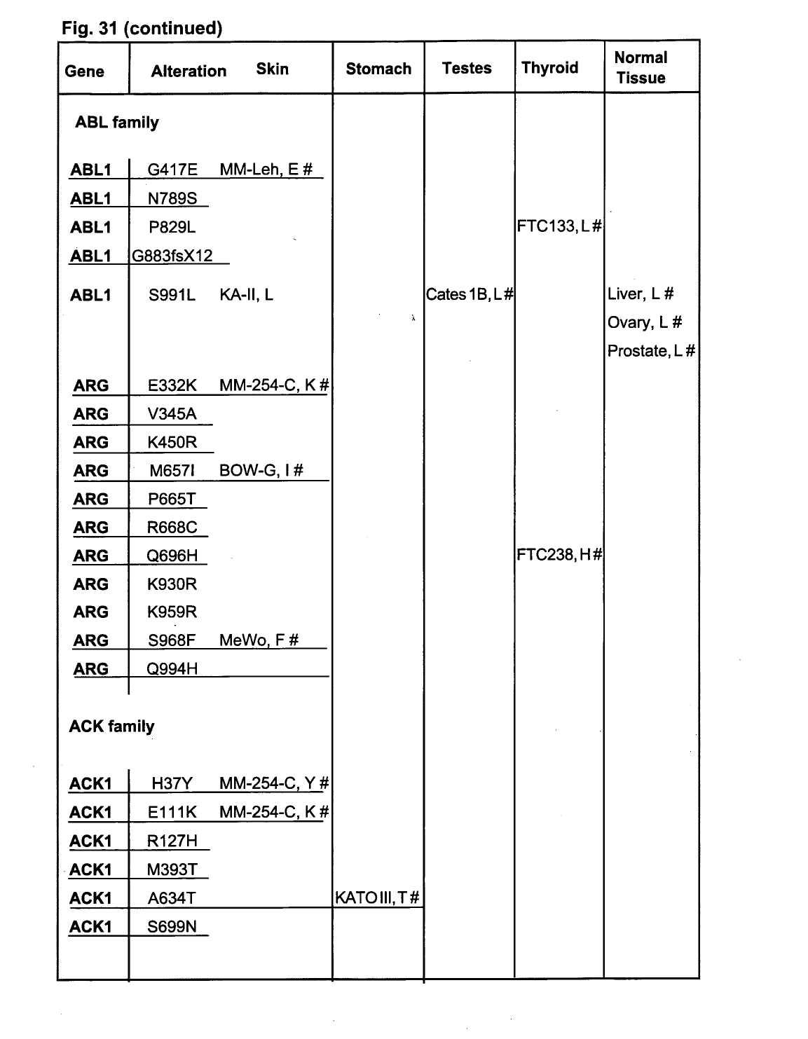

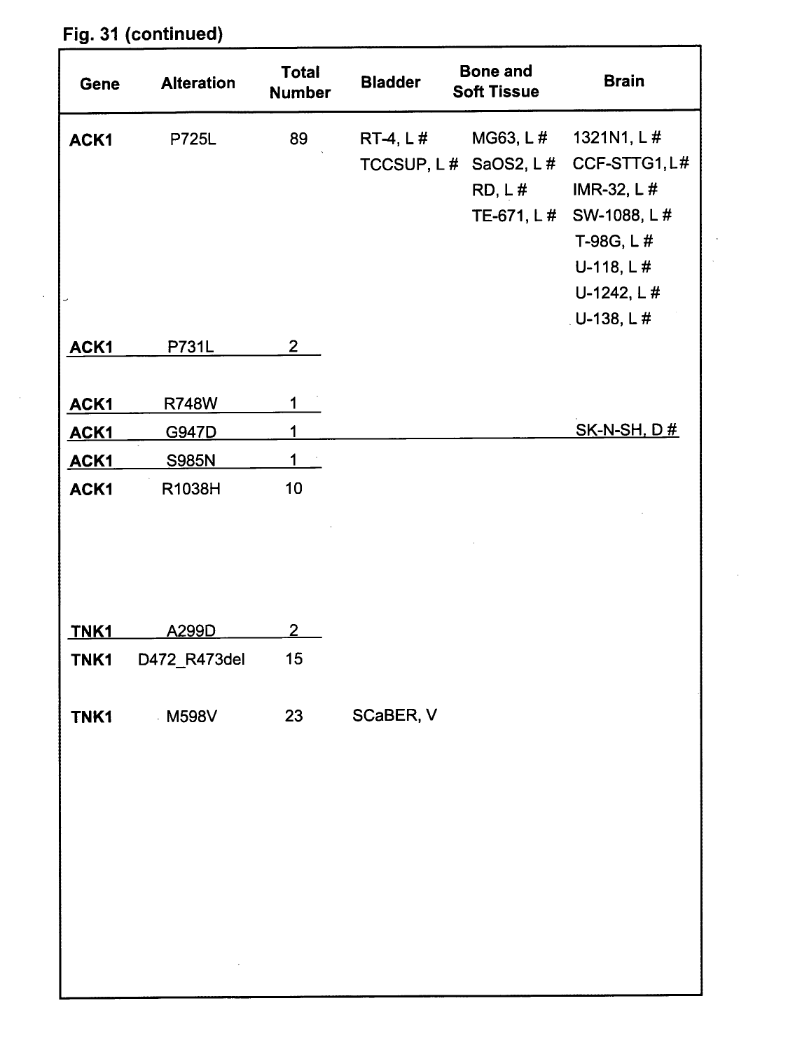

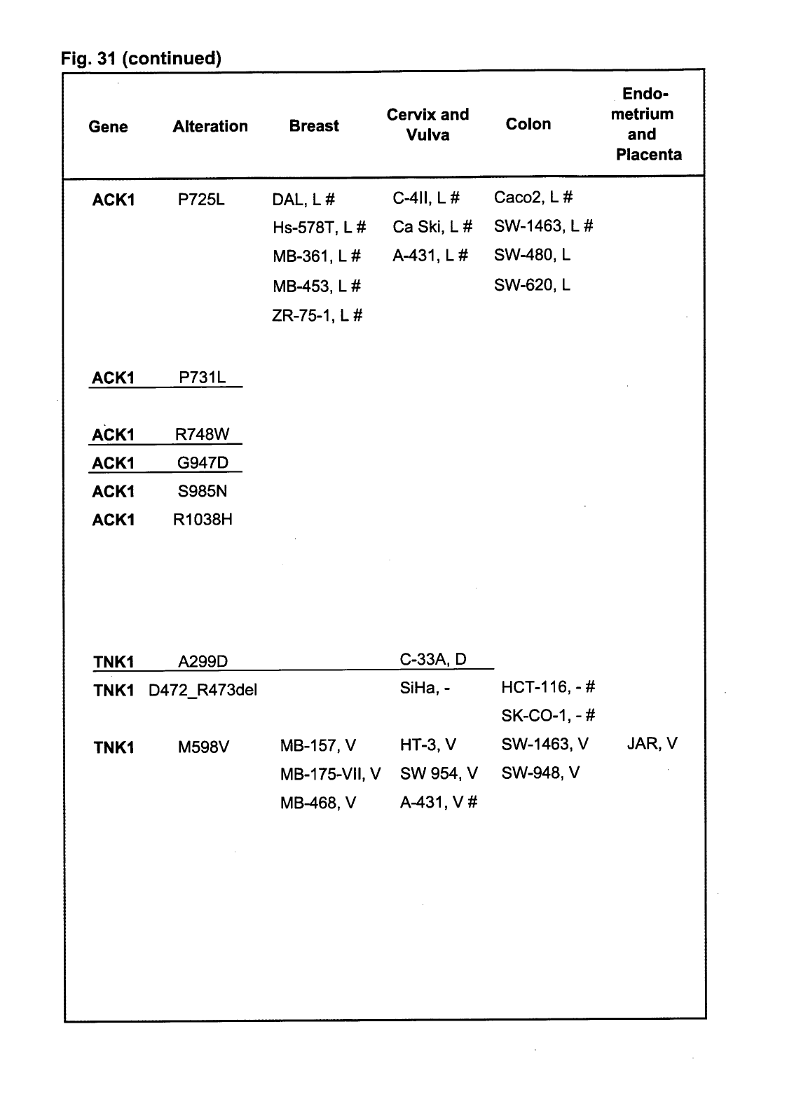

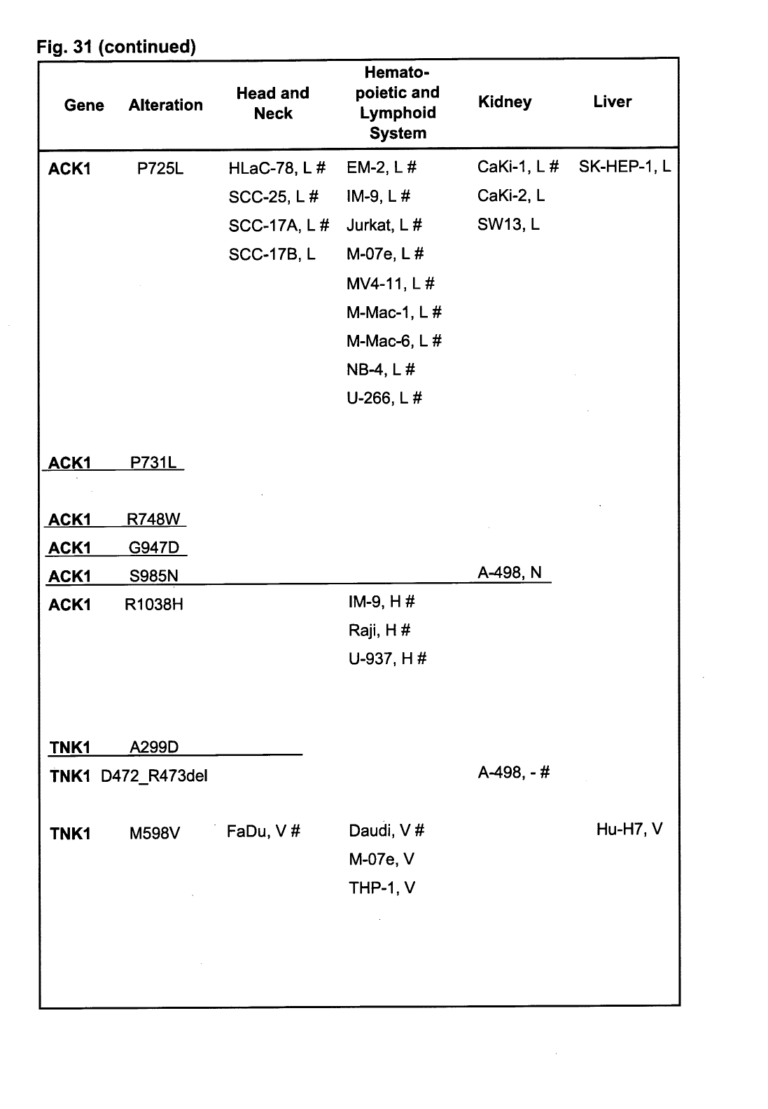

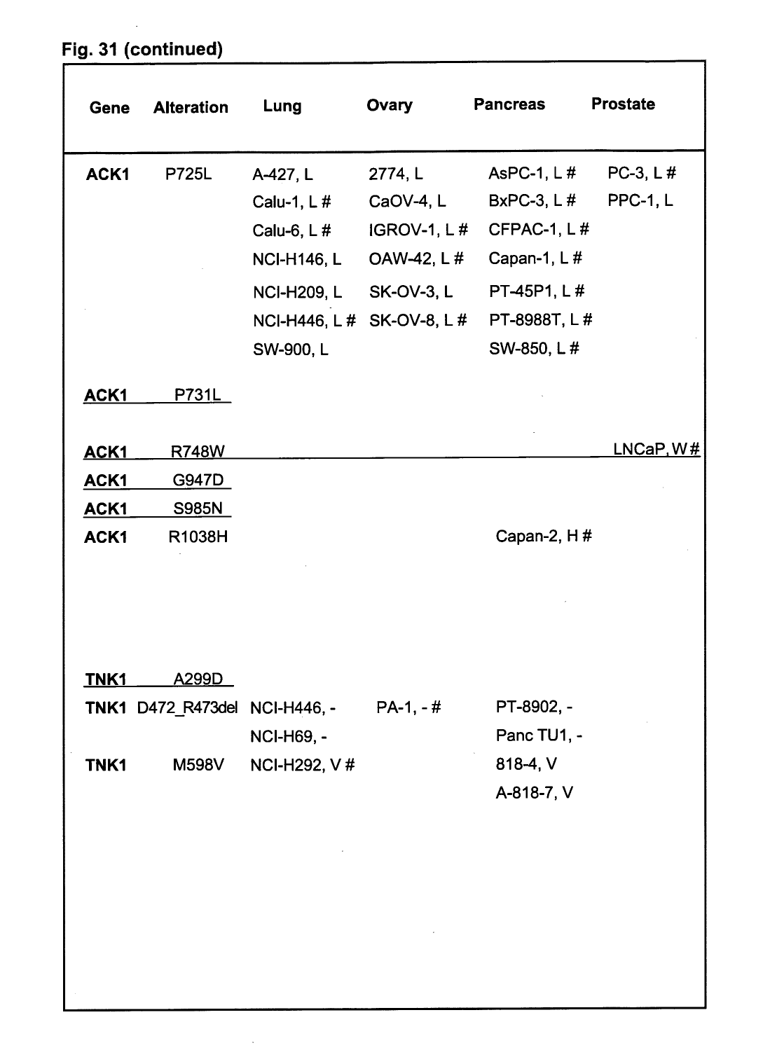

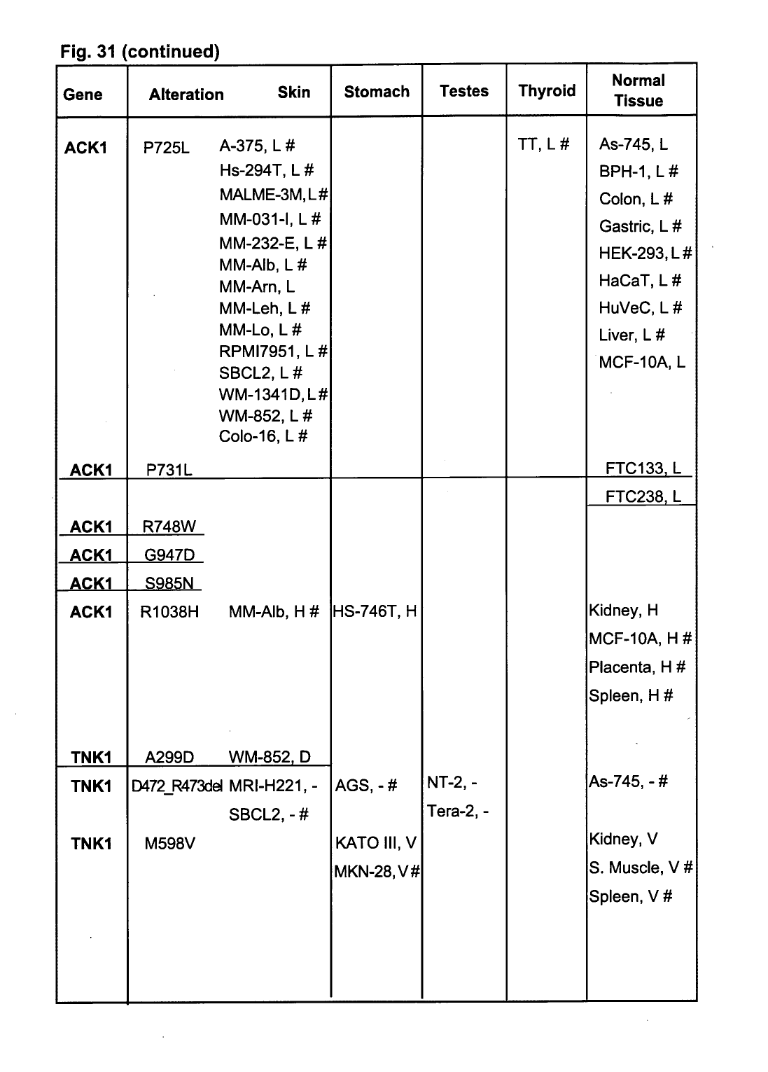

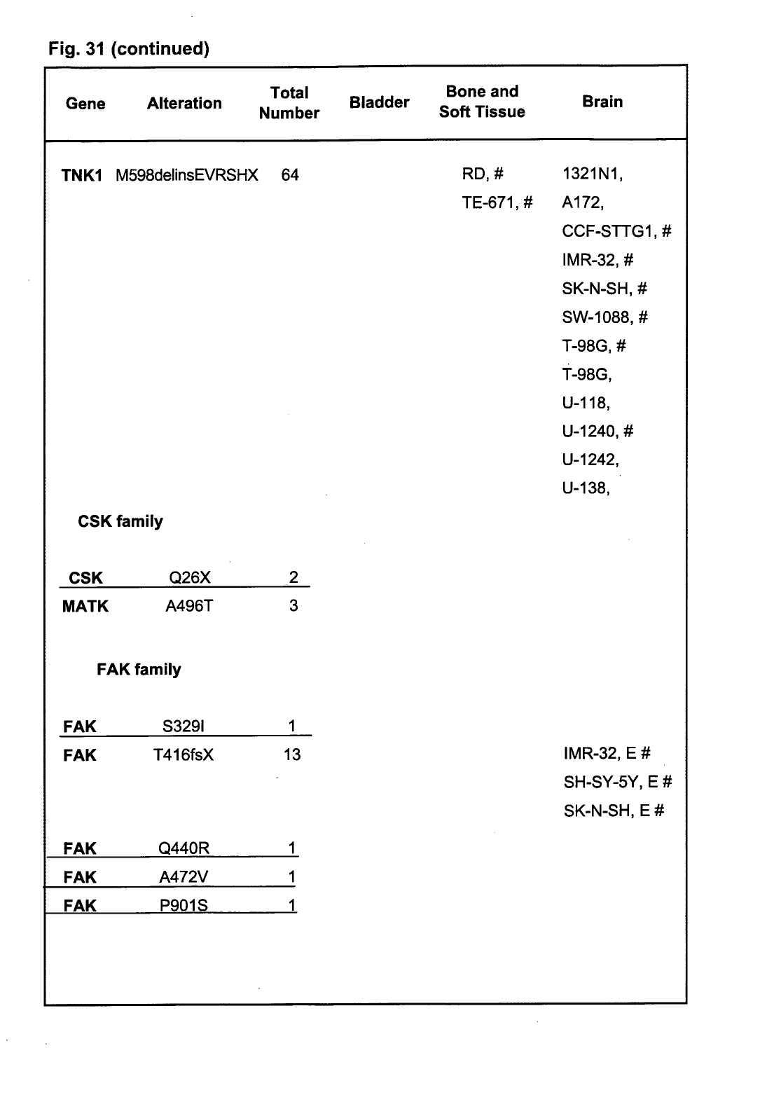

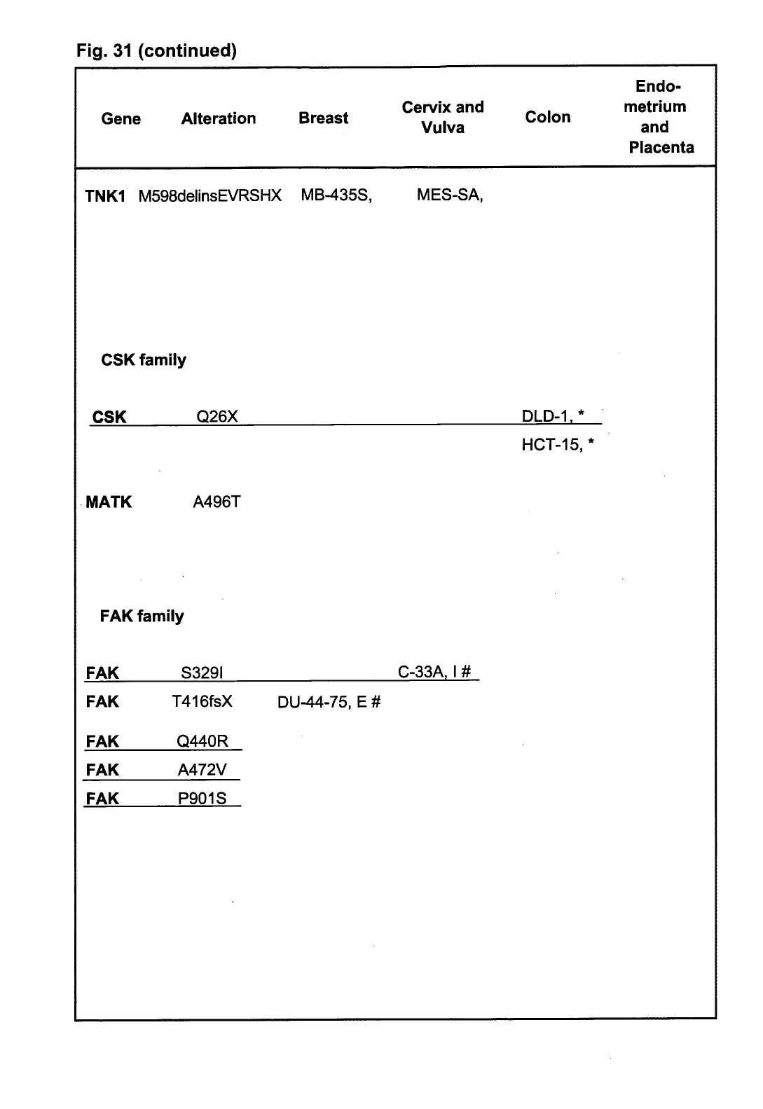

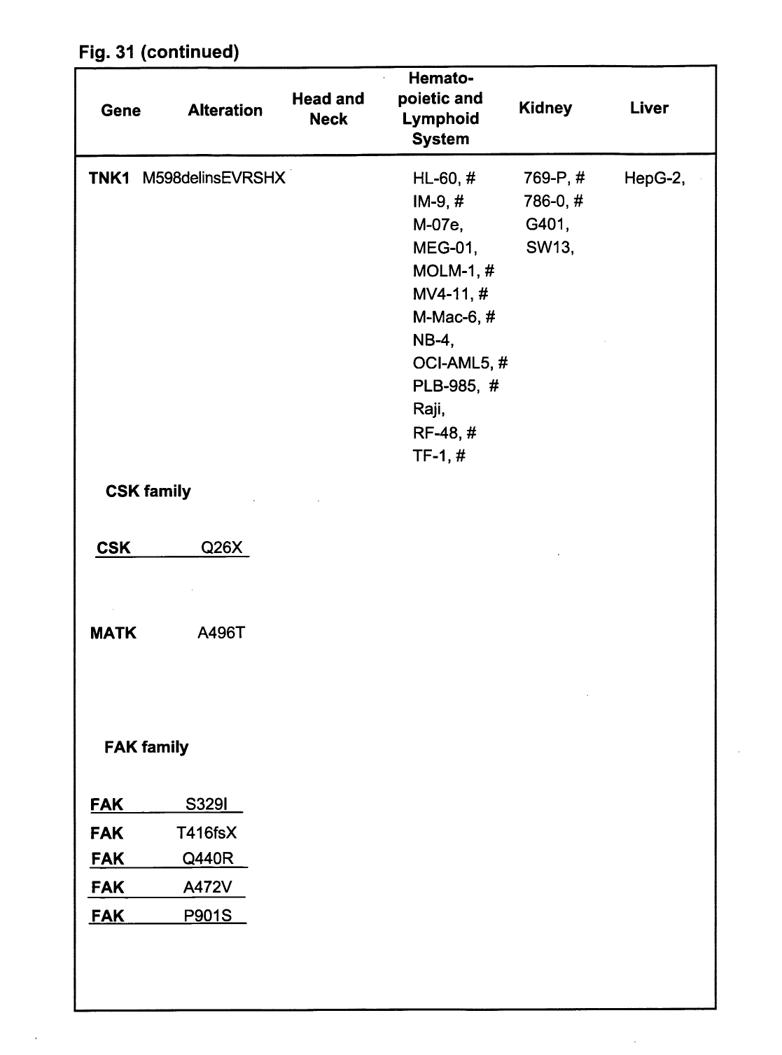

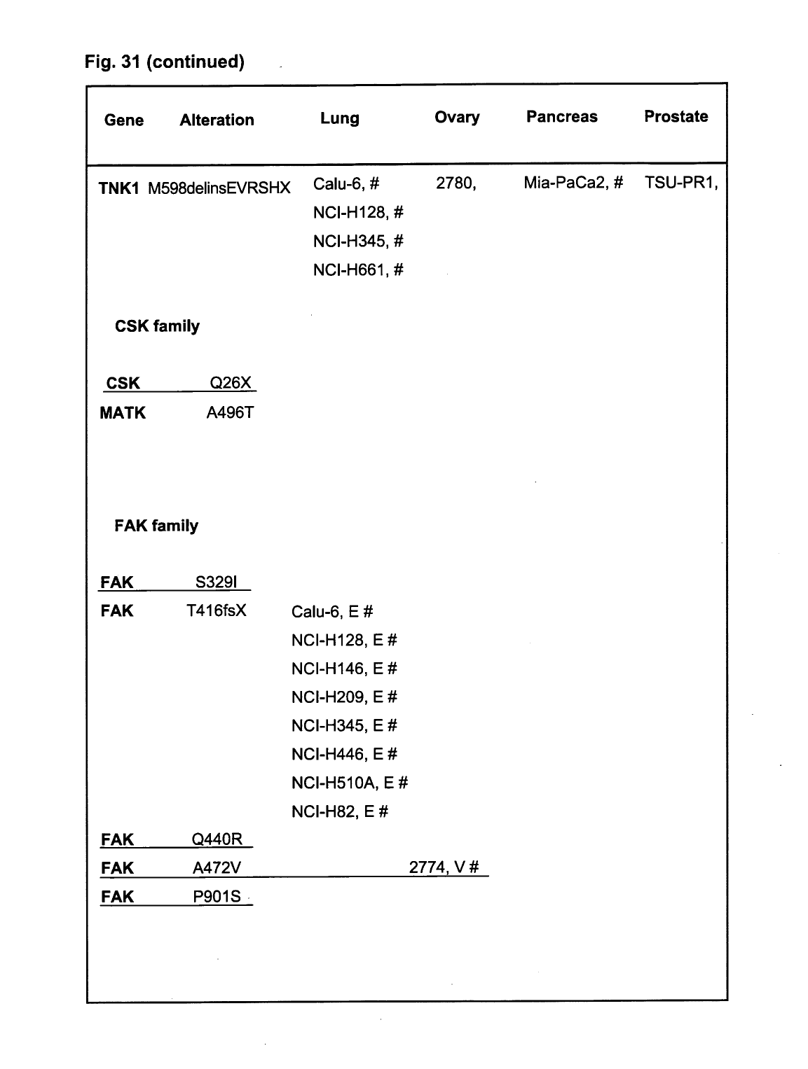

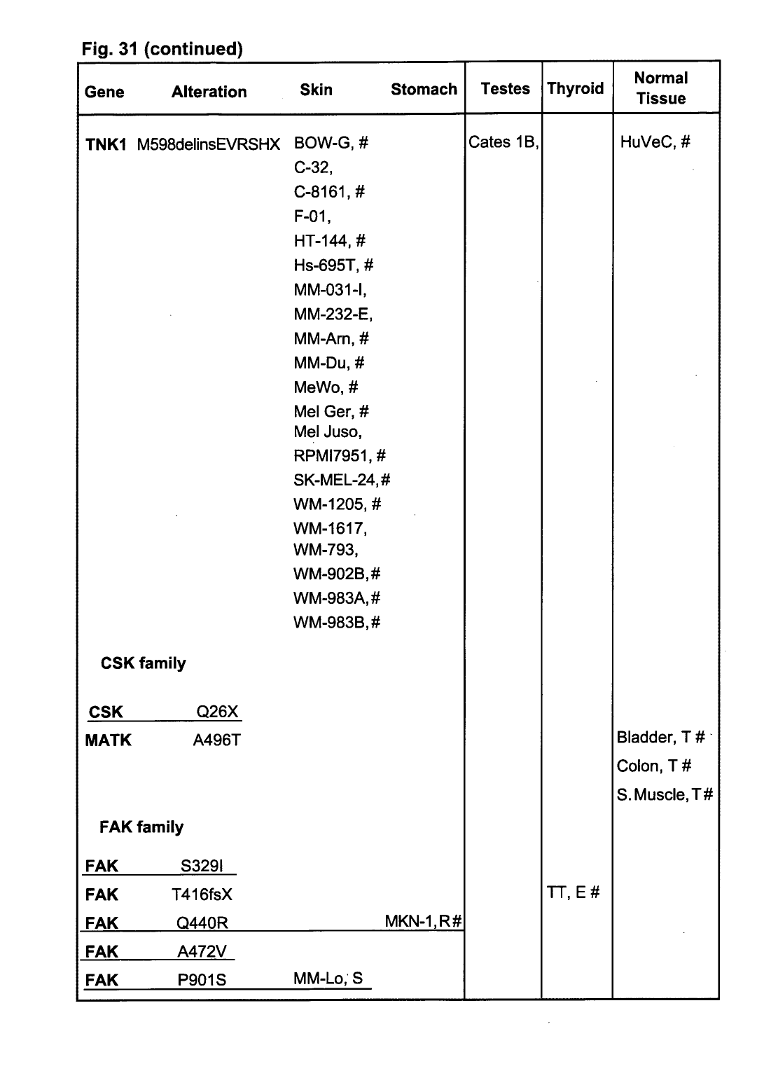

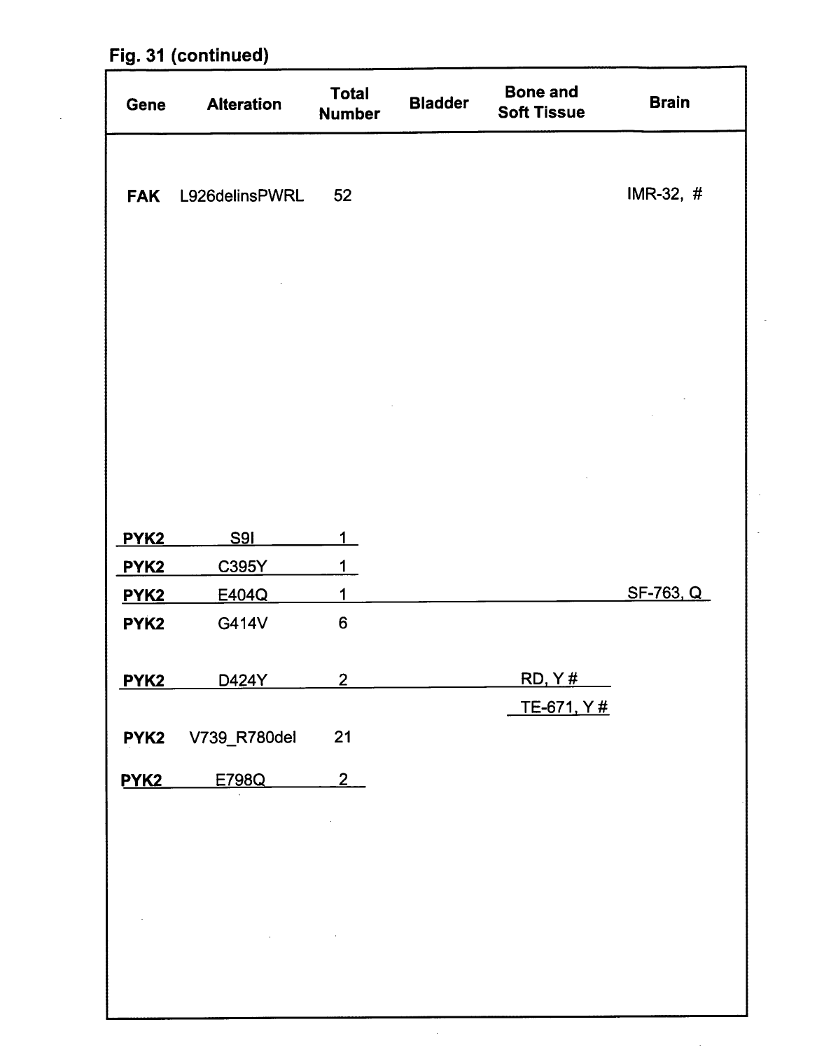

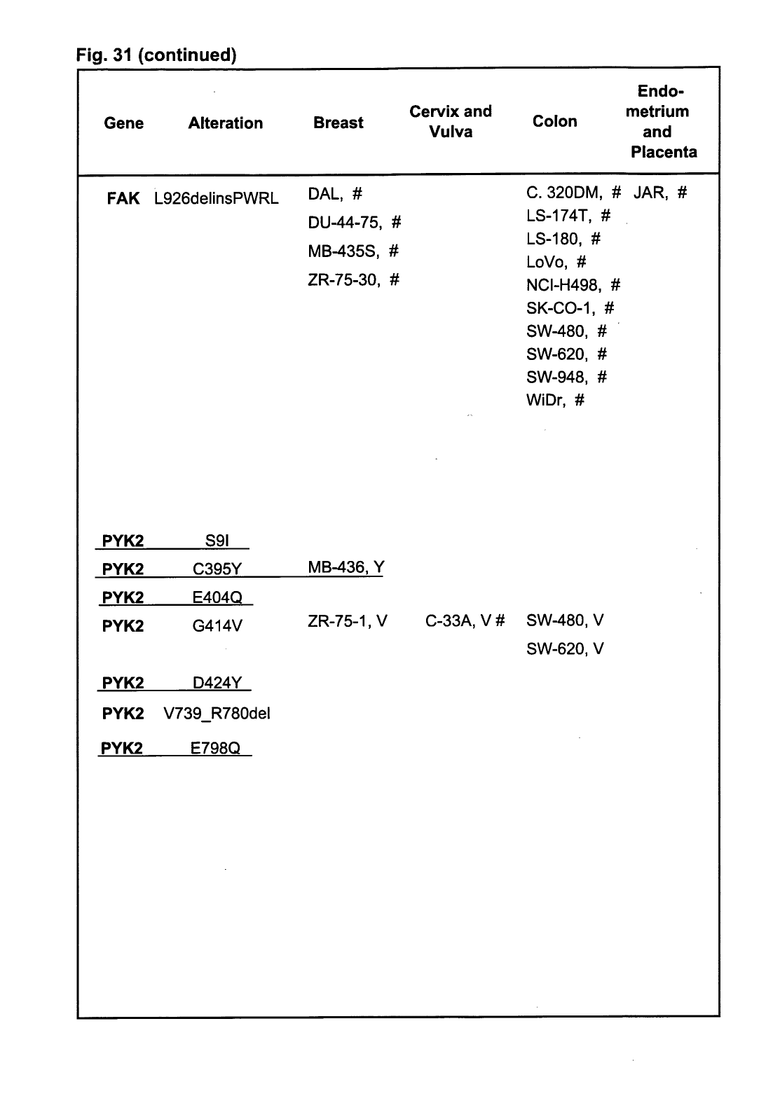

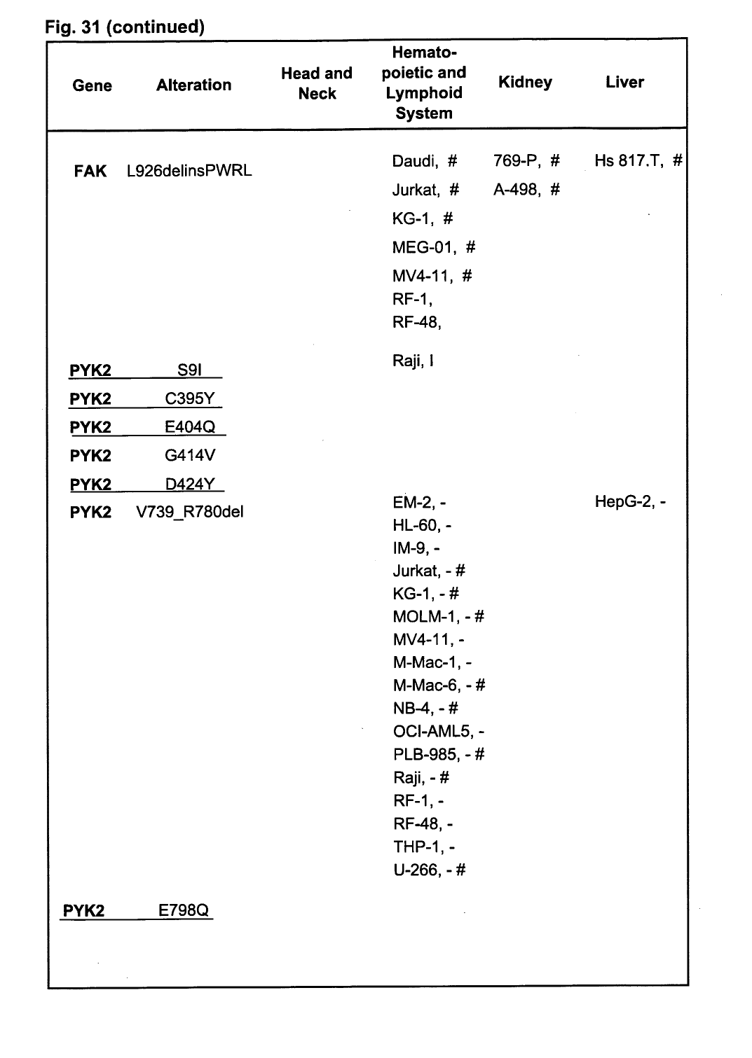

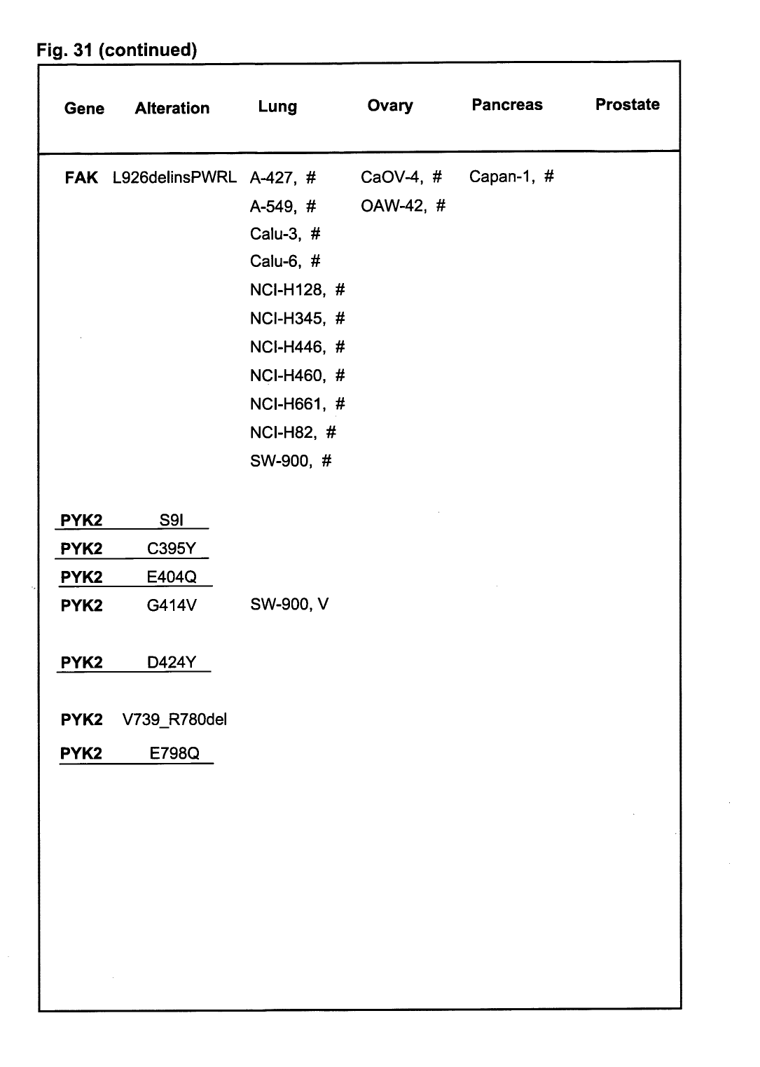

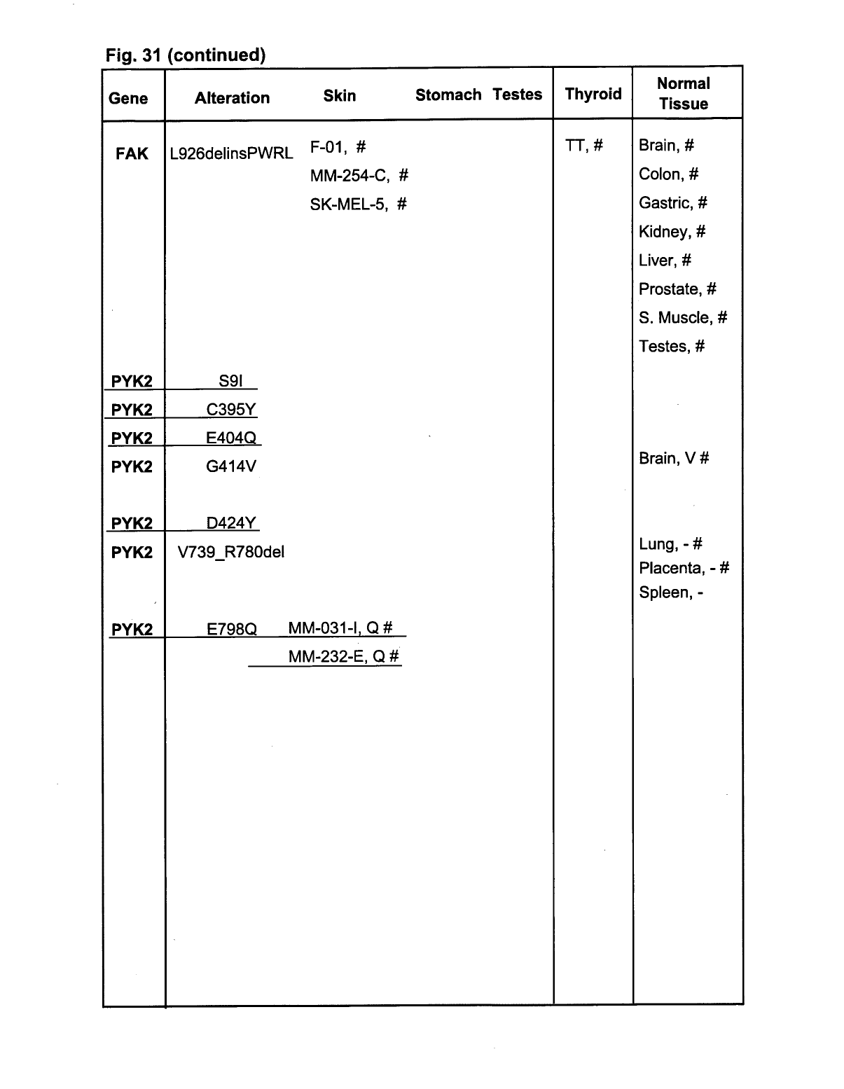

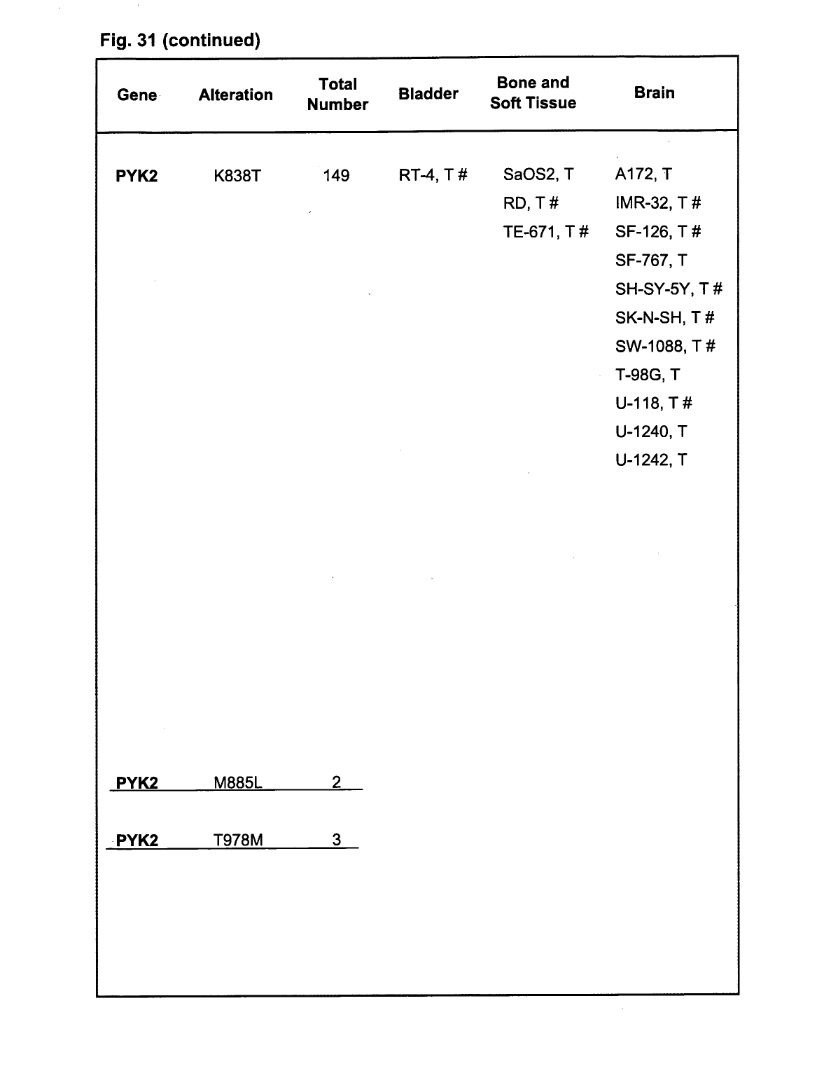

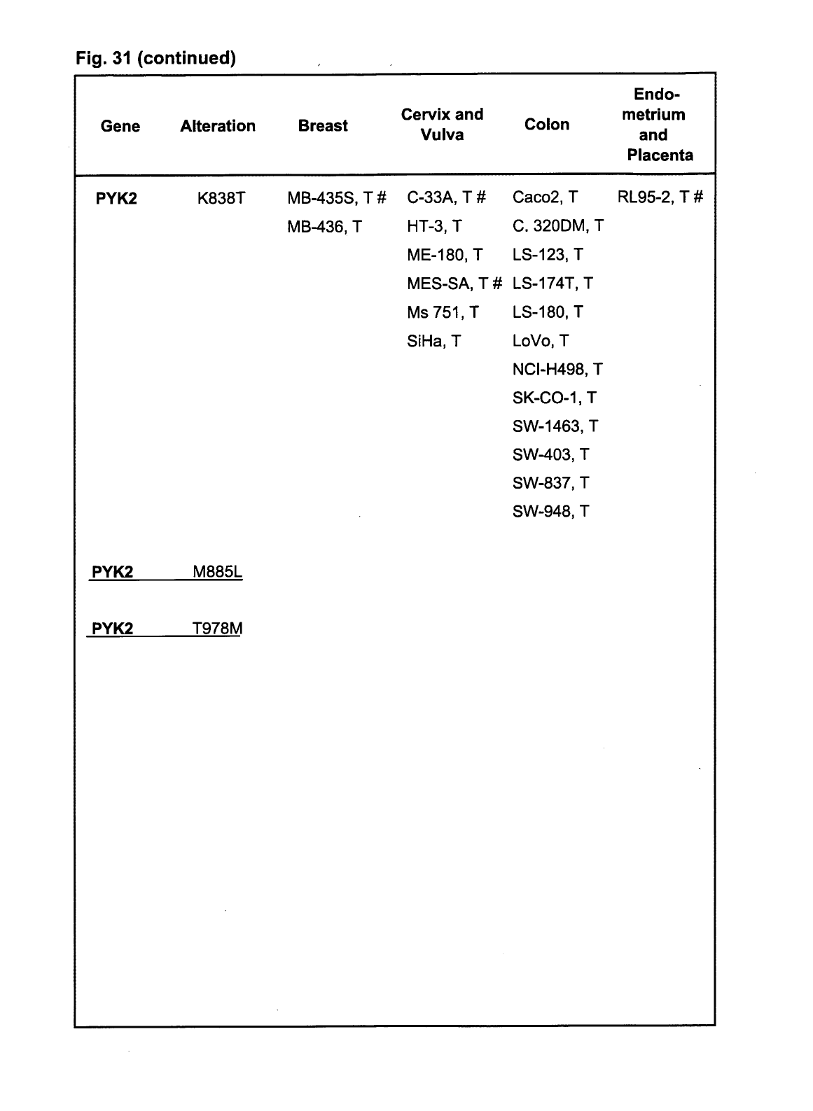

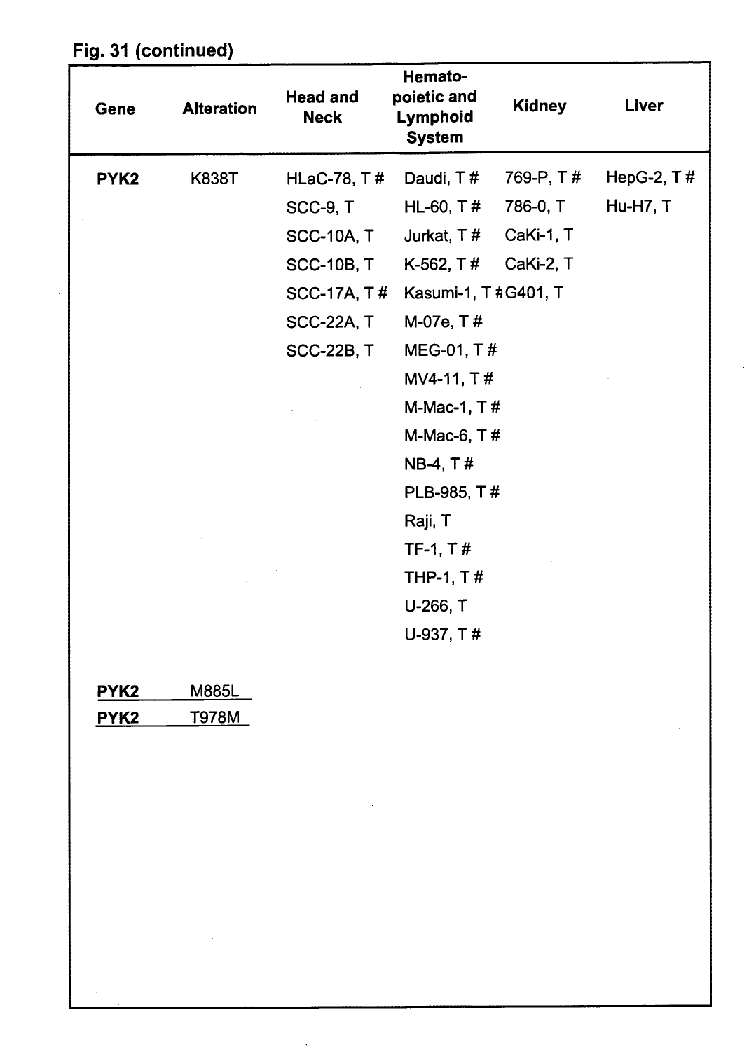

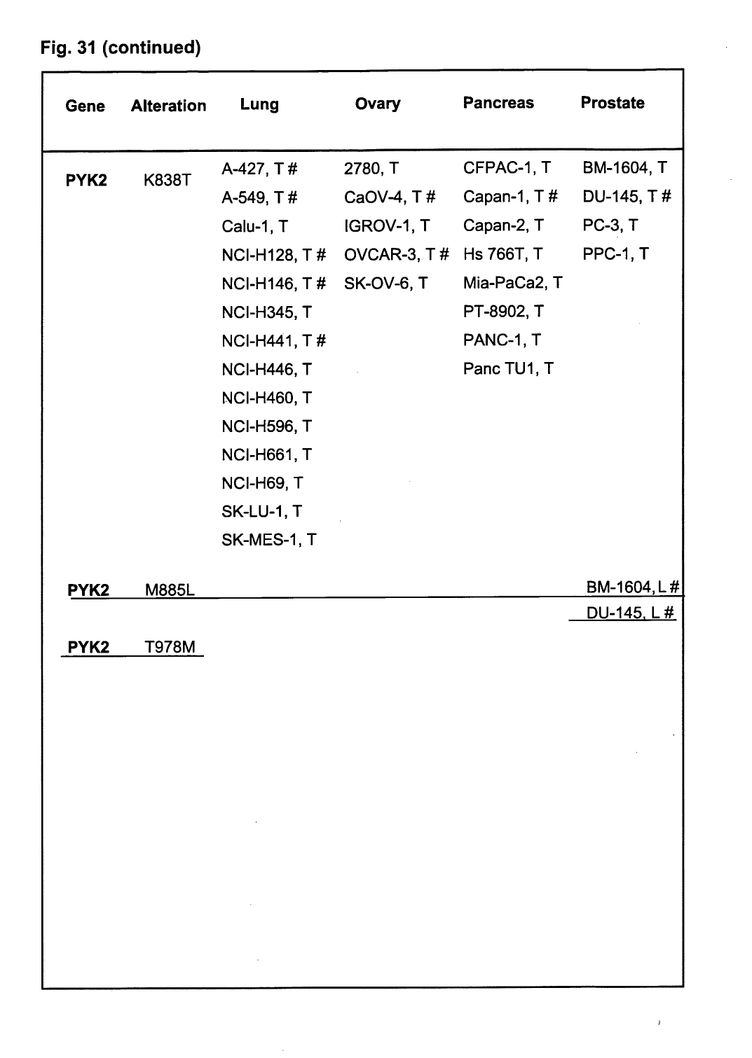

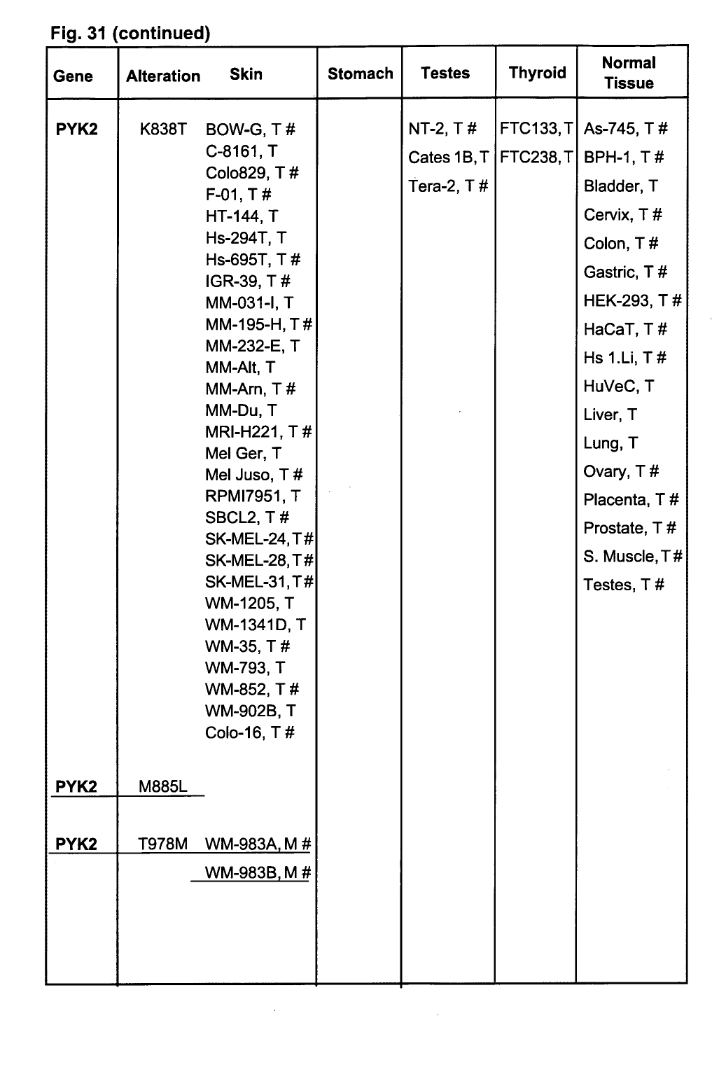

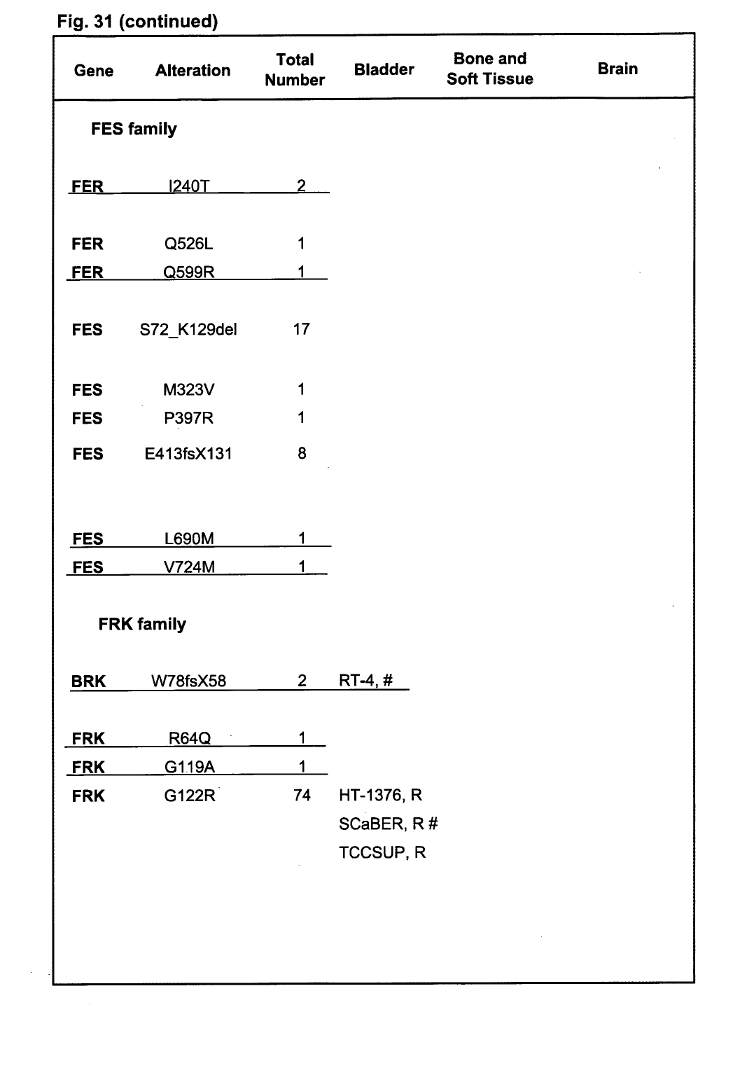

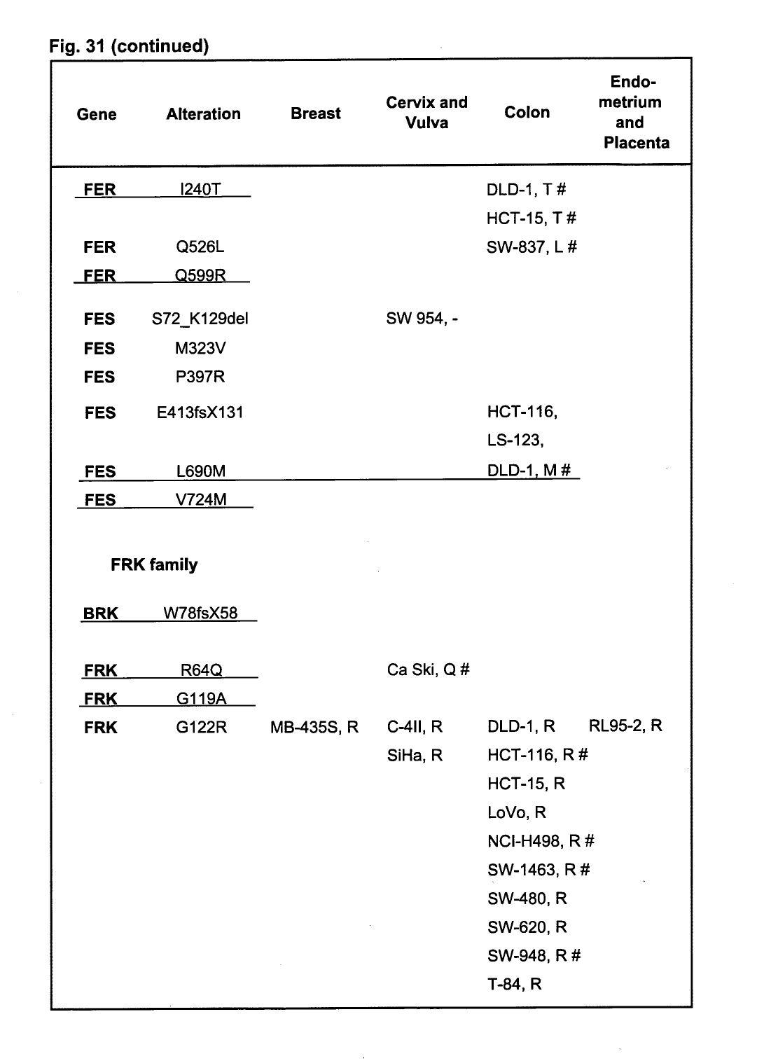

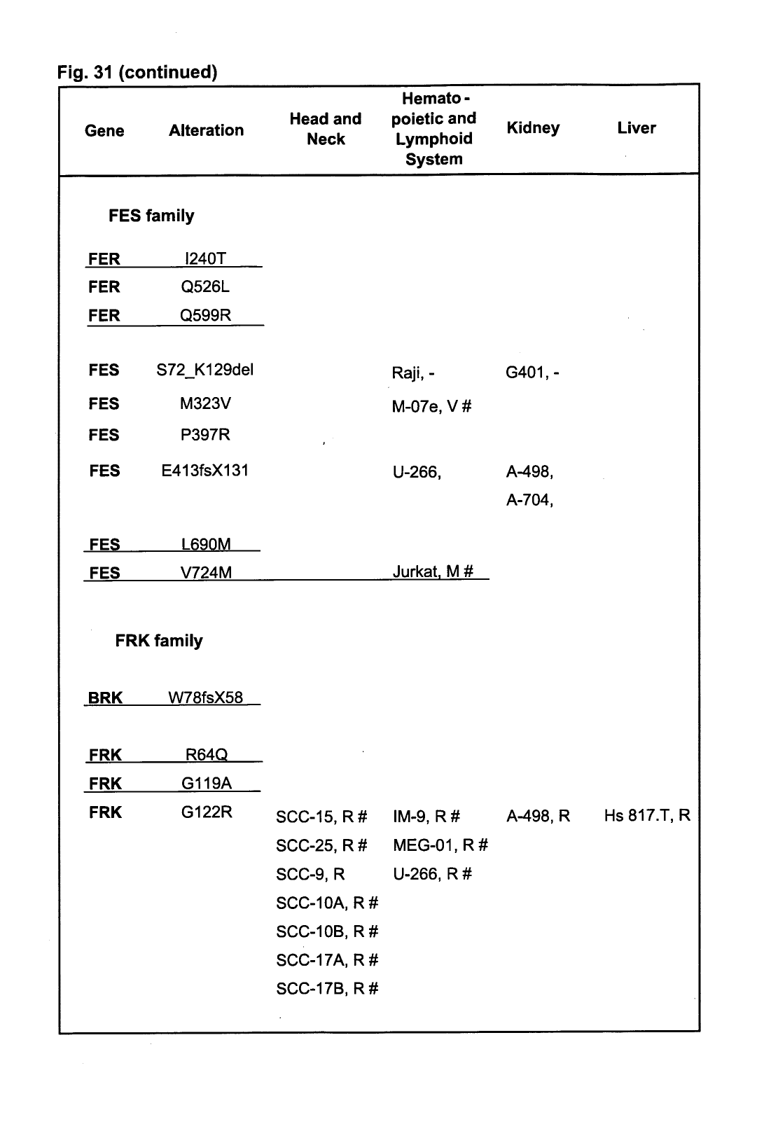

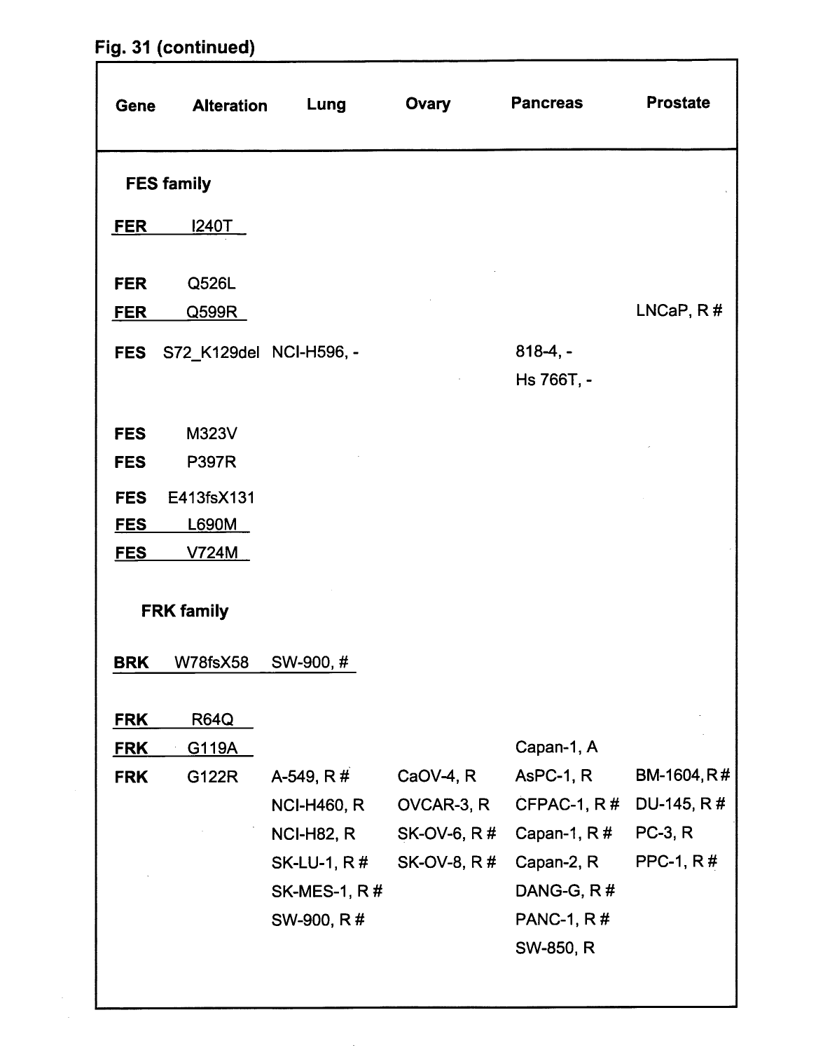

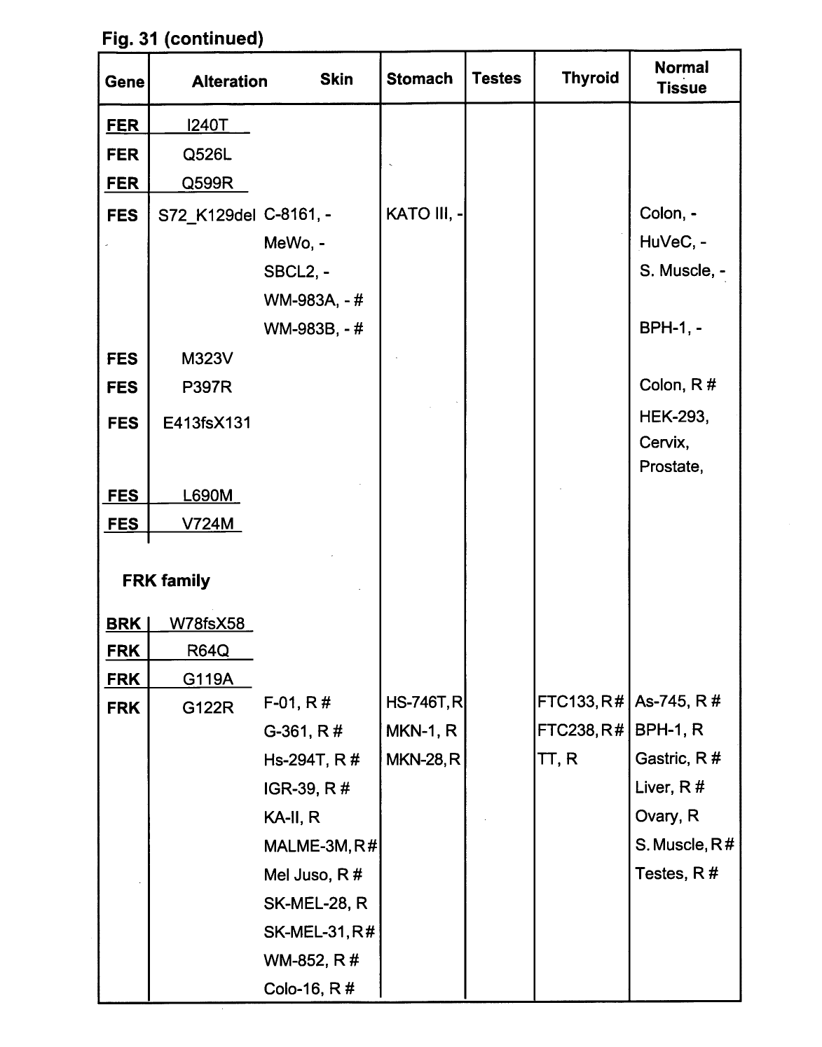

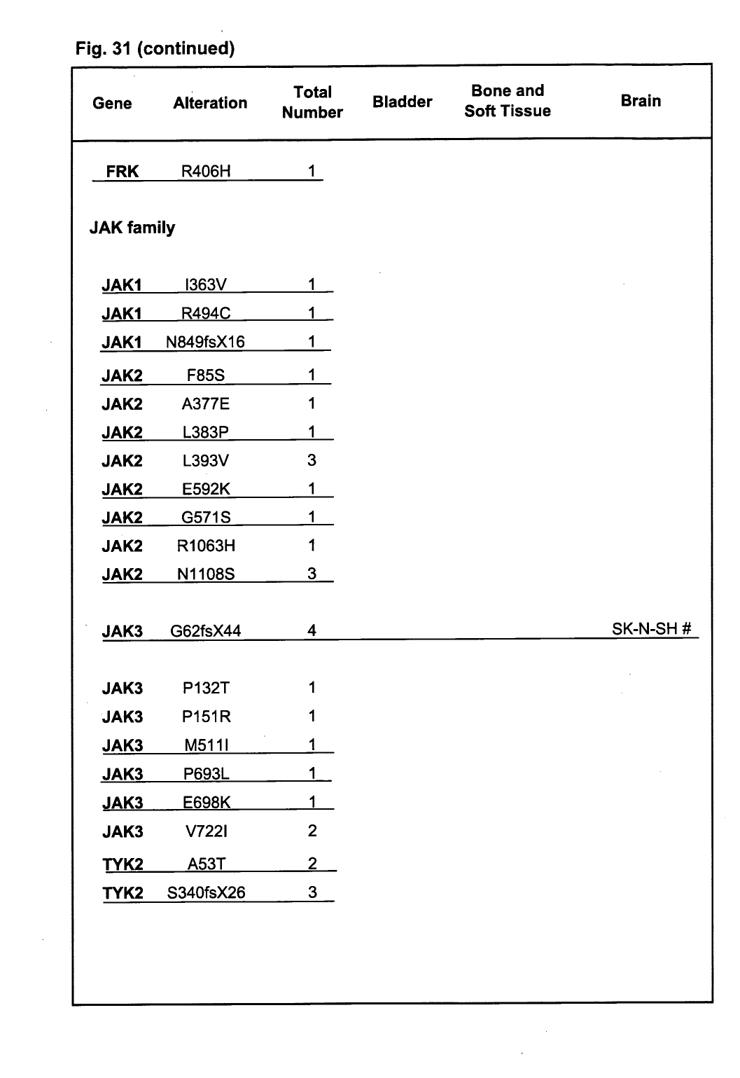

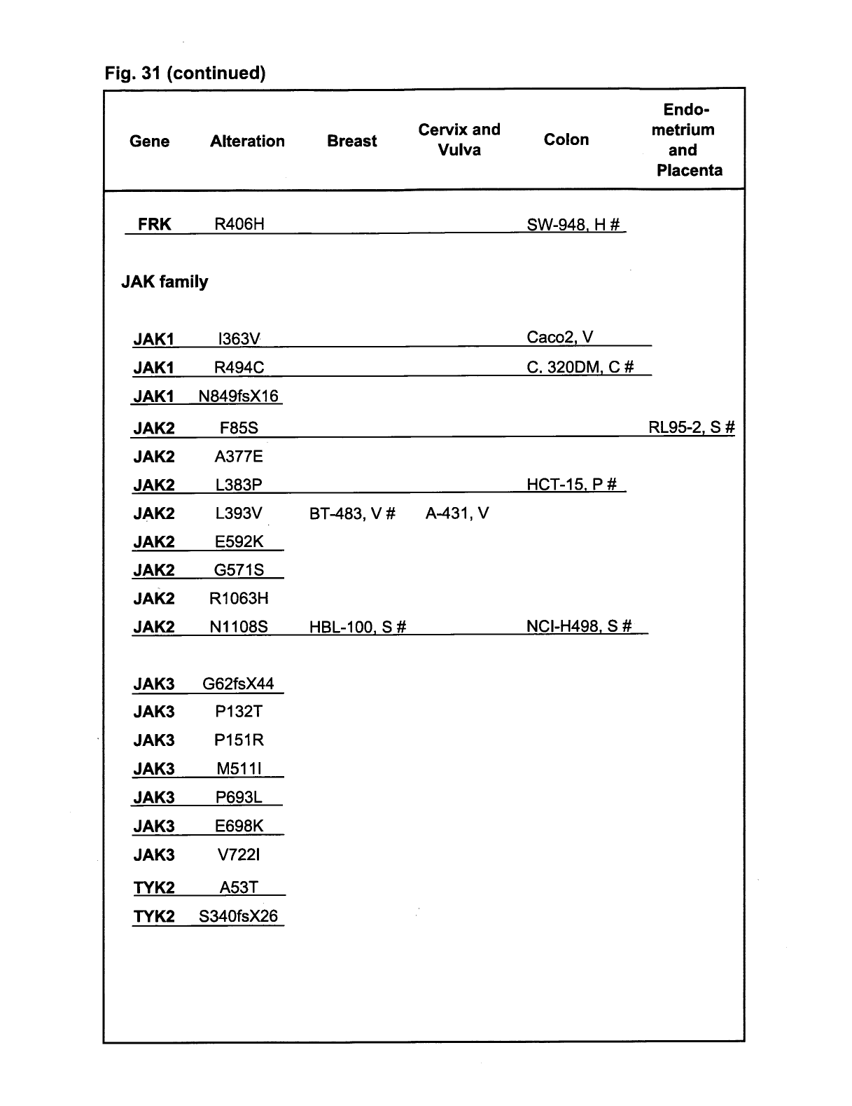

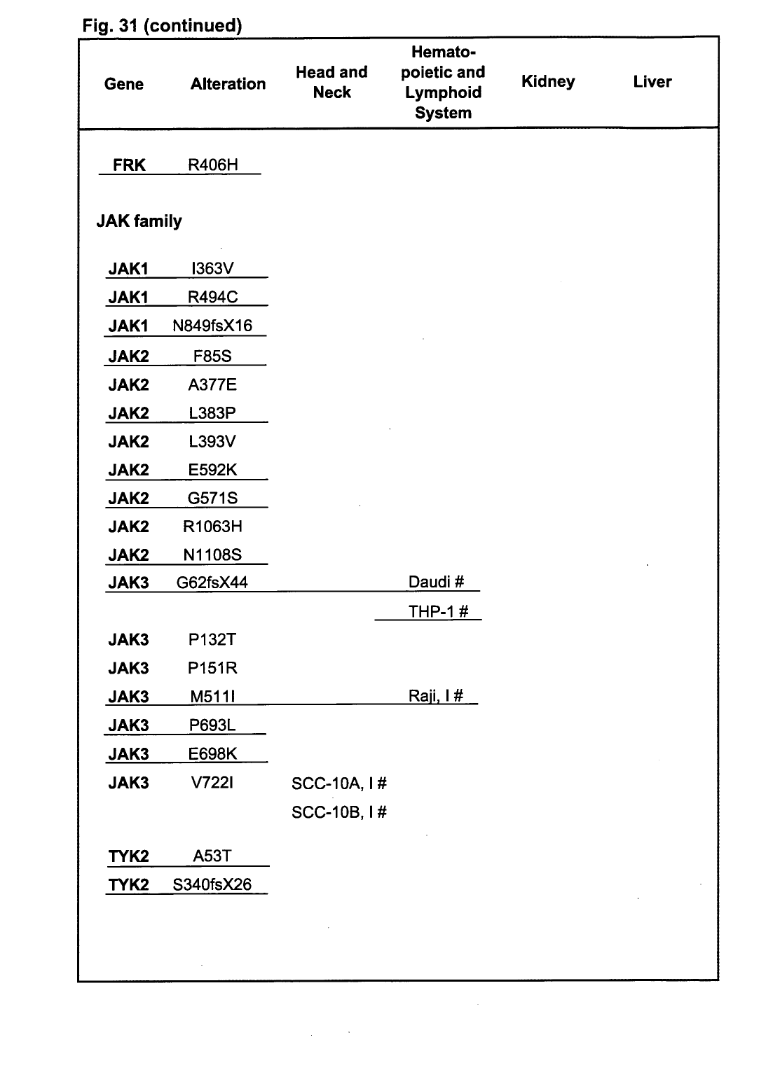

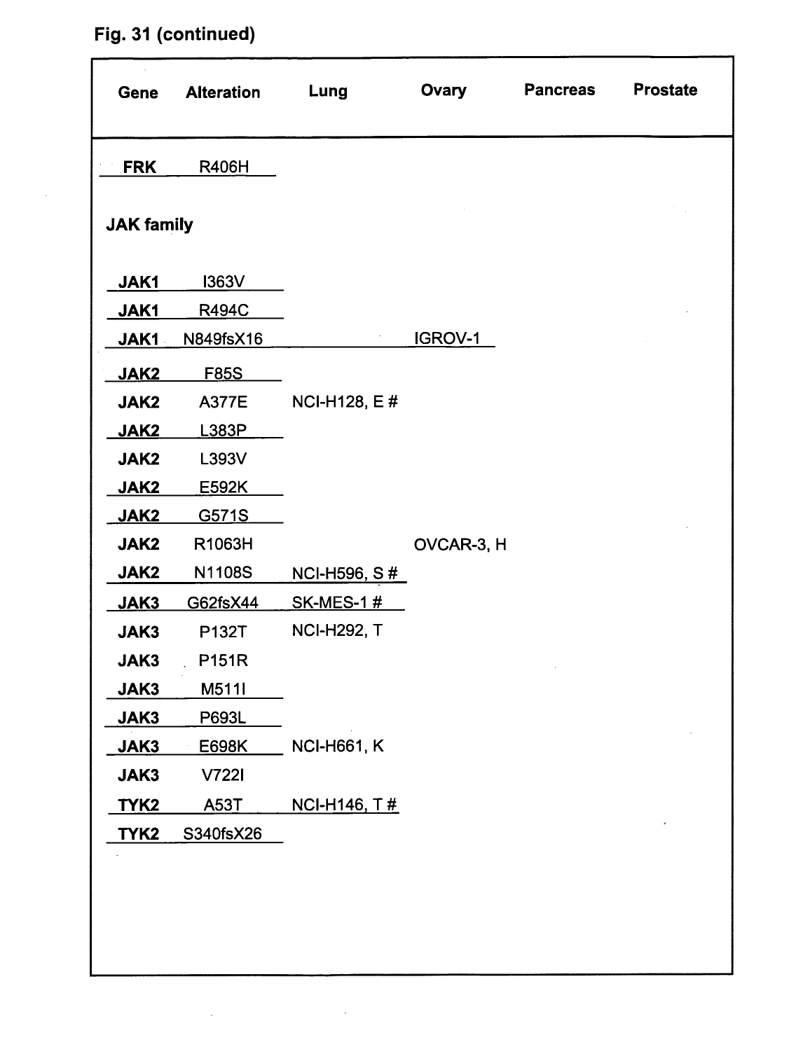

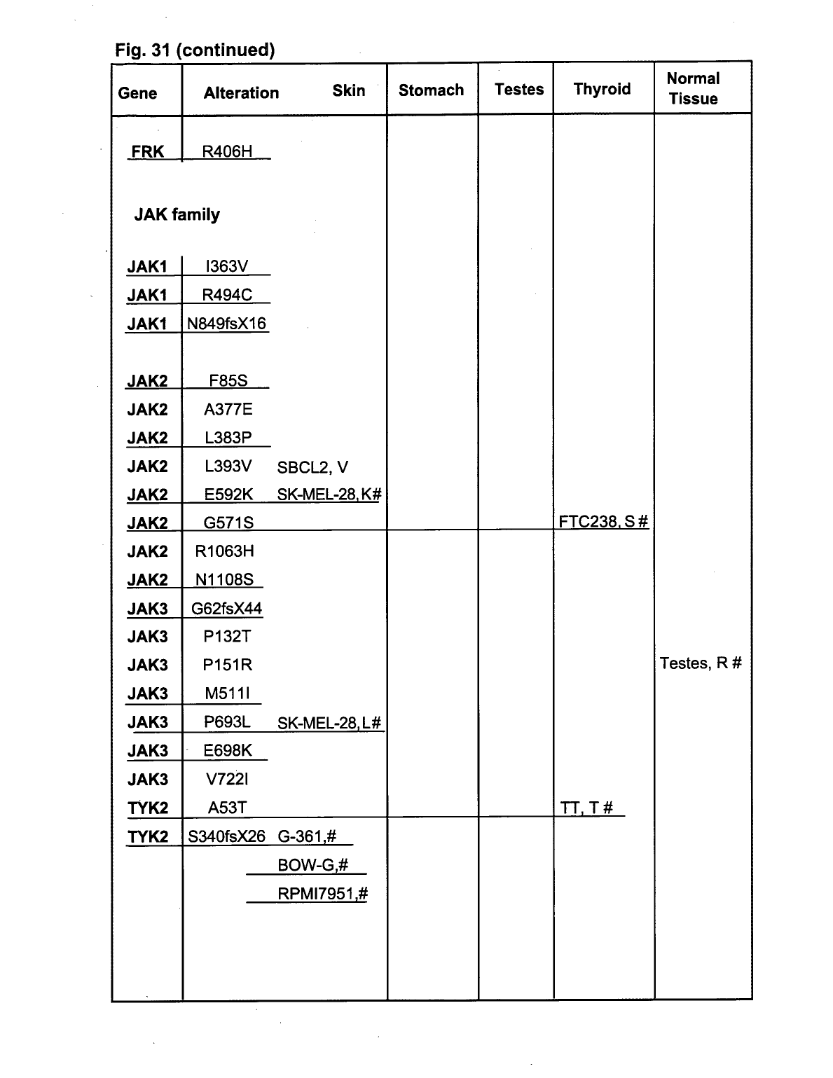

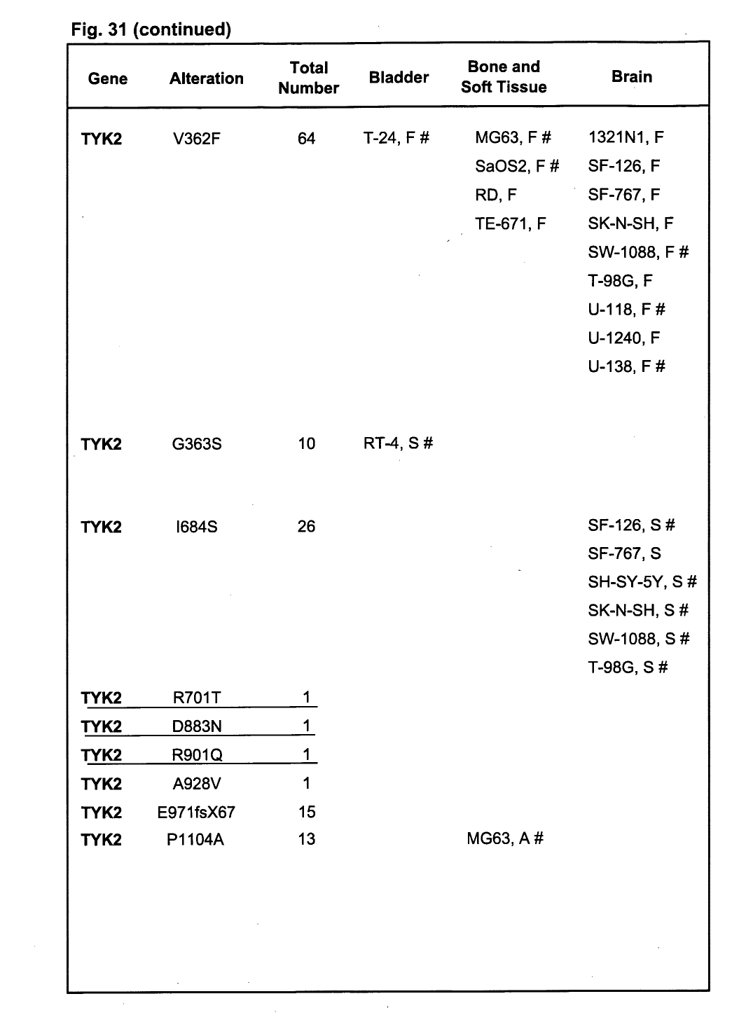

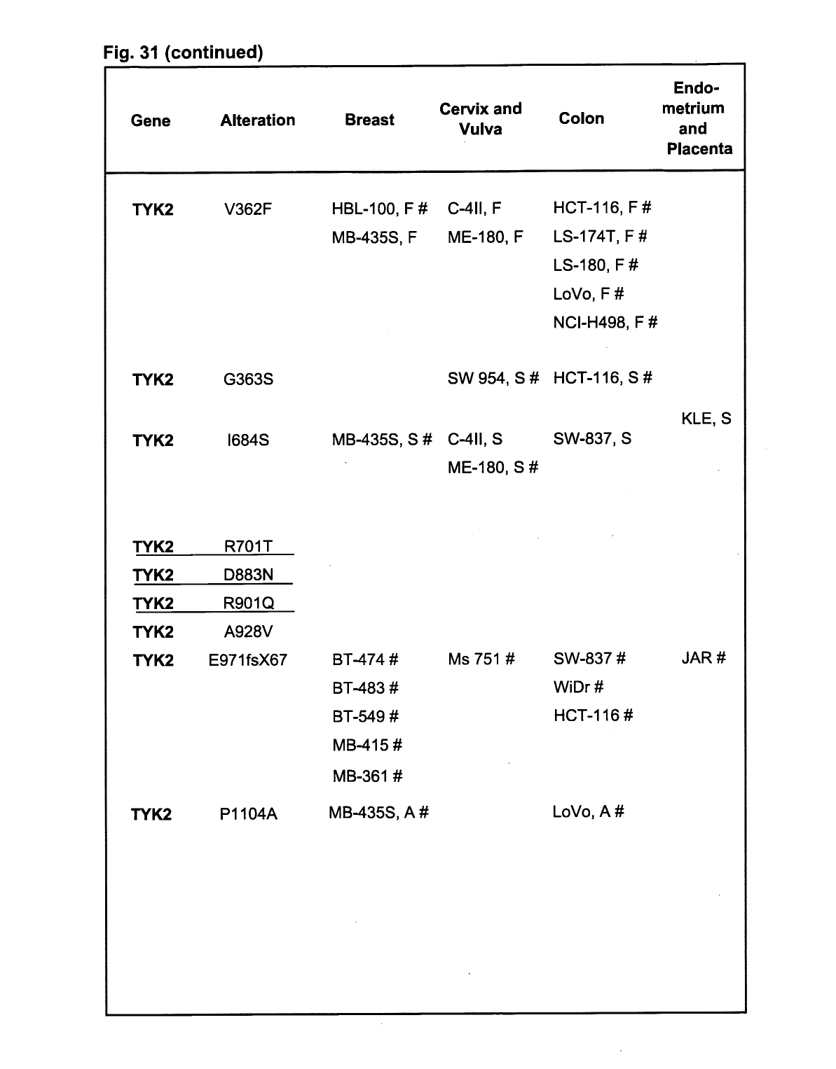

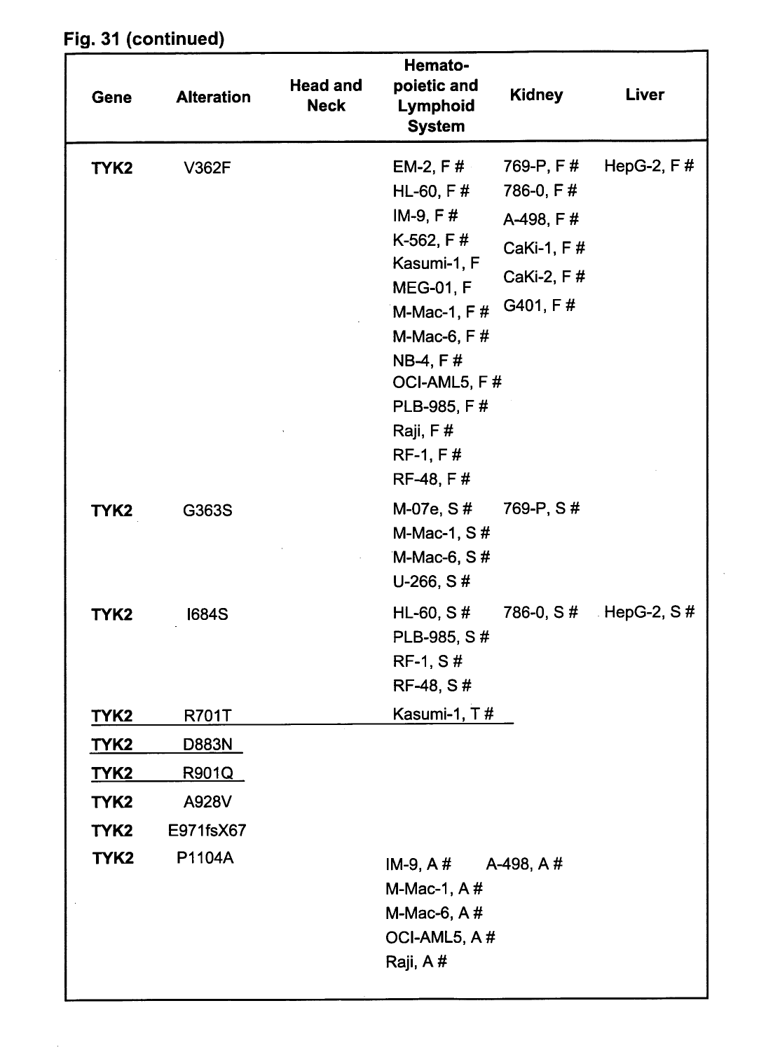

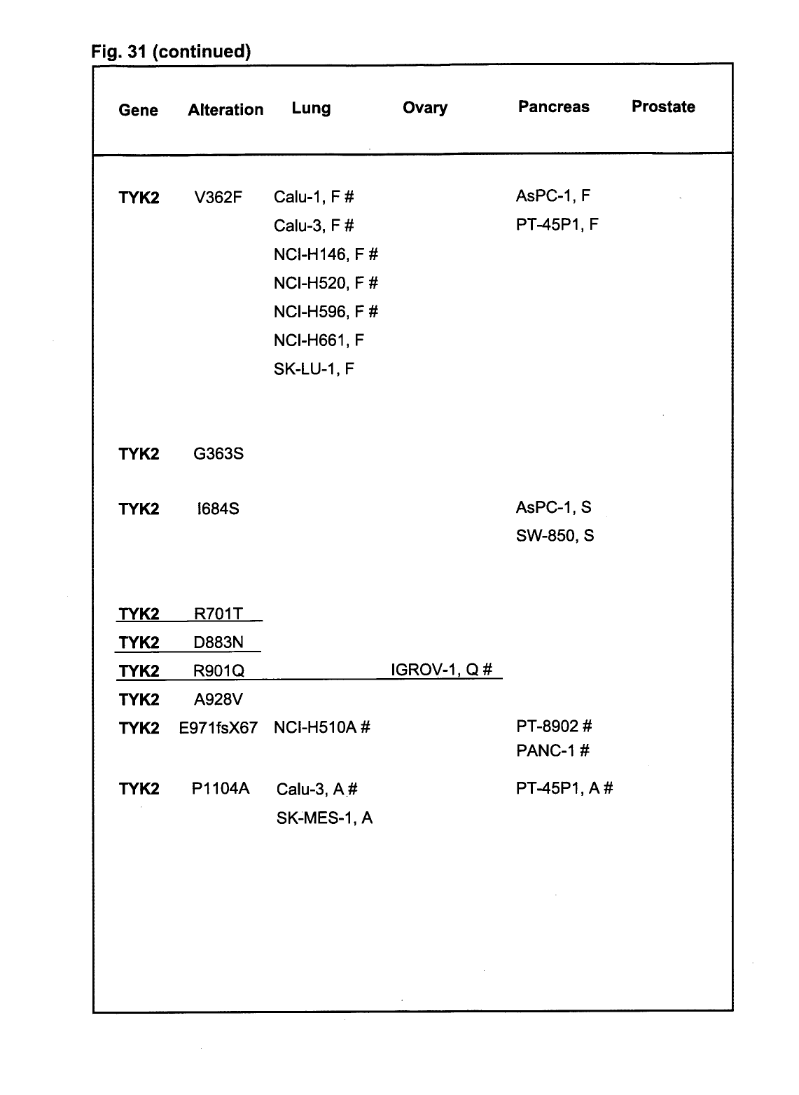

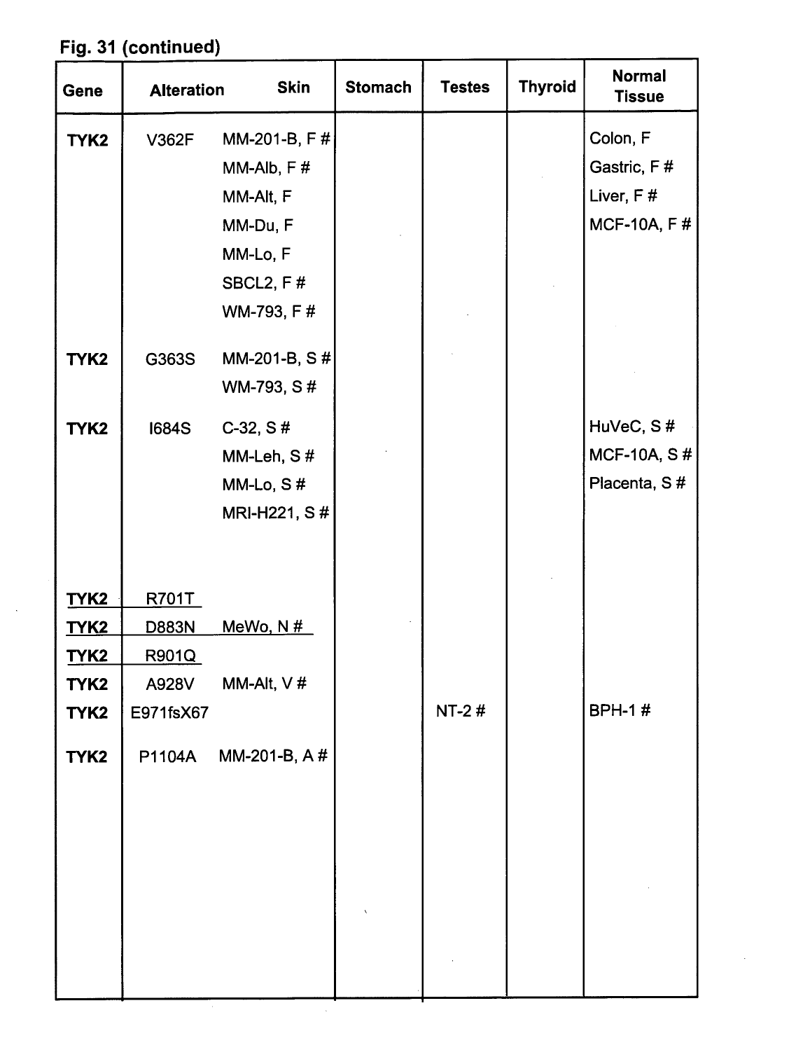

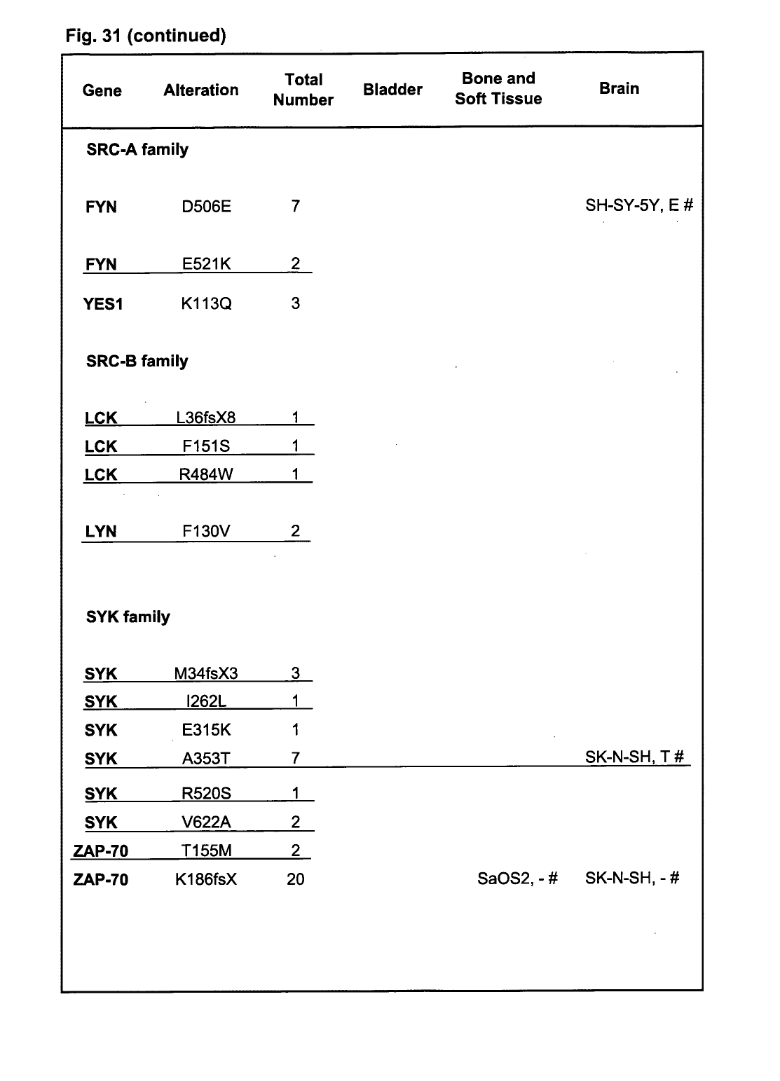

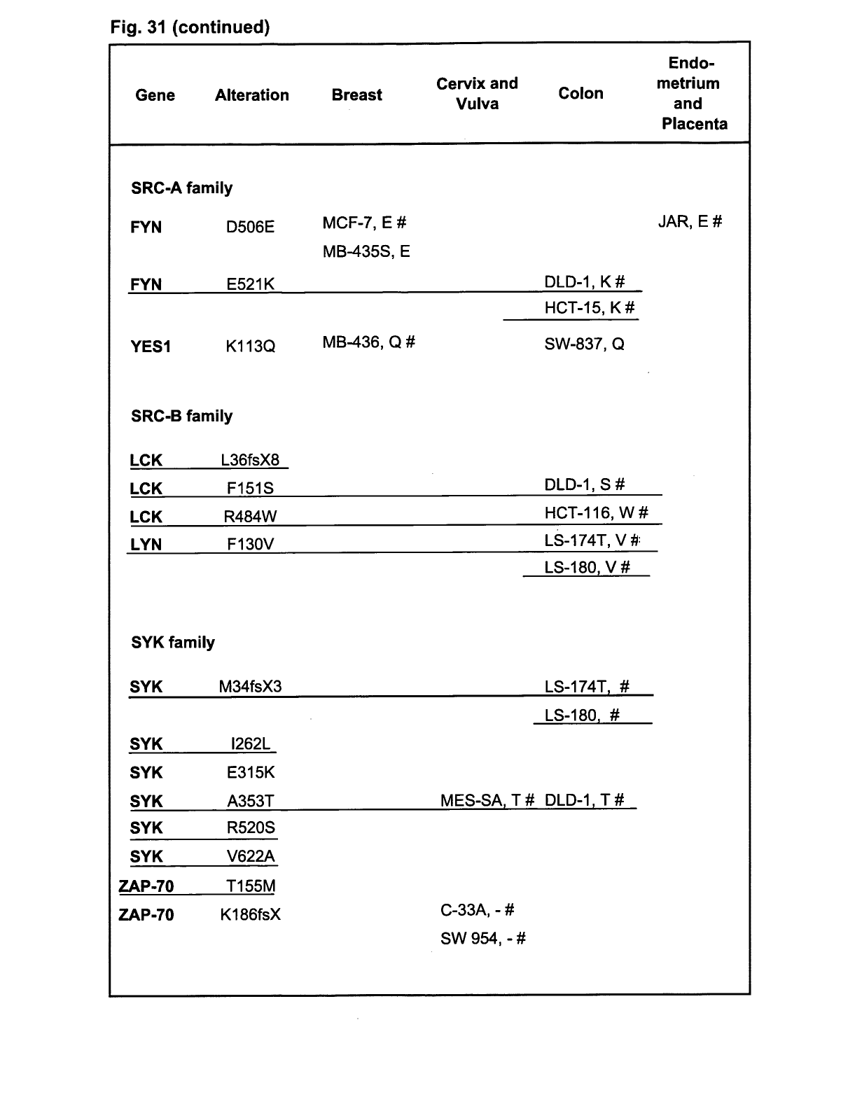

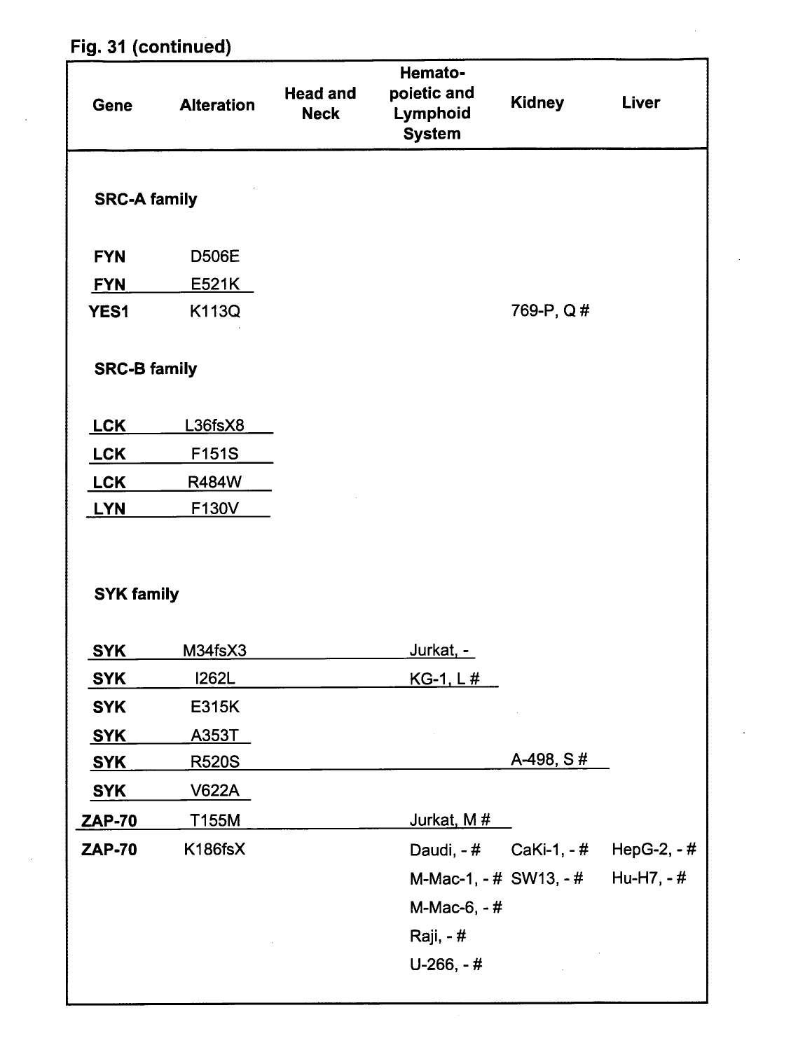

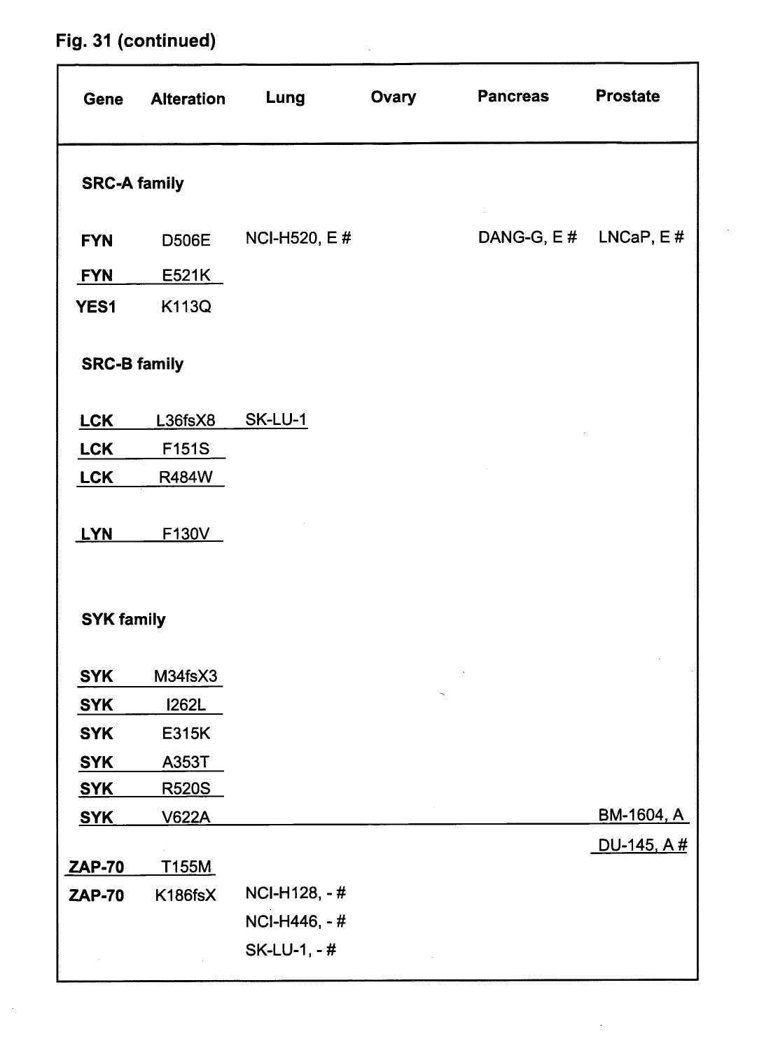

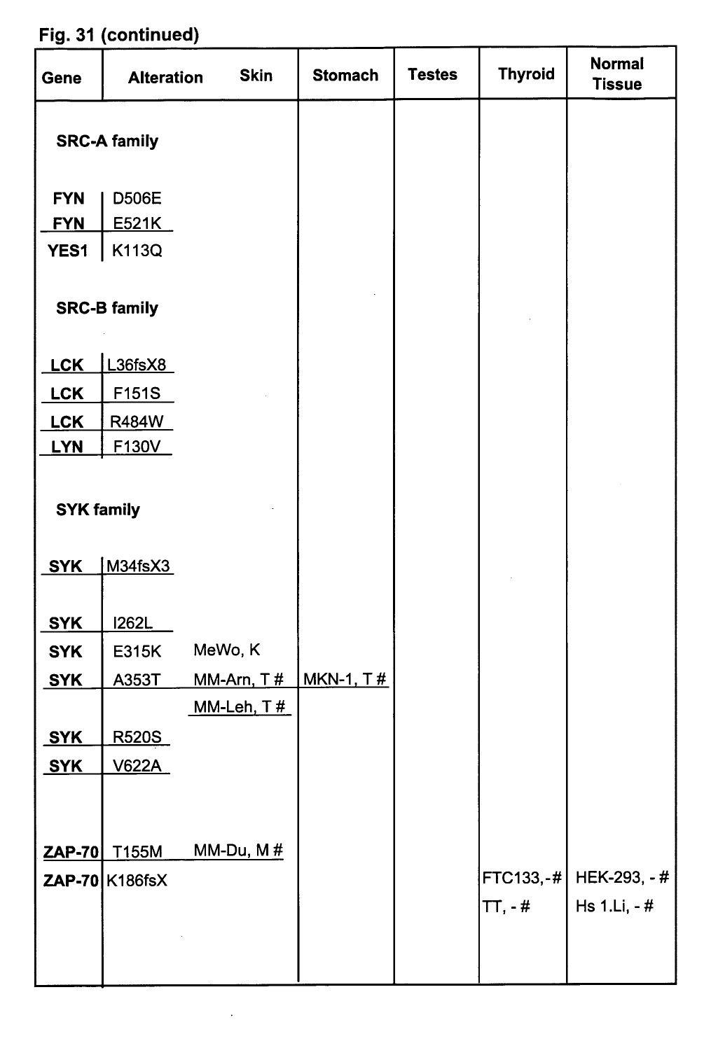

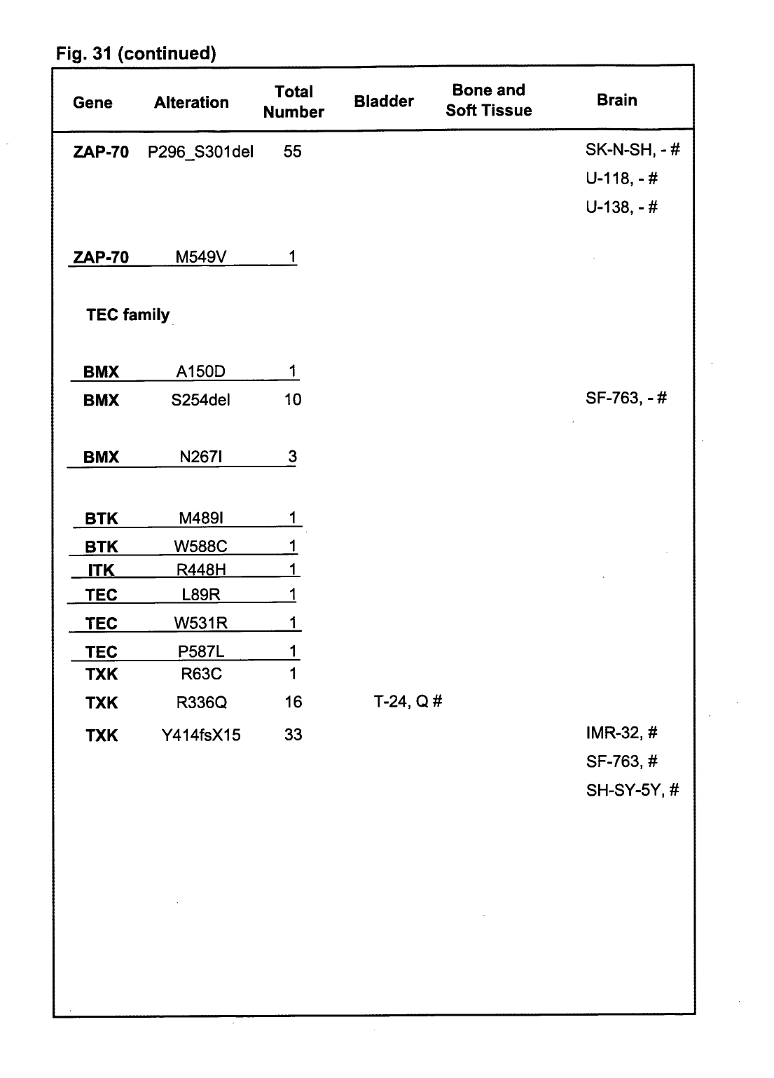

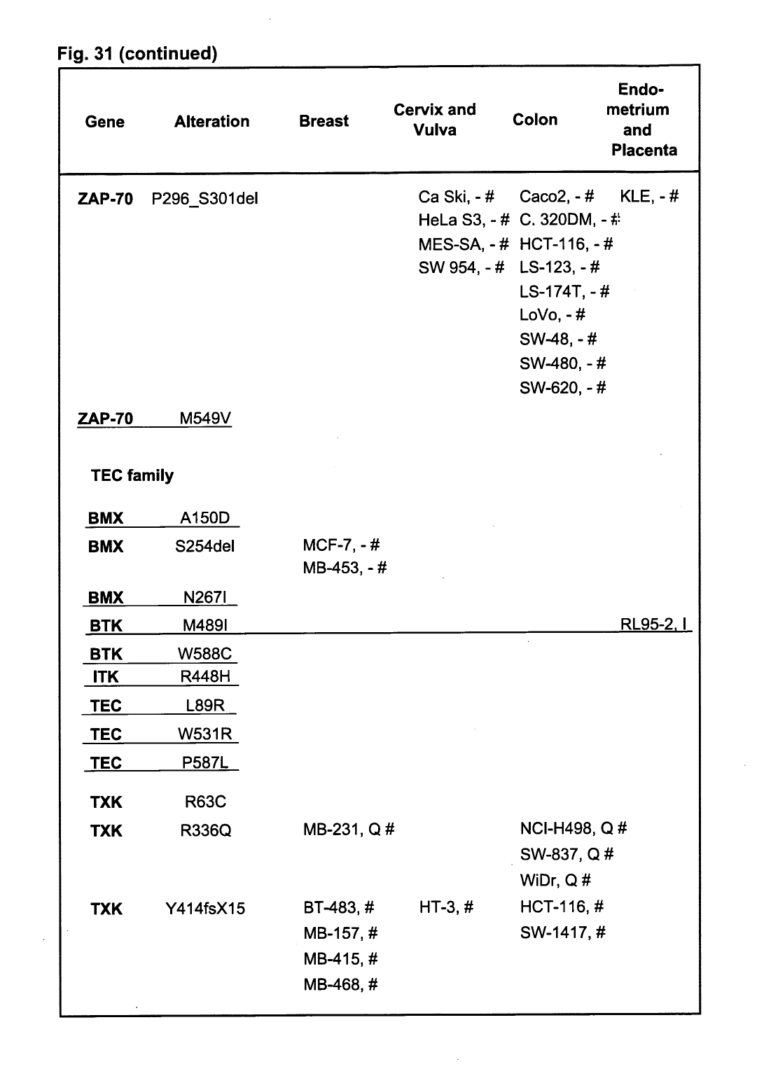

FIG. 2 depicts a characterization of protein tyrosine kinase genes with regard to genetic alterations detected in the transcripts of 276 tumor cell lines and control samples. As exemplified here for FGFR4, the spectrum of identified genetic alterations and the corresponding patterns of affected tumor cell lines or control samples was determined for each tyrosine kinase gene (FIG. 31). The total sample number carrying a given sequence variant is indicated, and affected cancer cell lines are subdivided according to their tissue origin. Somatic mutations are underlined, the remaining entries are germline polymorphisms. Heterozygocity is indicated by a hash, the other samples are homozygous carriers of the respective alteration.

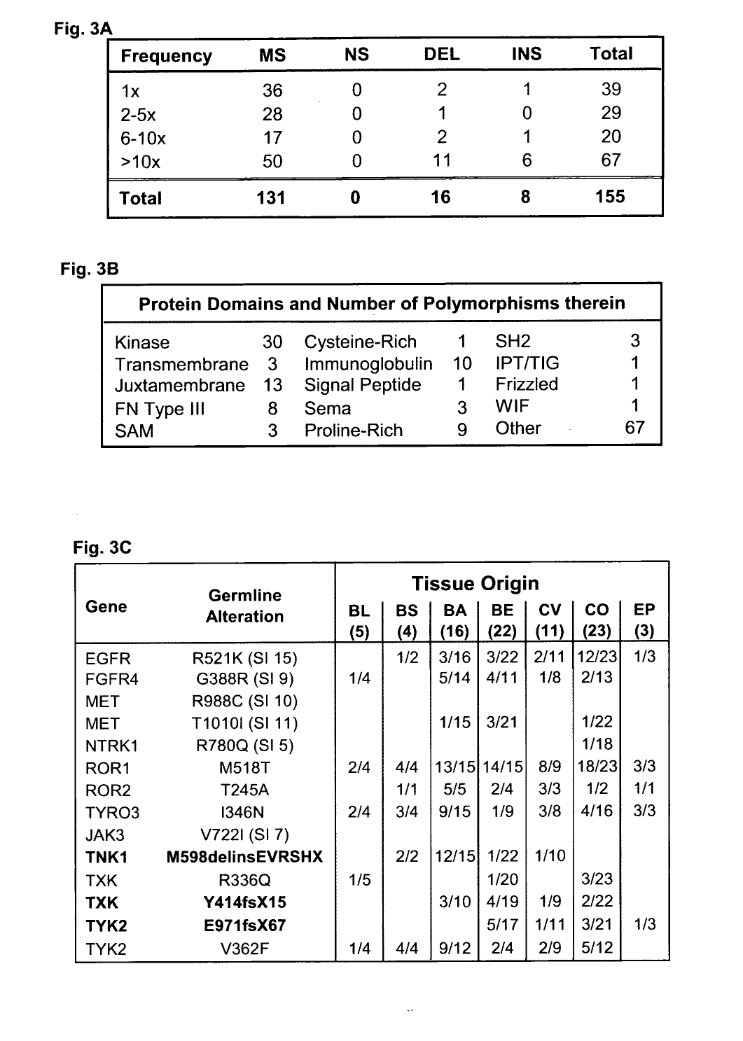

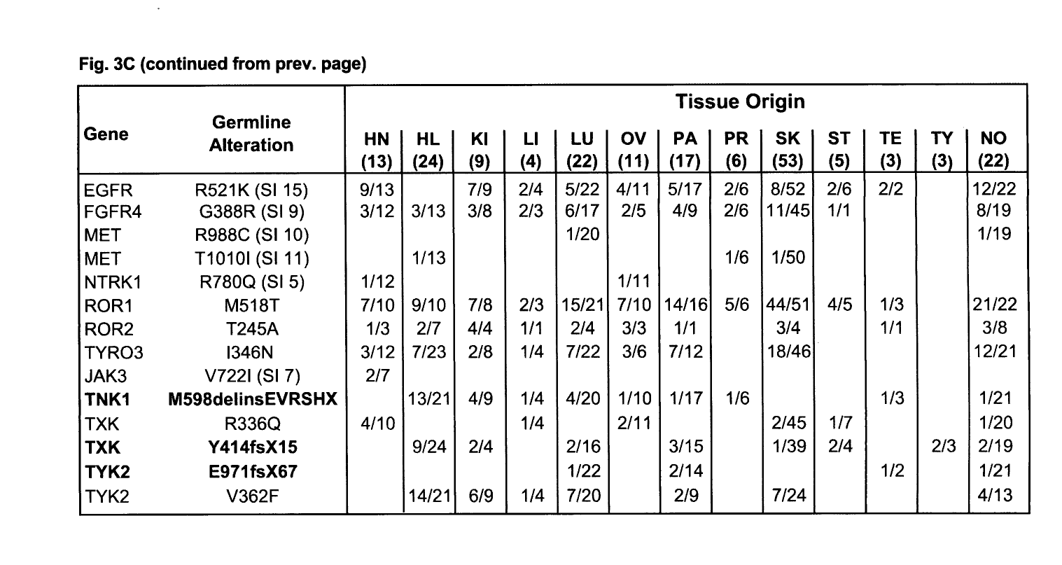

FIG. 3 shows the distributions of non-synonymous polymorphisms identified in the TKT of 276 cancer cell lines and control samples. FIG. 3A: rates of germline alterations. The rates of missense (MS) or nonsense (NS) substitutions, deletions (DEL) and insertions (INS) subdivided into four frequency categories (1, 2-5, 6-10 or more than 10 affected samples) are summarized. FIG. 3B: domain localization of identified polymorphisms. The number of polymorphisms detected in distinct domains or other protein regions is indicated. FIG. 3C: tissue distribution of germline variations. The tissue distribution (BL: bladder; BS: bone and soft tissue; BA: brain; BE: breast; CV: cervix and vulva; CO: colon; EP: endometrium and placenta; HN: head and neck; HL: hematopoietic and lymphoid system; KI: kidney; LI: liver; LU: lung; OV: ovary; PA: pancreas; PR: prostate; SK: skin; ST: stomach; TE: testes; TY: thyroid, NO: normal control samples) was determined for all polymorphisms (FIG. 33) and presented here for those described in the text. Paired numerals indicate the number of carriers of the indicated variant as a subset of all cell lines with the same tissue origin that express the corresponding gene regardless of its genotype. Novel germline alterations are highlighted in bold type, parenthesized numbers refer to the following references that associate respective polymorphisms with cancer: 1.: Ullrich, A., et al., Nature, 313: 756-761, 1985; 2: Galland, F., et al., Oncogene, 8: 1233-1240, 1993; 3: Schmidt, L. S., et al. J Urol, 172: 1256-1261, 2004; 4: Gimm, O., et al., J Clin Endocrinol Metab, 84: 2784-2787, 1999; 5: Greco, A., et al. Am J Hum Genet, 64: 1207-1210, 1999; 6: Walter, J. W., et al. Genes Chromosomes Cancer, 33: 295-303, 2002; 7: Walters, D. K., et al., Cancer Cell, 10: 65-75, 2006; 8: Xie, D., et al. J Natl Cancer Inst., 92: 412-417, 2000; 9: Bange, J., et al., Cancer Res, 62: 840-847, 2002; 10: Tjin, E. P., et al., Blood, 107: 760-768, 2006; 11: Lee, J. H., et al., Oncogene, 19: 4947-4953, 2000; 12: Bounacer, A., et al. Br J Cancer, 86: 1929-1936, 2002; 13: Sturla, L. M., et al., Br J Cancer, 89: 1276-1284, 2003; 14: Ma, P. C., et al., Cancer Res, 65: 1479-1488, 2005; 15: Moriai, T., et al., Proc Natl Acad Sci USA, 91: 10217-10221, 1994; 16: Collesi, C., et al. Mol Cell Biol, 16: 5518-5526, 1996; 17: Lynch, T. J., Bell, et al., N Engl J Med, 350: 2129-2139, 2004; 18: Huusko, P., et al., Nat Genet, 36: 979-983, 2004; 19: Beghini, A., et al. Hematol J, 3: 157-163, 2002; 20: Kong-Beltran, M., et al. Cancer Res, 66: 283-289, 2006; 21: Reindl, C., et al. Blood, 107: 3700-3707, 2006.

FIG. 4 depicts the diverging occurrence rates of polymorphisms in different tumor types and/or control samples. The frequency of homozygous (HO; dark bar) and heterozygous (HE; light bar) carriers of EGFR R521K, TYK2 V362F and TNK1 M598delinsEVRSHX was determined. Only tissue origins (for abbreviations see legend to FIG. 1, supra) with an expression of the corresponding gene in at least 10 samples have been selected for this analysis.

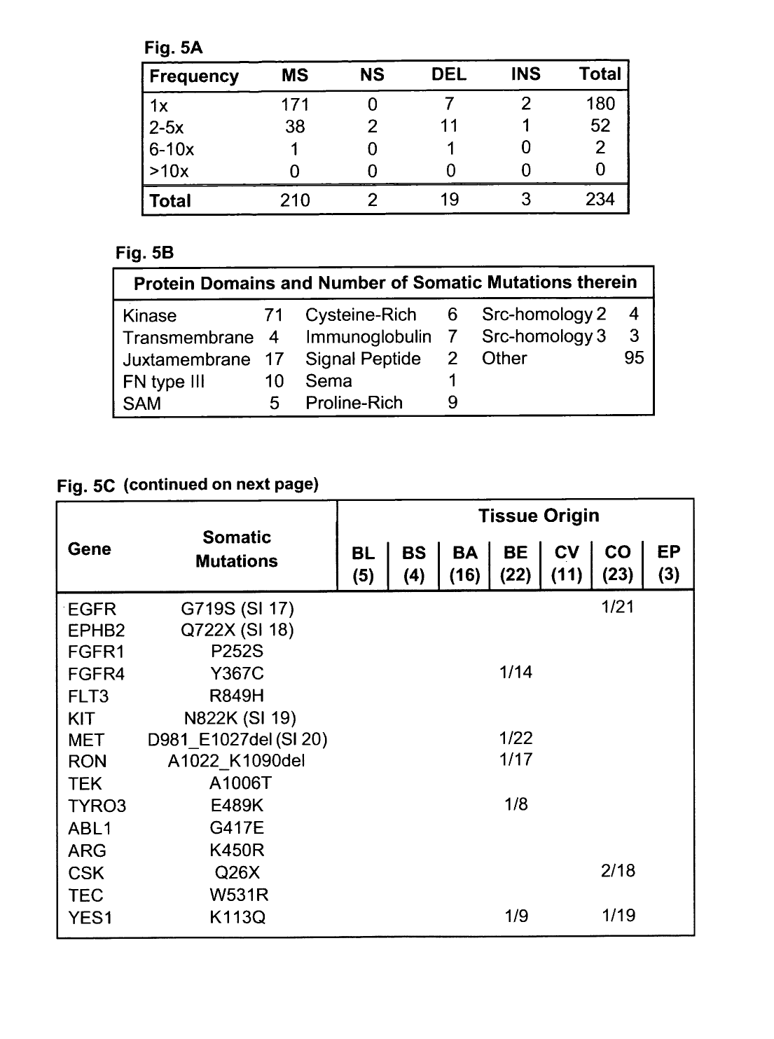

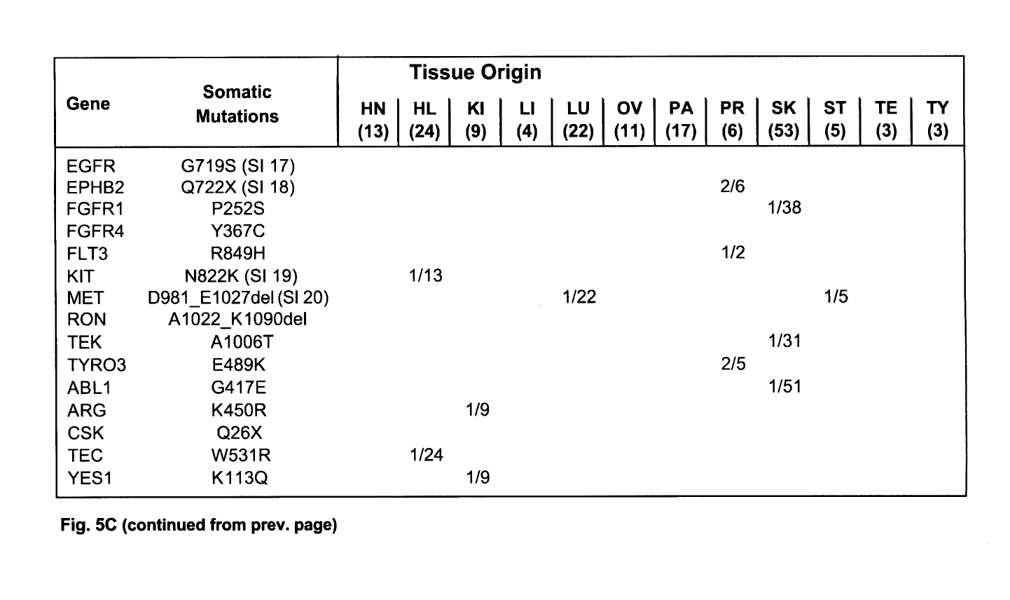

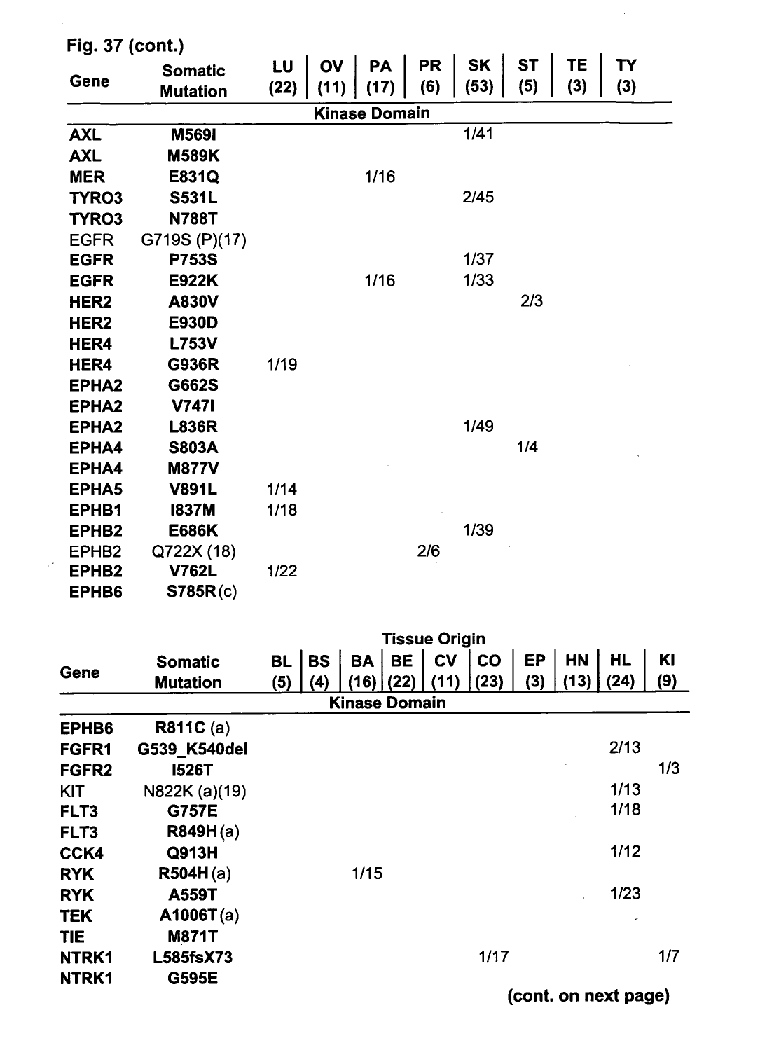

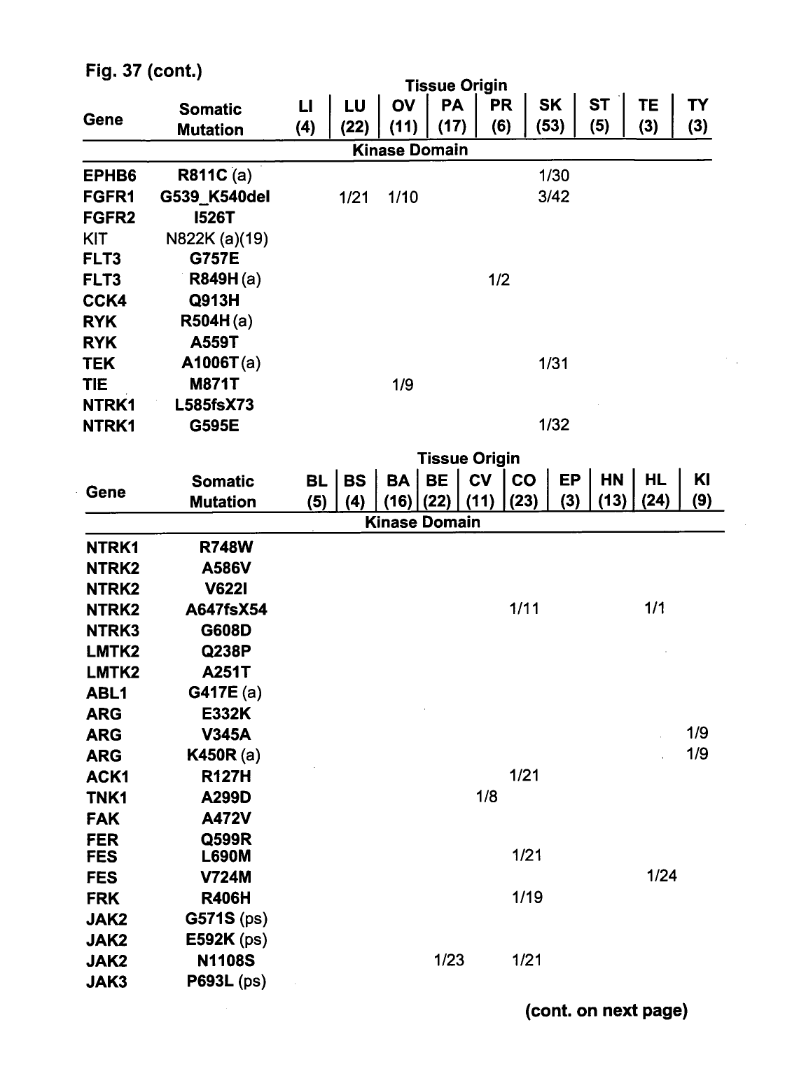

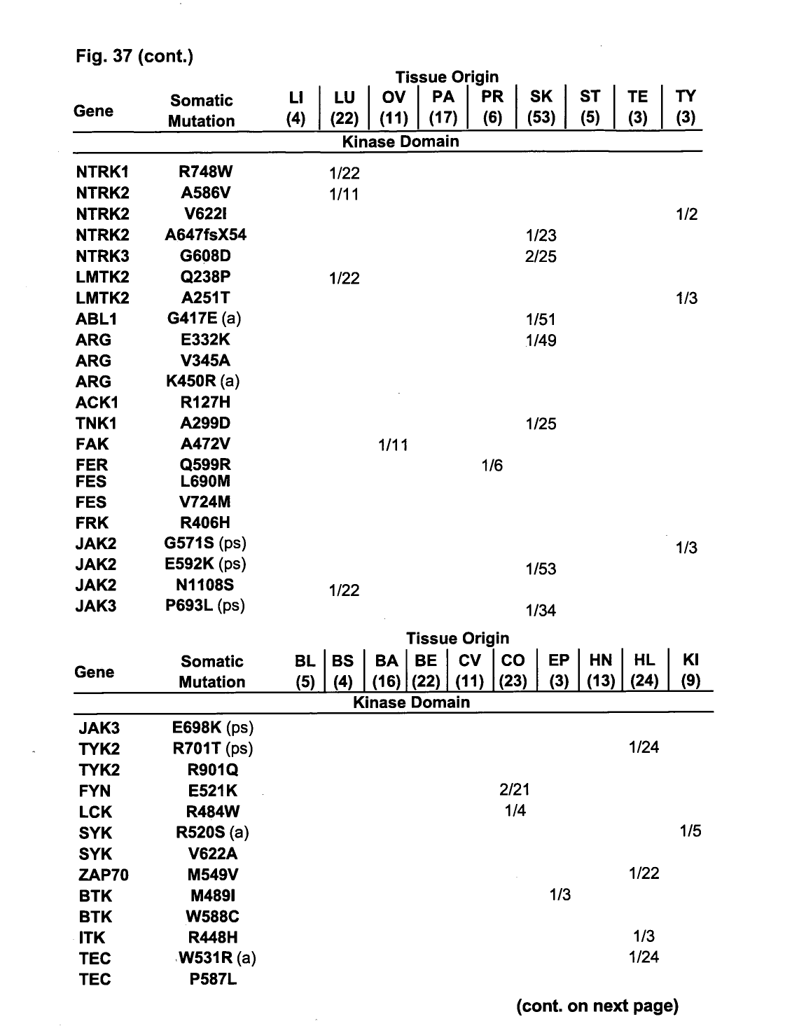

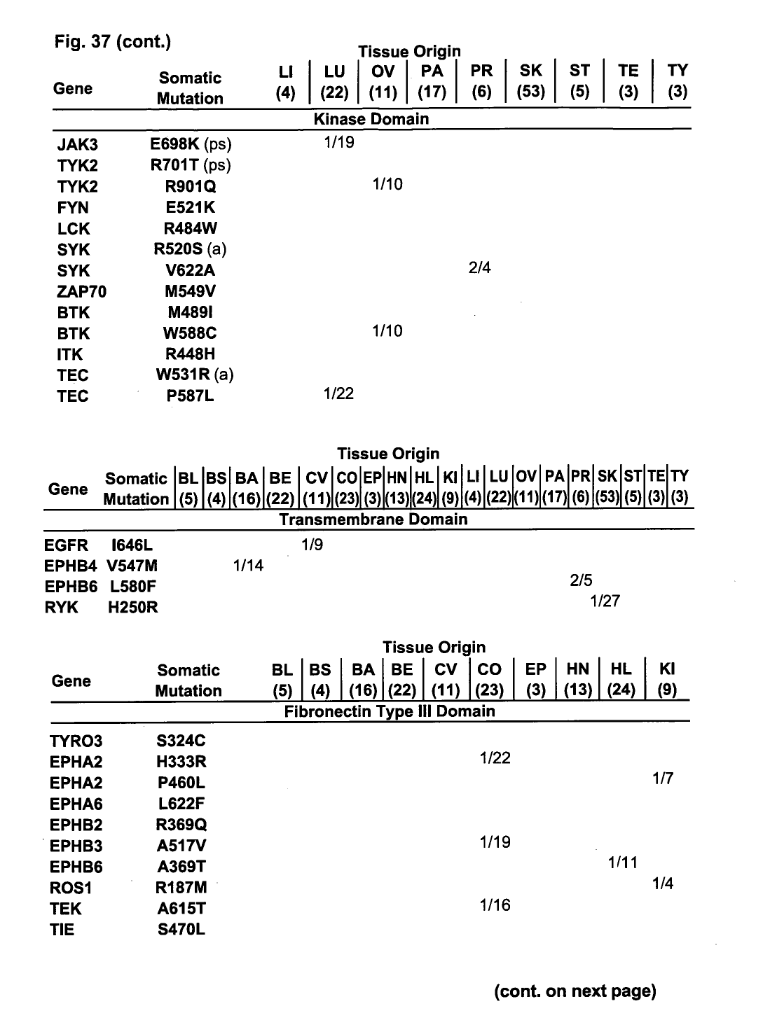

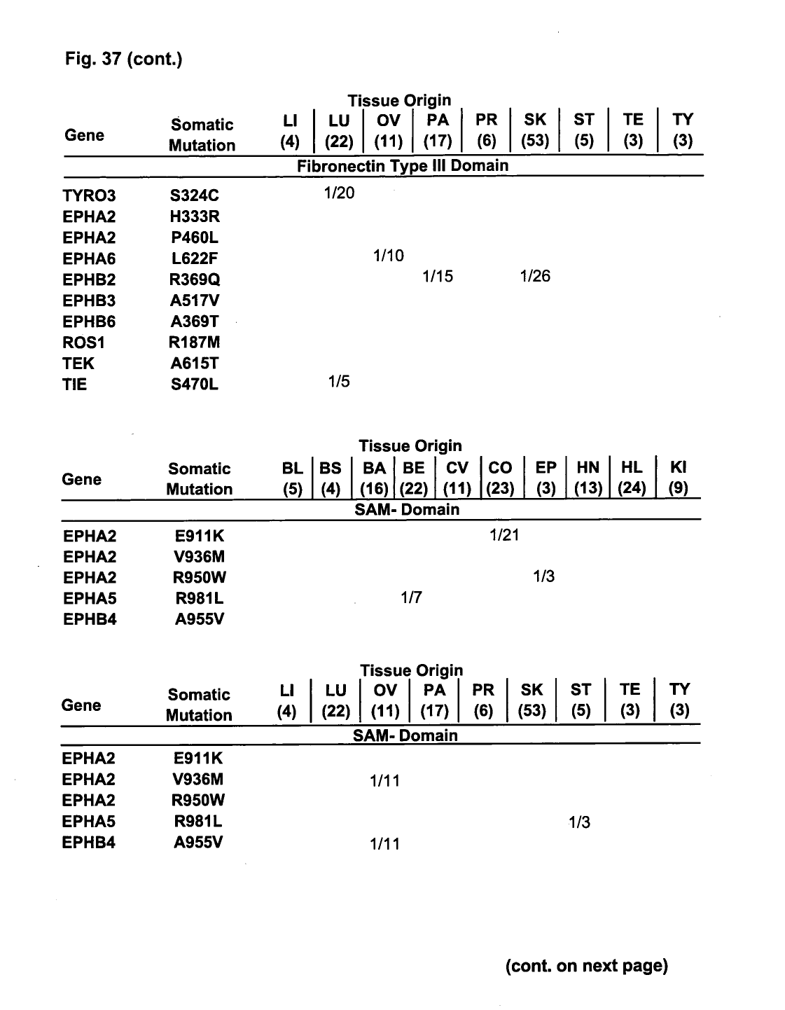

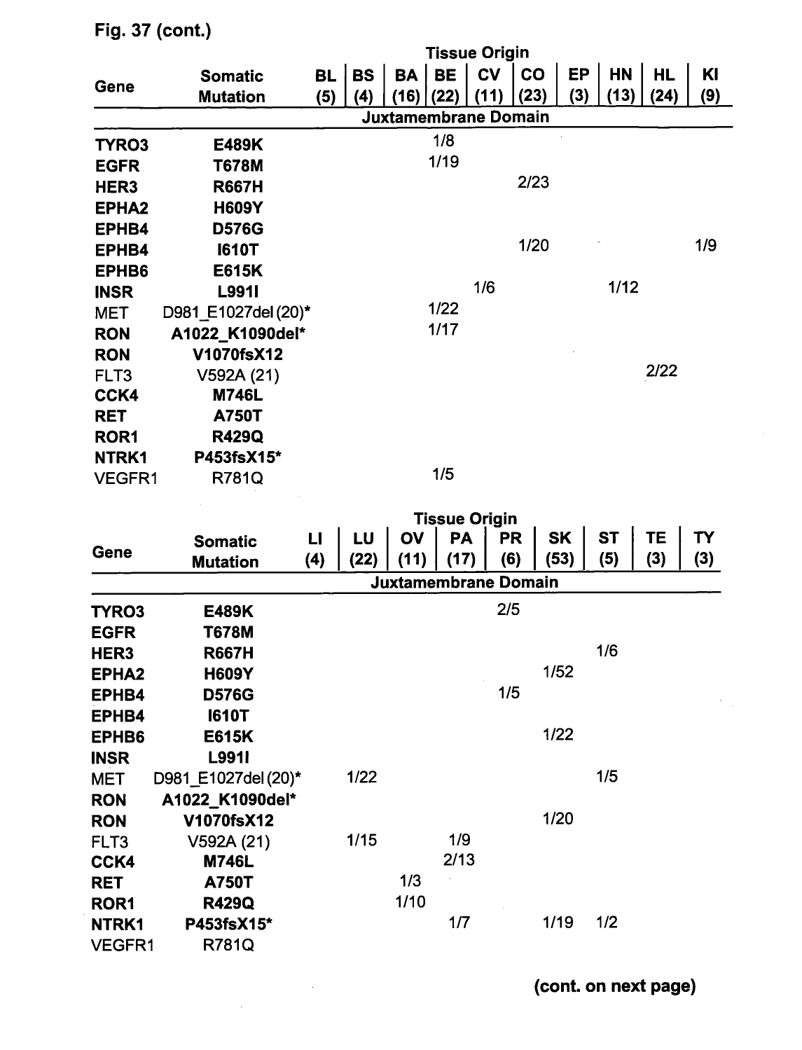

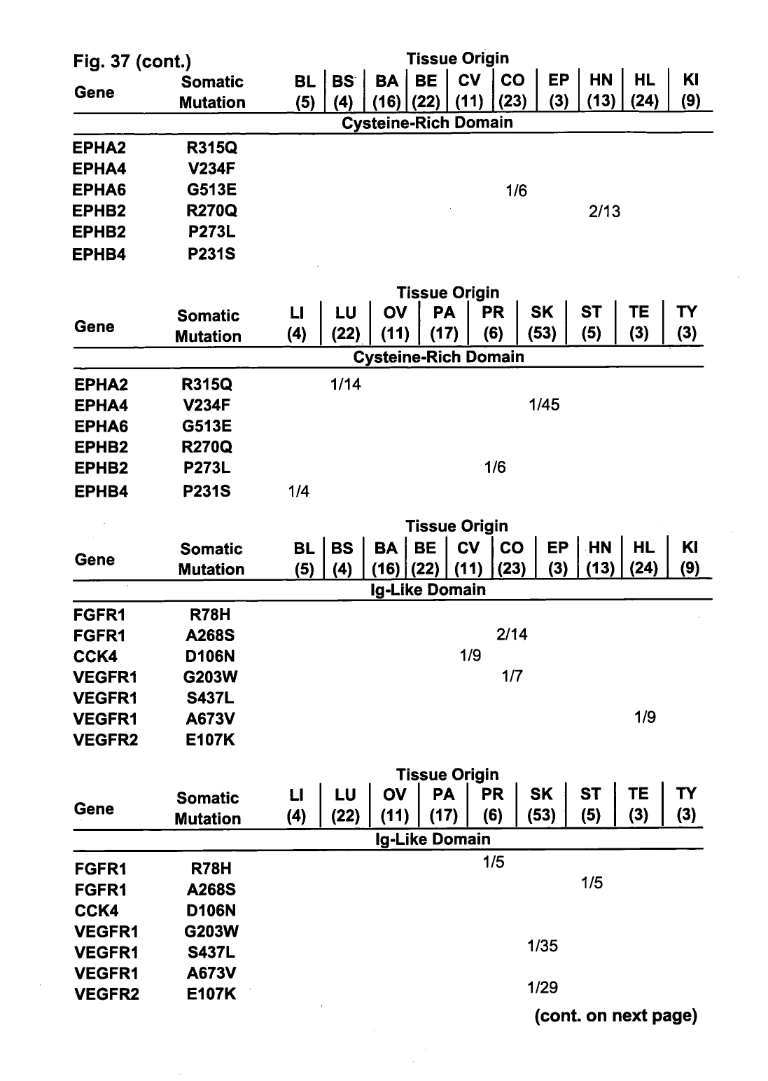

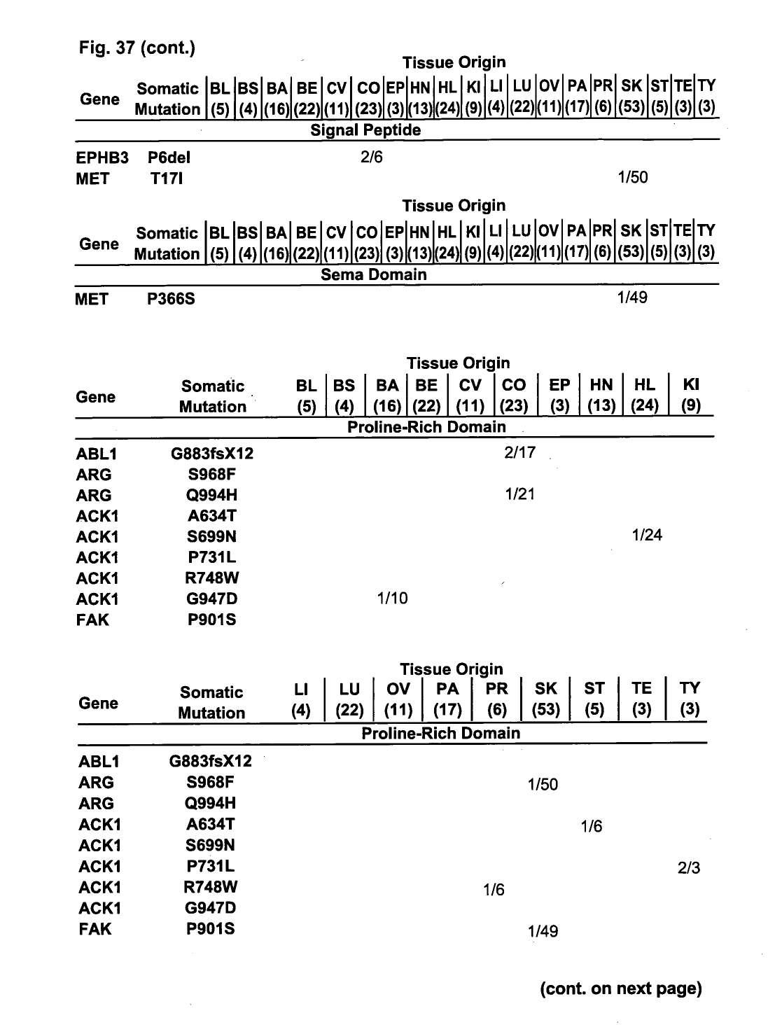

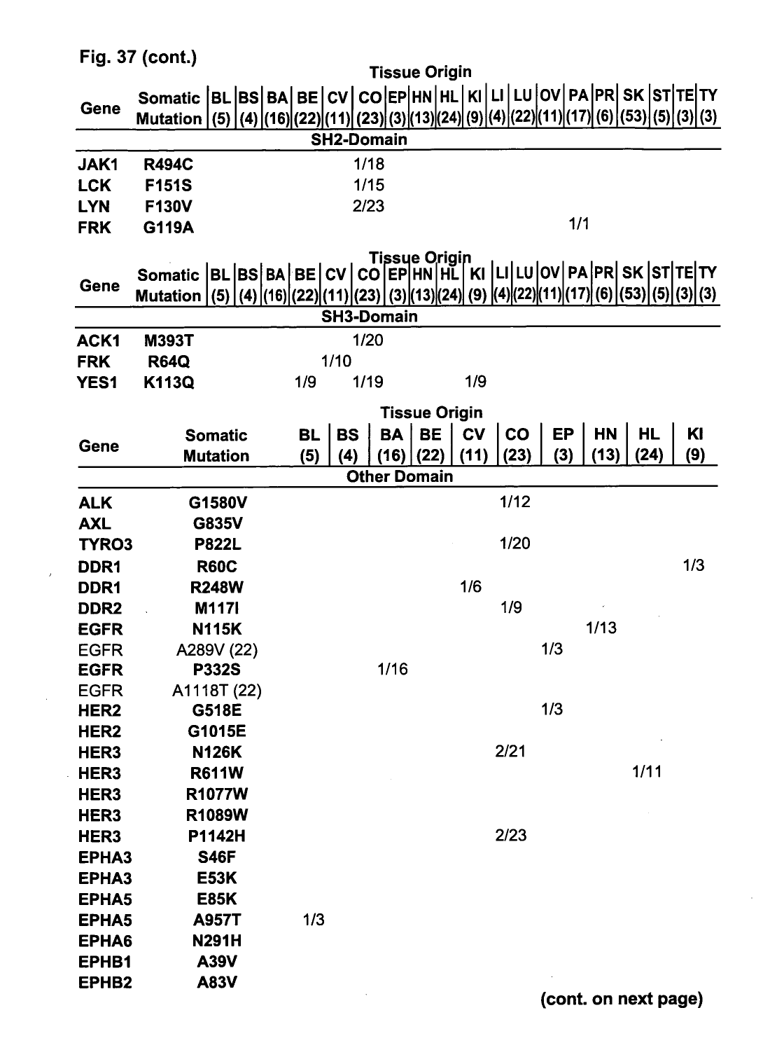

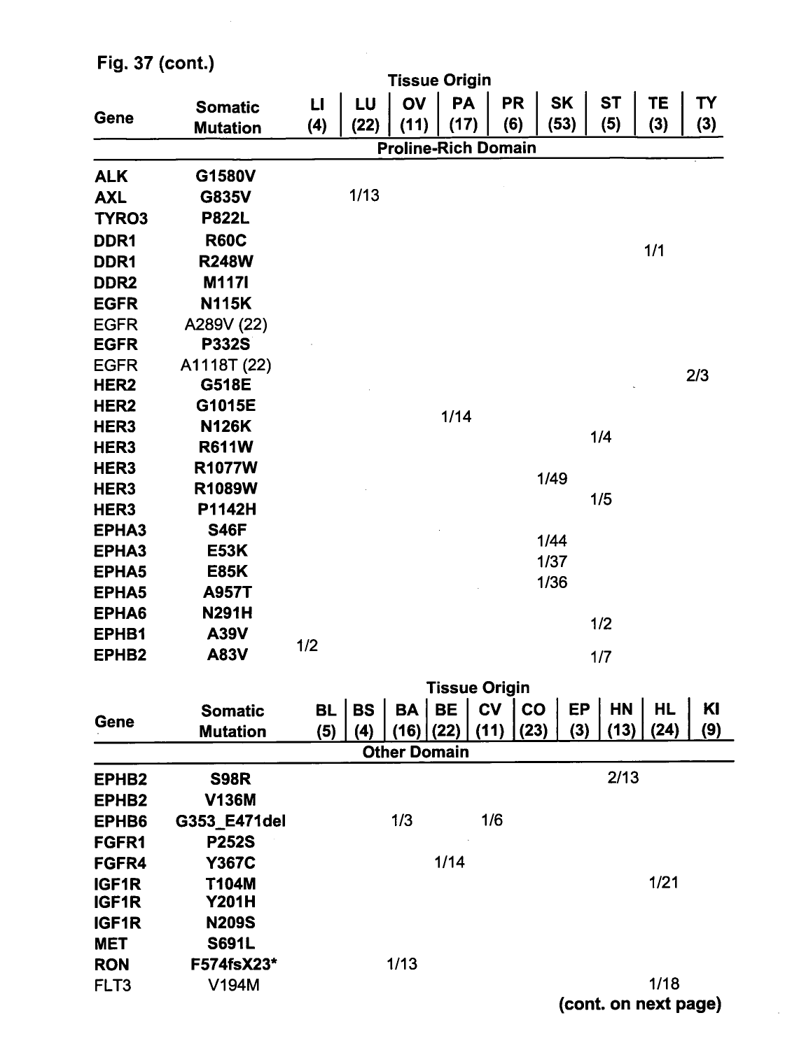

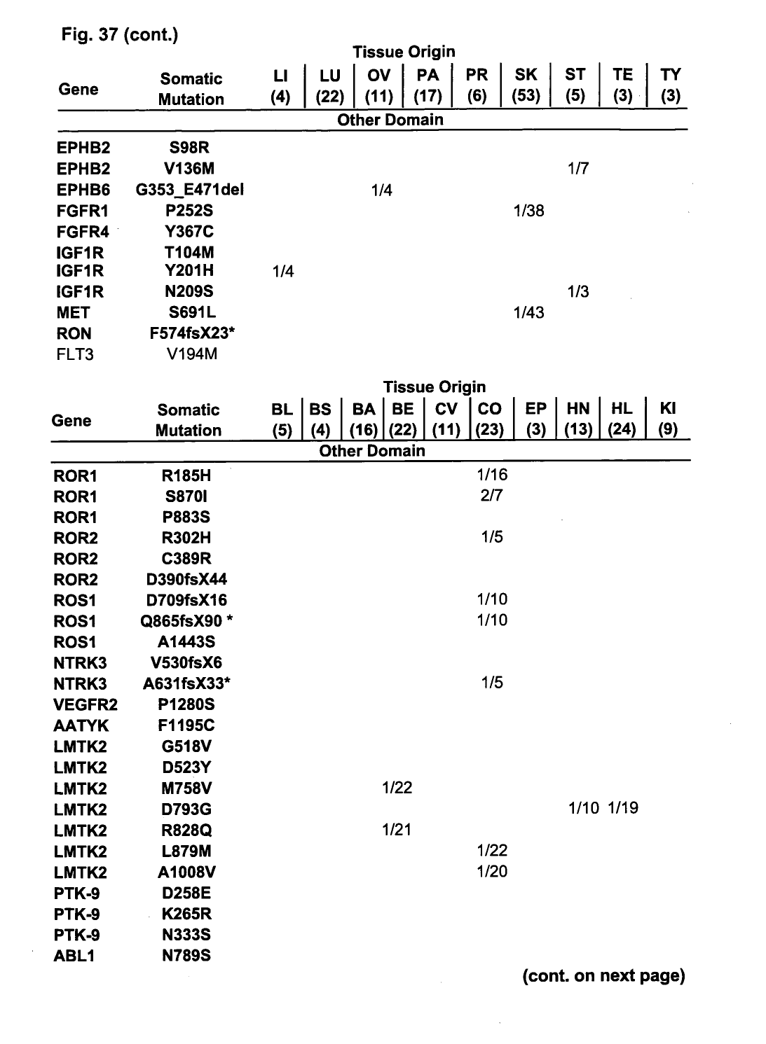

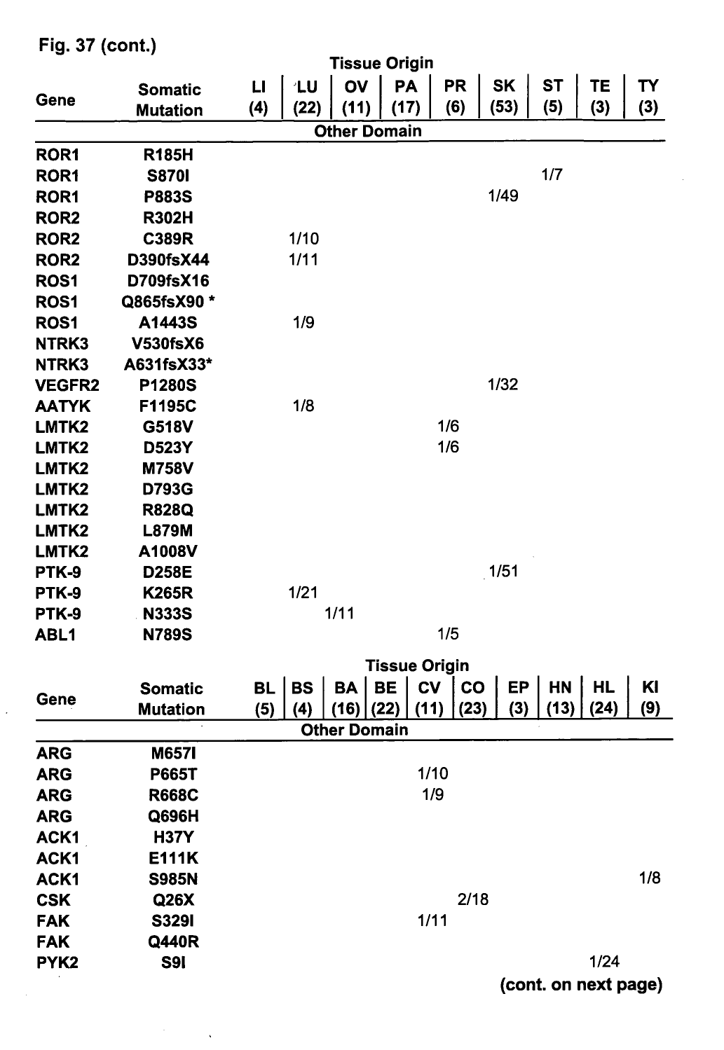

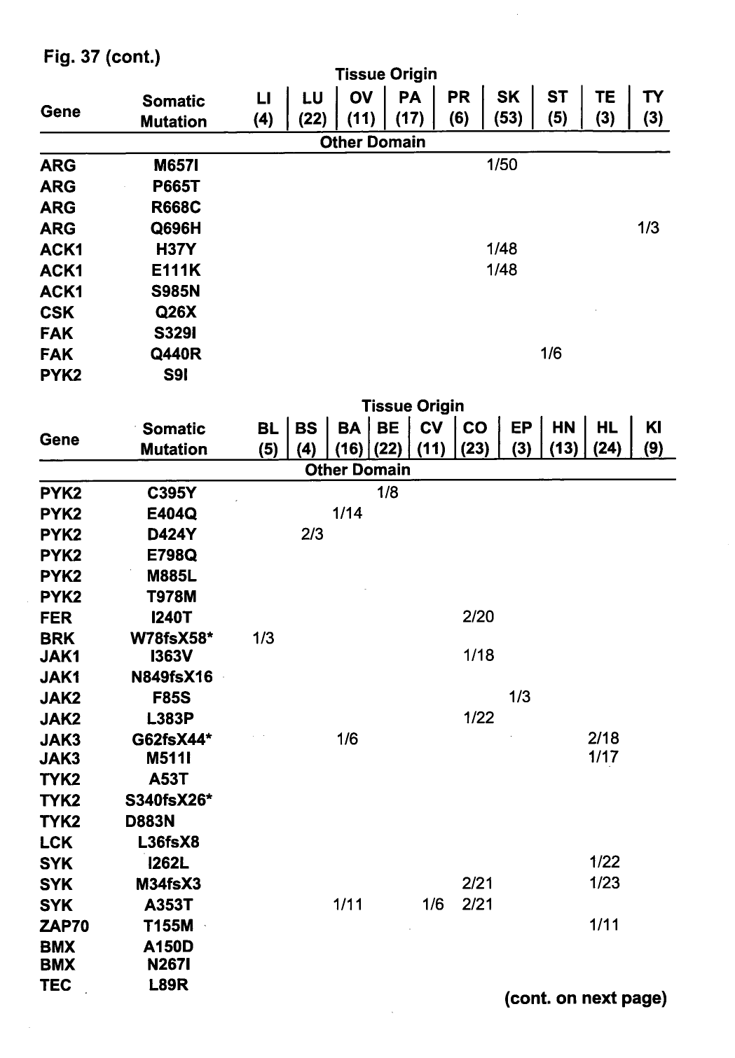

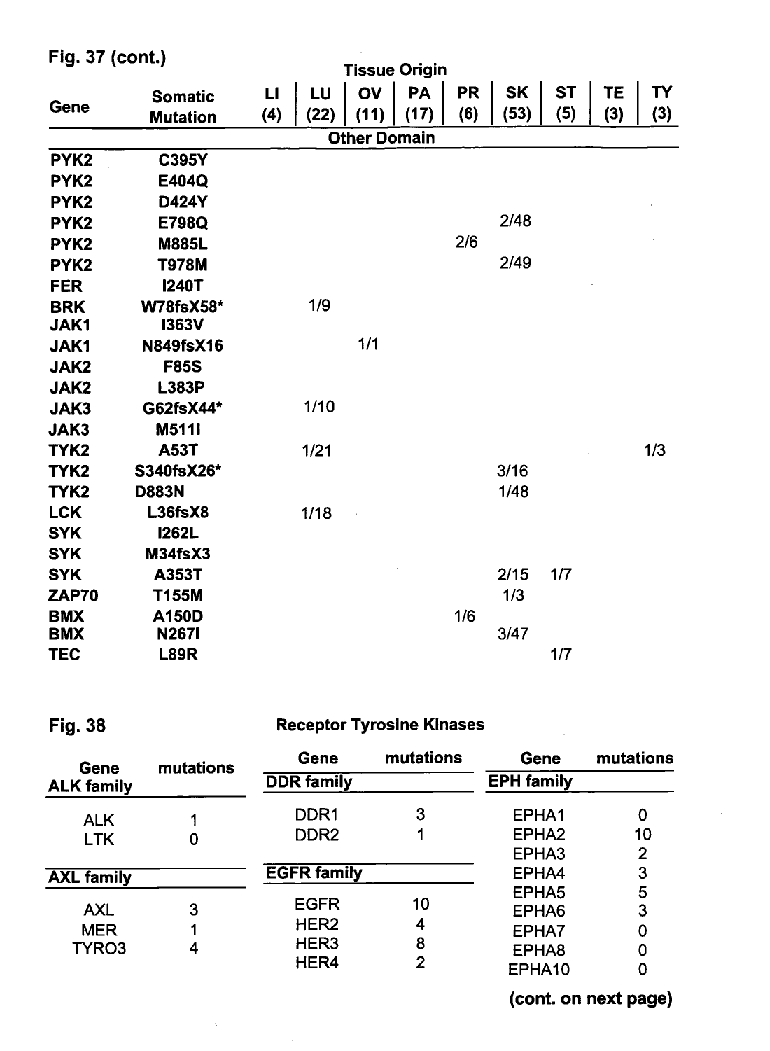

FIG. 5 depicts the distributions of non-synonymous somatic mutations identified in all transcribed PTK genes from 254 tumor cell lines. A, rates of somatic mutations. The allocation to missense (MS) or nonsense (NS) substitutions, deletions (DEL) and insertions (INS) as well as frequency categories (1, 2-5, 6-10 or more than 10 affected samples) is shown. B, domain localization of identified mutations. The localization within defined domains or other protein regions is indicated. C, tissue distribution of sporadic alterations. For each somatic mutation, the tissue distribution (see legend to FIG. 3 for abbreviations) was determined (FIG. 37) and presented here for text-related examples. Paired numerals indicate the number of mutated and expression-positive cell lines within a given tumor type. Novel somatic mutations are highlighted in bold type, numbers in parenthesis refer to references given in the legend of FIG. 3 that associate respective mutations with cancer.

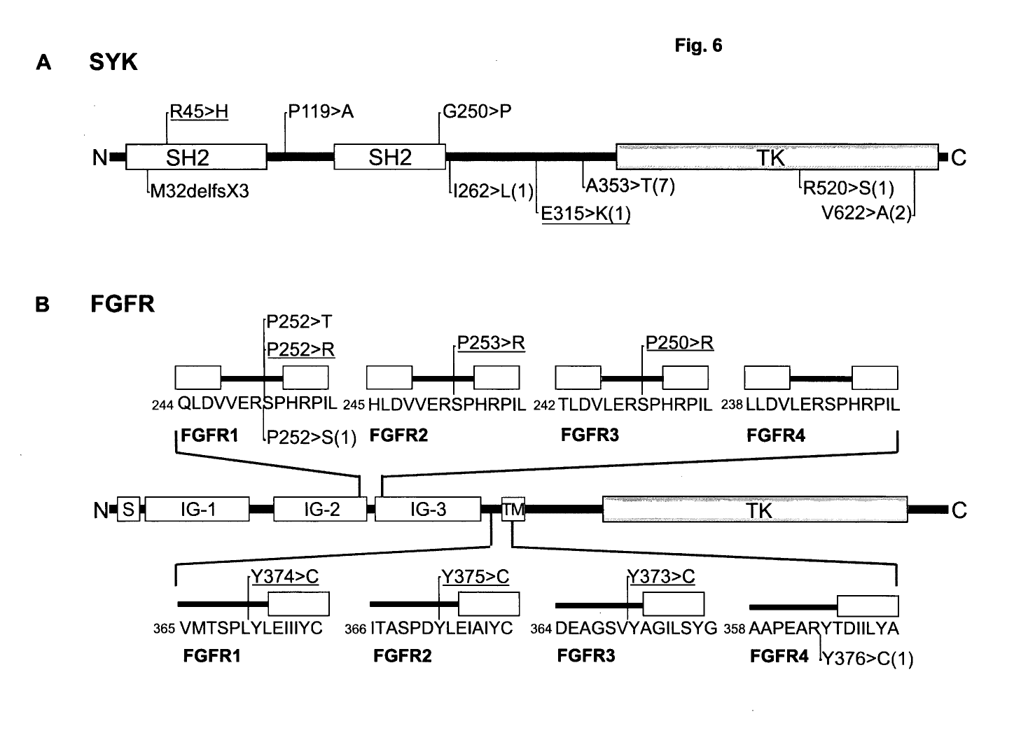

FIG. 6 is an illustration of known and novel genetic alterations in selected genes. A, SYK. The domain organization and location of genetic alterations is displayed. B, sequence comparison of FGFR1-4. For FGFR1-4, the general domain organization (middle) and sequence comparisons of the linker region connecting the IG-D2- and IG-D3-domain (top) as well as a part of the extracellular juxtamembrane region (bottom) are illustrated. Genetic alterations identified in our cell line screen are illustrated below, known sequence variants are depicted above the graphical representation of the domain structure. Polymorphisms are underlined, the remaining marked positions are somatic mutations. Numbers in parenthesis indicate the number of affected non-related cell lines. (SH2: Src Homolgy 2 Domain; TK: Tyrosine Kinase Domain; S: Signal Peptide; TM: Transmembrane Domain; IG: Immuno-globulin-Like Domain)

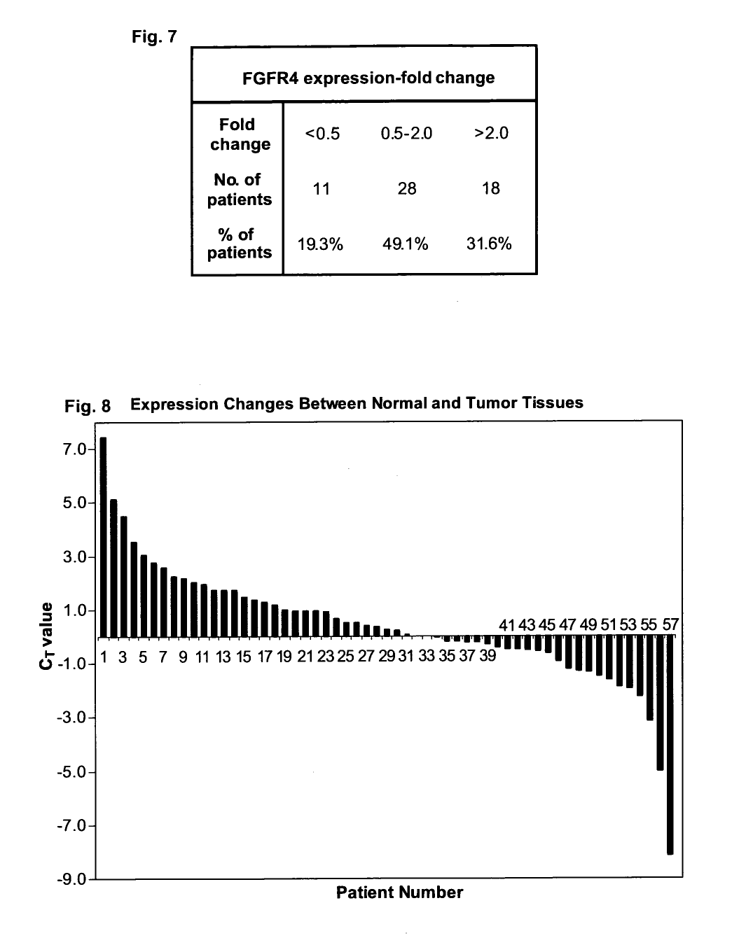

FGFR4 transcript is overexpressed (>2-fold) in ⅓ of hepatocellular carcinoma (HCC) patients (n=57) in the tumor vs. the adjacent normal tissue as determined by real-time PCR (FIG. 7, FIG. 8). The threshold cycle (Ct) value shown in FIG. 8 is scored as the cycle number where the fluorescence level crosses a predefined threshold value. The Cₜ value assigned to a particular sample thus reflects the point during the reaction at which a sufficient number of amplicons have accumulated, in that well, to be at a statistically significant point above the baseline. A lower Cₜ value accordingly reflects a higher relative gene expression.

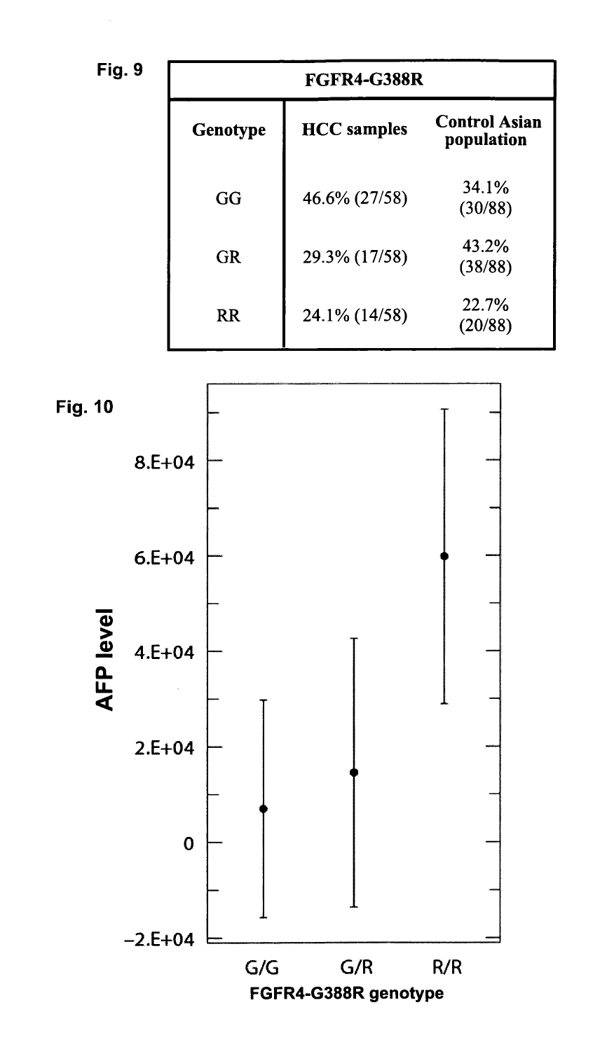

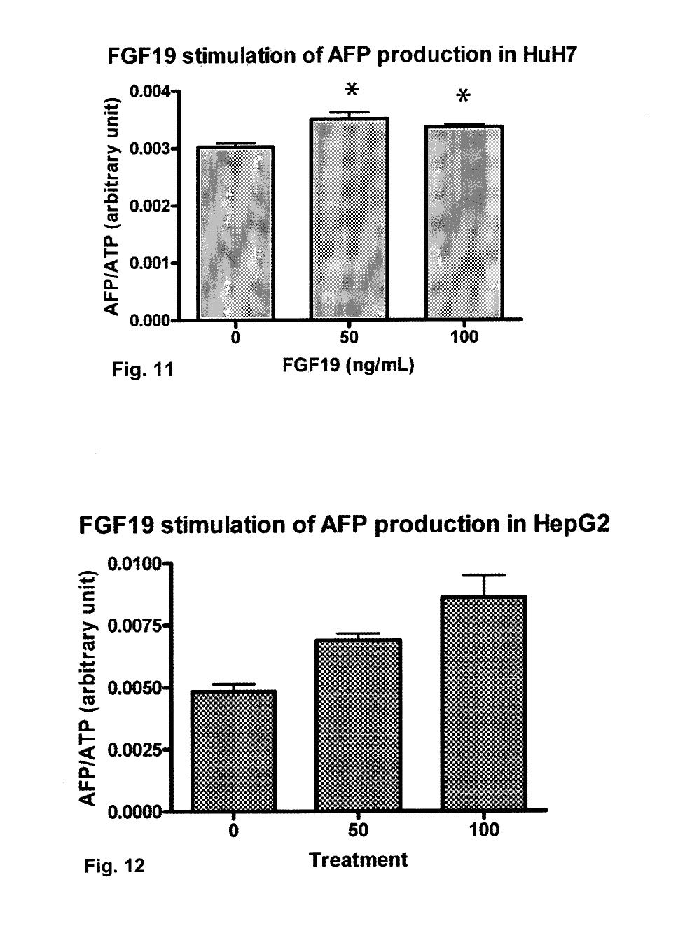

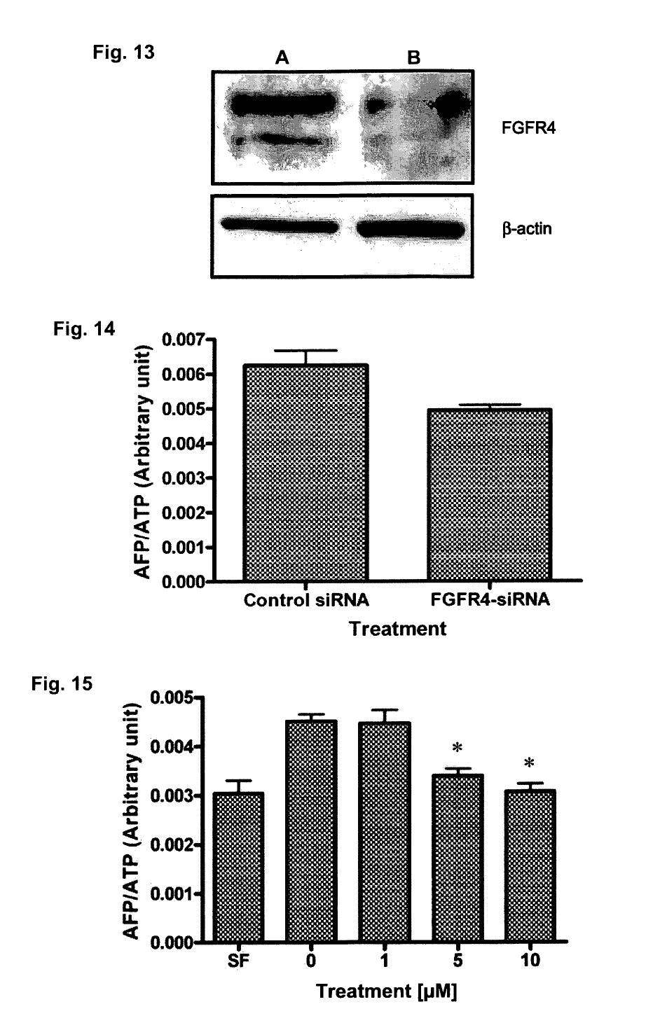

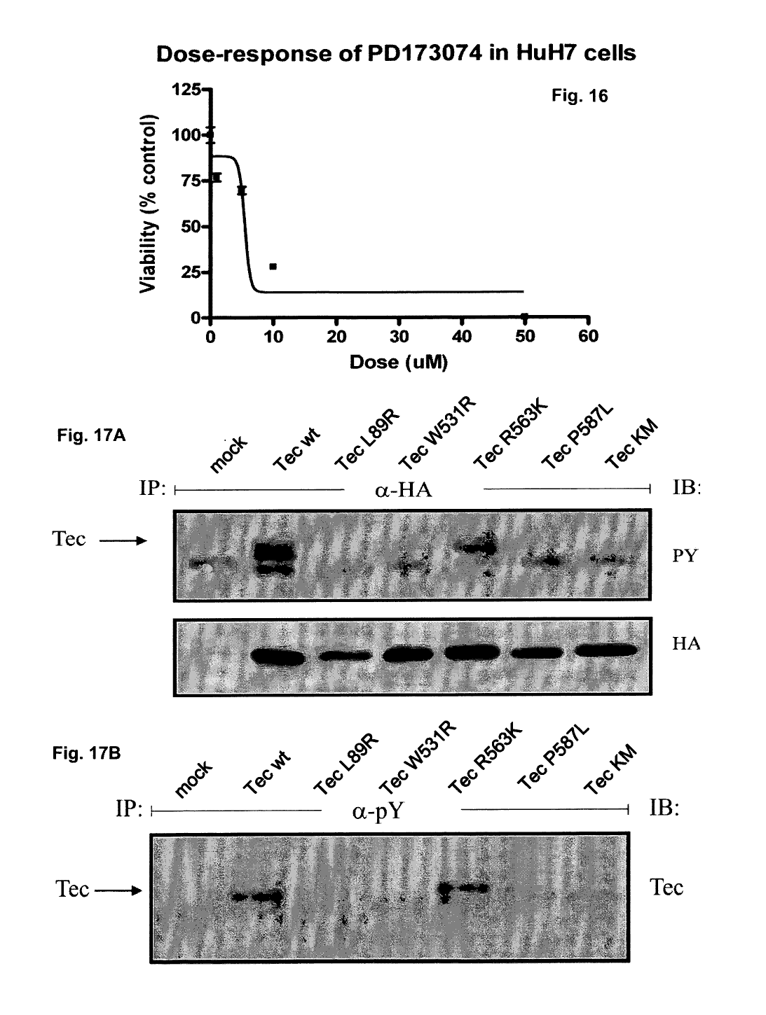

A single nucleotide polymorphism, G388R is highly represented in Asian population (including HCC patients) and the homozygous 388Arg genotype correlates with an increased alpha-fetoprotein (a diagnostic marker for HCC) secretion in the respective patients at the point of tumor resection (FIG. 9, FIG. 10). Subsequent in vitro investigations revealed that stimulation of FGFR4 in HCC cell lines using a specific ligand, FGF19, elevated AFP production by the cells (FIG. 11, FIG. 12). Gene silencing as well the administration of a commercially available non-selective FGFR inhibitor (FIG. 13, FIG. 14), PD173074 blocks AFP production (FIG. 15). Furthermore, this inhibitor exhibited exquisite anti-proliferative effect on HCC cell lines vs. non-cancerous cell line, HEK293 (data not shown, LD50>50 micromolar, see also FIG. 16). Hence, it is postulated that FGFR4 activity contributes to normal-to-tumor progression of HCC and may be a viable target for pharmacological intervention.

TEC phosphorylation was shown after SCF, GM-CSF, IL-3, IL-6 stimulation. The TEC-kinase (66 kDa) is involved in cytoskeleton reorganization by increasing actin polymerization and formation of stress fibers (phalloidin). The TEC PH domain was found to bind Vav, a specific nucleotide exchange factor for Rho, Rac and Cdc42 (Machide, M., et al., Oncogene, August 17; 11(4):619-25 (1995)). Furthermore, TEC physically associates with c-kit through a region that contain a proline-rich motif, amino terminal of the SH3 domain. (Tang, B., et al., Mol Cell Biol. 1994 December; 14(12):8432-8437). TEC activates transcription factor such as NF-KappaB, c-Jun, c-Fos, Elk1 and SRF.

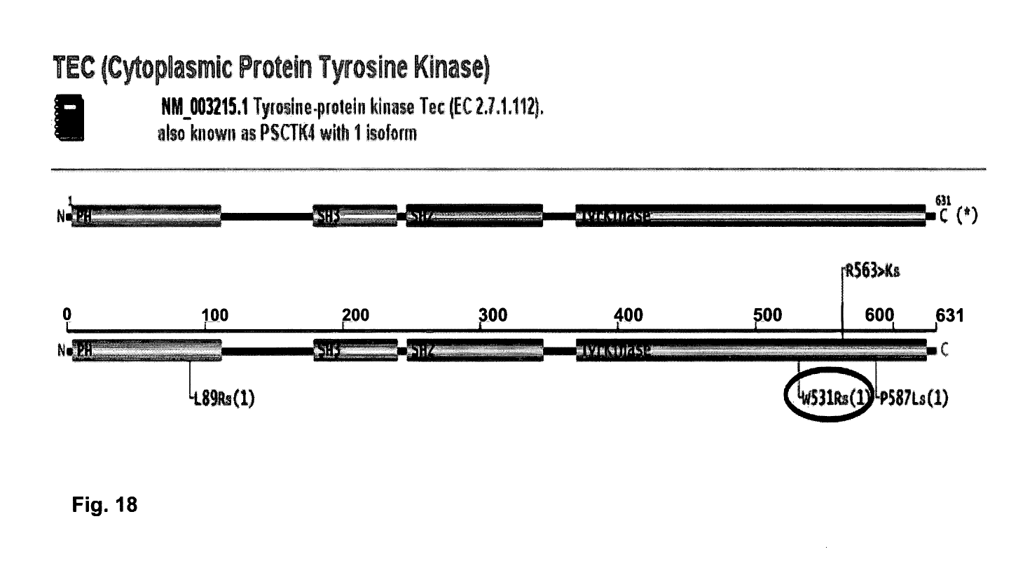

The genetic alterations identified in a cell line screen performed by the present inventors are illustrated in FIG. 18.

Mutations (all of Somatic Origin)
L89R AGS (heterozygous), mutation in pleckstrin homology domain
W531R Jurkat (homozygous), mutation in kinase domain

→P587L NCI-H661 (heterozygous), mutation in kinase domain R563K published somatic alteration

Subsequent in vitro investigation demonstrated that TEC L89R, W531R and P587L have decreased Tyrosine phosphorylation compared to TEC wt (FIG. 17). In addition, these somatic alterations are incapable of activating MAPK signaling (FIG. 19), c-fos (FIG. 20) and Stat3 activation (FIG. 21). Two publications support the finding that the W531R alteration abolishes kinase activity: It is shown that in Jak2 conversion of W1020 (corresponding W531 in TEC) abolished Jak2 kinase activity (Sandberg, E. M., et al., Mol. Cell. Biochem. October; 265(1-2):161-169 (2004)). Furthermore, disruption of W352 in CSK (corresponding W531 in TEC) leads to a 90% decrease in CSK activity (Lee, S., et al., Biochemistry October 8; 41(40):12107-12114 (2002).

A possible advantage of the T-cell lymphoma harboring the inactivating TEC W531R somatic mutation may be as follows: DNA damaging agents induce activation of AP-1 in T Lymphocytes and subsequent apoptosis (Kasibhatla, S., et al, Mol. Cell. 1998 March; 1(4): 543-51 (1988)). S. Kasibhatla et al. showed that treatment of Jurkat cells with Topoisomerase II inhibitors (etoposide, teniposide) or UV-B irradiation leads to activation c-fos mediated FasL expression followed by the induction of apoptosis. Etoposide (Etopophos, Eposin, Vepesid, VP-16) is used as a form of chemotherapy for various malignancies including lymphoma. Without being bound by theory it is postulated that TEC W531R may lead to resistance to etoposide treatment of T-cell Lymphoma (e.g. Jurkat cells) by decreasing Fas Ligand expression.

### Tyro3 and Mutants Expression Exhibit Enhanced Anti-Apoptotic Effects

Overexpression of Tyro3 in HEK293 confers resistance to the apoptosis upon treatment with TNFα/actinomycin-D. Furthermore, overexpression of mutants S531 L and P822L instead of wildtype Tyro3 enhanced anti-apoptotic effects. Preliminary data suggests that Tyro3 may perturb mitochondrial apoptotic signaling through the modulation of BCL2 family members. Hence, it is suggested that Tyro3 expression, and particularly the occurrence of mutants S531L and P822L may be a genetic marker for chemoresistance. Inhibitors of Tyro3 signaling may be a promising adjuvant with other chemotherapeutic agents.

### Somatic Mutation in Ack1

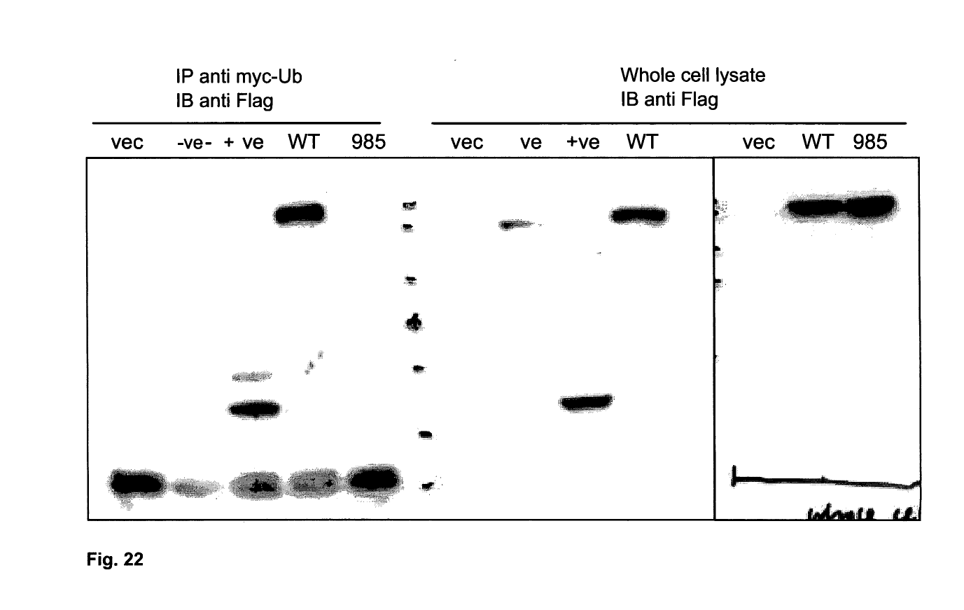

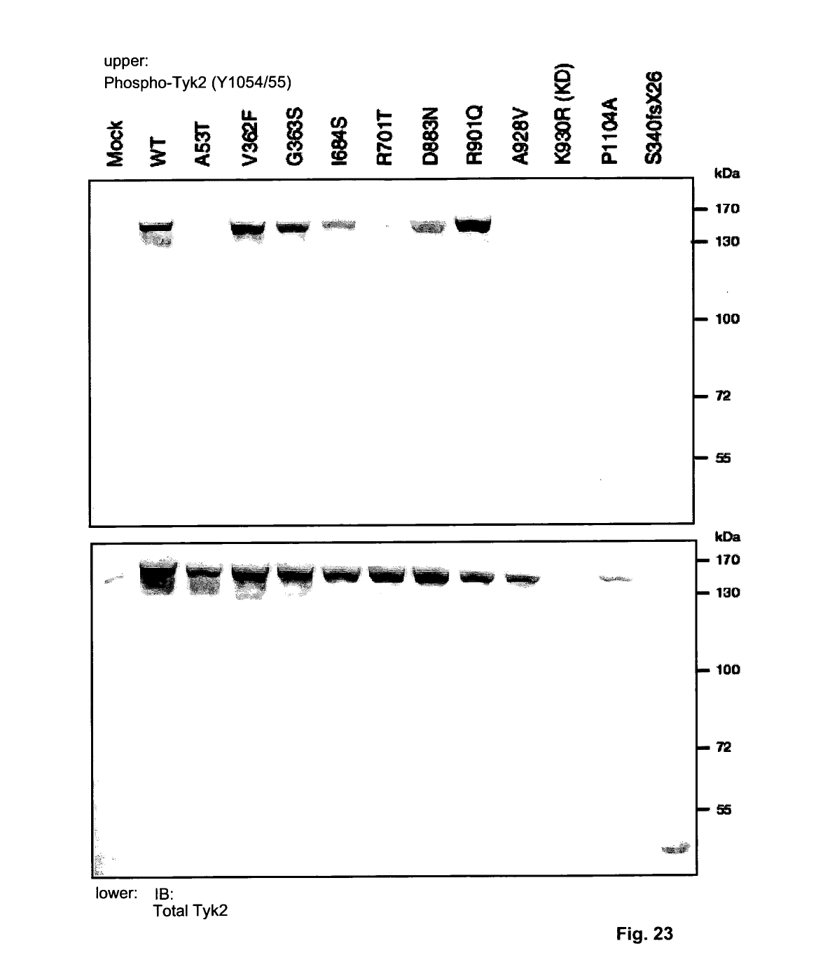

Ack1, also known as activated Cdc42-associated kinase 1, is a non receptor tyrosine kinase implicated in cancer progression. Sequencing effort initiated by Singapore OncoGenome programme has identified single base mutation that resulted in homozygous amino acid change from serine to Asparagine at amino acid 985 of kidney cancer line A498. In vitro ubiquitination assay in Hek 293 cells shows that mutation of 985 from serine to Asparagine resulted in a stable protein that is less sensitive to ubiquitination (FIG. 22).

### Alterations in TYK2 Lead to Decreased Kinase Activity.

A clearly differential occurrence of TYK2 F362 allele carriers was observed in brain—(75%) and hematopoietic/lymphoid system—(67%) derived tumor cell lines compared to control tissues (31%) or other tumor types (FIG. 4B). The under-representation of the TYK2 F362 allele in control samples indicates a tumor-promoting function with particular relevance for leukemia, melanoma, and glioma.

The skipping of entire exons in the cytoplasmic kinases TYK2 and TXK, TYK2 E971fsX67 and TXK Y414fsX15, for instance, results in frame shifts and premature translation termination in the tyrosine kinase domains, and thus most likely is associated with catalytic inactivation. The truncated TYK2 variant that lacks 206 aa of the kinase domain including the catalytic site and the activation loop may also impose a dominant negative effect on cell signals. Interestingly, Stoiber et al. reported that TYK2-deficient mice developed B and T lymphoid leukemia with higher incidence and shortened latency as a result of decreased cytotoxic capacity of TYK2−/− NK and NKT cells and thus impaired tumor surveillance (Stoiber, D., et al., J Clin Invest 114:1650-1658 (2004)). Since NK activity as part of the innate immune system mediates tumor rejection in general, the significance of TYK2 loss-of-function might not be restricted to hematopoietic malignancies, but may also be important for other cancer types. Consistent with this possibility, the inventors detected TYK2 E971fsX67 in cancer cells derived from various tissues including breast, cervix/vulva, colon, endometrium, lung, and pancreas (FIG. 3C) as well as 33 clinical breast, prostate, and kidney cancer specimens. Its occurrence in the control cell line BPH-1 suggests potential germline origin. Hence, the TYK2 E971fsX67 splice variant may also represent a prognostic marker for cancer patients and support therapeutic decision making.

### FGFR4 Y367C

The somatic mutation FGFR4 Y367C identified within the extracellular domain FGFR4 Y367C possibly augment receptor activation by receptor dimerization. The novel FGFR4 Y367C mutation was detected as a homozygous genotype in the breast cancer cell line MDA-MB-453, and the affected Y367 residue in the extracellular juxtamembrane domain is highly conserved throughout the FGFR family. Remarkably, homologous substitutions in FGFR1 (Y372C), FGFR2 (Y375C) and FGFR3 (Y373C) were shown to cause various osteogenic deficiency syndromes (Wilkie, A. O., Cytokine Growth Factor Rev 16:187-203 (2005)) through the formation of intermolecular disulfide bonds that force receptor dimerization and activation. Ligand-independent, constitutive receptor activation has been confirmed in vitro for FGFR1 Y372C (White, K. E., et al., Am J Hum Genet. 76:361-367 (2005)) and FGFR3 Y373C (d'Avis, P. Y., et al., Cell Growth Differ; 9:71-78 (1998)). Furthermore, the oncogenic potential of the FGFR3 Y373C variant has been demonstrated and was suggested to contribute to tumor progression of multiple myeloma (Chesi, M., Blood, 97:729-736 (2001)). Thus, it is most likely that Y367C as the homologous FGFR4 variant also results in basal receptor activation, which strongly indicates an important role of this mutant in cancer.

### FGFR1 P252S

FGFR1 P252S may lead to receptor activation by influencing ligand binding. The highly conserved FGFR1 P252 residue the inventors found to be heterozygously exchanged with hydrophilic serine in the melanoma cell line MeWo has previously been shown to be replaced by threonine in lung cancer (Davies, H., et al., Cancer Res; 65:7591-7595 (2005)) and arginine in patients with Pfeiffer syndrome (Muenke, M., et al., Nat Genet; 8:269-274 (1994)). The crystal structure of the homologous activating FGFR2 mutant, FGFR2P252R, revealed the formation of 3 additional hydrogen bonds with complexed fibroblast growth factor 2 (FGF2). They were predicted to increase the receptor's affinity for its specific ligand as well as to allow binding of a different set of ligands (Ibrahimi, O. A., et al., Proc Natl Acad Sci U S A; 98:7182-7187 (2001)). Since the hydroxy group of the P252-replacing serine residue in FGFR1 also has a high potential to form additional hydrogen bonds, the somatic FGFR1 P252S substitution may represent a gain-of-function mutation with analogous functional consequences as for FGFR2 P252R. This is particularly intriguing in the context of studies demonstrating that blockage of FGFR1 or bFGF function was associated with suppressed proliferation and survival of melanoma cells (Wang, Y, & Becker, D., Nat Med; 3:887-893 (1997)).

### CSK Q26X May Lead to Downregulation of Tumor Suppressor Activity

The heterozygous CSK Q26X nonsense substitution detected in the colon cancer cell lines DLD-1 and HCT-15 is consistent with reduced protein levels of this negative regulator of SRC-family kinases that were reported for ˜60% of human colon cancer cases with elevated SRC activity (Rengifo-Cam, W., et al., Oncogene; 23:289-297 (2004)). These data indicate a significant role of CSK nonsense mutations in the development and/or progression of colon carcinoma and therefore strongly suggest the inclusion of SRC kinase inhibitors in the therapeutic regimen of this prevalent malignancy.

### EXAMPLES

### Samples

Samples of primary invasive breast carcinomas were obtained from the archives of the Department of Pathology of the Technical University of Munich, Germany (Prof H. Hoefler) and the Department of Oncology of the University of Chieti, Italy (Dr. S. Iacobelli). Kidney tissue materials of tumors and healthy tissue as well as prostate cancer tissue were obtained from the Urology Department of the Klinikum Darmstadt, Germany (Prof. S. Peter). 14 cDNAs of normal tissue (spleen, testes, ovary, kidney, skeletal muscle, colon, prostate, bladder, cervix, pancreas, liver, brain, lung, gastric) derived from different individuals were purchased from Ambion.

Genomic DNA of 90 blood samples derived from non-cancer patients was purchased from Conch Institute for Medical Research (Camden, N.J., USA).

cDNA Synthesis

Total RNA was isolated according to the method described by Puissant and Houdebine.

Cancer cell lines were cultured according to conditions by the American Type Culture Collection (ATTC, http://www.atcc.org).

After homogenization of the cultured human cancer cells (80% confluency) or the primary tissue in a denaturing solution (4M guanidine thiocyanate, 25 mM sodium citrate, 0.5% Sarkosyl, 0.1M β-mercaptoethanol, 10 mM EDTA), the homogenate was sequentially mixed with 2M sodium acetate (pH 4.0), saturated phenol and finally with chloroform. The mixture was centrifuged and the upper phase was isopropanol precipitated, resuspended in denaturing solution and again reprecipitated with isopropanol. Following ethanol washing the pellet was resuspended in H₂O and incubated at 65° C. for 5 min. The quality of the total RNA was tested by gel electrophoresis.

For the extraction of poly(A)⁺RNA, total RNA was denatured at 70° C. (5 min) and applied to a oligo-dT-cellulose column along with a washing buffer (10 mM Tris/HCl pH7.4, 0.5M NaCl, 1 mM EDTA, 0.5% SDS). Following several washing steps the poly(A)⁺RNA was eluted (10 mM Tris/HCl pH7.4, 1 mM EDTA, 0.5% SDS) and precipitated with ethanol.

The conversion of the poly(A)⁺RNA into the complementary DNA (cDNA) was performed using the AMV reverse transcriptase (Promega AMV-RT) and oligo(dT) polymers and oligonucleotides (dNTP). After the synthesis the cDNA was purified using Qiagen PCR purification columns and eluted in 50 μl.

### PCR and Sequencing

For each cell line and control sample, whole cell cDNA was prepared and used for the amplification and direct sequencing of the complete PTK coding region. Primers for PCR amplification (and sequencing) were designed using Primer3 program (http://www-genome.wi.mit.edu/cgi-bin/primer/primer3_www.cgi), and were synthesized by Proligo (Singapore). PCR amplification was performed on cDNA from 265 early passage cell lines, placenta and 14 normal tissues (Ambion). PCR optimization was done in the first step with a cDNA pool of different cell lines, second step with 6/8 different individual cell lines, before being dispensed into a 96-well culture plate. Direct sequencing was done using a 96 capillary automated sequencing apparatus (ABI 3730XL).

### Analysis of Mutations

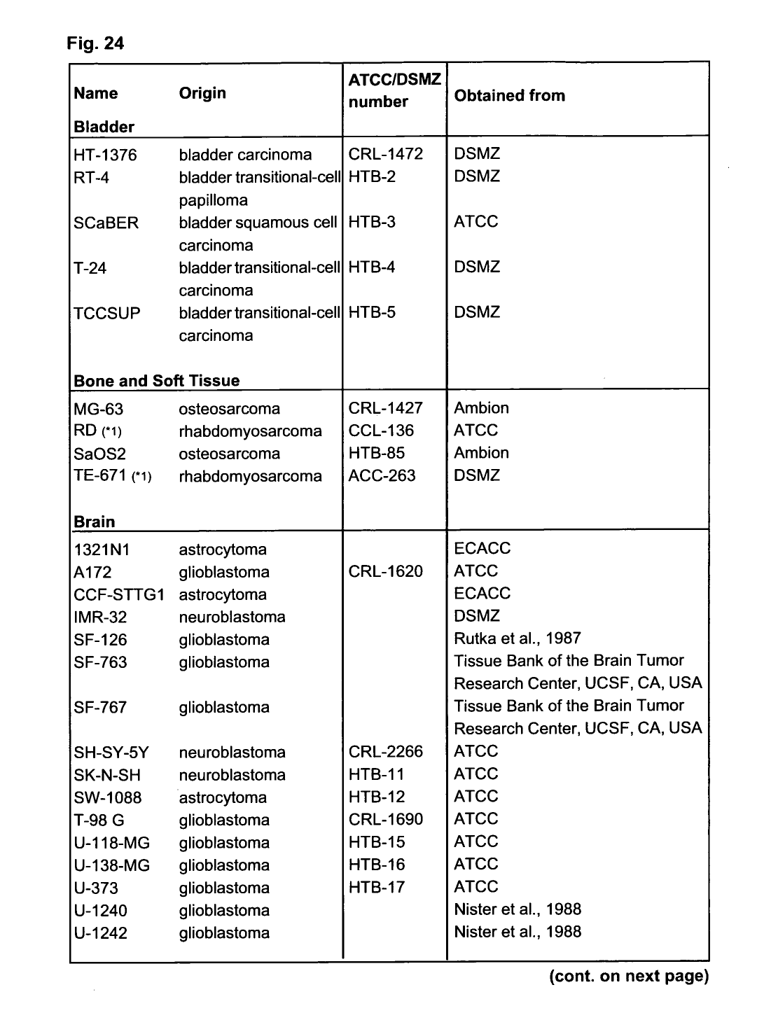

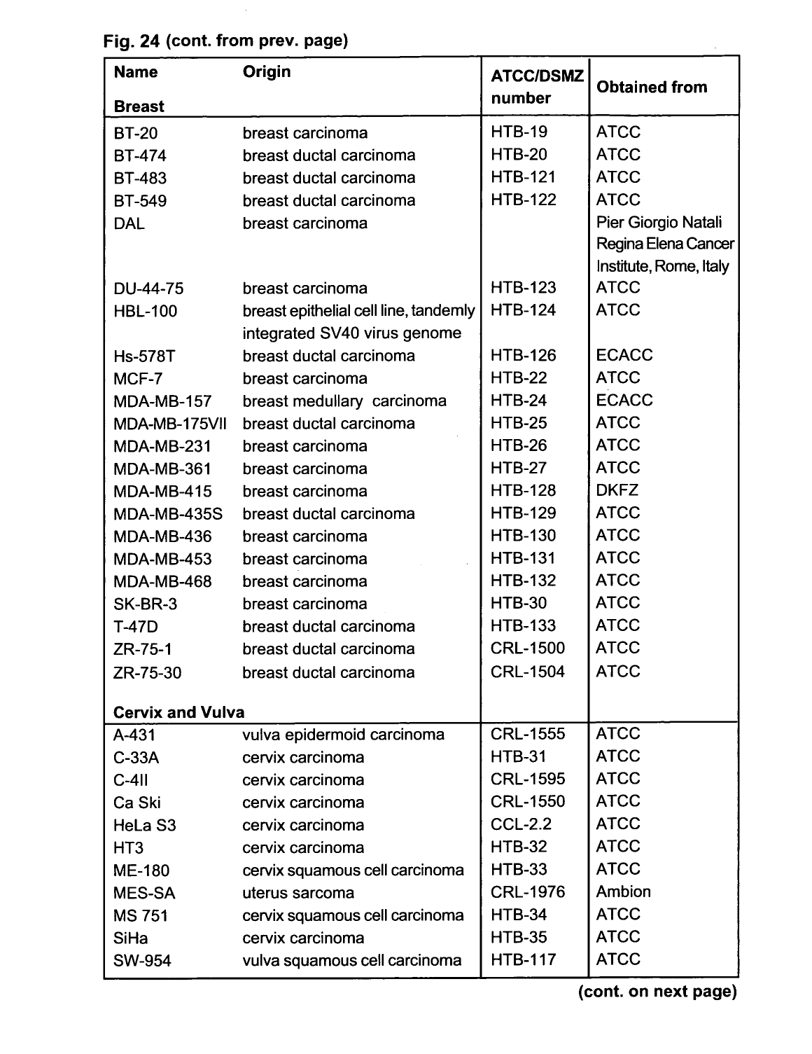

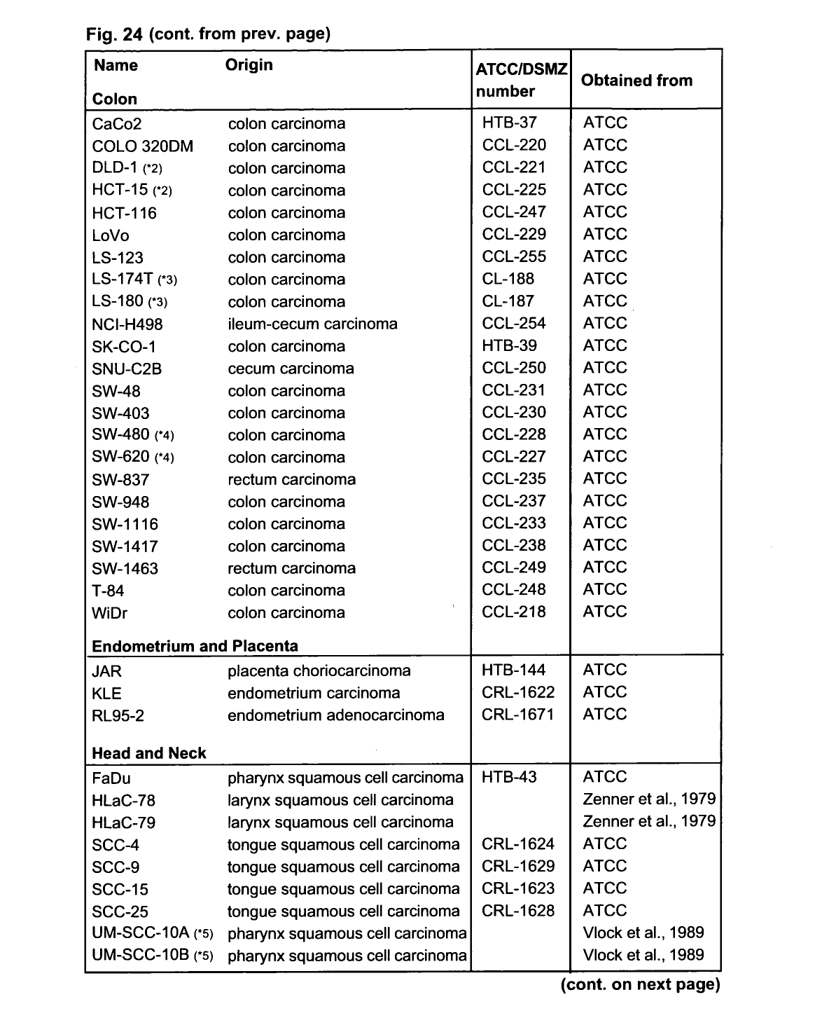

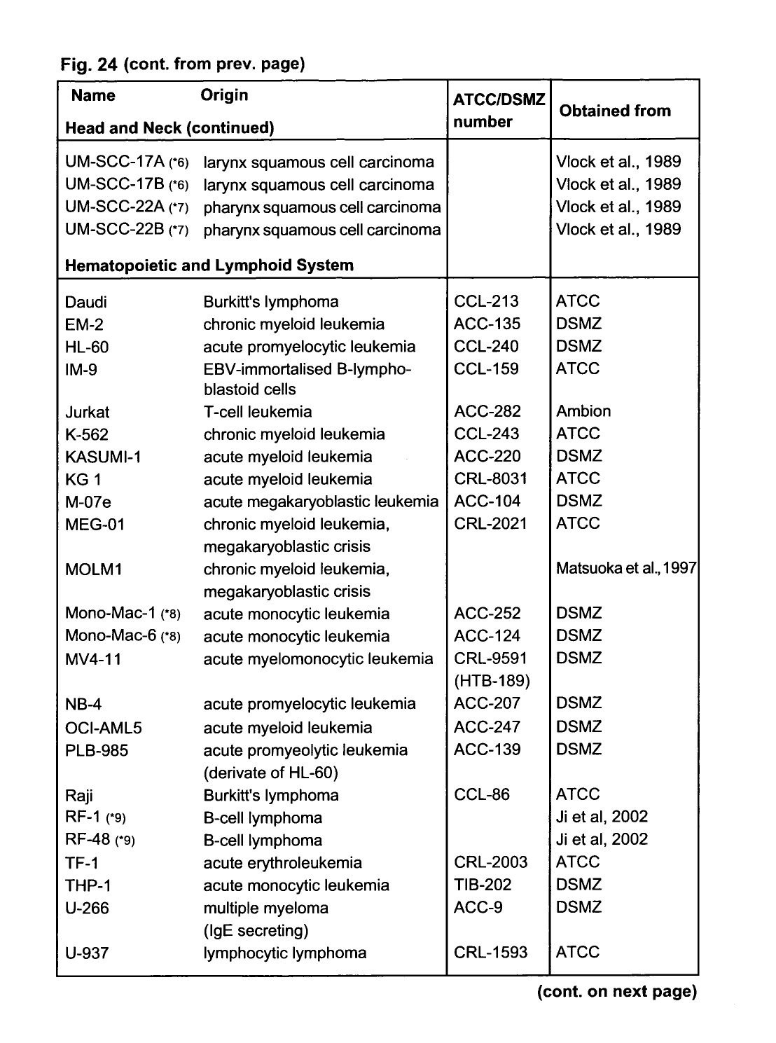

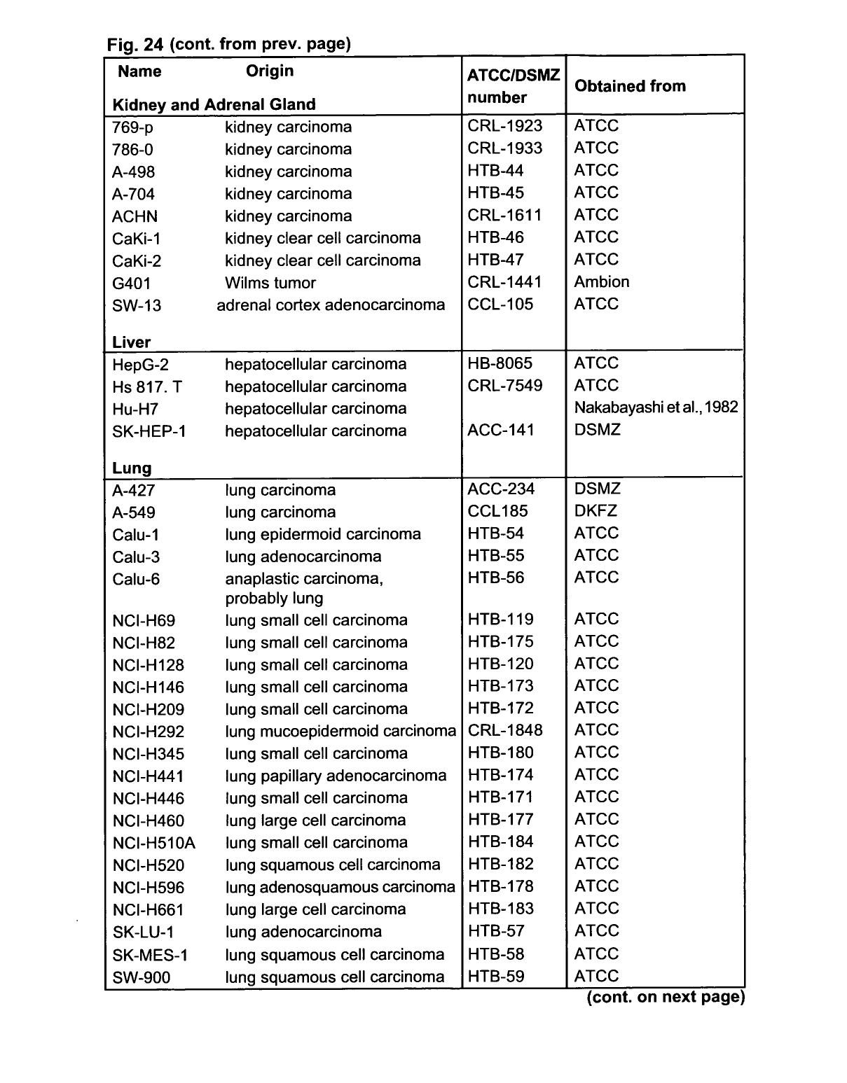

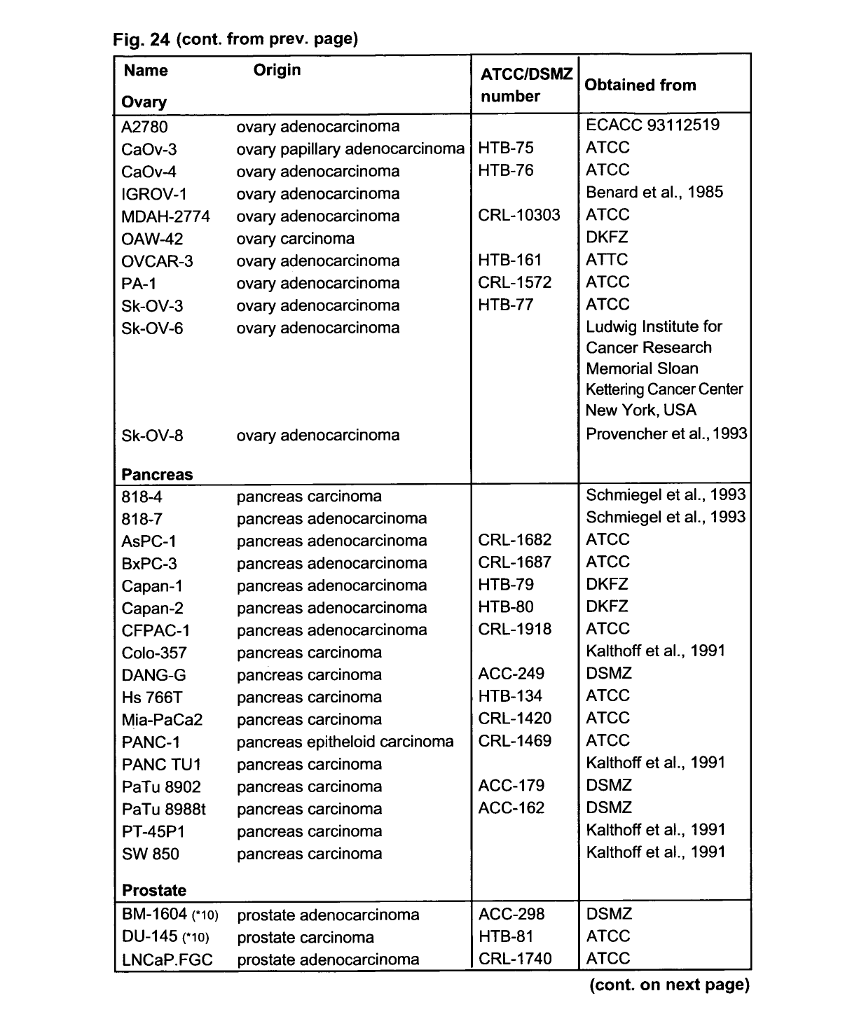

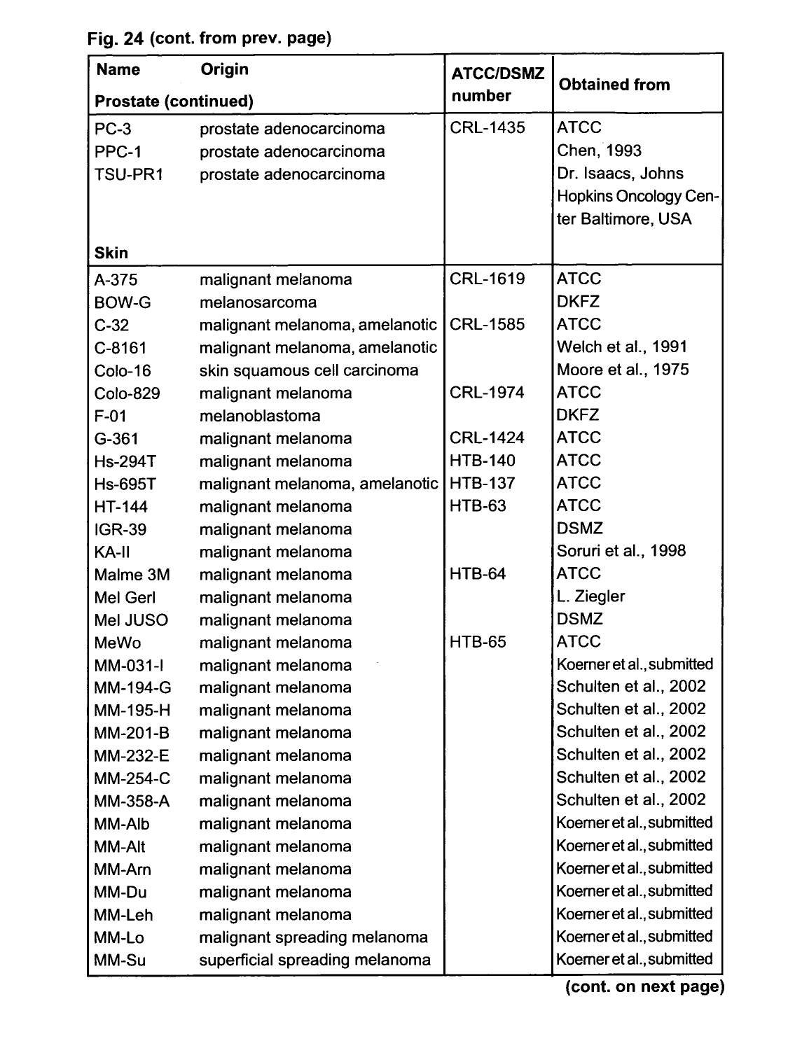

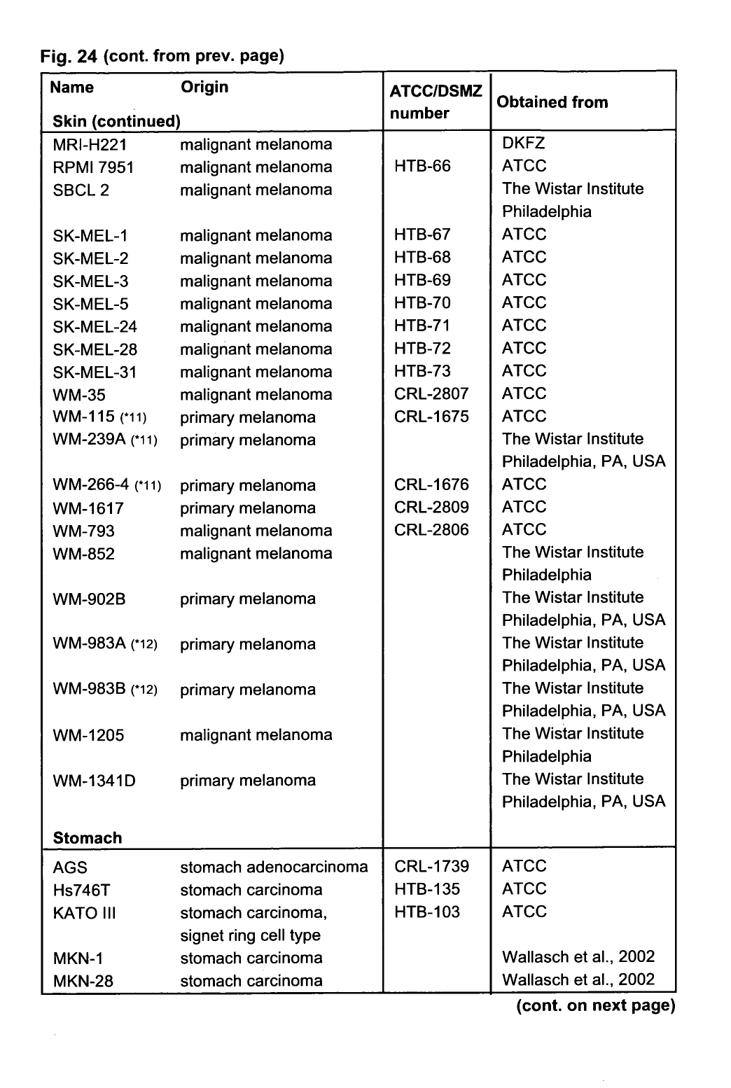

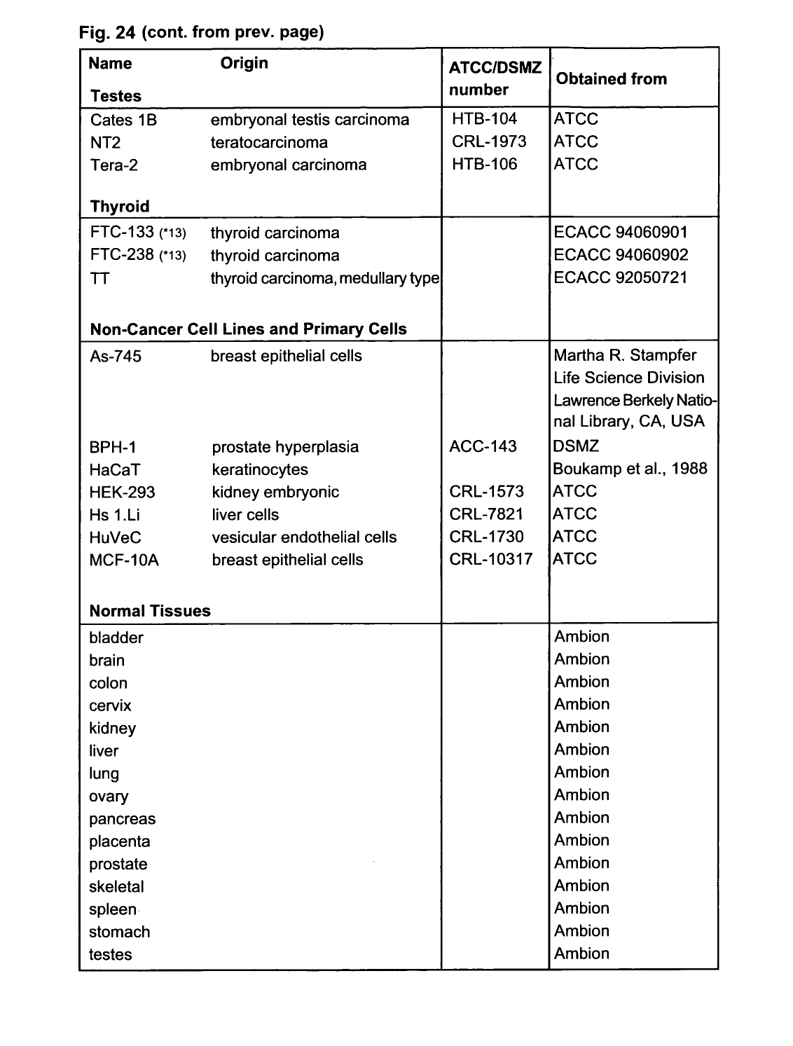

Sequence traces were assembled and analyzed to identify potential genomic alterations using the Mutation Surveyor software package (SoftGenetics, State College, Pa.). The tyrosine kinase gene sequences were aligned with the NCBI reference sequence (FIG. 24) and identified alterations were compared with the literature or public databases such as the NCBI SNP database (http://www.ncbi.nlm.nih.gov), the Ensemble Genome Browser (http://www.ensembl.org), the UniProtKB/Swiss-Prot database (http://ca.expasy.org) the SNP500Cancer database, and KinMutBase (http://bioinf.uta.fi/KinMutBase/main_frame.html).

Despite the lack of normal tissue counterparts for the established tumor cell lines, it was attempted to define the identified PTK transcript variations as somatic or germline sequence differences. Those sequence differences ere defined as germline polymorphisms that were either detected in our 22 controls or were previously reported as hereditary variants in the databases named above or the literature. If genetic alterations occurred neither in the 16 normal tissues nor in one of the cited databases, it was defined as a somatic mutation. Besides zygosity, cell line-specific variant profiles thus indicate germline or somatic origin of the individual TKT sequence variations. Representative examples are displayed for the skin-derived tumor cell lines A-375, BOW-G, C-32, C8161, and Colo-16 (FIG. 1B). The characterization of all cell lines can be found in FIG. 30.

### Gene Identification

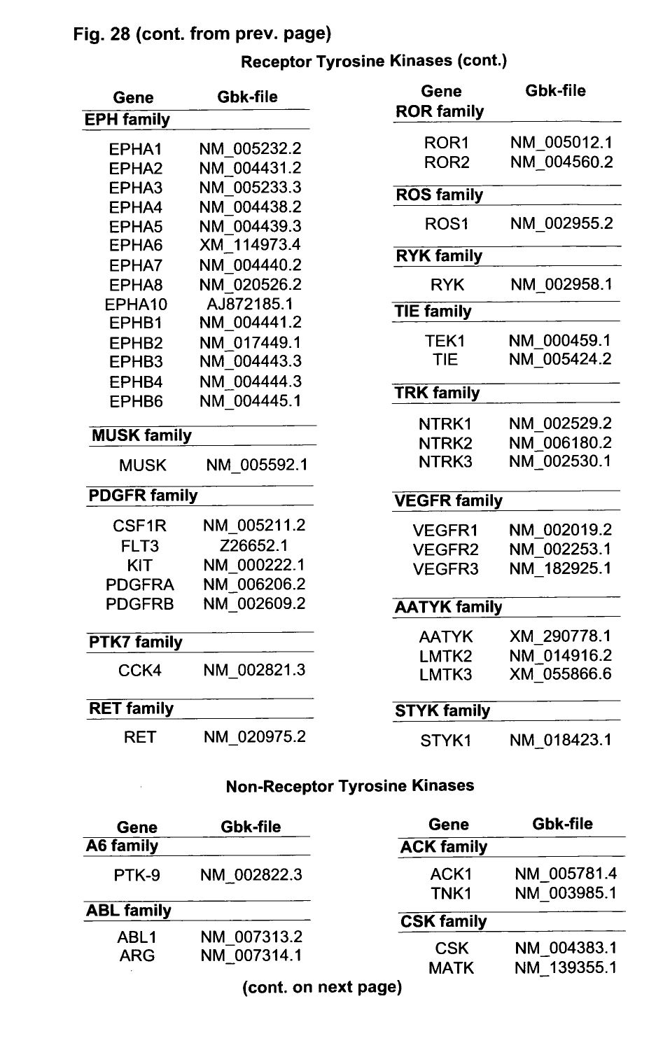

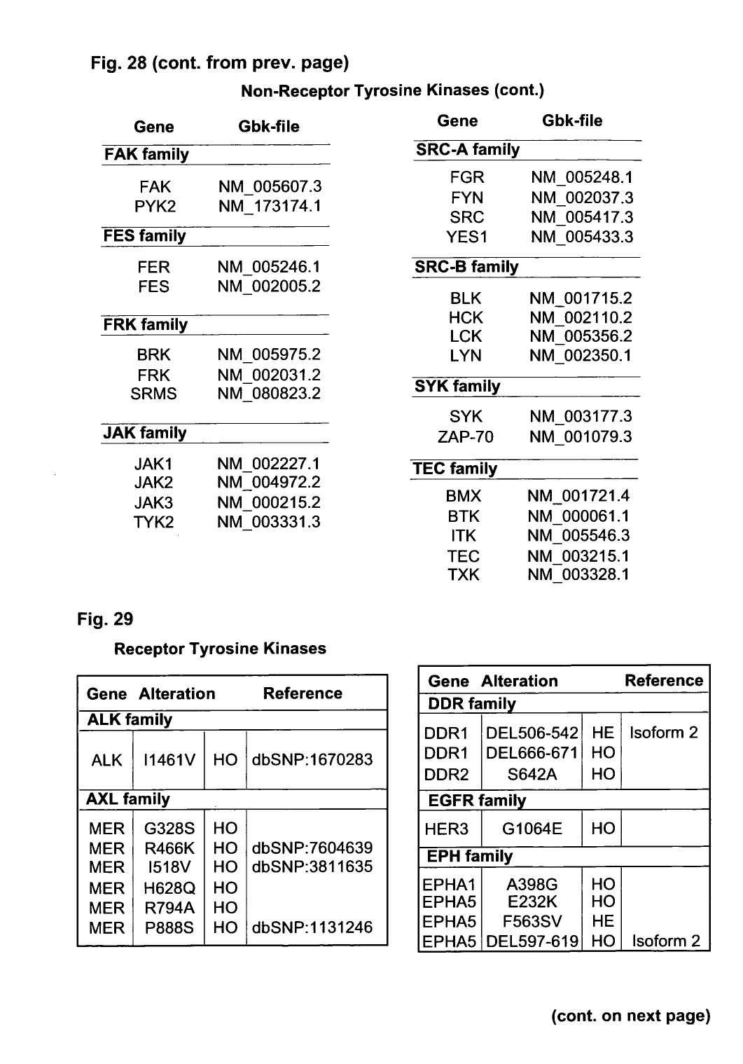

The coding sequences of the analyzed RTK and TK genes were retrieved from NCBI (www.ncbi.gov). NCBI accession numbers are provided in FIG. 28.

### Results

In order to comprehensively characterize widely-used tumor cell lines with regard to non-silent alterations in all expressed tyrosine kinase genes, the inventors evaluated the sequence of the entire tyrosine kinase transcriptome of 254 established cancer cell lines (FIG. 28). These cell lines were derived from 19 different tissue origins (FIG. 1A), controls included 7 non-tumorigenic cell lines and 15 tissues from different organs of healthy individuals.

### Identification of 422 Non Synonymous Genetic Alterations in the TKT of 258 Cancer Cell Lines

Based on the abovee data, the absolute number and distribution of TKT-linked somatic mutations and germline polymorphisms was determined within the entire tumor cell line panel. In total, 72.08 Mb of cDNA sequence encoding the entire protein tyrosine kinase gene family of 59 receptor tyrosine kinases and 32 cytoplasmic protein tyrosine kinases were analyzed. 39.85 Mb of reverse transcribed mRNA were amplified that, apart from IRR, MUSK, FGR and SRMS for which no PCR product from any of the cDNA samples was obtained, represent the entire protein tyrosine kinome expressed within the 280 samples examined. With this analysis numerous silent DNA sequence differences (not presented and further analyzed) and 389 non-synonymous genetic alterations were identified that were amplifiable and represent the detectable TKT of all samples. The majority of these—namely 359 sequence differences—were missense single base substitutions that caused amino acid changes, whereas only two somatic base replacements resulted in the generation of translational termination codons. Furthermore, 43 deletions and 18 insertions were detected. Remarkably, 65 sequence differences to the NCBI database occurred in all cDNA samples analyzed, strongly indicating that these variants actually represent the wild-type rather than genetic alterations (not shown) and were possibly caused by sequencing errors in the human genome database or the fact that the database entries represent individual sequence variants.

The basis for the discrimination between sporadically occurring somatic mutations and hereditary germline alterations or polymorphisms was formed by the 22 control samples of tissues from healthy individuals and non-tumorigenic cell lines, and by the extensive variant information obtained from public databases such as the NCBI SNP database (http://www.ncbi.nlm.nih.gov), Ensemble Genome Browser (http://www.ensembl.org), the UniProtKB/Swiss-Prot database (http://ca.expasy.org), or the SNP500Cancer database (http://snp500gov.nci.nih.gov). Those genetic alterations that were either identified in the control samples or have been reported as polymorphisms in one of the databases or the literature were considered as germline alterations.

For polymorphisms, a Gaussian-like distribution was observed, with an average of 12.3 sequence variations per cancer cell line. In contrast, somatic mutations were unevenly distributed (FIG. 1C). No somatic alterations were detected in the TKT of 119 cancer cell lines, consistent with kinome mutations entirely absent in subsets of recently screened breast cancer, lung carcinoid, and testicular germ-cell tumor samples (Stephens et al., Nat Genet. 37:590-592 (2005); Davies et al., Cancer Res 65:7591-7595 (2005); Bignell et al., Genes Chromosomes Cancer 2006; 45:42-6). In contrast, high frequencies of 9 to 14 somatic mutations in the transcribed tyrosine kinomes of LNCaP, Jurkat, MeWo, MKN1, HCT-15 and DLD-1 might reflect a mutator phenotype (Stephens et al., 2005, supra). They are in agreement with sequence data of 24 cancer genes in the NCI-60 cell line panel that also showed HCT-15 to be one of the most frequently mutated tumor cell lines (Ikediobi et al., Mol Cancer Ther 5:2606-12 (2006)). With intermediate mutation rates for the other tumor cell lines, our data indicate an accumulation of somatic mutations in PTK transcripts of various cancer cell lines which may contribute to the progression characteristics of certain cancers.

As alternative to the allocation to cell line-specific variant profiles, these sequence differences were grouped by genes and PTK subfamilies. Each variation was thereby specified regarding the spectrum of affected cell lines as well as the zygosity status and the presumable somatic versus germline origin. These data are shown for FGFR4 (FIG. 2) that will be discussed below. The full information for all transcript variants and PTK genes can be obtained from FIG. 31-FIG. 33.

### Tykiva Database for Cancer Cell Line TKT Analysis

Additionally, all data on the identified PTK transcript variants are compiled in the database designated “Tykiva” (tyrosine kinome variant; http://tykiva.bii.a-star.edu.sg). Transcript variants can be specifically retrieved for each of the 254 tumor cell lines, 19 tissue origins/tumor types, or any of the 90 PTK genes. Somatic or germline origin is indicated, and other cell lines carrying the same variant are referred to. In graphical gene representations, the localization of all detected variants is displayed in the context of the reference amino acid sequence as well as predicted protein domain structures according to Swiss-Prot data. Optionally projectable to the major known isoforms, these illustrations cross-reference our data to variant information from the NCBI SNP database10, the Ensemble Genome Browser, the Swiss-Prot and the GenBank databases, the KinMutBase, the IDbases, and the literature. By that, they include the current knowledge of non-silent genetic variations in PTK genes.

The expressed PTK variants may define cell line-specific signaling characteristics and cancer-related cell properties. In the following sections, tissue distribution and localization of each polymorphism and somatic mutation within the respective protein sequence are therefore addressed. Based on these data and the current literature, potential functional and/or clinical relevance for some of the identified genetic variants are discussed.

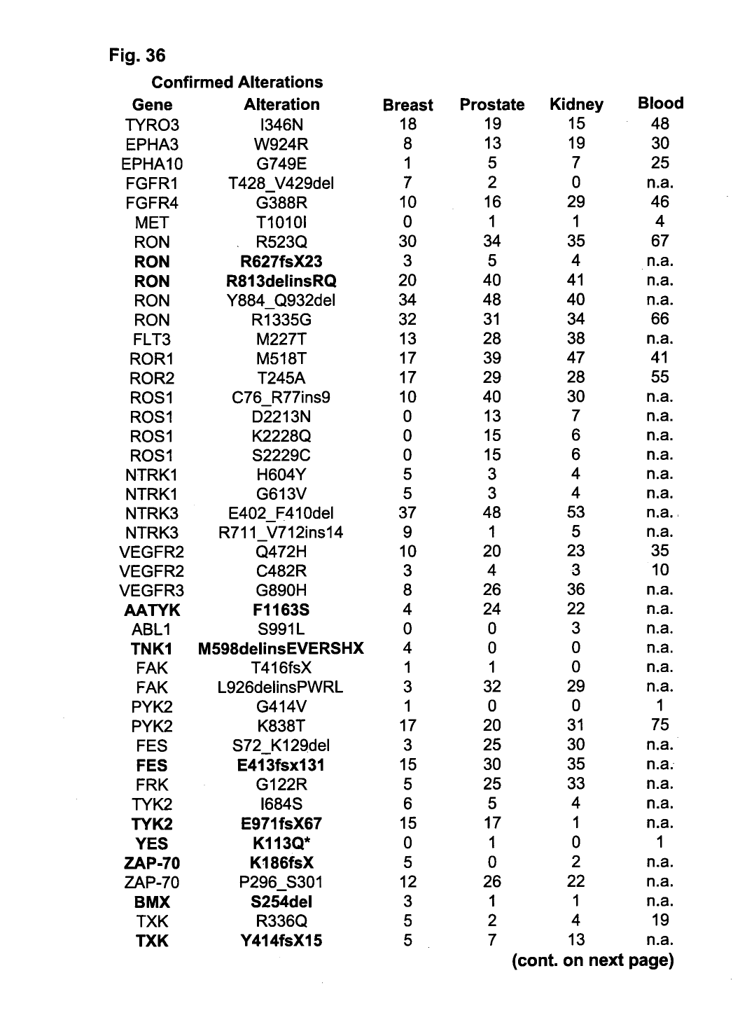

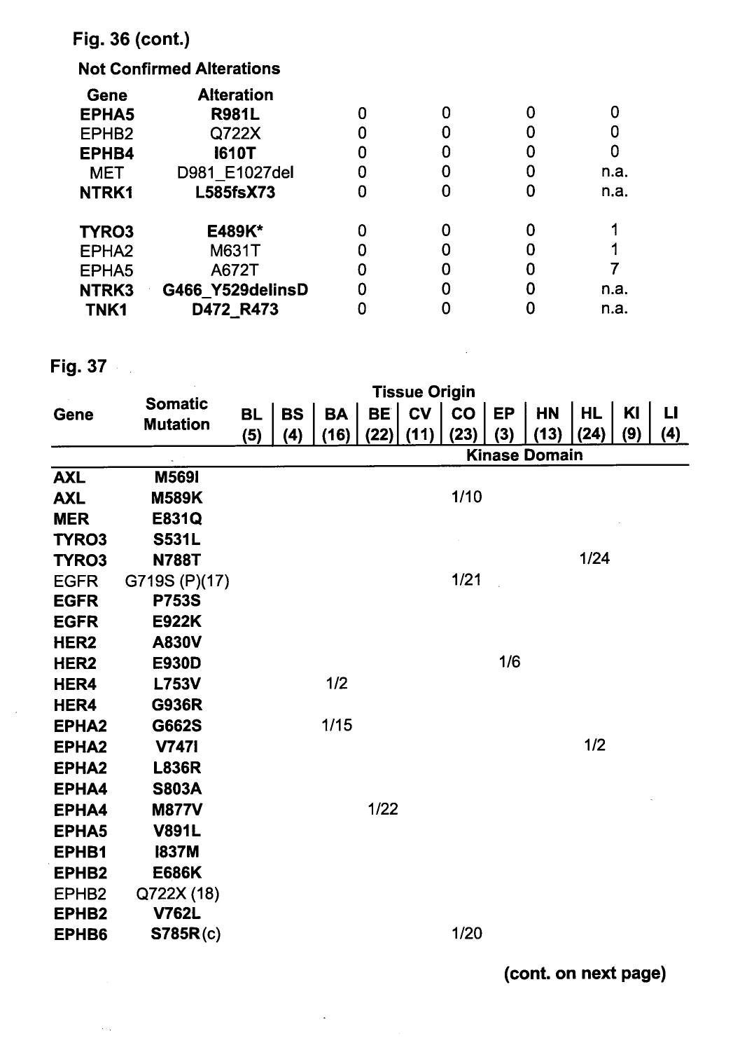

The genetic alterations analyzed in primary tumor samples are shown in FIG. 36. All non-conservative genetic alteration that were found at least twice in the panel of 276 cell lines and control samples with at least one cell line being derived from breast-, kidney- or prostate cancer were analyzed for occurrence in cDNA obtained from 55 primary breast carcinomas, 55 prostate cancer specimens, 55 kidney cancer specimens, 50 healthy kidney tissues and in genomic DNA derived from blood of 90 non-cancer donors. The number of carriers per sample group is provided for each alteration. Genetic alterations considered somatic as a result of the cell line screen are indicated with asterisks.

### Characterization of 155 PTK Gene Sequence Polymorphisms

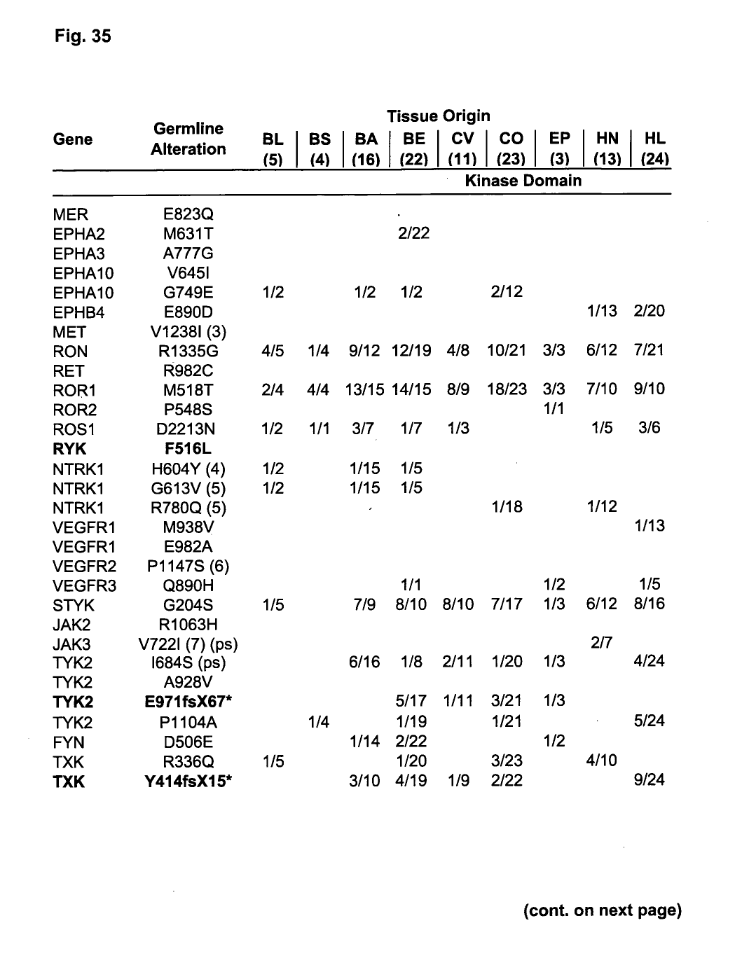

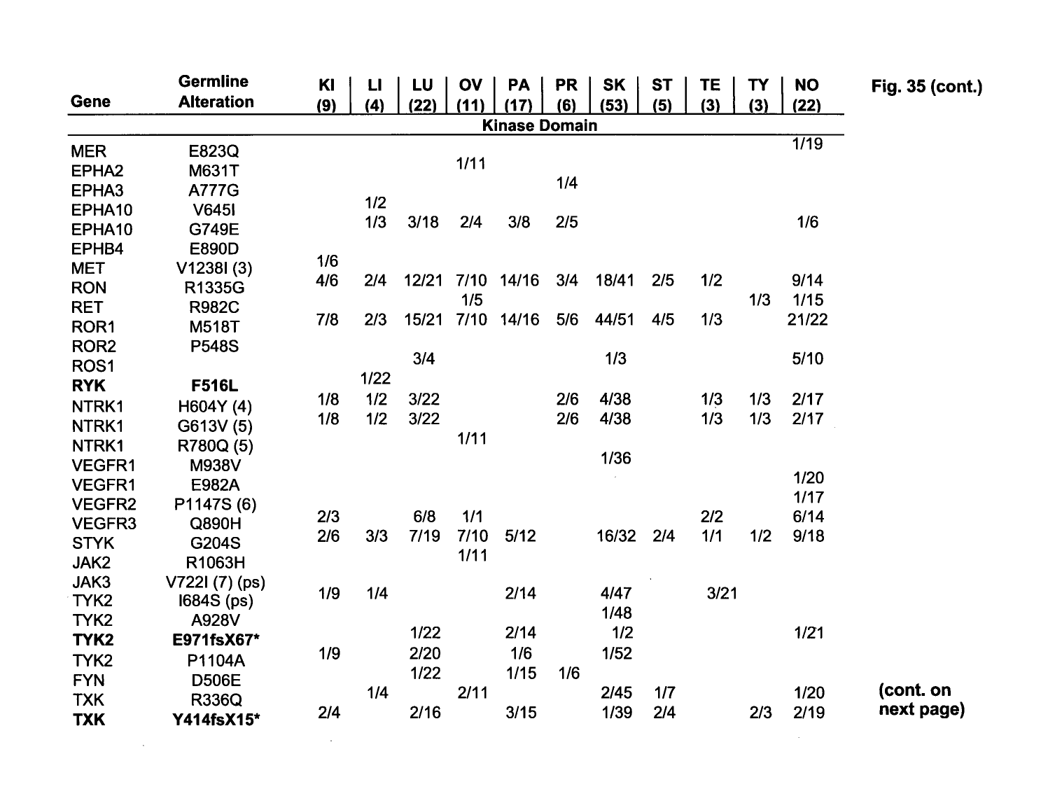

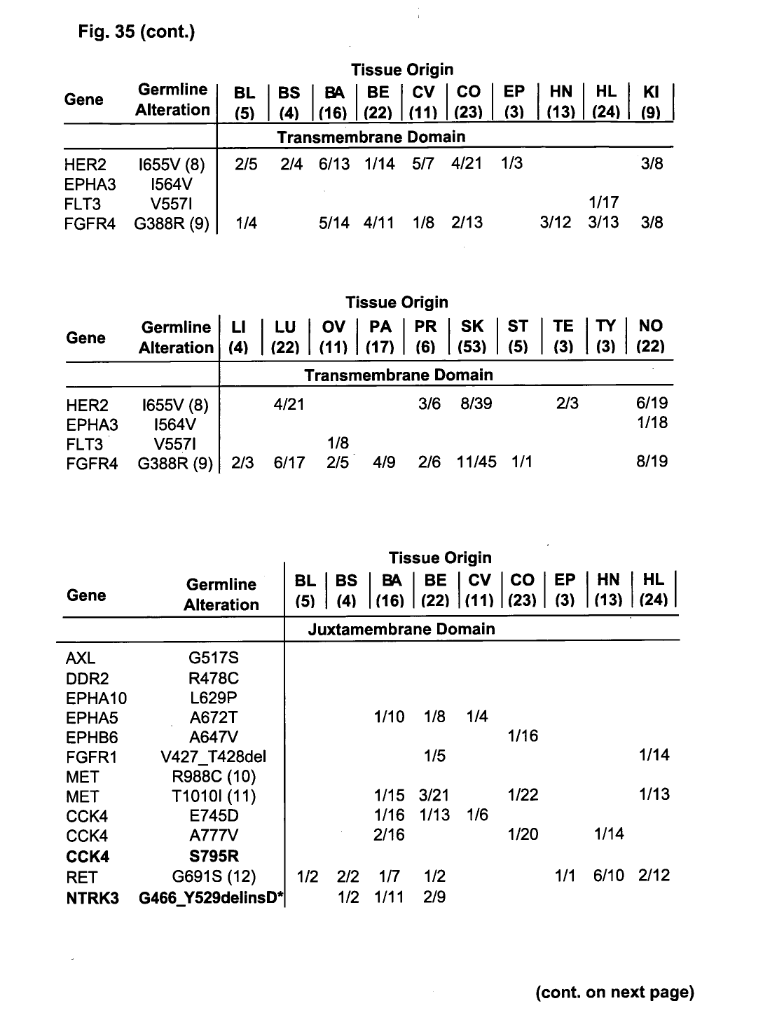

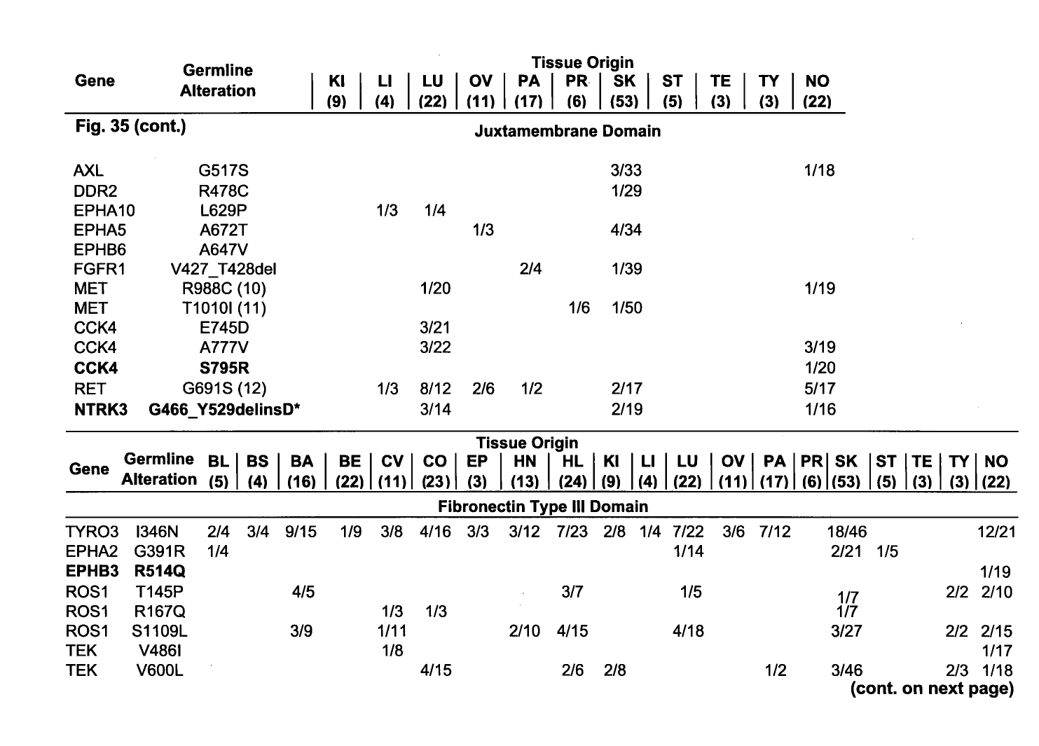

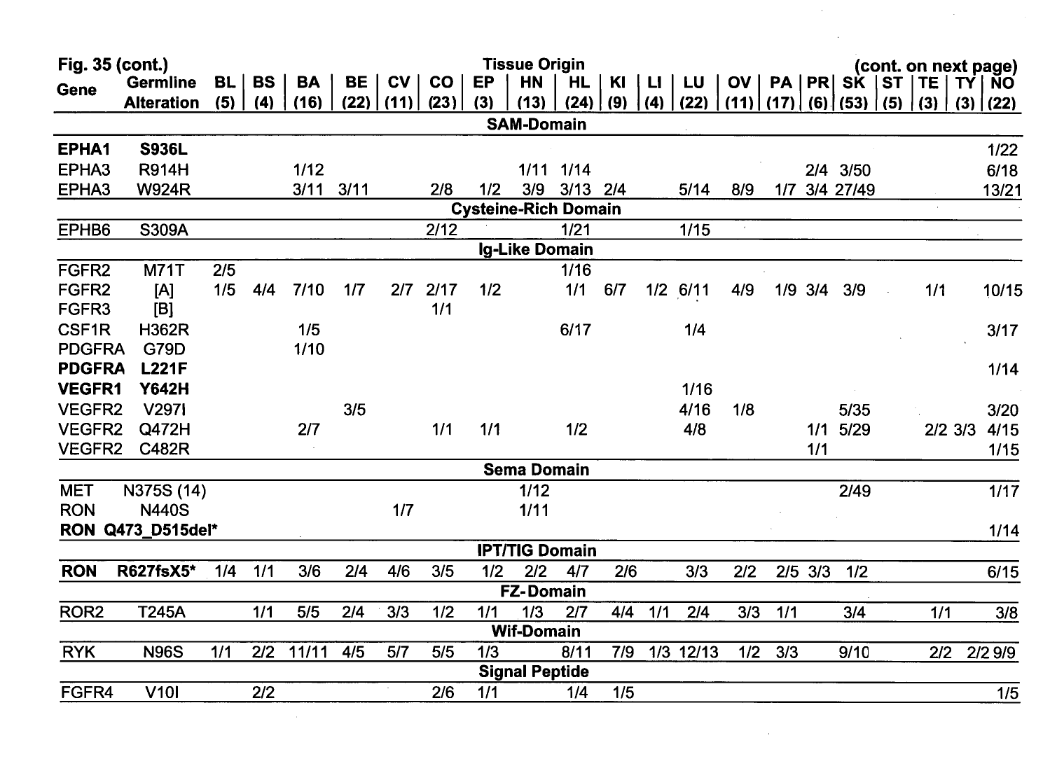

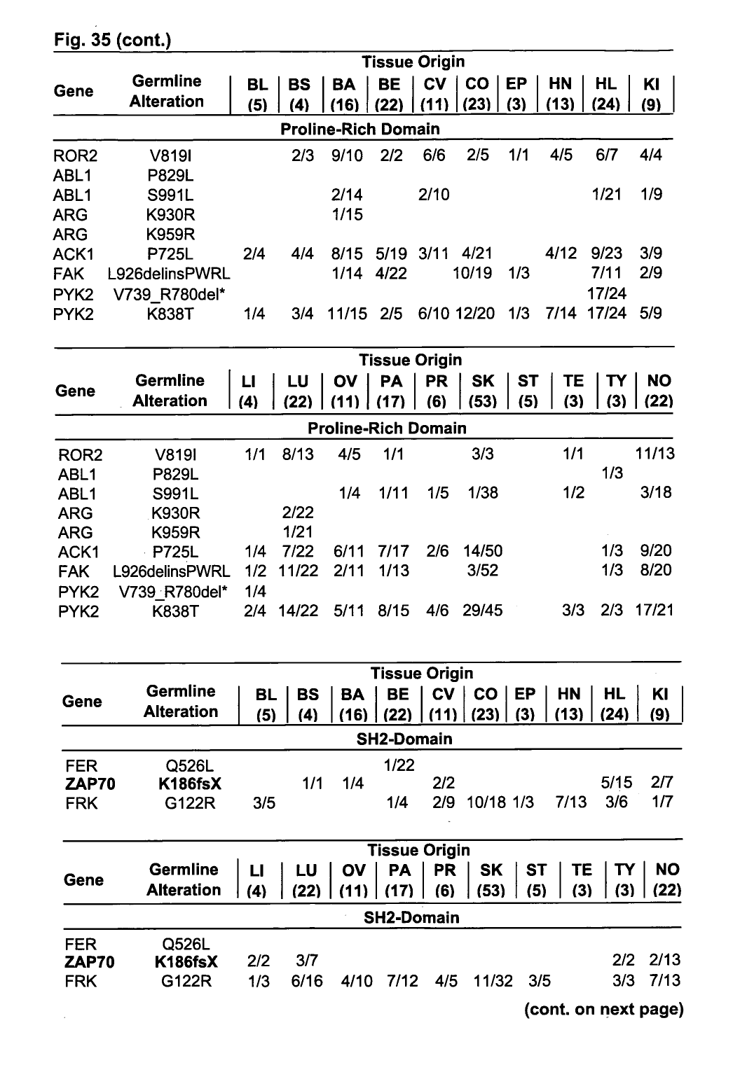

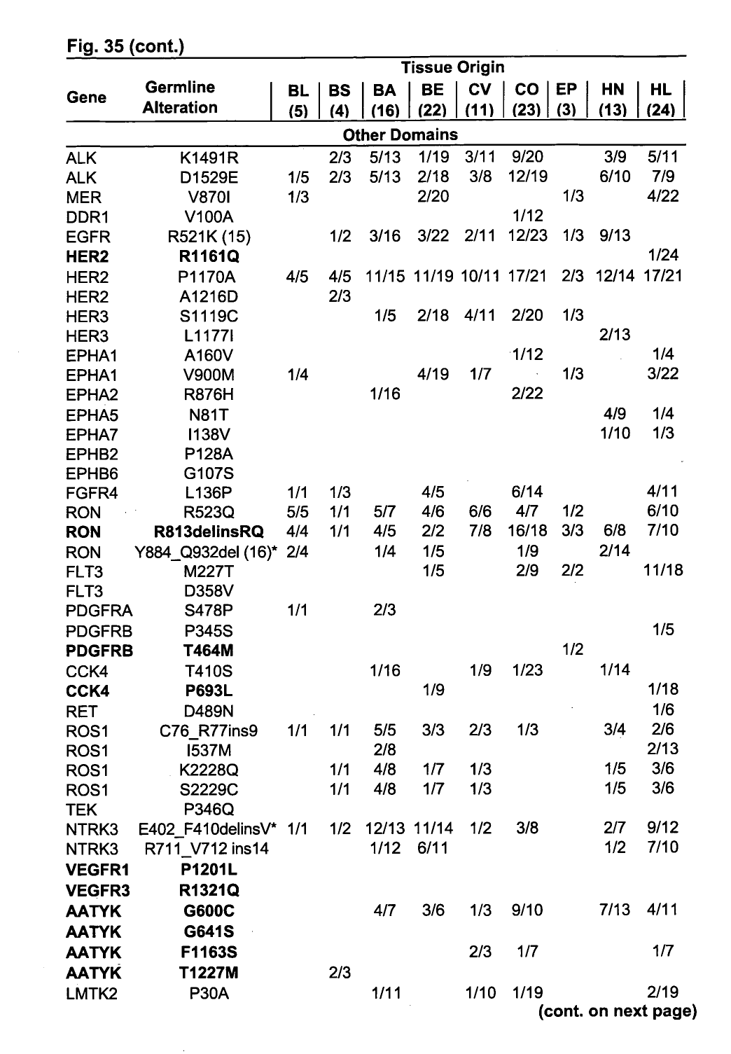

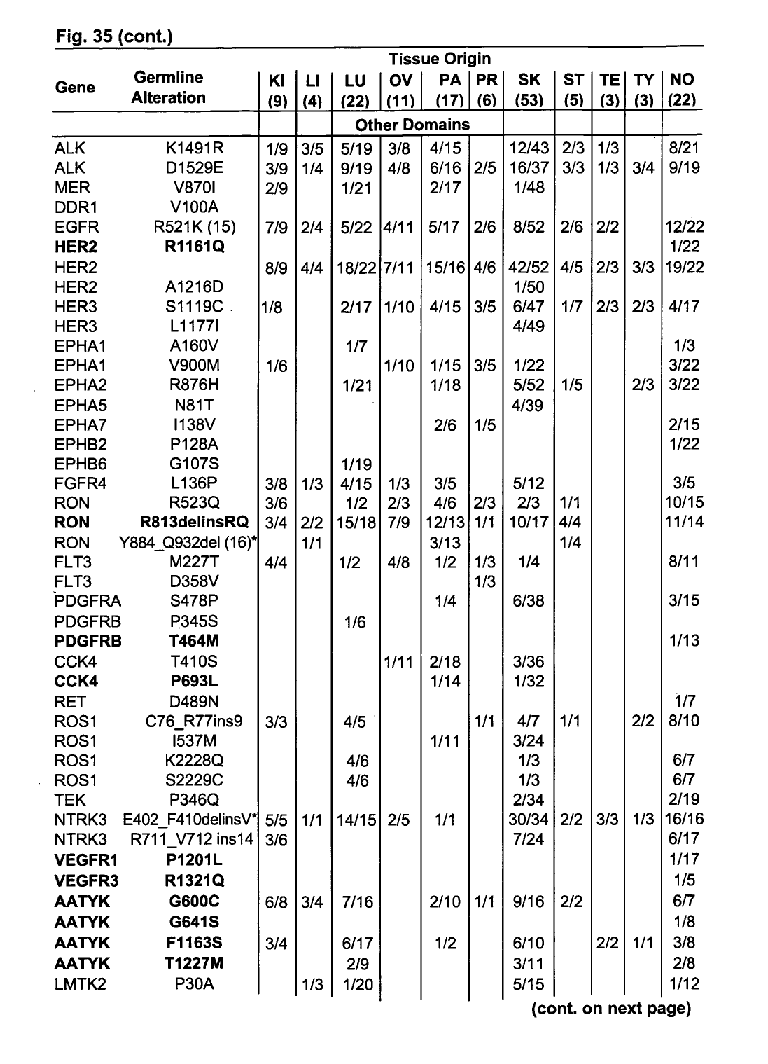

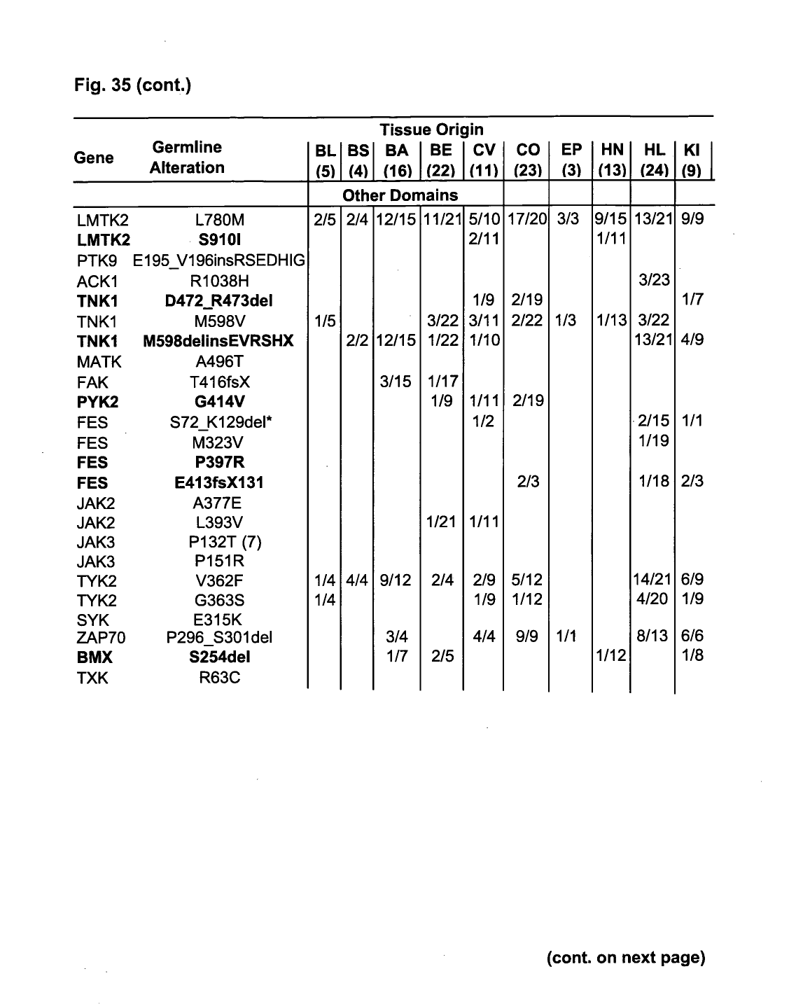

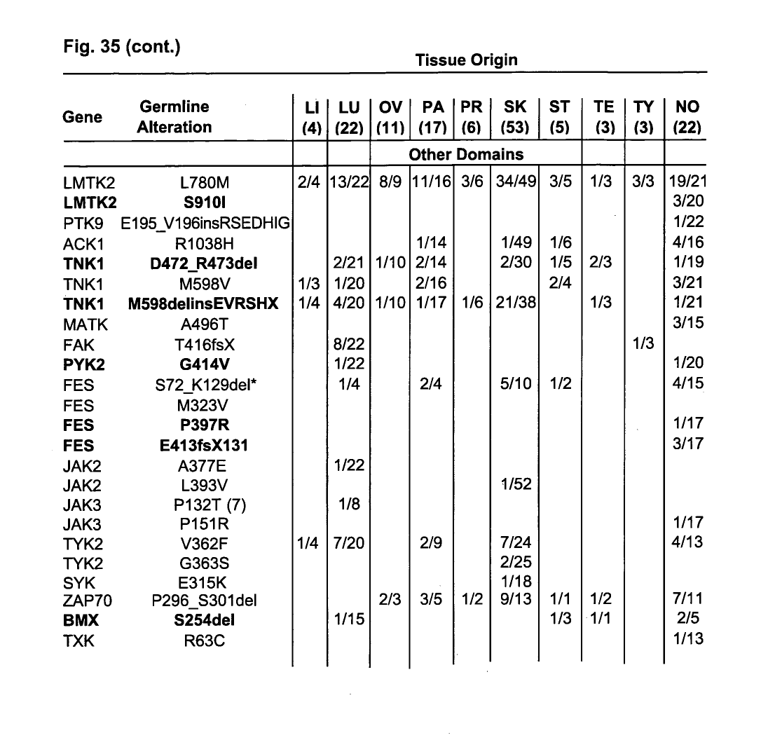

According to the above definition of somatic and germline sequence variants, 155 of the 389 identified alterations were classified as sequence polymorphisms. They include 131 SNPs, 16 germline deletions, and 8 insertions. Their overall frequencies and localization in distinct protein domains are summarized in FIGS. 3A and 3B. Moreover, the occurrence frequency of each polymorphism in individual tumor types or control samples was determined. Occurrence frequency was thereby defined as the fraction of carriers of a given sequence variant and the number of cell lines with the same tissue origin that express the corresponding gene regardless of its genotype (paired numbers in FIG. 3C and FIG. 35). It therefore reflects the expression aspect of respective genes and alterations, as addressed by our cDNA analysis.

Of the 131 missense substitutions, 100 had been reported previously. However, only 12 of them, as well as 2 deletions, have been connected with cancer so far (FIG. 35). Noteworthy, 5 of 8 novel deletions involve entire exons (FIG. 35) and most likely represent splice variants. Such variants could preferentially be detected because of the use of cDNA as sequencing target. Moreover, other transcription-related mechanisms such as epigenetic gene silencing or mRNA stability are also reflected by cDNA, and genetic alterations identified therein are thus likely to be expressed within the cell. However, a disadvantage of our approach is that it does not detect fusion kinase gene- or amplified kinase transcripts.

In order to verify the in vivo-relevance of the sequence variations detected in tumor cell lines, cDNA from 165 primary breast, kidney and prostate cancer specimens was analyzed as well as blood DNA from 90 healthy individuals for the occurrence of a representative subset of our identified genetic alterations. This subset was defined as all non-conservative sequence changes that were found at least twice in our panel of cell lines and control samples with at least one cell line originating from breast, kidney, or prostate cancer. All but 2 of the 46 polymorphisms that fulfilled these criteria could be verified in patient sample cDNAs or blood DNA (FIG. 36), hence confirming the in vivo-relevance of sequence variations in tumor cell lines.

Noteworthy, some novel polymorphisms have been reported as somatic mutations in the literature before. VEGFR2 P1147S, a non-conservative substitution within the catalytic domain of VEGFR2/KDR, for example, has been described as somatic mutation in hemangioma specimens (Walter et al. (2002) Genes Chromosomes Cancer 33: 295-303). This variant was identified in skeletal muscle tissue from a healthy control individual, clearly demonstrating its germline origin and supporting the assumption that various alterations reported earlier as somatic mutations might actually represent germline polymorphisms.

### Cancer Relevance of Identified Polymorphisms

More and more evidence has accumulated over the past years that indicates that genetic polymorphisms can significantly influence clinical parameters of human cancers. In order to provide first information on possible structural or functional consequences of individual polymorphisms the location of all identified polymorphisms within the respective protein sequence is presented herein. In FIG. 37, germline alterations in regions coding for the different domains, including kinase-, the transmembrane- and the juxtamembrane domains, are displayed. Furthermore, the tissue origins a given polymorphism has been found in are indicated (BL: bladder; BS: bone and soft tissue; BA: brain; BE: breast; CV: cervix and vulva; CO: colon; EP: endometrium and placenta; HN: head and neck; HL: hematopoietic and lymphoid system; KI: kidney; LI: liver; LU: lung; OV: ovary; PA: pancreas; PR: prostate; SK: skin; ST: stomach; TE: testes; TY: thyroid, NO: normal control samples).

Two polymorphisms in the cytoplasmic tyrosine kinase TYK2 and TXK transcripts, TYK2 E971fsX67 and TXK Y414fsX15, are remarkable because of their frequency and localization. TYK2 E971fsX67 and TXK Y414fsX15 were found in different cDNAs, and thus represent relatively frequent germline alterations. Both variations affect the tyrosine kinase domain, and in both cases, the deletion represents the loss of an entire exon associated with a frame shift and the generation of a premature translation termination site. For TYK2 E971FsX67, exon 19 beginning with the N-terminal Glu⁹⁷¹ was deleted, and in conjunction with the frame shift (fs) leading to a STOP-codon after another 67 amino acid residues, this resulted in the loss of the major part of the catalytic domain including the catalytic and the activation loop. The deletion of exon 13 in TXK (TXK Y414fsX15) also results in a truncated variant lacking the functional kinase activation loop and the subsequent C-terminal region, and thus is also very likely to be associated with disruption of catalytic activity.

The polymorphic in-frame deletion NTRK3 G466_Y529delinsD was found to affect the juxtamembrane membrane of this RTK in two brain, breast and skin tumor cell lines each as well as in three lung cancer and one control cell line. With exception of 12 amino acid residues that are most proximal to the transmembrane domain and 8 residues preceding the kinase domain, this deletion of exon 13 results in the loss of the entire juxtamembrane region. All deletions were detected in one allele only.

The single nucleotide polymorphism ACK1 P725L is among the novel germline variants the one with the highest frequency. It was detected in 80 different cancer cell line cDNA samples. Interestingly, Pro⁷²⁵ is one of the proline residues that define the affected region of ACK1 as a proline-rich domain. Similarly, in the cytoplasmic tyrosine kinase FAK the proline-rich domain was found to be affected by L926delinsPWRL. In contrast to ACK1 where Pro⁷²⁵ was replaced by an isoleucine, the substitution of FAK Lys⁹²⁶ by the polypeptide PWRL led to the insertion of an additional proline. In both cases the affinity and/or specificity for interacting binding proteins might be modulated.

In addition to the localization within the protein, strikingly different frequencies in particular tumor types might provide another hint for the potential cancer relevance of a given sequence alteration. The present inventors therefore determined the number of cDNA samples carrying a given germline variation of the reference sequence and the fraction of cell lines that express the corresponding gene (indicated by paired numbers in FIG. 37). As can be seen from FIG. 37 the representation frequency of several polymorphisms varies significantly in tumor cell lines of different tissue origins indicating the possibility of respective differential functional relevance.

Some of the identified polymorphisms have previously been associated with non-proliferative diseases. Respective functional modulations may, because of the pleitropic effects of many PTKs, also be relevant for cancer. This is exemplified by the V722I transversion in the pseudokinase domain of JAK3 that the inventors identified as a rare heterozygous polymorphism in the head and neck cancer cell lines SCC-10A and SCC-10B (FIG. 3C). First reported in patients with autosomal recessive T-B+ SCID syndrome (Schumacher, R. F., et al., Hum Genet. 106:73-79 (2000)), its recent detection in an acute megakaryoblastic leukemia (AMKL) patient and the capacity to transform Ba/F3 cells (Walters et al., Cancer Cell 10:65-75 (2006)) support a potential role in cancer. Another example is NTRK1 R780Q which the inventors found in the colon-, ovarian-, and head and neck cancer cell lines Caco2, SK-OV-8 and SCC-9 (FIG. 3C), respectively. This SNP affects the same arginine residue whose replacement with proline was shown to be associated with “congenital insensitivity to pain with Anhidrosis (CIPA)” and abrogation of catalytic tyrosine kinase activity in vitro (Greco e al., Am J Hum Genet. 64:1207-10 (1999)). Assuming a similar loss-of-function for the NTRK1 Q780 isotype, this variant may exert anti-apoptotic and hence pro-oncogenic effects, as expression of NTRK1 wild type was associated with induction of apoptosis and a favorable prognosis of neuroblastoma patients (Lavoie et al., J Biol Chem 280:29199-207 (2005)).

Cancer relevance was also established for MET T1010I which represents a biomarker for MET inhibitor efficacy (Jagadeeswaran et al. Cancer Res 66:352-61 (2006)) and was originally reported as a somatic gain-of-function mutation in small- and non-small cell lung cancers (SCLC, NSCLC; Ma et al., Cancer Res 63:6272-6281 (2003)) and malignant pleural mesotheliomas (MPM; Jagadeeswaran et al., 2006, supra). Its detection in 4 of our 90 blood control DNAs (FIG. 36), however, confirmed previous hints for potential germline occurrence (Tengs et al., Cancer Lett 239:227-233 (2006)). Moreover, the identification of MET T1010I in the prostate carcinoma cell line TSU-PR1 and a primary prostate tumor as well as in the brain-, breast-, colon-, hematopoietic- and skin cancer cell lines IHR-32, DAL, LS-123, U-266, and Colo-829, respectively (FIG. 3C and FIG. 36), suggests enhanced MET signaling in these tumor cell lines and expands the currently reported spectrum of affected tumor types.

### Polymorphism Frequencies in Cancer Cells Versus Normal Tissues

Differential occurrence rates of sequence polymorphisms in particular cancer types and/or normal tissues may indicate tumor suppressive or promoting effects. In order to address the potential relevance of all polymorphism for certain tumor types, their occurrence frequencies in tissue types and control samples was compared (FIG. 3C and FIG. 35). Only some examples are displayed in FIG. 4. For EGFR R521K, a relative over-representation of the EGFR K521 allele in cDNAs of normal control samples (55%), colon (52%), and head and neck (69%) tumor cell lines was detected (FIG. 4A) This indicates a possible tumor suppressive activity of the EGFR K521 isotype which apparently is not relevant to colon cancer and head and neck cancer. An attenuated growth response to EGFR ligands and reduced induction of the proto-oncogenes FOS, JUN, and MYC in EGFR K521, but not EGFR R521 expressing cells (Moriai et al., Proc Natl Acad Sci USA 91:10217-10221 (1994)), and an increased risk of local recurrence after chemoradiation treatment for rectal cancer patients with at least one EGFR R521 allele (Zhang et al., Clin Cancer Res 11:600-5 (2005)) support these conclusions. Similarly, a clearly differential occurrence of TYK2 F362 allele carriers was observed in brain—(75%) and hematopoietic/lymphoid system—(67%) derived tumor cell lines compared to control tissues (31%) or other tumor types (FIG. 4B. The novel polymorphism TNK M598delinsEVRSHX was found at low frequencies in control samples (5%) and cancer cells of several tissue origins, but occurred in 62% of blood-, 55% of skin- and, even more prominent, 80% of brain-derived tumor cell lines (FIG. 4C) In contrast to EGFR R521K, the under-representation of the TYK2 F362 allele and the TNK insertion in control samples indicates a tumor-promoting function with particular relevance for leukemia, melanoma, and glioma. It can be expected that, as for EGFR R521K (Zhang et al., 2005, supra) or FGFR4 G388R (Bange et al., Cancer Res 62:840-847 (2002)), the correlation with clinical parameters will assign therapeutic and/or predictive value to many of such unequally distributed alleles.

The domain localization and tissue distribution of identified polymorphisms is depicted in FIG. 35. The localization in distinct protein domains and the tissue distribution (BL: bladder; BS: bone and soft tissue; BA: brain; BE: breast; CV: cervix and vulva; CO: colon; EP: endometrium and placenta; FIN: head and neck; HL: hematopoietic and lymphoid system; KI: kidney; LI: liver; LU: lung; OV: ovary; PA: pancreas; PR: prostate; SK: skin; ST: stomach; TE: testes; TY: thyroid, NO: normal control samples) was determined for all polymorphisms. Paired numerals reflect the number of carriers of the indicated isotype as a subset of all cell lines within the given tissue origin that express the corresponding gene regardless of its genotype. Novel germline alterations are highlighted in bold type, polymorphisms located in the pseudokinase domain of JAK-family members are indicated as (ps), and skipping of entire exons is marked by asterisks. Parenthesized numbers refer to references given in the legend of FIG. 3 that associate indicated polymorphisms with cancer.

### Polymorphisms Affecting the Kinase Domain

The localization of genetic alterations within the respective protein sequence may be indicative of structural and/or functional consequences. The skipping of entire exons in the cytoplasmic kinases TYK2 and TXK, TYK2 E971fsX67 and TXK Y414fsX15, for instance, results in frame shifts and premature translation termination in the tyrosine kinase domains, and thus most likely is associated with catalytic inactivation. The truncated TYK2 variant that lacks 206 aa of the kinase domain including the catalytic site and the activation loop may also impose a dominant negative effect on cell signals. Interestingly, Stoiber et al. reported that TYK2-deficient mice developed B and T lymphoid leukemia with higher incidence and shortened latency as a result of decreased cytotoxic capacity of TYK2−/− NK and NKT cells and thus impaired tumor surveillance (Stoiber et al., J Clin Invest 114:1650-1658 (2004)). Since NK activity as part of the innate immune system mediates tumor rejection in general, the significance of TYK2 loss-of-function might not be restricted to hematopoietic malignancies, but may also be important for other cancer types. Consistent with this possibility, TYK2 E971fsX67 was detected in cancer cells derived from various tissues including breast, cervix/vulva, colon, endometrium, lung, and pancreas (FIG. 3C) as well as 33 clinical breast, prostate, and kidney cancer specimens. Its occurrence in the control cell line BPH-1 suggests potential germline origin. Hence, the TYK2 E971fsX67 splice variant may also represent a prognostic marker for cancer patients and support therapeutic decision making.

Overall, these examples point at the potential role of sequence polymorphisms as genetic parameters that may contribute to a patient-specific definition of disease predisposition, rate of progression, or responsiveness to therapeutic agents. In conjunction with simple detectability in blood samples, this renders polymorphisms to be highly valuable biomarkers for diagnostic patient characterization.

### Identification of 256 Somatic Mutations in Cancer Cell Lines

Of all sequence differences, 234 were undetectable in any of the control samples or public databases and were thus defined as somatic mutations. However, because of the lack of cell line-specific normal tissue controls, the possibility cannot be excluded that some actually represent rare germline polymorphisms. The somatic mutations are composed of 210 missense and 2 nonsense single nucleotide substitutions as well as 19 deletions and 3 insertions. While the majority (186) occurred once, 53 were found 2 to 5 times, and 3 in 6 to 10 tumor cell lines (FIG. 5A). Among the twice occurring somatic mutations, 20 were detected in cell lines originating from the same tumor donor (FIG. 24). They may be considered single mutations, thus adding up to a total of 206 non-recurring mutational events. As for the polymorphisms, all somatic TKT alterations are presented in the context of the respective protein domains and tumor types. Further, the ratio of affected and expression-positive cell lines are presented for each tissue origin (FIGS. 5B and 5C and FIG. 37).

The domain localization and tissue distribution of identified somatic mutations are summarized in FIG. 37. Somatic mutations are characterized with regard to their localization within the protein and the tissue origin (BL: bladder; BS: bone and soft tissue; BA: brain; BE: breast; CV: cervix and vulva; CO: colon; EP: endometrium and placenta; HN: head and neck; HL: hematopoietic and lymphoid system; KI: kidney; LI: liver; LU: lung; OV: ovary; PA: pancreas; PR: prostate; SK: skin; ST: stomach; TE: testes; TY: thyroid, NO: normal control samples) of affected cell lines. The first of the paired numerals provided for a given somatic mutation and particular tumor type indicates the number of mutated cell lines, the second numeral refers to the number of expression-positive cell lines. Somatic mutations affecting the activation loop (a), the catalytic loop (c), the P-loop (p) or the pseudokinase (ps) domain are indicated. Novel somatic mutations are highlighted in bold type, skipping of entire exons is indicated by asterisks. Numbers in parenthesis refer to references given in the legend of FIG. 3 (supra) that associate indicated mutations with cancer.

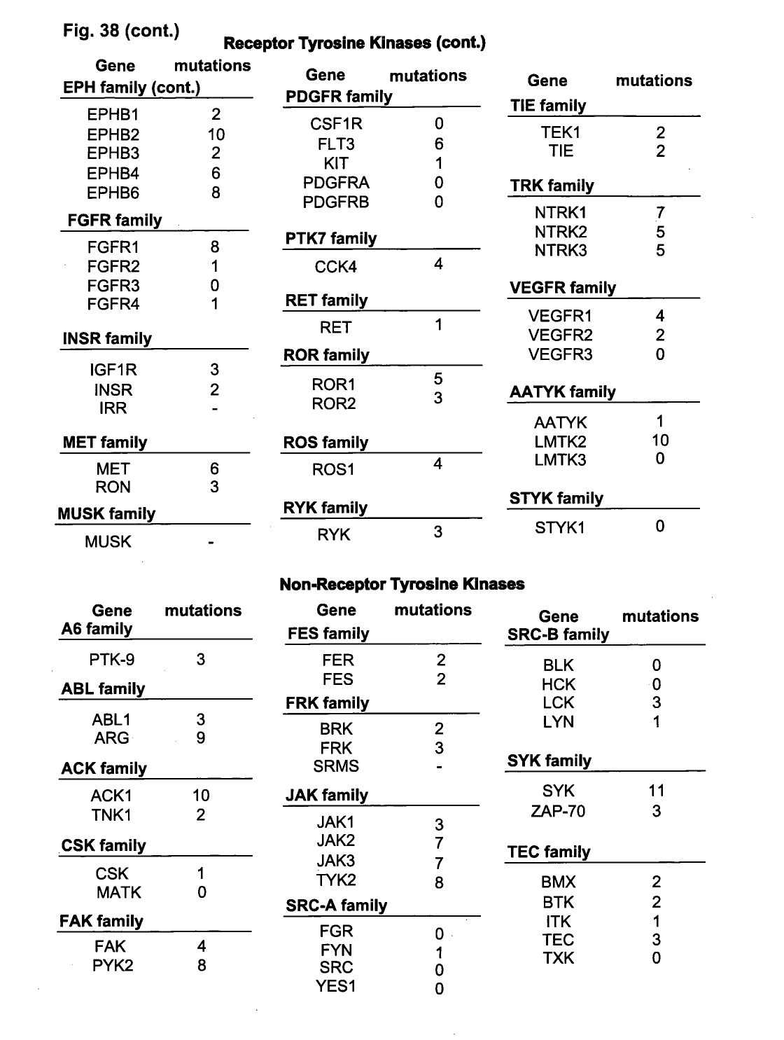

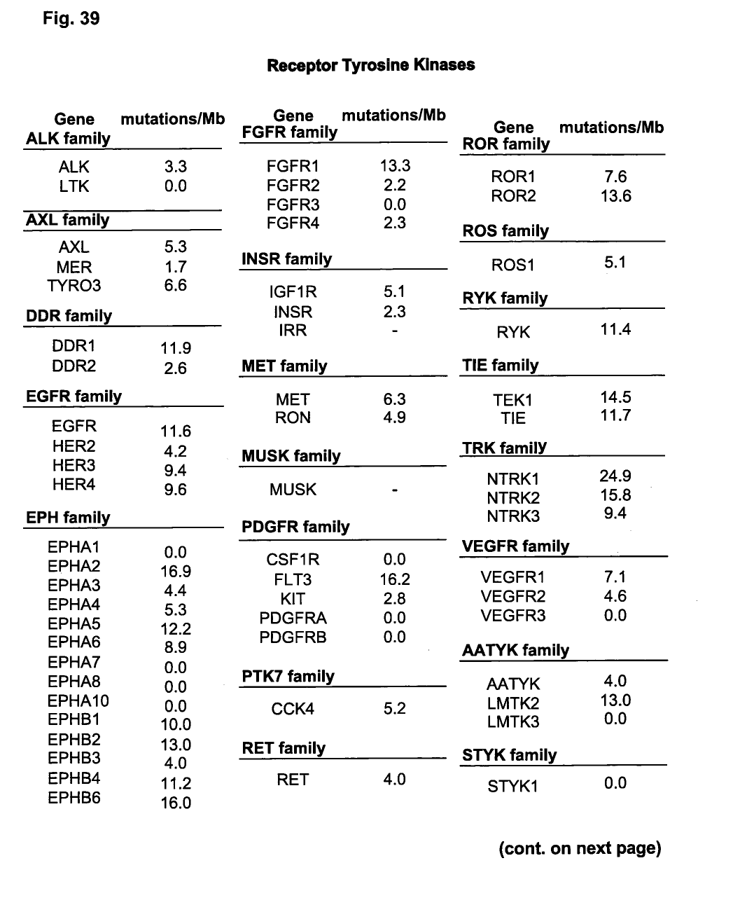

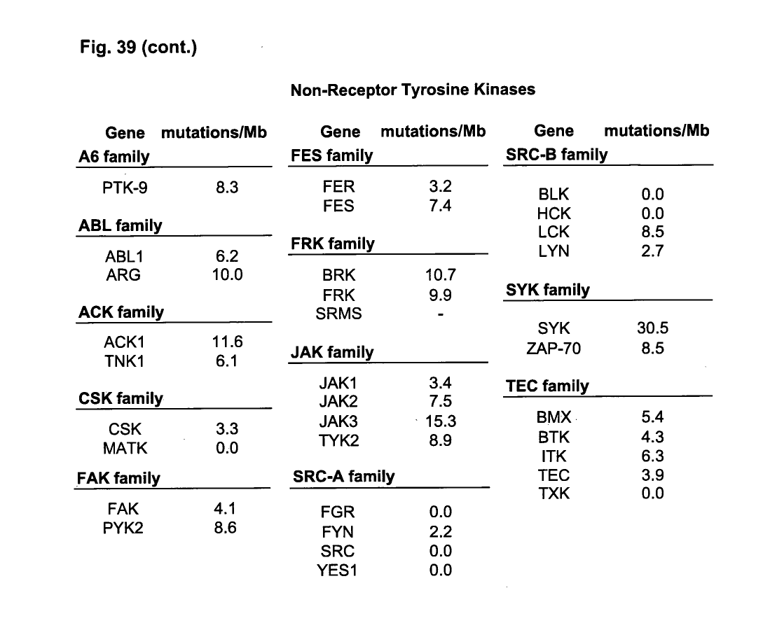

Consistent with SYK A353T to represent one of the 2 most prevalent mutations, the tumor suppressive tyrosine kinase SYK turned out to be the most frequently mutated kinase within our panel of 254 tumor cell lines. When absolute numbers of somatic mutations were compared, SYK scored highest with mutations detected in 11 non-related tumor cell lines (FIG. 38). After normalization with respect to the PTK transcription status, SYK showed the highest mutation rate of 30.3 sporadic alterations per 1 MB expressed coding sequence, followed by NTRK1, EPHA2, and FLT3 (FIG. 39). The domain organization of SYK and the known and novel genetic alterations are illustrated in FIG. 6A.

Somatic Mutations with Possible Oncogenic Potential

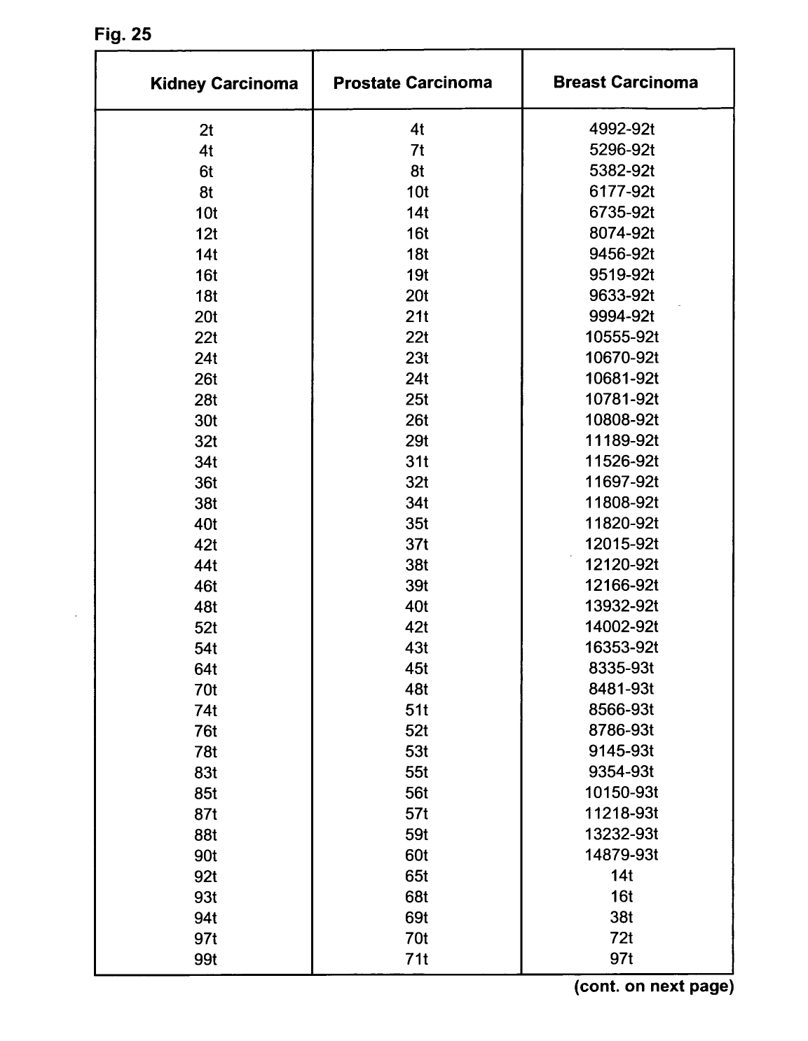

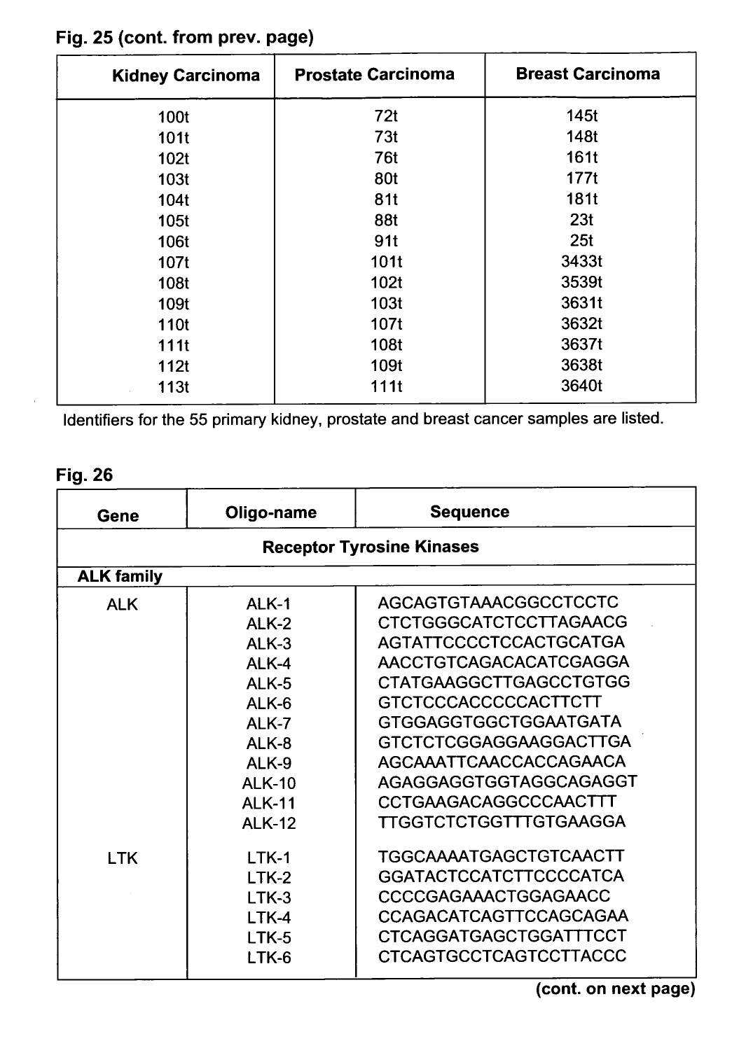

Somatic mutations clustering in the EGFR kinase domain (EGFR G719S, L858R, L861Q and others) have recently been reported for patients with Gefitinib-responsive NSCLC and were shown to enhance tyrosine kinase activity and sensitivity to Gefitinib in vitro (Paez et al., Science 304:1497-1500 (2004); Lynch et al., N Engl J Med 350:2129-2139 (2004); Pao et al., Proc Natl Acad Sci USA 101:13306-13311 (2004)). The present inventors found the EGFR G719S mutation to be heterozygously expressed in the colon cancer cell line SW-48 (FIG. 5C, FIG. 24 and FIG. 25). This demonstrates the existence of Iressa sensitivity-mediating mutations in cancers other than NSCLC and, in particular, suggests colon cancer as another potential indication for Gefitinib therapy.

Similar to EGFR L858R and Gefitinib, the KIT N822K mutation which the inventors confirmed in the AML cell line KASUMI-1 (Beghini et al., Hematol J, 3:157-163 (2002)) was reported to mediate sensitivity to Gleevec (Heinrich et al., J Clin Oncol 21:4342-4349 (2003)). The enhanced in vitro receptor activation shown for these EGFR and KIT mutations (30, 34) might be related to their location within the regulatory activation loop. In this respect, the sporadic variations FLT3 R849H, TEK A1006T, ABL G417E, ARG K450R, and TEC W531R which the inventors detected homo- or heterozygously in BM-1604, SK− MEL-2, MM-Leh, Caki-2, and Jurkat (FIG. 5C) are particularly intriguing as they are located in the activation loop as well. By inference, these mutations may also have a higher probability to modulate the TK catalytic activity and/or related signaling pathways within the respective tumor cell lines.

The 18 somatic mutations that the present inventors identified in intracellular juxtamembrane domains (FIG. 36) might affect functionally important elements that mediate downregulation of RTK activity. The in-frame deletion MET D981_E1027del as a result of exon 14 skipping, for instance, leads to the loss of c-Cbl E3-ligase binding, decreased ubiquitination, and prolonged ligand-dependent cell signaling in vitro and in vivo (Kong-Beltran et al.; Cancer Res 66:283-289 (2006)). While MET D981_E1027del was confirmed in the NSCLC cell line NCI-H596, its homozygous detection in breast and stomach cancer cell lines MDA-MB-415 and Hs746, respectively (FIG. 5C), provides evidence for its occurrence in tumor types other than the reported NSCLC (ibid; Ma et al.; Cancer Res 65:1479-1488 (2005)). Presuming enhanced sensitivity to anti-MET therapeutics that MET D981_E1027de1 was suggested to mediate (Kong-Beltran et al., 2006, supra), the findings disclosed herein extend the potential clinical relevance for this deletion.

The somatic mutations that the present inventors identified within the extracellular domain of two FGFR family members, FGFR1 P252S and FGFR4 Y367C (FIGS. 5C and 6B), possibly augment receptor activation by influencing ligand binding and receptor dimerization, respectively. The highly conserved FGFR1 P252 residue that the present inventors found to be heterozygously exchanged with hydrophilic serine in the melanoma cell line MeWo has previously been shown to be replaced by threonine in lung cancer (Davies et al., Cancer Res 65:7591-7595 (2005)) and arginine in patients with Pfeiffer syndrome (Muenke et al., Nat Genet. 8:269-274 (1994)). The crystal structure of the homologous activating FGFR2 mutant, FGFR2P252R, revealed the formation of 3 additional hydrogen bonds with complexed fibroblast growth factor 2 (FGF2). They were predicted to increase the receptor's affinity for its specific ligand as well as to allow binding of a different set of ligands (Ibrahimi et al., Proc Natl Acad Sci USA 98:7182-7187 (2001)). Since the hydroxy group of the P252-replacing serine residue in FGFR1 also has a high potential to form additional hydrogen bonds, the somatic FGFR1 P252S substitution may represent a gain-of-function mutation with analogous functional consequences as for FGFR2P252R. This is particularly intriguing in the context of studies demonstrating that blockage of FGFR1 or bFGF function was associated with suppressed proliferation and survival of melanoma cells (Wang & Becker Nat Med 3:887-93 (1997)).

The novel FGFR4 Y367C mutation was detected as a homozygous genotype in the breast cancer cell line MDA-MB-453, and the affected Y367 residue in the extracellular juxtamembrane domain is highly conserved throughout the FGFR family. Remarkably, homologous substitutions in FGFR1 (Y372C), FGFR2 (Y375C) and FGFR3 (Y373C) were shown to cause various osteogenic deficiency syndromes (Wilkie, Cytokine Growth Factor Rev 16:187-203 (2005)) through the formation of intermolecular disulfide bonds that force receptor dimerization and activation. Ligand-independent, constitutive receptor activation has been confirmed in vitro for FGFR1 Y372C (White et al., Am J Hum Genet. 76:361-367 (2005)) and FGFR3 Y373C (d'Avis et al., Cell Growth Differ 9:71-78 (1998)). Furthermore, the oncogenic potential of the FGFR3Y373C variant has been demonstrated and was suggested to contribute to tumor progression of multiple myeloma (Chesi et al.; Blood 97:729-736 (2001)). Thus, it is most likely that Y367C as the homologous FGFR4 variant also results in basal receptor activation, which strongly indicates an important role of this mutant in cancer.

### Nonsense Substitutions Abrogate Tumor Suppressor Activity

Downregulation of tumor suppressive activity is expected for the 2 nonsense substitutions that the present inventors detected in EPHB2 and CSK (FIG. 5C). Q722X-mediated truncation and kinase inactivation of EPHB2 in the two prostate cancer cell lines BM-1604 and DU-145 supports mutational inactivation to be involved in progression of prostate cancer as proposed by Huusko et al. They showed suppressed growth and colony formation of DU-145 cells upon reconstitution with functional EPHB2 (Huusko et al., Nat Genet. 36:979-983 (2004)). The heterozygous CSK Q26X nonsense substitution detected in the colon cancer cell lines DLD-1 and HCT-15 is consistent with reduced protein levels of this negative regulator of SRC-family kinases that were reported for ˜60% of human colon cancer cases with elevated SRC activity (Rengifo-Cam et al., Oncogene 23:289-97 (2004)). These data indicate a significant role of CSK nonsense mutations in the development and/or progression of colon carcinoma and therefore strongly suggest the inclusion of SRC kinase inhibitors in the therapeutic regimen of this prevalent malignancy.

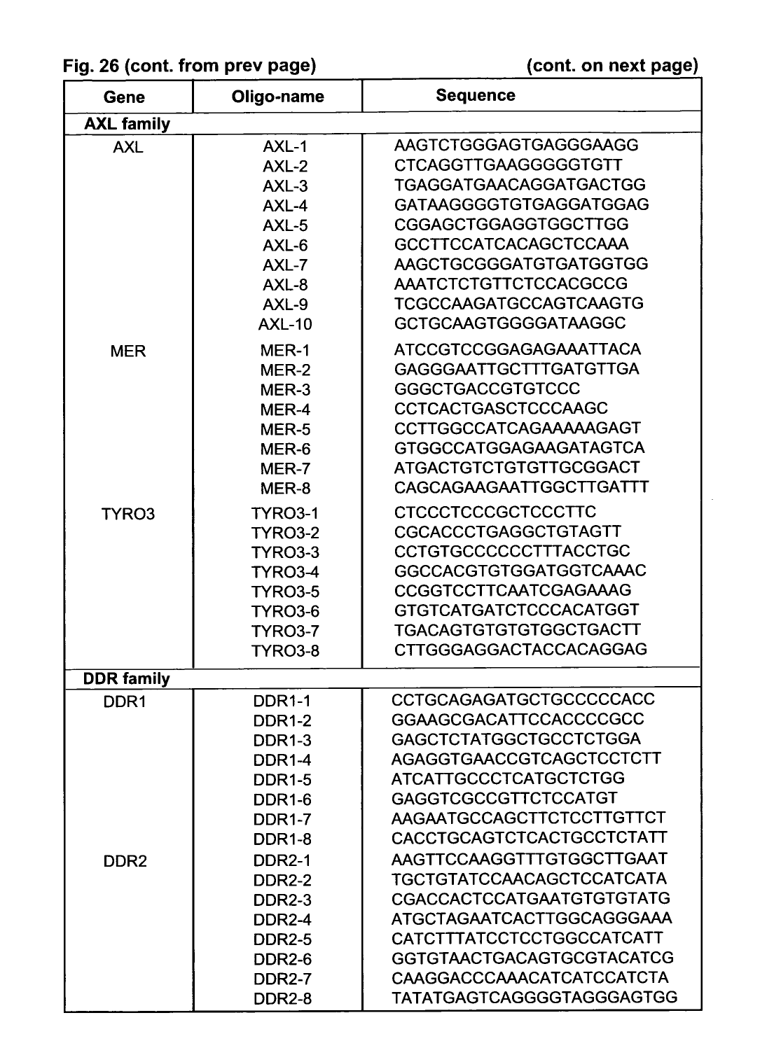

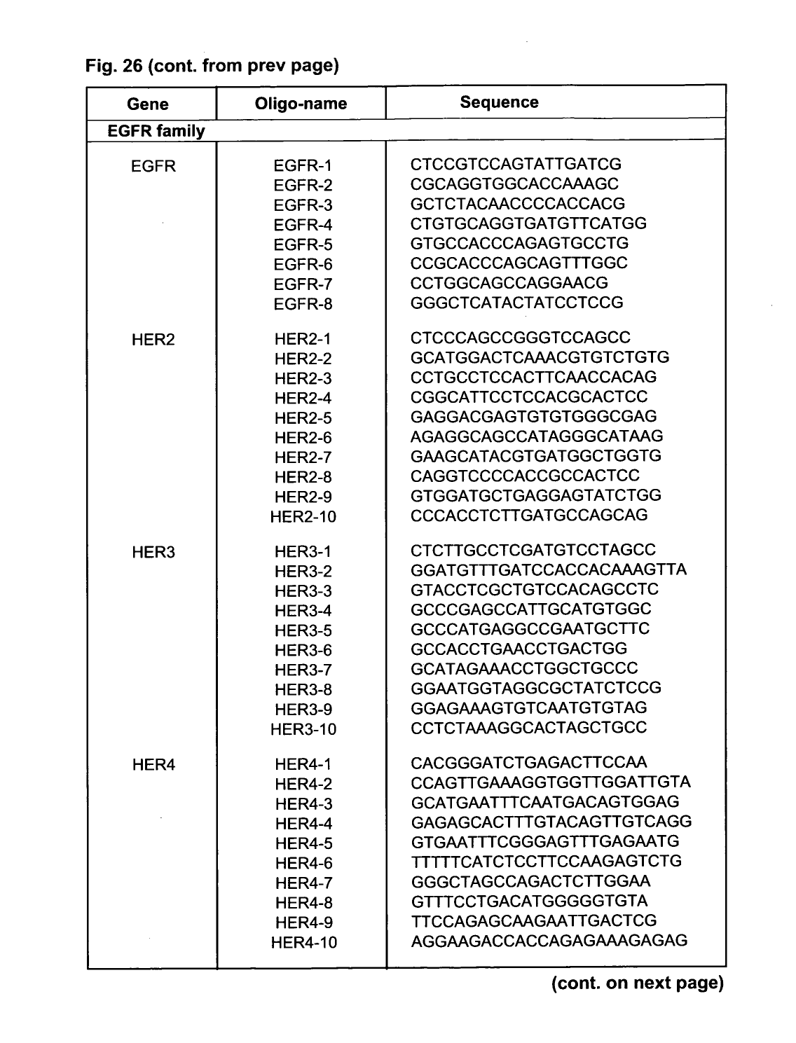

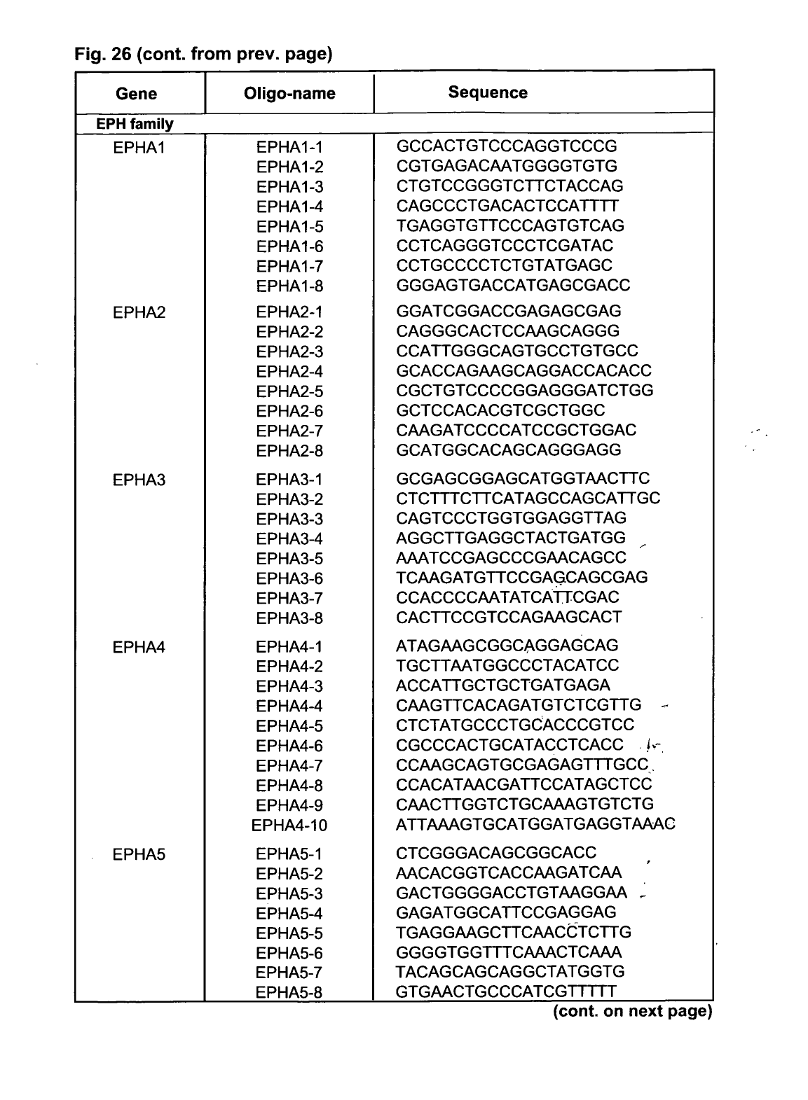

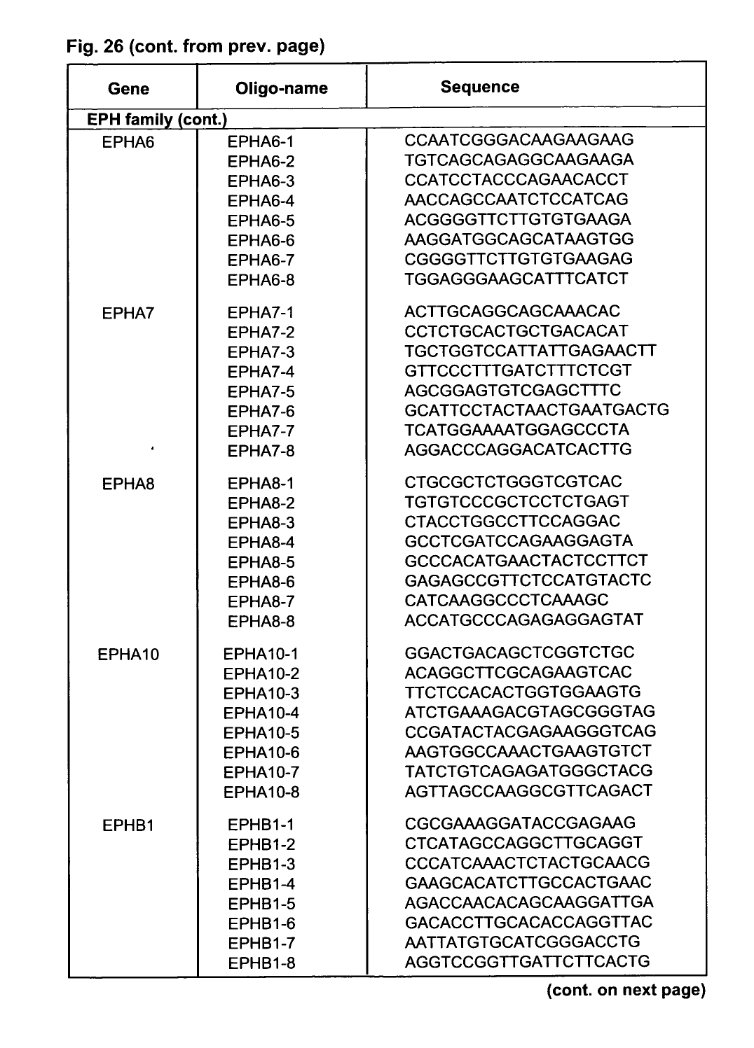

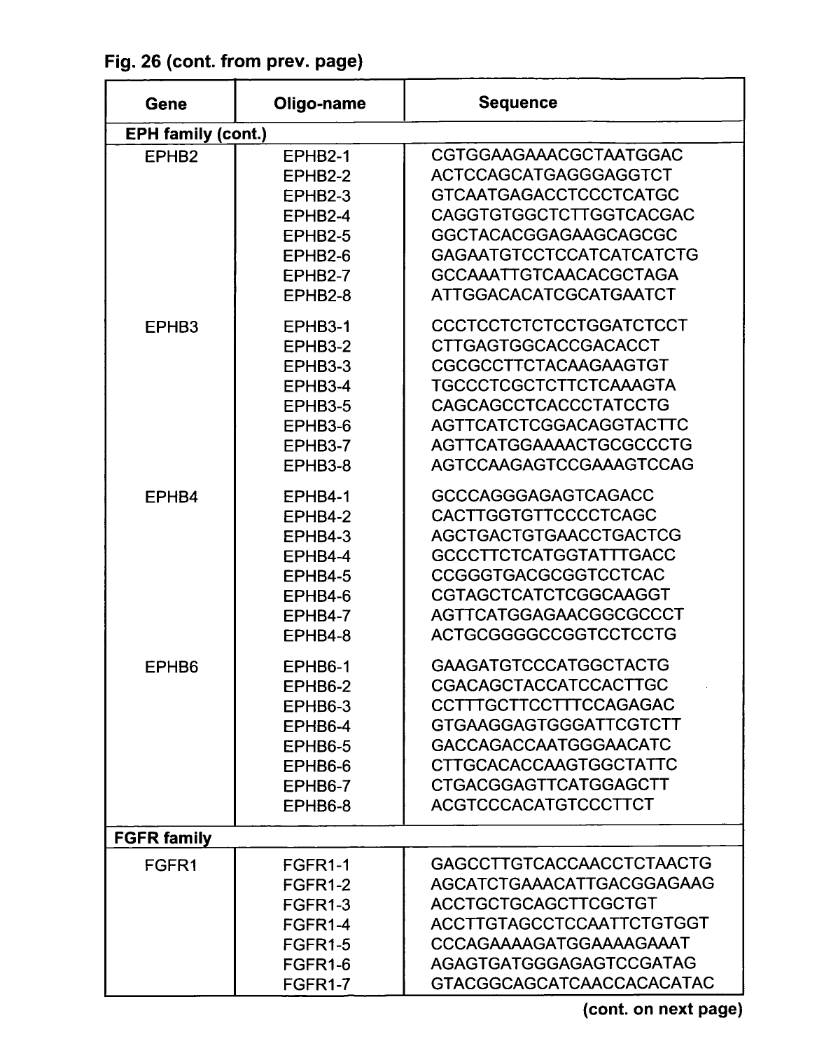

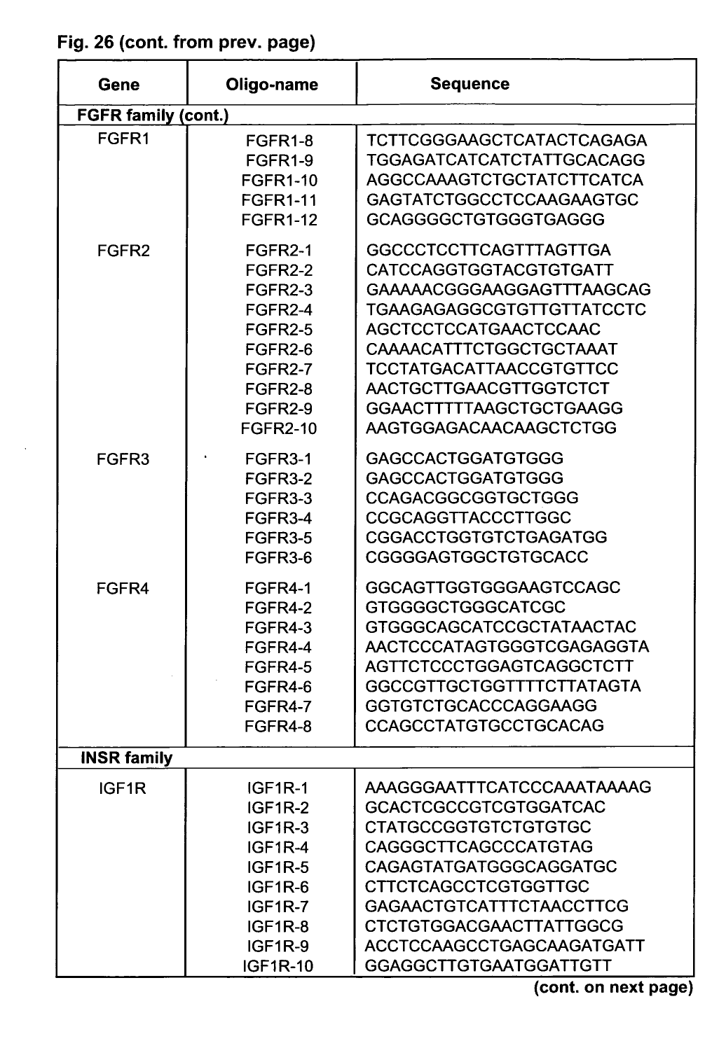

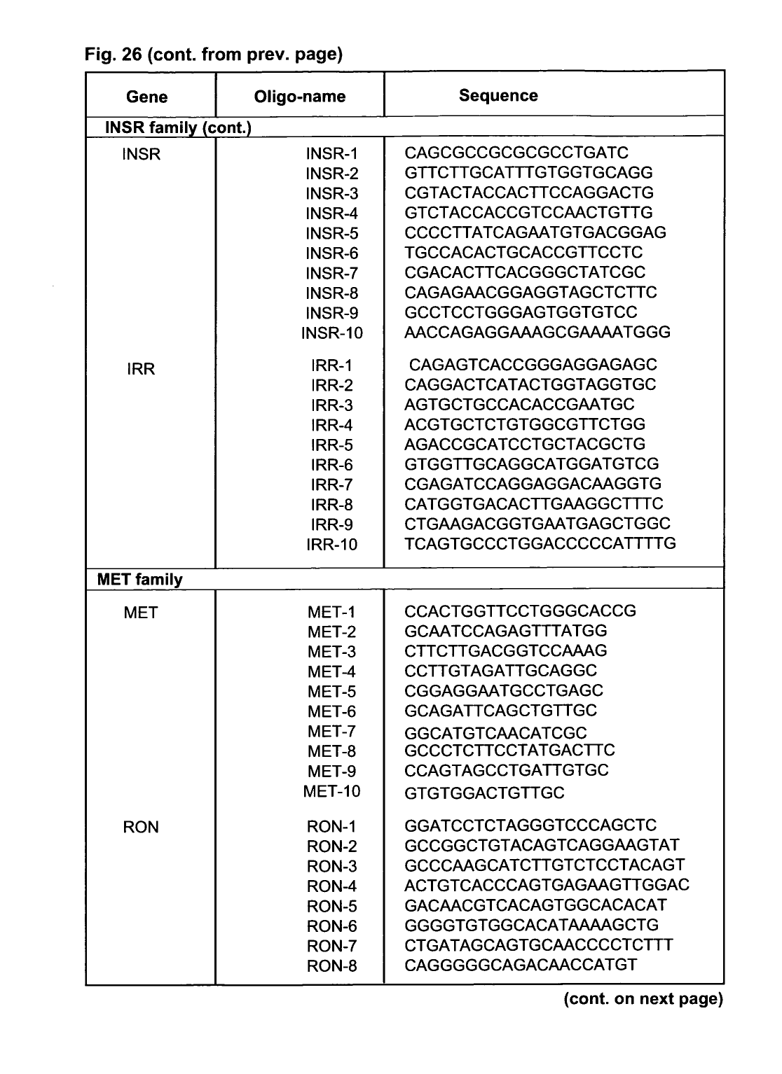

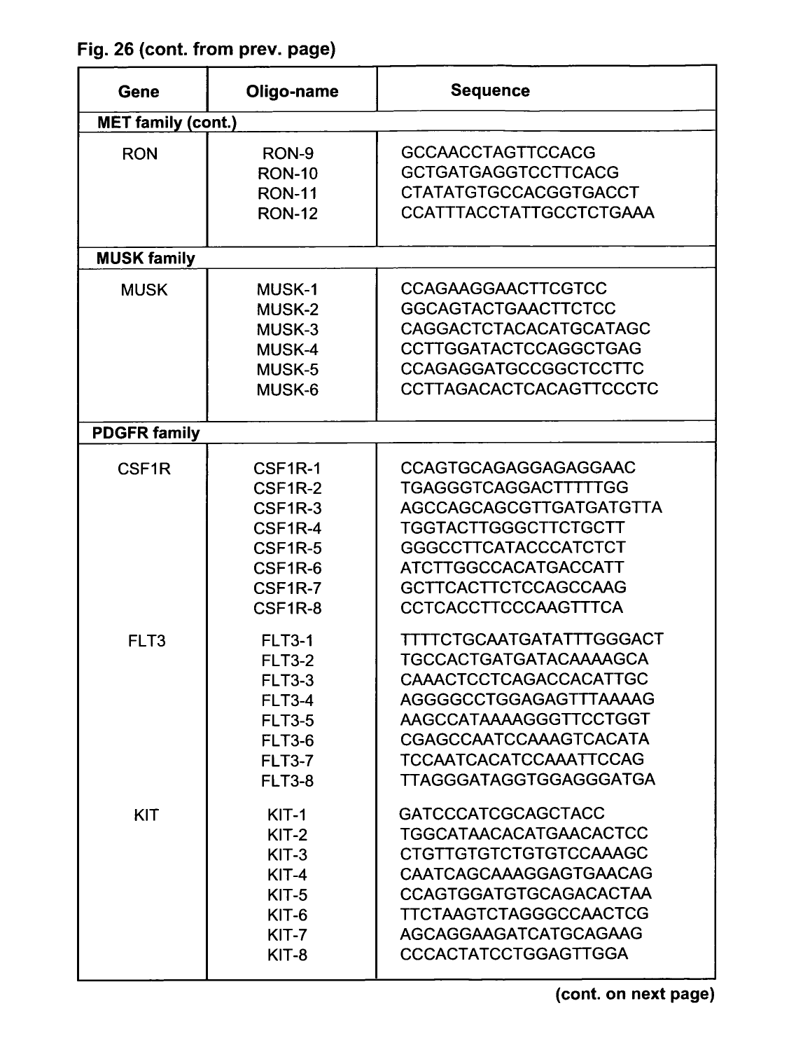

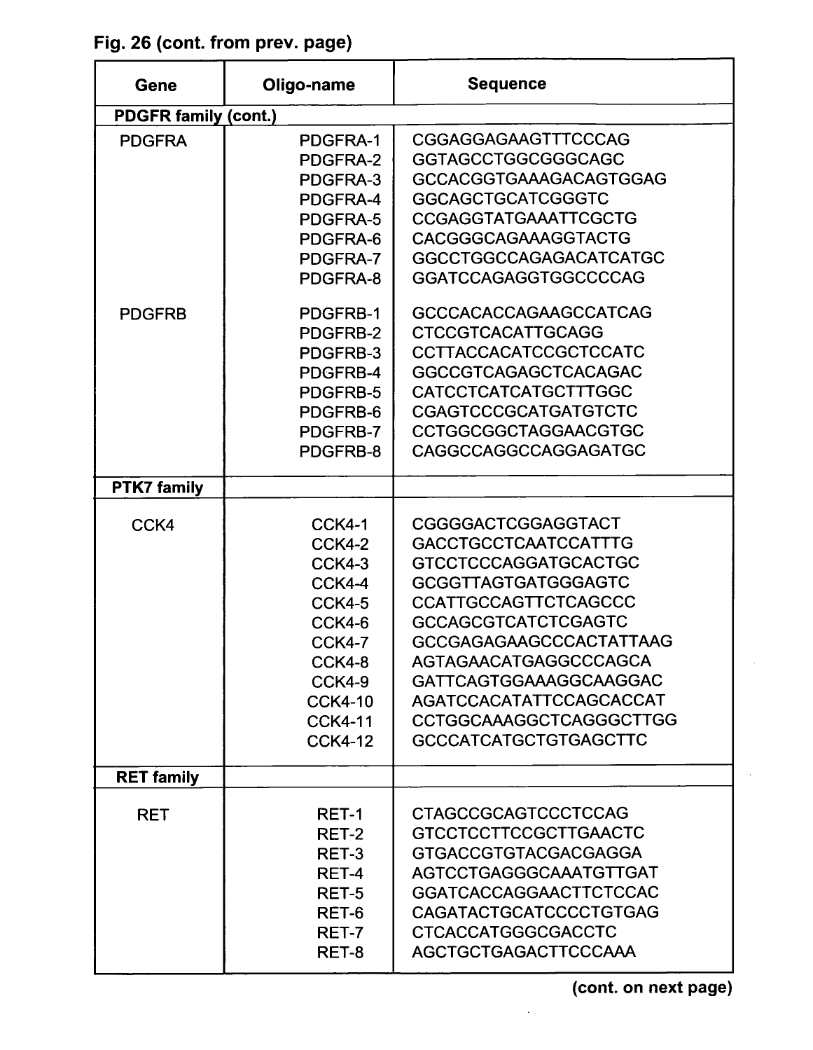

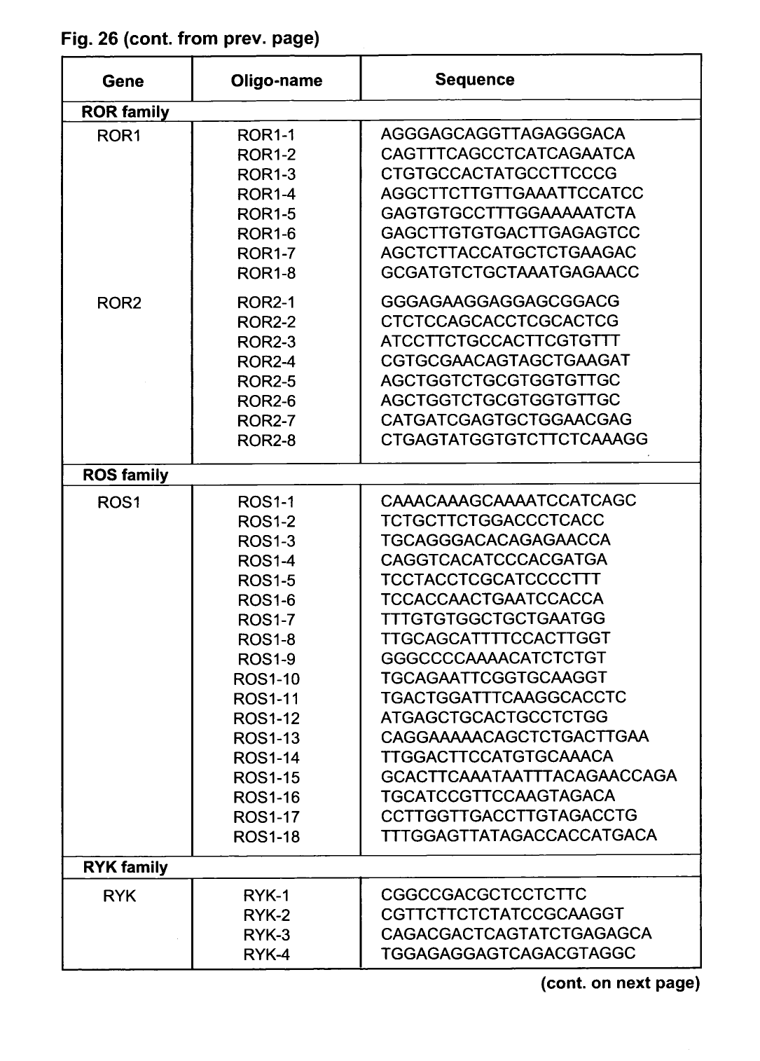

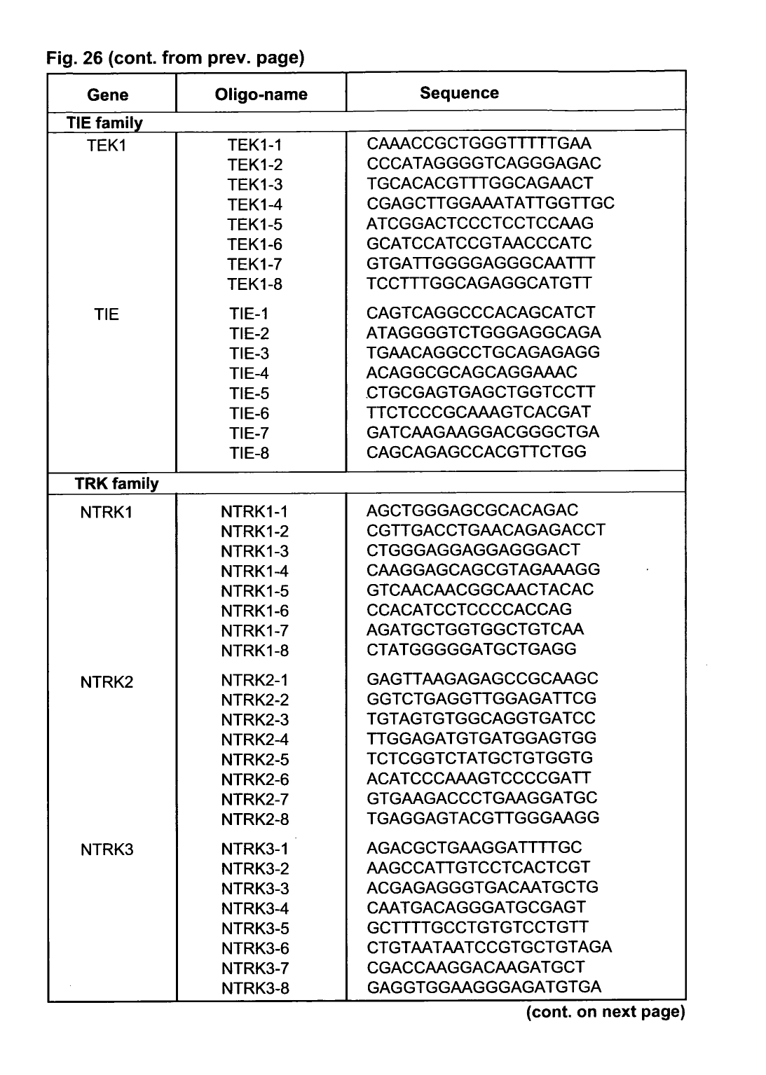

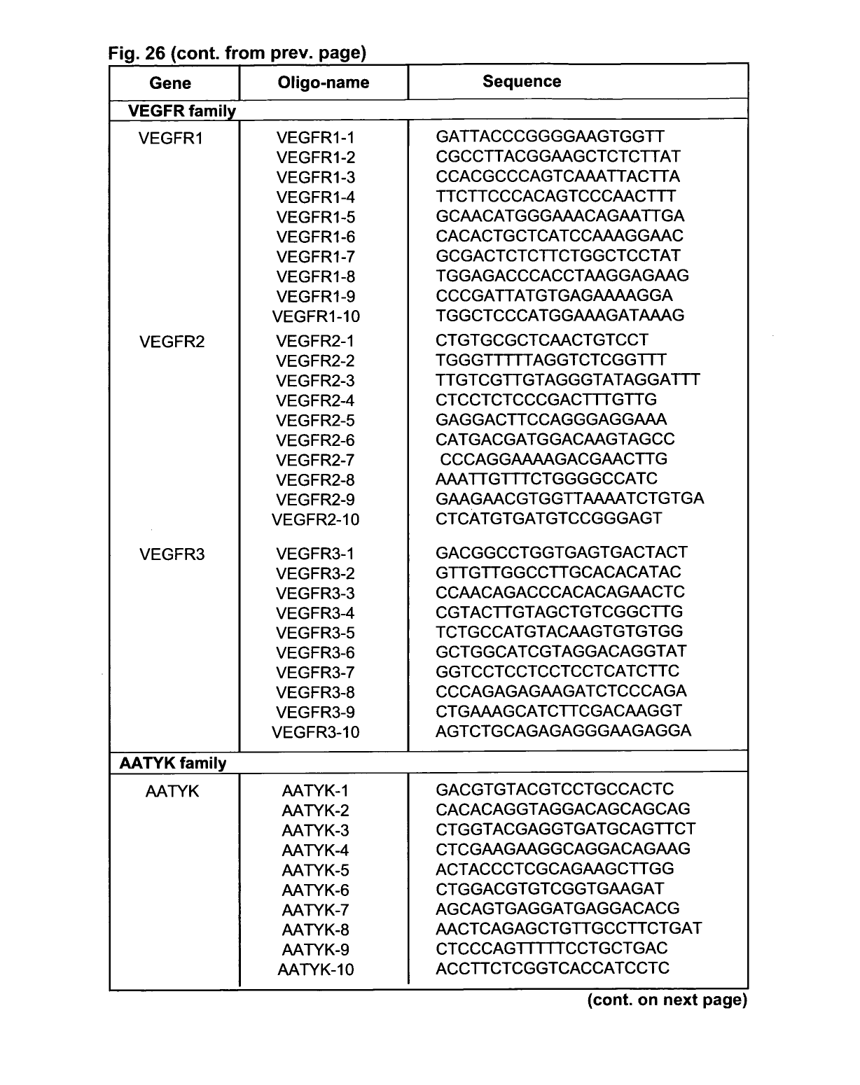

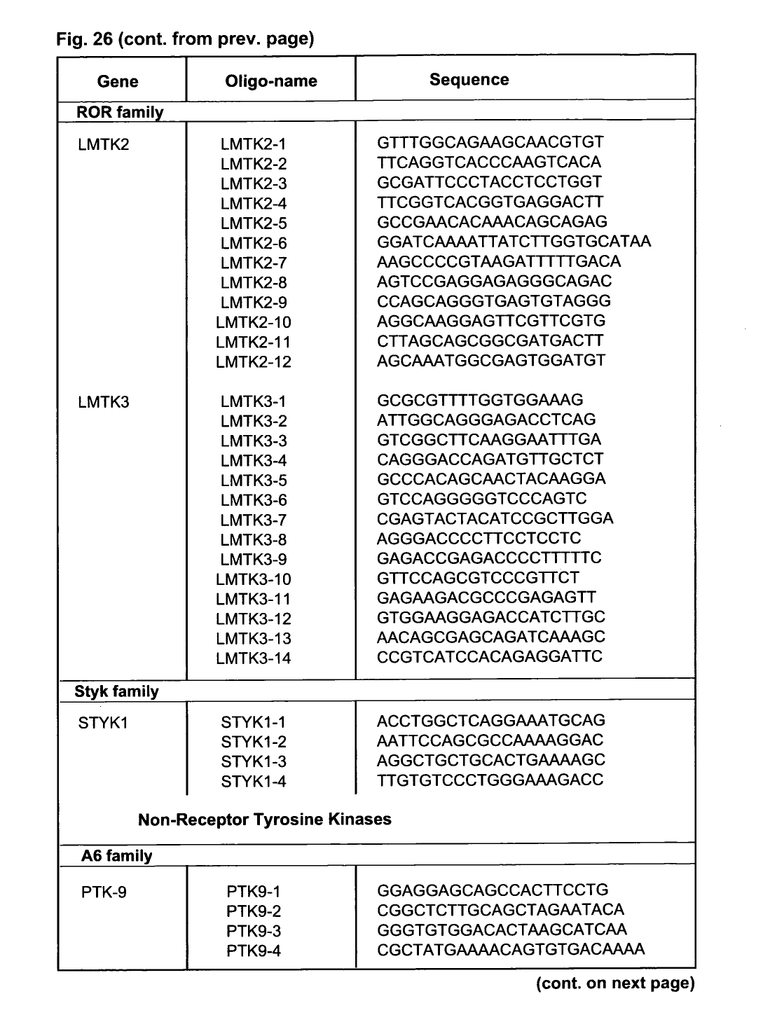

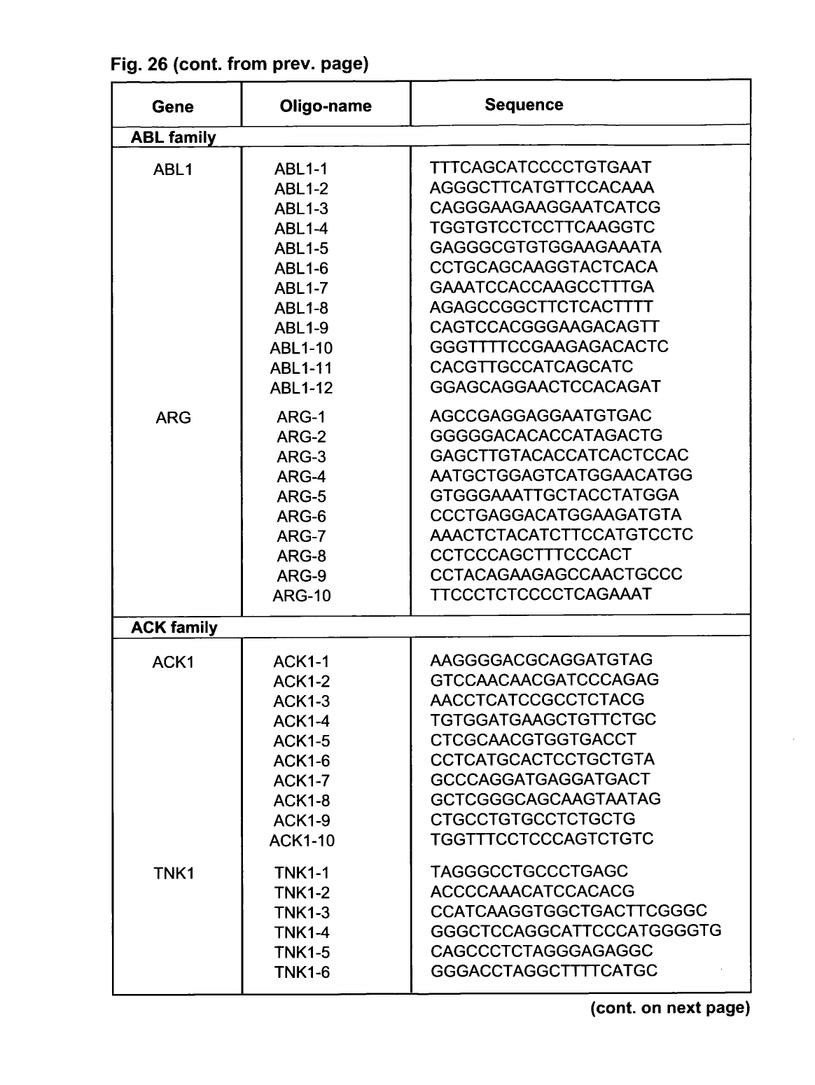

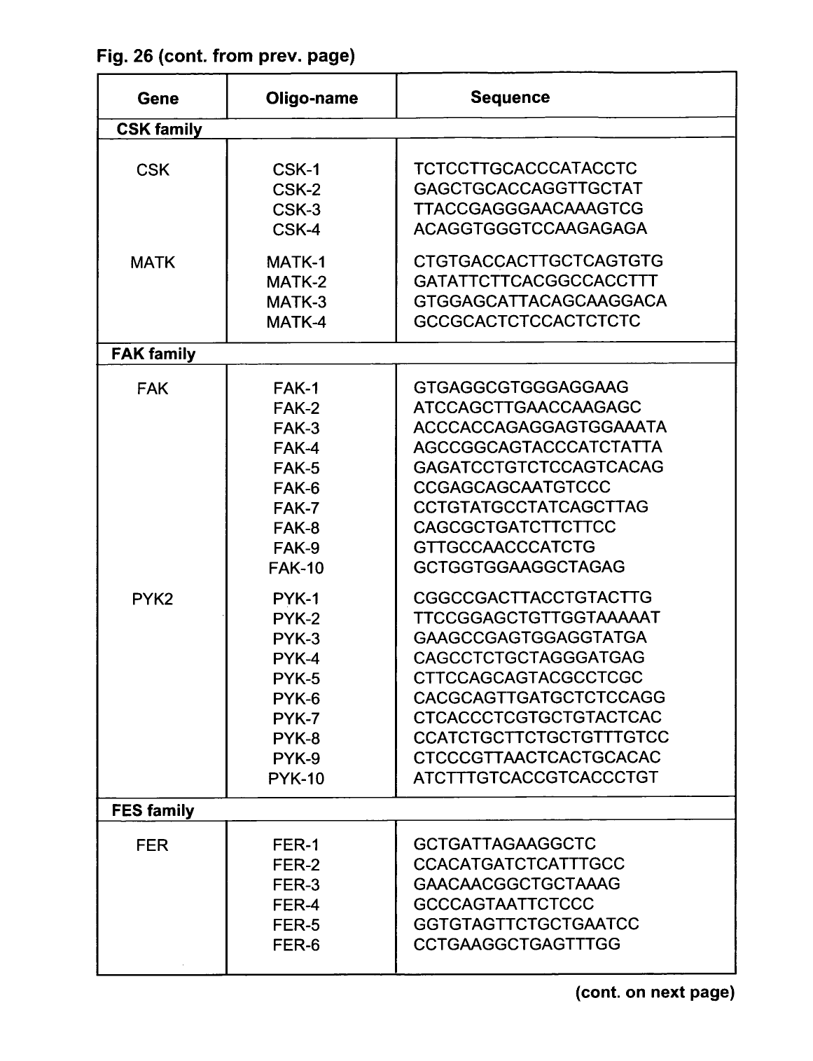

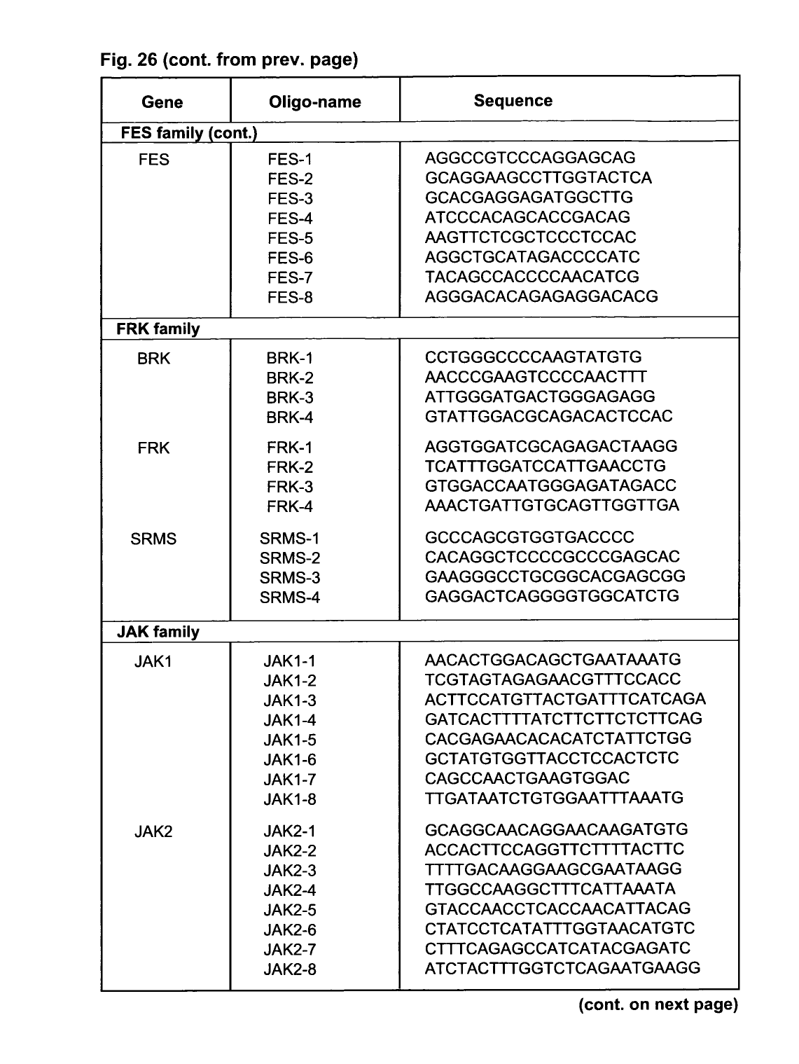

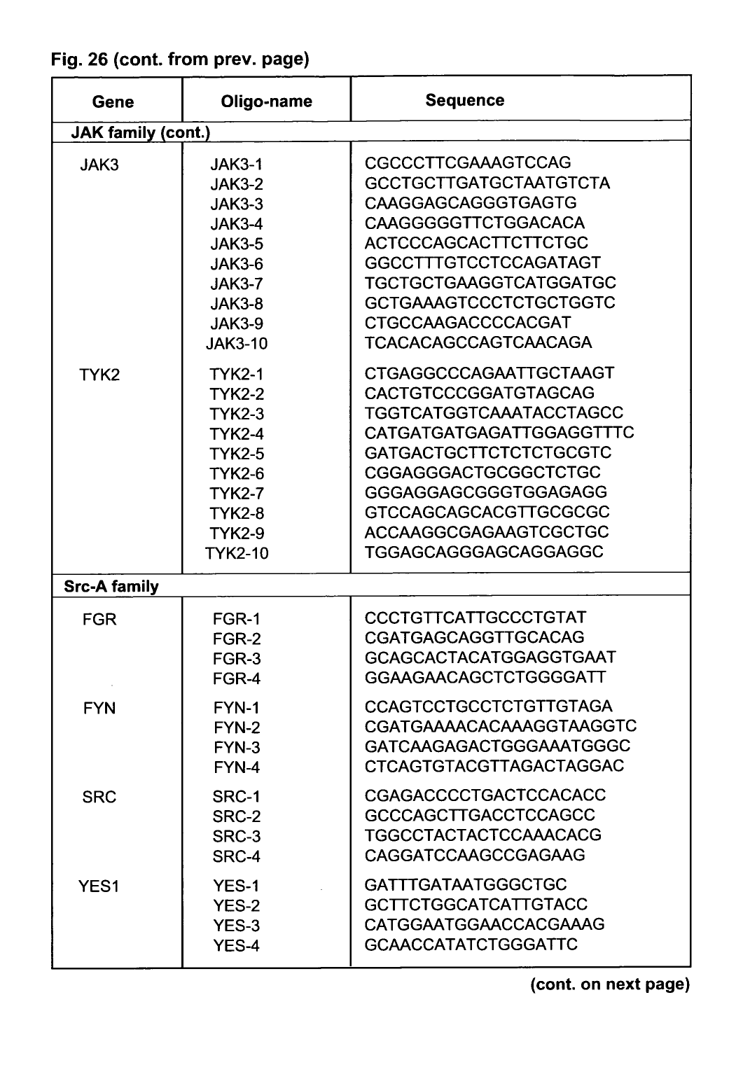

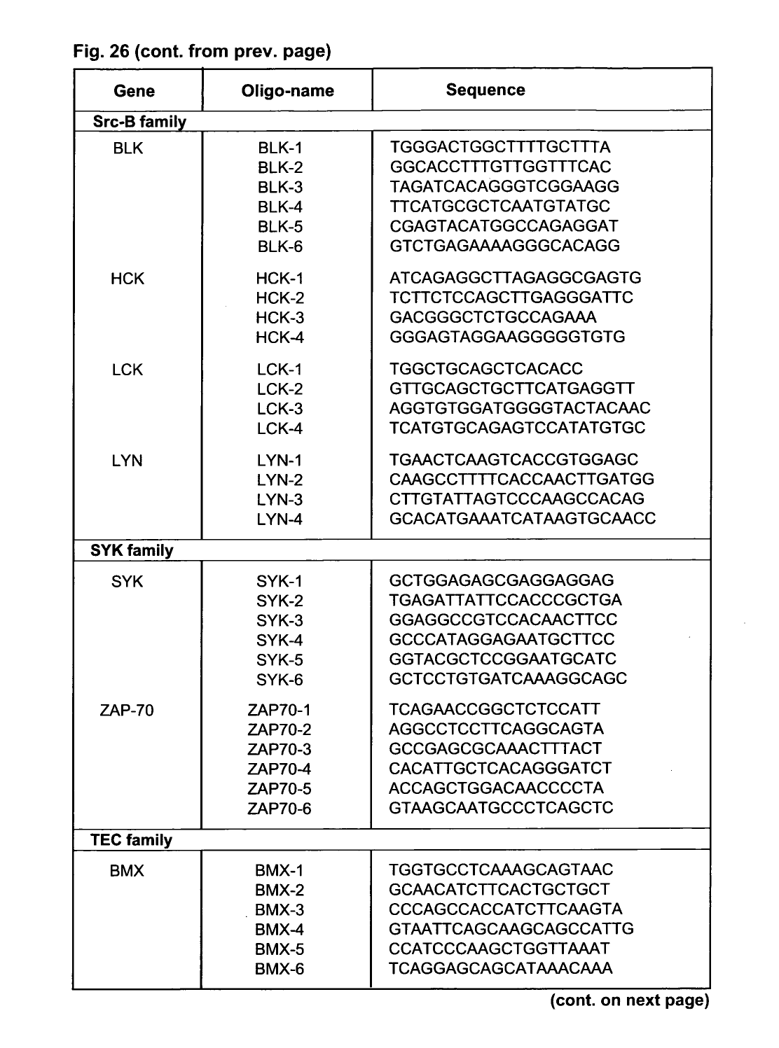

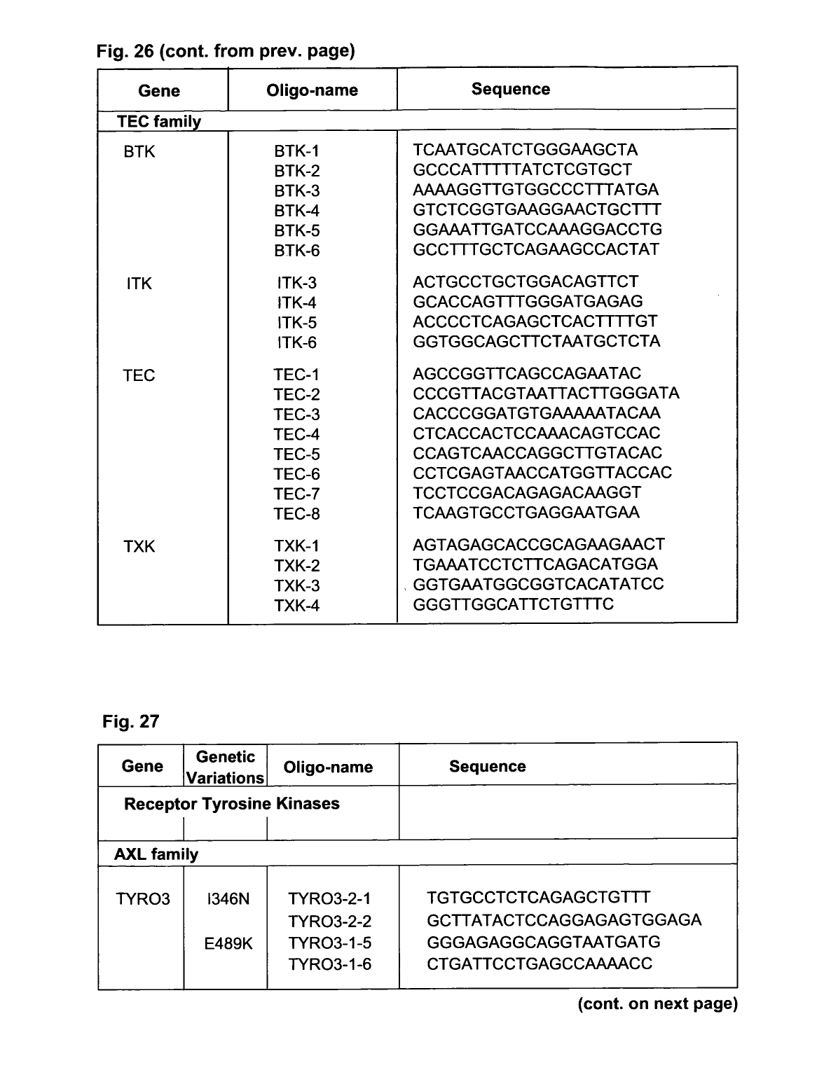

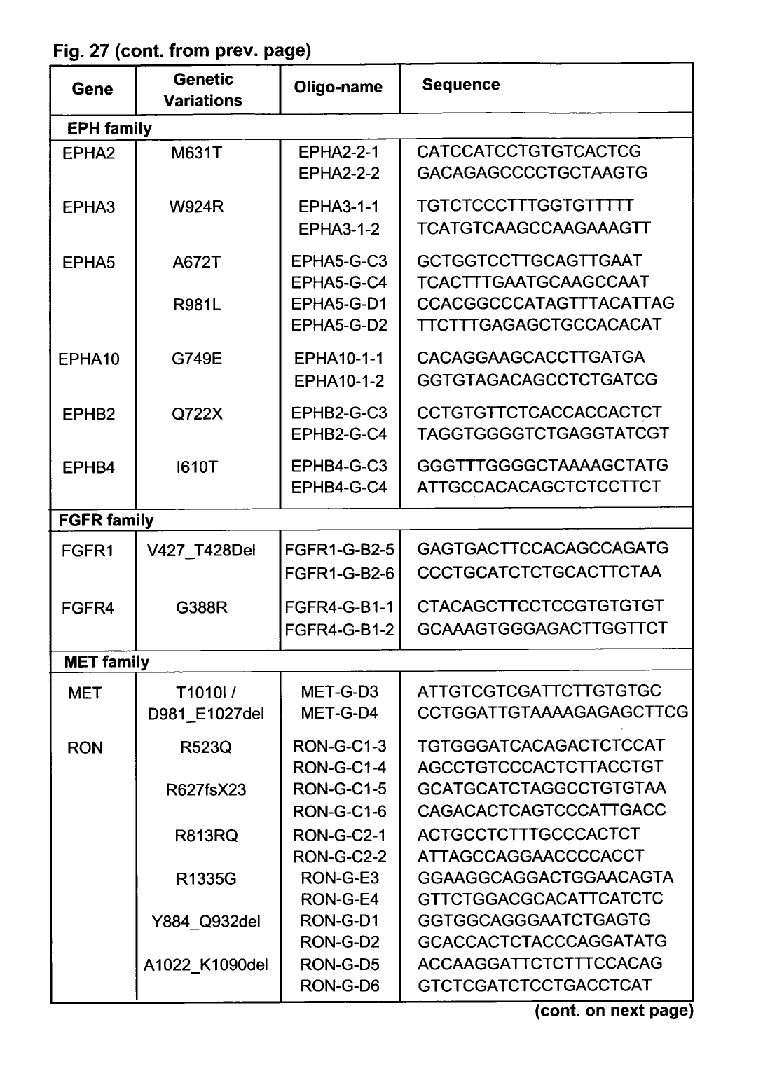

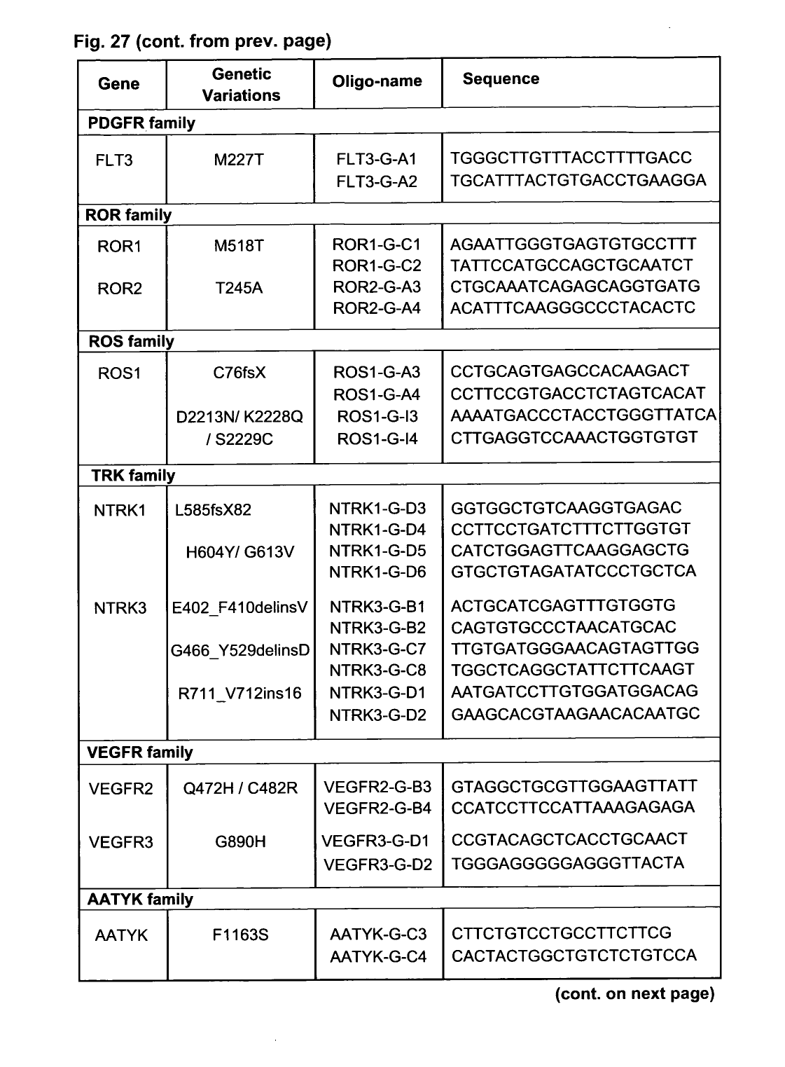

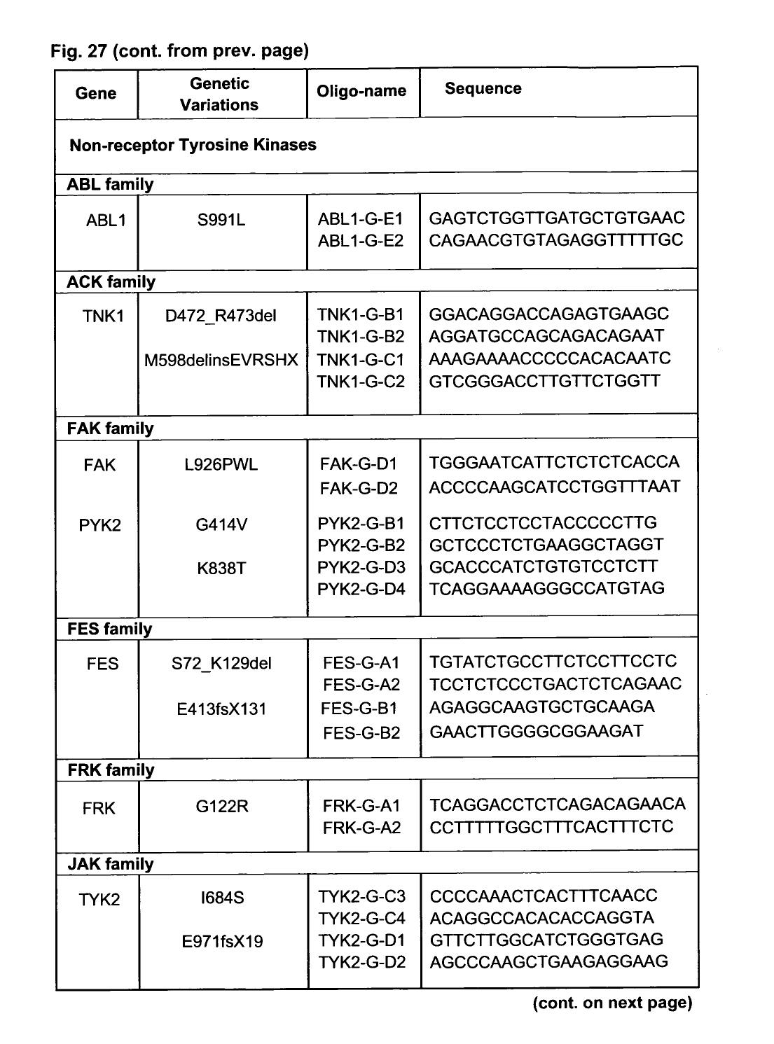

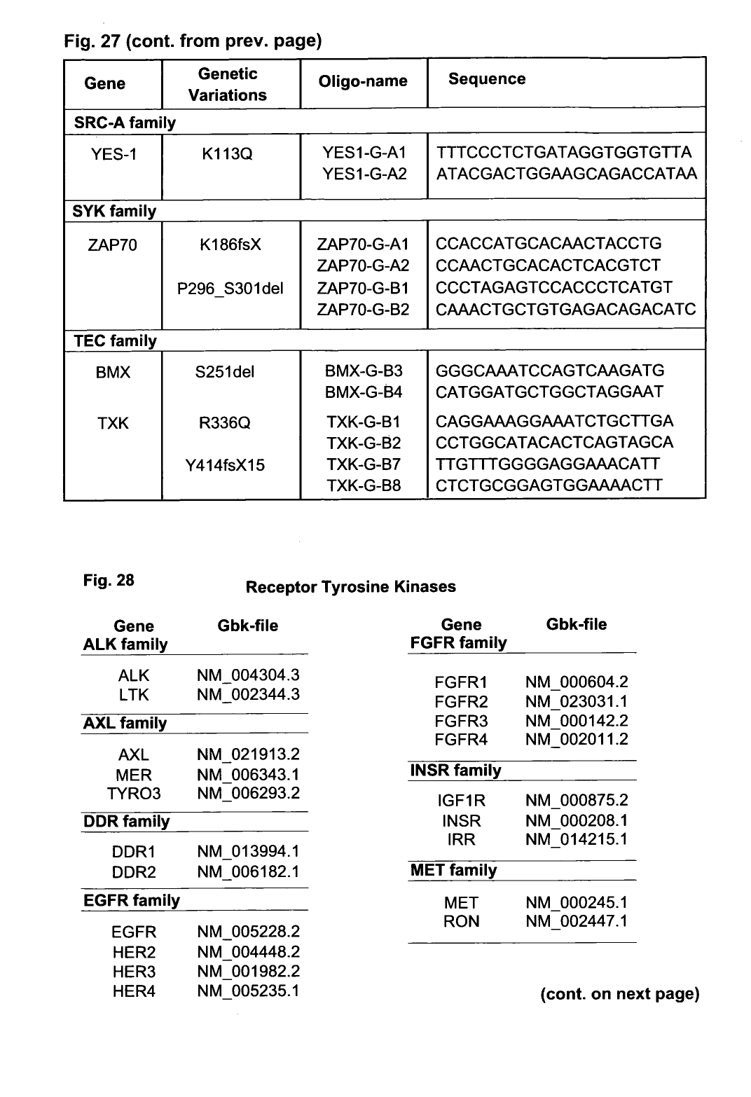

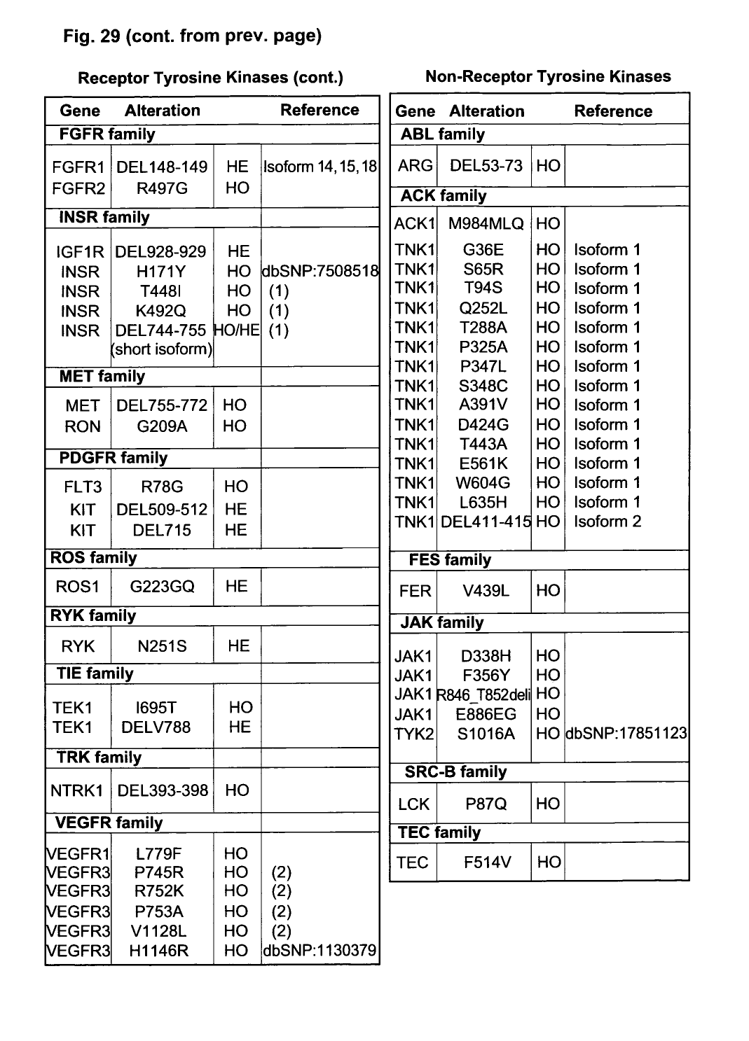

The examples discussed above represent only a partial extract of our overall data. Other genetic alterations affecting less investigated PTKs such as members of the AATYK-, DDR-, EPH-, ROR-, ROS- or FRK families, as well as tyrosine kinases that more recently captured scientific attention such as HER3 or ACK1, have been found (FIGS. 25-29). Their identification shall support novel functional investigations towards the understanding of the therapeutic value of these kinases.

### Low Redundancy of PTK Gene Mutations in Human Tumors

In agreement with results from previous studies (Stephens et al., 2005, supra; Davies et al., 2005, supra; Bignell et al., 2006, supra; Stephens et al., 2004, supra; Bardelli et al., 2003, supra; Thomas et al., 2007, supra; Greenman et al., 2007, supra; Sjoblom et al., Science 314:268-274 (2006)), the analysis of 254 cancer cell lines and additional primary tumors presented here indicates that mutational patterns might be quite unique for the majority of human tumors, and that the frequency of specific somatic mutations in PTKs is low. Data mining of public databases and the literature revealed that only 9 of all sporadic alterations identified in our study have been described before (FIG. 37). Among them are KIT N822K and VEGFR1 R781Q as the 2 only ones that were picked up in the currently most comprehensive mutational kinome analysis of human cancer samples (Greenman et al., 2007, supra). The low redundancy of somatic mutations is furthermore reflected by the non-recurrence that that the present inventors found for 206 of the 256 mutational events within our panel of tumor cell lines.

Consistent with this picture, none of the 7 somatic representatives of our exemplary subset of non-conservative and more frequent alterations found in at least one breast-, kidney-, or prostate cancer cell line could be detected in any of the 165 primary breast-, kidney- and prostate cancer specimens (FIG. 36). In fact, two genetic alterations, YES K113Q and TYRO3 E489K, were found in blood controls and therefore must be considered rare germline alterations.

Despite the low redundancy of individual mutations, 70 of the tyrosine kinase genes turned out to carry at least one somatic mutation. Although most of our mutations require further experimental evaluation to determine their cancer relevance and in some cases may turn out to represent “passenger” rather than “driver” mutations, this broad incidence of sporadic alterations underscores the central importance of the entire PTK family in oncogenesis. Moreover, it provides further compelling support for the development of multi-targeted kinase inhibitors or combination of complementary therapeutics as cancer treatments which may be adapted to the pathological and genetic parameters of an individual patient. The extensive characterization of established tumor cell lines with respect to transcriptional profiles of genetic variations in this currently most promising cancer target family will aid in the selection of suitable cell systems, data interpretation and target validation, and thereby support preclinical development of novel targeted cancer drugs.

### Cell Culture, Plasmids

HEK293, Jurkat E6.1, HuH7, HepG2, MCF-7 and MDA-MB-231 cells were purchased from ATCC (Manassas, Va.). HEK 293, HuH7 and MCF-7 were maintained in DMEM (high glucose) medium supplemented with sodium pyruvate and 10% FCS. Jurkat E61 and MDA-MB-231 were maintained in RPMI supplemented with L-glutamine, sodium pyruvate and 10% FCS. HepG2 was maintained in MEM supplemented with non-essential amino acids, L-glutamine, sodium pyruvate and 10% FCS. All cell culture reagents were from Invitrogen (Carlsbad, Calif.) unless otherwise stated.

Full-length cDNAs encoding TEC were amplified by PCR from a human placenta cDNA library and subcloned into pcDNA3 (Invitrogen, Carlsbad, Calif.). Generation of mutants was performed using QuikChange Site-Directed Mutagenesis Kit from Statagene (La Jolla, USA). The expression construct for human Ack1 (pXJ40-Ack1-Flag) was a gift from Edward Manser (IMCB, A*STAR, Singapore).

### Sample Preparation:

Fifty seven adjacent normal tissue were obtained from resected livers of patients from the National University Hospital (patient's consent for collection of tissue was obtained prior operation). The tumor and normal liver tissues were visually separated. Both tumor and normal tissue were cut into small pieces and flash frozen in liquid nitrogen immediately after being harvested from patients. The frozen tissues were later stored in −80° C. RNA extraction from frozen tissue was carried out by TriZol method as described previously (Chomczynski, P., & Sacchi, N., Nat Protoc 1:581-5 [2006]).

mRNA Purification

mRNA was purified from total RNA (50 μg per sample) using the Oligotex mRNA kit (Qiagen, Valencia, Calif.) performed according to manufacturer's protocol. The resulting mRNA from the Oligotex columns was eluted with two volumes of 50 μL of the elution buffer supplied in the kit. To purify and concentrate the eluant, 2 μL of pellet paint (Merck, Darmstadt, Germany) and 10 μL of 3M sodium acetate were first added to enhance visualization of the produce before precipitating overnight with 200 μL of absolute ethanol at −20° C. The resultant mRNA was pelleted by spinning at 13,000 rpm for 30 min and subsequently washed with another volume of 80% ethanol. The final precipitate was air-dried and re-dissolved in 10 μL of RNAse-free water.

First Strand cDNA Synthesis

The purified mRNA (4 μL) from above was mixed gently with 1 μL of OligoDT15 primer (100 μM, Roche, Basel, Switzerland) in a 1.5 mL microcentrifuge tube and incubated at 70° C. for 2 min. After cooling on ice, 15 μL of reverse transcription mix containing 4 μL of 5×RT buffer, 2.4 μL of MgCl₂, 1 μL dNTP (10 mM), 1 μL RNase inhibitor, 1 μL ImProm-II RT (Promega) and 5.6 μL of water was added. The reaction was maintained at 42° C. for 1 h and was quenched with 75 μL of TE buffer. The resultant cDNAs were purified with QiaQuick PCR purification kit (Qiagen).

### Sequencing and Mutational Analysis:

Primers for PCR amplification of cDNA samples (and sequencing) were designed using Primer3 program (http://www-genome.wi.mitedu/cgi-bin/primer/primer3_www.cgi), and were synthesized by Proligo (SigmaAldrich, Singapore). PCR reactions were optimized as previously described for FGFR4. Direct sequencing was done using a 96 capillary automated sequencing apparatus (ABI 3730XL). Sequence traces were assembled and analyzed to identify potential genomic alterations using the Mutation Surveyor software package (SoftGenetics, State College, Pa.). The entire coding sequence of FGFR4 was aligned to the NCBI reference sequence (NM_{—}002011.2) and identified alterations were compared to known mutations in the literature (in publications) or public databases such as NCBI SNP database (http://www.ncbi.nlm.nih.gov), the Ensemble Genome Browser (http://www.ensembl.org), the UniProtKB/Swiss-Prot database (http://ca.expasy.org) and KinMutBase (http://bioinf.uta.fi/KinMutBase/main_frame.html).

### Quantitative Real-Time PCR

Quantitative RT-PCR was carried out using an Applied Biosystems 7300 Real-time PCR system (ABI, Foster City, Calif.) with pre-optimized TaqMan Gene Expression Assay for human FGFR4 and human GAPDH as the housekeeping control. The thermal cycling condition included an initial denaturation step at 95° C. for 10 min, followed by 40 cycles at 95° C. for 15 s and 60° C. for 60 s. The samples were prepared in triplicate with 4 μL of prediluted cDNA (2-10 fold) samples each. Data were obtained as an average CT value, and subsequently normalized against GAPDH endogenous control as ΔC_{T}. Expression changes in FGFR4 transcripts between the normal and the corresponding tumor tissue were expressed as fold change using 2̂(difference in ΔC_{T} between pairs).

### Ligand Stimulation Assay

HepG2 or HuH7 cells were seeded into 96-well plates and allowed to attach overnight. They were serum-starved for 24 h before addition of heparin. Two hours later, FGF19 (R&D Systems, Minneapolis, Minn., 50-100 ng/mL final concentration) was administered. After 8 hours, aliquots of the supernatant were harvested for AFP ELISA assay as described below. Similar FGF19 stimulations were performed in 10 cm dishes for immunoblot detection of phospho-FRS2α.

### Immunoprecipitation, Immunoblot Assay

All fractions collected were assayed for protein concentration using a BCA protein assay kit (Pierce, Rockford, Ill.). Lysates were pre-cleared by centrifugation at 13 000 r.p.m. for 10 min at 4° C. For immunoprecipitation, supernatants were diluted with an equal volume of HNTG buffer (Seedorf, K., et al., J Biol. Chem. Jun. 10; 269(23):16009-16014 (1994)) and subsequently immunoprecipitated using the respective antibodies and 20 μl of protein A/G-Sepharose for 4 h at 4° C. Precipitates were washed three times with 0.5 ml of HNTG buffer, suspended in SDS sample buffer and boiled for 3 min.

For the immunoblot assay, sample proteins (30-50 μg) or the immunoprecipitated samples were resolved by denaturing electrophoresis using 7.5 SDS-PAGE and transferred to nitrocellulose membranes for 2 h at 5 V using Trans-Blot SD Semi-Dry Transfer Cell (Bio-Rad). Immunodetection was by chemiluminescence (SuperSignal West Dura Extended, Pierce) using specific antibodies diluted in PBS with 0.05% (v/v) Tween 20 and 5% (w/v) powdered milk. Anti-phospho-FRS2α anti-HA, anti-phospho-MAPK, anti-MAPK, anti-phospho-Stat3, anti-Stat3 were from Cell Signaling Technology (Beverly, Mass.); anti-Ack1, anti-myc and anti-FGFR4 were from Santa Cruz Biotechnology (Santa Cruz, Calif.); anti-β-actin controls and anti-TEC were from Abcam (Cambridge, UK); anti-4G10 and anti-TYK2 were from Upstate (Lake Placid, N.Y., USA); anti-Hsp60, anti-Flag were from Sigma (St Louis, USA). Secondary anti-mouse and anti-rabbit horseradish peroxidase conjugated secondary antibodies (Pierce) were used at 1:10000 dilution.

### AFP ELISA Assay

Supernatants from the respective FGF19 stimulation and siRNA experiments were subjected to AFP ELISA assay using the DELFIA hAFP kit (Perkin Elmer, Boston, Mass.), performed according to manufacturer's protocol. The readout was converted to concentration (ng/mL) using the standard curve derived from the solutions provided in the kit. AFP production by the cells was subsequently normalized to the number of cells in each well. The cell number was determined by ATP bioluminescence Cell-Titer Glo assay (Promega, Madison, Wis.) using protocol as described by the manufacturer.

Gene Silencing by siRNA

HuH7 cells were grown on 24-well plate to 50% confluence before transfection with siRNA (small interfering RNAs). Custom-made ON-TARGETplus siRNA designed for silencing FGFR4 (Accession number: NM_{—}002011) expression was purchased from Dharmacon. A microcentrifuge tube containing 1.3 μL of 20 μM siRNA and 40.2 μL complete growth medium was prepared (Tube A). Simultaneously, another tube containing 1 oligofectamine (Invitrogen) and 7.5 μL growth medium was also prepared (Tube B). Both tubes were incubated at room temperature for 5 min before combining the contents and left to stand for another 20 min. Next, each well of HuH7 cells was replaced with fresh serum-free medium (200 μL). The combined volume of the siRNA transfection mix (50 μL) was added to the well and incubated at 37° C. After 4 h, the samples were loaded with another 250 μL of growth medium containing 20% serum. Immunoblot and AFP ELISA assays were subsequently performed after 72 h incubation of the cells with the FGFR4 silencing complex.

c-fos Gene Reporter Assay

HEK-293 cells (2×10⁴/96-well) were transiently transfected with 0.2 μg of the pfos/luc reporter plasmid (Yamashita et al., Blood 91, 1496-1507 (1998)) and 0.1 μg of expression plasmids for TEC or its mutants. 24 hours after transfection luciferase activity was measured with the use of the dual luciferase assay system (Promega, Madison, Wis.).

### Ubiquitination Assay

3.5×10⁶ Hek 293 cells were seeded on 10 cm dish. Cells were co-transfected with 7.5 μg Myc-tagged Ubiquitin and 10 μg Flag-tagged Ack1, Ack1 S985N, Mop1 and EphA5, using Lipofectamine™ 2000 (Invitrogen, San Diego, Calif.) according to the manufacturer's instructions.

20 hours post transfection, cells are incubated with 10 μM MG132 (Sigma, St Louis, Mo.) for 8 hours and lysed by RIPA buffer. 500 μg of protein lysate were used for immunoprecipitation with 2 μg anti-myc antibody at 4 degrees Celsius overnight. IP samples were washed and denatured with SDS-lysis buffer (50 mM Tris-HCl pH 6.8, 100 mM DTT, 2% SDS) and separated on a 7.5% SDS-PAGE gel. Co-IP proteins were detected using anti-Flag antibodies.

### MG132 Treatment

1×10⁶ HepG2 cells were seeded on 6 well plate the day before treatment. Cells were incubated with 10 μM MG132 (Sigma, St Louis, Mo.) and harvested according to time course. 30 μg cell lysate was separated with a 7.5% SDS-PAGE gel.

The listing or discussion of a previously published document in this specification should not necessarily be taken as an acknowledgement that the document is part of the state of the art or is common general knowledge.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” etc. shall be read expansively and without limitation, and are not limited to only the listed components they directly reference, but include also other non-specified components or elements. As such they may be exchanged with each other. Additionally, the terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed.

Other embodiments are within the following claims. In addition, where features or aspects of the invention are described in terms of Markush groups, those skilled in the art will recognize that the invention is also thereby described in terms of any individual member or subgroup of members of the Markush group.

1. An isolated, enriched, or purified nucleic acid molecule encoding a mutant of a protein kinase polypeptide, wherein the protein kinase polypeptide is selected from the group consisting of FGFR4, FGFR1, Tyro3, TEC, CSK and Ack1, and
wherein the mutant of the protein kinase polypeptide encoded by the nucleic acid molecule comprises at least one mutation selected from the group consisting of FGFR4 Y367C (SEQ ID No: 133), FGFR1 P252S (SEQ ID No: 129), Tyro3 S531L (SEQ ID No: 257), Tyro3 P822L (SEQ ID No: 259), TEC L89R (SEQ ID No: 240), TEC W531R (SEQ ID No: 241), TEC P587L (SEQ ID No: 242), CSK Q26X (SEQ ID No: 52) and ACK1 S985N (SEQ ID No: 14).

2. A method of identifying a cell that is resistant to apoptosis inducing reagents (chemoresistant), the method comprising:
measuring in the cell the expression of the protein kinase Tyro3 and comparing the result of the measurement obtained with that of a control measurement, wherein an increased expression of protein kinase Tyro3 indicates resistance of the cell to apoptosis inducing reagents;
identifying the amino acid at position 531 or 822 of the expressed protein kinase Tyro3, wherein the presence of Leucine at position 531 instead of Serine or the presence of Leucine at position 822 instead of Proline indicates increased resistance of the cell to apoptosis inducing reagents; or
identifying the amino acid at position 89, 531 or 587 of the expressed protein kinase TEC, wherein the presence of Arginine at position 89 instead of Leucine, the presence of Arginine at position 531 instead of Tryptophan, or the presence of Leucine at position 587 instead of Proline indicates increased resistance of the cell to apoptosis inducing reagents.

3. The method of claim 2, wherein the amino acid at position 531 of the expressed protein kinase TEC is identified and wherein the cell is a T cell.

4. The method of claim 2, wherein the amino acid at position 89 of the expressed protein kinase TEC is identified and wherein the cell is a stomach cell.

5. The method of claim 2, wherein the amino acid at position 587 of the expressed protein kinase TEC is identified and wherein the cell is a lung cell.

6. (canceled)

7. A method of identifying a cell having a predisposition to transform into a cancer cell, the method comprising:
identifying the amino acid at position 367 of the expressed protein kinase FGFR4 or the amino acid at position 252 of the expressed protein kinase FGFR1, wherein the presence of Cysteine at position 367 of the expressed protein kinase FGFR4 instead of Tyrosine and/or the presence of Serine at position 252 of the expressed protein kinase FGFR1 instead of Proline indicates an increased predisposition to transform into a cancer cell;
identifying in the cell, the cell being a liver cell, the amino acid at position 388 of the expressed protein kinase FGFR4, wherein the presence of Arginine at position 388 instead of Glycine indicates an increased predisposition to transform into a hepatocellular carcinoma cell;
identifying the amino acid at position 26 of the expressed protein kinase C-terminal Src kinase (CSK), wherein the presence of an amino acid different from Glutamine at position 26 of the expressed protein kinase CSK indicates an increased predisposition to transform into a cancer cell; or
identifying the amino acid at position 985 of the expressed protein kinase Ack1, wherein the presence of Asparagine at position 985 of the expressed protein kinase Ack1 instead of Serine indicates an increased predisposition to transform into a cancer cell.

8. (canceled)

9. The method of claim 7, wherein the amino acid at position 26 of the expressed protein kinase CSK is identified and wherein the cell is a colon cell.

10. The method of claim 7, wherein the presence of Asparagine at position 985 of protein kinase Ack1 renders the protein kinase less susceptible to ubiquitination, thereby rendering the protein kinase more durable than protein kinase Ack1 comprising Serine at position 985, and wherein the cell is a kidney cell.

11. The method of claim 7, wherein the amino acid at position 388 of the expressed protein kinase FGFR4 is identified, and wherein further the genotype of the gene encoding the FGFR4 receptor in the liver cell is determined, wherein the homozygous genotype FGFR4 388Arg indicates an increased predisposition to transform into a hepatocellular carcinoma cell.

12-17. (canceled)

18. The nucleic acid molecule according to claim 1, wherein the nucleic acid molecule is isolated from a natural source, wherein the natural source is a mammal, and wherein the mammal is a human.

19-20. (canceled)

21. The nucleic acid molecule according to claim 1, wherein the nucleic acid molecule is of recombinant origin or wherein the nucleic acid molecule is RNA or DNA.

22-23. (canceled)

24. A nucleic acid probe for the detection of a nucleic acid molecule encoding a mutant kinase polypeptide in a sample, wherein the mutant kinase polypeptide is selected from the group consisting of ACK1, CSK, FGFR1, FGFR4, TEC, and TYRO3, and wherein said mutant kinase polypeptide encoded by said nucleic acid molecule comprises at least one of the mutations ACK1 H37Y (SEQ ID No: 274), ACK1 E111K (SEQ ID No: 275), ACK1 R127H (SEQ ID No: 276), ACK1 M393T (SEQ ID No: 277), ACK1 A634T (SEQ ID No: 278), ACK1 S699N (SEQ ID No: 279), ACK1 P731L (SEQ ID No: 280), ACK1 R748W (SEQ ID No: 281), ACK1 G947D (SEQ ID No: 282), ACK1 S985N (SEQ ID No: 283), CSK Q26X (SEQ ID No: 321), FGFR1 R78H (SEQ ID No: 397), FGFR1 P252S (SEQ ID No: 398), FGFR1 A268S (SEQ ID No: 399), FGFR1 G539_K540del (SEQ ID No: 400), FGFR4 Y367C (SEQ ID No: 402), TEC L89R (SEQ ID No: 509), TEC W531R (SEQ ID No: 510), TEC P587L (SEQ ID No: 511), TYRO3 S324C (SEQ ID No: 524), TYRO3 E489K (SEQ ID No: 525), TYRO3 S531L (SEQ ID No: 526), TYRO3 N788T (SEQ ID No: 527) and TYRO3 P822L (SEQ ID No: 528), wherein said nucleic acid probe contains a nucleotide base sequence that will hybridize to the mutated region of said nucleic acid sequence.

25-27. (canceled)

28. A method for detecting the presence or amount of nucleic acid molecule encoding a mutant kinase polypeptide or a kinase polypeptide variant in a sample comprising the steps of a) contacting the sample with a nucleic acid probe according to claim 24 under conditions such that hybridization occurs and b) detecting the presence or amount of the probe bound to the nucleic acid molecules encoding a mutant kinase polypeptide.

29-32. (canceled)

33. A kit for performing the method of claim 28, including a container means having disposed therein one or more nucleic acid probes according to claim 24.

34-35. (canceled)

36. A recombinant cell or tissue comprising a nucleic acid molecule according to claim 1.

37. (canceled)

38. An isolated, enriched, or purified mutant kinase polypeptide selected from the group consisting of ACK1, CSK, FGFR1, FGFR4, TEC, and TYRO3, wherein said mutant kinase polypeptide comprises at least one of the mutations ACK1 H37Y (SEQ ID No: 5), ACK1 E111K (SEQ ID No: 6), ACK1 R127H (SEQ ID No: 7), ACK1 M393T (SEQ ID No: 8), ACK1 A634T (SEQ ID No: 9), ACK1 S699N (SEQ ID No: 10), ACK1 P731L (SEQ ID No: 11), ACK1 R748W (SEQ ID No: 12), ACK1 G947D (SEQ ID No: 13), ACK1 S985N (SEQ ID No: 14), CSK Q26X (SEQ ID No: 52), FGFR1 R78H (SEQ ID No: 128), FGFR1 P252S (SEQ ID No: 129), FGFR1 A268S (SEQ ID No: 130), FGFR1 G539_K540del (SEQ ID No: 131), FGFR4 Y367C (SEQ ID No: 133), TEC L89R (SEQ ID No: 240), TEC W531R (SEQ ID No: 241), TEC P587L (SEQ ID No: 242), TYRO3 S324C (SEQ ID No: 255), TYRO3 E489K (SEQ ID No: 256), TYRO3 S531L (SEQ ID No: 257), TYRO3 N788T (SEQ ID No: 258) and TYRO3 P822L (SEQ ID No: 259), or a fragment thereof.

39-50. (canceled)

51. A method for detecting the presence or amount of at least one mutant kinase polypeptide or kinase polypeptide variant in a sample, comprising the steps of a) probing the sample with a monoclonal or polyclonal antibody or antibody fragment having specific binding affinity only for a mutant kinase polypeptide according to claim 38 or a mutant kinase polypeptide domain or fragment thereof, under conditions suitable for kinase-antibody immunocomplex formation and b) detecting the presence or amount of the antibody bound to the kinase polypeptide.

52. A kit for performing the method of claim 51, including the antibody or the antibody fragment.

53-54. (canceled)

55. A method for identifying a compound that modulates kinase activity in vitro comprising the steps of: (a) contacting a kinase polypeptide according to claim 38; or a kinase polypeptide selected from the group consisting of ACK1, FGFR4, and TYRO3, wherein said kinase polypeptide comprises at least one of the germline alterations ACK1 R1038H (SEQ ID No. 546), FGFR4 V10I (SEQ ID No. 580), and TYRO3 I346N (SEQ ID No. 655), or any functional fragment thereof, with the proviso that said fragment includes the altered region, or the mutant kinase polypeptide FGFR1 V427_T428del consisting of the amino acid sequence set forth in SEQ ID NO: 577 or the mutant kinase polypeptide FGFR4 G388R consisting of the amino acid sequence set forth in SEQ ID NO: 582 with a test substance; (b) measuring the activity of said polypeptide; and (c) determining whether said substance modulates the activity of said polypeptide.

56-58. (canceled)

59. A method for identifying a compound that modulates kinase activity in vivo comprising the steps of: (a) expressing a kinase polypeptide according to claim 38; or a kinase polypeptide selected from the group consisting of ACK1, FGFR4, TYRO3, wherein said kinase polypeptide comprises at least one of the germline alterations ACK1 R1038H (SEQ ID No: 546), FGFR4 V10I (SEQ ID No: 580) and TYRO3 I346N (SEQ ID No: 655), or any functional fragment thereof, with the proviso that said fragment includes the altered region, or the mutant kinase polypeptide FGFR1 V427_T428del consisting of the amino acid sequence set forth in SEQ ID NO: 577 or the mutant kinase polypeptide FGFR4 G388R consisting of the amino acid sequence set forth in SEQ ID NO:582 in a cell CSK, FGFR1, FGFR4, (b) adding a test substance to said cell; and (c) monitoring a change in cell phenotype or the interaction between said polypeptide and a natural binding partner.

60-62. (canceled)

63. A method for treating or preventing a proliferative disease or disorder by administering to a subject in need of such treatment a substance that modulates the activity of a kinase according to claim 38; or a kinase selected from the group consisting of ACK1, FGFR4, and TYRO3, wherein said kinase polypeptide comprises at least one of the germline alterations ACK1 P725L (SEQ ID No. 545), ACK1 R1038H (SEQ ID No. 546), FGFR4 V10I (SEQ ID No. 580) and TYRO3 I346N (SEQ ID No. 655), or the mutant kinase polypeptide FGFR4 G388R consisting of the amino acid sequence set forth in SEQ ID NO: 582.

64-66. (canceled)

67. A method for the diagnosis of a proliferative disease or disorder or the risk prediction of developing a proliferative disease or disorder in a subject, said disease or disorder being characterized by an abnormality in a signal transduction pathway due to aberrant protein kinase function, wherein said method comprises: (a) providing a biological sample from said subject; (b) contacting the sample with a nucleic acid probe which hybridizes under hybridization assay conditions to a target region of a nucleic acid molecule encoding a mutant kinase polypeptide according to claim 38; or a kinase polypeptide variant selected from the group consisting of ACK1, FGFR4, and TYRO3, wherein the kinase polypeptide variant encoded by said nucleic acid molecule comprises at least one of the germline alterations ACK1 P725L (SEQ ID No. 545), ACK1 R1038H (SEQ ID No. 546), FGFR4 V10I (SEQ ID No. 580), FGFR4 G388R (SEQ ID No. 582), and TYRO3 I346N (SEQ ID No. 655); and (c) detecting the presence or amount of the probe:target region hybrid as an indication of or predisposition to the disease or disorder.

68-74. (canceled)
